(19) United States
(12) Patent Application Publication
(10) Pub. No.: US 2025/0242056 A1
(43) Pub. Date: Jul. 31, 2025

(54) METHODS AND COMPOSITIONS FOR USING PLASMA CELL DEPLETING AGENTS AND/OR B CELL DEPLETING AGENTS TO SUPPRESS HOST ANTI-AAV ANTIBODY RESPONSE AND ENABLE AAV TRANSDUCTION AND RE-DOSING

(71) Applicant: Regeneron Pharmaceuticals, Inc., Tarrytown, NY (US)

(72) Inventors: Nicholas Giovannone, Elmsford, NY (US); Andre Limnander, New York, NY (US); Andrew Baik, Scarsdale, NY (US); Katherine Cygnar, New York, NY (US); Christos Kyratsous, Irvington, NY (US)

(21) Appl. No.: 19/036,194

(22) Filed: Jan. 24, 2025

Related U.S. Application Data
(60) Provisional application No. 63/639,285, filed on Apr. 26, 2024.
(60) Provisional application No. 63/625,654, filed on Jan. 26, 2024.

(51) Int. Cl. A61K 48/00; A61K 9/1271; A61K 9/51; A61K 38/37; A61K 38/47; A61K 38/48; A61K 39/00; C07K 16/28; C12N 9/22; C12N 15/11; C12N 15/86; C12N 15/88

(52) U.S. Cl. CPC A61K 48/005; A61K 9/1271; A61K 9/5123; A61K 38/37; A61K 38/47; A61K 38/4846; A61K 48/0083; C07K 16/2809; C07K 16/2878; C07K 16/2881; C07K 16/2887; C07K 16/2896; C12N 9/22; C12N 15/111; C12N 15/86; C12N 15/88; C12Y 302/0102; C12Y 304/21022; A61K 2039/505; C07K 2317/31; C07K 2317/52; C07K 2317/565; C12N 2310/20; C12N 2750/14143; C12N 2830/50

(57) ABSTRACT

Provided herein are methods of inserting a nucleic acid encoding a polypeptide of interest into a target genomic locus in a cell or a population of cells in a subject, methods of expressing a polypeptide of interest from a target genomic locus in a cell or a population of cells in a subject, methods of treating an enzyme deficiency in a subject in need thereof, and methods of preventing or reducing the onset of a sign or symptom of an enzyme deficiency in a subject in need thereof. Some methods, such as when a subject has preexisting against an immunogen to be administered, use plasma cell depleting agents or combinations comprising plasma cell depleting agents to mitigate immune response and facilitate redosing of nucleic acid constructs encoding a polypeptide of interest and nuclease agents targeting a target genomic locus to achieve, for example, a step-wise increase in expression of a polypeptide of interest in a subject following insertion of the nucleic acid construct without overshooting. Other methods, such as when a subject has no preexisting immunity against an immunogen to be administered, use B cell depleting agents (e.g., anti-CD20×CD3 antibody or functional fragment thereof) to mitigate immune response and facilitate redosing of nucleic acid constructs encoding a polypeptide of interest and nuclease agents targeting a target genomic locus to achieve, for example, a step-wise increase in expression of a polypeptide of interest in a subject following insertion of the nucleic acid construct without overshooting.

### CROSS-REFERENCE TO RELATED APPLICATIONS

This application claims the benefit of U.S. Application No. 63/625,654, filed Jan. 26, 2024, and U.S. Application No. 63/639,285, filed Apr. 26, 2024, each of which is herein incorporated by reference in its entirety for all purposes.

### REFERENCE TO A SEQUENCE LISTING SUBMITTED AS AN XML FILE

The Sequence Listing written in file 624866SEQLIST.xml is 1,816,797 bytes, was created on Jan. 21, 2025, and is hereby incorporated by reference in its entirety.

### BACKGROUND

Adeno-associated virus (AAV)-based vectors hold tremendous promise to transform treatment of genetic diseases. Yet, the potential of AAV gene therapy has so far been limited by development of host antibodies (e.g., neutralizing antibodies (nAbs)) that block transduction or affect uptake on subsequent exposures. Clinically, the inability to re-dose AAVs presents challenges because efficacy cannot be restored if transgene expression is subtherapeutic or lost (e.g., due to cell division, silencing, or a cytotoxic immune response). Moreover, due to natural AAV exposure, many patients develop nAbs prior to treatment that render them ineligible for even a single dose. Therefore, strategies that prevent or attenuate anti-AAV nAb responses could vastly expand the utility and accessibility of existing AAV gene therapies, while safeguarding eligibility for future AAV-based advances.

Similarly, in AAV gene therapies, seronegative/naive patients can be dosed with AAV and develop antibody responses to the AAV capsid antigen. This antibody response prevents future re-dosing of AAV because the antibodies are neutralizing, and the antibody response is sustained for 10+ years.

### SUMMARY

Provided herein are methods of inserting a nucleic acid encoding a polypeptide of interest into a target genomic locus in a cell or a population of cells in a subject, methods of expressing a polypeptide of interest from a target genomic locus in a cell or a population of cells in a subject, methods of treating an enzyme deficiency in a subject in need thereof, and methods of preventing or reducing the onset of a sign or symptom of an enzyme deficiency in a subject in need thereof. Also provided are compositions, combination, or kits, e.g., for use in such methods.

In one aspect, provided are methods of inserting a nucleic acid encoding a polypeptide of interest into a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of a plasma cell depleting agent, wherein the nuclease agent cleaves the nuclease target site, and the nucleic acid construct is inserted into the target genomic locus. In some such methods, provided are methods of inserting a nucleic acid encoding a polypeptide of interest into a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of a plasma cell depleting agent, wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, and the nucleic acid construct is inserted into the target genomic locus.

In another aspect, provided are methods of expressing a polypeptide of interest from a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of a plasma cell depleting agent, wherein the nuclease agent cleaves the nuclease target site, the nucleic acid construct is inserted into the target genomic locus to create a modified target genomic locus, and the polypeptide of interest is expressed from the modified target genomic locus. In some such methods, provided are methods of expressing a polypeptide of interest from a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of a plasma cell depleting agent, wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, the nucleic acid construct is inserted into the target genomic locus to create a modified target genomic locus, and the polypeptide of interest is expressed from the modified target genomic locus.

In another aspect, provided are methods of treating an enzyme deficiency in a subject in need thereof, comprising administering to the subject: (a) a nucleic acid construct comprising a coding sequence for a polypeptide of interest, wherein the polypeptide of interest comprises an enzyme to treat the enzyme deficiency; (b) a nuclease agent or one or more nucleic acids encoding the nuclease agent, wherein the nuclease agent targets a nuclease target site in a target genomic locus; and (c) an effective amount of a plasma cell depleting agent, wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency. In some such methods, provided are methods of treating an enzyme deficiency in a subject in need thereof, comprising administering to the subject: (a) a nucleic acid construct comprising a coding sequence for a polypeptide of interest, wherein the polypeptide of interest comprises an enzyme to treat the enzyme deficiency; (b) a nuclease agent or one or more nucleic acids encoding the nuclease agent, wherein the nuclease agent targets a nuclease target site in a target genomic locus; and (c) an effective amount of a plasma cell depleting agent, wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency.

In another aspect, provided are methods of preventing or reducing the onset of a sign or symptom of an enzyme deficiency in a subject in need thereof, comprising administering to the subject: (a) a nucleic acid construct comprising a coding sequence for a polypeptide of interest, wherein the enzyme deficiency is characterized by a loss-of-function of the polypeptide of interest; (b) a nuclease agent or one or more nucleic acids encoding the nuclease agent, wherein the nuclease agent targets a nuclease target site in a target genomic locus; and (c) an effective amount of a plasma cell depleting agent, wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency. In some such methods, provided are methods of preventing or reducing the onset of a sign or symptom of an enzyme deficiency in a subject in need thereof, comprising administering to the subject: (a) a nucleic acid construct comprising a coding sequence for a polypeptide of interest, wherein the enzyme deficiency is characterized by a loss-of-function of the polypeptide of interest; (b) a nuclease agent or one or more nucleic acids encoding the nuclease agent, wherein the nuclease agent targets a nuclease target site in a target genomic locus; and (c) an effective amount of a plasma cell depleting agent, wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency.

In some such methods, the subject has a disease of a bleeding disorder characterized by the enzyme deficiency, a disease of an inborn error of metabolism characterized by the enzyme deficiency, or a lysosomal storage disease characterized by the enzyme deficiency. Optionally, the disease is hemophilia B and the polypeptide of interest is a factor IX protein, the disease is hemophilia A and the polypeptide of interest is a factor VIII protein, or the disease is Pompe disease and the polypeptide of interest is a multidomain therapeutic protein comprising a delivery domain fused to a lysosomal alpha-glucosidase.

Some such methods further comprise a subsequent administration step comprising administering to the subject at one or more subsequent times: (a) the nucleic acid construct; (b) the nuclease agent or the one or more nucleic acids encoding the nuclease agent; and optionally (c) the plasma cell depleting agent, until a desired level of expression and/or activity of the polypeptide of interest is achieved in the subject. Some such methods further comprise a subsequent administration step comprising administering to the subject at one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the plasma cell depleting agent, until a desired level of expression and/or activity of the polypeptide of interest is achieved in the subject. Some such methods further comprise a subsequent administration step comprising administering to the subject at one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus; and optionally (c) the plasma cell depleting agent, until a desired level of expression and/or activity of the polypeptide of interest is achieved in the subject. Some such methods further comprise a subsequent administration step comprising administering to the subject at one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus; and optionally (c) the plasma cell depleting agent, until a desired level of expression and/or activity of the polypeptide of interest is achieved in the subject. Some such methods comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest is achieved in the subject.

In some such methods, the polypeptide of interest is a factor IX protein, and the desired expression level of the factor IX protein in the subject is a serum level of at least about 3 μg/mL or about 3-5 μg/mL. In some such methods, the polypeptide of interest is a multidomain therapeutic protein comprising a delivery domain fused to a lysosomal alpha-glucosidase, and the desired expression level of the multidomain therapeutic protein in the subject is a serum level of at least about 2 μg/mL or at least about 5 μg/mL.

Some such methods further comprise a subsequent administration step comprising administering to the subject at one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus; and optionally (c) the plasma cell depleting agent, wherein the second nuclease agent cleaves the second nuclease target site, and the second nucleic acid construct is inserted into the second target genomic locus.

In some such methods, the one or more subsequent administration steps is one subsequent administration step. In some such methods, the one or more subsequent administration steps is two subsequent administration steps or comprises at least two subsequent administration steps. In some such methods, the plasma cell depleting agent is administered in the one or more subsequent administration steps if there is no preexisting plasma cell depleting agent in the subject or if preexisting plasma cell depleting agent expression and/or activity levels are below a desired threshold level. Optionally, the method comprises measuring the plasma cell depleting agent expression and/or activity levels prior to the one or more subsequent administration steps. In some such methods, the plasma cell depleting agent is capable of depleting long-lived plasma cells (LLPC).

In some such methods, the plasma cell depleting agent is a B cell maturation antigen (BCMA) targeting agent. In some such methods, the BCMA targeting agent is a chimeric antigen receptor against BCMA or an anti-BCMA antibody or a functional fragment thereof. In some such methods, the anti-BCMA antibody or functional fragment thereof is conjugated to a cytotoxic agent. In some such methods, the anti-BCMA antibody is a multispecific antibody or a functional fragment thereof. In some such methods, the multispecific anti-BCMA antibody or functional fragment thereof targets BCMA and CD3. In some such methods, the multispecific anti-BCMA antibody or functional fragment thereof is anti-BCMA×CD3 bispecific antibody or functional fragment thereof. In some such methods, the multispecific anti-BCMA antibody or functional fragment thereof is anti-BCMA×CD3 bispecific antibody or functional fragment thereof. In some such methods, the anti-BCMA×CD3 bispecific antibody is selected from linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B.

In some such methods, the anti-BCMA×CD3 bispecific antibody or functional fragment thereof comprises a first antigen-binding domain that specifically binds to BCMA comprising three heavy chain CDRs (HCDR1, HCDR2 and HCDR3) contained within a heavy chain variable region (HCVR) comprising the amino acid sequence of SEQ ID NO: 2, and three light chain CDRs (LCDR1, LCDR2 and LCDR3) contained within a light chain variable region (LCVR) comprising the amino acid sequence of SEQ ID NO: 18. In some such methods, the first antigen-binding domain that specifically binds to BCMA comprises a HCDR1 comprising the amino acid sequence of SEQ ID NO: 4, a HCDR2 comprising the amino acid sequence of SEQ ID NO: 6, a HCDR3 comprising the amino acid sequence of SEQ ID NO: 8, a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24.

In some such methods, the anti-BCMA×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence selected from the group consisting of SEQ ID NOS: 26 and 34, and three light chain CDRs (LCDR1, LCDR2 and LCDR3) contained within a light chain variable region (LCVR) comprising the amino acid sequence of SEQ ID NO: 18. In some such methods, the second antigen-binding domain that specifically binds to CD3 comprises a HCDR1 comprising the amino acid sequence of SEQ ID NO: 28 or 36, a HCDR2 comprising the amino acid sequence of SEQ ID NO: 30 or 38, a HCDR3 comprising the amino acid sequence of SEQ ID NO: 32 or 40, a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24.

In some such methods, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 4, 6, and 8, respectively, and LCDR1, LCDR2, and LCDR3 comprising the amino acid sequences of SEQ ID NOS: 20, 22, and 24, respectively; and (b) a second antigen-binding domain that comprises HCDR1, HCDR2, and HCDR3 comprising the amino acid sequences of SEQ ID NOS: 28, 30, and 32, respectively, and LCDR1, LCDR2, and LCDR3 comprising the amino acid sequences of SEQ ID NOS: 20, 22, and 24, respectively. In some such methods, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 4, 6, and 8, respectively, and LCDR1, LCDR2, and LCDR3 comprising the amino acid sequences of SEQ ID NOS: 20, 22, and 24, respectively; and (b) a second antigen-binding domain that comprises HCDR1, HCDR2, and HCDR3 comprising the amino acid sequences of SEQ ID NOS: 36, 38, and 40, respectively, and LCDR1, LCDR2, and LCDR3 comprising the amino acid sequences of SEQ ID NOS: 20, 22, and 24, respectively. In some such methods, the anti-BCMA×CD3 bispecific antibody or functional fragment thereof comprises a human IgG heavy chain constant region, optionally wherein the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such methods, the human IgG heavy chain constant region is isotype IgG4 or IgG1.

Some such methods further comprise administering to the subject an effective amount of a B cell depleting agent and/or an immunoglobulin depleting agent. Some such methods further comprise administering to the subject an effective amount of a B cell depleting agent and an immunoglobulin depleting agent. In some such methods, the B cell depleting agent is administered before, at the same time as, or after the plasma cell depleting agent. In some such methods, the B cell depleting agent is administered prior to and after the nucleic acid construct. In some such methods, the immunoglobulin depleting agent is administered prior to and after the nucleic acid construct. In some such methods, the immunoglobulin depleting agent is administered after the plasma cell depleting agent. In some embodiments, the immunoglobulin depleting agent is administered after an initial dose of the plasma cell depleting agent, or wherein the immunoglobulin depleting agent is administered after an initial dose of the plasma cell depleting agent and after an initial dose of the B cell depleting agent. In some such methods, the B cell depleting agent is capable of depleting B cells and plasma cells that express low levels of BCMA. In some such methods, the B cell depleting agent is an agent that binds to a B cell surface molecule. In some such methods, the B cell depleting agent is selected from an anti-CD19 antibody, an anti-CD20 antibody, an anti-CD22 antibody, an anti-CD79 antibody, an anti-CD20×CD3 bispecific antibody, an anti-CD19×CD3 bispecific antibody, an anti-CD22×CD3 bispecific antibody, an anti-CD79×CD3 bispecific antibody, functional fragments of any of said antibodies, and any combinations thereof. In some such methods, the B cell depleting agent is selected from an anti-CD19 antibody, an anti-CD20 antibody, an anti-CD19 antibody and an anti-CD20 antibody, an anti-CD22 antibody, an anti-CD79 antibody, an anti-CD20×CD3 bispecific antibody, an anti-CD19×CD3 bispecific antibody, an anti-CD22×CD3 bispecific antibody, an anti-CD79×CD3 bispecific antibody, functional fragments of any of said antibodies, and any combinations thereof. In some such methods, the B cell depleting agent comprises an anti-CD20 antibody or a functional fragment thereof and an anti-CD19 antibody or a functional fragment thereof.

In some such methods, the B cell depleting agent is an anti-CD20 antibody or a functional fragment thereof, wherein the anti-CD20 antibody is a multispecific antibody or a functional fragment thereof. In some such methods, the multispecific anti-CD20 antibody or functional fragment thereof targets CD20 and CD3. In some such methods, the multispecific anti-CD20 antibody or functional fragment thereof is anti-CD20×CD3 bispecific antibody or functional fragment thereof.

In some such methods, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45. In some such methods, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such methods, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45. In some such methods, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such methods, the anti-CD20×CD3 bispecific antibody or functional fragment thereof comprises: (a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and (b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively. In some such methods, the anti-CD20×CD3 bispecific antibody or functional fragment thereof comprises a human IgG heavy chain constant region, optionally wherein the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such methods, the human IgG heavy chain constant region is isotype IgG4 or IgG1.

In some such methods, the B cell depleting agent is an agent targeting a B cell survival factor. In some such methods, the B cell depleting agent is a BLyS/BAFF inhibitor, an APRIL inhibitor, a BLyS receptor 3/BAFF receptor inhibitor, or any combination thereof. In some such methods, the immunoglobulin depleting agent is capable of accelerating IgG clearance. In some such methods, the immunoglobulin depleting agent is a neonatal Fc receptor (FcRn) blocker. In some such methods, the FcRn blocker is selected from Efgartigimod (ARGX-113), Rozanolixizumab (UCB7665), Batoclimab (RVT-1401), IMVT-1402, Nipocalimab (M281), Orilanolimab (SYNT001), and any combinations thereof.

Some such methods, further comprise plasmapheresis, therapeutic plasma exchange, or immunoadsorption. In some such methods, the plasma cell depleting agent is administered simultaneously with the nucleic acid construct. In some such methods, the plasma cell depleting agent is administered prior to the nucleic acid construct. In some such methods, the plasma cell depleting agent is administered prior to and after the nucleic acid construct. In some such methods, the plasma cell depleting agent is administered within about 6 months after the nucleic acid construct. Optionally, the nucleic acid construct is in a viral vector, and the plasma cell depleting agent is administered if the viral vector is still present in the subject. In some such methods, the nucleic acid construct is administered within about 3 months, within about 2 months, within about 7 weeks, within about 6 weeks, within about 5 weeks, within about 4 weeks, within about 3 weeks, or within about 2 weeks after an initial dose of the plasma cell depleting agent, or the nucleic acid construct is administered at least about 2 weeks, at least about 3 weeks, at least about 4 weeks, at least about 5 weeks, at least about 6 weeks, at least about 7 weeks, at least about 2 months, or at least about 3 months after an initial dose of the plasma cell depleting agent. In some such methods, the nucleic acid construct is administered about 2 weeks to about 7 weeks, about 3 weeks to about 6 weeks, or about 4 weeks to about 5 weeks after an initial dose of the plasma cell depleting agent. In some such methods, the plasma cell depleting agent is administered about 1 week prior to or within about 1 week prior to the nucleic acid construct. In some such methods, the nucleic acid construct is administered simultaneously with the nuclease agent or the one or more nucleic acids encoding the nuclease agent. In some such methods, the nucleic acid construct is administered prior to or after the nuclease agent or the one or more nucleic acids encoding the nuclease agent.

In some such methods, the nucleic acid construct is in the nucleic acid vector. Optionally, the nucleic acid vector is a viral vector. Optionally, the viral vector is administered at a dose of about 3E11 vg/kg to about 5E13 vg/kg. In some such methods, the nucleic acid vector is an adeno-associated viral (AAV) vector. Optionally, the nucleic acid construct is flanked by inverted terminal repeats (ITRs) on each end. Optionally, the ITR on at least one end comprises, consists essentially of, or consists of SEQ ID NO: 283. Optionally, the ITR on each end comprises, consists essentially of, or consists of SEQ ID NO: 283. Optionally, the ITR on at least one end comprises, consists essentially of, or consists of SEQ ID NO: 281. Optionally, the ITR on each end comprises, consists essentially of, or consists of SEQ ID NO: 281. In some such methods, the AAV vector is a single-stranded AAV (ssAAV) vector. In some such methods, the AAV vector is a recombinant AAV8 (rAAV8) vector.

In some such methods, the polypeptide of interest is a factor IX protein. In some such methods, the factor IX protein coding sequence encodes a factor IX protein comprising SEQ ID NO: 97. In some such methods, the factor IX protein coding sequence comprises or consists of SEQ ID NO: 68, or wherein the factor IX protein coding sequence comprises or consists of SEQ ID NO: 61.

In some such methods, the nucleic acid construct is a bidirectional construct, wherein the factor IX protein coding sequence is a first factor IX protein coding sequence, and the bidirectional construct further comprises a reverse complement of a second factor IX protein coding sequence, wherein the first factor IX protein coding sequence and the second factor IX protein coding sequence are different but encode the same factor IX protein sequence. In some such methods, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 61 and the second factor IX protein coding sequence comprises SEQ ID NO: 68; or (ii) the first factor IX protein coding sequence comprises SEQ ID NO: 68 and the second factor IX protein coding sequence comprises SEQ ID NO: 61, wherein the nucleic acid construct does not comprise a promoter that drives the expression of the factor IX protein, and wherein the nucleic acid construct does not comprise homology arms. In some such methods, the nucleic acid construct comprises SEQ ID NO: 109 or 82 or the reverse complement thereof.

In some such methods, the nucleic acid construct is a unidirectional construct. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 61 or SEQ ID NO: 68, wherein the nucleic acid construct does not comprise a promoter that drives the expression of the factor IX protein, and wherein the nucleic acid construct does not comprise homology arms.

In some such methods, the polypeptide of interest is a multidomain therapeutic protein comprising a delivery domain fused to a lysosomal alpha-glucosidase. In some such methods, the lysosomal alpha-glucosidase comprises or consists of the sequence set forth in SEQ ID NO: 296. In some such methods, the lysosomal alpha-glucosidase coding sequence comprises or consist of the sequence set forth in SEQ ID NO: 857. In some such methods, the delivery domain is a CD63-binding delivery domain. In some such methods, the CD63-binding delivery domain comprises an anti-CD63 antigen-binding protein. In some such methods, the CD63-binding delivery domain is a single-chain variable fragment (scFv). In some such methods, the scFv comprises or consists of the sequence set forth in SEQ ID NO: 306. In some such methods, the scFv coding sequence comprises or consists of the sequence set forth in SEQ ID NO: 866. In some such methods, the multidomain therapeutic protein comprises or consists of the sequence set forth in SEQ ID NO: 316. In some such methods, the coding sequence for the multidomain therapeutic protein comprises or consists of the sequence set forth in SEQ ID NO: 863. Optionally, the nucleic acid construct comprises the sequence set forth in SEQ ID NO: 900 or 884.

In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 863, optionally wherein the nucleic acid construct comprises the sequence set forth in SEQ ID NO: 900 or 884, wherein the polyadenylation signal comprises a BGH polyadenylation signal and a unidirectional SV40 late polyadenylation signal, optionally wherein the BGH polyadenylation signal comprises the sequence set forth in SEQ ID NO: 858 and the unidirectional SV40 late polyadenylation signal comprises the sequence set forth in SEQ ID NO: 859, optionally wherein the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such methods, the delivery domain is a TfR-binding delivery domain. In some such methods, the TfR-binding delivery domain comprises an anti-TfR antigen-binding protein. In some such methods, the anti-TfR antigen-binding protein comprises a HCVR comprising the HCDR1, HCDR2 and HCDR3 of a HCVR that comprises the amino acid sequence set forth in SEQ ID NO: 555 (or a variant thereof); and a LCVR comprising the LCDR1, LCDR2 and LCDR3 of a LCVR that comprises the amino acid sequence set forth in SEQ ID NO: 560 (or a variant thereof). In some such methods, the anti-TfR antigen-binding protein comprises a HCVR that comprises: an HCDR1 comprising the amino acid sequence set forth in SEQ ID NO: 556 (or a variant thereof), an HCDR2 comprising the amino acid sequence set forth in SEQ ID NO: 557 (or a variant thereof), and an HCDR3 comprising the amino acid sequence set forth in SEQ ID NO: 558 (or a variant thereof); and a LCVR that comprises: an LCDR1 comprising the amino acid sequence set forth in SEQ ID NO: 561 (or a variant thereof), an LCDR2 comprising the amino acid sequence set forth in SEQ ID NO: 562 (or a variant thereof), and an LCDR3 comprising the amino acid sequence set forth in SEQ ID NO: 563 (or a variant thereof). In some such methods, the anti-TfR antigen-binding protein comprises a HCVR that comprises the amino acid sequence set forth in SEQ ID NO: 555 (or a variant thereof); and a LCVR that comprises the amino acid sequence set forth in SEQ ID NO: 560 (or a variant thereof).

In some such methods, the TfR-binding delivery domain comprises a single-chain variable fragment (scFv). In some such methods, the scFv comprises or consists of the sequence set forth in SEQ ID NO: 672. In some such methods, the scFv coding sequence comprises or consists of the sequence set forth in SEQ ID NO: 713. In some such methods, the multidomain therapeutic protein comprises or consists of the sequence set forth in SEQ ID NO: 691. In some such methods, the coding sequence for the multidomain therapeutic protein comprises or consists of the sequence set forth in SEQ ID NO: 852. Optionally, the nucleic acid construct comprises the sequence set forth in SEQ ID NO: 887 or 871.

In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 852, optionally wherein the nucleic acid construct comprises the sequence set forth in SEQ ID NO: 887 or 871, wherein the polyadenylation signal comprises a BGH polyadenylation signal and a unidirectional SV40 late polyadenylation signal, optionally wherein the BGH polyadenylation signal comprises the sequence set forth in SEQ ID NO: 858 and the unidirectional SV40 late polyadenylation signal comprises the sequence set forth in SEQ ID NO: 859, optionally wherein the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such methods, the polypeptide of interest is a factor VIII protein. In some such methods, the polypeptide of interest is an antigen-binding protein, optionally wherein the antigen-binding protein is an antibody. In some such methods, the target genomic locus is an albumin gene, optionally wherein the albumin gene is a human albumin gene. In some such methods, the nuclease target site is in intron 1 of the albumin gene.

In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the DNA-targeting segment comprises any one of SEQ ID NOS: 153-184. Optionally, the DNA-targeting segment comprises any one of SEQ ID NOS: 159, 153, 156, and 164. In some such methods, the DNA-targeting segment consists of any one of SEQ ID NOS: 153-184. Optionally, the DNA-targeting segment consists of any one of SEQ ID NOS: 159, 153, 156, and 164. In some such methods, the guide RNA comprises any one of SEQ ID NOS: 185-248, optionally wherein the guide RNA comprises any one of SEQ ID NOS: 191, 223, 185, 217, 188, 220, 196, and 228. In some such methods, the DNA-targeting segment comprises or consists of SEQ ID NO: 159. In some such methods, the guide RNA comprises SEQ ID NO: 191 or 223. Some such methods comprise administering the guide RNA in the form of RNA.

In some such methods, the guide RNA comprises at least one modification. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. Some such methods comprise administering the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein. Optionally, the Cas protein is derived from a Streptococcus pyogenes Cas9 protein. In some such methods, the Cas protein comprises the sequence set forth in SEQ ID NO: 134. Some such methods comprise administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is fully substituted with N1-methyl-pseudouridine. In some such methods, the mRNA encoding the Cas protein comprises the sequence set forth in SEQ ID NO: 124 or 125. Some such methods comprise administering the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

Some such methods comprise administering the guide RNA in the form of RNA, and the guide RNA comprises SEQ ID NO: 191 or 223, and the method comprises administering the nucleic acid encoding the Cas protein, wherein the nucleic acid comprises an mRNA encoding the Cas protein, and the mRNA encoding the Cas protein comprises the sequence set forth in SEQ ID NO: 124 or 125. Some such methods comprise administering the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and the method comprises administering the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or the neutral lipid is distearoylphosphatidylcholine or 1,2-distearoyl-sn-glycero-3-phosphocholine (DSPC), and/or the helper lipid is cholesterol, and/or the stealth lipid is 1,2-dimyristoyl-rac-glycero-3-methoxypolyethylene glycol-2000. In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol % Lipid A, about 9 mol % DSPC, about 38 mol % cholesterol, and about 3 mol % PEG2k-DMG.

In some such methods, the cell is a liver cell or a hepatocyte, or the population of cells is a population of liver cells or hepatocytes. In some such methods, the subject is a human subject. In some such methods, the subject is a neonatal subject. In some such methods, the subject has preexisting AAV immunity. In some such methods, the nucleic acid vector is in an adeno-associated viral (AAV) vector, and the subject has preexisting AAV immunity. In some such methods, the method further comprises determining whether the subject has immunity against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent prior to the administering. Optionally, the determining comprises determining the presence of neutralizing antibodies against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent.

In another aspect, provided are compositions or combinations comprising an effective amount of a plasma cell depleting agent in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus.

In some such compositions or combinations, the plasma cell depleting agent is capable of depleting long-lived plasma cells (LLPC). In some such compositions or combinations, the plasma cell depleting agent is a B cell maturation antigen (BCMA) targeting agent. In some such compositions or combinations, the BCMA targeting agent is a chimeric antigen receptor against BCMA or an anti-BCMA antibody or a functional fragment thereof. In some such compositions or combinations, the anti-BCMA antibody or functional fragment thereof is conjugated to a cytotoxic agent. In some such compositions or combinations, the anti-BCMA antibody is a multispecific antibody or a functional fragment thereof. In some such compositions or combinations, the multispecific anti-BCMA antibody or functional fragment thereof targets BCMA and CD3. In some such compositions or combinations, the multispecific anti-BCMA antibody or functional fragment thereof is anti-BCMA×CD3 bispecific antibody or functional fragment thereof. In some such compositions or combinations, the anti-BCMA×CD3 bispecific antibody is selected from linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B.

In some such compositions or combinations, the anti-BCMA×CD3 bispecific antibody or functional fragment thereof comprises a first antigen-binding domain that specifically binds to BCMA comprising three heavy chain CDRs (HCDR1, HCDR2 and HCDR3) contained within a heavy chain variable region (HCVR) comprising the amino acid sequence of SEQ ID NO: 2, and three light chain CDRs (LCDR1, LCDR2 and LCDR3) contained within a light chain variable region (LCVR) comprising the amino acid sequence of SEQ ID NO: 18. In some such compositions or combinations, the first antigen-binding domain that specifically binds to BCMA comprises a HCDR1 comprising the amino acid sequence of SEQ ID NO: 4, a HCDR2 comprising the amino acid sequence of SEQ ID NO: 6, a HCDR3 comprising the amino acid sequence of SEQ ID NO: 8, a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24.

In some such compositions or combinations, the anti-BCMA×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence selected from the group consisting of SEQ ID NOS: 26 and 34, and three light chain CDRs (LCDR1, LCDR2 and LCDR3) contained within a light chain variable region (LCVR) comprising the amino acid sequence of SEQ ID NO: 18. In some such compositions or combinations, the second antigen-binding domain that specifically binds to CD3 comprises a HCDR1 comprising the amino acid sequence of SEQ ID NO: 28 or 36, a HCDR2 comprising the amino acid sequence of SEQ ID NO: 30 or 38, a HCDR3 comprising the amino acid sequence of SEQ ID NO: 32 or 40, a LCDR1 comprising the amino acid sequence of SEQ ID NO: 20, a LCDR2 comprising the amino acid sequence of SEQ ID NO: 22, and a LCDR3 comprising the amino acid sequence of SEQ ID NO: 24.

In some such compositions or combinations, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 4, 6, and 8, respectively, and LCDR1, LCDR2, and LCDR3 comprising the amino acid sequences of SEQ ID NOS: 20, 22, and 24, respectively; and (b) a second antigen-binding domain that comprises HCDR1, HCDR2, and HCDR3 comprising the amino acid sequences of SEQ ID NOS: 28, 30, and 32, respectively, and LCDR1, LCDR2, and LCDR3 comprising the amino acid sequences of SEQ ID NOS: 20, 22, and 24, respectively. In some such compositions or combinations, the anti-BCMA×CD3 bispecific antibody or functional fragment thereof comprises: (a) a first antigen-binding domain that comprises HCDR1, HCDR2, and HCDR3 comprising the amino acid sequences of SEQ ID NOS: 4, 6, and 8, respectively, and LCDR1, LCDR2, and LCDR3 comprising the amino acid sequences of SEQ ID NOS: 20, 22, and 24, respectively; and (b) a second antigen-binding domain that comprises HCDR1, HCDR2, and HCDR3 comprising the amino acid sequences of SEQ ID NOS: 36, 38, and 40, respectively, and LCDR1, LCDR2, and LCDR3 comprising the amino acid sequences of SEQ ID NOS: 20, 22, and 24, respectively. In some such compositions or combinations, the anti-BCMA×CD3 bispecific antibody or functional fragment thereof comprises a human IgG heavy chain constant region, optionally wherein the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such compositions or combinations, the human IgG heavy chain constant region is isotype IgG4 or IgG1.

In some such compositions or combinations, the plasma cell depleting agent is further in combination with an effective amount of a B cell depleting agent and/or an immunoglobulin depleting agent. In some such compositions or combinations, the plasma cell depleting agent is further in combination with an effective amount of a B cell depleting agent and an immunoglobulin depleting agent. In some such compositions or combinations, the B cell depleting agent is capable of depleting B cells and plasma cells that express low levels of BCMA. In some such compositions or combinations, the B cell depleting agent is an agent that binds to a B cell surface molecule. In some such compositions or combinations, the B cell depleting agent is selected from an anti-CD19 antibody, an anti-CD20 antibody, an anti-CD22 antibody, an anti-CD79 antibody, an anti-CD20×CD3 bispecific antibody, an anti-CD19×CD3 bispecific antibody, an anti-CD22×CD3 bispecific antibody, an anti-CD79×CD3 bispecific antibody, functional fragments of any of said antibodies, and any combinations thereof. In some such compositions or combinations, the B cell depleting agent is selected from an anti-CD19 antibody, an anti-CD20 antibody, an anti-CD19 antibody and an anti-CD20 antibody, an anti-CD22 antibody, an anti-CD79 antibody, an anti-CD20×CD3 bispecific antibody, an anti-CD19×CD3 bispecific antibody, an anti-CD22×CD3 bispecific antibody, an anti-CD79×CD3 bispecific antibody, functional fragments of any of said antibodies, and any combinations thereof. In some such compositions or combinations, the B cell depleting agent comprises an anti-CD20 antibody or a functional fragment thereof and an anti-CD19 antibody or a functional fragment thereof. In some such compositions or combinations, the B cell depleting agent is an anti-CD20 antibody or a functional fragment thereof, wherein the anti-CD20 antibody is a multispecific antibody or a functional fragment thereof. In some such compositions or combinations, the multispecific anti-CD20 antibody or functional fragment thereof targets CD20 and CD3. In some such compositions or combinations, the multispecific anti-CD20 antibody or functional fragment thereof is anti-CD20×CD3 bispecific antibody or functional fragment thereof.

In some such compositions or combinations, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45. In some such compositions or combinations, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such compositions or combinations, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45. In some such compositions or combinations, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such compositions or combinations, the anti-CD20×CD3 bispecific antibody or functional fragment thereof comprises: (a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and (b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively. In some such compositions or combinations, the anti-CD20×CD3 bispecific antibody or functional fragment thereof comprises a human IgG heavy chain constant region, optionally wherein the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such compositions or combinations, the human IgG heavy chain constant region is isotype IgG4 or IgG1.

In some such compositions or combinations, the B cell depleting agent is an agent targeting a B cell survival factor. In some such compositions or combinations, the B cell depleting agent is a BLyS/BAFF inhibitor, an APRIL inhibitor, a BLyS receptor 3/BAFF receptor inhibitor, or any combination thereof. In some such compositions or combinations, the immunoglobulin depleting agent is capable of accelerating IgG clearance. In some such compositions or combinations, the immunoglobulin depleting agent is a neonatal Fc receptor (FcRn) blocker. In some such compositions or combinations, the FcRn blocker is selected from Efgartigimod (ARGX-113), Rozanolixizumab (UCB7665), Batoclimab (RVT-1401), IMVT-1402, Nipocalimab (M281), Orilanolimab (SYNT001), and any combinations thereof.

In some such compositions or combinations, the nucleic acid construct is in the nucleic acid vector. Optionally, the nucleic acid vector is a viral vector. In some such compositions or combinations, the nucleic acid vector is an adeno-associated viral (AAV) vector. Optionally, the nucleic acid construct is flanked by inverted terminal repeats (ITRs) on each end. Optionally, the ITR on at least one end comprises, consists essentially of, or consists of SEQ ID NO: 283. Optionally, the ITR on each end comprises, consists essentially of, or consists of SEQ ID NO: 283. Optionally, the ITR on at least one end comprises, consists essentially of, or consists of SEQ ID NO: 281. Optionally, the ITR on each end comprises, consists essentially of, or consists of SEQ ID NO: 281. In some such compositions or combinations, the AAV vector is a single-stranded AAV (ssAAV) vector. In some such compositions or combinations, the AAV vector is a recombinant AAV8 (rAAV8) vector.

In some such compositions or combinations, the polypeptide of interest is a factor IX protein. In some such compositions or combinations, the factor IX protein coding sequence encodes a factor IX protein comprising SEQ ID NO: 97. In some such compositions or combinations, the factor IX protein coding sequence comprises or consists of SEQ ID NO: 68, or the factor IX protein coding sequence comprises or consists of SEQ ID NO: 61.

In some such compositions or combinations, the nucleic acid construct is a bidirectional construct, wherein the factor IX protein coding sequence is a first factor IX protein coding sequence, and the bidirectional construct further comprises a reverse complement of a second factor IX protein coding sequence, wherein the first factor IX protein coding sequence and the second factor IX protein coding sequence are different but encode the same factor IX protein sequence. In some such compositions or combinations, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 61 and the second factor IX protein coding sequence comprises SEQ ID NO: 68; or (ii) the first factor IX protein coding sequence comprises SEQ ID NO: 68 and the second factor IX protein coding sequence comprises SEQ ID NO: 61, wherein the nucleic acid construct does not comprise a promoter that drives the expression of the factor IX protein, and wherein the nucleic acid construct does not comprise homology arms. In some such compositions or combinations, the nucleic acid construct comprises SEQ ID NO: 109 or 82 or the reverse complement thereof.

In some such compositions or combinations, the nucleic acid construct is a unidirectional construct. In some such compositions or combinations, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 61 or SEQ ID NO: 68, wherein the nucleic acid construct does not comprise a promoter that drives the expression of the factor IX protein, and wherein the nucleic acid construct does not comprise homology arms.

In some such compositions or combinations, the polypeptide of interest is a multidomain therapeutic protein comprising a delivery domain fused to a lysosomal alpha-glucosidase. In some such compositions or combinations, the lysosomal alpha-glucosidase comprises or consists of the sequence set forth in SEQ ID NO: 296. In some such compositions or combinations, the lysosomal alpha-glucosidase coding sequence comprises or consist of the sequence set forth in SEQ ID NO: 857. In some such compositions or combinations, the delivery domain is a CD63-binding delivery domain. In some such compositions or combinations, the CD63-binding delivery domain comprises an anti-CD63 antigen-binding protein. In some such compositions or combinations, the CD63-binding delivery domain is a single-chain variable fragment (scFv). In some such compositions or combinations, the scFv comprises or consists of the sequence set forth in SEQ ID NO: 306. In some such compositions or combinations, the scFv coding sequence comprises or consists of the sequence set forth in SEQ ID NO: 866. In some such compositions or combinations, the multidomain therapeutic protein comprises or consists of the sequence set forth in SEQ ID NO: 316. In some such compositions or combinations, the coding sequence for the multidomain therapeutic protein comprises or consists of the sequence set forth in SEQ ID NO: 863. Optionally, the nucleic acid construct comprises the sequence set forth in SEQ ID NO: 900 or 884.

In some such compositions or combinations, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 863, optionally wherein the nucleic acid construct comprises the sequence set forth in SEQ ID NO: 900 or 884, wherein the polyadenylation signal comprises a BGH polyadenylation signal and a unidirectional SV40 late polyadenylation signal, optionally wherein the BGH polyadenylation signal comprises the sequence set forth in SEQ ID NO: 858 and the unidirectional SV40 late polyadenylation signal comprises the sequence set forth in SEQ ID NO: 859, optionally wherein the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such compositions or combinations, the delivery domain is a TfR-binding delivery domain. In some such compositions or combinations, the TfR-binding delivery domain comprises an anti-TfR antigen-binding protein. In some such compositions or combinations, the anti-TfR antigen-binding protein comprises a HCVR comprising the HCDR1, HCDR2 and HCDR3 of a HCVR that comprises the amino acid sequence set forth in SEQ ID NO: 555 (or a variant thereof); and a LCVR comprising the LCDR1, LCDR2 and LCDR3 of a LCVR that comprises the amino acid sequence set forth in SEQ ID NO: 560 (or a variant thereof). In some such compositions or combinations, the anti-TfR antigen-binding protein comprises a HCVR that comprises: an HCDR1 comprising the amino acid sequence set forth in SEQ ID NO: 556 (or a variant thereof), an HCDR2 comprising the amino acid sequence set forth in SEQ ID NO: 557 (or a variant thereof), and an HCDR3 comprising the amino acid sequence set forth in SEQ ID NO: 558 (or a variant thereof); and a LCVR that comprises: an LCDR1 comprising the amino acid sequence set forth in SEQ ID NO: 561 (or a variant thereof), an LCDR2 comprising the amino acid sequence set forth in SEQ ID NO: 562 (or a variant thereof), and an LCDR3 comprising the amino acid sequence set forth in SEQ ID NO: 563 (or a variant thereof). In some such compositions or combinations, the anti-TfR antigen-binding protein comprises a HCVR that comprises the amino acid sequence set forth in SEQ ID NO: 555 (or a variant thereof); and a LCVR that comprises the amino acid sequence set forth in SEQ ID NO: 560 (or a variant thereof).

In some such compositions or combinations, the TfR-binding delivery domain comprises a single-chain variable fragment (scFv). In some such compositions or combinations, the scFv comprises or consists of the sequence set forth in SEQ ID NO: 672. In some such compositions or combinations, the scFv coding sequence comprises or consists of the sequence set forth in SEQ ID NO: 713. In some such compositions or combinations, the multidomain therapeutic protein comprises or consists of the sequence set forth in SEQ ID NO: 691. In some such compositions or combinations, the coding sequence for the multidomain therapeutic protein comprises or consists of the sequence set forth in SEQ ID NO: 852. Optionally, the nucleic acid construct comprises the sequence set forth in SEQ ID NO: 887 or 871.

In some such compositions or combinations, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 852, optionally wherein the nucleic acid construct comprises the sequence set forth in SEQ ID NO: 887 or 871, wherein the polyadenylation signal comprises a BGH polyadenylation signal and a unidirectional SV40 late polyadenylation signal, optionally wherein the BGH polyadenylation signal comprises the sequence set forth in SEQ ID NO: 858 and the unidirectional SV40 late polyadenylation signal comprises the sequence set forth in SEQ ID NO: 859, optionally wherein the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such compositions or combinations, the polypeptide of interest is a factor VIII protein. In some such compositions or combinations, the polypeptide of interest is an antigen-binding protein, optionally wherein the antigen-binding protein is an antibody. In some such compositions or combinations, the target genomic locus is an albumin gene, optionally wherein the albumin gene is a human albumin gene. In some such compositions or combinations, the nuclease target site is in intron 1 of the albumin gene.

In some such compositions or combinations, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions or combinations, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions or combinations, the DNA-targeting segment comprises any one of SEQ ID NOS: 153-184. Optionally, the DNA-targeting segment comprises any one of SEQ ID NOS: 159, 153, 156, and 164. In some such compositions or combinations, the DNA-targeting segment consists of any one of SEQ ID NOS: 153-184. Optionally, the DNA-targeting segment consists of any one of SEQ ID NOS: 159, 153, 156, and 164. In some such compositions or combinations, the guide RNA comprises any one of SEQ ID NOS: 185-248. Optionally, the guide RNA comprises any one of SEQ ID NOS: 191, 223, 185, 217, 188, 220, 196, and 228. In some such compositions or combinations, the DNA-targeting segment comprises or consists of SEQ ID NO: 159. In some such compositions or combinations, the guide RNA comprises SEQ ID NO: 191 or 223. In some such compositions or combinations, the composition or combination comprises the guide RNA in the form of RNA.

In some such compositions or combinations, the guide RNA comprises at least one modification. In some such compositions or combinations, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. In some such compositions or combinations, the composition or combination comprises the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions or combinations, the Cas protein is a Cas9 protein. Optionally, the Cas protein is derived from a Streptococcus pyogenes Cas9 protein. In some such compositions or combinations, the Cas protein comprises the sequence set forth in SEQ ID NO: 134. In some such compositions or combinations, the composition or combination comprises the nucleic acid encoding the Cas protein, wherein the nucleic acid comprises an mRNA encoding the Cas protein. In some such compositions or combinations, the mRNA encoding the Cas protein comprises at least one modification. In some such compositions or combinations, the mRNA encoding the Cas protein is fully substituted with N1-methyl-pseudouridine. In some such compositions or combinations, the mRNA encoding the Cas protein comprises the sequence set forth in SEQ ID NO: 124 or 125.

In some such compositions or combinations, the composition or combination comprises the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail. In some such compositions or combinations, the composition or combination comprises the guide RNA in the form of RNA, and the guide RNA comprises SEQ ID NO: 191 or 223, and the composition or combination comprises administering the nucleic acid encoding the Cas protein, wherein the nucleic acid comprises an mRNA encoding the Cas protein, and the mRNA encoding the Cas protein comprises the sequence set forth in SEQ ID NO: 124 or 125.

In some such compositions or combinations, the composition or combination comprises the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, wherein the composition or combination comprises the nucleic acid encoding the Cas protein, and wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

In some such compositions or combinations, the Cas protein or the nucleic acid encoding the Cas protein and the guide RNA or the one or more DNAs encoding the guide RNA are associated with a lipid nanoparticle. In some such compositions or combinations, the lipid nanoparticle comprises a cationic lipid, a neutral lipid, a helper lipid, and a stealth lipid. In some such compositions or combinations, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or the neutral lipid is distearoylphosphatidylcholine or 1,2-distearoyl-sn-glycero-3-phosphocholine (DSPC), and/or the helper lipid is cholesterol, and/or the stealth lipid is 1,2-dimyristoyl-rac-glycero-3-methoxypolyethylene glycol-2000. In some such compositions or combinations, the cationic lipid is Lipid A, the neutral lipid is DSPC, the helper lipid is cholesterol, and the stealth lipid is PEG2k-DMG. In some such compositions or combinations, the lipid nanoparticle comprises four lipids at the following molar ratios: about 50 mol % Lipid A, about 9 mol % DSPC, about 38 mol % cholesterol, and about 3 mol % PEG2k-DMG.

Some such compositions or combinations are provided for use in a method of inserting a nucleic acid encoding a polypeptide of interest into a target genomic locus in a cell or a population of cells in a subject.

Some such compositions or combinations are provided for use in a method of expressing a polypeptide of interest from a target genomic locus in a cell or a population of cells in a subject.

Some such compositions or combinations are provided for use in a method of treating an enzyme deficiency in a subject in need thereof.

Some such compositions or combinations are provided for use in a method of preventing or reducing the onset of a sign or symptom of an enzyme deficiency in a subject in need thereof.

In another aspect, provided are kits comprising any such compositions or combinations described herein.

In another aspect, provided are plasma cell depleting agents for use in any such methods described herein.

In another aspect, provided are methods of inserting a nucleic acid encoding a polypeptide of interest into a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of an anti-CD20×CD3 bispecific antibody or functional fragment thereof, wherein the nuclease agent cleaves the nuclease target site, and the nucleic acid construct is inserted into the target genomic locus. In some such methods, provided are methods of inserting a nucleic acid encoding a polypeptide of interest into a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of an anti-CD20×CD3 bispecific antibody or functional fragment thereof, wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, and the nucleic acid construct is inserted into the target genomic locus.

In another aspect, provided are methods of expressing a polypeptide of interest from a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of an anti-CD20×CD3 bispecific antibody or functional fragment thereof, wherein the nuclease agent cleaves the nuclease target site, the nucleic acid construct is inserted into the target genomic locus to create a modified target genomic locus, and the polypeptide of interest is expressed from the modified target genomic locus. In some such methods, provided are methods of expressing a polypeptide of interest from a target genomic locus in a cell or a population of cells in a subject, comprising administering to the subject: (a) a nucleic acid construct comprising a coding sequence for the polypeptide of interest; (b) a nuclease agent or one or more nucleic acids encoding the nuclease agent, wherein the nuclease agent targets a nuclease target site in the target genomic locus; and (c) an effective amount of an anti-CD20×CD3 bispecific antibody or functional fragment thereof, wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, the nucleic acid construct is inserted into the target genomic locus to create a modified target genomic locus, and the polypeptide of interest is expressed from the modified target genomic locus.

In another aspect, provided are methods of treating an enzyme deficiency in a subject in need thereof, comprising administering to the subject: (a) a nucleic acid construct comprising a coding sequence for a polypeptide of interest, wherein the polypeptide of interest comprises an enzyme to treat the enzyme deficiency; (b) a nuclease agent or one or more nucleic acids encoding the nuclease agent, wherein the nuclease agent targets a nuclease target site in a target genomic locus; and (c) an effective amount of an anti-CD20×CD3 bispecific antibody or functional fragment thereof, wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency. In some such methods, provided are methods of treating an enzyme deficiency in a subject in need thereof, comprising administering to the subject: (a) a nucleic acid construct comprising a coding sequence for a polypeptide of interest, wherein the polypeptide of interest comprises an enzyme to treat the enzyme deficiency; (b) a nuclease agent or one or more nucleic acids encoding the nuclease agent, wherein the nuclease agent targets a nuclease target site in a target genomic locus; and (c) an effective amount of an anti-CD20×CD3 bispecific antibody or functional fragment thereof, wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency.

In another aspect, provided are methods of preventing or reducing the onset of a sign or symptom of an enzyme deficiency in a subject in need thereof, comprising administering to the subject: (a) a nucleic acid construct comprising a coding sequence for a polypeptide of interest, wherein the enzyme deficiency is characterized by a loss-of-function of the polypeptide of interest; (b) a nuclease agent or one or more nucleic acids encoding the nuclease agent, wherein the nuclease agent targets a nuclease target site in a target genomic locus; and (c) an effective amount of an anti-CD20×CD3 bispecific antibody or functional fragment thereof, wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency. In some such methods, provided are methods of preventing or reducing the onset of a sign or symptom of an enzyme deficiency in a subject in need thereof, comprising administering to the subject: (a) a nucleic acid construct comprising a coding sequence for a polypeptide of interest, wherein the enzyme deficiency is characterized by a loss-of-function of the polypeptide of interest; (b) a nuclease agent or one or more nucleic acids encoding the nuclease agent, wherein the nuclease agent targets a nuclease target site in a target genomic locus; and (c) an effective amount of an anti-CD20×CD3 bispecific antibody or functional fragment thereof, wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency.

In some such methods, the subject has a disease of a bleeding disorder characterized by the enzyme deficiency, a disease of an inborn error of metabolism characterized by the enzyme deficiency, or a lysosomal storage disease characterized by the enzyme deficiency. Optionally, the disease is hemophilia B and the polypeptide of interest is a factor IX protein, the disease is hemophilia A and the polypeptide of interest is a factor VIII protein, or the disease is Pompe disease and the polypeptide of interest is a multidomain therapeutic protein comprising a delivery domain fused to a lysosomal alpha-glucosidase.

Some such methods further comprise a subsequent administration step comprising administering to the subject at one or more subsequent times: (a) the nucleic acid construct; (b) the nuclease agent or the one or more nucleic acids encoding the nuclease agent; and optionally (c) the anti-CD20×CD3 bispecific antibody or functional fragment thereof, until a desired level of expression and/or activity of the polypeptide of interest is achieved in the subject.

Some such methods further comprise a subsequent administration step comprising administering to the subject at one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the anti-CD20×CD3 bispecific antibody or functional fragment thereof, until a desired level of expression and/or activity of the polypeptide of interest is achieved in the subject.

Some such methods further comprise a subsequent administration step comprising administering to the subject at one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus; and optionally (c) the anti-CD20×CD3 bispecific antibody or functional fragment thereof, until a desired level of expression and/or activity of the polypeptide of interest is achieved in the subject.

Some such methods further comprise a subsequent administration step comprising administering to the subject at one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus; and optionally (c) the anti-CD20×CD3 bispecific antibody or functional fragment thereof, until a desired level of expression and/or activity of the polypeptide of interest is achieved in the subject.

Some such methods further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest is achieved in the subject.

In some such methods, the polypeptide of interest is a factor IX protein, and the desired expression level of the factor IX protein in the subject is a serum level of at least about 3 μg/mL or about 3-5 μg/mL. In some such methods, the polypeptide of interest is a multidomain therapeutic protein comprising a delivery domain fused to a lysosomal alpha-glucosidase, and the desired expression level of the multidomain therapeutic protein in the subject is a serum level of at least about 2 μg/mL or at least about 5 μg/mL.

Some such methods further comprise a subsequent administration step comprising administering to the subject at one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus; and optionally (c) the anti-CD20×CD3 bispecific antibody or functional fragment thereof, wherein the second nuclease agent cleaves the second nuclease target site, and the second nucleic acid construct is inserted into the second target genomic locus.

In some such methods, the one or more subsequent administration steps is one subsequent administration step. In some such methods, the one or more subsequent administration steps is two subsequent administration steps or comprises at least two subsequent administration steps.

In some such methods, the anti-CD20×CD3 bispecific antibody or functional fragment thereof is administered in the one or more subsequent administration steps if there is no preexisting anti-CD20×CD3 bispecific antibody or functional fragment thereof in the subject or if the preexisting the expression and/or activity levels of the anti-CD20×CD3 bispecific antibody or functional fragment thereof are below a desired threshold level. Optionally, the method comprises measuring the expression and/or activity levels of the anti-CD20×CD3 bispecific antibody or functional fragment thereof prior to the one or more subsequent administration steps.

In some such methods, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45. In some such methods, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such methods, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45. In some such methods, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such methods, the anti-CD20×CD3 bispecific antibody or functional fragment thereof comprises: (a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and (b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

In some such methods, the anti-CD20×CD3 bispecific antibody or functional fragment thereof comprises a human IgG heavy chain constant region. Optionally, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such methods, the human IgG heavy chain constant region is isotype IgG4 or IgG1.

In some such methods, the anti-CD20×CD3 bispecific antibody or functional fragment thereof is administered simultaneously with the nucleic acid construct. In some such methods, the anti-CD20×CD3 bispecific antibody or functional fragment thereof is administered prior to the nucleic acid construct. In some such methods, the anti-CD20×CD3 bispecific antibody or functional fragment thereof is administered prior to and after the nucleic acid construct. In some such methods, the anti-CD20×CD3 bispecific antibody or functional fragment thereof is administered about 1 week prior to or within about 1 week prior to the nucleic acid construct. In some such methods, the nucleic acid construct is administered within about 3 months, within about 2 months, within about 7 weeks, within about 6 weeks, within about 5 weeks, within about 4 weeks, within about 3 weeks, within about 2 weeks, or within about 1 week after an initial dose of the anti-CD20×CD3 bispecific antibody or functional fragment thereof, or the nucleic acid construct is administered at least about 1 week, at least about 2 weeks, at least about 3 weeks, at least about 4 weeks, at least about 5 weeks, at least about 6 weeks, at least about 7 weeks, at least about 2 months, or at least about 3 months after an initial dose of the anti-CD20×CD3 bispecific antibody or functional fragment thereof. In some such methods, the nucleic acid construct is administered simultaneously with the nuclease agent or the one or more nucleic acids encoding the nuclease agent. In some such methods, the nucleic acid construct is administered prior to or after the nuclease agent or the one or more nucleic acids encoding the nuclease agent.

In some such methods, the nucleic acid construct is in the nucleic acid vector. Optionally, the nucleic acid vector is a viral vector. Optionally, the viral vector is administered at a dose of about 3E11 vg/kg to about 5E13 vg/kg. In some such methods, the nucleic acid vector is an adeno-associated viral (AAV) vector. Optionally, the nucleic acid construct is flanked by inverted terminal repeats (ITRs) on each end. Optionally, the ITR on at least one end comprises, consists essentially of, or consists of SEQ ID NO: 283. Optionally, the ITR on each end comprises, consists essentially of, or consists of SEQ ID NO: 283. Optionally, the ITR on at least one end comprises, consists essentially of, or consists of SEQ ID NO: 281. Optionally, the ITR on each end comprises, consists essentially of, or consists of SEQ ID NO: 281. In some such methods, the AAV vector is a single-stranded AAV (ssAAV) vector. In some such methods, the AAV vector is a recombinant AAV8 (rAAV8) vector.

In some such methods, the polypeptide of interest is a factor IX protein. In some such methods, the factor IX protein coding sequence encodes a factor IX protein comprising SEQ ID NO: 97. In some such methods, the factor IX protein coding sequence comprises or consists of SEQ ID NO: 68, or the factor IX protein coding sequence comprises or consists of SEQ ID NO: 61.

In some such methods, the nucleic acid construct is a bidirectional construct, wherein the factor IX protein coding sequence is a first factor IX protein coding sequence, and the bidirectional construct further comprises a reverse complement of a second factor IX protein coding sequence, wherein the first factor IX protein coding sequence and the second factor IX protein coding sequence are different but encode the same factor IX protein sequence. In some such methods, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 61 and the second factor IX protein coding sequence comprises SEQ ID NO: 68; or (ii) the first factor IX protein coding sequence comprises SEQ ID NO: 68 and the second factor IX protein coding sequence comprises SEQ ID NO: 61, wherein the nucleic acid construct does not comprise a promoter that drives the expression of the factor IX protein, and wherein the nucleic acid construct does not comprise homology arms. In some such methods, the nucleic acid construct comprises SEQ ID NO: 109 or 82 or the reverse complement thereof.

In some such methods, the nucleic acid construct is a unidirectional construct. In some such methods, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 61 or SEQ ID NO: 68, wherein the nucleic acid construct does not comprise a promoter that drives the expression of the factor IX protein, and wherein the nucleic acid construct does not comprise homology arms.

In some such methods, the polypeptide of interest is a multidomain therapeutic protein comprising a delivery domain fused to a lysosomal alpha-glucosidase. In some such methods, the lysosomal alpha-glucosidase comprises or consists of the sequence set forth in SEQ ID NO: 296. In some such methods, the lysosomal alpha-glucosidase coding sequence comprises or consist of the sequence set forth in SEQ ID NO: 857.

In some such methods, the delivery domain is a CD63-binding delivery domain. In some such methods, the CD63-binding delivery domain comprises an anti-CD63 antigen-binding protein. In some such methods, the CD63-binding delivery domain is a single-chain variable fragment (scFv). In some such methods, the scFv comprises or consists of the sequence set forth in SEQ ID NO: 306. In some such methods, the scFv coding sequence comprises or consists of the sequence set forth in SEQ ID NO: 866.

In some such methods, the multidomain therapeutic protein comprises or consists of the sequence set forth in SEQ ID NO: 316. In some such methods, the coding sequence for the multidomain therapeutic protein comprises or consists of the sequence set forth in SEQ ID NO: 863. Optionally, the nucleic acid construct comprises the sequence set forth in SEQ ID NO: 900 or 884. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 863. Optionally, the nucleic acid construct comprises the sequence set forth in SEQ ID NO: 900 or 884, wherein the polyadenylation signal comprises a BGH polyadenylation signal and a unidirectional SV40 late polyadenylation signal. Optionally, the BGH polyadenylation signal comprises the sequence set forth in SEQ ID NO: 858 and the unidirectional SV40 late polyadenylation signal comprises the sequence set forth in SEQ ID NO: 859. Optionally, the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such methods, the delivery domain is a TfR-binding delivery domain. In some such methods, the TfR-binding delivery domain comprises an anti-TfR antigen-binding protein. In some such methods, the anti-TfR antigen-binding protein comprises a HCVR comprising the HCDR1, HCDR2 and HCDR3 of a HCVR that comprises the amino acid sequence set forth in SEQ ID NO: 555 (or a variant thereof); and a LCVR comprising the LCDR1, LCDR2 and LCDR3 of a LCVR that comprises the amino acid sequence set forth in SEQ ID NO: 560 (or a variant thereof). In some such methods, the anti-TfR antigen-binding protein comprises a HCVR that comprises: an HCDR1 comprising the amino acid sequence set forth in SEQ ID NO: 556 (or a variant thereof), an HCDR2 comprising the amino acid sequence set forth in SEQ ID NO: 557 (or a variant thereof), and an HCDR3 comprising the amino acid sequence set forth in SEQ ID NO: 558 (or a variant thereof); and a LCVR that comprises: an LCDR1 comprising the amino acid sequence set forth in SEQ ID NO: 561 (or a variant thereof), an LCDR2 comprising the amino acid sequence set forth in SEQ ID NO: 562 (or a variant thereof), and an LCDR3 comprising the amino acid sequence set forth in SEQ ID NO: 563 (or a variant thereof). In some such methods, the anti-TfR antigen-binding protein comprises a HCVR that comprises the amino acid sequence set forth in SEQ ID NO: 555 (or a variant thereof); and a LCVR that comprises the amino acid sequence set forth in SEQ ID NO: 560 (or a variant thereof).

In some such methods, the TfR-binding delivery domain comprises a single-chain variable fragment (scFv). In some such methods, the scFv comprises or consists of the sequence set forth in SEQ ID NO: 672. In some such methods, the scFv coding sequence comprises or consists of the sequence set forth in SEQ ID NO: 713.

In some such methods, the multidomain therapeutic protein comprises or consists of the sequence set forth in SEQ ID NO: 691. In some such methods, the coding sequence for the multidomain therapeutic protein comprises or consists of the sequence set forth in SEQ ID NO: 852. Optionally, the nucleic acid construct comprises the sequence set forth in SEQ ID NO: 887 or 871. In some such methods, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 852. Optionally, the nucleic acid construct comprises the sequence set forth in SEQ ID NO: 887 or 871, wherein the polyadenylation signal comprises a BGH polyadenylation signal and a unidirectional SV40 late polyadenylation signal. Optionally, the BGH polyadenylation signal comprises the sequence set forth in SEQ ID NO: 858 and the unidirectional SV40 late polyadenylation signal comprises the sequence set forth in SEQ ID NO: 859. Optionally, the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such methods, the polypeptide of interest is a factor VIII protein.

In some such methods, the polypeptide of interest is an antigen-binding protein. Optionally, the antigen-binding protein is an antibody.

In some such methods, the target genomic locus is an albumin gene. Optionally, the albumin gene is a human albumin gene. In some such methods, the nuclease target site is in intron 1 of the albumin gene.

In some such methods, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such methods, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such methods, the DNA-targeting segment comprises any one of SEQ ID NOS: 153-184. Optionally, the DNA-targeting segment comprises any one of SEQ ID NOS: 159, 153, 156, and 164, or the DNA-targeting segment consists of any one of SEQ ID NOS: 153-184. Optionally, the DNA-targeting segment consists of any one of SEQ ID NOS: 159, 153, 156, and 164. In some such methods, the guide RNA comprises any one of SEQ ID NOS: 185-248. Optionally, the guide RNA comprises any one of SEQ ID NOS: 191, 223, 185, 217, 188, 220, 196, and 228. In some such methods, the DNA-targeting segment comprises or consists of SEQ ID NO: 159. In some such methods, the guide RNA comprises SEQ ID NO: 191 or 223.

Some such methods comprise administering the guide RNA in the form of RNA. In some such methods, the guide RNA comprises at least one modification. In some such methods, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. Some such methods comprise administering the guide RNA in the form of RNA, wherein the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such methods, the Cas protein is a Cas9 protein. Optionally, the Cas protein is derived from a Streptococcus pyogenes Cas9 protein. In some such methods, the Cas protein comprises the sequence set forth in SEQ ID NO: 134.

Some such methods comprise administering the nucleic acid encoding the Cas protein, wherein the nucleic acid comprises an mRNA encoding the Cas protein. In some such methods, the mRNA encoding the Cas protein comprises at least one modification. In some such methods, the mRNA encoding the Cas protein is fully substituted with N1-methyl-pseudouridine. In some such methods, the mRNA encoding the Cas protein comprises the sequence set forth in SEQ ID NO: 124 or 125. Some such methods comprise administering the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

Some such methods comprise administering the guide RNA in the form of RNA, and the guide RNA comprises SEQ ID NO: 191 or 223, and also comprise administering the nucleic acid encoding the Cas protein, wherein the nucleic acid comprises an mRNA encoding the Cas protein, and the mRNA encoding the Cas protein comprises the sequence set forth in SEQ ID NO: 124 or 125. Some such methods comprise administering the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and also comprise administering the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

In some such methods, the Cas protein or the nucleic acid encoding the Cas protein and the guide RNA or the one or more DNAs encoding the guide RNA are associated with a lipid nanoparticle. In some such methods, the lipid nanoparticle comprises a cationic lipid, a neutral lipid, a helper lipid, and a stealth lipid. In some such methods, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or the neutral lipid is distearoylphosphatidylcholine or 1,2-distearoyl-sn-glycero-3-phosphocholine (DSPC), and/or the helper lipid is cholesterol, and/or the stealth lipid is 1,2-dimyristoyl-rac-glycero-3-methoxypolyethylene glycol-2000. In some such methods, the cationic lipid is Lipid A, the neutral lipid is DSPC, the helper lipid is cholesterol, and the stealth lipid is PEG2k-DMG. In some such methods, the lipid nanoparticle comprises four lipids at the following molar ratios: about 50 mol % Lipid A, about 9 mol % DSPC, about 38 mol % cholesterol, and about 3 mol % PEG2k-DMG.

In some such methods, the cell is a liver cell or a hepatocyte, or the population of cells is a population of liver cells or hepatocytes. In some such methods, the subject is a human subject. In some such methods, the subject is a neonatal subject. In some such methods, the nucleic acid vector is in an adeno-associated viral (AAV) vector, and the subject does not have preexisting AAV immunity.

In some such methods, the method does not comprise administering a plasma cell depleting agent. In some such methods, the nucleic acid vector is in an adeno-associated viral (AAV) vector, the subject does not have preexisting AAV immunity, and the method does not comprise administering a plasma cell depleting agent.

In another aspect, provided are compositions or combinations comprising an effective amount of an anti-CD20×CD3 bispecific antibody or functional fragment thereof in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus.

In some such compositions or combinations, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45. In some such compositions or combinations, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such compositions or combinations, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45. In some such compositions or combinations, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some such compositions or combinations, the anti-CD20×CD3 bispecific antibody or functional fragment thereof comprises: (a) a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and (b) a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

In some such compositions or combinations, the anti-CD20×CD3 bispecific antibody or functional fragment thereof comprises a human IgG heavy chain constant region. Optionally, the human IgG heavy chain constant region comprises one or more modifications that increase binding to a neonatal Fc receptor (FcRn) and/or the human IgG heavy chain constant region comprises one or more modifications that decrease binding to an Fc-gamma receptor (FcγR). In some such compositions or combinations, the human IgG heavy chain constant region is isotype IgG4 or IgG1.

In some such compositions or combinations, the nucleic acid construct is in the nucleic acid vector. Optionally, the nucleic acid vector is a viral vector. In some such compositions or combinations, the nucleic acid vector is an adeno-associated viral (AAV) vector. Optionally, the nucleic acid construct is flanked by inverted terminal repeats (ITRs) on each end. Optionally, the ITR on at least one end comprises, consists essentially of, or consists of SEQ ID NO: 283. Optionally, the ITR on each end comprises, consists essentially of, or consists of SEQ ID NO: 283. Optionally, the ITR on at least one end comprises, consists essentially of, or consists of SEQ ID NO: 281. Optionally, the ITR on each end comprises, consists essentially of, or consists of SEQ ID NO: 281. In some such compositions or combinations, the AAV vector is a single-stranded AAV (ssAAV) vector. In some such compositions or combinations, the AAV vector is a recombinant AAV8 (rAAV8) vector.

In some such compositions or combinations, the polypeptide of interest is a factor IX protein. In some such compositions or combinations, the factor IX protein coding sequence encodes a factor IX protein comprising SEQ ID NO: 97. In some such compositions or combinations, the factor IX protein coding sequence comprises or consists of SEQ ID NO: 68, or the factor IX protein coding sequence comprises or consists of SEQ ID NO: 61.

In some such compositions or combinations, the nucleic acid construct is a bidirectional construct, wherein the factor IX protein coding sequence is a first factor IX protein coding sequence, and the bidirectional construct further comprises a reverse complement of a second factor IX protein coding sequence, wherein the first factor IX protein coding sequence and the second factor IX protein coding sequence are different but encode the same factor IX protein sequence. In some such compositions or combinations, the nucleic acid construct comprises from 5′ to 3′: a first splice acceptor, the first factor IX protein coding sequence, a first polyadenylation signal, a reverse complement of a second polyadenylation signal, the reverse complement of the second factor IX protein coding sequence, and a reverse complement of a second splice acceptor, wherein: (i) the first factor IX protein coding sequence comprises SEQ ID NO: 61 and the second factor IX protein coding sequence comprises SEQ ID NO: 68; or (ii) the first factor IX protein coding sequence comprises SEQ ID NO: 68 and the second factor IX protein coding sequence comprises SEQ ID NO: 61, wherein the nucleic acid construct does not comprise a promoter that drives the expression of the factor IX protein, and wherein the nucleic acid construct does not comprise homology arms. In some such compositions or combinations, the nucleic acid construct comprises SEQ ID NO: 109 or 82 or the reverse complement thereof.

In some such compositions or combinations, the nucleic acid construct is a unidirectional construct. In some such compositions or combinations, the nucleic acid construct is a unidirectional construct comprising the factor IX protein coding sequence, wherein the nucleic acid construct comprises from 5′ to 3′: a splice acceptor, the factor IX protein coding sequence, and a polyadenylation signal, wherein the factor IX protein coding sequence comprises SEQ ID NO: 61 or SEQ ID NO: 68, wherein the nucleic acid construct does not comprise a promoter that drives the expression of the factor IX protein, and wherein the nucleic acid construct does not comprise homology arms.

In some such compositions or combinations, the polypeptide of interest is a multidomain therapeutic protein comprising a delivery domain fused to a lysosomal alpha-glucosidase. In some such compositions or combinations, the lysosomal alpha-glucosidase comprises or consists of the sequence set forth in SEQ ID NO: 296. In some such compositions or combinations, the lysosomal alpha-glucosidase coding sequence comprises or consists of the sequence set forth in SEQ ID NO: 857.

In some such compositions or combinations, the delivery domain is a CD63-binding delivery domain. In some such compositions or combinations, the CD63-binding delivery domain comprises an anti-CD63 antigen-binding protein. In some such compositions or combinations, the CD63-binding delivery domain is a single-chain variable fragment (scFv). In some such compositions or combinations, the scFv comprises or consists of the sequence set forth in SEQ ID NO: 306. In some such compositions or combinations, the scFv coding sequence comprises or consists of the sequence set forth in SEQ ID NO: 866.

In some such compositions or combinations, the multidomain therapeutic protein comprises or consists of the sequence set forth in SEQ ID NO: 316. In some such compositions or combinations, the coding sequence for the multidomain therapeutic protein comprises or consists of the sequence set forth in SEQ ID NO: 863. Optionally, the nucleic acid construct comprises the sequence set forth in SEQ ID NO: 900 or 884. In some such compositions or combinations, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 863. Optionally, the nucleic acid construct comprises the sequence set forth in SEQ ID NO: 900 or 884, wherein the polyadenylation signal comprises a BGH polyadenylation signal and a unidirectional SV40 late polyadenylation signal. Optionally, the BGH polyadenylation signal comprises the sequence set forth in SEQ ID NO: 858 and the unidirectional SV40 late polyadenylation signal comprises the sequence set forth in SEQ ID NO: 859. Optionally, the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such compositions or combinations, the delivery domain is a TfR-binding delivery domain. In some such compositions or combinations, the TfR-binding delivery domain comprises an anti-TfR antigen-binding protein. In some such compositions or combinations, the anti-TfR antigen-binding protein comprises a HCVR comprising the HCDR1, HCDR2 and HCDR3 of a HCVR that comprises the amino acid sequence set forth in SEQ ID NO: 555 (or a variant thereof); and a LCVR comprising the LCDR1, LCDR2 and LCDR3 of a LCVR that comprises the amino acid sequence set forth in SEQ ID NO: 560 (or a variant thereof). In some such compositions or combinations, the anti-TfR antigen-binding protein comprises a HCVR that comprises: an HCDR1 comprising the amino acid sequence set forth in SEQ ID NO: 556 (or a variant thereof), an HCDR2 comprising the amino acid sequence set forth in SEQ ID NO: 557 (or a variant thereof), and an HCDR3 comprising the amino acid sequence set forth in SEQ ID NO: 558 (or a variant thereof); and a LCVR that comprises: an LCDR1 comprising the amino acid sequence set forth in SEQ ID NO: 561 (or a variant thereof), an LCDR2 comprising the amino acid sequence set forth in SEQ ID NO: 562 (or a variant thereof), and an LCDR3 comprising the amino acid sequence set forth in SEQ ID NO: 563 (or a variant thereof). In some such compositions or combinations, the anti-TfR antigen-binding protein comprises a HCVR that comprises the amino acid sequence set forth in SEQ ID NO: 555 (or a variant thereof); and a LCVR that comprises the amino acid sequence set forth in SEQ ID NO: 560 (or a variant thereof).

In some such compositions or combinations, the TfR-binding delivery domain comprises a single-chain variable fragment (scFv). In some such compositions or combinations, the scFv comprises or consists of the sequence set forth in SEQ ID NO: 672. In some such compositions or combinations, the scFv coding sequence comprises or consists of the sequence set forth in SEQ ID NO: 713.

In some such compositions or combinations, the multidomain therapeutic protein comprises or consists of the sequence set forth in SEQ ID NO: 691. In some such compositions or combinations, the coding sequence for the multidomain therapeutic protein comprises or consists of the sequence set forth in SEQ ID NO: 852. Optionally, the nucleic acid construct comprises the sequence set forth in SEQ ID NO: 887 or 871. In some such compositions or combinations, the nucleic acid construct comprises from 5′ to 3′: a splice acceptor, the coding sequence for the multidomain therapeutic protein, and a polyadenylation signal or sequence, wherein the coding sequence for the multidomain therapeutic protein comprises the sequence set forth in SEQ ID NO: 852. Optionally, the nucleic acid construct comprises the sequence set forth in SEQ ID NO: 887 or 871, wherein the polyadenylation signal comprises a BGH polyadenylation signal and a unidirectional SV40 late polyadenylation signal. Optionally, the BGH polyadenylation signal comprises the sequence set forth in SEQ ID NO: 858 and the unidirectional SV40 late polyadenylation signal comprises the sequence set forth in SEQ ID NO: 859. Optionally, the polyadenylation signal comprising the BGH polyadenylation signal and the unidirectional SV40 late polyadenylation signal comprises the sequence set forth in SEQ ID NO: 902, wherein the nucleic acid construct does not comprise a promoter that drives the expression of the multidomain therapeutic protein, and wherein the nucleic acid construct does not comprise a homology arm.

In some such compositions or combinations, the polypeptide of interest is a factor VIII protein.

In some such compositions or combinations, the polypeptide of interest is an antigen-binding protein. Optionally, the antigen-binding protein is an antibody.

In some such compositions or combinations, the target genomic locus is an albumin gene. Optionally, the albumin gene is a human albumin gene. In some such compositions or combinations, the nuclease target site is in intron 1 of the albumin gene.

In some such compositions or combinations, the nuclease agent comprises: (a) a zinc finger nuclease (ZFN); (b) a transcription activator-like effector nuclease (TALEN); or (c) (i) a Cas protein or a nucleic acid encoding the Cas protein; and (ii) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence.

In some such compositions or combinations, the nuclease agent comprises: (a) a Cas protein or a nucleic acid encoding the Cas protein; and (b) a guide RNA or one or more DNAs encoding the guide RNA, wherein the guide RNA comprises a DNA-targeting segment that targets a guide RNA target sequence, and wherein the guide RNA binds to the Cas protein and targets the Cas protein to the guide RNA target sequence. In some such compositions or combinations, the DNA-targeting segment comprises any one of SEQ ID NOS: 153-184. Optionally, the DNA-targeting segment comprises any one of SEQ ID NOS: 159, 153, 156, and 164, or the DNA-targeting segment consists of any one of SEQ ID NOS: 153-184. Optionally, the DNA-targeting segment consists of any one of SEQ ID NOS: 159, 153, 156, and 164. In some such compositions or combinations, the guide RNA comprises any one of SEQ ID NOS: 185-248. Optionally, the guide RNA comprises any one of SEQ ID NOS: 191, 223, 185, 217, 188, 220, 196, and 228. In some such compositions or combinations, the DNA-targeting segment comprises or consists of SEQ ID NO: 159. In some such compositions or combinations, the guide RNA comprises SEQ ID NO: 191 or 223.

Some such compositions or combinations comprise the guide RNA in the form of RNA. In some such compositions or combinations, the guide RNA comprises at least one modification. In some such compositions or combinations, the at least one modification comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA. Some such compositions or combinations comprise the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA.

In some such compositions or combinations, the Cas protein is a Cas9 protein. Optionally, the Cas protein is derived from a Streptococcus pyogenes Cas9 protein. In some such compositions or combinations, the Cas protein comprises the sequence set forth in SEQ ID NO: 134.

Some such compositions or combinations comprise the nucleic acid encoding the Cas protein, wherein the nucleic acid comprises an mRNA encoding the Cas protein. In some such compositions or combinations, the mRNA encoding the Cas protein comprises at least one modification. In some such compositions or combinations, the mRNA encoding the Cas protein is fully substituted with N1-methyl-pseudouridine. In some such compositions or combinations, the mRNA encoding the Cas protein comprises the sequence set forth in SEQ ID NO: 124 or 125. Some such compositions or combinations comprise the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

Some such compositions or combinations comprise the guide RNA in the form of RNA, and the guide RNA comprises SEQ ID NO: 191 or 223, and the composition or combination comprises administering the nucleic acid encoding the Cas protein, wherein the nucleic acid comprises an mRNA encoding the Cas protein, and the mRNA encoding the Cas protein comprises the sequence set forth in SEQ ID NO: 124 or 125. Some such compositions or combinations comprise the guide RNA in the form of RNA, the guide RNA comprises SEQ ID NO: 223, and the guide RNA comprises: (i) phosphorothioate bonds between the first four nucleotides at the 5′ end of the guide RNA; (ii) phosphorothioate bonds between the last four nucleotides at the 3′ end of the guide RNA; (iii) 2′-O-methyl-modified nucleotides at the first three nucleotides at the 5′ end of the guide RNA; and (iv) 2′-O-methyl-modified nucleotides at the last three nucleotides at the 3′ end of the guide RNA, and the composition or combination comprises the nucleic acid encoding the Cas protein, wherein the nucleic acid comprises an mRNA encoding the Cas protein, the mRNA encoding the Cas protein comprises the sequence set forth in SEQ ID NO: 124 or 125, and the mRNA encoding the Cas protein is fully substituted with N1-methyl-pseudouridine, comprises a 5′ cap, and comprises a poly(A) tail.

In some such compositions or combinations, the Cas protein or the nucleic acid encoding the Cas protein and the guide RNA or the one or more DNAs encoding the guide RNA are associated with a lipid nanoparticle. In some such compositions or combinations, the lipid nanoparticle comprises a cationic lipid, a neutral lipid, a helper lipid, and a stealth lipid. In some such compositions or combinations, the cationic lipid is Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate), and/or the neutral lipid is distearoylphosphatidylcholine or 1,2-distearoyl-sn-glycero-3-phosphocholine (DSPC), and/or the helper lipid is cholesterol, and/or the stealth lipid is 1,2-dimyristoyl-rac-glycero-3-methoxypolyethylene glycol-2000. In some such compositions or combinations, the cationic lipid is Lipid A, the neutral lipid is DSPC, the helper lipid is cholesterol, and the stealth lipid is PEG2k-DMG. In some such compositions or combinations, the lipid nanoparticle comprises four lipids at the following molar ratios: about 50 mol % Lipid A, about 9 mol % DSPC, about 38 mol % cholesterol, and about 3 mol % PEG2k-DMG.

In some such compositions or combinations, the composition or combination does not comprise a plasma cell depleting agent.

In another aspect, the compositions or combinations described herein are provided for use in a method of inserting a nucleic acid encoding a polypeptide of interest into a target genomic locus in a cell or a population of cells in a subject.

In another aspect, the compositions or combinations described herein are provided for use in a method of expressing a polypeptide of interest from a target genomic locus in a cell or a population of cells in a subject.

In another aspect, the compositions or combinations described herein are provided for use in a method of treating an enzyme deficiency in a subject in need thereof.

In another aspect, the compositions or combinations described herein are provided for use in a method of preventing or reducing the onset of a sign or symptom of an enzyme deficiency in a subject in need thereof.

In another aspect, provided are kits comprising the compositions or combinations described herein.

In another aspect, provided is an anti-CD20×CD3 bispecific antibody or functional fragment thereof for use in the methods described herein.

### BRIEF DESCRIPTION OF THE FIGURES

FIG. 1 shows an experimental timeline for the study described in Examples 1, 2, 3, and 11.

FIG. 2 shows the effect of plasma cell depletion with anti-BCMA×CD3 bispecific antibody, FcRn blockade via efgartigimod alfa, B cell depletion with anti-CD19 and anti-CD20 antibodies (anti-CD19/CD20 antibodies), or combination thereof, on anti-AAV8 capsid IgG titers over time in mice previously treated with recombinant AAV8 vector.

FIG. 3 shows the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second AAV8 vector in mice previously treated with recombinant AAV8 vector, as measured by Taqman quantitative real-time polymerase chain reaction (PCR) of green fluorescent protein (GFP) transgene DNA.

FIG. 4 shows the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second recombinant AAV8 vector in mice previously treated with a first recombinant AAV8 vector, as measured by Taqman quantitative real-time reverse-transcription PCR of GFP transgene RNA.

FIGS. 5A-5B show the effect of plasma cell depletion with anti-BCMA×CD3 bispecific antibody, FcRn blockade via efgartigimod alfa, B cell depletion with anti-CD19/CD20 antibodies, or combination thereof, on liver transduction 10 days following administration of a second recombinant AAV8 vector in mice previously treated with a first recombinant AAV8 vector, as measured by GFP immunohistochemical (IHC) staining of formalin-fixed paraffin embedded liver sections. FIG. 5A shows GFP-positive area quantified using HALO software (Indica labs). FIG. 5B shows representative images.

FIGS. 6A-6J show flow cytometry analysis of B cell and plasma cell frequencies and counts in bone marrow and spleen following treatment with anti-BCMA×CD3 bispecific antibody, FcRn blockade, anti-CD19/CD20 antibodies, or combinations thereof. FIG. 6A shows bone marrow plasma cell frequencies, FIG. 6B shows spleen plasma cell frequencies, FIG. 6C shows spleen naïve B cell frequencies, FIG. 6D shows spleen total memory B cell frequencies, FIG. 6E shows spleen AAV-specific memory B cell frequencies, FIG. 6F shows bone marrow plasma cell counts, FIG. 6G shows spleen plasma cell counts, FIG. 6H shows spleen naïve B cell counts, FIG. 6I shows spleen total memory B cell counts, and FIG. 6J shows spleen AAV-specific memory B cell counts.

FIG. 7 shows the effect of efgartigimod on serum drug concentration of REGN5458 (BCMA×CD3).

FIG. 8 shows an experimental timeline for the study described in Example 12.

FIGS. 9A-9B show the effect of plasma cell depletion, B cell depletion, neonatal Fc receptor blockade, and combinations thereof, on naturally-occurring anti-AAV antibody titers in cynomolgus macaques. AAV8 neutralizing antibody (NAb) titer levels are presented for each treatment group over the duration of the study (FIG. 9A) and specifically at Study Day 29 (FIG. 9B).

FIG. 10 shows an experimental timeline for the study described in Examples 13 and 14.

FIGS. 11A-11C show a comparison of the effect of CD20×CD3-mediated versus anti-CD20-mediated B cell depletion on the development of anti-AAV IgM antibody titers (FIG. 11A) and anti-AAV IgG antibody titers (FIGS. 11B-11C) in mice.

FIGS. 12A-12C show a comparison of the effect of CD20×CD3-mediated versus anti-CD20-mediated B cell depletion on AAV transduction (FIG. 12A) and transgene expression (FIGS. 12B-12C) following vector re-administration in mice.

FIG. 13 shows an experimental timeline for the study described in Examples 15 and 16.

FIGS. 14A-14F show the effect of prophylactic CD20×CD3-mediated B cell depletion on serum anti-AAV8 IgM (FIG. 14A and FIG. 14D), IgG (FIG. 14B and FIG. 14E), and neutralizing antibody (nAb) (FIG. 14C and FIG. 14F) titers in cynomolgus macaques.

FIGS. 15A-15C show the effect of prophylactic CD20×CD3-mediated B cell depletion on AAV transduction (FIG. 15A) and transgene expression (FIGS. 15B-15C) following AAV vector re-administration in cynomolgus macaques.

### DEFINITIONS

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones. The term “domain” refers to any part of a protein or polypeptide having a particular function or structure.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

The term “expression vector” or “expression construct” or “expression cassette” refers to a recombinant nucleic acid containing a desired coding sequence operably linked to appropriate nucleic acid sequences necessary for the expression of the operably linked coding sequence in a particular host cell or organism. Nucleic acid sequences necessary for expression in prokaryotes usually include a promoter, an operator (optional), and a ribosome binding site, as well as other sequences. Eukaryotic cells are generally known to utilize promoters, enhancers, and termination and polyadenylation signals, although some elements may be deleted and other elements added without sacrificing the necessary expression.

The term “viral vector” refers to a recombinant nucleic acid that includes at least one element of viral origin and includes elements sufficient for or permissive of packaging into a viral vector particle. The vector and/or particle can be utilized for the purpose of transferring DNA, RNA, or other nucleic acids into cells either ex vivo or in vivo. Numerous forms of viral vectors are known.

The term “isolated” with respect to proteins, nucleic acids, and cells includes proteins, nucleic acids, and cells that are relatively purified with respect to other cellular or organism components that may normally be present in situ, up to and including a substantially pure preparation of the protein, nucleic acid, or cell. The term “isolated” may include proteins and nucleic acids that have no naturally occurring counterpart or proteins or nucleic acids that have been chemically synthesized and are thus substantially uncontaminated by other proteins or nucleic acids. The term “isolated” may include proteins, nucleic acids, or cells that have been separated or purified from most other cellular components or organism components with which they are naturally accompanied (e.g., but not limited to, other cellular proteins, nucleic acids, or cellular or extracellular components).

The term “wild type” or “wild-type” includes entities having a structure and/or activity as found in a normal (as contrasted with mutant, diseased, altered, or so forth) state or context. Wild type genes and polypeptides often exist in multiple different forms (e.g., alleles).

The term “endogenous sequence” refers to a nucleic acid sequence that occurs naturally within a cell or animal. For example, an endogenous ALB sequence of an animal refers to a native ALB sequence that naturally occurs at the ALB locus in the animal.

“Exogenous” molecules or sequences include molecules or sequences that are not normally present in a cell in that form or that are introduced into a cell from an outside source. Normal presence includes presence with respect to the particular developmental stage and environmental conditions of the cell. An exogenous molecule or sequence, for example, can include a mutated version of a corresponding endogenous sequence within the cell, such as a humanized version of the endogenous sequence, or can include a sequence corresponding to an endogenous sequence within the cell but in a different form (i.e., not within a chromosome). In contrast, endogenous molecules or sequences include molecules or sequences that are normally present in that form in a particular cell at a particular developmental stage under particular environmental conditions.

The term “heterologous” when used in the context of a nucleic acid or a protein indicates that the nucleic acid or protein comprises at least two segments that do not naturally occur together in the same molecule. For example, the term “heterologous,” when used with reference to segments of a nucleic acid or segments of a protein, indicates that the nucleic acid or protein comprises two or more sub-sequences that are not found in the same relationship to each other (e.g., joined together) in nature. As one example, a “heterologous” region of a nucleic acid vector is a segment of nucleic acid within or attached to another nucleic acid molecule that is not found in association with the other molecule in nature. For example, a heterologous region of a nucleic acid vector could include a coding sequence flanked by a heterologous promoter not found in association with the coding sequence in nature. Likewise, a “heterologous” region of a protein is a segment of amino acids within or attached to another peptide molecule that is not found in association with the other peptide molecule in nature (e.g., a fusion protein, or a protein with a tag). Similarly, a nucleic acid or protein can comprise a heterologous label or a heterologous secretion or localization sequence.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells by replacing at least one codon of the native sequence with a codon that is more frequently or most frequently used in the genes of the host cell while maintaining the native amino acid sequence. For example, a nucleic acid encoding a protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host are also available (see, e.g., Gene Forge).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). Examples of promoters can be found, for example, in WO 2013/176772, herein incorporated by reference in its entirety for all purposes.

A constitutive promoter is one that is active in all tissues or particular tissues at all developing stages. Examples of constitutive promoters include the human cytomegalovirus immediate early (hCMV), mouse cytomegalovirus immediate early (mCMV), human elongation factor 1 alpha (hEF1α), mouse elongation factor 1 alpha (mEF1α), mouse phosphoglycerate kinase (PGK), chicken beta actin hybrid (CAG or CBh), SV40 early, and beta 2 tubulin promoters.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline (tet)-regulated promoters (e.g., a tetracycline-responsive promoter, a tetracycline operator sequence (tetO), a tet-On promoter, or a tet-Off promoter), steroid-regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters or glial-specific promoters or muscle-specific promoters or liver-specific promoters.

Developmentally regulated promoters include, for example, promoters active only during an embryonic stage of development, or only in an adult cell.

“Operable linkage” or being “operably linked” includes juxtaposition of two or more components (e.g., a promoter and another sequence element) such that both components function normally and allow the possibility that at least one of the components can mediate a function that is exerted upon at least one of the other components. For example, a promoter can be operably linked to a coding sequence if the promoter controls the level of transcription of the coding sequence in response to the presence or absence of one or more transcriptional regulatory factors. Operable linkage can include such sequences being contiguous with each other or acting in trans (e.g., a regulatory sequence can act at a distance to control transcription of the coding sequence).

The term “in vitro” includes artificial environments and to processes or reactions that occur within an artificial environment (e.g., a test tube or an isolated cell or cell line). The term “in vivo” includes natural environments (e.g., a cell, organism, or body) and to processes or reactions that occur within a natural environment. The term “ex vivo” includes cells that have been removed from the body of an individual and processes or reactions that occur within such cells.

The term “antigen-binding molecule” includes antibodies and antigen-binding fragments of antibodies, including multispecific antibodies, e.g., bispecific antibodies.

The term “antibody,” as used herein, refers to an antigen-binding molecule or molecular complex comprising a set of complementarity determining regions (CDRs) that specifically bind to or interact with a particular antigen (e.g., BCMA, CD20, CD3). The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the antibody (or antigen-binding portion thereof) may be identical to the human germline sequences, or may be naturally or artificially modified. An amino acid consensus sequence may be defined based on a side-by-side analysis of two or more CDRs.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, but are not limited to, the Kabat definition, the Chothia definition, the AbM definition (enhanced Chothia or Martin), the IMGT definition, and the Honneger definition (AHo). In general terms, the Kabat definition is based on sequence variability, the Chothia definition is based on the location of the structural loop regions, and the AbM definition is a compromise between the Kabat and Chothia approaches. See, e.g., Kabat et al., “Sequences of Proteins of Immunological Interest,” National Institutes of Health, Bethesda, Md. (1991); Chothia et al., J Mol Biol (1987), 4:901-17; Al-Lazikani et al., J. Mol. Biol. 273:927-948 (1997); and Martin et al., Proc. Natl. Acad. Sci. USA 86:9268-9272 (1989); see also, Dondelinger et al., Front. Immunol. (2018), 9:2278, doi:10.3389/fimmu.2018.02278. Public databases are also available for identifying CDR sequences within an antibody.

The term “antibody,” as used herein, also includes antigen-binding fragments of full antibody molecules. The terms “antigen-binding portion” of an antibody, “antigen-binding fragment” of an antibody, “antigen-binding domain,” and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add, or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody include: (i) V_{H}-C_{H}1; (ii) V_{H}-C_{H}2; (iii) V_{H}-C_{H}3; (iv) V_{H}-C_{H}1-C_{H}2; (v) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen.

Any multispecific antibody format may be adapted for use in the context of an antibody or antigen-binding fragment of an antibody of the present disclosure using routine techniques available in the art. For example, the present disclosure includes bispecific antibodies wherein one arm of an immunoglobulin is specific for an epitope of BCMA or CD20 and the other arm of the immunoglobulin is specific for an epitope of CD3. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency, and geometry. See, e.g., Kazane et al., J. Am. Chem. Soc. (Epub: Dec. 4, 2012).

The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The term “recombinant antibody,” as used herein, is intended to include all antibodies that are prepared, expressed, created, or isolated by recombinant means. The term includes, but is not limited to, antibodies expressed using a recombinant expression vector transfected into a host cell (e.g., Chinese hamster ovary (CHO) cell) or cellular expression system, antibodies isolated from a recombinant, combinatorial human antibody library, and antibodies isolated from a non-human animal (e.g., a mouse, such as a mouse that is transgenic for human immunoglobulin genes (see, e.g., Taylor et al. (1992) Nucl. Acids Res. 20:6287-6295). In some embodiments, the recombinant antibody is a recombinant human antibody. In some embodiments, recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Specific binding can be characterized by an equilibrium dissociation constant of at least about 1×10⁻⁶ M or less, e.g., 10⁻⁷ M, 10⁻⁸ M, 10⁻⁹ M, 10⁻¹⁰ M, 10⁻¹¹ M, or 10⁻¹² M (a smaller K_{D} denotes a tighter binding). Methods for determining whether an antibody specifically binds to an antigen are known in the art and include, for example, equilibrium dialysis, surface plasmon resonance (e.g., BIACORE™), bio-layer interferometry assay (e.g., Octet® HTX biosensor), solution-affinity ELISA, and the like. In some embodiments, specific binding is measured in a surface plasmon resonance assay, e.g., at 25° C. or 37° C. An antibody or antigen-binding fragment that specifically binds an antigen from one species may or may not have cross-reactivity to other antigens, such as an orthologous antigen from another species.

The term “K_{D},” as used herein, refers to the equilibrium dissociation constant of a particular antibody-antigen interaction.

The term “surface plasmon resonance,” as used herein, refers to an optical phenomenon that allows for the analysis of real-time biomolecular interactions by detection of alterations in protein concentrations within a biosensor matrix, for example using the BIACORE™ system (Cytiva, Marlborough, MA).

The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. The term “epitope” also refers to a site on an antigen to which B and/or T cells respond. It also refers to a region of an antigen that is bound by an antibody. Epitopes may be either linear or discontinuous (e.g., conformational). A linear epitope is one produced by adjacent amino acid residues in a polypeptide chain. A conformational epitope is produced by spatially juxtaposed amino acids from different segments of the linear polypeptide chain. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes may also be defined as structural or functional. Functional epitopes are generally a subset of the structural epitopes and have those residues that directly contribute to the affinity of the interaction. An epitope typically includes at least 3, and more usually, e.g., at least 5 or at least 8-10 amino acids in a unique spatial conformation.

Methods for determining the epitope of an antigen-binding protein, e.g., an antibody or antigen-binding fragment, include alanine scanning mutational analysis, peptide blot analysis (Reineke, Methods Mol Biol 2004, 248:443-463), peptide cleavage analysis, crystallographic studies, and nuclear magnetic resonance (NMR) analysis. In addition, methods such as epitope exclusion, epitope extraction, and chemical modification of antigens can be employed (Tomer, Prot Sci 2000, 9:487-496). Another method that can be used to identify the amino acids within a polypeptide with which an antigen-binding protein (e.g., an antibody or antigen-binding fragment) interacts is hydrogen/deuterium exchange detected by mass spectrometry (HDX). See, e.g., Ehring, Analytical Biochemistry 1999, 267:252-259; Engen and Smith, Anal Chem 2001, 73:256A-265A.

The term “competes,” as used in reference to competing for binding, refers to an antigen-binding protein (e.g., antibody or antigen-binding fragment) that binds to an antigen and inhibits or blocks the binding of another antigen-binding protein (e.g., antibody or antigen-binding fragment) to the antigen. Unless otherwise stated, the term also includes competition between two antigen-binding proteins (e.g., antibodies) in both orientations, i.e., a first antigen that binds an antigen and blocks binding of the antigen by a second antibody, and vice versa. Thus, in some embodiments, competition occurs in one such orientation. In some embodiments, the first antigen-binding protein (e.g., antibody) and second antigen-binding protein (e.g., antibody) may bind to the same epitope. Alternatively, the first and second antigen-binding proteins (e.g., antibodies) may bind to different epitopes, which may be overlapping or non-overlapping, wherein binding of one antigen-binding protein inhibits or blocks the binding of the second antigen-binding protein, e.g., via steric hindrance. Competition between antigen-binding proteins may be measured by methods known in the art, e.g., by a real-time, label-free bio-layer interferometry assay.

In the context of the present disclosure, the term “neutralizing antibody” or “nAb” refers to an antibody that binds to a pathogen (e.g., a virus) and interferes with its ability to infect a cell. Non-limiting examples of neutralizing antibodies include antibodies that bind to a viral particle and inhibit successful transduction, e.g., one or more steps selected from binding, entry, trafficking to the nucleus, and transcription of the viral genome. Some neutralizing antibodies may block a virus at the post-entry step.

The term “immune response” refers to a response of a cell of the immune system (e.g., a B-cell, T-cell, macrophage or polymorphonucleocyte) to a stimulus such as an immunogen, e.g., antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host.

The term “T cell” is used herein in its broadest sense to refer to all types of immune cells expressing CD3, including T-helper cells (CD4+ cells), cytotoxic T-cells (CD8+ cells), T-regulatory cells (Treg), and natural killer (NK)-T cells.

The terms “substantial identity” and “substantially identical,” as used with reference to a nucleic acid or fragment thereof, indicates that, when optimally aligned with appropriate nucleotide insertions or deletions with another nucleic acid (or its complementary strand), there is nucleotide sequence identity in at least about 90%, e.g., at least 91%, 92%, 93%, 94%, 95%, 96%, 97%, 98% or 99%, of the nucleotide bases, as measured by any well-known algorithm of sequence identity, such as FASTA, BLAST or GAP, as discussed below. A nucleic acid molecule having substantial identity to a reference nucleic acid molecule may, in certain instances, encode a polypeptide having the same or substantially similar amino acid sequence as the polypeptide encoded by the reference nucleic acid molecule.

As applied to polypeptides, the terms “substantial identity” and ““substantially identical” mean that two peptide sequences, when optimally aligned, share at least about 90% sequence identity, e.g., at least 90%, 91%, 92%, 93%, 94%, 95%, 96%, 97%, 98% or 99% sequence identity. In some embodiments, residue positions that are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein.

Sequence similarity for polypeptides is typically measured using sequence analysis software. Protein analysis software matches similar sequences using measures of similarity assigned to various substitutions, deletions, and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild-type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson, 2000 supra). Another preferred algorithm when comparing a sequence of the disclosure to a database containing a large number of sequences from different organisms is the computer program BLAST, especially BLASTP or TBLASTN, using default parameters. See, e.g., Altschul et al., 1990, J. Mol. Biol. 215: 403-410 and 1997 Nucleic Acids Res. 25:3389-3402.

A “variant” of a polypeptide, such an immunoglobulin, VH, VL, heavy chain, light chain, or CDR comprising an amino acid sequence specifically set forth herein, refers to a polypeptide comprising an amino acid sequence that is at least about 70%-99.9% (e.g., at least 70, 75, 80, 85, 90, 91, 92, 93, 94, 95, 96, 97, 98, 99, 99.5, or 99.9%) identical to the reference polypeptide sequence (e.g., as set forth in the sequence listing below), when the comparison is performed by a BLAST algorithm wherein the parameters of the algorithm are selected to give the largest match between the respective sequences over the entire length of the respective reference sequences. In some embodiments, a variant of a polypeptide includes a polypeptide having the amino acid sequence of a reference polypeptide sequence (e.g., as set forth in the sequence listing below) but for one or more (e.g., 1 to 10, or less than 20, or less than 10) missense mutations (e.g., conservative substitutions), nonsense mutations, deletions, or insertions.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable,” as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

An “individual” or “subject” or “animal” refers to humans, veterinary animals (e.g., cats, dogs, cows, horses, sheep, pigs, etc.) and experimental animal models of diseases (e.g., mice, rats). In a preferred embodiment, the subject is a human.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

“Optional” or “optionally” means that the subsequently described event or circumstance may or may not occur and that the description includes instances in which the event or circumstance occurs and instances in which the event or circumstance does not.

Designation of a range of values includes all integers within or defining the range, and all subranges defined by integers within the range. For example, 5-10 nucleotides is understood as 5, 6, 7, 8, 9, or 10 nucleotides, whereas 5-10% is understood to contain 5% and all possible values through 10%.

At least 17 nucleotides of a 20 nucleotide sequence is understood to include 17, 18, 19, or 20 nucleotides of the sequence provided, thereby providing an upper limit even if one is not specifically provided as it would be clearly understood. Similarly, up to 3 nucleotides would be understood to encompass 0, 1, 2, or 3 nucleotides, providing a lower limit even if one is not specifically provided. When “at least,” “up to,” or other similar language modifies a number, it can be understood to modify each number in the series.

As used herein, “no more than” or “less than” is understood as the value adjacent to the phrase and logical lower values or integers, as logical from context, to zero. For example, a duplex region of “no more than 2 nucleotide base pairs” has a 2, 1, or 0 nucleotide base pairs. When “no more than” or “less than” is present before a series of numbers or a range, it is understood that each of the numbers in the series or range is modified.

As used herein, it is understood that when the maximum amount of a value is represented by 100% (e.g., 100% inhibition) that the value is limited by the method of detection. For example, 100% inhibition is understood as inhibition to a level below the level of detection of the assay.

Unless otherwise apparent from the context, the term “about” encompasses values±5% of a stated value. In certain embodiments, the term “about” is understood to encompass tolerated variation or error within the art, e.g., 2 standard deviations from the mean, or the sensitivity of the method used to take a measurement, or a percent of a value as tolerated in the art, e.g., with age. When “about” is present before the first value of a series, it can be understood to modify each value in the series.

The term “and/or” refers to and encompasses any and all possible combinations of one or more of the associated listed items, as well as the lack of combinations when interpreted in the alternative (“or”).

The term “or” refers to any one member of a particular list and also includes any combination of members of that list.

The singular forms of the articles “a,” “an,” and “the” include plural references unless the context clearly dictates otherwise. For example, the term “a protein” or “at least one protein” can include a plurality of proteins, including mixtures thereof.

Statistically significant means p≤0.05.

In the event of a conflict between a sequence in the application and an indicated accession number or position in an accession number, the sequence in the application predominates.

### DETAILED DESCRIPTION

### I. Overview

Provided herein are methods of inserting a nucleic acid encoding a polypeptide of interest into a target genomic locus in a cell or a population of cells in a subject, methods of expressing a polypeptide of interest from a target genomic locus in a cell or a population of cells in a subject, methods of treating an enzyme deficiency (e.g., FIX deficiency or GAA deficiency) in a subject in need thereof, and methods of preventing or reducing the onset of a sign or symptom of an enzyme deficiency in a subject in need thereof.

Gene transfer technologies generally rely on one or more protein components for the encapsulation, transport, and/or transmission of genetic material. Frequently, these components contain antigenic or immunogenic regions that upon transfer into a living organism can be recognized by the host immune system, leading to immune responses that can impact the initial or long-term effectiveness of gene transfer.

For adeno-associated virus (AAV)-based vectors, which consist of a single-stranded DNA genome packaged in a protein capsid, presence of pre-existing antibodies against the capsid can lead to significantly reduced efficiency of transduction. These antibody responses develop following exposure to either wild type or recombinant AAVs, are usually neutralizing at relatively low titers, and can persist for at least a decade (>10 years), especially following exposure to the high doses required for existing AAV gene therapy products.

Thus, the development and persistence of high-titer neutralizing antibody responses following AAV exposure means that most systemic AAV-based gene therapies are expected to be a once per lifetime treatment, regardless of treatment outcome. This once-per-lifetime treatment paradigm poses several challenges in the clinic. Strategies to mitigate antibody responses to AAV therefore have the potential to greatly benefit patients due to both improved effectiveness and durability of treatment, as well as expanded access to existing and future AAV gene therapies.

Other broad-spectrum immunosuppression methodologies, including broad spectrum immunosuppression (e.g., calcineurin inhibitors [tacrolimus, cyclosporine], rapamycin, MMF, corticosteroids, methotrexate, proteasome inhibitors, costimulation blockade [CTLA4-Ig], Src kinase inhibitors, Btk inhibitors), B cell depletion (rituximab), IgG degrading enzymes (IdeS), IgG half-life reducers (FcRn blockers), or combinations thereof, have not been shown to be effective at enabling AAV vector re-administration at levels equivalent to naïve individuals.

In one aspect, the present disclosure provides a distinct B cell immunosuppression approach that enables AAV vector re-transduction at levels equal to seronegative animals, by depleting pre-existing nAbs (e.g., via combined plasma cell and immunoglobulin depletion). Long-lived plasma cells (LLPC) mediate constitutive antibody production to most antigens and are the likely reservoir of persistent anti-AAV antibody immunity. The present disclosure was made in part based on the discovery that pre-existing AAV nAbs could be directly eliminated in vivo by LLPC depletion with linvoseltamab, a fully-human T cell-bridging bispecific antibody targeting B cell maturation antigen and CD3 (anti-BCMA×CD3 bispecific antibody), either alone or in combination with B cell depletion (to eliminate potential non-LLPC sources of anti-AAV nAbs) and/or FcRn blockade (to accelerate serum IgG clearance).

The methods described herein use plasma cell depleting agents or combinations comprising plasma cell depleting agents to mitigate immune response and facilitate redosing of nucleic acid constructs encoding a polypeptide of interest and nuclease agents targeting a target genomic locus. Optionally, the plasma cell depleting agents (e.g., BCMA×CD3 antigen-binding molecules) are used in combination with other immunosuppression methodologies, such as immunoglobulin depleting agents (e.g., FcRn blockers or IgG degrading enzymes), B cell depleting agents, plasmapheresis, therapeutic plasma exchange, immunoadsorption, broad spectrum immunosuppression, or combinations thereof. In one example, plasma cell depleting agents (e.g., BCMA×CD3 bispecific antigen-binding molecules) are used in combination with immunoglobulin depleting agents (e.g., IgG half-life reducers, such as FcRn blockers). In another example, plasma cell depleting agents (e.g., BCMA×CD3 bispecific antigen-binding molecules) are used in combination with B cell depleting agents (e.g., CD20×CD3 antigen-binding molecules). In another example, BCMA×CD3 bispecific antigen-binding molecules are used in combination with immunoglobulin depleting agents (e.g., FcRn blockers) and B cell depleting agents (e.g., CD20×CD3 antigen-binding molecules). This allows re-dosing of any AAV gene therapy product. For example, for CRISPR-mediated gene insertion platforms consisting of an AAV and LNP, the AAV and/or LNP can be re-dosed multiple times when plasma cell depleting agents or combinations comprising plasma cell depleting agents is co-administered.

Using plasma cell depleting agents or combinations comprising plasma cell depleting agents to mitigate an anti-AAV antibody response can allow for repeated dosing of an identical gene insertion therapeutic cargo. This allows targeted cells in a subject to produce a polypeptide of interest in a step-wise, increasing fashion due to increased gene insertion in additional targeted cells following repeated dosing until a desired level of expression and/or activity of the polypeptide of interest is achieved in a subject without overshooting. This can be particularly advantageous in situations in which overshooting (i.e., achieving higher than desired levels of expression and/or activity of the polypeptide of interest) would result in undesired side effects (e.g., toxicity). Likewise, using plasma cell depleting agents or combinations comprising plasma cell depleting agents to mitigate an anti-AAV antibody response can allow for gene insertion of an AAV template into two separate genomic locations from two discrete dosings (e.g., two discrete dosings of AAV and LNP). Similarly, using plasma cell depleting agents or combinations comprising plasma cell depleting agents to mitigate an anti-AAV antibody response can allow for gene insertion of two different AAV templates (encoding different polypeptides of interest or the same polypeptide of interest) from two discrete dosings (e.g., two discrete dosings of AAV and LNP).

Also provided are compositions or combinations or kits comprising a plasma cell depleting agent or combination comprising a plasma cell depleting agent in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus. As used herein, the term “in combination with” a plasma cell depleting agent means that additional component(s) may be administered prior to, concurrent with, or after the administration of the plasma cell depleting agent. The different components of the combination can be formulated into a single composition, e.g., for simultaneous delivery, or formulated separately into two or more compositions (e.g., a kit including each component, for example, wherein the further agent is in a separate formulation).

In another aspect, the present disclosure provides use of B cell depleting agents such as anti-CD20×CD3 bispecific antibodies or functional fragments thereof to mitigate immune response and facilitate redosing of nucleic acid constructs encoding a polypeptide of interest and nuclease agents targeting a target genomic locus. The B cell depleting agents (e.g., anti-CD20×CD3 bispecific antibodies or functional fragments thereof) are able to suppress host B cell responses to new antigens. In AAV gene therapies, seronegative/naive patients are dosed with AAV and develop antibody responses to the AAV capsid antigen. This antibody response prevents future re-dosing of AAV because the antibodies are neutralizing, and the antibody response is sustained for 10+ years. When AAV is co-administered with B cell depleting agents (e.g., anti-CD20×CD3 bispecific antibodies or functional fragments thereof), the B cell response is suppressed and anti-AAV IgM and IgG responses are significantly suppressed. This allows re-dosing of any AAV gene therapy product. For example, for CRISPR-mediated gene insertion platforms consisting of an AAV and LNP, the AAV and/or LNP can be re-dosed multiple times when B cell depleting agents (e.g., anti-CD20×CD3 bispecific antibodies or functional fragments thereof) are co-administered. The B cell depleting agents (e.g., anti-CD20×CD3 bispecific antibodies or functional fragments thereof) prevent antibody formation against the AAV. The B cell depleting agents (e.g., anti-CD20×CD3 bispecific antibodies or functional fragments thereof) can also prevent antibody formation against certain LNP components (e.g., anti-PEG IgG) or Cas proteins, which can improve efficacy of LNP redosing. In such contexts in which the patient is seronegative/naïve with respect AAV immunity or another immunogen to be administered, the B cell depleting agents (e.g., anti-CD20×CD3 bispecific antibodies or functional fragments thereof) can be used in methods in which a plasma cell depleting agent is not administered. Likewise, in such contexts in which the patient is seronegative/naïve with respect AAV immunity or another immunogen to be administered, the B cell depleting agents (e.g., anti-CD20×CD3 bispecific antibodies or functional fragments thereof) can be used in methods in which an immunoglobulin depleting agent is not administered.

Using B cell depleting agents (e.g., anti-CD20×CD3 bispecific antibodies or functional fragments thereof) to mitigate an anti-AAV antibody response can allow for repeated dosing of an identical gene insertion therapeutic cargo. This allows targeted cells in a subject to produce a polypeptide of interest in a step-wise, increasing fashion due to increased gene insertion in additional targeted cells following repeated dosing until a desired level of expression and/or activity of the polypeptide of interest is achieved in a subject without overshooting. This can be particularly advantageous in situations in which overshooting (i.e., achieving higher than desired levels of expression and/or activity of the polypeptide of interest) would result in undesired side effects (e.g., toxicity). Likewise, using B cell depleting agents (e.g., anti-CD20×CD3 bispecific antibodies or functional fragments thereof) to mitigate an anti-AAV antibody response can allow for gene insertion of an AAV template into two separate genomic locations from two discrete dosings (e.g., two discrete dosings of AAV and LNP). Similarly, using B cell depleting agents (e.g., anti-CD20×CD3 bispecific antibodies or functional fragments thereof) to mitigate an anti-AAV antibody response can allow for gene insertion of two different AAV templates (encoding different polypeptides of interest or the same polypeptide of interest) from two discrete dosings (e.g., two discrete dosings of AAV and LNP).

Other broad-spectrum immunosuppression methodologies have not been shown to be effective at enabling AAV vector re-administration at levels equivalent to naïve individuals. The B cell depleting agents (e.g., anti-CD20×CD3 bispecific antibodies or functional fragments thereof) disclosed herein, can achieve levels of re-transduction similar to naïve animals.

Also provided are compositions or combinations or kits comprising a B cell depleting agent (e.g., anti-CD20×CD3 bispecific antibody or functional fragment thereof) in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus. As used herein, the term “in combination with” a B cell depleting agent (e.g., anti-CD20×CD3 bispecific antibody or functional fragment thereof) means that additional component(s) may be administered prior to, concurrent with, or after the administration of the B cell depleting agent (e.g., anti-CD20×CD3 bispecific antibody or functional fragment thereof). The different components of the combination can be formulated into a single composition, e.g., for simultaneous delivery, or formulated separately into two or more compositions (e.g., a kit including each component, for example, wherein the further agent is in a separate formulation).

### II. Plasma Cell Depleting Agents

In some embodiments, the compositions disclosed herein comprise or the methods disclosed herein include administering a therapeutically effective amount of a plasma cell depleting agent to a subject in need thereof. As used herein, a “plasma cell depleting agent” refers to any molecule capable of specifically binding to a surface antigen on plasma cells and killing or depleting the plasma cells.

The plasma cell depleting agents can be administered to a subject in need thereof either alone, or in combination with, a B cell depleting agent and/or an immunoglobulin depleting agent. In various aspects, a plasma cell depleting agent may be combined or administered in combination with a B cell depleting agent, an immunoglobulin depleting agent, plasmapheresis, therapeutic plasma exchange, immunoadsorption, and/or an immunogen (e.g., nucleic acid construct, nuclease agent or CRISPR/Cas system, e.g., in an immunogenic delivery vehicle such as, e.g., AAV) disclosed herein. Suitable combinations comprising a plasma cell depleting agent are described in more detail elsewhere herein. In some embodiments, the plasma cell depleting agent of the present disclosure is capable of depleting plasma cells including, without limitation, long-lived plasma cells (LLPCs). In some embodiments, a plasma cell depleting agent is administered to a subject having a pre-existing immunity against an immunogen (e.g., an immunogenic delivery vehicle such as, e.g., AAV (e.g., AAV comprising a nucleic acid construct described herein)). In some embodiments, a plasma cell depleting agent is administered to a subject having a pre-existing immunity against a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein. In some embodiments, a plasma cell depleting agent is administered to a subject having a pre-existing immunity against an AAV vector comprising a nucleic acid construct described herein.

As used herein, the term “immunogen” refers to any molecule that is capable of eliciting an immune response. Non-limiting examples of immunogens include immunogenic delivery vehicles such as viral vectors also termed herein “viral particles” (e.g., viral vectors derived from adeno-associated viruses (AAV), adenoviruses, retroviruses [e.g., lentiviruses], or oncolytic viruses [e.g., an adenovirus, a rhabdovirus, a herpes virus, a measles virus, a coxsackievirus, a poliovirus, a reovirus, a poxvirus, a parvovirus, Maraba virus, or Newcastle disease virus]) or portions thereof (e.g., capsid proteins), virus-like particles (VLPs), non-viral vectors (e.g., bacteriophages [such as lambda (X) bacteriophage, EMBL bacteriophage; bacterial vectors such as pBs, phagescript, PsiX174, pBluescript SK, pBs KS, pNH8a, pNH16a, pNH18a, pNH46a; pTrc99A, pKK223-3, pKK233-3, pDR540, and pRIT5]; eukaryotic vectors [such as pWLneo, pSV2cat, pOG44, PXR1, pSG, pSVK3, pBPV, pMSG and pSVL]; transposons [such as Sleeping Beauty transposon and PiggyBac transposon]; bacterial vectors, fungal vectors, and protozoal vectors), liposomes, lipid nanoparticles (LNPs), non-lipid nanoparticles, mammalian cells (e.g., allogeneic cells), and other carriers. Non-limiting examples of immunogens also include polypeptide molecules (e.g., proteins [e.g., therapeutic proteins or antibodies or fragments thereof], peptides), polynucleotide molecules (e.g., mRNAs, interfering nucleic acid molecules [RNAi, siRNA, shRNA], miRNAs, antisense oligonucleotides, ribozymes, aptamers, mixmers, or multimers), antigen-binding molecules fused to a payload, as well as naturally occurring or modified bacteria, fungi, protozoa, parasites, helminths, ectoparasites, or other microorganisms (including bacteria, fungi and other microorganisms found in microbiota). In some embodiments, the immunogen is an immunogenic delivery vehicle, a polypeptide, or a polynucleotide. In some embodiments, the immunogen is an immunogenic delivery vehicle (e.g., AAV) or a polypeptide or polynucleotide encoded by a nucleic acid construct or transgene within the immunogenic delivery vehicle. In some embodiments, the immunogen is an immunogenic delivery vehicle. In some embodiments, the immunogenic delivery vehicle is a viral vector. In some embodiments, the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell. In some embodiments, the immunogenic delivery vehicle is a viral vector, a virus-like particle (VLP), a lipid nanoparticle (LNP), a non-lipid nanoparticle, a liposome, a bacterial vector, a fungal vector, or a protozoal vector. Glycans and lipids are further encompassed by the term immunogen as used herein. In some embodiments, an immunogen can be a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein.

In some embodiments, the plasma cell depleting agent can be an antibody, a small molecule compound, a nucleic acid, a polypeptide, or a functional fragment or variant thereof. Non-limiting examples of suitable plasma cell depleting agents include B cell maturation antigen (BCMA) targeting agents (described elsewhere herein), proteasome inhibitors [e.g., bortezomib (Velcade), carfilzomib (Kyprolis), ixazomib (Niniaro)], histone deacetylase inhibitors [e.g., panobinostat (Farydak)], B-cell activating factor (BAFF; also referred to as BLyS, TALL-1, or CD257) inhibitors (e.g., anti-BAFF antibodies such as belimumab, tabalumab, AMG570; or anti-BAFF receptor antibodies such as ianalumab), proliferation-inducing ligand (APRIL; also referred to as TNFSF13 or CD256) inhibitors (e.g., anti-APRIL antibodies such as BION-1301 or VIS624), G protein-coupled receptor, class C, group 5, member D (GPRC5D) inhibitors (e.g., anti-GPRC5D antibodies, anti-GPRC5D×CD3 bispecific antibodies such as talquetamab), Fc receptor homolog 5 (FcRH5; also referred to as FcRL5, IRTA2, or CD307) inhibitors (e.g., anti-FcRH5 antibodies, anti-FcRH5 xCD3 bispecific antibodies such as Cevostamab), and cluster of differentiation 38 (CD38; also referred to as CADPR 1 or ADPRC1) inhibitors (e.g., anti-CD38 antibodies).

In some embodiments, the plasma cell depleting agents used in the compositions and methods disclosed herein are BCMA targeting agents. As used herein, the term “BCMA targeting agent” refers to any molecule capable of binding specifically to BCMA that is expressed on the surface of a cell, e.g., a cell in a subject, thus targeting the cell for destruction. BCMA is expressed exclusively in B-cell lineage cells, particularly in the interfollicular region of the germinal center as well as on plasmablasts and differentiated plasma cells. BCMA is selectively induced during plasma cell differentiation and is required for optimal survival of long-lived plasma cells (LLPCs) in the bone marrow. Thus, a BCMA targeting agent binds to BCMA expressed on a plasma cell surface and mediates killing or depletion of cells that express BCMA (plasma cell depletion). In some embodiments, a BCMA targeting agent comprises a binding moiety that binds to plasma cell-surface-expressed BCMA (an antigen-binding moiety or antigen-binding fragment thereof) and a moiety that facilitates killing of said plasma cell. In some embodiments, the plasma cell-surface-expressed BCMA-binding moiety is an antibody or antigen-binding fragment thereof that binds specifically to BCMA. Such a BCMA-binding moiety can be linked (e.g., covalently bound) to a moiety that facilitates killing or destruction of the targeted plasma cell. The moiety that facilitates targeted killing of the bound plasma cell may be a molecule that directly kills the targeted cell (e.g., a cytotoxic agent) or may be a protein or fragment thereof that mediates killing of the targeted cell, e.g., by an immune cell, e.g., a T-cell. In the context of the present disclosure, the term “BCMA targeting agent” includes, but is not limited to, anti-BCMA antibodies that are conjugated to a therapeutic agent such as a cytotoxic drug (“BCMA ADC” or “anti-BCMA ADC,” e.g., Belantamab Mafodotin/GSK2857916, MEDI2228, HDP-101), chimeric antigenic receptors (CARs) that bind specifically to BCMA, (“BCMA CAR” or “anti-BCMA CAR”) and anti-BCMA×CD3 bispecific antibodies (e.g., linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B).

In some embodiments, the BCMA targeting agent used in the context of the disclosed methods is an antibody-drug conjugate (ADC) comprising an anti-BCMA antibody and a cytotoxic drug. In some embodiments, the anti-BCMA antibody or antigen-binding fragment thereof and the cytotoxic agent are covalently attached via a linker. In general terms, the ADCs comprise: A-[L-P]_{y}, in which A is an antigen-binding molecule, e.g., an anti-BCMA antibody, or a fragment thereof, L is a linker, P is the payload or therapeutic moiety (e.g., cytotoxic agent), and y is an integer from 1 to 30. Examples of suitable cytotoxic agents and chemotherapeutic agents for forming ADCs are known in the art. Non-limiting examples of suitable cytotoxic agents that can be conjugated to anti-BCMA antibodies for use in the disclosed methods are auristatin such as monomethyl auristatin E (MMAE) or monomethyl auristatin F (MMAF), a tubulysin such as TUB-OH or TUB-OMOM, a tomaymycin derivative, a dolastatin derivative, or a maytansinoid such as DM1 or DM4. In some exemplary embodiments, an anti-BCMA ADC used in the present methods comprises the HCVR, LCVR and/or CDR amino acid sequences of any of the anti-BCMA antigen-binding molecules disclosed herein.

Other anti-BCMA ADCs that can be used in the context of the methods of the present disclosure include, e.g., the ADCs referred to and known in the art as Belantamab Mafodotin (GSK2857916), AMG224, HDP-101, MED12228, and TBL-CLN1, or any of the anti-BCMA ADCs set forth, e.g., in International Patent Publications WO2011/108008, WO2014/089335, WO2017/093942, WO2017/143069, or WO2019/025983. The portions of the publications cited herein that identify anti-BCMA ADCs are hereby incorporated by reference.

In some embodiments, the BCMA targeting agent used in the context of the disclosed methods is a chimeric antigen receptor (CAR) that binds specifically to BCMA (“BCMA CAR”). Generally, a “chimeric antigen receptor” (CAR) exhibits a specific anti-target cellular immune activity and comprises a binding domain against a component present on the target cell, for example an antibody-based specificity for a desired antigen (e.g., BCMA on plasma cell), and a T cell receptor-activating intracellular domain. CARs typically comprise an extracellular single chain antibody-binding domain (scFv) fused to the intracellular signaling domain of the T cell antigen receptor complex zeta chain, and have the ability, when expressed in T cells, to redirect antigen recognition based on the monoclonal antibody's specificity. In certain embodiments, the BCMA CAR or antigen-binding fragment thereof comprises a HCVR, LCVR, and/or CDRs comprising the amino acid sequences of any of the antibodies set forth in US Patent Publication No. US 2020/0023010, which is hereby incorporated by reference in its entirety. In some exemplary embodiments, an anti-BCMA CAR used in the present methods comprises the HCVR, LCVR and/or CDR amino acid sequences of any of the anti-BCMA antigen-binding molecules disclosed herein.

Other anti-BCMA CARs that can be used in the context of the methods of the present disclosure include, e.g., the CARs referred to and known in the art as bb2121, LCAR-B38M, and 4C8A, or any of the anti-BCMA CARs set forth, e.g., in WO 2015/052538, WO 2015/052536, WO 2016/094304, WO 2016/166630, WO 2016/151315, WO 2016/130598, WO 2017/183418, WO 2017/173256, WO 2017211900, WO 2017/130223, WO 2018/229492, WO 2018/085690, WO 2018/151836, WO 2018/028647, WO 2019/006072. The portions of the publications cited herein that identify anti-BCMA CARs are hereby incorporated by reference.

In some exemplary embodiments, the BCMA targeting agent used in the disclosed methods is a multispecific (e.g., bispecific) antibody, or a functional fragment thereof, that specifically binds B cell maturation antigen (BCMA) and CD3 (e.g., an anti-BCMA×CD3 bispecific antibody). The anti-BCMA×CD3 multispecific (e.g., bispecific) antibodies are useful for specific targeting and T-cell-mediated killing of cells that express BCMA. The terms “antibody,” “antigen-binding fragment,” “human antibody,” “recombinant antibody,” and other related terminology are defined above. In the context of anti-BCMA×CD3 antibodies and antigen-binding fragments thereof, the present disclosure includes the use of bispecific antibodies wherein one arm of an immunoglobulin is specific for BCMA or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. See, e.g., Kazane et al., J. Am. Chem. Soc., 2013, 135(1):340-46.

An anti-BCMA×CD3 bispecific antibody, or functional fragment thereof, may comprise any of various anti-BCMA×CD3 bispecific antibodies, or functional fragments thereof, disclosed herein, or any other such anti-BCMA×CD3 bispecific antibodies, or functional fragments thereof, known to persons of ordinary skill in the art (e.g., linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B). In a specific embodiment, the anti-BCMA×CD3 bispecific antibody is REGN5458. In another specific embodiment, the anti-BCMA×CD3 bispecific antibody is REGN5459.

### A. BCMA×CD3 Antigen-Binding Molecules

In some embodiments, the present disclosure provides antigen-binding molecules including multispecific (e.g., bispecific) antibodies that specifically bind B cell maturation antigen (BCMA) and CD3 (e.g., an anti-BCMA×CD3 bispecific antibody). In some embodiments, the antigen-binding molecule is a multispecific (e.g., bispecific) antibody. Multispecific antibodies may be specific for different epitopes of one target polypeptide or may contain antigen-binding domains specific for more than one target polypeptide. See, e.g., Tutt et al., 1991, J. Immunol. 147:60-69; Kufer et al., 2004, Trends Biotechnol. 22:238-244. In some embodiments, the multispecific antibodies of the present disclosure can be linked to or co-expressed with another functional molecule, e.g., another peptide or protein. For example, an antibody or fragment thereof can be functionally linked (e.g., by chemical coupling, genetic fusion, noncovalent association or otherwise) to one or more other molecular entities, such as another antibody or antibody fragment to produce a bispecific or a multispecific antibody with a second binding specificity. In some embodiments, the multispecific antibody contains an antigen-binding domain that is specific for BCMA and an antigen-binding domain that is specific for CD3.

The term “CD3,” as used herein, refers to an antigen which is expressed on T cells as part of the multimolecular T cell receptor (TCR) and which consists of a homodimer or heterodimer formed from the dimeric association of two of four receptor chains: CD3-epsilon, CD3-delta, CD3-zeta, and CD3-gamma (e.g., gamma/epsilon, delta/epsilon, and zeta/zeta). CD3 is required for T cell activation.

As used herein, “an antibody that binds CD3” or an “anti-CD3 antibody” includes antibodies and antigen-binding fragments thereof that specifically recognize a single CD3 subunit (e.g., epsilon, delta, gamma or zeta), as well as antibodies and antigen-binding fragments thereof that specifically recognize a dimeric complex of two CD3 subunits (e.g., gamma/epsilon, delta/epsilon, and zeta/zeta CD3 dimers). Antibodies against CD3 have been shown to cluster CD3 on T cells, thereby causing T cell activation in a manner similar to the engagement of the TCR by peptide-loaded major histocompatibility complex (MHC) molecules. Thus, bispecific antigen-binding molecules that are capable of binding both CD3 and another antigen (e.g., CD20 or BCMA) would be useful in settings in which specific targeting and T cell-mediated killing of cells that express the non-CD3 antigen (e.g., CD20 or BCMA) is desired.

The antibodies and antigen-binding fragments of the present invention may bind soluble CD3 and/or cell surface-expressed CD3. Soluble CD3 includes natural CD3 proteins as well as recombinant CD3 protein variants such as, e.g., monomeric and dimeric CD3 constructs, that lack a transmembrane domain or are otherwise unassociated with a cell membrane.

As used herein, the expression “cell surface-expressed CD3” means one or more CD3 protein(s) that is/are expressed on the surface of a cell in vitro or in vivo, such that at least a portion of a CD3 protein is exposed to the extracellular side of the cell membrane and is accessible to an antigen-binding portion of an antibody. “Cell surface-expressed CD3” includes CD3 proteins contained within the context of a functional T cell receptor in the membrane of a cell. The expression “cell surface-expressed CD3” includes CD3 protein expressed as part of a homodimer or heterodimer on the surface of a cell (e.g., gamma/epsilon, delta/epsilon, and zeta/zeta CD3 dimers). The expression “cell surface-expressed CD3” also includes a CD3 chain (e.g., CD3-epsilon, CD3-delta or CD3-gamma) that is expressed by itself, without other CD3 chain types, on the surface of a cell. A “cell surface-expressed CD3” can comprise or consist of a CD3 protein expressed on the surface of a cell which normally expresses CD3 protein. Alternatively, “cell surface-expressed CD3” can comprise or consist of CD3 protein expressed on the surface of a cell that normally does not express human CD3 on its surface but has been artificially engineered to express CD3 on its surface.

As used herein, the expression “anti-CD3 antibody” includes both monovalent antibodies with a single specificity, as well as bispecific antibodies comprising one arm that binds CD3 and another arm that binds a different antigen, wherein the anti-CD3 arm comprises any of the HCVR/LCVR or CDR sequences, or functional fragments thereof, as set forth in Table 1 or Table 2 herein. Examples of anti-CD3 bispecific antibodies are described elsewhere herein. Exemplary anti-CD3 antibodies are also described in PCT International Application No. PCT/US2013/060511, which is herein incorporated by reference in its entirety.

The present disclosure includes bispecific antibodies and functional fragments thereof that bind human CD3 with high affinity. The present disclosure also includes bispecific antibodies and functional fragments thereof that bind human CD3 with medium or low affinity, depending on the therapeutic context and particular targeting properties that are desired. For example, in the context of a bispecific antigen-binding molecule, wherein one arm binds CD3 and a second arm binds another antigen (e.g., CD20 or BCMA), it may be desirable for the second arm to bind the non-CD3 (e.g., CD20 or BCMA) antigen with high affinity while the anti-CD3 arm binds CD3 with only moderate or low affinity. In this manner, preferential targeting of the antigen-binding molecule to cells expressing the non-CD3 (e.g., CD20 or BCMA) antigen may be achieved while avoiding general/untargeted CD3 binding and the consequent adverse side effects associated therewith.

In certain embodiments, the anti-CD3 antibodies induce T cell proliferation with an EC₅₀ value of less than about 0.33 pM, as measured by an in vitro T cell proliferation assay (e.g., assessing the proliferation of Jurkat cells or PBMCs in the presence of anti-CD3 antibodies). In certain embodiments, the anti-CD3 antibodies induce T cell proliferation (e.g., Jurkat cell proliferation and/or PBMC proliferation) with an EC₅₀ value of less than about 0.32 pM, less than about 0.31 pM, less than about 0.30 pM, less than about 0.28 pM, less than about 0.26 pM, less than about 0.24 pM, less than about 0.22 pM, or less than about 0.20 pM, as measured by an in vitro T cell proliferation assay.

In some embodiments, the anti-BCMA×CD3 bispecific antigen-binding molecule comprises a first antigen-binding domain (D1) that binds an epitope of BCMA (e.g., human BCMA), and a second antigen-binding domain (D2) that binds an epitope of CD3 (e.g., human CD3).

In some exemplary embodiments, the anti-BCMA×CD3 bispecific antibody, or antigen-binding fragment thereof, comprises a heavy chain variable region (HCVR), light chain variable region (LCVR), and/or complementarity determining regions (CDRs) comprising the amino acid sequences of any of the anti-BCMA×CD3 antibodies set forth in U.S. Pat. No. 11,384,153 and US 2020/0345843, which are hereby incorporated by reference in their entireties.

In some exemplary embodiments, an anti-BCMA×CD3 bispecific antibody or antigen-binding fragment thereof that can be used in the context of the present disclosure comprising a HCVR, a LCVR, and/or CDRs comprising the amino acid sequences of REGN5458 or REGN5459 as set forth in Table 1 below.

TABLE 1
Amino Acid Sequences of Exemplary Anti-BCMAxCD3 Bispecific
Antibodies.

| | Anti-BCMA | | | | Anti-CD3 | | | | Common | | | |
|---|---|---|---|---|---|---|---|---|---|---|---|---|
| Bispecific | First Antigen-Binding | | | | Second Antigen-Binding | | | | Light Chain Variable | | | |
| antibody | Domain | | | | Domain | | | | Region | | | |
| identifier | HCVR | HCDR1 | HCDR2 | HCDR3 | HCVR | HCDR1 | HCDR2 | HCDR3 | LCVR | LCDR1 | LCDR2 | LCDR3 |
| REGN5458 | 2 | 4 | 6 | 8 | 26 | 28 | 30 | 32 | 18 | 20 | 22 | 24 |
| REGN5459 | 2 | 4 | 6 | 8 | 34 | 36 | 38 | 40 | 18 | 20 | 22 | 24 |

Anti-BCMA HCVR DNA Sequence
SEQ ID NO: 1
GAGGTGCAGCTGGTGGAGTCTGGGGGAGGCTTGGTCCAGCCTGGGGGGTCCCTGAGACTCTCCTGTGCAGCCTCTGG
ATTCACCTTTAGTAACTTTTGGATGACCTGGGTCCGCCAGGCTCCAGGGAAGGGGCTGGAGTGGGTGGCCAACATGA
ACCAAGATGGAAGTGAGAAATACTATGTGGACTCTGTGAAGGGCCGATTCACCATCTCCAGAGACAACGCCAAGAGC
TCACTGTATCTGCAAATGAACAGCCTGAGAGCCGAGGACACGGCTGTGTATTACTGTGCGAGAGATCGGGAATATTG
TATTAGTACCAGCTGCTATGATGACTTTGACTACTGGGGCCAGGGAACCCTGGTCACCGTCTCCTCA
Anti-BCMA HCVR Protein Sequence
SEQ ID NO: 2
EVQLVESGGGLVQPGGSLRLSCAASGFTFSNFWMTWVRQAPGKGLEWVANMNQDGSEKYYVDSVKGRFTISRDNAKS
SLYLQMNSLRAEDTAVYYCARDREYCISTSCYDDFDYWGQGTLVTVSS
Anti-BCMA HCDR1 DNA Sequence
SEQ ID NO: 3
GGATTCACCTTTAGTAACTTTTGG
Anti-BCMA HCDR1 Protein Sequence
SEQ ID NO: 4
GFTFSNFW
Anti-BCMA HCDR2 DNA Sequence
SEQ ID NO: 5
ATGAACCAAGATGGAAGTGAGAAA
Anti-BCMA HCDR2 Protein Sequence
SEQ ID NO: 6
MNQDGSEK
Anti-BCMA HCDR3 DNA Sequence
SEQ ID NO: 7
GCGAGAGATCGGGAATATTGTATTAGTACCAGCTGCTATGATGACTTTGACTAC
Anti-BCMA HCDR3 Protein Sequence
SEQ ID NO: 8
ARDREYCISTSCYDDFDY
Anti-BCMA LCVR DNA Sequence
SEQ ID NO: 9
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCATAGTGGGGTCCCATCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
Anti-BCMA LCVR Protein Sequence
SEQ ID NO: 10
DIQMTQSPSSLSASVGDRVTITCRASQSISSYLNWYQQKPGKAPKLLIYAASSLHSGVPSRFSGSGSGTDFTLTISS
LQPEDFATYYCQQSYSTPPITFGQGTRLEIK
Anti-BCMA LCDR1 DNA Sequence
SEQ ID NO: 11
CAGAGCATTAGCAGCTAT
Anti-BCMA LCDR1 Protein Sequence
SEQ ID NO: 12
QSISSY
Anti-BCMA LCDR2 DNA Sequence
SEQ ID NO: 13
GCTGCATCC
Anti-BCMA LCDR2 Protein Sequence
SEQ ID NO: 14
AAS
Anti-BCMA LCDR3 DNA Sequence
SEQ ID NO: 15
CAACAGAGTTACAGTACCCCTCCGATCACC
Anti-BCMA LCDR3 Protein Sequence
SEQ ID NO: 16
QQSYSTPPIT
Common LCVR DNA Sequence
SEQ ID NO: 17
GACATCCAGATGACCCAGTCTCCATCCTCCCTGTCTGCATCTGTAGGAGACAGAGTCACCATCACTTGCCGGGCAAG
TCAGAGCATTAGCAGCTATTTAAATTGGTATCAGCAGAAACCAGGGAAAGCCCCTAAGCTCCTGATCTATGCTGCAT
CCAGTTTGCAAAGTGGGGTCCCGTCAAGGTTCAGTGGCAGTGGATCTGGGACAGATTTCACTCTCACCATCAGCAGT
CTGCAACCTGAAGATTTTGCAACTTACTACTGTCAACAGAGTTACAGTACCCCTCCGATCACCTTCGGCCAAGGGAC
ACGACTGGAGATTAAA
Common LCVR Protein Sequence
SEQ ID NO: 18
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIK
Common LCDR1 DNA Sequence
SEQ ID NO: 19
CAGAGCATTAGCAGCTAT
Common LCDR1 Protein Sequence
SEQ ID NO: 20
QSISSY
Common LCDR2 DNA Sequence
SEQ ID NO: 21
GCTGCATCC
Common LCDR2 Protein sequence
SEQ ID NO: 22
AAS
Common LCDR3 DNA sequence
SEQ ID NO: 23
CAACAGAGTTACAGTACCCCTCCGATCACC
Common LCDR3 Protein Sequence
SEQ ID NO: 24
QQSYSTPPIT
Anti-CD3 HCVR DNA Sequence-REGN5458
SEQ ID NO: 25
GAAGTACAGCTTGTAGAATCCGGCGGAGGACTGGTACAACCTGGAAGAAGTCTTAGACTGAGTTGCGCAGCTAGTGG
GTTTACATTCGACGATTACAGCATGCATTGGGTGAGGCAAGCTCCTGGTAAAGGATTGGAATGGGTTAGCGGGATAT
CATGGAACTCAGGAAGCAAGGGATACGCCGACAGCGTGAAAGGCCGATTTACAATATCTAGGGACAACGCAAAAAAC
TCTCTCTACCTTCAAATGAACTCTCTTAGGGCAGAAGACACAGCATTGTATTATTGCGCAAAATACGGCAGTGGTTA
TGGCAAGTTTTATCATTATGGACTGGACGTGTGGGGACAAGGGACAACAGTGACAGTGAGTAGC
Anti-CD3 HCVR Protein Sequence-REGN5458
SEQ ID NO: 26
EVQLVESGGGLVQPGRSLRLSCAASGFTFDDYSMHWVRQAPGKGLEWVSGISWNSGSKGYADSVKGRFTISRDNAKN
SLYLQMNSLRAEDTALYYCAKYGSGYGKFYHYGLDVWGQGTTVTVSS
Anti-CD3 HCDR1 DNA Sequence-REGN5458
SEQ ID NO: 27
GGGTTTACATTCGACGATTACAGC
Anti-CD3 HCDR1 Protein Sequence-REGN5458
SEQ ID NO: 28
GFTFDDYS
Anti-CD3 HCDR2 DNA Sequence-REGN5458
SEQ ID NO: 29
ATATCATGGAACTCAGGAAGCAAG
Anti-CD3 HCDR2 Protein Sequence-REGN5458
SEQ ID NO: 30
ISWNSGSK
Anti-CD3 HCDR3 DNA Sequence-REGN5458
SEQ ID NO: 31
GCAAAATACGGCAGTGGTTATGGCAAGTTTTATCATTATGGACTGGACGTG
Anti-CD3 HCDR3 Protein Sequence-REGN5458
SEQ ID NO: 32
AKYGSGYGKFYHYGLDV
Anti-CD3 HCVR DNA Sequence-REGN5459
SEQ ID NO: 33
GAAGTACAGCTTGTAGAATCCGGCGGAGGACTGGTACAACCTGGAAGAAGTCTTAGACTGAGTTGCGCAGCTAGTGG
GTTTACATTCGACGATTACAGCATGCATTGGGTGAGGCAAGCTCCTGGTAAAGGATTGGAATGGGTTAGCGGGATAT
CATGGAACTCAGGAAGCATCGGATACGCCGACAGCGTGAAAGGCCGATTTACAATATCTAGGGACAACGCAAAAAAC
TCTCTCTACCTTCAAATGAACTCTCTTAGGGCAGAAGACACAGCATTGTATTATTGCGCAAAATACGGCAGTGGTTA
TGGCAAGTTTTATTATTATGGAATGGACGTGTGGGGACAAGGGACAACAGTGACAGTGAGTAGC
Anti-CD3 HCVR Protein Sequence-REGN5459
SEQ ID NO: 34
EVQLVESGGGLVQPGRSLRLSCAASGFTFDDYSMHWVRQAPGKGLEWVSGISWNSGSIGYADSVKGRFTISRDNAKN
SLYLQMNSLRAEDTALYYCAKYGSGYGKFYYYGMDVWGQGTTVTVSS
Anti-CD3 HCDR1 DNA Sequence-REGN5459
SEQ ID NO: 35
GGGTTTACATTCGACGATTACAGC
Anti-CD3 HCDR1 Protein Sequence-REGN5459
SEQ ID NO: 36
GFTFDDYS
Anti-CD3 HCDR2 DNA Sequence-REGN5459
SEQ ID NO: 37
ATATCATGGAACTCAGGAAGCATC
Anti-CD3 HCDR2 Protein Sequence-REGN5459
SEQ ID NO: 38
ISWNSGSI
Anti-CD3 HCDR3 DNA Sequence-REGN5459
SEQ ID NO: 39
GCAAAATACGGCAGTGGTTATGGCAAGTTTTATTATTATGGAATGGACGTG
Anti-CD3 HCDR3 Protein Sequence-REGN5459
SEQ ID NO: 40
AKYGSGYGKFYYYGMDV
Anti-BCMA Heavy Chain Protein Sequence (IgG4 Heavy Chain Constant Region)
SEQ ID NO: 41
EVQLVESGGGLVQPGGSLRLSCAASGFTFSNFWMTWVRQAPGKGLEWVANMNQDGSEKYYVDSVKGRFTISRDNAKS
SLYLQMNSLRAEDTAVYYCARDREYCISTSCYDDFDYWGQGTLVTVSSASTKGPSVFPLAPCSRSTSESTAALGCLV
KDYFPEPVTVSWNSGALTSGVHTFPAVLQSSGLYSLSSVVTVPSSSLGTKTYTCNVDHKPSNTKVDKRVESKYGPPC
PPCPAPPVAGPSVFLFPPKPKDTLMISRTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPSQEEMTKNQVSLTCLVKGFYPSDIAVEW
ESNGQPENNYKTTPPVLDSDGSFFLYSRLTVDKSRWQEGNVFSCSVMHEALHNHYTQKSLSLSLGK
Anti-CD3 Heavy Chain Protein Sequence (IgG4 Heavy Chain Constant Region with
H435R/Y436F)
SEQ ID NO: 42
EVQLVESGGGLVQPGRSLRLSCAASGFTFDDYSMHWVRQAPGKGLEWVSGISWNSGSKGYADSVKGRFTISRDNAKN
SLYLQMNSLRAEDTALYYCAKYGSGYGKFYHYGLDVWGQGTTVTVSSASTKGPSVFPLAPCSRSTSESTAALGCLVK
DYFPEPVTVSWNSGALTSGVHTFPAVLQSSGLYSLSSVVTVPSSSLGTKTYTCNVDHKPSNTKVDKRVESKYGPPCP
PCPAPPVAGPSVFLFPPKPKDTLMISRTPEVTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLPPSQEEMTKNQVSLTCLVKGFYPSDIAVEWE
SNGQPENNYKTTPPVLDSDGSFFLYSRLTVDKSRWQEGNVFSCSVMHEALHNRFTQKSLSLSPGK
Common Anti-BCMA and Anti-CD3 Light Chain Protein Sequence (Kappa Light Chain
Constant Region)
SEQ ID NO: 43
DIQMTQSPSSLSASVGDRVTITCRASQSISSYLNWYQQKPGKAPKLLIYAASSLQSGVPSRFSGSGSGTDFTLTISS
LQPEDFATYYCQQSYSTPPITFGQGTRLEIKRTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC

In some embodiments, the anti-BCMA×CD3 bispecific antibody or antigen-binding fragment thereof that can be used the present disclosure comprises: (a) a first antigen binding domain that binds specifically to BCMA; and (b) a second antigen-binding domain that binds specifically to CD3. In one embodiment, the anti-BCMA antigen-binding domain comprises the heavy chain complementarity determining regions (HCDRs) of a heavy chain variable region (HCVR) comprising the amino acid sequence of SEQ ID NO: 2 and the light chain complementarity determining regions (LCDRs) of a light chain variable region (LCVR) comprising the amino acid sequence of SEQ ID NO: 18. In one embodiment, the first antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 4; the HCDR2 comprises the amino acid sequence of SEQ ID NO: 6; the HCDR3 comprises the amino acid sequence of SEQ ID NO: 8; the LCDR1 comprises the amino acid sequence of SEQ ID NO: 20; the LCDR2 comprises the amino acid sequence of SEQ ID NO: 22; and the LCDR3 comprises the amino acid sequence of SEQ ID NO: 24. In one embodiment, the second antigen-binding domain comprises the heavy chain complementarity determining regions (HCDRs) of a heavy chain variable region (HCVR) comprising the amino acid sequence of SEQ ID NO: 26 or 34 and the light chain complementarity determining regions (LCDRs) of a light chain variable region (LCVR) comprising the amino acid sequence of SEQ ID NO: 18. In one embodiment, the second antigen-binding domain comprises three HCDRs (HCDR1, HCDR2 and HCDR3) and three LCDRs (LCDR1, LCDR2 and LCDR3), wherein the HCDR1 comprises the amino acid sequence of SEQ ID NO: 28 or 36; the HCDR2 comprises the amino acid sequence of SEQ ID NO: 30 or 38; the HCDR3 comprises the amino acid sequence of SEQ ID NO: 32 or 40; the LCDR1 comprises the amino acid sequence of SEQ ID NO: 20; the LCDR2 comprises the amino acid sequence of SEQ ID NO: 22; and the LCDR3 comprises the amino acid sequence of SEQ ID NO: 24.

In one embodiment, the anti-BCMA×CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOS: 4, 6, and 8, and LCDR1, LCDR2, and LCDR3 domains, respectively, comprising the amino acid sequences of SEQ ID NOS: 20, 22, and 24; and (b) a second antigen binding domain that comprises HCDR1, HCDR2, and HCDR3 domains, respectively, comprising the amino acid sequences of SEQ ID NOS: 28, 30, and 32, and LCDR1, LCDR2, and LCDR3 domains, respectively, comprising the amino acid sequences of SEQ ID NOS: 20, 22, and 24. In one embodiment, the anti-BCMA×CD3 bispecific antibody or antigen-binding fragment thereof comprises: (a) a first antigen-binding domain that comprises a HCVR comprising the amino acid sequence of SEQ ID NO: 2 and a LCVR comprising the amino acid sequence of SEQ ID NO: 18; and (b) a second antigen-binding domain that comprises a HCVR comprising the amino acid sequence of SEQ ID NO: 26 and a LCVR comprising the amino acid sequence of SEQ ID NO: 18.

In one embodiment, the anti-BCMA×CD3 bispecific antibody or antigen-binding fragment thereof comprises: (a) a first antigen-binding domain that comprises HCDR1, HCDR2, and HCDR3 domains, respectively, comprising the amino acid sequences of SEQ ID NOS: 4, 6, and 8, and LCDR1, LCDR2, and LCDR3 domains, respectively, comprising the amino acid sequences of SEQ ID NOS: 20, 22, and 24; and (b) a second antigen binding domain that comprises HCDR1, HCDR2, and HCDR3 domains, respectively, comprising the amino acid sequences of SEQ ID NOS: 36, 38, and 40, and LCDR1, LCDR2, and LCDR3 domains, respectively, comprising the amino acid sequences of SEQ ID NOS: 20, 22, and 24. In one embodiment, the anti-BCMA/anti-CD3 bispecific antibody or antigen-binding fragment thereof comprises: (a) a first antigen-binding domain that comprises a HCVR comprising the amino acid sequence of SEQ ID NO: 2 and a LCVR comprising the amino acid sequence of SEQ ID NO:18; and (b) a second antigen-binding domain that comprises a HCVR comprising the amino acid sequence of SEQ ID NO: 34 and a LCVR comprising the amino acid sequence of SEQ ID NO: 18.

Exemplary anti-BCMA×CD3 bispecific antibodies include the fully human bispecific antibodies known as REGN5458 and REGN5459. See, e.g., WO 2020/018820, US 2020/0024356, US 2022/0306758, and U.S. Pat. No. 11,384,153, each of which is herein incorporated by reference. According to certain exemplary embodiments, the methods of the present disclosure comprise the use of REGN5458 or REGN5459, or a bioequivalent thereof. As used herein, the term “bioequivalent” with respect to anti-BCMA×CD3 antibodies refers to antibodies or BCMA×CD3 binding proteins or fragments thereof that are pharmaceutical equivalents or pharmaceutical alternatives having a rate and/or extent of absorption that does not show a significant difference with that of a reference antibody (e.g., REGN5458 or REGN5459) when administered at the same molar dose under similar experimental conditions, either single dose or multiple dose; the term “bioequivalent” also includes antigen-binding proteins that bind to BCMA/CD3 and do not have clinically meaningful differences with the reference antibody (e.g., REGN5458 or REGN5459) with respect to safety, purity and/or potency.

In some embodiments, the anti-BCMA×CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 2 and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 26 and a LCVR having at least 90%, 91%, 92%, 93%, 94%, 95%, 96%, 97%, 98%, or 99% sequence identity to the amino acid sequence of SEQ ID NO: 18. In some embodiments, the anti-BCMA×CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOS: 4, 6, and 8, respectively, and a HCVR having at least 90%, 91%, 92%, 93%, 94%, 95%, 96%, 97%, 98%, or 99% sequence identity to the amino acid sequence of SEQ ID NO: 2, and comprises three LCDRs (LCDR1, LCDR2 and LCDR3) comprising the amino acid sequences of SEQ ID NOS: 20, 22, and 24, respectively, and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises three HCDRs (HCDR1, HCDR2 and HCDR3) comprising the amino acid sequences of SEQ ID NOS: 28, 30, and 32, respectively, and a HCVR having at least 90%, 91%, 92%, 93%, 94%, 95%, 96%, 97%, 98%, or 99% sequence identity to the amino acid sequence of SEQ ID NO: 26, and comprises three LCDRs (LCDR1, LCDR2 and LCDR3) comprising the amino acid sequences of SEQ ID NOS: 20, 22, and 24, respectively, and a LCVR having at least 90%, 91%, 92%, 93%, 94%, 95%, 96%, 97%, 98%, or 99% sequence identity to the amino acid sequence of SEQ ID NO: 18.

In some embodiments, the anti-BCMA×CD3 bispecific antibody or antigen-binding fragment thereof comprises: (a) a first antigen-binding domain that comprises a HCVR having at least 90%, 91%, 92%, 93%, 94%, 95%, 96%, 97%, 98%, or 99% sequence identity to the amino acid sequence of SEQ ID NO: 2 and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises a HCVR having at least 90%, 91%, 92%, 93%, 94%, 95%, 96%, 97%, 98%, or 99% sequence identity to the amino acid sequence of SEQ ID NO: 34 and a LCVR having at least 90%, 91%, 92%, 93%, 94%, 95%, 96%, 97%, 98%, or 99% sequence identity to the amino acid sequence of SEQ ID NO: 18. In some embodiments, the anti-BCMA×CD3 bispecific antibody or antigen-binding fragment thereof comprises: (a) a first antigen-binding domain that comprises three HCDRs (HCDR1, HCDR2 and HCDR3) comprising the amino acid sequences of SEQ ID NOS: 4, 6, and 8, respectively, and a HCVR having at least 90%, 91%, 92%, 93%, 94%, 95%, 96%, 97%, 98%, or 99% sequence identity to the amino acid sequence of SEQ ID NO: 2, and comprises three LCDRs (LCDR1, LCDR2 and LCDR3) comprising the amino acid sequences of SEQ ID NOS: 20, 22, and 24, respectively, and a LCVR having at least 90%, 91%, 92%, 93%, 94%, 95%, 96%, 97%, 98%, or 99% sequence identity to the amino acid sequence of SEQ ID NO: 18; and (b) a second antigen-binding domain that comprises three HCDRs (HCDR1, HCDR2 and HCDR3) comprising the amino acid sequences of SEQ ID NOS: 36, 38, and 40, respectively, and a HCVR having at least 90%, 91%, 92%, 93%, 94%, 95%, 96%, 97%, 98%, or 99% sequence identity to the amino acid sequence of SEQ ID NO: 34, and comprises three LCDRs (LCDR1, LCDR2 and LCDR3) comprising the amino acid sequences of SEQ ID NOS: 20, 22, and 24, respectively, and a LCVR having at least 90%, 91%, 92%, 93%, 94%, 95%, 96%, 97%, 98%, or 99% sequence identity to the amino acid sequence of SEQ ID NO: 18.

The present disclosure also includes variants of the anti-BCMA×CD3 antibodies described herein comprising any of the HCVR, LCVR and/or CDR amino acid sequences disclosed herein with one or more conservative amino acid substitutions. For example, the present disclosure includes use of anti-BCMA×CD3 antibodies having HCVR, LCVR and/or CDR amino acid sequences with, e.g., 10 or fewer, 8 or fewer, 6 or fewer, 4 or fewer, etc. conservative amino acid substitutions relative to any of the HCVR, LCVR and/or CDR amino acid sequences disclosed herein. In some embodiments, the disclosure includes use of an anti-BCMA×CD3 antibody having HCVR, LCVR and/or CDR amino acid sequences with 1, 2, 3, or 4 conservative amino acid substitutions relative to any of the HCVR, LCVR and/or CDR amino acid sequences disclosed herein.

Other anti-BCMA×CD3 antibodies that can be used in the methods of the present disclosure include, e.g., the antibodies referred to and known in the art as pacanalotamab (AMG420), teclistamab (JNJ-64007957), AMG701, alnuctamab (CC-93269), EM801, EM901, elranatamab (PF-06863135), TNB383B (ABBV-383), and TNB384B, or any of the anti-BCMA×CD3 antibodies set forth, e.g., in WO 2013/072415, WO 2014/140248, WO 2014/122144, WO 2016/166629, WO 2016/079177, WO 2016/020332, WO 2017/031104, WO 2017/223111, WO 2017/134134, WO 2018/083204, or WO 2018/201051. The portions of the publications cited herein that identify anti-BCMA×CD3 antibodies are hereby incorporated by reference.

In some embodiments, the CDRs disclosed herein are identified according to the Kabat definition. In some embodiments, the CDRs are identified according to the Chothia definition. In some embodiments, the CDRs are identified according to the AbM definition. In some embodiments, the CDRs are identified according to the IMGT definition.

The bispecific antigen-binding molecules disclosed herein may be bispecific antibodies. In some cases, the bispecific antibody comprises a human IgG heavy chain constant region. In some cases, the human IgG heavy chain constant region is isotype IgG1. In some cases, the human IgG heavy chain constant region is isotype IgG4. In some embodiments, the human IgG heavy chain constant region comprises one or more modifications that increase binding to a neonatal Fc receptor (FcRn). In some embodiments, the human IgG heavy chain constant region comprises one or more modifications that decrease binding to an Fc-gamma receptor (FcγR).

In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In some embodiments, the antibody comprises a first heavy chain containing the HCVR of the first antigen-binding domain and a second heavy chain containing the HCVR of the second antigen-binding domain, wherein the first heavy chain comprises residues 1-450 of the amino acid sequence of SEQ ID NO: 41 and the second heavy chain comprises residues 1-449 of the amino acid sequence of SEQ ID NO: 42.

In some embodiments, the antibody comprises a common light chain containing the LCVR of the first and second antigen-binding domains, wherein the common light chain comprises the amino acid sequence of SEQ ID NO: 43.

In some embodiments, the anti-BCMA×CD3 bispecific antibody comprises a first heavy chain comprising the amino acid sequence of SEQ ID NO: 41, a second heavy chain comprising the amino acid sequence of SEQ ID NO: 42, and a common light chain comprising the amino acid sequence of SEQ ID NO: 43. In some cases, the mature form of the antibody may not include the C-terminal lysine residues of SEQ ID NOS: 41 and 42. Thus, in some cases the anti-BCMA binding arm comprises a heavy chain comprising residues 1-450 of SEQ ID NO: 41, and the anti-CD3 binding arm comprises a heavy chain comprising residues 1-449 of SEQ ID NO: 42.

The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

In some embodiments, a bispecific antigen-binding molecule of the present disclosure comprises two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In some embodiments, the multimerizing domain is an Fc fragment or an amino acid sequence of from 1 to about 200 amino acids in length containing at least one cysteine residue. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

### III. B Cell Depleting Agents

In some embodiments, the methods disclosed herein include administering a therapeutically effective amount of a B cell depleting agent to a subject in need thereof. As used herein, a “B cell depleting agent” refers to any molecule capable of specifically binding to a surface antigen on B cells and killing or depleting said B cell. Thus, in general, a B cell depleting agent can be any agent that binds to a B cell surface molecule. In some embodiments, the B cell depleting agent is capable of depleting B cells and plasma cells that express low levels of BCMA.

In various aspects, the present disclosure provides B cell depleting agents, which may be administered to a subject in need thereof, e.g., either alone or combined with, or administered in combination with, a plasma cell depleting agent (e.g., an anti-BCMA×CD3 bispecific antibody, or a functional fragment thereof), an immunoglobulin depleting agent (e.g., an FcRn blocker such as, e.g., efgartigimod), and/or an immunogen (e.g., a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein such as, e.g., AAV (e.g., AAV comprising a nucleic acid construct described herein). In some embodiments, plasmapheresis, therapeutic plasma exchange, and/or immunoadsorption may be further combined with the administering of the B cell depleting agent, the plasma cell depleting agent, the immunoglobulin depleting agent, and/or the immunogen. In some embodiments, the subject does not have a pre-existing immunity against the immunogen. For example, in some embodiments, the subject does not have a pre-existing immunity against a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein. In some such methods in which the subject does not have a pre-existing immunity against the immunogen (e.g., immunogenic delivery vehicle such as, e.g., AAV), the methods do not comprise administering a plasma cell depleting agent (the B cell depleting agent is not used in combination with a plasma cell depleting agent). In some such methods in which the subject does not have a pre-existing immunity against the immunogen (e.g., immunogenic delivery vehicle such as, e.g., AAV), the methods do not comprise administering an immunoglobulin depleting agent (the B cell depleting agent is not used in combination with an immunoglobulin depleting agent).

In some embodiments, a B cell depleting agent may be administered alone (e.g., as a monotherapy, in the absence of the administration of any other additional immunomodulators [e.g., plasma cell depleting agents, immunoglobulin depleting agents], and optionally, combined with, or administered in combination with, an immunogen) to a subject in need thereof, e.g., a subject without a pre-existing immunity against an immunogen (e.g., an immunogen to be administered to the subject e.g., an immunogenic delivery vehicle such as, e.g., AAV). For example, the subject may be a subject without a pre-existing immunity against a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein. In some embodiments, the B cell depleting agent may be administered alone to a subject who is immunologically naïve to an immunogen to be administered to the subject (e.g., AAV). In some embodiments, the B cell depleting agent may be administered alone to an AAV seronegative subject, and the subject is further administered an immunogen (e.g., AAV). In some embodiments, a B cell depleting agent may be useful as a prophylactic treatment to prevent or suppress an immune response (e.g., an anti-AAV IgG, IgM, and/or nAb response) to an immunogen (e.g., AAV) in a subject in need thereof (e.g., a subject without a pre-existing immunity to the immunogen).

In some embodiments, the suppression or prevention of an immune response (e.g., an anti-AAV IgG, IgM, and/or nAb response) to an immunogen in a subject (e.g., a subject without a pre-existing immunity to the immunogen) can be achieved by administering a B cell depleting agent described herein (e.g., an anti-CD20×CD3 bispecific antibody or a functional fragment thereof). Administration of the B cell depleting agent to the subject can suppress or prevent the immune response in the subject following the initial dosing and/or re-dosing of an immunogen (e.g., post-AAV dosing and/or re-dosing). In some embodiments, an immune response may be suppressed in a subject following AAV dosing and/or re-dosing. The immune response may be suppressed by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more, e.g., relative to an immune response in a subject receiving no immunomodulation treatment or treatment with a conventional anti-CD20 therapeutic alone (e.g., rituximab, or derivatives or equivalents thereof). The immune response may be suppressed by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The immune response may be suppressed by about 50%, about 55%, about 60%, about 65%, about 70%, about 75%, about 80%, about 85%, about 90%, about 91%, about 92%, about 93%, about 94%, about 95%, about 96%, about 97%, about 98%, about 99%, or even 100%. In some embodiments, the immune response is prevented. In some embodiments, an immune response may be suppressed or prevented in a subject following AAV dosing and/or re-dosing in the subject to achieve levels equivalent to, or even below, those of an AAV-naïve subject. In some embodiments, a B cell depleting agent is sufficient to enable effective re-dosing of an immunogen to a subject. The B cell depleting agent can be administered to the subject prior to the re-dosing of the immunogen any number of times and can be used to maintain a suppressed immune response to the immunogen in the subject for any period of time thereafter.

In some embodiments, a B cell depleting agent is capable of suppressing an anti-immunogen response (e.g., an anti-AAV response) in a subject, and the anti-immunogen response is mounted by the subject in response to repeated doses of the immunogen (e.g., AAV).

It is contemplated that a B cell depleting agent may be used in the suppression or prevention of an anti-immunogen antibody response (e.g., an anti-AAV antibody response) in a subject, and the suppression or prevention of the anti-immunogen antibody response involves B cell depletion in primary and/or secondary lymphoid tissues and non-lymphoid tissue as well, such as B cell aggregates forming in liver and muscle after AAV administration. Non-limiting examples of primary lymphoid tissues include bone marrow and thymus. In some embodiments, the compositions and methods of the disclosure encompass B cell depletion in secondary lymphoid tissues, for example, and without limitation, spleen and/or lymph nodes. In some embodiments, the compositions and methods of the disclosure relate to B cell depletion in lymph nodes, which is achieved by a B cell depleting agent described herein.

In some embodiments, the present disclosure provides B cell depleting agents combined with, or administered in combination with, plasma cell depleting agents (e.g., an anti-BCMA×CD3 bispecific antibody, or a functional fragment thereof) described herein to subjects, e.g., subjects with or without a pre-existing immunity against an immunogen (i.e., an immunogen administered to the subject, e.g., an immunogenic delivery vehicle such as, e.g., AAV). In some embodiments, the B cell depleting agent may be administered in combination with a plasma cell depleting agent, an immunoglobulin depleting agent, plasmapheresis, therapeutic plasma exchange, immunoadsorption, and/or an immunogen (e.g., an immunogenic delivery vehicle) disclosed herein. In some embodiments, the B cell depleting agent may be administered to subjects without a pre-existing immunity against an immunogen (i.e., an immunogen to be administered to the subject, e.g., an immunogenic delivery vehicle such as, e.g., AAV) not only alone, but also in combination with a plasma cell depleting agent, an immunoglobulin depleting agent, plasmapheresis, therapeutic plasma exchange, or immunoadsorption, and/or an immunogen (e.g., nucleic acid construct, nuclease agent or CRISPR/Cas system, e.g., in an immunogenic delivery vehicle) (e.g., an immunogenic delivery vehicle such as, e.g., AAV) disclosed herein. In some embodiments, the B cell depleting agent may be administered to subjects with a pre-existing immunity against an immunogen (i.e., an immunogen to be administered to the subject, e.g., an immunogenic delivery vehicle such as, e.g., AAV) in combination with a plasma cell depleting agent, an immunoglobulin depleting agent, plasmapheresis, therapeutic plasma exchange, or immunoadsorption, and/or an immunogen (e.g., nucleic acid construct, nuclease agent or CRISPR/Cas system, e.g., in an immunogenic delivery vehicle) (e.g., an immunogenic delivery vehicle such as, e.g., AAV) disclosed herein. Suitable combinations comprising a plasma cell depleting agent are described in more detail elsewhere herein.

In some embodiments, the B cell depleting agent is an agent that directly targets a B cell, e.g., an agent that binds to a B cell surface molecule. In some embodiments, the B cell depleting agent causes a reduction in the number of B cells in a subject (e.g., in a blood sample taken from the subject). In some embodiments, a B cell depleting agent may be useful for, e.g., eliminating non-plasma cell (e.g., non-long-lived plasma cell [LLPC] sources of immunogen (e.g., anti-AAV) nAbs. In some embodiments, a B cell depleting agent may be useful for, e.g., preventing formation of non-plasma cell (e.g., non-long-lived plasma cell [LLPC] sources of immunogen (e.g., anti-AAV) nAbs (e.g., in AAV-naïve patients). In some embodiments, the B cell depleting agent may capture a wider range of AAV-specific B cells and plasma cells that may not express high levels of BCMA (e.g., committed memory B cells and early plasmablasts).

In some embodiments, the B cell depleting agent comprises an anti-CD19 antibody (e.g., MEDI-551, tefasitamab, Inebilizumab, loncastuximab), an anti-CD20 antibody (e.g., rituximab, ocrelizumab, obinutuzumab, ublituximab, or ofatumumab), an anti-CD22 antibody (e.g., epratuzumab), an anti-CD79 antibody (e.g., polatuzumab), a bispecific anti-CD20×CD3 B cell depleting antibody (e.g. odronextamab, glofitamab, mosunetuzumab, epcoritamab), a bispecific anti-CD19×CD3 antibody (e.g., blinatumomab), a bispecific anti-CD22×CD3 antibody (e.g., inotuzumab), or functional fragments thereof, or any combination thereof.

In some embodiments, the B cell depleting agent is an agent that indirectly targets a B cell, e.g., by targeting a B cell survival factor. In some embodiments, the B cell depleting agent is a BLyS/BAFF inhibitor (e.g., belimumab, lanalumab, BR3-Fc, AMG-570, or AMG-623), an APRIL inhibitor (e.g., telitacicept, atacicept), or a BLyS receptor 3/BAFF receptor inhibitor (e.g., anti-BR3), or any combination thereof.

In some embodiments, the B cell depleting agent is selected from anti-CD19 antibodies, anti-CD20 antibodies, anti-CD22 antibodies, anti-CD79 antibodies, multispecific antibodies combining two or more of any of said antibody specificities, multispecific antibodies combining any of said antibody specificities with anti-CD3 antibodies, functional fragments of any of said antibodies, and any combinations thereof. In certain embodiments, the B cell depleting agent is an anti-CD20 antibody or a functional fragment thereof. In some embodiments, a multispecific anti-CD20 antibody or functional fragment thereof of the present disclosure targets CD20 and CD19. In some embodiments, the multispecific anti-CD20 antibody or functional fragment thereof is anti-CD19×CD20 bispecific antibody, or functional fragment thereof. In some embodiments, the B cell depleting agent comprises an anti-CD19 antibody and an anti-CD20 antibody.

In some embodiments, the B cell depleting agent comprises anti-CD19 and anti-CD20 antibodies (also referred to as “anti-CD19/CD20 antibodies” herein), or functional fragments thereof, disclosed herein.

In a specific embodiment, the B cell depleting agent comprises a bispecific antibody that specifically binds CD3 and CD19. Such antibodies may be referred to herein as, e.g., “anti-CD19/anti-CD3,” or “anti-CD19×CD3” or “CD19×CD3” bispecific antibodies, or other similar terminology.

In a specific embodiment, the B cell depleting agent comprises a bispecific antibody that specifically binds CD3 and CD20. Such antibodies may be referred to herein as, e.g., “anti-CD20/anti-CD3,” or “anti-CD20×CD3” or “CD20×CD3” bispecific antibodies, or other similar terminology.

As used herein, the expression “bispecific antibody” refers to an immunoglobulin protein comprising at least a first antigen-binding domain and a second antigen-binding domain. In some embodiments, the first antigen-binding domain specifically binds a first antigen (e.g., CD20), and the second antigen-binding domain specifically binds a second, distinct antigen (e.g., CD3). Each antigen-binding domain of a bispecific antibody comprises a heavy chain variable domain (HCVR) and a light chain variable domain (LCVR), each comprising three CDRs. In the context of a bispecific antibody, the CDRs of the first antigen-binding domain may be designated with the prefix “A” and the CDRs of the second antigen-binding domain may be designated with the prefix “B.” Thus, the CDRs of the first antigen-binding domain may be referred to herein as A-HCDR1, A-HCDR2, and A-HCDR3; and the CDRs of the second antigen-binding domain may be referred to herein as B-HCDR1, B-HCDR2, and B-HCDR3.

The first antigen-binding domain and the second antigen-binding domain can each be connected to a separate multimerizing domain. As used herein, a “multimerizing domain” is any macromolecule, protein, polypeptide, peptide, or amino acid that has the ability to associate with a second multimerizing domain of the same or similar structure or constitution. In the context of the present invention, the multimerizing component is an Fc portion of an immunoglobulin (comprising a C_{H}2-C_{H}3 domain), e.g., an Fc domain of an IgG selected from the isotypes IgG1, IgG2, IgG3, and IgG4, as well as any allotype within each isotype group.

Bispecific antibodies of the present invention typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats).

In the context of bispecific antibodies of the present invention, Fc domains may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and FcRn. In one embodiment, the bispecific antigen-binding molecule comprises a modification in a C_{H}2 or a C_{H}3 region, wherein the modification increases the affinity of the Fc domain to FcRn in an acidic environment (e.g., in an endosome where pH ranges from about 5.5 to about 6.0). Non-limiting examples of such Fc modifications are disclosed in US 2015/0266966, incorporated herein in its entirety.

The present invention also includes bispecific antibodies comprising a first C_{H}3 domain and a second Ig C_{H}3 domain, wherein the first and second Ig C_{H}3 domains differ from one another by at least one amino acid, and wherein at least one amino acid difference reduces binding of the bispecific antibody to Protein A as compared to a bi-specific antibody lacking the amino acid difference. In one embodiment, the first Ig C_{H}3 domain binds Protein A and the second Ig C_{H}3 domain contains a mutation that reduces or abolishes Protein A binding such as an H95R modification (by IMGT exon numbering; H435R by EU numbering). The second C_{H}3 may further comprise a Y96F modification (by IMGT; Y436F by EU). Further modifications that may be found within the second C_{H}3 include: D16E, L18M, N44S, K52N, V57M, and V821 (by IMGT; D356E, L358M, N384S, K392N, V397M, and V4221 by EU) in the case of IgG1 antibodies; N44S, K52N, and V821 (IMGT; N384S, K392N, and V4221 by EU) in the case of IgG2 antibodies; and Q15R, N44S, K52N, V57M, R69K, E79Q, and V821 (by IMGT; Q355R, N384S, K392N, V397M, R409K, E419Q, and V4221 by EU) in the case of IgG4 antibodies.

In certain embodiments, the Fc domain may be chimeric, combining Fc sequences derived from more than one immunoglobulin isotype. For example, a chimeric Fc domain can comprise part or all of a C_{H}2 sequence derived from a human IgG1, human IgG2 or human IgG4 C_{H}2 region, and part or all of a C_{H}3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 C_{H}1]-[IgG4 upper hinge]-[IgG2 lower hinge]-[IgG4 C_{H}2]-[IgG4 C_{H}3]. Another example of a chimeric Fc domain that can be included in any of the antigen-binding molecules set forth herein comprises, from N- to C-terminus: [IgG1 C_{H}1]-[IgG1 upper hinge]-[IgG2 lower hinge]-[IgG4 C_{H}2][IgG1 C_{H}3]. These and other examples of chimeric Fc domains that can be included in any of the antigen-binding molecules of the present invention are described in US Patent Publication No. 2014/0243504, which is herein incorporated in its entirety. Chimeric Fc domains having these general structural arrangements, and variants thereof, can have altered Fc receptor binding, which in turn affects Fc effector function.

### A. CD20×CD3 Antigen-Binding Molecules

The term “CD20,” as used herein, refers to an antigen which is expressed on B cells and which consists of a non-glycosylated phosphoprotein expressed on the cell membranes of mature B cells. The human CD20 protein can have the amino acid sequence as in NCBI Reference Sequence NP_690605.1. As used herein, the expression “anti-CD20 antibody” includes monovalent antibodies with a single specificity, such as RITUXAN® (rituximab), as described in U.S. Pat. No. 7,879,984. Exemplary anti-CD20 antibodies are also described in U.S. Pat. No. 7,879,984 and PCT International Application No. PCT/US2013/060511, filed on Sep. 19, 2013, each incorporated by reference herein.

In some exemplary embodiments, the CD20 targeting agent used in the disclosed methods is a multispecific (e.g., bispecific) antibody, or a functional fragment thereof, that specifically binds CD20 and CD3 (e.g., an anti-CD20×CD3 bispecific antibody). The anti-CD20×CD3 multispecific (e.g., bispecific) antibodies are useful for specific targeting and T-cell-mediated killing of cells that express CD20. The terms “antibody,” “antigen-binding fragment,” “human antibody,” “recombinant antibody,” and other related terminology are defined above. In the context of anti-CD20×CD3 antibodies and antigen-binding fragments thereof, the present disclosure includes the use of bispecific antibodies wherein one arm of an immunoglobulin is specific for CD20 or a fragment thereof, and the other arm of the immunoglobulin is specific for a second therapeutic target (e.g., CD3 on T-cells). Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mabe bispecific formats (see, e.g., Klein et al. 2012, mAbs 4(6):653-663, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc., 2013, 135(1):340-46).

The anti-CD20×CD3 bispecific antibodies are capable of simultaneously binding to human CD3 and human CD20. According to certain embodiments, the anti-CD20×CD3 bispecific antibodies specifically interact with cells that express CD3 and/or CD20. The extent to which the anti-CD20×CD3 bispecific antibodies binds cells that express CD3 and/or CD20 can be assessed by fluorescence activated cell sorting (FACS). In certain embodiments, the anti-CD20×CD3 bispecific antibodies specifically bind human T-cell lines which express CD3 (e.g., Jurkat), human B-cell lines which express CD20 (e.g., Raji), and primate T-cells (e.g., cynomolgus peripheral blood mononuclear cells [PBMCs]).

In some embodiments, the anti-CD20×CD3 bispecific antigen-binding molecule comprises a first antigen-binding domain (D1) that binds an epitope of CD20 (e.g., human CD20), and a second antigen-binding domain (D2) that binds an epitope of CD3 (e.g., human CD3).

According to certain exemplary embodiments of the present invention, the bispecific anti-CD20×CD3 antibody, or antigen-binding fragment thereof comprises heavy chain variable regions (A-HCVR and B-HCVR), light chain variable region (LCVR), and/or complementarity determining regions (CDRs) comprising any of the amino acid sequences of the bispecific anti-CD20×CD3 antibodies as set forth in US Patent Publication No. 20150266966, incorporated herein by reference in its entirety for all purposes. In certain exemplary embodiments, the bispecific anti-CD20×CD3 antibody or antigen-binding fragment thereof that can be used in the context of the methods of the present invention comprises: (a) a first antigen-binding arm comprising the heavy chain complementarity determining regions (A-HCDR1, A-HCDR2 and A-HCDR3) of a heavy chain variable region (A-HCVR) comprising the amino acid sequence of SEQ ID NO: 44 and the light chain complementarity determining regions (LCDRs) of a light chain variable region (LCVR) comprising the amino acid sequence of SEQ ID NO: 45; and (b) a second antigen-binding arm comprising the heavy chain CDRs (B-HCDR1, B-HCDR2 and B-HCDR3) of a HCVR (B-HCVR) comprising the amino acid sequence of SEQ ID NO: 46 and the light chain CDRs of a LCVR comprising the amino acid sequence of SEQ ID NO: 45. According to certain embodiments, the A-HCDR1 comprises the amino acid sequence of SEQ ID NO: 47; the A-HCDR2 comprises the amino acid sequence of SEQ ID NO: 48; the A-HCDR3 comprises the amino acid sequence of SEQ ID NO: 49; the LCDR1 comprises the amino acid sequence of SEQ ID NO: 50; the LCDR2 comprises the amino acid sequence of SEQ ID NO: 51; the LCDR3 comprises the amino acid sequence of SEQ ID NO: 52; the B-HCDR1 comprises the amino acid sequence of SEQ ID NO: 53; the B-HCDR2 comprises the amino acid sequence of SEQ ID NO: 54; and the B-HCDR3 comprises the amino acid sequence of SEQ ID NO: 55. In yet other embodiments, the bispecific anti-CD20×CD3 antibody or antigen-binding fragment thereof comprises: (a) a first antigen-binding arm comprising a HCVR (A-HCVR) comprising SEQ ID NO: 44 and a LCVR comprising SEQ ID NO: 45; and (b) a second antigen-binding arm comprising a HCVR (B-HCVR) comprising SEQ ID NO: 46 and a LCVR comprising SEQ ID NO: 45.

In some embodiments, the anti-CD20×CD3 bispecific antibody or functional fragment thereof comprises a first antigen-binding domain that specifically binds to CD20 comprising three heavy chain CDRs (HCDR1, HCDR2 and HCDR3) contained within a heavy chain variable region (HCVR) comprising the amino acid sequence of SEQ ID NO: 44, and three light chain CDRs (LCDR1, LCDR2 and LCDR3) contained within a light chain variable region (LCVR) comprising the amino acid sequence of SEQ ID NO: 45.

In some embodiments, the first antigen-binding domain that specifically binds to CD20 comprises a HCDR1 comprising the amino acid sequence of SEQ ID NO: 47, a HCDR2 comprising the amino acid sequence of SEQ ID NO: 48, a HCDR3 comprising the amino acid sequence of SEQ ID NO: 49, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises a second antigen-binding domain that specifically binds to CD3 comprising three heavy chain CDRs (HCDR1, HCDR2 and HCDR3) contained within a heavy chain variable region (HCVR) comprising the amino acid sequence of SEQ ID NO: 46, and three light chain CDRs (LCDR1, LCDR2 and LCDR3) contained within a light chain variable region (LCVR) comprising the amino acid sequence of SEQ ID NO: 45.

In some embodiments, the second antigen-binding domain that specifically binds to CD3 comprises a HCDR1 comprising the amino acid sequence of SEQ ID NO: 53, a HCDR2 comprising the amino acid sequence of SEQ ID NO: 54, a HCDR3 comprising the amino acid sequence of SEQ ID NO: 55, a LCDR1 comprising the amino acid sequence of SEQ ID NO: 50, a LCDR2 comprising the amino acid sequence of SEQ ID NO: 51, and a LCDR3 comprising the amino acid sequence of SEQ ID NO: 52.

In some embodiments, the anti-CD20×CD3 bispecific antibody or functional fragment thereof comprises: a first antigen-binding domain that comprises HCDR1, HCDR2, and HCDR3 comprising the amino acid sequences of SEQ ID NOS: 47, 48, and 49, respectively, and LCDR1, LCDR2, and LCDR3 comprising the amino acid sequences of SEQ ID NOS: 50, 51, and 52, respectively; and a second antigen-binding domain that comprises HCDR1, HCDR2, and HCDR3 comprising the amino acid sequences of SEQ ID NOS: 53, 54, and 55, respectively, and LCDR1, LCDR2, and LCDR3 comprising the amino acid sequences of SEQ ID NOS: 50, 51, and 52, respectively.

Other bispecific anti-CD20×CD3 antibodies that can be used in the context of the methods of the present invention include, e.g., any of the antibodies as set forth in US 2014/0088295, US 2015/0166661, and US 2017/0174781, each of which is herein incorporated by reference in its entirety for all purposes. An exemplary bispecific anti-CD20×CD3 antibody that can be used in the context of the methods of the present invention is the bispecific anti-CD20×CD3 antibody known as REGN1979 or bsAB1.

In some exemplary embodiments, an anti-CD20×CD3 bispecific antibody or antigen-binding fragment thereof that can be used in the context of the present disclosure comprising a HCVR, a LCVR, and/or CDRs comprising the amino acid sequences of REGN1979 as set forth in Table 2 below.

TABLE 2
Amino Acid Sequences of Exemplary Anti-CD20xCD3 Bispecific
Antibodies.

| | Anti-CD20 | | | | Anti-CD3 | | | | Common | | | |
|---|---|---|---|---|---|---|---|---|---|---|---|---|
| Bispecific | First Antigen-Binding | | | | Second Antigen-Binding | | | | Light Chain Variable | | | |
| antibody | Domain | | | | Domain | | | | Region | | | |
| identifier | HCVR | HCDR1 | HCDR2 | HCDR3 | HCVR | HCDR1 | HCDR2 | HCDR3 | LCVR | LCDR1 | LCDR2 | LCDR3 |
| REGN1979 | 44 | 47 | 48 | 49 | 46 | 53 | 54 | 55 | 45 | 50 | 51 | 52 |

Anti-CD20 HCVR Protein Sequence
SEQ ID NO: 44
EVQLVESGGGLVQPGRSLRLSCVASGFTENDYAMHWVRQAPGKGL
EWVSVISWNSDSIGYADSVKGRFTISRDNAKNSLYLQMHSLRAED
TALYYCAKDNHYGSGSYYYYQYGMDVWGQGTTVTVSS
Common LCVR Protein Sequence
SEQ ID NO: 45
EIVMTQSPATLSVSPGERATLSCRASQSVSSNLAWYQQKPGQAPR
LLIYGASTRATGIPARFSGSGSGTEFTLTISSLQSEDFAVYYCQH
YINWPLTFGGGTKVEIKR
Anti-CD3 HCVR Protein Sequence
SEQ ID NO: 46
EVQLVESGGGLVQPGRSLRLSCAASGFTEDDYTMHWVRQAPGKGL
EWVSGISWNSGSIGYADSVKGRFTISRDNAKKSLYLQMNSLRAED
TALYYCAKDNSGYGHYYYGMDVWGQGTTVTVAS
Anti-CD20 HCDR1 Protein Sequence
SEQ ID NO: 47
GFTENDYA
Anti-CD20 HCDR2 Protein Sequence
SEQ ID NO: 48
ISWNSDSI
Anti-CD20 HCDR3 Protein Sequence
SEQ ID NO: 49
AKDNHYGSGSYYYYQYGMDV
Common LCDR1 Protein Sequence
SEQ ID NO: 50
QSVSSN
Common LCDR2 Protein Sequence
SEQ ID NO: 51
GAS
Common LCDR3 Protein Sequence
SEQ ID NO: 52
QHYINWPLT
Anti-CD3 HCDR1 Protein Sequence
SEQ ID NO: 53
GFTFDDYT
Anti-CD3 HCDR2 Protein Sequence
SEQ ID NO: 54
ISWNSGSI
Anti-CD3 HCDR3 Protein Sequence
SEQ ID NO: 55
AKDNSGYGHYYYGMDV

In some embodiments, the anti-CD20×CD3 bispecific antibody or antigen-binding fragment thereof that can be used in the present disclosure comprises: (a) a first antigen binding domain that binds specifically to CD20; and (b) a second antigen-binding domain that binds specifically to CD3. In one embodiment, the anti-CD20 antigen-binding domain comprises the heavy chain complementarity determining regions (A-HCDRs) of a heavy chain variable region (A-HCVR) comprising the amino acid sequence of SEQ ID NO: 44 and the light chain complementarity determining regions (LCDRs) of a light chain variable region (LCVR) comprising the amino acid sequence of SEQ ID NO: 45. In one embodiment, the first antigen-binding domain comprises three HCDRs (A-HCDR1, A-HCDR2 and A-HCDR3) and three LCDRs (LCDR1, LCDR2 and LCDR3), wherein the A-HCDR1 comprises the amino acid sequence of SEQ ID NO: 47; the A-HCDR2 comprises the amino acid sequence of SEQ ID NO: 48; the A-HCDR3 comprises the amino acid sequence of SEQ ID NO: 49; the LCDR1 comprises the amino acid sequence of SEQ ID NO: 50; the LCDR2 comprises the amino acid sequence of SEQ ID NO: 51; and the LCDR3 comprises the amino acid sequence of SEQ ID NO: 52.

In one embodiment, the second antigen-binding domain comprises the heavy chain complementarity determining regions (B-HCDRs) of a heavy chain variable region (B-HCVR) comprising the amino acid sequence of SEQ ID NO: 46 and the light chain complementarity determining regions (LCDRs) of a light chain variable region (LCVR) comprising the amino acid sequence of SEQ ID NO: 45. In one embodiment, the second antigen-binding domain comprises three HCDRs (B-HCDR1, B-HCDR2 and B-HCDR3) and three LCDRs (LCDR1, LCDR2 and LCDR3), wherein the B-HCDR1 comprises the amino acid sequence of SEQ ID NO: 53; the B-HCDR2 comprises the amino acid sequence of SEQ ID NO: 54; the B-HCDR3 comprises the amino acid sequence of SEQ ID NO: 55; the LCDR1 comprises the amino acid sequence of SEQ ID NO: 50; the LCDR2 comprises the amino acid sequence of SEQ ID NO: 51; and the LCDR3 comprises the amino acid sequence of SEQ ID NO: 52.

In one embodiment, the anti-CD20×CD3 bispecific antibody or antigen-binding fragment thereof comprises: (a) a first antigen-binding domain that comprises A-HCDR1, A-CDR2, and A-HCDR3 domains, respectively, comprising the amino acid sequences of SEQ ID NOs: 47, 48, and 49, and LCDR1, LCDR2, and LCDR3 domains, respectively, comprising the amino acid sequences of SEQ ID NOs: 50, 51, and 52; and (b) a second antigen binding domain that comprises B-HCDR1, B-HCDR2, and B-HCDR3 domains, respectively, comprising the amino acid sequences of SEQ ID NOs: 53, 54, and 55, and LCDR1, LCDR2, and LCDR3 domains, respectively, comprising the amino acid sequences of SEQ ID NOs: 50, 51, and 52. In one embodiment, the anti-CD20×CD3 bispecific antibody or antigen-binding fragment thereof comprises: (a) a first antigen-binding domain that comprises a A-HCVR comprising the amino acid sequence of SEQ ID NO: 44 and a LCVR comprising the amino acid sequence of SEQ ID NO: 45; and (b) a second antigen-binding domain that comprises a B-HCVR comprising the amino acid sequence of SEQ ID NO: 46 and a LCVR comprising the amino acid sequence of SEQ ID NO: 45.

Exemplary anti-CD20×CD3 bispecific antibodies include the fully human bispecific antibody known as REGN1979. See, e.g., US 2014/0088295, US 2015/0166661, and US 2017/0174781, each of which is herein incorporated by reference. According to certain exemplary embodiments, the methods of the present disclosure comprise the use of REGN1979, or a bioequivalent thereof. As used herein, the term “bioequivalent” with respect to anti-CD20×CD3 antibodies refers to antibodies or CD20×CD3 binding proteins or fragments thereof that are pharmaceutical equivalents or pharmaceutical alternatives having a rate and/or extent of absorption that does not show a significant difference with that of a reference antibody (e.g., REGN1979) when administered at the same molar dose under similar experimental conditions, either single dose or multiple dose; the term “bioequivalent” also includes antigen-binding proteins that bind to CD20/CD3 and do not have clinically meaningful differences with the reference antibody (e.g., REGN1979) with respect to safety, purity, and/or potency.

In some embodiments, the anti-CD20×CD3 bispecific antibody or antigen-binding fragment thereof comprises: (a) a first antigen-binding domain that comprises a A-HCVR having at least 90%, 91%, 92%, 93%, 94%, 95%, 96%, 97%, 98%, or 99% sequence identity to the amino acid sequence of SEQ ID NO: 44 and a LCVR having at least 90%, 91%, 92%, 93%, 94%, 95%, 96%, 97%, 98%, or 99% sequence identity to the amino acid sequence of SEQ ID NO: 45; and (b) a second antigen-binding domain that comprises a B-HCVR having at least 90%, 91%, 92%, 93%, 94%, 95%, 96%, 97%, 98%, or 99% sequence identity to the amino acid sequence of SEQ ID NO: 46 and a LCVR having at least 90%, 91%, 92%, 93%, 94%, 95%, 96%, 97%, 98%, or 99% sequence identity to the amino acid sequence of SEQ ID NO: 45. In some embodiments, the anti-CD20×CD3 bispecific antibody or antigen-binding fragment thereof comprises: (a) a first antigen-binding domain that comprises three HCDRs (A-HCDR1, A-HCDR2 and A-HCDR3) comprising the amino acid sequences of SEQ ID NOs: 47, 48, and 49, respectively, and an A-HCVR having at least 90%, 91%, 92%, 93%, 94%, 95%, 96%, 97%, 98%, or 99% sequence identity to the amino acid sequence of SEQ ID NO: 44, and comprises three LCDRs (LCDR1, LCDR2 and LCDR3) comprising the amino acid sequences of SEQ ID NOs: 50, 51, and 52, respectively, and a LCVR having at least 90%, 91%, 92%, 93%, 94%, 95%, 96%, 97%, 98%, or 99% sequence identity to the amino acid sequence of SEQ ID NO: 45; and (b) a second antigen-binding domain that comprises three HCDRs (B-HCDR1, B-HCDR2 and B-HCDR3) comprising the amino acid sequences of SEQ ID NOs: 53, 54, and 55, respectively, and a B-HCVR having at least 90%, 91%, 92%, 93%, 94%, 95%, 96%, 97%, 98%, or 99% sequence identity to the amino acid sequence of SEQ ID NO: 46, and comprises three LCDRs (LCDR1, LCDR2 and LCDR3) comprising the amino acid sequences of SEQ ID NOs: 50, 51, and 52, respectively, and a LCVR having at least 90%, 91%, 92%, 93%, 94%, 95%, 96%, 97%, 98%, or 99% sequence identity to the amino acid sequence of SEQ ID NO:45.

The present disclosure also includes variants of the anti-CD20×CD3 antibodies described herein comprising any of the HCVR, LCVR, and/or CDR amino acid sequences disclosed herein with one or more conservative amino acid substitutions. For example, the present disclosure includes use of anti-CD20×CD3 antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In some embodiments, the disclosure includes use of an anti-CD20×CD3 antibody having HCVR, LCVR, and/or CDR amino acid sequences with 1, 2, 3, or 4 conservative amino acid substitutions relative to any of the HCVR, LCVR, and/or CDR amino acid sequences disclosed herein.

In some embodiments, the CDRs disclosed herein are identified according to the Kabat definition. In some embodiments, the CDRs are identified according to the Chothia definition. In some embodiments, the CDRs are identified according to the AbM definition. In some embodiments, the CDRs are identified according to the IMGT definition.

In some embodiments, the anti-CD20×CD3 bispecific antibody or functional fragment thereof comprises a human IgG heavy chain constant region. In some embodiments, the human IgG heavy chain constant region is isotype IgG4 or IgG1. In some embodiments, the human IgG heavy chain constant region comprises one or more modifications that increase binding to a neonatal Fc receptor (FcRn). In some embodiments, the human IgG heavy chain constant region comprises one or more modifications that decrease binding to an Fc-gamma receptor (FcγR).

### IV. Sequence Variants

The antigen-binding molecules of the present disclosure may comprise one or more amino acid substitutions, insertions, and/or deletions in the framework and/or CDR regions of the heavy and/or light chain variable domains as compared to the corresponding germline sequences from which the individual antigen-binding domains were derived. Such mutations can be readily ascertained by comparing the amino acid sequences disclosed herein to germ line sequences available from, for example, public antibody sequence databases. The antigen-binding molecules of the present disclosure may comprise antigen binding fragments which are derived from any of the exemplary amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antigen-binding domain was originally derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germ line sequence from which the antigen-binding domain was originally derived). Furthermore, the antigen-binding domains may contain any combination of two or more germline mutations within the framework and/or CDR regions, e.g., wherein certain individual residues are mutated to the corresponding residue of a particular germ line sequence while certain other residues that differ from the original germ line sequence are maintained or are mutated to the corresponding residue of a different germline sequence. Once obtained, antigen-binding domains that contain one or more germline mutations can be easily tested for one or more desired property such as, improved binding specificity, increased binding affinity, improved, or enhanced antagonistic or agonistic biological properties, reduced immunogenicity, etc. Bispecific antigen-binding molecules comprising one or more antigen-binding domains obtained in this general manner are encompassed within the present disclosure.

The present disclosure also includes antigen-binding molecules wherein one or both antigen-binding domains comprise variants of any of the HCVR, LCVR, and/or CDR amino acid sequences disclosed herein having one or more conservative substitutions. For example, the present disclosure includes antigen-binding molecules comprising an antigen-binding domain having HCVR, LCVR, and/or CDR amino acid sequences with, e.g., 10 or fewer, 9 or fewer, 8 or fewer, 7 or fewer, 6 or fewer, 5 or fewer, 4 or fewer, 3 or fewer, 2 or fewer, or 1 conservative amino acid substitution(s) relative to any of the HCVR, LCVR, and/or CDR amino acid sequences disclosed herein. In some embodiments, the disclosure includes use of an antibody having HCVR, LCVR and/or CDR amino acid sequences with 1, 2, 3, or 4 conservative amino acid substitutions relative to any of the HCVR, LCVR and/or CDR amino acid sequences disclosed herein. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

The present disclosure also includes antigen-binding molecules comprising an antigen binding domain with a HCVR, LCVR, and/or CDR amino acid sequence that is substantially identical to any of the HCVR, LCVR, and/or CDR amino acid sequences disclosed herein. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1. In some embodiments, an antigen-binding molecule comprises a HCVR, LCVR, and/or CDR amino acid sequence having at least 85% sequence identity, e.g., at least 90%, at least 91%, at least 92%, at least 93%, at least 94%, at least 95%, at least 96%, at least 97%, at least 98% or at least 99% sequence identity, to a sequence disclosed in Table 1, wherein the differences in the amino acid residue(s) relative to the sequence disclosed in Table 1 are conservative substitutions or moderately conservative substitutions.

### V. Antigen-Binding Proteins Comprising Fc Modifications

In some embodiments, an antigen-binding molecule as disclosed herein (e.g., a BCMA×CD3 bispecific antigen-binding molecule such as an anti-BCMA×CD3 bispecific antibody or a CD20×CD3 bispecific antigen-binding molecule such as an anti-CD20×CD3 bispecific antibody) comprises an Fc domain comprising one or more modifications or mutations that enhance or diminish antibody binding to the FcRn receptor. For example, the present disclosure includes antigen-binding molecules comprising one or more mutations in the CH2 and/or CH3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal.

Non-limiting examples of such Fc modifications include, e.g., a modification at position 250 (e.g., E or Q); 250 and 428 (e.g., L or F); 252 (e.g., UY/F/W or T), 254 (e.g., S or T), and 256 (e.g., S/R/Q/E/D or T); or a modification at position 428 and/or 433 (e.g., H/UR/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P). See, e.g., Ko et al., BioDrugs 2021, 35:147-157.

In certain embodiments, a BCMA×CD3 bispecific antigen-binding molecule or a CD20×CD3 bispecific antigen-binding molecule comprises an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). In some embodiments, the human IgG heavy chain constant region comprises one or more modifications that increase binding to a neonatal Fc receptor (FcRn). For example, in some embodiments the human IgG heavy chain constant region comprises M252Y, S254T, and T256E mutations.

In some embodiments, the BCMA×CD3 bispecific antigen-binding molecules or the CD20×CD3 bispecific antigen-binding molecules of the present disclosure comprise a modified Fc domain having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments, a modified Fc domain having reduced binding to an Fc receptor, such as an Fc-gamma receptor (e.g., Fcγ receptor, e.g., FcγRI, FcγRIIA, FcγRIIB, or FcγRIIIA), is a variant IgG1 Fc or a variant IgG4 Fc comprising one or more substitutions or modifications in the hinge region and/or a CH region (e.g., CH2). For example, a modified Fc domain may comprise a variant IgG1 Fc wherein at least one amino acid of an IgG1 Fc hinge region and/or CH region is replaced with the corresponding amino acid from an IgG2 Fc hinge region and/or CH region. In certain embodiments, the modified Fc domain is a variant IgG1 Fc or a variant IgG4 Fc comprising one or more substitutions or modifications in the hinge region. For example, a modified Fc domain may comprise a variant IgG1 Fc wherein at least one amino acid of the IgG1 Fc hinge region is replaced with the corresponding amino acid from the IgG2 Fc hinge region. In one example, the variant IgG1 Fc can comprise a human IgG2 lower hinge amino acid sequence or can comprise both a human IgG2 lower hinge amino acid sequence and a human IgG4 CH2 amino acid sequence. For example, in some embodiments, the heavy chain constant region can comprise a variant IgG1 Fc in which positions 233-236 by EU numbering are occupied by PVA. See, e.g., U.S. Pat. No. 10,988,537, the disclosure of which is hereby incorporated by reference in its entirety. In some embodiments, the heavy chain constant region can comprise a variant IgG1 Fc in which the IgG1 CH2 region is replaced with the corresponding amino acids from the IgG4 CH2 region and in which positions 233-236 by EU numbering are occupied by PVA. Alternatively, a modified Fc domain may comprise a variant IgG4 Fc wherein at least one amino acid of an IgG4 Fc hinge region and/or CH region is replaced with the corresponding amino acid from an IgG2 Fc hinge region and/or CH region. Alternatively, a modified Fc domain may comprise a variant IgG4 Fc wherein at least one amino acid of the IgG4 Fc hinge region is replaced with the corresponding amino acid from the IgG2 Fc hinge region. In one example, the variant IgG4 Fc can comprise a human IgG2 lower hinge amino acid sequence. For example, in some embodiments, the heavy chain constant region can comprise a variant IgG4 Fc in which positions 233-236 by EU numbering are occupied by PVA. See, e.g., U.S. Pat. No. 10,988,537, the disclosure of which is hereby incorporated by reference in its entirety. In some embodiments, a modified Fc domain comprises a modified hinge region in which each of positions 233-236 by EU numbering is occupied by G or is unoccupied. In some embodiments, a modified Fc domain comprises modifications in which each of positions 233-236 by EU numbering is occupied by G or is unoccupied. For example, in some embodiments, a modified Fc domain can comprise a modified hinge region in which positions 233-236 by EU numbering are occupied by GGG. See, e.g., U.S. Pat. No. 11,518,807, the disclosure of which is hereby incorporated by reference in its entirety. In some embodiments, the heavy chain constant region can comprise a variant IgG1 Fc in which the IgG1 CH2 region is replaced with the corresponding amino acids from the IgG4 CH2 region and in which positions 233-236 by EU numbering are occupied by GGG. Non-limiting, exemplary modified Fc regions that can be used in the context of the present disclosure are set forth in U.S. Pat. No. 11,518,807, the disclosure of which is hereby incorporated by reference in its entirety, as well as any functionally equivalent variants of the modified Fc regions set forth therein. Other modified Fc domains and Fc modifications that can be used in the context of the present disclosure include any of the modifications as set forth in U.S. Pat. Nos. 8,697,396, 10,988,537, US 2014/0171623, US 2014/0134162, US 2014/0243504, and WO 2014/043361, the disclosures of each of which are incorporated by reference herein.

All possible combinations of the foregoing Fc domain mutations, and other mutations within the antibody variable domains disclosed herein, are contemplated within the scope of the present disclosure.

### VI. Polynucleotides, Vectors, and Host Cells

In another aspect, the present disclosure provides nucleic acid molecules comprising one or more polynucleotide sequences encoding the antigen-binding molecules disclosed herein, as well as vectors (e.g., expression vectors) encoding such polynucleotide sequences and host cells into which such vectors have been introduced.

Polynucleotides, as disclosed herein, may encode all or a portion of an antigen-binding molecule, antibody, or antigen-binding fragment as disclosed throughout the present disclosure. In some cases, a single polynucleotide may encode both a HCVR and a LCVR (e.g., defined with reference to the CDRs contained within the respective amino acid sequence-defined HCVR and LCVR, defined with reference to the amino acid sequences of the CDRs of the HCVR and LCVR, respectively, or defined with reference to the amino acid sequences of the HCVR and LCVR, respectively) of an antibody or antigen-binding fragment, or the HCVR and LCVR may be encoded by separate polynucleotides (i.e., a pair of polynucleotides). In the latter case, in which the HCVR and LCVR are encoded by separate polynucleotides, the polynucleotides may be combined in a single vector or may be contained in separate vectors (i.e., a pair of vectors). In any case, a host cell used to express the polynucleotide(s) or vector(s) may contain the full complement of component parts to generate the antibody or antigen-binding fragment thereof. For example, a host cell may comprise separate vectors, each encoding a HCVR and a LCVR, respectively, of an antibody or antigen-binding fragment thereof as discussed above or herein. Similarly, the polynucleotide or polynucleotides, and the vector or vectors, may be used to express the full-length heavy chain and full-length light chain of an antibody as discussed above or herein. For example, a host cell may comprise a single vector with polynucleotides encoding both a heavy chain and a light chain of an antibody, or the host cell may comprise separate vectors with polynucleotides encoding, respectively, a heavy chain and a light chain of an antibody as disclosed above or herein.

In some embodiments, the nucleic acid molecule comprises one or more polynucleotide sequences encoding an antigen-binding molecule disclosed in Table 1.

In some embodiments, the nucleic acid molecule comprises a polynucleotide sequence that encodes an anti-BCMA HCVR comprising the HCDR1, HCDR2, and HCDR3 of SEQ ID NOS: 4, 6, and 8, respectively. In some embodiments, the nucleic acid molecule comprises a polynucleotide sequence that encodes an anti-BCMA HCVR comprising or consisting of the sequence of SEQ ID NO: 2. In some embodiments, the nucleic acid molecule comprises a polynucleotide sequence of SEQ ID NO: 1, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 1.

In some embodiments, the nucleic acid molecule comprises a polynucleotide sequence that encodes an anti-CD3 HCVR comprising the HCDR1, HCDR2, and HCDR3 of SEQ ID NOS: 28, 30, and 32, respectively; or of SEQ ID NOS: 36, 38, and 40, respectively. In some embodiments, the nucleic acid molecule comprises a polynucleotide sequence that encodes an anti-CD3 HCVR comprising or consisting of the sequence of SEQ ID NO: 26 or SEQ ID NO: 34. In some embodiments, the nucleic acid molecule comprises a polynucleotide sequence of SEQ ID NO: 25 or 33, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 25 or 33.

In some embodiments, the nucleic acid molecule comprises a polynucleotide sequence that encodes an LCVR comprising an LCDR1 comprising or consisting of the amino acid sequence of SEQ ID NO: 20, an LCDR2 comprising the amino acid sequence AAS (SEQ ID NO: 22), and an LCDR3 comprising the amino acid sequence of SEQ ID NO: 24. In some embodiments, the nucleic acid molecule comprises a polynucleotide sequence that encodes an LCVR comprising or consisting of the sequence of SEQ ID NO: 18. In some embodiments, the nucleic acid molecule comprises the polynucleotide sequence of SEQ ID NO: 17, or a polynucleotide sequence having at least 70% sequence identity, e.g., at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% sequence identity, to SEQ ID NO: 17.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding domain that binds BCMA, and a second nucleic acid molecule comprising a polynucleotide sequence encoding an HCVR and/or LCVR of a second antigen-binding domain that binds CD3. In some embodiments, a composition comprises a first nucleic acid molecule comprising a polynucleotide sequence encoding an HCVR of a first antigen-binding domain that binds BCMA, a second nucleic acid molecule comprising a polynucleotide sequence encoding an LCVR of a first antigen-binding domain that binds BCMA, a third nucleic acid molecule comprising a polynucleotide sequence encoding an HCVR of a second antigen-binding domain that binds CD3, and a fourth nucleic acid molecule comprising a polynucleotide sequence encoding an LCVR of a second antigen-binding domain that binds CD3. In some embodiments, an anti-BCMA HCVR comprises the HCDR1, HCDR2, and HCDR3 of SEQ ID NOS: 4, 6, and 8, respectively. In some embodiments, an anti-BCMA LCVR comprises LCDR1, LCDR2, and LCDR3 of SEQ ID NOS: 20, 22, and 24, respectively. In some embodiments, an anti-CD3 HCVR comprises the HCDR1, HCDR2, and HCDR3 of SEQ ID NOS: 28, 30, and 32, respectively; or the HCDR1, HCDR2, and HCDR3 of SEQ ID NOS: 36, 38, and 40, respectively. In some embodiments, an anti-CD3 LCVR comprises the LCDR1, LCDR2, and LCDR3 of SEQ ID NOS: 20, 22, and 24, respectively.

In one embodiment, the present disclosure provides a nucleic acid molecule or nucleic acid molecules that comprise a nucleotide sequence encoding the HCVR sequence of the anti-BCMA antigen-binding domain comprising SEQ ID NO: 2, a nucleotide sequence encoding the HCVR sequence of the anti-CD3 antigen-binding domain comprising SEQ ID NO: 26, and a nucleotide sequence encoding the LCVR sequence comprising SEQ ID NO: 18.

In one embodiment, the present disclosure provides a nucleic acid molecule or nucleic acid molecules that comprise a nucleotide sequence encoding the HCVR sequence of the anti-BCMA antigen-binding domain comprising SEQ ID NO: 2, a nucleotide sequence encoding the HCVR sequence of the anti-CD3 antigen-binding domain comprising SEQ ID NO: 34, and a nucleotide sequence encoding the LCVR sequence comprising SEQ ID NO: 18.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

In some embodiments, the nucleic acid molecule comprises one or more polynucleotide sequences encoding an antigen-binding molecule disclosed in Table 2.

In some embodiments, the nucleic acid molecule comprises a polynucleotide sequence that encodes an anti-CD20 HCVR comprising the HCDR1, HCDR2, and HCDR3 of SEQ ID NOs: 47, 48, and 49, respectively. In some embodiments, the nucleic acid molecule comprises a polynucleotide sequence that encodes an anti-CD20 HCVR comprising or consisting of the sequence of SEQ ID NO: 44.

In some embodiments, the nucleic acid molecule comprises a polynucleotide sequence that encodes an anti-CD3 HCVR comprising the HCDR1, HCDR2, and HCDR3 of SEQ ID NOs: 53, 54, and 55, respectively. In some embodiments, the nucleic acid molecule comprises a polynucleotide sequence that encodes an anti-CD3 HCVR comprising or consisting of the sequence of SEQ ID NO: 46.

In some embodiments, the nucleic acid molecule comprises a polynucleotide sequence that encodes an LCVR comprising the LCDR1, LCDR2, and LCDR3 of SEQ ID NOs: 50, 51, and 52, respectively. In some embodiments, the nucleic acid molecule comprises a polynucleotide sequence that encodes an LCVR comprising or consisting of the sequence of SEQ ID NO: 45.

In some embodiments, compositions are provided comprising one or more nucleic acid molecules as disclosed herein. For example, in some embodiments, a composition comprises a first nucleic acid molecule comprising a polynucleotide sequence encoding an HCVR and/or LCVR of a first antigen-binding domain that binds CD20, and a second nucleic acid molecule comprising a polynucleotide sequence encoding an HCVR and/or LCVR of a second antigen-binding domain that binds CD3. In some embodiments, a composition comprises a first nucleic acid molecule comprising a polynucleotide sequence encoding an HCVR of a first antigen-binding domain that binds CD20, a second nucleic acid molecule comprising a polynucleotide sequence encoding an LCVR of a first antigen-binding domain that binds CD20, a third nucleic acid molecule comprising a polynucleotide sequence encoding an HCVR of a second antigen-binding domain that binds CD3, and a fourth nucleic acid molecule comprising a polynucleotide sequence encoding an LCVR of a second antigen-binding domain that binds CD3. In some embodiments, an anti-CD20 HCVR comprises the HCDR1, HCDR2, and HCDR3 of SEQ ID NOs: 47, 48, and 49, respectively. In some embodiments, an anti-CD20 LCVR comprises LCDR1, LCDR2, and LCDR3 of SEQ ID NOs: 50, 51, and 52, respectively. In some embodiments, an anti-CD3 HCVR comprises the HCDR1, HCDR2, and HCDR3 of SEQ ID NOs: 53, 54, and 55, respectively. In some embodiments, an anti-CD3 LCVR comprises the LCDR1, LCDR2, and LCDR3 of SEQ ID NOs: 50, 51, and 52, respectively.

In one embodiment, the present disclosure provides a nucleic acid molecule or nucleic acid molecules that comprise a nucleotide sequence encoding the HCVR sequence of the anti-CD20 antigen-binding domain comprising SEQ ID NO: 44, a nucleotide sequence encoding the HCVR sequence of the anti-CD3 antigen-binding domain comprising SEQ ID NO: 46, and a nucleotide sequence encoding the LCVR sequence comprising SEQ ID NO: 45.

In another aspect, the present disclosure also provides recombinant expression vectors carrying one or more nucleic acid molecules as disclosed herein, as well as host cells into which such vectors have been introduced. In some embodiments, the host cell is a prokaryotic cell (e.g., E. coli). In some embodiments, the host cell is a eukaryotic cell, such as a non-human mammalian cell (e.g., a Chinese Hamster Ovary (CHO) cell). Also provided herein are methods of producing the antigen-binding molecules of the disclosure by culturing the host cells under conditions permitting production of the antigen-binding molecules, and recovering the antigen-binding molecules so produced.

### VI. Characterization of BCMA×CD3 Bispecific Antigen-Binding Molecules and CD20×CD3 Bispecific Antigen-Binding Molecules

The present disclosure includes bispecific antigen-binding molecules (e.g., bispecific antibodies) and functional fragments thereof that bind to BCMA and CD3 (e.g., human BCMA and CD3) with high affinity.

In some embodiments, the present disclosure includes bispecific antigen-binding molecules (e.g., bispecific antibodies as disclosed herein) that bind BCMA and CD3 (e.g., at 25° C. or 37° C.) with a K_{D} of less than about 75 nM, e.g., as measured by surface plasmon resonance or a substantially similar assay. In certain embodiments, the antigen-binding molecules of the present disclosure bind human BCMA and CD3 with a K_{D} of less than about 75 nM, less than about 70 nM, less than about 60 nM, less than about 50 nM, less than about 40 nM, less than about 30 nM, less than about 25 nM, less than about 20 nM, less than about 15 nM, less than about 10 nM, less than about 5 nM, less than about 1 nM, less than about 500 pM, less than about 400 pM, less than about 300 pM, less than about 200 pM, less than about 100 pM, less than about 90 pM, less than about 80 pM, less than about 70 pM, less than about 60 pM, less than about 50 pM, less than about 40 pM, less than about 30 pM, less than about 20 pM, less than about 10 pM, less than about 5 pM, less than about 4 pM, less than about 2 pM, less than about 1 pM, less than about 0.5 pM, less than about 0.2 pM, less than about 0.1 pM, or less than about 0.05 pM, as measured by surface plasmon resonance or a substantially similar assay.

In some embodiments, the present disclosure includes bispecific antigen-binding molecules (e.g., bispecific antibodies as disclosed herein) that specifically interact (e.g., bind with) cells that express BCMA and/or CD3. The extent to which an antigen-binding molecule binds cells that express BCMA and/or CD3 can be assessed by flow cytometry. For example, in some embodiments, the present disclosure provides anti-BCMA×CD3 bispecific antibodies that specifically bind cells that express BCMA and/or CD3 on the cell surface (e.g., human plasma cells and/or T cells). In some embodiments, the disclosure provides anti-BCMA×CD3 bispecific antibodies that bind BCMA and/or CD3-expressing cells or cell lines with an EC₅₀ value of about 10 nM or less, e.g., from about 0.5 nM to about 10 nM, e.g., an EC₅₀ value of about 1 nM, about 1.5 nM, about 2 nM, about 2.5 nM, about 3 nM, about 3.5 nM, about 4 nM, about 4.5 nM, about 5 nM, about 5.5 nM, about 6 nM, about 6.5 nM, about 7 nM, about 7.5 nM, about 8 nM, about 8.5 nM, about 9 nM, about 9.5 nM, or about 10 nM, e.g., as determined by flow cytometry or a substantially similar assay.

The present disclosure includes bispecific antigen-binding molecules (e.g., bispecific antibodies) and functional fragments thereof that bind to CD20 and CD3 (e.g., human CD20 and CD3) with high affinity.

In some embodiments, the present disclosure includes bispecific antigen-binding molecules (e.g., bispecific antibodies as disclosed herein) that specifically interact (e.g., bind with) cells that express CD20 and/or CD3. The extent to which an antigen-binding molecule binds cells that express CD20 and/or CD3 can be assessed by an in vitro binding assay. For example, in some embodiments, the present disclosure provides anti-CD20×CD3 bispecific antibodies that specifically bind cells that express CD20 and/or CD3 on the cell surface (e.g., human B cells and/or T cells). In certain embodiments, the anti-CD20×CD3 bispecific antibodies bind Jurkat cells and Raji cells with an EC₅₀ value of less than about 60 nM, as measured by an in vitro binding assay. In certain embodiments, the anti-CD20×CD3 bispecific antibodies bind CD3 or CD20 on the surface of a Jurkat or Raji cell, respectively, with an EC₅₀ value of less than about 1000 mM, less than about 500 nM, less than about 200 nM, less than about 100 nM, less than about 75 nM, less than about 70 nM, less than about 65 nM, less than about 60 nM, less than about 50 nM, less than about 40 nM, less than about 30 nM, less than about 25 nM, less than about 10 nM, less than about 5 nM, less than about 2 nM, less than about 1 nM, less than about 500 pM, less than about 100 pM, less than about 10 pM, or less than about 1 pM as measured by an in vitro binding assay.

### VIII. Epitope Mapping and Related Technologies

In some embodiments, the epitope on BCMA and/or CD20 and/or CD3 to which the antigen-binding molecules of the present disclosure bind (e.g., an epitope of BCMA or CD20 to which a first antigen-binding domain (D1) binds, or an epitope of CD3 to which a second antigen-binding domain (D2) binds) may consist of a single contiguous sequence of 3 or more (e.g., 3, 4, 5, 6, 7, 8, 9, 10, 11, 12, 13, 14, 15, 16, 17, 18, 19, 20 or more) amino acids of a BCMA or CD20 or CD3 protein. Alternatively, the epitope may consist of a plurality of non-contiguous amino acids (or amino acid sequences) of a BCMA or CD20 or CD3 protein. The antibodies of the invention may interact with amino acids contained within a single CD3 chain (e.g., CD3-epsilon, CD3-delta or CD3-gamma), or may interact with amino acids on two or more different CD3 chains. The term “epitope,” as used herein, refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

Various techniques known to persons of ordinary skill in the art can be used to determine whether an antigen-binding domain of an antibody “interacts with one or more amino acids” within a polypeptide or protein. Exemplary techniques that can be used to determine an epitope or binding domain of a particular antibody or antigen-binding domain include, e.g., routine crossblocking assay such as that described in Antibodies, Harlow and Lane (Cold Spring Harbor Press, Cold Spring Harb., NY), point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystal structure analysis can also be used to identify the amino acids within a polypeptide with which an antibody interacts.

The present disclosure also includes antigen-binding molecules (e.g., antibodies or antigen-binding domains thereof) that bind to the same epitope as, or competes for binding with, a bispecific BCMA×CD3 antigen-binding molecule or a bispecific CD20×CD3 antigen-binding molecule described herein. One skilled in the art can determine whether or not a particular antigen-binding molecule (e.g., antibody) or antigen-binding domain thereof binds to the same epitope as, or competes for binding with, a reference antigen-binding molecule of the present disclosure by using routine methods known in the art. For example, to determine if a test antibody binds to the same epitope on BCMA and/or CD20 and/or CD3 as a reference bispecific antigen-binding molecule of the present disclosure, the reference bispecific molecule is first allowed to bind to a BCMA and/or CD20 and/or CD3 protein. Next, the ability of a test antibody to bind to the BCMA and/or CD20 and/or CD3 molecule is assessed. If the test antibody is able to bind to BCMA and/or CD20 and/or CD3 following saturation binding with the reference bispecific antigen-binding molecule, it can be concluded that the test antibody binds to a different epitope of BCMA and/or CD20 and/or CD3 than the reference bispecific antigen-binding molecule. On the other hand, if the test antibody is not able to bind to the BCMA and/or CD20 and/or CD3 molecule following saturation binding with the reference bispecific antigen-binding molecule, then the test antibody may bind to the same epitope of BCMA and/or CD20 and/or CD3 as the epitope bound by the reference bispecific antigen-binding molecule of the disclosure. Additional routine experimentation (e.g., peptide mutation and binding analyses) can then be carried out to confirm whether the observed lack of binding of the test antibody is in fact due to binding to the same epitope as the reference bispecific antigen-binding molecule or if steric blocking (or another phenomenon) is responsible for the lack of observed binding. Experiments of this sort can be performed using ELISA, radioimmunoassay (RIA), Biacore, flow cytometry or any other quantitative or qualitative antibody-binding assay available in the art. In accordance with certain embodiments of the present disclosure, two antigen-binding proteins bind to the same (or overlapping) epitope if, e.g., a 1-, 2-, 5-, 10-, 20- or 100-fold excess of one antigen-binding protein inhibits binding of the other by at least 50% but preferably 75%, 90% or even 99% as measured in a competitive binding assay (see, e.g., Junghans et al., Cancer Res. 1990:50:1495-1502). Alternatively, two antigen-binding proteins are deemed to bind to the same epitope if essentially all amino acid mutations in the antigen that reduce or eliminate binding of one antigen-binding protein reduce or eliminate binding of the other. Two antigen-binding proteins are deemed to have “overlapping epitopes” if only a subset of the amino acid mutations that reduce or eliminate binding of one antigen-binding protein reduce or eliminate binding of the other.

To determine if an antibody or antigen-binding domain thereof competes for binding with a reference antigen-binding molecule, the above-described binding methodology is performed in two orientations: In a first orientation, the reference antigen-binding molecule is allowed to bind to a BCMA and/or CD20 and/or CD3 protein under saturating conditions followed by assessment of binding of the test antibody to the BCMA and/or CD20 and/or CD3 molecule. In a second orientation, the test antibody is allowed to bind to a BCMA and/or CD20 and/or CD3 molecule under saturating conditions followed by assessment of binding of the reference antigen-binding molecule to the BCMA and/or CD20 and/or CD3 molecule. If, in both orientations, only the first (saturating) antigen-binding molecule is capable of binding to the BCMA and/or CD20 and/or CD3 molecule, then it is concluded that the test antibody and the reference antigen-binding molecule compete for binding to BCMA and/or CD20 and/or CD3. As will be appreciated by a person of ordinary skill in the art, an antibody that competes for binding with a reference antigen-binding molecule may not necessarily bind to the same epitope as the reference antibody, but may sterically block binding of the reference antibody by binding an overlapping or adjacent epitope.

### IX. Preparation of Antigen-Binding Domains and Constructions of Multispecific Antigen-Binding Molecules

Antigen-binding domains specific for particular antigens can be prepared by any antibody generating technology known in the art. Once obtained, two different antigen-binding domains can be appropriately arranged relative to one another to produce a bispecific antigen-binding molecule of the present disclosure using routine methods. A discussion of exemplary bispecific antibody formats that can be used to construct the bispecific antigen-binding molecules of the present disclosure is provided elsewhere herein. In certain embodiments, one or more of the individual components (e.g., heavy, and light chains) of the multispecific antigen-binding molecules are derived from chimeric, humanized or fully human antibodies. Methods for making such antibodies are well known in the art. For example, one or more of the heavy and/or light chains of the bispecific antigen-binding molecules of the present disclosure can be prepared using VELOCIMMUNE™ technology. Using VELOCIMMUNE™ technology (or any other human antibody generating technology), high affinity chimeric antibodies to a particular antigen (e.g., BCMA or CD20 or CD3) are initially isolated having a human variable region and a mouse constant region. The antibodies are characterized and selected for desirable characteristics, including affinity, selectivity, epitope, etc. The mouse constant regions are replaced with a desired human constant region to generate fully human heavy and/or light chains that can be incorporated into the bispecific antigen-binding molecules.

In some embodiments, genetically engineered animals may be used to make human bispecific antigen binding molecules. For example, a genetically modified mouse can be used which is incapable of rearranging and expressing an endogenous mouse immunoglobulin light chain variable sequence, wherein the mouse expresses only one or two human light chain variable domains encoded by human immunoglobulin sequences operably linked to the mouse kappa constant gene at the endogenous mouse kappa locus. Such genetically modified mice can be used to produce fully human bispecific antigen-binding molecules comprising two different heavy chains that associate with an identical light chain that comprises a variable domain derived from one of two different human light chain variable region gene segments. See, e.g., US 2011/0195454, the entire contents of which are incorporated herein by reference, for a detailed discussion of such engineered mice and the use thereof to produce bispecific antigen-binding molecules. As used herein, “fully human” refers to an antigen-binding molecule, e.g., an antibody, or antigen-binding fragment or immunoglobulin domain thereof, comprising an amino acid sequence encoded by a DNA derived from a human sequence over the entire length of each polypeptide of the antigen-binding molecule, antibody, antigen-binding fragment, or immunoglobulin domain thereof. In some instances, the fully human sequence is derived from a protein endogenous to a human. In other instances, the fully human protein or protein sequence comprises a chimeric sequence wherein each component sequence is derived from human sequence. While not being bound by any one theory, chimeric proteins or chimeric sequences are generally designed to minimize the creation of immunogenic epitopes in the junctions of component sequences, e.g., compared to any wild-type human immunoglobulin regions or domains.

### X. Bioequivalents

The present disclosure encompasses antigen-binding molecules having amino acid sequences that vary from those of the described antibodies but that retain the ability to bind BCMA and/or CD20 and/or CD3. Such variant molecules comprise one or more additions, deletions, or substitutions of amino acids when compared to the parent sequence, but exhibit biological activity that is essentially equivalent to that of the described antigen-binding molecules. Likewise, the nucleic acid sequences encoding the antigen-binding molecules of the present disclosure encompass sequences that comprise one or more additions, deletions, or substitutions of nucleotides when compared to the disclosed sequence, but that encode an antigen binding molecule that is essentially bioequivalent to the antigen-binding molecules disclosed herein.

The present disclosure includes antigen-binding molecules that are bioequivalent to any of the exemplary antigen-binding molecules set forth herein. Two antigen-binding proteins, e.g., bispecific antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the first antigen-binding protein (e.g., reference product) and the second antigen-binding protein (e.g., biological product) without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Non-limiting examples of bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the exemplary bispecific antigen-binding molecules set forth herein may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other embodiments, bioequivalent antibodies may include the exemplary bispecific antigen-binding molecules set forth herein comprising amino acid changes which modify the glycosylation characteristics of the antibodies, e.g., mutations which eliminate or remove glycosylation.

### XI. Immunoglobulin Depleting Agents

In various aspects, the present disclosure provides immunoglobulin depleting agents, e.g., which may be combined with or administered in combination with plasma cell depleting agents (e.g., an anti-BCMA×CD3 bispecific antibody, or a functional fragment thereof or B cell depleting agents (e.g., an anti-CD20×CD3 bispecific antibody, or a functional fragment thereof) described herein. In some embodiments, the immunoglobulin depleting agent may be administered in combination with a plasma cell depleting agent, a B cell depleting agent, plasmapheresis, therapeutic plasma exchange, immunoadsorption, and/or an immunogen (e.g., nucleic acid construct, nuclease agent or CRISPR/Cas system, e.g., in an immunogenic delivery vehicle) (e.g., an immunogenic delivery vehicle such as, e.g., AAV) disclosed herein. Suitable combinations comprising a plasma cell depleting agent are described in more detail elsewhere herein. In some embodiments, an immunoglobulin depleting agent may be useful for, e.g., accelerating IgG clearance.

In some embodiments, an immunoglobulin depleting agent is capable of accelerating IgG serum clearance.

In some embodiments, an immunoglobulin depleting agent may comprise a neonatal Fc receptor (FcRn) blocker such as, but not limited to, efgartigimod alfa. The mechanistic concept of FcRn-targeting therapeutics is to accelerate IgG catabolism by blocking the FcRn-mediated intracellular IgG recycling pathway, thereby reducing overall plasma IgG levels. FcRn can participate in the maintenance of IgG levels by salvaging IgG from lysosomal degradation, thereby prolonging the half-life of IgG. In some embodiments, FcRn blockers can compete with IgG for binding to FcRn. Due to their higher affinity for FcRn, FcRn blockers can prevent IgG from binding to FcRn and, instead, IgG is transported to the lysosome and degraded, thereby leading to decreased circulating levels of IgG.

In some embodiments, an FcRn blocker can include Efgartigimod (ARGX-113), Rozanolixizumab (UCB7665), Batoclimab (RVT-1401), IMVT-1402, Nipocalimab (M281), Orilanolimab (SYNT01), or any combination thereof. See, e.g., Zuercher et al. (2019) Autoimmun. Rev. 18(10):102366.

In some embodiments, an immunoglobulin depleting agent may comprise an IgG degrading enzyme such as IdeS (imlifidase), IdeZ, or IdeXork. IdeS (imlifidase) is an endopeptidase derived from Streptococcus pyogenes which has specificity for human IgG, and when infused intravenously results in rapid cleavage of IgG. IdeZ (immunoglobulin-degrading enzyme from Streptococcus equi subspecies zooepidemicus) is an engineered recombinant protease overexpressed in Escherichia coli. IdeZ specifically cleaves IgG molecules below the hinge region to yield F(ab′)2 and Fc fragments. IdeXork (Xork) is yet another example of an IgG protease. Additional non-limiting examples of IgG degrading enzymes include Imlifidase/IdeS/Fabricator, IdeZ, IceM, IceMG, CYR-212, CYR-241, S-1117, HNSA-5487, and Xork. In some embodiments, an immunoglobulin depleting agent may facilitate IgG degradation via lysosomal destruction. A non-limiting example of an immunoglobulin depleting agent which may facilitate IgG degradation via lysosomal destruction is BHV-1300.

### XII. Plasmapheresis, Therapeutic Plasma Exchange, and Immunoadsorption

In various aspects, the methods disclosed herein can include plasmapheresis, therapeutic plasma exchange, or immunoadsorption. These can be combined, for example, with treatment with plasma cell depleting agents (e.g., an anti-BCMA×CD3 bispecific antibody, or a functional fragment thereof), B cell depleting agents (e.g., an anti-CD20×CD3 bispecific antibody, or a functional fragment thereof), and/or immunoglobulin depleting agents described herein. In some embodiments, the plasmapheresis, therapeutic plasma exchange, or immunoadsorption may be performed in combination with treatment with a plasma cell depleting agent, a B cell depleting agent, and/or an immunogen (e.g., nucleic acid construct, nuclease agent or CRISPR/Cas system, e.g., in an immunogenic delivery vehicle) disclosed herein. Suitable combinations comprising a plasma cell depleting agent are described in more detail elsewhere herein. Plasmapheresis, therapeutic plasma exchange, and immunoadsorption may be useful strategies for removal of AAV antibodies from patients' blood plasma.

Plasmapheresis is a process used to selectively remove blood components used to treat a variety of conditions including those caused by the acute overproduction of antibodies (e.g., autoimmunity, transplant rejection), in which removal of pathogenic immunoglobulins results in clinical benefit. Immunoadsorption is a selective therapeutic apheresis technique by which immunoglobulins are selectively removed from patients' plasma. The immunoadsorption can be, for example, total immunoglobulin immunoadsorption. See, e.g., Boedecker-Lips et al. (2023) J. Clin. Apher. 38(5):590-601. Alternatively, the immunoadsorption can be AAV capsid specific immunoadsorption. See, e.g., Bertin et al. (2020) Sci. Rep. 10:864.

### XIII. Combinations Comprising a Plasma Cell Depleting Agent

A plasma cell depleting agent (e.g., a BCMA×CD3 antigen-binding molecule) can be administered to a subject in need thereof either alone, or in combination with, a B cell depleting agent (e.g., a CD20×CD3 antigen-binding molecule), an immunoglobulin depleting agent (e.g., an FcRn blocker, such as Efgartigimod), and/or an immunogen. In some embodiments, the administration of the plasma cell depleting, the B cell depleting agent, the immunoglobulin depleting agent, and/or the immunogen can be further combined with plasmapheresis, therapeutic plasma exchange, and/or immunoadsorption. As used herein, the term “in combination with,” e.g., a BCMA×CD3 bispecific antigen-binding molecule (or other immunomodulator or immunogen, etc.) means that additional component(s) may be administered prior to, concurrent with, or after the administration of BCMA×CD3 bispecific antigen-binding molecule (or other immunomodulator or immunogen, etc.) molecule (or other immunomodulator or immunogen, etc.). The different components of the combination can be formulated into a single composition, e.g., for simultaneous delivery, or formulated separately into two or more compositions (e.g., a kit including each component, for example, wherein the further agent is in a separate formulation).

For example, a plasma cell depleting agent (e.g., a BCMA×CD3 antigen-binding molecule) can be administered to a subject in need thereof either alone, or in combination with, a B cell depleting agent (e.g., a CD20×CD3 antigen-binding molecule) and/or an immunoglobulin depleting agent (e.g., an FcRn blocker, such as Efgartigimod). In some embodiments, the B cell depleting agent is administered before, at the same time as, or after the plasma cell depleting agent. In some embodiments, the immunoglobulin depleting agent is administered after the plasma cell depleting agent. In some embodiments, the B cell depleting agent is administered prior to and after the nucleic acid construct. In some embodiments, the immunoglobulin depleting agent is administered prior to and after the nucleic acid construct. In some embodiments, the immunoglobulin depleting agent is administered after an initial dose of the plasma cell depleting agent, or wherein the immunoglobulin depleting agent is administered after an initial dose of the plasma cell depleting agent and after an initial dose of the B cell depleting agent.

In one example, a plasma cell depleting agent (e.g., a BCMA×CD3 antigen-binding molecule) is administered to a subject in need thereof in combination with a B cell depleting agent (e.g., a CD20×CD3 antigen-binding molecule).

In another example, a plasma cell depleting agent (e.g., a BCMA×CD3 antigen-binding molecule) is administered to a subject in need thereof in combination with an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In some embodiments, the combination of the plasma cell depleting agent and the immunoglobulin depleting agent, when administered in further combination with an immunogen (e.g., an immunogenic delivery vehicle such as, e.g., AAV) to a subject in need thereof, decreases a level of an anti-immunogen antibody titer (e.g., an anti-AAV antibody titer) in the subject (e.g., such as can be measured in a serum sample isolated from the subject). In some embodiments, the level of the anti-immunogen antibody titer is decreased by about 1-fold to about 20-fold, about 2-fold to about 15-fold, about 4-fold to about 10-fold, about 3-fold to about 18-fold, about 5-fold to about 12-fold, or about 6-fold to about 8-fold, as compared to the level of the anti-immunogen antibody titer in a subject administered the immunogen alone. In some embodiments, the anti-immunogen antibody titer is decreased by about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, or about 20-fold, or more. In some embodiments, the anti-immunogen antibody titer is decreased by about 20-fold.

In another example, a plasma cell depleting agent (e.g., a BCMA×CD3 antigen-binding molecule) is administered to a subject in need thereof in combination with a B cell depleting agent (e.g., a CD20×CD3 antigen-binding molecule) and an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In some embodiments, the combination of the plasma cell-depleting agent, the B cell depleting agent, and the immunoglobulin-depleting agent, when administered in further combination with an immunogen (e.g., an immunogenic delivery vehicle such as, e.g., AAV) to a subject in need thereof, decreases the level of an anti-immunogen antibody titer (e.g., an anti-AAV antibody titer) in the subject (e.g., such as can be measured in a serum sample isolated from the subject). In some embodiments, the level of the anti-immunogen antibody titer may be decreased by about 1-fold to about 20-fold, about 2-fold to about 15-fold, about 4-fold to about 10-fold, about 3-fold to about 18-fold, about 5-fold to about 12-fold, about 6-fold to about 8-fold, about 10-fold to about 30-fold, about 20-fold to about 50-fold, about 30-fold to about 70-fold, about 40-fold to about 90-fold, or about 50-fold to about 100-fold, as compared to the level of the anti-immunogen antibody titer in a subject administered the immunogen alone. In some embodiments, the anti-immunogen antibody titer is decreased by about 1-fold, about 2-fold, about 3-fold, about 4-fold, about 5-fold, about 6-fold, about 7-fold, about 8-fold, about 9-fold, about 10-fold, about 11-fold, about 12-fold, about 13-fold, about 14-fold, about 15-fold, about 16-fold, about 17-fold, about 18-fold, about 19-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, or about 100-fold, or more. In some embodiments, the anti-immunogen antibody titer is decreased by about 100-fold.

In another example, a plasma cell depleting agent (e.g., a BCMA×CD3 antigen-binding molecule) is administered to a subject in need thereof in combination with plasmapheresis, therapeutic plasma exchange, or immunoadsorption.

In another example, a plasma cell depleting agent (e.g., a BCMA×CD3 antigen-binding molecule) is administered to a subject in need thereof in combination with plasmapheresis, therapeutic plasma exchange, or immunoadsorption and a B cell depleting agent (e.g., a CD20×CD3 antigen-binding molecule).

In another example, a plasma cell depleting agent (e.g., a BCMA×CD3 antigen-binding molecule) is administered to a subject in need thereof in combination with plasmapheresis, therapeutic plasma exchange, or immunoadsorption and an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In another example, a plasma cell depleting agent (e.g., a BCMA×CD3 antigen-binding molecule) is administered to a subject in need thereof in combination with plasmapheresis, therapeutic plasma exchange, or immunoadsorption, a B cell depleting agent (e.g., a CD20×CD3 antigen-binding molecule), and an immunoglobulin depleting agent (e.g., an FcRn blocker, such as Efgartigimod). In some embodiments, the immunoglobulin depleting agent comprises an FcRn blocker. In some embodiments, the immunoglobulin depleting agent comprises an IgG degrading enzyme.

In some embodiments, the B cell depleting agent comprises two or more B cell depleting agents (e.g., an anti-CD19 antigen-binding molecule and an anti-CD20 antigen-binding molecule). In some embodiments, the immunoglobulin depleting agent comprises two or more immunoglobulin depleting agents (e.g., an FcRn blocker and an IgG degrading enzyme).

In embodiments in which a plasma cell depleting agent (e.g., a BCMA×CD3 antigen-binding molecule) is administered to a subject in combination with, a B cell depleting agent (e.g., a CD20×CD3 antigen-binding molecule) and/or an immunoglobulin depleting agent (e.g., an FcRn blocker, such as Efgartigimod) and/or plasmapheresis, therapeutic plasma exchange, or immunoadsorption, one or more or all treatments can occur together or one or more or all treatments can occur sequentially. For example, in some embodiments in which the plasma cell depleting agent (e.g., a BCMA×CD3 antigen-binding molecule) is administered to a subject in need thereof in combination with an IgG degrading enzyme, the plasma cell depleting agent can be administered to the subject first, followed by the IgG degrading enzyme. In another example, in embodiments where an immunoglobulin depleting agent (e.g., FcRn blocker) is administered together with plasmapheresis, therapeutic plasma exchange, or immunoadsorption, the plasmapheresis, therapeutic plasma exchange, or immunoadsorption can be first followed by administration of the immunoglobulin depleting agent (e.g., FcRn blocker).

### XIV. Pharmaceutical Compositions

In another aspect, the present disclosure provides pharmaceutical compositions comprising plasma cell depleting agents (e.g., long-lived plasma cell (LLPC) depleting agents such as anti-BCMA×CD3 bispecific antibodies, or functional fragments thereof), B cell depleting agents (e.g., anti-CD19 and anti-CD20 antibodies or a CD20×CD3 antigen-binding molecule (e.g., REGN1979), or functional fragments thereof), immunoglobulin depleting agents (e.g., neonatal Fc receptor (FcRn) blockers), and/or immunogens (e.g., nucleic acid construct, nuclease agent or CRISPR/Cas system, e.g., in an immunogenic delivery vehicle) (e.g., immunogenic delivery vehicles) disclosed herein, optionally comprising a pharmaceutically acceptable carrier and/or excipient. In one specific embodiment, a composition described herein comprises an immunogen and an anti-CD20×CD3 bispecific antibody, or a functional fragment thereof, and optionally, further comprises a pharmaceutically acceptable carrier and/or excipient. Suitable combinations comprising a plasma cell depleting agent are described in more detail elsewhere herein. The pharmaceutical compositions are formulated with one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA.

One exemplary embodiment of the present disclosure comprises a pharmaceutical composition comprising (i) a plasma cell depleting agent, (ii) a B cell depleting agent and/or an immunoglobulin depleting agent, and (iii) a pharmaceutically acceptable carrier and/or excipient. Another exemplary embodiment of the present disclosure comprises a pharmaceutical composition comprising (i) an immunogen, (ii) a plasma cell depleting agent, (iii) optionally, a B cell depleting agent and/or an immunoglobulin depleting agent, and (iv) a pharmaceutically acceptable carrier and/or excipient.

In some embodiments, the plasma cell depleting agent comprises an antigen-binding molecule that specifically binds B cell maturation antigen (BCMA) and CD3. In some embodiments, the plasma cell depleting agent comprises an anti-BCMA×CD3 bispecific antibody, or functional fragment thereof, disclosed herein. Non-limiting examples of an anti-BCMA×CD3 bispecific antibody include linvoseltamab (REGN5458), REGN5459, pacanalotamab (AMG420), teclistamab (JNJ-64007957), AMG701, alnuctamab (CC-93269), EM801, EM901, elranatamab (PF-06863135), TNB383B (ABBV-383), and TNB384B. In a specific embodiment, the anti-BCMA×CD3 bispecific antibody is REGN5458. In another specific embodiment, the anti-BCMA×CD3 bispecific antibody is REGN5459.

In some embodiments, the anti-BCMA×CD3 bispecific antibody comprises: (a) a first antigen-binding domain (D1) that binds an epitope of human BCMA; and (b) a second antigen-binding domain (D2) that binds an epitope of human CD3.

In some embodiments, the B cell depleting agent comprises anti-CD19 and anti-CD20 antibodies, or functional fragments thereof, disclosed herein. In some embodiments, the B cell depleting agent comprises a CD20×CD3 antigen-binding molecule (e.g., REGN1979).

In some embodiments, the immunoglobulin depleting agent comprises a neonatal Fc receptor (FcRn) blocker. A non-limiting example of an FcRn blocker is efgartigimod alfa. In some embodiments, the immunoglobulin depleting agent comprises an IgG degrading enzyme.

In some embodiments, the immunogen is an immunogenic delivery vehicle, a polypeptide, or a polynucleotide. In some embodiments, the immunogen is an immunogenic delivery vehicle or a polypeptide or polynucleotide encoded by a transgene contained within the immunogenic delivery vehicle.

In some embodiments, the immunogen is an immunogenic delivery vehicle and/or transgene product(s).

In some embodiments, the immunogenic delivery vehicle is a viral vector, a virus-like particle (VLP), a lipid nanoparticle (LNP), a non-lipid nanoparticle, a liposome, a bacterial vector, a fungal vector, a protozoal vector, or a mammalian cell.

In some embodiments, the immunogenic delivery vehicle is a viral vector.

In some embodiments, the viral vector is derived from an adeno-associated virus (AAV), an adenovirus, a retrovirus, or an oncolytic virus.

In some embodiments, the viral vector is AAV. In some embodiments, the viral vector is recombinant AAV. In some embodiments, the viral vector is derived from AAV.

In some embodiments, the retrovirus is a lentivirus.

In some embodiments, the oncolytic virus is an adenovirus, a rhabdovirus, a herpes virus, a measles virus, a coxsackievirus, a poliovirus, a reovirus, a poxvirus, a parvovirus, Maraba virus, or Newcastle disease virus.

In some embodiments, the carrier is suitable for intravenous, intramuscular, oral, intraperitoneal, intratumoral, intrathecal, transdermal, topical, or subcutaneous administration.

In some embodiments, the pharmaceutical composition comprises an injectable preparation, such as a dosage form for intravenous, subcutaneous, intracutaneous, and intramuscular injections, drip infusions, etc. These injectable preparations may be prepared by known methods. For example, the injectable preparations may be prepared, e.g., by dissolving, suspending, or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle) administered to a patient according to the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions as disclosed herein may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

In some embodiments, e.g., for methods and compositions of the present disclosure involving administration of a plasma cell depleting agent which is a bispecific BCMA×CD3 antibody (e.g., REGN5458) to a subject, the dose of the bispecific BCMA×CD3 antibody (or pharmaceutical compositions thereof) is from about 1 mg/kg to about 30 mg/kg, such as from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg. In some embodiments, the bispecific BCMA×CD3 antibody (e.g., REGN5458) can be administered to the subject at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In one specific embodiment, the bispecific BCMA×CD3 antibody (e.g., REGN5458) (or pharmaceutical composition thereof) dose is about 20 mg/kg.

In some embodiments, e.g., for methods and compositions of the present disclosure involving administration of a B cell depleting agent which is a bispecific CD20×CD3 antibody (e.g., REGN1979) to a subject, the dose of the bispecific CD20×CD3 antibody (or pharmaceutical compositions thereof) is from about 0.05 mg/kg to about 3 mg/kg, such as from about 0.05 mg/kg to about 0.1 mg/kg, about 0.1 mg/kg to about 0.5 mg/kg, about 0.5 mg/kg to about 1 mg/kg, about 1 mg/kg to about 1.5 mg/kg, about 1.5 mg/kg to about 2 mg/kg, about 2 mg/kg to about 2.5 mg/kg, or about 2.5 mg/kg to about 3 mg/kg. In one specific embodiment, the bispecific CD20×CD3 antibody (e.g., REGN1979) is administered to the subject at a dose of about 0.1 mg/kg. In another specific embodiment, the bispecific CD20×CD3 antibody (e.g., REGN1979) is administered to the subject at a dose of about 1 mg/kg.

In some embodiments, e.g., for methods and compositions of the present disclosure involving administration of an immunoglobulin depleting agent which is efgartigimod to a subject, the dose of efgartigimod (or pharmaceutical compositions thereof) is from about 1 mg/kg to about 30 mg/kg, such as from about 1 mg/kg to about 5 mg/kg, about 5 mg/kg to about 10 mg/kg, about 10 mg/kg to about 15 mg/kg, about 15 mg/kg to about 20 mg/kg, about 20 mg/kg to about 25 mg/kg, or about 25 mg/kg to about 30 mg/kg. In some embodiments, efgartigimod can be administered to the subject at a dose of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, or about 30 mg/kg. In one specific embodiment, the efgartigimod (or pharmaceutical composition thereof) dose is about 20 mg/kg.

In some embodiments, e.g., for methods and compositions of the present disclosure involving administration of an immunogen which is an AAV to a subject, the dose of the AAV (or pharmaceutical compositions thereof) administered to a subject is between about 1×10⁵ plaque forming units (pfu) to about 1×10¹⁵ pfu. In some cases, the AAV can be administered to the subject at a dose from about 1×10⁸ pfu to about 1×10¹⁵ pfu, or from about 1×10¹⁰ pfu to about 1×10¹⁵ pfu, or from about 1×10⁸ pfu to about 1×10¹² pfu.

In some embodiments, the dose of the AAV (or pharmaceutical compositions thereof) administered to the subject is between about 1×10⁵ vg to about 1×10¹⁶ vg. In certain embodiments, the dose of the AAV administered to the subject is between about 1×10⁶ vg to about 1×10⁹ vg, about 1×10⁷ vg to about 1×10¹⁰ vg, about 1×10⁸ vg to about 1×10¹¹ vg, about 1×10⁹ vg to about 1×10¹² vg, about 1×10¹⁰ vg to about 1×10¹³ vg, about 1×10¹¹ vg to about 1×10¹⁴ vg, about 1×10¹² vg to about 1×10¹⁵ vg, about 1×10¹³ vg to about 1×10¹⁶ vg, or about 1×10¹⁴ vg to about 1×10¹⁶ vg. In certain embodiments, the dose of the AAV administered to the subject is between about 1×10¹⁰ vg to about 1×10¹⁶ vg. In certain embodiments, the dose of the AAV administered to the subject is at least about 1×10⁶ vg, at least about 1×10⁷ vg, at least about 1×10⁸ vg, at least about 1×10⁹ vg, at least about 1×10¹⁰ vg, at least about 1×10¹¹ vg, at least about 1×10¹² vg, at least about 1×10¹² vg, at least about 1×10¹³ vg, at least about 1×10¹⁴ vg, or at least about 1×10¹⁵ vg. In certain embodiments, the vg is total vector genome per subject.

In some embodiments, the dose of the AAV (or pharmaceutical compositions thereof) administered to the subject is about 1×10¹², 1×10¹³, 1×10¹⁴, 1×10¹⁵, and 1×10¹⁶ vector genomes (vg)/mL. Further examples of doses of AAV include about 1×10¹², about 1×10¹³, about 1×10¹⁴, about 1×10¹⁵, and about 1×10¹⁶ vector genomes (vg)/mL, or between about 1×10¹² to about 1×10¹⁶, between about 1×10¹² to about 1×10¹⁵, between about 1×10¹² to about 1×10¹⁴, between about 1×10¹² to about 1×10¹³, between about 1×10¹³ to about 1×10¹⁶, between about 1×10¹⁴ to about 1×10¹⁶, between about 1×10¹⁵ to about 1×10¹⁶, or between about 1×10¹³ to about 1×10¹⁵ vg/mL.

Other examples of doses of AAV (or pharmaceutical compositions thereof) include about 1×10¹², about 1×10¹³, about 1×10¹⁴, about 1×10¹⁵, and about 1×10¹⁶ vector genomes (vg)/kg of body weight, or between about 1×10¹² to about 1×10¹⁶, between about 1×10¹² to about 1×10¹⁵, between about 1×10¹² to about 1×10¹⁴, between about 1×10¹² to about 1×10¹³, between about 1×10¹³ to about 1×10¹⁶, between about 1×10¹⁴ to about 1×10¹⁶, between about 1×10¹⁵ to about 1×10¹⁶, or between about 1×10¹³ to about 1×10¹⁵ vg/kg of body weight.

In one example, the AAV dose (or pharmaceutical compositions thereof) is between about 1×10¹³ to about 1×10¹⁴ vg/mL or vg/kg. In another example, the AAV dose is between about 1×10¹² to about 1×10¹³ vg/mL or vg/kg (e.g., between about 1×10¹² to about 1×10¹³ vg/kg). In another example, the AAV dose is between about 1×10¹² to about 1×10¹⁴ vg/mL or vg/kg (e.g., between about 1×10¹² to about 1×10¹⁴ vg/kg).

In one specific embodiment, the AAV dose (or pharmaceutical composition thereof) is about 3×10¹¹ vg/kg. In one specific embodiment, the AAV dose (or pharmaceutical composition thereof) is about 6×10¹¹ vg/kg. In another specific embodiment, the AAV dose (or pharmaceutical composition thereof) is about 9×10¹¹ vg/kg. In another specific embodiment, the AAV dose (or pharmaceutical composition thereof) is about 3×10¹² vg/kg. In one specific embodiment, the AAV dose (or pharmaceutical composition thereof) is about 1×10¹³ vg/kg. In another specific embodiment, the AAV dose (or pharmaceutical composition thereof) is about 6×10¹³ vg/kg.

Various delivery systems are known and can be used to administer the pharmaceutical composition, e.g., encapsulation in liposomes, microparticles, microcapsules, recombinant cells capable of expressing, e.g., recombinant viruses comprising any components of the compositions disclosed herein, and a soluble carrier system that takes advantage of receptor mediated endocytosis (see, e.g., Wu et al., 1987, J. Biol. Chem. 262:4429-4432). Methods of administration include, but are not limited to, intradermal, intramuscular, intraperitoneal, intratumoral, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously. In some embodiments, a pharmaceutical composition as disclosed herein is administered intratumorally.

In some embodiments, a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle), or a pharmaceutical composition(s) thereof, is contained within a container. Thus, in another aspect, containers comprising an antigen-binding molecule and/or pharmaceutical composition as disclosed herein are provided. For example, in some embodiments, an antibody and/or pharmaceutical composition is contained within a container selected from the group consisting of a glass vial, a syringe, a pen delivery device, and an autoinjector.

In some embodiments, a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle), or a pharmaceutical composition(s) thereof, of the present disclosure is delivered, e.g., subcutaneously or intravenously, such as with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™, OPTIPEN STARLET™, and OPTICLIK™ (sanofi-aventis, Frankfurt, Germany). Examples of disposable pen delivery devices having applications, e.g., in subcutaneous delivery of a pharmaceutical composition of the present invention include, but are not limited to the SOLOSTAR™ pen (sanofi-aventis), the FLEXPEN™ (Novo Nordisk), the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park IL).

In some embodiments, the pharmaceutical compositions of the present disclosure can be delivered using a controlled release system. In one embodiment, a pump may be used (see, e.g., Langer, supra; Sefton, 1987, CRC Crit. Ref Biomed. Eng. 14:201). In another embodiment, polymeric materials can be used; see Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions as described herein are prepared into dosage forms in a unit dose suited to fit a dose of the active ingredients. Such dosage forms in a unit dose include, for example, tablets, pills, capsules, injections (ampoules), suppositories, etc. In some embodiments, the amount of the antigen-binding molecule contained in the dosage form is about 5 to about 1000 mg, e.g., from about 5 to about 500 mg, from about 5 to about 100 mg, or from about 10 to about 250 mg.

Plasma cell depleting agents, B cell depleting agents, immunoglobulin depleting agents, and/or immunogens (e.g., nucleic acid construct, nuclease agent or CRISPR/Cas system, e.g., in an immunogenic delivery vehicle), introduced into the subject or cell can be provided in compositions comprising a carrier, thereby increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of a molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection can include the use of a gene gun or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million cells as compared with 7 million cells by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids or proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors which can be useful in accomplishing virus-mediated delivery include retroviruses, adenoviruses, vaccinia viruses, poxviruses, and herpes simplex viruses. The viruses can infect dividing cells, non-dividing cells, or both dividing and non-dividing cells. The viruses can integrate into the host genome or, alternatively, do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vectors may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹³, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg).

In yet another aspect, the present disclosure includes compositions and therapeutic formulations comprising any of the plasma cell depleting agents, B cell depleting agents, immunoglobulin depleting agents, and/or immunogens (e.g., nucleic acid construct, nuclease agent or CRISPR/Cas system, e.g., in an immunogenic delivery vehicle), described herein in combination with one or more additional therapeutic agents, and methods of treatment comprising administering such combinations to subjects in need thereof. In some embodiments, the additional therapeutic agent(s) is an immunomodulatory agent or anti-inflammatory agent. In some embodiments, the additional therapeutic agent(s) is immunosuppressive therapy. In some embodiments, the additional therapeutic agent(s) is a surgical procedure.

Exemplary additional therapeutic agents that may be combined with or administered in combination with any of the plasma cell depleting agents, B cell depleting agents, immunoglobulin depleting agents, and/or immunogens (e.g., nucleic acid construct, nuclease agent or CRISPR/Cas system, e.g., in an immunogenic delivery vehicle), of the present disclosure include, e.g., an anti-CD38 antibody (e.g., daratumumab), a proteasome inhibitor, a histone deacetylase inhibitor, a B-cell activating factor (BAFF) inhibitor, an APRIL inhibitor, a steroid (e.g., corticosteroids such as topical, systemic, oral, or inhaled corticosteroids, including, but not limited to, betamethasone, clobetasol, dexamethasone, fluocinolone, fluocinonide, halobetasol, hydrocortisone, methylprednisolone, prednisone, prednisolone, or triamcinolone); a non-steroidal topical medication such as, but not limited to, a phosphodiesterase 4 (PDE4) inhibitor or a calcineurin inhibitor; a non-steroidal anti-inflammatory drug (NSAID) such as, but not limited to, celecoxib, diclofenac, etodolac, fenprofen, flurbiprofen, ibuprofen, ketoprofen, meclofamate, meloxicam, nabumetone, naproxen, oxaprozin, piroxicam, rofecoxib, salicylates, sulfasalazinem, sulindac, or tolmetin; an anti-inflammatory antibody or biologic (e.g., an anti-tumor necrosis factor alpha (anti-TNFα) antibody or biologic such as, but not limited to, adalimumab, certolizumab, etanercept, golimumab, or infliximab; an anti-IL1 antibody or biologic such as, but not limited to, LY2189102, anakinra, canakinumab, gerokizumab, or rilonacept; an anti-interleukin 6/interleukin-6 receptor (anti-IL6/IL-6R) antibody or biologic such as, but not limited to, sarilumab, siltuximab, or tocilizumab; an anti-IL17A/IL-17R antibody or biologic such as, but not limited to, bimekizumab, brodalumab, ixekizumab, or secukinumab; or an anti-IL12/IL-23 antibody or biologic such as, but not limited to, AMG139, BI655066, brazikumab, briankizumab, guselkumab, mirikizumab, risankizumab, tildrakizumab, or ustekinumab); a JAK inhibitor such as, but not limited to, abrocitinib, baricitinib, fedratinib, filgotinib, ruxolitinib, tofacitinib, or upadacitinib; an immunosuppressive agent (e.g., a systemic immunosuppressant such as but not limited to methotrexate, cyclophosphamide, mizoribine, chlorambucil, cyclosporine, mycophenolate mofetil, or azathioprine); a disease-modifying antirheumatic drug (DMARD) such as, but not limited, to apremilast, azathioprine, baricitinib, cyclophosphamide, cyclosporine, hydroxychloroquine, leflunomide, methotrexate, mycophenolate mofetil, sulfasalazine, or tofacitinib; radiation therapy; chemotherapy; intravenous immunoglobulin therapy; or a surgery or a surgical procedure (such as, but not limited to, splenectomy, lymphadenectomy, thyroidectomy, plasmapheresis, leukapheresis, therapeutic plasma exchange, immunoadsorption, or cell, tissue, or organ transplantation). In some embodiments, the surgery or surgical procedure as described herein is used in combination with the anti-BCMA×CD3 bispecific antibody or the anti-CD20×CD3 bispecific antibody and in place of the FcRn blocker.

In some embodiments, a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle) described herein may be administered with an additional therapeutic agent comprising, e.g., a broad-spectrum immunosuppression methodology, or combination thereof, including broad spectrum immunosuppression (calcineurin inhibitors [tacrolimus, cyclosporine], rapamycin, MMF, corticosteroids, methotrexate, proteasome inhibitors, costimulation blockade [CTLA4-Ig/abatacept/belatacept], Src kinase inhibitors [dasatinib], Btk inhibitors [acalabrutinib]), B cell depleting agents (rituximab), IgG degrading enzymes (IdeS), IgG half-life reducers (FcRn blockers), or combinations thereof.

The additional therapeutically active component(s) may be administered just prior to, concurrent with, or shortly after the administration of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle), or the pharmaceutical composition(s) thereof, of the present disclosure. Such administration regimens can be considered, for example, the administration of a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle), or a pharmaceutical composition(s) thereof, “in combination with” an additional therapeutically active component.

The present disclosure includes pharmaceutical compositions in which a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle) of the present invention is co-formulated with one or more of the additional therapeutically active component(s) as described elsewhere herein.

Therapeutic or pharmaceutical compositions comprising the compositions or combinations disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

### XV. Nucleic Acid Constructs Encoding a Polypeptide of Interest

The compositions and methods described herein include the use of a nucleic acid construct that comprises a coding sequence for a polypeptide of interest (e.g., an exogenous polypeptide coding sequence). The compositions and methods described herein can also include the use of a nucleic acid construct that comprises a polypeptide of interest coding sequence or a reverse complement of the polypeptide of interest coding sequence (e.g., an exogenous polypeptide coding sequence or a reverse complement of the exogenous polypeptide coding sequence). Such nucleic acid constructs can be for insertion into a target genomic locus or into a cleavage site created by a nuclease agent or CRISPR/Cas system as disclosed elsewhere herein. The term cleavage site includes a DNA sequence at which a nick or double-strand break is created by a nuclease agent (e.g., a Cas9 protein complexed with a guide RNA). In some embodiments, a double-stranded break is created by a Cas9 protein complexed with a guide RNA, e.g., a Spy Cas9 protein complexed with a Spy Cas9 guide RNA. In some cases, the polypeptide of interest is an exogenous polypeptide as defined herein.

In a specific example, the compositions and methods described herein include the use of a nucleic acid construct encoding a Factor IX protein. Such nucleic acid constructs can be for insertion into a target genomic locus following cleavage at a cleavage site by a nuclease agent or CRISPR/Cas system as disclosed elsewhere herein or can be for expression of the Factor IX protein without insertion into a target genomic locus or a cleavage site (e.g., in an episome). The term cleavage site includes a DNA sequence at which a nick or double-strand break is created by a nuclease agent (e.g., a Cas9 protein complexed with a guide RNA). In certain embodiments, the cleavage site includes a DNA sequence at which a double-strand break is created by a Cas9 protein complexed with a guide RNA, e.g., a Spy Cas9 protein complexed with a Spy Cas9 guide RNA.

In another specific example, the compositions and methods described herein include the use of a nucleic acid construct that comprises a multidomain therapeutic protein (e.g., GAA fusion protein) coding sequence (a multidomain therapeutic protein nucleic acid). See, e.g., PCT/US2023/061858 and U.S. Ser. No. 18/163,698, each of which is herein incorporated by reference in its entirety for all purposes. Such nucleic acid constructs can be for insertion into a target genomic locus following cleavage at a cleavage site by a nuclease agent or CRISPR/Cas system as disclosed elsewhere herein or can be for expression of the multidomain therapeutic protein without insertion into a target genomic locus or a cleavage site (e.g., in an episome).

The length of the nucleic acid constructs disclosed herein can vary. The construct can be, for example, from about 1 kb to about 5 kb, such as from about 1 kb to about 4.5 kb or about 1 kb to about 4 kb. An exemplary nucleic acid construct is between about 1 kb to about 5 kb in length or between about 1 kb to about 4 kb in length. Alternatively, a nucleic acid construct can be between about 1 kb to about 1.5 kb, about 1.5 kb to about 2 kb, about 2 kb to about 2.5 kb, about 2.5 kb to about 3 kb, about 3 kb to about 3.5 kb, about 3.5 kb to about 4 kb, about 4 kb to about 4.5 kb, or about 4.5 kb to about 5 kb in length. Alternatively, a nucleic acid construct can be, for example, no more than 5 kb, no more than 4.5 kb, no more than 4 kb, no more than 3.5 kb, no more than 3 kb, or no more than 2.5 kb in length.

The constructs can comprise deoxyribonucleic acid (DNA) or ribonucleic acid (RNA), can be single-stranded, double-stranded, or partially single-stranded and partially double-stranded, and can be introduced into a host cell in linear or circular (e.g., minicircle) form. See, e.g., US 2010/0047805, US 2011/0281361, and US 2011/0207221, each of which is herein incorporated by reference in their entirety for all purposes. If introduced in linear form, the ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in their entirety for all purposes. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues. A construct can be introduced into a cell as part of a vector molecule having additional sequences such as, for example, replication origins, promoters, and genes encoding antibiotic resistance. A construct may omit viral elements. Moreover, constructs can be introduced as a naked nucleic acid, can be introduced as a nucleic acid complexed with an agent such as a liposome or poloxamer, or can be delivered by viruses (e.g., adenovirus, adeno-associated virus (AAV), herpesvirus, retrovirus, or lentivirus).

The constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Some constructs may be inserted so that their expression is driven by the endogenous promoter at the insertion site (e.g., the endogenous ALB promoter when the construct is integrated into the host cell's ALB locus). Such constructs may not comprise a promoter that drives the expression of the polypeptide of interest. For example, the expression of the polypeptide of interest can be driven by a promoter of the host cell (e.g., the endogenous ALB promoter when the transgene is integrated into a host cell's ALB locus). In such cases, the construct may lack control elements (e.g., promoter and/or enhancer) that drive its expression (e.g., a promoterless construct). Nonetheless, in other cases the construct may comprise a promoter and/or enhancer, for example, a constitutive promoter or an inducible or tissue-specific (e.g., liver- or platelet-specific) promoter that drives expression of the polypeptide of interest in an episome or upon integration. Non-limiting exemplary constitutive promoters include cytomegalovirus immediate early promoter (CMV), simian virus (SV40) promoter, adenovirus major late (MLP) promoter, Rous sarcoma virus (RSV) promoter, mouse mammary tumor virus (MMTV) promoter, phosphoglycerate kinase (PGK) promoter, elongation factor-alpha (EF1a) promoter, ubiquitin promoters, actin promoters, tubulin promoters, immunoglobulin promoters, a functional fragment thereof, or a combination of any of the foregoing. For example, the promoter may be a CMV promoter or a truncated CMV promoter. In another example, the promoter may be an EF1a promoter. Non-limiting exemplary inducible promoters include those inducible by heat shock, light, chemicals, peptides, metals, steroids, antibiotics, or alcohol. The inducible promoter may be one that has a low basal (non-induced) expression level, such as the Tet-On® promoter (Clontech). Although not required for expression, the constructs may comprise transcriptional or translational regulatory sequences such as promoters, enhancers, insulators, internal ribosome entry sites, additional sequences encoding peptides, and/or polyadenylation signals. The construct may comprise a sequence encoding a polypeptide of interest downstream of and operably linked to a signal sequence encoding a signal peptide. In some examples, the nucleic acid construct works in homology-independent insertion of a nucleic acid that encodes a polypeptide of interest. Such nucleic acid constructs can work, for example, in non-dividing cells (e.g., cells in which non-homologous end joining (NHEJ), not homologous recombination (HR), is the primary mechanism by which double-stranded DNA breaks are repaired) or dividing cells (e.g., actively dividing cells). Such constructs can be, for example, homology-independent donor constructs. In preferred embodiments, promoters and other regulatory sequences are appropriate for use in humans, e.g., recognized by regulatory factors in human cells, e.g., in human liver cells, and acceptable to regulatory authorities for use in humans.

The constructs disclosed herein can be modified to include or exclude any suitable structural feature as needed for any particular use and/or that confers one or more desired function. For example, some constructs disclosed herein do not comprise a homology arm. Some constructs disclosed herein are capable of insertion into a target genomic locus or a cut site in a target DNA sequence for a nuclease agent (e.g., capable of insertion into a safe harbor gene, such as an ALB locus) by non-homologous end joining. For example, such constructs can be inserted into a blunt end double-strand break following cleavage with a nuclease agent (e.g., CRISPR/Cas system, e.g., a SpyCas9 CRISPR/Cas system) as disclosed herein. In a specific example, the construct can be delivered via AAV and can be capable of insertion by non-homologous end joining (e.g., the construct does not comprise a homology arm).

In a particular example, the construct can be inserted via homology-independent targeted integration. For example, the polypeptide of interest coding sequence in the construct can be flanked on each side by a target site for a nuclease agent (e.g., the same target site as in the target DNA sequence for targeted insertion (e.g., in a safe harbor gene), and the same nuclease agent being used to cleave the target DNA sequence for targeted insertion). The nuclease agent can then cleave the target sites flanking the polypeptide of interest coding sequence. In a specific example, the construct is delivered AAV-mediated delivery, and cleavage of the target sites flanking the polypeptide of interest coding sequence can remove the inverted terminal repeats (ITRs) of the AAV. In some instances, the target DNA sequence for targeted insertion (e.g., target DNA sequence in a safe harbor locus such as a gRNA target sequence including the flanking protospacer adjacent motif) is no longer present if the polypeptide of interest coding sequence is inserted into the cut site or target DNA sequence in the correct orientation but it is reformed if the polypeptide of interest coding sequence is inserted into the cut site or target DNA sequence in the opposite orientation. This can help ensure that the polypeptide of interest coding sequence is inserted in the correct orientation for expression.

The constructs disclosed herein can comprise a polyadenylation sequence or polyadenylation tail sequence (e.g., downstream or 3′ of a polypeptide of interest coding sequence). Methods of designing a suitable polyadenylation tail sequence are well-known. The polyadenylation tail sequence can be encoded, for example, as a “poly-A” stretch downstream of the polypeptide of interest coding sequence. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. The term polyadenylation signal sequence refers to any sequence that directs termination of transcription and addition of a poly-A tail to the mRNA transcript. In eukaryotes, transcription terminators are recognized by protein factors, and termination is followed by polyadenylation, a process of adding a poly(A) tail to the mRNA transcripts in presence of the poly(A) polymerase. The mammalian poly(A) signal typically consists of a core sequence, about 45 nucleotides long, that may be flanked by diverse auxiliary sequences that serve to enhance cleavage and polyadenylation efficiency. The core sequence consists of a highly conserved upstream element (AATAAA or AAUAAA) in the mRNA, referred to as a poly A recognition motif or poly A recognition sequence), recognized by cleavage and polyadenylation-specificity factor (CPSF), and a poorly defined downstream region (rich in Us or Gs and Us), bound by cleavage stimulation factor (CstF). Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. In one example, the polyadenylation signal is a simian virus 40 (SV40) late polyadenylation signal. For example, the polyadenylation signal can comprise, consist essentially of, or consist of SEQ ID NO: 292 or 284. For example, the polyadenylation signal can comprise, consist essentially of, or consist of SEQ ID NO: 292. In another example, the polyadenylation signal is a bovine growth hormone (BGH) polyadenylation signal or a CpG depleted BGH polyadenylation signal. For example, the polyadenylation signal can comprise, consist essentially of, or consist of SEQ ID NO: 285.

In one example, the polyadenylation signal can comprise a BGH polyadenylation signal. For example, the BGH polyadenylation signal can comprise, consist essentially of, or consist of SEQ ID NO: 858. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 859. In another example, a synthetic polyadenylation signal can be used. For example, the synthetic polyadenylation signal can comprise, consist essentially of, or consist of SEQ ID NO: 860. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 858, and the unidirectional SV40 late polyadenylation signal can comprise, consist essentially of, or consist of SEQ ID NO: 859. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 902. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 858, and the synthetic polyadenylation signal can comprise, consist essentially of, or consist of SEQ ID NO: 860. In some embodiments, the nucleic acid construct is a unidirectional construct.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 859. In some embodiments, the unidirectional SV40 late polyadenylation signal comprises, consists essentially of, or consists of the sequence set forth in SEQ ID NO: 859.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 858. In some embodiments, the BGH polyadenylation signal comprises, consists essentially of, or consists of the sequence set forth in SEQ ID NO: 858. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 902. In some embodiments, the combination of the BGH polyadenylation signal and the unidirectional SV40 late polyadenylation signal comprises, consists essentially of, or consists of the sequence set forth in SEQ ID NO: 902.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

The constructs disclosed herein may also comprise splice acceptor sites (e.g., operably linked to the polypeptide of interest coding sequence, such as upstream or 5′ of the polypeptide of interest coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest (e.g., a GAA splice acceptor). For example, such a splice acceptor can be derived from the human GAA gene. Alternatively, such a splice acceptor can be derived from the mouse GAA gene. Additional suitable splice acceptor sites useful in eukaryotes, including artificial splice acceptors, are well-known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. In a specific example, the splice acceptor is a mouse Alb exon 2 splice acceptor. In a specific example, the splice acceptor can comprise, consist essentially of, or consist of SEQ ID NO: 286.

In some examples, the nucleic acid constructs disclosed herein can be bidirectional constructs, which are described in more detail below. In some examples, the nucleic acid constructs disclosed herein can be unidirectional constructs, which are described in more detail below. Likewise, in some examples, the nucleic acid constructs disclosed herein can be in a vector (e.g., viral vector, such as AAV, or rAAV8) and/or a lipid nanoparticle as described in more detail elsewhere herein.

### A. Polypeptides of Interest

Any polypeptide of interest may be encoded by the nucleic acid constructs disclosed herein. In one example, the polypeptide of interest is a therapeutic polypeptide (e.g., a polypeptide that is lacking or deficient in a subject). In one example, the polypeptide of interest is an enzyme.

The polypeptide of interest can be a secreted polypeptide (e.g., a protein that is secreted by the cell and/or is functionally active as a soluble extracellular protein). Alternatively, the polypeptide of interest can be an intracellular polypeptide (e.g., a protein that is not secreted by the cell and is functionally active within the cell, including soluble cytosolic polypeptides).

The polypeptide of interest can be a wild type polypeptide. Alternatively, the polypeptide of interest can be a variant or mutant polypeptide.

In one example, the polypeptide of interest is a liver protein (e.g., a protein that is, endogenously produced in the liver and/or functionally active in the liver). In another example, the polypeptide of interest can be a circulating protein that is produced by the liver. In another example, the polypeptide of interest can be a non-liver protein.

The polypeptide of interest can be an exogenous polypeptide. An “exogenous” polypeptide coding sequence can refer to a coding sequence that has been introduced from an exogenous source to a site within a host cell genome (e.g., at a genomic locus such as a safe harbor locus, including ALB intron 1). That is, the exogenous polypeptide coding sequence is exogenous with respect to its insertion site, and the polypeptide of interest expressed from such an exogenous coding sequence is referred to as an exogenous polypeptide. The exogenous coding sequence can be naturally-occurring or engineered, and can be wild type or a variant. The exogenous coding sequence may include nucleotide sequences other than the sequence that encodes the exogenous polypeptide (e.g., an internal ribosomal entry site). The exogenous coding sequence can be a coding sequence that occurs naturally in the host genome, as a wild type or a variant (e.g., mutant). For example, although the host cell contains the coding sequence of interest (as a wild type or as a variant), the same coding sequence or variant thereof can be introduced as an exogenous source (e.g., for expression at a locus that is highly expressed). The exogenous coding sequence can also be a coding sequence that is not naturally occurring in the host genome, or that expresses an exogenous polypeptide that does not naturally occur in the host genome. An exogenous coding sequence can include an exogenous nucleic acid sequence (e.g., a nucleic acid sequence is not endogenous to the recipient cell), or may be exogenous with respect to its insertion site and/or with respect to its recipient cell.

In one example, the polypeptide of interest is a factor IX protein. See, e.g., WO 2023/077012 and US 2023-0149563, each of which is herein incorporated by reference in its entirety for all purposes.

In one example, the polypeptide of interest is a multidomain therapeutic protein comprising a CD63-binding delivery domain linked to or fused to a lysosomal alpha-glucosidase (GAA). See, e.g., PCT/US2023/061858 and U.S. Ser. No. 18/163,698, each of which is herein incorporated by reference in its entirety for all purposes. In one example, the polypeptide of interest is a multidomain therapeutic protein comprising a CD63-binding delivery domain fused to a lysosomal alpha-glucosidase (GAA). In one example, the polypeptide of interest is a multidomain therapeutic protein comprising a TfR-binding delivery domain linked to or fused to a GAA. See, e.g., PCT/US2023/061858 and U.S. Ser. No. 18/163,698, each of which is herein incorporated by reference in its entirety for all purposes. In one example, the polypeptide of interest is a multidomain therapeutic protein comprising a TfR-binding delivery domain fused to a GAA.

In one example, the polypeptide of interest is a polypeptide associated with a genetic enzyme deficiency. In certain embodiments, the genetic enzyme deficiency results in infantile onset of disease. In certain embodiments, the genetic enzyme deficiency can be, or routinely is, diagnosed with newborn screening. In certain embodiments, the enzyme deficiency may manifest in various severity of disease such that the age of onset may include an infantile onset form of the disease and a later onset form of the disease (e.g., childhood, adolescent, or adult form of onset).

In one example, the polypeptide of interest is a polypeptide associated with a bleeding disorder, e.g., hemophilia, e.g., hemophilia A or hemophilia B. In certain embodiments, the polypeptide of interest is Factor VIII or Factor IX. In one example, the polypeptide of interest is an enzyme related to inborn errors of metabolism. In one example, the polypeptide of interest is an enzyme related to a lysosomal storage disease.

In another example, the polypeptide of interest is a multidomain therapeutic protein. A multidomain therapeutic protein as described herein includes a lysosomal alpha-glucosidase (GAA; e.g., to provide GAA enzyme replacement activity) linked to or fused to a delivery domain that provides binding to an internalization effector (a protein that is capable of being internalized into a cell or that otherwise participates in or contributes to retrograde membrane trafficking). Examples of multidomain therapeutic proteins can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes. For example, the multidomain therapeutic proteins described herein can comprise a CD63-binding delivery domain linked to or fused to a lysosomal alpha-glucosidase (GAA). CD63-binding domains and GAA are described in more detail below. The CD63-binding domain provides binding to the internalization factor CD63. The multidomain therapeutic protein is targeted to the muscle by targeting CD63, which is a rapidly internalizing protein highly expressed in the muscle. In some multidomain therapeutic proteins, the CD63-binding delivery domain is covalently linked to the GAA. The covalent linkage may be any type of covalent bond (i.e., any bond that involved sharing of electrons). In some cases, the covalent bond is a peptide bond between two amino acids, such that the GAA and the CD63-binding delivery domain in whole or in part form a continuous polypeptide chain, as in a fusion protein. In some cases, the GAA portion and the CD63-binding delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the CD63-binding delivery domain and the GAA to facilitate removal of the CD63-binding delivery domain in the lysosome. In another example, the linker can comprise an amino acid sequence, e.g., about 10 amino acids in length, for example, 1, 2, 3, 4, 5, 6, 7, 8, 8, or 10 repeats of Gly₄Ser (SEQ ID NO: 718). In one example, the linker comprises, consists essentially of, or consists of three such repeats (SEQ ID NO: 828). For example, the coding sequence for the linker can comprise, consist essentially of, or consist of any one of SEQ ID NOS: 830-834 and 854. In another example, the linker comprises, consists essentially of, or consists of two such repeats (SEQ ID NO: 829). For example, the coding sequence for the linker can comprise, consist essentially of, or consist of any one of SEQ ID NOS: 835-841. In another example, the linker comprises, consists essentially of, or consists of one such repeat (SEQ ID NO: 718). For example, the coding sequence for the linker can comprise, consist essentially of, or consist of SEQ ID NO: 842 or 855.

In a particular multidomain therapeutic protein, the GAA is covalently linked to the C-terminus of the heavy chain of an anti-CD63 antibody or to the C-terminus of the light chain. In another particular multidomain therapeutic protein, the GAA is covalently linked to the N-terminus of the heavy chain of an anti-CD63 antibody or to the N-terminus of the light chain. In another particular embodiment, the GAA is linked to the C-terminus of an anti-CD63 scFv domain.

As another example, the multidomain therapeutic proteins described herein can comprise a TfR-binding delivery domain linked to or fused to a lysosomal alpha-glucosidase (GAA). TfR-binding domains and GAA are described in more detail below. The TfR-binding domain provides binding to the internalization factor TfR. The multidomain therapeutic protein produced by the liver is targeted the muscle and CNS by targeting TfR, which is expressed in muscle and on brain endothelial cells. Transcytosis of TfR in these cells enables blood-brain-barrier crossing. In some multidomain therapeutic proteins, the TfR-binding delivery domain is covalently linked to the GAA. The covalent linkage may be any type of covalent bond (i.e., any bond that involved sharing of electrons). In some cases, the covalent bond is a peptide bond between two amino acids, such that the GAA and the TfR-binding delivery domain in whole or in part form a continuous polypeptide chain, as in a fusion protein. In some cases, the GAA portion and the TfR-binding delivery domain portion are directly linked. In other cases, a linker, such as a peptide linker, is used to tether the two portions. Any suitable linker can be used. See Chen et al., “Fusion protein linkers: property, design and functionality,” 65(10) Adv Drug Deliv Rev. 1357-69 (2013). In some cases, a cleavable linker is used. For example, a cathepsin cleavable linker can be inserted between the TfR-binding delivery domain and the GAA to facilitate removal of the TfR-binding delivery domain in the lysosome. In another example, the linker can comprise an amino acid sequence, e.g., about 10 amino acids in length, for example, 1, 2, 3, 4, 5, 6, 7, 8, 8, or 10 repeats of Gly₄Ser (SEQ ID NO: 718). In one example, the linker comprises, consists essentially of, or consists of three such repeats (SEQ ID NO: 828). For example, the coding sequence for the linker can comprise, consist essentially of, or consist of any one of SEQ ID NOS: 830-834. In another example, the linker comprises, consists essentially of, or consists of two such repeats (SEQ ID NO: 829). For example, the coding sequence for the linker can comprise, consist essentially of, or consist of any one of SEQ ID NOS: 835-841. In another example, the linker comprises, consists essentially of, or consists of one such repeat (SEQ ID NO: 718). For example, the coding sequence for the linker can comprise, consist essentially of, or consist of SEQ ID NO: 842.

In a particular multidomain therapeutic protein, the GAA is covalently linked to the C-terminus of the heavy chain of an anti-TfR antibody or to the C-terminus of the light chain. In another particular multidomain therapeutic protein, the GAA is covalently linked to the N-terminus of the heavy chain of an anti-TfR antibody or to the N-terminus of the light chain. In another particular embodiment, the GAA is linked to the C-terminus of an anti-TfR scFv domain.

In another example, the polypeptide of interest is an antigen-binding protein. See, e.g., WO 2020/206162 and US 2020-0318136, each of which is herein incorporated by reference in its entirety for all purposes. An “antigen-binding protein” as disclosed herein includes any protein that binds to an antigen. Examples of antigen-binding proteins include an antibody, an antigen-binding fragment of an antibody, a multi-specific antibody (e.g., a bi-specific antibody), an scFv, a bis-scFv, a diabody, a triabody, a tetrabody, a V-NAR, a VHH, a VL, a F(ab), a F(ab)₂, a DVD (dual variable domain antigen-binding protein), an SVD (single variable domain antigen-binding protein), a bispecific T-cell engager (BiTE), or a Davisbody (U.S. Pat. No. 8,586,713, herein incorporated by reference herein in its entirety for all purposes).

An antigen-binding protein or antibody can be, for example, a neutralizing antigen-binding protein or antibody or a broadly neutralizing antigen-binding protein or antibody. A neutralizing antibody is an antibody that defends a cell from an antigen or infectious body by neutralizing any effect it has biologically. Broadly-neutralizing antibodies (bNAbs) affect multiple strains of a particular bacteria or virus. For example, broadly neutralizing antibodies can focus on conserved functional targets, attacking a vulnerable site on conserved bacterial or viral proteins (e.g., a vulnerable site on the influenza viral protein hemagglutinin). Antibodies developed by the immune system upon infection or vaccination tend to focus on easily accessible loops on the bacterial or viral surface, which often have great sequence and conformational variability. This is a problem for two reasons: the bacteria or virus population can quickly evade these antibodies, and the antibodies are attacking portions of the protein that are not essential for function. Broadly neutralizing antibodies—termed “broadly” because they attack many strains of the bacteria or virus, and “neutralizing” because they attack key functional sites in the bacteria or virus and block infection—can overcome these problems. Unfortunately, however, these antibodies usually come too late and do not provide effective protection from the disease.

The antigen-binding proteins disclosed herein can target any antigen. The term “antigen” refers to a substance, whether an entire molecule or a domain within a molecule, which is capable of eliciting production of antibodies with binding specificity to that substance. The term antigen also includes substances, which in wild type host organisms would not elicit antibody production by virtue of self-recognition, but can elicit such a response in a host animal with appropriate genetic engineering to break immunological tolerance.

As one example, the targeted antigen can be a disease-associated antigen. The term “disease-associated antigen” refers to an antigen whose presence is correlated with the occurrence or progression of a particular disease. For example, the antigen can be in a disease-associated protein (i.e., a protein whose expression is correlated with the occurrence or progression of the disease). Optionally, a disease-associated protein can be a protein that is expressed in a particular type of disease but is not normally expressed in healthy adult tissue (i.e., a protein with disease-specific expression or disease-restricted expression). However, a disease-associated protein does not have to have disease-specific or disease-restricted expression.

As one example, a disease-associated antigen can be a cancer-associated antigen. The term “cancer-associated antigen” refers to an antigen whose presence is correlated with the occurrence or progression of one or more types of cancer. For example, the antigen can be in a cancer-associated protein (i.e., a protein whose expression is correlated with the occurrence or progression of one or more types of cancer). For example, a cancer-associated protein can be an oncogenic protein (i.e., a protein with activity that can contribute to cancer progression, such as proteins that regulate cell growth), or it can be a tumor-suppressor protein (i.e., a protein that typically acts to alleviate the potential for cancer formation, such as through negative regulation of the cell cycle or by promoting apoptosis). Optionally, a cancer-associated protein can be a protein that is expressed in a particular type of cancer but is not normally expressed in healthy adult tissue (i.e., a protein with cancer-specific expression, cancer-restricted expression, tumor-specific expression, or tumor-restricted expression). However, a cancer-associated protein does not have to have cancer-specific, cancer-restricted, tumor-specific, or tumor-restricted expression. Examples of proteins that are considered cancer-specific or cancer-restricted are cancer testis antigens or oncofetal antigens. Cancer testis antigens (CTAs) are a large family of tumor-associated antigens expressed in human tumors of different histological origin but not in normal tissue, except for male germ cells. In cancer, these developmental antigens can be re-expressed and can serve as a locus of immune activation. Oncofetal antigens (OFAs) are proteins that are typically present only during fetal development but are found in adults with certain kinds of cancer.

As another example, a disease-associated antigen can be an infectious-disease-associated antigen. The term “infectious-disease-associated antigen” refers to an antigen whose presence is correlated with the occurrence or progression of a particular infectious disease. For example, the antigen can be in an infectious-disease-associated protein (i.e., a protein whose expression is correlated with the occurrence or progression of the infectious disease). Optionally, an infectious-disease-associated protein can be a protein that is expressed in a particular type of infectious disease but is not normally expressed in healthy adult tissue (i.e., a protein with infectious-disease-specific expression or infectious-disease-restricted expression). However, an infectious-disease-associated protein does not have to have infectious-disease-specific or infectious-disease-restricted expression. For example, the antigen can be a viral antigen or a bacterial antigen. Such antigens include, for example, molecular structures on the surface of viruses or bacteria (e.g., viral proteins or bacterial proteins) that are recognized by the immune system and are capable of triggering an immune response.

Examples of viral antigens include antigens within proteins expressed by the Zika virus or influenza (flu) viruses. Zika is a virus spread to people primarily through the bite of an infected Aedes species mosquito (Ae. aegypti and Ae. Albopictus). Zika virus infection during pregnancy can cause microcephaly and other severe brain defects. For example, a Zika antigen can be, but is not limited to, an antigen within a Zika virus envelope (Env) protein. Influenza virus is a virus that causes an infectious disease called influenza (commonly known as “the flu”). Three types of influenza viruses affect people, called Type A, Type B, and Type C. An influenza antigen can be, but is not limited to, an antigen within the hemagglutinin protein. Viral antigens and bacterial antigens also include antigens on other viruses and other bacteria. Examples of antibodies targeting influenza hemagglutinin are provided, e.g., in WO 2016/100807, herein incorporated by reference in its entirety for all purposes.

Examples of bacterial antigens include antigens within proteins expressed by Pseudomonas aeruginosa (e.g., an antigen within PcrV, which is a type III virulence system translocating protein). Pseudomonas aeruginosa is an opportunistic bacterial pathogen that causes fatal acute lung infections in critically ill individuals. Its pathogenesis is associated with bacterial virulence conferred by the type III secretion system (TTSS), through which P. aeruginosa causes necrosis of the lung epithelium and disseminates into the circulation, resulting in bacteremia, sepsis, and mortality. TTSS allows P. aeruginosa to directly translocate cytotoxins into eukaryotic cells, inducing cell death. The P. aeruginosa V-antigen PcrV, a homolog of the Yersinia V-antigen LcrV, is an indispensable contributor to TTS toxin translocation.

The antigen-binding protein can be a single-chain antigen-binding protein such as an scFv. Alternatively, the antigen-binding protein is not a single-chain antigen-binding protein. For example, the antigen-binding protein can include separate light and heavy chains. The heavy chain coding sequence can be upstream of the light chain coding sequence, or the light chain coding sequence can be upstream of the heavy chain coding sequence. In one specific example, the heavy chain coding sequence is upstream of the light chain coding sequence. For example, the heavy chain coding sequence can comprise V_{H}, D_{H}, and J_{H} segments, and the light chain coding sequence can comprise light chain V_{L} and light chain J_{L} gene segments. The antigen-binding protein coding sequence can be operably linked to an exogenous promoter in the nucleic acid construct, or the nucleic acid construct can be designed such that the antigen-binding protein coding sequence will be operably linked to an endogenous promoter at the genomic locus or safe harbor locus once it is genomically integrated. In one specific example, the nucleic acid construct can be designed such that the antigen-binding protein coding sequence will be operably linked to an endogenous promoter at the genomic locus or safe harbor locus once it is genomically integrated. Likewise, the antigen-binding protein coding sequence in the nucleic acid construct can include an exogenous signal sequence for secretion and/or the nucleic acid construct can be designed so that the antigen-binding protein coding sequence will be operably linked to an endogenous signal sequence at the genomic locus or safe harbor locus once it is genomically integrated. In one example, the nucleic acid construct can be designed so that the antigen-binding protein coding sequence will be operably linked to an endogenous signal sequence at the genomic locus or safe harbor locus once it is genomically integrated. In a specific example, the antigen-binding protein comprises separate light and heavy chains, and the nucleic acid construct is designed such that the coding sequence for one chain will be operably linked to an endogenous signal sequence at the genomic locus or safe harbor locus once it is genomically integrated and the coding sequence for the other chain is operably linked to a separate exogenous signal sequence. In a specific example, the antigen-binding protein comprises separate light and heavy chains, and the nucleic acid construct is designed such that the whichever chain coding sequence is upstream in the nucleic acid construct will be operably linked to an endogenous signal sequence at the genomic locus or safe harbor locus once it is genomically integrated, and an exogenous signal sequence is operably linked to the whichever chain coding sequence is downstream in the exogenous donor nucleic acid. Alternatively, the nucleic acid construct can be designed such that the coding sequences for both chains will be operably linked to an endogenous signal sequence at the genomic locus or safe harbor locus once it is genomically integrated, or the coding sequence for both chains can be operably linked to the same exogenous signal sequence or the coding sequence for each chain can be operably linked to separate exogenous signal sequences.

Signal sequences (i.e., N-terminal signal sequences) mediate targeting of nascent secretory and membrane proteins to the endoplasmic reticulum (ER) in a signal recognition particle (SRP)-dependent manner. Usually, signal sequences are cleaved off co-translationally so that signal peptides and mature proteins are generated. Examples of exogenous signal sequences or signal peptides that can be used include, for example, the signal sequence/peptide from mouse albumin, human albumin, mouse ROR1, human ROR1, human azurocidin, Cricetulus griseus Ig kappa chain V III region MOPC 63 like, and human Ig kappa chain V III region VG. Any other known signal sequence/peptide can also be used.

One or more of the nucleic acids in the antigen-binding-protein coding sequence (e.g., a heavy chain coding sequence and a light chain coding sequence) can be together in a multicistronic expression construct. For example, a nucleic acid encoding a heavy chain and a light chain can be together in a bicistronic expression construct. Multicistronic expression vectors simultaneously express two or more separate proteins from the same mRNA (i.e., a transcript produced from the same promoter). Suitable strategies for multicistronic expression of proteins include, for example, the use of a 2A peptide and the use of an internal ribosome entry site (IRES). As one example, such multicistronic vectors can use one or more internal ribosome entry sites (IRES) to allow for initiation of translation from an internal region of an mRNA. As another example, such multicistronic vectors can use one or more 2A peptides. These peptides are small “self-cleaving” peptides, generally having a length of 18-22 amino acids and produce equimolar levels of multiple genes from the same mRNA. Ribosomes skip the synthesis of a glycyl-prolyl peptide bond at the C-terminus of a 2A peptide, leading to the “cleavage” between a 2A peptide and its immediate downstream peptide. See, e.g., Kim et al. (2011) PLoS One 6(4): e18556, herein incorporated by reference in its entirety for all purposes. The “cleavage” occurs between the glycine and proline residues found on the C-terminus, meaning the upstream cistron will have a few additional residues added to the end, while the downstream cistron will start with the proline. As a result, the “cleaved-off” downstream peptide has proline at its N-terminus. 2A-mediated cleavage is a universal phenomenon in all eukaryotic cells. 2A peptides have been identified from picornaviruses, insect viruses and type C rotaviruses. See, e.g., Szymczak et al. (2005) Expert Opin Biol Ther 5:627-638, herein incorporated by reference in its entirety for all purposes. Examples of 2A peptides that can be used include Thosea asigna virus 2A (T2A); porcine teschovirus-1 2A (P2A); equine rhinitis A virus (ERAV) 2A (E2A); and FMDV 2A (F2A). GSG residues can be added to the 5′ end of any of these peptides to improve cleavage efficiency.

In some nucleic acid constructs, a nucleic acid encoding a furin cleavage site is included between the light chain coding sequence and the heavy chain coding sequence. In some nucleic acid construct, a nucleic acid encoding a linker (e.g., GSG) is included between the light chain coding sequence and the heavy chain coding sequence (e.g., directly upstream of the 2A peptide coding sequence). For example, a furin cleavage site can be included upstream of a 2A peptide, with both the furin cleavage site and the 2A peptide being located between the light chain and the heavy chain (i.e., upstream chain-furin cleavage site-2A peptide-downstream chain). During translation, a first cleavage event will occur at the 2A peptide sequence. However, most of the 2A peptide will remain attached as a remnant to the C-terminus of the upstream chain (e.g., light chain if the light chain is upstream of the heavy chain, or heavy chain if the heavy chain is upstream of the light chain), with one amino acid added to the N-terminus of the downstream chain (or the N-terminus of a signal sequence, if a signal sequence is included upstream of the downstream chain). A second cleavage event, initiated at the furin cleavage site, yields the upstream chain without the 2A remnants in order to obtain a more native heavy chain or light chain by post-translational processing.

### (1) Factor IX

Coagulation factor IX (FIX; also known as Christmas factor or plasma thromboplastin component or PTC) is encoded by factor 9 (F9) and is a 415-amino acid serine protease synthesized in the liver. It is a vitamin K-dependent plasma protein that participates in the intrinsic pathway of blood coagulation by converting factor X to its active form in the presence of Ca²⁺ ions, phospholipids, and factor VIIIa. The plasma concentration of FIX is about 50 times that of factor VIII, and FIX has a half-life of about 24 hours.

The FIX expressed from the compositions and methods disclosed herein can be any wild type or variant FIX. In one example, the FIX is a human FIX protein. Human FIX is assigned UniProt reference number P00740. An exemplary amino acid sequence for human Factor IX is assigned NCBI Accession No. NP_000124.1 and is set forth in SEQ ID NO: 57. An exemplary human F9 mRNA (cDNA) sequence is assigned NCBI Accession No. NM_000133.4 and is set forth in SEQ ID NO: 58. An exemplary human F9 coding sequence is assigned CCDS ID CCDS14666.1 and is set forth in SEQ ID NO: 59.

In some examples, the FIX (e.g., human FIX) is a wild type FIX (e.g., wild type human FIX) sequence or a fragment thereof. For example, the FIX can be a fragment comprising the mature FIX amino acid sequence (i.e., the FIX sequence after removal of the signal peptide and propeptide), or a fragment comprising the mature FIX amino acid sequence and a portion of the propeptide. In a specific example, the FIX can comprise SEQ ID NO: 97 or can be at least 90%, at least 91%, at least 92%, at least 93%, at least 94%, at least 95%, at least 96%, at least 97%, at least 98%, at least 99%, or at least 99.5% identical to SEQ ID NO: 97.

In some examples, the FIX (e.g., human FIX) is not a hyperactive or hyperfunctional variant of FIX (i.e., the FIX does not have one or more mutations that increase the activity of the variant FIX relative to wild type). In other examples, the FIX (e.g., human FIX) is not a FVIII-independent variant of FIX (i.e., the FIX does not have one or more mutations that allow the variant FIX to activate coagulation in the absence of its cofactor, factor VIII). In other examples, the FIX (e.g., human FIX) is not a hyperactive or hyperfunctional variant of FIX and is not a FVIII-independent variant of FIX.

In other examples, the FIX (e.g., human FIX) is a variant FIX (e.g., a variant human FIX) or a fragment thereof. For example, the variant FIX or fragment thereof can comprise one or more mutations. In one example, the variant FIX or fragment thereof can have one or more mutations that increase the activity of the variant FIX (hyperactive or hyperfunctional) relative to wild type, such as an amino acid substitution in position R338 (e.g., R338A or R338L) and/or an amino acid substitution at position S377 (e.g., S377W). See, e.g., US 2019/0017039 and US 2020/0172892, each of which is herein incorporated by reference in its entirety for all purposes. The numbering referred to herein is the standard FIX numbering, with position 1 being the tyrosine at amino acid 47 in SEQ ID NO: 57 (i.e., the first amino acid of the mature FIX protein following the signal peptide and propeptide in SEQ ID NO: 57). Further examples of variant FIX comprise an amino acid at residue 338 chosen from alanine, leucine, valine, isoleucine, phenylalanine, tryptophan, methionine, serine, and threonine. Further FIX variants comprise an amino acid at residue 338 chosen from leucine, cysteine, aspartic acid, glutamic acid, histidine, lysine, asparagine, glutamine, or tyrosine. In another example, the variant FIX or fragment thereof can have one or more mutations that allow the variant FIX to activate coagulation in the absence of its cofactor, factor VIII, such as an amino acid substitution at position L6, V181, E185, Y259, A261, K265, Y345, 1383, E388, or a combination thereof (e.g., L6F, V181I, E185D, E185S, Y259F, A261K, K265A, K265T, Y345F, I383V, E188G, or a combination thereof). See, e.g., U.S. Pat. Nos. 10,125,357, 10,000,748, 10,604,749, US 2008/0214462, U.S. Pat. Nos. 8,022,187, and 8,513,386, each of which is herein incorporated by reference in its entirety for all purposes. In another example, the variant FIX or fragment thereof can have one or more mutations that allow the variant FIX to activate coagulation in the absence of its cofactor, factor VIII, such as an amino acid substitution at position V181, K265, 1383, or a combination thereof or at position L6, V181, K265, 1383, E185, or a combination thereof (e.g., an L6F mutation, a V181I mutation, a K265A or K265T mutation, an I383V mutation, an E185D mutation, or a combination thereof such as L6F/V181I/K265A/I383V, L6F/V181I/K265T/I383V, V181I/K265A/I383V/E185D, V181I/K265T/1383V/E185D, V181I/K265A/I383V/E185S, or V181I/K265T/I383V/E185S, or a V181I mutation, a K265A or K265T mutation, an I383V mutation, or a combination thereof such as V181I/K265A/I383V or V181I/K265T/I383V). In another example, the variant FIX or fragment thereof can have one or more mutations that increase the activity of the variant FIX relative to wild type and one or more mutations that allow the variant FIX to activate coagulation in the absence of its cofactor, factor VIII.

The FIX coding sequences in the constructs disclosed herein may include wild type FIX coding sequences without any modifications. The FIX coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a construct disclosed herein has one or more cryptic splice sites mutated or removed. In another example, a FIX coding sequence in a construct disclosed herein has all identified cryptic splice sites mutated or removed. In another example, a FIX coding sequence in a construct disclosed herein has one or more CpG dinucleotides removed (i.e., is CpG depleted). In another example, a FIX coding sequence in a construct disclosed herein has all but one CpG dinucleotides removed. In another example, a FIX coding sequence in a construct disclosed herein has all CpG dinucleotides removed (i.e., is fully CpG depleted). In another example, a FIX coding sequence in a construct disclosed herein is codon optimized (e.g., codon optimized for expression in a human or mammal). In a specific example, a FIX coding sequence in a construct disclosed herein has one or more CpG dinucleotides removed (i.e., is CpG depleted) and has one or more cryptic splice sites mutated or removed. In another specific example, a FIX coding sequence in a construct disclosed herein has all but one CpG dinucleotides removed (e.g., introducing one CpG to mutate a cryptic splice site) and has one or more or all identified cryptic splice sites mutated or removed. In another specific example, a FIX coding sequence in a construct disclosed herein has one or more CpG dinucleotides removed (i.e., is CpG depleted) and is codon optimized (e.g., codon optimized for expression in a human or mammal). In another specific example, a FIX coding sequence in a construct disclosed herein has all CpG dinucleotides removed (i.e., is fully CpG depleted) and is codon optimized (e.g., codon optimized for expression in a human or mammal).

Various codon optimized FIX coding sequences are provided. See, e.g., WO 2023/077012 and US 2023-0149563, each of which is herein incorporated by reference in its entirety for all purposes. The FIX coding sequence can be, for example, CpG-depleted (e.g., fully CpG depleted) and/or codon optimized (e.g., CpG depleted (e.g., fully CpG-depleted) and codon optimized). In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 64-73. In another example, the FIX coding sequence is (or comprises a sequence) at least 95%, at least 96%, at least 97%, at least 98%, at least 99%, at least 99.5%, or 100% identical to any one of SEQ ID NOS: 64-73. In another example, the FIX coding sequence is (or comprises a sequence) at least 99%, at least 99.5%, or 100% identical to any one of SEQ ID NOS: 64-73. In another example, the FIX coding sequence comprises the sequence set forth in any one of SEQ ID NOS: 64-73. In another example, the FIX coding sequence consists essentially of the sequence set forth in any one of SEQ ID NOS: 64-73. In another example, the FIX coding sequence consists of the sequence set forth in any one of SEQ ID NOS: 64-73. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 66-73. In another example, the FIX coding sequence is (or comprises a sequence) at least 95%, at least 96%, at least 97%, at least 98%, at least 99%, at least 99.5%, or 100% identical to any one of SEQ ID NOS: 66-73. In another example, the FIX coding sequence is (or comprises a sequence) at least 99%, at least 99.5%, or 100% identical to any one of SEQ ID NOS: 66-73. In another example, the FIX coding sequence comprises the sequence set forth in any one of SEQ ID NOS: 66-73. In another example, the FIX coding sequence consists essentially of the sequence set forth in any one of SEQ ID NOS: 66-73. In another example, the FIX coding sequence consists of the sequence set forth in any one of SEQ ID NOS: 66-73. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 or 67. In another example, the FIX coding sequence is (or comprises a sequence) at least 95%, at least 96%, at least 97%, at least 98%, at least 99%, at least 99.5%, or 100% identical to SEQ ID NO: 68 or 67. In another example, the FIX coding sequence is (or comprises a sequence) at least 99%, at least 99.5%, or 100% identical to SEQ ID NO: 68 or 67. In another example, the FIX coding sequence comprises the sequence set forth in SEQ ID NO: 68 or 67. In another example, the FIX coding sequence consists essentially of the sequence set forth in SEQ ID NO: 68 or 67. In another example, the FIX coding sequence consists of the sequence set forth in SEQ ID NO: 68 or 67. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 68. In another example, the FIX coding sequence is (or comprises a sequence) at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. In another example, the FIX coding sequence is (or comprises a sequence) at least 99%, at least 99.5%, or 100% identical to SEQ ID NO: 68. In another example, the FIX coding sequence is (or comprises a sequence) at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the FIX coding sequence is (or comprises a sequence) at least 99%, at least 99.5%, or 100% identical to SEQ ID NO: 68 and encodes a FIX protein comprising the sequence set forth in SEQ ID NO: 97. In another example, the FIX coding sequence comprises the sequence set forth in SEQ ID NO: 68. In another example, the FIX coding sequence consists essentially of the sequence set forth in SEQ ID NO: 68. In another example, the FIX coding sequence consists of the sequence set forth in SEQ ID NO: 68. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 67. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 67 and encodes a FIX protein (or a FIX protein comprising a sequence) at least 99%, at least 99.5%, or 100% identical to SEQ ID NO: 97. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 67 and encodes a FIX protein comprising the sequence set forth in SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 67. In another example, the FIX coding sequence is (or comprises a sequence) at least 95%, at least 96%, at least 97%, at least 98%, at least 99%, at least 99.5%, or 100% identical to SEQ ID NO: 67 and encodes a FIX protein (or a FIX protein comprising a sequence) at least 99%, at least 99.5%, or 100% identical to SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 67 and encodes a FIX protein comprising the sequence set forth in SEQ ID NO: 97. In another example, the FIX coding sequence is (or comprises a sequence) at least 99%, at least 99.5%, or 100% identical to SEQ ID NO: 67. In another example, the FIX coding sequence is (or comprises a sequence) at least 99%, at least 99.5%, or 100% identical to SEQ ID NO: 67 and encodes a FIX protein (or a FIX protein comprising a sequence) at least 99%, at least 99.5%, or 100% identical to SEQ ID NO: 97. In another example, the FIX coding sequence is (or comprises a sequence) at least 99%, at least 99.5%, or 100% identical to SEQ ID NO: 67 and encodes a FIX protein comprising the sequence set forth in SEQ ID NO: 97. In another example, the FIX coding sequence comprises the sequence set forth in SEQ ID NO: 67. In another example, the FIX coding sequence consists essentially of the sequence set forth in SEQ ID NO: 67. In another example, the FIX coding sequence consists of the sequence set forth in SEQ ID NO: 67. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

Various native and optimized native FIX coding sequences are also provided. In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 60-63. In another example, the FIX coding sequence is (or comprises a sequence) at least 95%, at least 96%, at least 97%, at least 98%, at least 99%, at least 99.5%, or 100% identical to any one of SEQ ID NOS: 60-63. In another example, the FIX coding sequence is (or comprises a sequence) at least 99%, at least 99.5%, or 100% identical to any one of SEQ ID NOS: 60-63. In another example, the FIX coding sequence comprises the sequence set forth in any one of SEQ ID NOS: 60-63. In another example, the FIX coding sequence consists essentially of the sequence set forth in any one of SEQ ID NOS: 60-63. In another example, the FIX coding sequence consists of the sequence set forth in any one of SEQ ID NOS: 60-63. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

Various optimized native FIX coding sequences are also provided. In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 61-63. In another example, the FIX coding sequence is (or comprises a sequence) at least 95%, at least 96%, at least 97%, at least 98%, at least 99%, at least 99.5%, or 100% identical to any one of SEQ ID NOS: 61-63. In another example, the FIX coding sequence is (or comprises a sequence) at least 99%, at least 99.5%, or 100% identical to any one of SEQ ID NOS: 61-63. In another example, the FIX coding sequence comprises the sequence set forth in any one of SEQ ID NOS: 61-63. In another example, the FIX coding sequence consists essentially of the sequence set forth in any one of SEQ ID NOS: 61-63. In another example, the FIX coding sequence consists of the sequence set forth in any one of SEQ ID NOS: 61-63. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 61. In another example, the FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the FIX coding sequence is (or comprises a sequence) at least 99%, at least 99.5%, or 100% identical to SEQ ID NO: 61. In another example, the FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the FIX coding sequence comprises the sequence set forth in SEQ ID NO: 61. In another example, the FIX coding sequence consists essentially of the sequence set forth in SEQ ID NO: 61. In another example, the FIX coding sequence consists of the sequence set forth in SEQ ID NO: 61. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

When specific F9 nucleic acid constructs sequences are disclosed herein, they are meant to encompass the sequence disclosed or the reverse complement of the sequence. For example, if a F9 nucleic acid construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. Likewise, when unidirectional construct elements are disclosed herein in a specific 5′ to 3′ order, they are also meant to encompass the reverse complement of the order of those elements. One reason for this is that, in many embodiments disclosed herein, the F9 nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (2) Lysosomal Alpha-Glucosidase (GAA)

Lysosomal alpha-glucosidase (GAA; also known as acid alpha-glucosidase, acid alpha-glucosidase preproprotein, acid maltase, aglucosidase alfa, alpha-1,4-glucosidase, amyloglucosidase, glucoamylase, LYAG) is encoded by GAA. This enzyme is active in lysosomes, where it breaks down glycogen into glucose.

The human GAA gene (NCBI GeneID 2548) encodes a 952 amino acid protein. In the lysosome, human GAA is sequentially processed by proteases to polypeptides of 76-, 19.4-, and 3.9-kDa that remain associated. Further cleavage between R(200) and A(204) inefficiently converts the 76-kDa polypeptide to the mature 70-kDa form with an additional 10.4-kDa polypeptide. GAA maturation increases its affinity for glycogen by 7-10 fold. A signal peptide is encoded by amino acids 1-27, a propeptide encoded by amino acids 28-69, lysosomal alpha-glucosidase after removal of the signal peptide and propeptide is encoded by amino acids 70-952, the 76 kDa lysosomal alpha-glucosidase is encoded by amino acids 123-952, and the 70 kDa lysosomal alpha-glucosidase is encoded by amino acids 204-952.

The GAA expressed from the compositions and methods disclosed herein can be any wild type or variant GAA. In one example, the GAA is a human GAA protein. Human GAA is assigned UniProt reference number P10253. An exemplary amino acid sequence for human GAA is assigned NCBI Accession No. NP_000143.2 and is set forth in SEQ ID NO: 293. An exemplary human GAA mRNA (cDNA) sequence is assigned NCBI Accession No. NM_000152.5 and is set forth in SEQ ID NO: 294. An exemplary human GAA coding sequence is assigned CCDS ID CCDS32760.1 and is set forth in SEQ ID NO: 295. An exemplary mature human GAA amino acid sequence (i.e., the human GAA sequence after removal of the signal peptide and propeptide) starting at amino acid 70 (i.e., GAA 70-952) is set forth in SEQ ID NO: 296. An exemplary coding sequence for GAA 70-952 is set forth in SEQ ID NO: 297.

In some examples, the GAA (e.g., human GAA) is a wild type GAA (e.g., wild type human GAA) sequence or a fragment thereof. For example, the GAA can be a fragment comprising the mature GAA amino acid sequence (i.e., the GAA sequence after removal of the signal peptide and propeptide), a fragment comprising the 77 kDa form of GAA, or a fragment comprising the 70 kDa form of GAA. In a specific example, the GAA can comprise SEQ ID NO: 296 or can be at least 90%, at least 91%, at least 92%, at least 93%, at least 94%, at least 95%, at least 96%, at least 97%, at least 98%, at least 99%, or at least 99.5% identical to SEQ ID NO: 296. In another specific example, the GAA can consist essentially of SEQ ID NO: 296. In another specific example, the GAA can consist of SEQ ID NO: 296.

The GAA coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a GAA coding sequence in a construct disclosed herein has one or more cryptic splice sites mutated or removed. In some embodiments, the nucleotide at position 1095 (or the corresponding position when the GAA coding sequence is aligned with SEQ ID NO: 857) is a “G.” In some embodiments, the nucleotide at position 1098 (or the corresponding position when the GAA coding sequence is aligned with SEQ ID NO: 857) is a “C.” In some embodiments, the nucleotide at position 2343 (or the corresponding position when the GAA coding sequence is aligned with SEQ ID NO: 857) is a “G.” In some embodiments, the nucleotide at position 1095 (or the corresponding position when the GAA coding sequence is aligned with SEQ ID NO: 857) is a “G,” the nucleotide at position 1098 (or the corresponding position when the GAA coding sequence is aligned with SEQ ID NO: 857) is a “C,” and the nucleotide at position 2343 (or the corresponding position when the GAA coding sequence is aligned with SEQ ID NO: 857) is a “G.” In another example, a GAA coding sequence in a construct disclosed herein has all identified cryptic splice sites mutated or removed. In another example, a GAA coding sequence in a construct disclosed herein has one or more CpG dinucleotides removed (i.e., is CpG depleted). In another example, a GAA coding sequence in a construct disclosed herein has all CpG dinucleotides removed (i.e., is fully CpG depleted). In another example, a GAA coding sequence in a construct disclosed herein is codon optimized (e.g., codon optimized for expression in a human or mammal). In a specific example, a GAA coding sequence in a construct disclosed herein has one or more CpG dinucleotides removed (i.e., is CpG depleted) and has one or more cryptic splice sites mutated or removed. In another specific example, a GAA coding sequence in a construct disclosed herein has all CpG dinucleotides removed and has one or more or all identified cryptic splice sites mutated or removed. In another specific example, a GAA coding sequence in a construct disclosed herein has one or more CpG dinucleotides removed (i.e., is CpG depleted) and is codon optimized (e.g., codon optimized for expression in a human or mammal). In another specific example, a GAA coding sequence in a construct disclosed herein has all CpG dinucleotides removed (i.e., is fully CpG depleted) and is codon optimized (e.g., codon optimized for expression in a human or mammal).

Various GAA coding sequences are provided. See, e.g., PCT/US2023/061858 and U.S. Ser. No. 18/163,698, each of which is herein incorporated by reference in its entirety for all purposes. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 297-305 and 326-333. In another example, the GAA coding sequence is (or comprises a sequence) at least 95%, at least 96%, at least 97%, at least 98%, at least 99%, at least 99.5%, or 100% identical to any one of SEQ ID NOS: 297-305 and 326-333. In another example, the GAA coding sequence is (or comprises a sequence) at least 99%, at least 99.5%, or 100% identical to any one of SEQ ID NOS: 297-305 and 326-333. In another example, the GAA coding sequence comprises the sequence set forth in any one of SEQ ID NOS: 297-305 and 326-333. In another example, the GAA coding sequence consists essentially of the sequence set forth in any one of SEQ ID NOS: 297-305 and 326-333. In another example, the GAA coding sequence consists of the sequence set forth in any one of SEQ ID NOS: 297-305 and 326-333. Various GAA coding sequences are provided. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 297-305. In another example, the GAA coding sequence is (or comprises a sequence) at least 95%, at least 96%, at least 97%, at least 98%, at least 99%, at least 99.5%, or 100% identical to any one of SEQ ID NOS: 297-305. In another example, the GAA coding sequence is (or comprises a sequence) at least 99%, at least 99.5%, or 100% identical to any one of SEQ ID NOS: 297-305. In another example, the GAA coding sequence comprises the sequence set forth in any one of SEQ ID NOS: 297-305. In another example, the GAA coding sequence consists essentially of the sequence set forth in any one of SEQ ID NOS: 297-305. In another example, the GAA coding sequence consists of the sequence set forth in any one of SEQ ID NOS: 297-305. In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 299. In another example, the GAA coding sequence is (or comprises a sequence) at least 95%, at least 96%, at least 97%, at least 98%, at least 99%, at least 99.5%, or 100% identical to SEQ ID NO: 299. In another example, the GAA coding sequence is (or comprises a sequence) at least 99%, at least 99.5%, or 100% identical to SEQ ID NO: 299. In another example, the GAA coding sequence comprises the sequence set forth in SEQ ID NO: 299. In another example, the GAA coding sequence consists essentially of the sequence set forth in SEQ ID NO: 299. In another example, the GAA coding sequence consists of the sequence set forth in SEQ ID NO: 299. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296.

Various codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 857, 856, and 299. In another example, the GAA coding sequence is (or comprises a sequence) at least 95%, at least 96%, at least 97%, at least 98%, at least 99%, at least 99.5%, or 100% identical to any one of SEQ ID NOS: 857, 856, and 299. In another example, the GAA coding sequence is (or comprises a sequence) at least 99%, at least 99.5%, or 100% identical to any one of SEQ ID NOS: 857, 856, and 299. In another example, the GAA coding sequence comprises the sequence set forth in any one of SEQ ID NOS: 857, 856, and 299. In another example, the GAA coding sequence consists essentially of the sequence set forth in any one of SEQ ID NOS: 857, 856, and 299. In another example, the GAA coding sequence consists of the sequence set forth in any one of SEQ ID NOS: 857, 856, and 299. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296. In some embodiments, the nucleotide at position 1095 (or the corresponding position when the GAA coding sequence is aligned with SEQ ID NO: 857) is a “G.” In some embodiments, the nucleotide at position 1098 (or the corresponding position when the GAA coding sequence is aligned with SEQ ID NO: 857) is a “C.” In some embodiments, the nucleotide at position 2343 (or the corresponding position when the GAA coding sequence is aligned with SEQ ID NO: 857) is a “G.” In some embodiments, the nucleotide at position 1095 (or the corresponding position when the GAA coding sequence is aligned with SEQ ID NO: 857) is a “G,” the nucleotide at position 1098 (or the corresponding position when the GAA coding sequence is aligned with SEQ ID NO: 857) is a “C,” and the nucleotide at position 2343 (or the corresponding position when the GAA coding sequence is aligned with SEQ ID NO: 857) is a “G.”

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 857. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 857 and encodes a GAA protein (or a GAA protein comprising a sequence) at least 99%, at least 99.5%, or 100% identical to SEQ ID NO: 296. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 857 and encodes a GAA protein comprising the sequence set forth in SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 857. In another example, the GAA coding sequence is (or comprises a sequence) at least 95%, at least 96%, at least 97%, at least 98%, at least 99%, at least 99.5%, or 100% identical to SEQ ID NO: 857 and encodes a GAA protein (or a GAA protein comprising a sequence) at least 99%, at least 99.5%, or 100% identical to SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 857 and encodes a GAA protein comprising the sequence set forth in SEQ ID NO: 296. In another example, the GAA coding sequence is (or comprises a sequence) at least 99%, at least 99.5%, or 100% identical to SEQ ID NO: 857. In another example, the GAA coding sequence is (or comprises a sequence) at least 99%, at least 99.5%, or 100% identical to SEQ ID NO: 857 and encodes a GAA protein (or a GAA protein comprising a sequence) at least 99%, at least 99.5%, or 100% identical to SEQ ID NO: 296. In another example, the GAA coding sequence is (or comprises a sequence) at least 99%, at least 99.5%, or 100% identical to SEQ ID NO: 857 and encodes a GAA protein comprising the sequence set forth in SEQ ID NO: 296. In another example, the GAA coding sequence comprises the sequence set forth in SEQ ID NO: 857. In another example, the GAA coding sequence consists essentially of the sequence set forth in SEQ ID NO: 857. In another example, the GAA coding sequence consists of the sequence set forth in SEQ ID NO: 857. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296. In some embodiments, the nucleotide at position 1095 (or the corresponding position when the GAA coding sequence is aligned with SEQ ID NO: 857) is a “G.” In some embodiments, the nucleotide at position 1098 (or the corresponding position when the GAA coding sequence is aligned with SEQ ID NO: 857) is a “C.” In some embodiments, the nucleotide at position 2343 (or the corresponding position when the GAA coding sequence is aligned with SEQ ID NO: 857) is a “G.” In some embodiments, the nucleotide at position 1095 (or the corresponding position when the GAA coding sequence is aligned with SEQ ID NO: 857) is a “G,” the nucleotide at position 1098 (or the corresponding position when the GAA coding sequence is aligned with SEQ ID NO: 857) is a “C,” and the nucleotide at position 2343 (or the corresponding position when the GAA coding sequence is aligned with SEQ ID NO: 857) is a “G.”

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 856. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 856 and encodes a GAA protein (or a GAA protein comprising a sequence) at least 99%, at least 99.5%, or 100% identical to SEQ ID NO: 296. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 856 and encodes a GAA protein comprising the sequence set forth in SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 856. In another example, the GAA coding sequence is (or comprises a sequence) at least 95%, at least 96%, at least 97%, at least 98%, at least 99%, at least 99.5%, or 100% identical to SEQ ID NO: 856 and encodes a GAA protein (or a GAA protein comprising a sequence) at least 99%, at least 99.5%, or 100% identical to SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 856 and encodes a GAA protein comprising the sequence set forth in SEQ ID NO: 296. In another example, the GAA coding sequence is (or comprises a sequence) at least 99%, at least 99.5%, or 100% identical to SEQ ID NO: 856. In another example, the GAA coding sequence is (or comprises a sequence) at least 99%, at least 99.5%, or 100% identical to SEQ ID NO: 856 and encodes a GAA protein (or a GAA protein comprising a sequence) at least 99%, at least 99.5%, or 100% identical to SEQ ID NO: 296. In another example, the GAA coding sequence is (or comprises a sequence) at least 99%, at least 99.5%, or 100% identical to SEQ ID NO: 856 and encodes a GAA protein comprising the sequence set forth in SEQ ID NO: 296. In another example, the GAA coding sequence comprises the sequence set forth in SEQ ID NO: 856. In another example, the GAA coding sequence consists essentially of the sequence set forth in SEQ ID NO: 856. In another example, the GAA coding sequence consists of the sequence set forth in SEQ ID NO: 856. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296. In some embodiments, the nucleotide at position 1095 (or the corresponding position when the GAA coding sequence is aligned with SEQ ID NO: 857) is a “G.” In some embodiments, the nucleotide at position 1098 (or the corresponding position when the GAA coding sequence is aligned with SEQ ID NO: 857) is a “C.” In some embodiments, the nucleotide at position 2343 (or the corresponding position when the GAA coding sequence is aligned with SEQ ID NO: 857) is a “G.” In some embodiments, the nucleotide at position 1095 (or the corresponding position when the GAA coding sequence is aligned with SEQ ID NO: 857) is a “G,” the nucleotide at position 1098 (or the corresponding position when the GAA coding sequence is aligned with SEQ ID NO: 857) is a “C,” and the nucleotide at position 2343 (or the corresponding position when the GAA coding sequence is aligned with SEQ ID NO: 857) is a “G.”

Various codon optimized GAA coding sequences are provided. The GAA coding sequence can be, for example, CpG-depleted (e.g., fully CpG depleted) and/or codon optimized (e.g., CpG depleted (e.g., fully CpG-depleted) and codon optimized). In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 298-305. In another example, the GAA coding sequence is (or comprises a sequence) at least 95%, at least 96%, at least 97%, at least 98%, at least 99%, at least 99.5%, or 100% identical to any one of SEQ ID NOS: 298-305. In another example, the GAA coding sequence is (or comprises a sequence) at least 99%, at least 99.5%, or 100% identical to any one of SEQ ID NOS: 298-305. In another example, the GAA coding sequence comprises the sequence set forth in any one of SEQ ID NOS: 298-305. In another example, the GAA coding sequence consists essentially of the sequence set forth in any one of SEQ ID NOS: 298-305. In another example, the GAA coding sequence consists of the sequence set forth in any one of SEQ ID NOS: 298-305. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 299. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 299 and encodes a GAA protein (or a GAA protein comprising a sequence) at least 99%, at least 99.5%, or 100% identical to SEQ ID NO: 296. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 299 and encodes a GAA protein comprising the sequence set forth in SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 299. In another example, the GAA coding sequence is (or comprises a sequence) at least 95%, at least 96%, at least 97%, at least 98%, at least 99%, at least 99.5%, or 100% identical to SEQ ID NO: 299 and encodes a GAA protein (or a GAA protein comprising a sequence) at least 99%, at least 99.5%, or 100% identical to SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 299 and encodes a GAA protein comprising the sequence set forth in SEQ ID NO: 296. In another example, the GAA coding sequence is (or comprises a sequence) at least 99%, at least 99.5%, or 100% identical to SEQ ID NO: 299. In another example, the GAA coding sequence is (or comprises a sequence) at least 99%, at least 99.5%, or 100% identical to SEQ ID NO: 299 and encodes a GAA protein (or a GAA protein comprising a sequence) at least 99%, at least 99.5%, or 100% identical to SEQ ID NO: 296. In another example, the GAA coding sequence is (or comprises a sequence) at least 99%, at least 99.5%, or 100% identical to SEQ ID NO: 299 and encodes a GAA protein comprising the sequence set forth in SEQ ID NO: 296. In another example, the GAA coding sequence comprises the sequence set forth in SEQ ID NO: 299. In another example, the GAA coding sequence consists essentially of the sequence set forth in SEQ ID NO: 299. In another example, the GAA coding sequence consists of the sequence set forth in SEQ ID NO: 299. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296.

In one example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 297. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 297 and encodes a GAA protein (or a GAA protein comprising a sequence) at least 99%, at least 99.5%, or 100% identical to SEQ ID NO: 296. In another example, the GAA coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 297 and encodes a GAA protein comprising the sequence set forth in SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 297. In another example, the GAA coding sequence is (or comprises a sequence) at least 95%, at least 96%, at least 97%, at least 98%, at least 99%, at least 99.5%, or 100% identical to SEQ ID NO: 297 and encodes a GAA protein (or a GAA protein comprising a sequence) at least 99%, at least 99.5%, or 100% identical to SEQ ID NO: 296. In another example, the GAA coding sequence is (or comprises a sequence) at least 95%, at least 96%, at least 97%, at least 98%, at least 99%, at least 99.5%, or 100% identical to SEQ ID NO: 297 and encodes a GAA protein comprising the sequence set forth in SEQ ID NO: 296. In another example, the GAA coding sequence is (or comprises a sequence) at least 99%, at least 99.5%, or 100% identical to SEQ ID NO: 297. In another example, the GAA coding sequence is (or comprises a sequence) at least 99%, at least 99.5%, or 100% identical to SEQ ID NO: 297 and encodes a GAA protein (or a GAA protein comprising a sequence) at least 99%, at least 99.5%, or 100% identical to SEQ ID NO: 296. In another example, the GAA coding sequence is (or comprises a sequence) at least 99%, at least 99.5%, or 100% identical to SEQ ID NO: 297 and encodes a GAA protein comprising the sequence set forth in SEQ ID NO: 296. In another example, the GAA coding sequence comprises the sequence set forth in SEQ ID NO: 297. In another example, the GAA coding sequence consists essentially of the sequence set forth in SEQ ID NO: 297. In another example, the GAA coding sequence consists of the sequence set forth in SEQ ID NO: 297. The GAA coding sequence can be, for example, CpG-depleted (e.g., fully CpG-depleted) and/or codon optimized. For example, the GAA coding sequence can be CpG depleted (e.g., fully CpG-depleted) and codon optimized. Optionally, the GAA coding sequence encodes a GAA protein (or a GAA protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence encodes a GAA protein (or a GAA protein comprising a sequence) at least 95%, at least 96%, at least 97%, at least 98%, at least 99%, at least 99.5%, or 100% identical to SEQ ID NO: 296 (and, e.g., retaining the activity of native GAA). Optionally, the GAA coding sequence in the above examples encodes a GAA protein (or a GAA protein comprising a sequence) at least 99%, at least 99.5%, or 100% identical to SEQ ID NO: 296 (and, e.g., retaining the activity of native GAA). Optionally, the GAA coding sequence in the above examples encodes a GAA protein comprising the sequence set forth in SEQ ID NO: 296. Optionally, the GAA coding sequence in the above examples encodes a GAA protein consisting essentially of the sequence set forth in SEQ ID NO: 296. Optionally, the GAA coding sequence in the above examples encodes a GAA protein consisting of the sequence set forth in SEQ ID NO: 296.

When specific GAA or multidomain therapeutic protein nucleic acid constructs sequences are disclosed herein, they are meant to encompass the sequence disclosed or the reverse complement of the sequence. For example, if a GAA or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the GAA or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (3) CD63-Binding Delivery Domain

The multidomain therapeutic proteins disclosed herein can comprise a CD63-binding delivery domain fused to a GAA. See, e.g., PCT/US2023/061858 and U.S. Ser. No. 18/163,698, each of which is herein incorporated by reference in its entirety for all purposes. The CD63-binding domain provides binding to the internalization factor CD63 (UniProt Ref. P08962-1). CD63 (also known as CD63 antigen, granulophysin, lysosomal-associated membrane protein 3, LAMP-3, lysosome integral membrane protein 1, Limp1, melanoma-associated antigen ME491, OMA81H, ocular melanoma-associated antigen, tetraspanin-30, or Tspan-30) is a member of the tetraspanin superfamily of cell surface proteins that span the cell membrane four times. It is encoded by the CD63 gene (also known as MIA1 or TSPAN30). CD63 is expressed in virtually all tissues and is thought to be involved in forming and stabilizing signaling complexes. CD63 localizes to the cell membrane, lysosomal membrane, and late endosomal membrane. CD63 is known to associate with integrins and may be involved in epithelial-mesenchymal transitioning.

In some multidomain therapeutic proteins, the CD63-binding delivery domain is an antibody, an antibody fragment or other antigen-binding protein. In some multidomain therapeutic proteins, the CD63-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain. Examples of CD63-binding delivery domains can be found in WO 2013/138400, WO 2017/007796, WO 2017/190079, WO 2017/100467, WO 2018/226861, WO 2019/157224, and WO 2019/222663, each of which is herein incorporated by reference in its entirety for all purposes.

In a particular multidomain therapeutic protein, the CD63-binding delivery domain is an anti-CD63 scFv. In a specific example, the anti-CD63 scFv can comprise SEQ ID NO: 306 or can be at least 90%, at least 91%, at least 92%, at least 93%, at least 94%, at least 95%, at least 96%, at least 97%, at least 98%, at least 99%, or at least 99.5% identical to SEQ ID NO: 306. In another specific example, the anti-CD63 scFv can consist essentially of SEQ ID NO: 306. In another specific example, the anti-CD63 scFv can consist of SEQ ID NO: 306.

The CD63-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a CD63-binding delivery domain coding sequence in a construct disclosed herein has one or more cryptic splice sites mutated or removed. In another example, a CD63-binding delivery domain coding sequence in a construct disclosed herein has all identified cryptic splice sites mutated or removed. In another example, a CD63-binding delivery domain coding sequence in a construct disclosed herein has one or more CpG dinucleotides removed (i.e., is CpG depleted). In another example, a CD63-binding delivery domain coding sequence in a construct disclosed herein has all CpG dinucleotides removed (i.e., is fully CpG depleted). In another example, a CD63-binding delivery domain coding sequence in a construct disclosed herein is codon optimized (e.g., codon optimized for expression in a human or mammal). In a specific example, a CD63-binding delivery domain coding sequence in a construct disclosed herein has one or more CpG dinucleotides removed (i.e., is CpG depleted) and has one or more cryptic splice sites mutated or removed. In another specific example, a CD63-binding delivery domain coding sequence in a construct disclosed herein has all CpG dinucleotides removed and has one or more or all identified cryptic splice sites mutated or removed. In another specific example, a CD63-binding delivery domain coding sequence in a construct disclosed herein has one or more CpG dinucleotides removed (i.e., is CpG depleted) and is codon optimized (e.g., codon optimized for expression in a human or mammal). In another specific example, a CD63-binding delivery domain coding sequence in a construct disclosed herein has all CpG dinucleotides removed (i.e., is fully CpG depleted) and is codon optimized (e.g., codon optimized for expression in a human or mammal).

Various anti-CD63 scFv coding sequences are provided. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 866, 867, and 309. In another example, the anti-CD63 scFv coding sequence is (or comprises a sequence) at least 95%, at least 96%, at least 97%, at least 98%, at least 99%, at least 99.5%, or 100% identical to any one of SEQ ID NOS: 866, 867, and 309. In another example, the anti-CD63 scFv coding sequence is (or comprises a sequence) at least 99%, at least 99.5%, or 100% identical to any one of SEQ ID NOS: 866, 867, and 309. In another example, the anti-CD63 scFv coding sequence comprises the sequence set forth in any one of SEQ ID NOS: 866, 867, and 309. In another example, the anti-CD63 scFv coding sequence consists essentially of the sequence set forth in any one of SEQ ID NOS: 866, 867, and 309. In another example, the anti-CD63 scFv coding sequence consists of the sequence set forth in any one of SEQ ID NOS: 866, 867, and 309. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 866. In another example, the anti-CD63 scFv coding sequence is (or comprises a sequence) at least 95%, at least 96%, at least 97%, at least 98%, at least 99%, at least 99.5%, or 100% identical to SEQ ID NO: 866. In another example, the anti-CD63 scFv coding sequence is (or comprises a sequence) at least 99%, at least 99.5%, or 100% identical to SEQ ID NO: 866. In another example, the anti-CD63 scFv coding sequence comprises the sequence set forth in SEQ ID NO: 866. In another example, the anti-CD63 scFv coding sequence consists essentially of the sequence set forth in SEQ ID NO: 866. In another example, the anti-CD63 scFv coding sequence consists of the sequence set forth in SEQ ID NO: 866. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306. In some embodiments, the nucleotide at position 3 (or the corresponding position when the anti-CD63 scFv coding sequence is aligned with SEQ ID NO: 866) is an “A.” In some embodiments, the nucleotide at position 132 (or the corresponding position when the anti-CD63 scFv coding sequence is aligned with SEQ ID NO: 866) is an “A.” In some embodiments, the nucleotide at position 273 (or the corresponding position when the anti-CD63 scFv coding sequence is aligned with SEQ ID NO: 866) is a “T.” In some embodiments, the nucleotide at position 3 (or the corresponding position when the anti-CD63 scFv coding sequence is aligned with SEQ ID NO: 866) is an “A,” the nucleotide at position 132 (or the corresponding position when the anti-CD63 scFv coding sequence is aligned with SEQ ID NO: 866) is an “A,” and the nucleotide at position 273 (or the corresponding position when the anti-CD63 scFv coding sequence is aligned with SEQ ID NO: 866) is a “T.”

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 866. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 866 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 866 and encodes an anti-CD63 scFv protein comprising the sequence set forth in SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 866. In another example, the anti-CD63 scFv coding sequence is (or comprises a sequence) at least 95%, at least 96%, at least 97%, at least 98%, at least 99%, at least 99.5%, or 100% identical to SEQ ID NO: 866 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 866 and encodes an anti-CD63 scFv protein comprising the sequence set forth in SEQ ID NO: 306. In another example, the anti-CD63 scFv coding sequence is (or comprises a sequence) at least 99%, at least 99.5%, or 100% identical to SEQ ID NO: 866. In another example, the anti-CD63 scFv coding sequence is (or comprises a sequence) at least 99%, at least 99.5%, or 100% identical to SEQ ID NO: 866 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 99%, at least 99.5%, or 100% identical to SEQ ID NO: 866 and encodes an anti-CD63 scFv protein comprising the sequence set forth in SEQ ID NO: 306. In another example, the anti-CD63 scFv coding sequence comprises the sequence set forth in SEQ ID NO: 866. In another example, the anti-CD63 scFv coding sequence consists essentially of the sequence set forth in SEQ ID NO: 866. In another example, the anti-CD63 scFv coding sequence consists of the sequence set forth in SEQ ID NO: 866. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306. In some embodiments, the nucleotide at position 3 (or the corresponding position when the anti-CD63 scFv coding sequence is aligned with SEQ ID NO: 866) is an “A.” In some embodiments, the nucleotide at position 132 (or the corresponding position when the anti-CD63 scFv coding sequence is aligned with SEQ ID NO: 866) is an “A.” In some embodiments, the nucleotide at position 273 (or the corresponding position when the anti-CD63 scFv coding sequence is aligned with SEQ ID NO: 866) is a “T.” In some embodiments, the nucleotide at position 3 (or the corresponding position when the anti-CD63 scFv coding sequence is aligned with SEQ ID NO: 866) is an “A,” the nucleotide at position 132 (or the corresponding position when the anti-CD63 scFv coding sequence is aligned with SEQ ID NO: 866) is an “A,” and the nucleotide at position 273 (or the corresponding position when the anti-CD63 scFv coding sequence is aligned with SEQ ID NO: 866) is a “T.”

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 867. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 867 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 867 and encodes an anti-CD63 scFv protein comprising the sequence set forth in SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 867. In another example, the anti-CD63 scFv coding sequence is (or comprises a sequence) at least 95%, at least 96%, at least 97%, at least 98%, at least 99%, at least 99.5%, or 100% identical to SEQ ID NO: 867 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 867 and encodes an anti-CD63 scFv protein comprising the sequence set forth in SEQ ID NO: 306. In another example, the anti-CD63 scFv coding sequence is (or comprises a sequence) at least 99%, at least 99.5%, or 100% identical to SEQ ID NO: 867. In another example, the anti-CD63 scFv coding sequence is (or comprises a sequence) at least 99%, at least 99.5%, or 100% identical to SEQ ID NO: 867 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 99%, at least 99.5%, or 100% identical to SEQ ID NO: 867 and encodes an anti-CD63 scFv protein comprising the sequence set forth in SEQ ID NO: 306. In another example, the anti-CD63 scFv coding sequence comprises the sequence set forth in SEQ ID NO: 867. In another example, the anti-CD63 scFv coding sequence consists essentially of the sequence set forth in SEQ ID NO: 867. In another example, the anti-CD63 scFv coding sequence consists of the sequence set forth in SEQ ID NO: 867. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306. In some embodiments, the nucleotide at position 3 (or the corresponding position when the anti-CD63 scFv coding sequence is aligned with SEQ ID NO: 866) is an “A.” In some embodiments, the nucleotide at position 132 (or the corresponding position when the anti-CD63 scFv coding sequence is aligned with SEQ ID NO: 866) is an “A.” In some embodiments, the nucleotide at position 273 (or the corresponding position when the anti-CD63 scFv coding sequence is aligned with SEQ ID NO: 866) is a “T.” In some embodiments, the nucleotide at position 3 (or the corresponding position when the anti-CD63 scFv coding sequence is aligned with SEQ ID NO: 866) is an “A,” the nucleotide at position 132 (or the corresponding position when the anti-CD63 scFv coding sequence is aligned with SEQ ID NO: 866) is an “A,” and the nucleotide at position 273 (or the corresponding position when the anti-CD63 scFv coding sequence is aligned with SEQ ID NO: 866) is a “T.”

Various anti-CD63 scFv coding sequences are provided. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 307-315. In another example, the anti-CD63 scFv coding sequence is (or comprises a sequence) at least 95%, at least 96%, at least 97%, at least 98%, at least 99%, at least 99.5%, or 100% identical to any one of SEQ ID NOS: 307-315. In another example, the anti-CD63 scFv coding sequence is (or comprises a sequence) at least 99%, at least 99.5%, or 100% identical to any one of SEQ ID NOS: 307-315. In another example, the anti-CD63 scFv coding sequence comprises the sequence set forth in any one of SEQ ID NOS: 307-315. In another example, the anti-CD63 scFv coding sequence consists essentially of the sequence set forth in any one of SEQ ID NOS: 307-315. In another example, the anti-CD63 scFv coding sequence consists of the sequence set forth in any one of SEQ ID NOS: 307-315. In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 309. In another example, the anti-CD63 scFv coding sequence is (or comprises a sequence) at least 95%, at least 96%, at least 97%, at least 98%, at least 99%, at least 99.5%, or 100% identical to SEQ ID NO: 309. In another example, the anti-CD63 scFv coding sequence is (or comprises a sequence) at least 99%, at least 99.5%, or 100% identical to SEQ ID NO: 309. In another example, the anti-CD63 scFv coding sequence comprises the sequence set forth in SEQ ID NO: 309. In another example, the anti-CD63 scFv coding sequence consists essentially of the sequence set forth in SEQ ID NO: 309. In another example, the anti-CD63 scFv coding sequence consists of the sequence set forth in SEQ ID NO: 309. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306.

Various codon optimized anti-CD63 scFv coding sequences are provided. The anti-CD63 scFv coding sequence can be, for example, CpG-depleted (e.g., fully CpG depleted) and/or codon optimized (e.g., CpG depleted (e.g., fully CpG-depleted) and codon optimized). In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 308-315. In another example, the anti-CD63 scFv coding sequence is (or comprises a sequence) at least 95%, at least 96%, at least 97%, at least 98%, at least 99%, at least 99.5%, or 100% identical to any one of SEQ ID NOS: 308-315. In another example, the anti-CD63 scFv coding sequence is (or comprises a sequence) at least 99%, at least 99.5%, or 100% identical to any one of SEQ ID NOS: 308-315. In another example, the anti-CD63 scFv coding sequence comprises the sequence set forth in any one of SEQ ID NOS: 308-315. In another example, the anti-CD63 scFv coding sequence consists essentially of the sequence set forth in any one of SEQ ID NOS: 308-315. In another example, the anti-CD63 scFv coding sequence consists of the sequence set forth in any one of SEQ ID NOS: 308-315. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 309. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 309 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 309 and encodes an anti-CD63 scFv protein comprising the sequence set forth in SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 309. In another example, the anti-CD63 scFv coding sequence is (or comprises a sequence) at least 95%, at least 96%, at least 97%, at least 98%, at least 99%, at least 99.5%, or 100% identical to SEQ ID NO: 309 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 309 and encodes an anti-CD63 scFv protein comprising the sequence set forth in SEQ ID NO: 306. In another example, the anti-CD63 scFv coding sequence is (or comprises a sequence) at least 99%, at least 99.5%, or 100% identical to SEQ ID NO: 309. In another example, the anti-CD63 scFv coding sequence is (or comprises a sequence) at least 99%, at least 99.5%, or 100% identical to SEQ ID NO: 309 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 99%, at least 99.5%, or 100% identical to SEQ ID NO: 309 and encodes an anti-CD63 scFv protein comprising the sequence set forth in SEQ ID NO: 306. In another example, the anti-CD63 scFv coding sequence comprises the sequence set forth in SEQ ID NO: 309. In another example, the anti-CD63 scFv coding sequence consists essentially of the sequence set forth in SEQ ID NO: 309. In another example, the anti-CD63 scFv coding sequence consists of the sequence set forth in SEQ ID NO: 309. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306.

In one example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 307. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 307 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 307 and encodes an anti-CD63 scFv protein comprising the sequence set forth in SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 307. In another example, the anti-CD63 scFv coding sequence is (or comprises a sequence) at least 95%, at least 96%, at least 97%, at least 98%, at least 99%, at least 99.5%, or 100% identical to SEQ ID NO: 307 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 95%, at least 96%, at least 97%, at least 98%, at least 99%, at least 99.5%, or 100% identical to SEQ ID NO: 307 and encodes an anti-CD63 scFv protein comprising the sequence set forth in SEQ ID NO: 306. In another example, the anti-CD63 scFv coding sequence is (or comprises a sequence) at least 99%, at least 99.5%, or 100% identical to SEQ ID NO: 307. In another example, the anti-CD63 scFv coding sequence is (or comprises a sequence) at least 99%, at least 99.5%, or 100% identical to SEQ ID NO: 307 and encodes an anti-CD63 scFv protein (or an anti-CD63 scFv protein comprising a sequence) at least 99%, at least 99.5%, or 100% identical to SEQ ID NO: 306. In another example, the anti-CD63 scFv coding sequence is (or comprises a sequence) at least 99%, at least 99.5%, or 100% identical to SEQ ID NO: 307 and encodes an anti-CD63 scFv protein comprising the sequence set forth in SEQ ID NO: 306. In another example, the anti-CD63 scFv coding sequence comprises the sequence set forth in SEQ ID NO: 307. In another example, the anti-CD63 scFv coding sequence consists essentially of the sequence set forth in SEQ ID NO: 307. In another example, the anti-CD63 scFv coding sequence consists of the sequence set forth in SEQ ID NO: 307. The anti-CD63 scFv coding sequence can be, for example, CpG-depleted (e.g., fully CpG-depleted) and/or codon optimized. For example, the anti-CD63 scFv coding sequence can be CpG depleted (e.g., fully CpG-depleted) and codon optimized. Optionally, the anti-CD63 scFv coding sequence encodes an anti-CD63 scFv protein (or an anti-CD63 scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence encodes an anti-CD63 scFv protein (or an anti-CD63 scFv protein comprising a sequence) at least 95%, at least 96%, at least 97%, at least 98%, at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein (or an anti-CD63 scFv protein comprising a sequence) at least 99%, at least 99.5%, or 100% identical to SEQ ID NO: 306 (and, e.g., retaining CD63-binding activity). Optionally, the anti-CD63 scFv coding sequence in the above examples encodes an anti-CD63 scFv protein comprising the sequence set forth in SEQ ID NO: 306. Optionally, the anti-CD63 scFv coding sequence in the above examples encodes an anti-CD63 scFv protein consisting essentially of the sequence set forth in SEQ ID NO: 306. Optionally, the anti-CD63 scFv coding sequence in the above examples encodes an anti-CD63 scFv protein consisting of the sequence set forth in SEQ ID NO: 306.

When specific anti-CD63 scFv or multidomain therapeutic protein nucleic acid constructs sequences are disclosed herein, they are meant to encompass the sequence disclosed or the reverse complement of the sequence. For example, if an anti-CD63 scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-CD63 scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (4) TfR-Binding Delivery Domain

The multidomain therapeutic proteins disclosed herein can comprise a TfR-binding delivery domain fused to a GAA. See, e.g., PCT/US2023/061858 and U.S. Ser. No. 18/163,698, each of which is herein incorporated by reference in its entirety for all purposes. The TfR-binding domain provides binding to the internalization factor transferrin receptor protein 1 (TfR; UniProt Ref. P02786). TfR (also known as TR, TfR1, and Trfr) is encoded by the TFRC gene. TfR is expressed in muscle and on brain endothelial cells. Transcytosis of TfR in these cells enables blood-brain-barrier crossing. In some embodiments, the multidomain therapeutic proteins comprising a TfR-binding delivery domain (e.g., scFv) fused to a GAA do not alter transferrin uptake. In some embodiments, the multidomain therapeutic proteins comprising a TfR-binding delivery domain (e.g., scFv) fused to a GAA do not alter iron homeostasis. In some embodiments, the multidomain therapeutic proteins comprising a TfR-binding delivery domain (e.g., scFv) fused to a GAA do not alter transferrin uptake or iron homeostasis.

Transferrin receptor 1 (TfR) is a membrane receptor involved in the control of iron supply to the cell through the binding of transferrin, the major iron-carrier protein. Transferrin receptor 1 is expressed from the TFRC gene. Transferrin receptor 1 may be referred to, herein, at TFRC. This receptor plays a key role in the control of cell proliferation because iron is essential for sustaining ribonucleotide reductase activity, and is the only enzyme that catalyzes the conversion of ribonucleotides to deoxyribonucleotides. Preferably, the TfR is human TfR (hTfR). See e.g., Accession numbers NP_001121620.1; BAD92491.1; and NP_001300894.1; and e!Ensembl entry: ENSG00000072274. The human transferrin receptor 1 is expressed in several tissues, including but not limited to: cerebral cortex; cerebellum; hippocampus; caudate; parathyroid gland; adrenal gland; bronchus; lung; oral mucosa; esophagus; stomach; duodenum; small intestine; colon; rectum; liver; gallbladder; pancreas; kidney; urinary bladder; testis; epididymis; prostate; vagina; ovary; fallopian tube; endometrium; cervix; placenta; breast; heart muscle; smooth muscle; soft tissue; skin; appendix; lymph node; tonsil; and bone marrow. A related transferrin receptor is transferrin receptor 2 (TfR2). Human transferrin receptor 2 bears about 45% sequence identity to human transferrin receptor 1. Trinder & Baker, Transferrin receptor 2: a new molecule in iron metabolism. Int J Biochem Cell Biol. 2003 March; 35(3):292-6. Unless otherwise stated, transferrin receptor as used herein generally refers to transferrin receptor 1 (e.g., human transferrin receptor 1).

Human Transferrin (Tf) is a single chain, 80 kDa member of the anion-binding superfamily of proteins. Transferrin is a 698 amino acid precursor that is divided into a 19 aa signal sequence plus a 679 aa mature segment that typically contains 19 intrachain disulfide bonds. The N- and C-terminal flanking regions (or domains) bind ferric iron through the interaction of an obligate anion (e.g., bicarbonate) and four amino acids (His, Asp, and two Tyr). Apotransferrin (or iron-free) will initially bind one atom of iron at the C-terminus, and this is followed by subsequent iron binding by the N-terminus to form holotransferrin (diferric Tf, Holo-Tf). Through its C-terminal iron-binding domain, holotransferrin will interact with the TfR on the surface of cells where it is internalized into acidified endosomes. Iron dissociates from the Tf molecule within these endosomes, and is transported into the cytosol as ferrous iron. In addition to TfR, transferrin is reported to bind to cubulin, IGFBP3, microbial iron-binding proteins and liver-specific TfR2.

The blood-brain barrier (BBB) is located within the microvasculature of the brain, and it regulates passage of molecules from the blood to the brain. Burkhart et al., Accessing targeted nanoparticles to the brain: the vascular route. Curr Med Chem. 2014; 21(36):4092-9. The transcellular passage through the brain capillary endothelial cells can take place via 1) cell entry by leukocytes; 2) carrier-mediated influx of e.g., glucose by glucose transporter 1 (GLUT-1), amino acids by e.g., the L-type amino acid transporter 1 (LAT-1) and small peptides by e.g., organic anion-transporting peptide-B (OATP-B); 3) paracellular passage of small hydrophobic molecules; 4) adsorption-mediated transcytosis of e.g., albumin and cationized molecules; 5) passive diffusion of lipid soluble, non-polar solutes, including CO₂ and O₂; and 5) receptor-mediated transcytosis of, e.g., insulin by the insulin receptor and Tf by the TfR. Johnsen et al., Targeting the transferrin receptor for brain drug delivery, Prog Neurobiol. 2019 October; 181:101665.

For example, anti-TfR:GAA fusion proteins exhibiting high affinity to the transferrin receptor and superior blood-brain barrier crossing are provided. Surprisingly, fusions exhibiting high binding affinity to TfR crossed the blood-brain barrier more efficiently than that of low affinity binders. The fusions of the present invention have an ability to efficiently deliver GAA to the brain and, thus, are an effective treatment of glycogen storage diseases such as Pompe Disease.

Provided herein are antigen-binding proteins, such as antibodies, antigen-binding fragments thereof, such as Fabs and scFvs, that bind specifically to the transferrin receptor, preferably the human transferrin receptor 1 (anti-hTfR). For example, in an embodiment, the anti-hTfR is in the form of a fusion protein. The fusion protein includes the anti-hTfR antigen-binding protein fused to GAA. The anti-hTfRs efficiently cross the blood-brain barrier (BBB) and can, thereby, deliver the fused GAA to the brain.

An antigen-binding protein that specifically binds to transferrin receptor and fusions thereof, for example, a tag such as His₆ and/or myc (e.g., human transferrin receptor (e.g., REGN2431) or monkey transferrin receptor (e.g., REGN2054)) binds at about 25° C., e.g., in a surface plasmon resonance assay, with a K_{D} of about 20 nM or a higher affinity. Such an antigen-binding protein may be referred to as “anti-TfR.”

In an embodiment, an anti-hTfR scFv:GAA fusion protein includes an scFv comprising the arrangement of variable regions as follows LCVR-HCVR or HCVR-LCVR, wherein the HCVR and LCVR are optionally connected by a linker and the scFv is connected, optionally by a linker, to GAA (e.g., LCVR-(Gly₄Ser)₃-HCVR-(Gly₄Ser)₂)-GAA; or LCVR-(Gly₄Ser)₃-HCVR-(Gly₄Ser)₂)-GAA) (Gly₄Ser=SEQ ID NO: 718)). In one example, the linker between the HCVR and LCVR comprises, consists essentially of, or consists of three such repeats (SEQ ID NO: 828). For example, the coding sequence for the linker can comprise, consist essentially of, or consist of any one of SEQ ID NOS: 830-834 and 854. In another example, the linker between the HCVR and LCVR comprises, consists essentially of, or consists of two such repeats (SEQ ID NO: 829). For example, the coding sequence for the linker can comprise, consist essentially of, or consist of any one of SEQ ID NOS: 835-841. In another example, the linker between the HCVR and LCVR comprises, consists essentially of, or consists of one such repeat (SEQ ID NO: 718). For example, the coding sequence for the linker can comprise, consist essentially of, or consist of SEQ ID NO: 842 or 855. In one example, the linker between the scFv and GAA comprises, consists essentially of, or consists of three such repeats (SEQ ID NO: 828). For example, the coding sequence for the linker can comprise, consist essentially of, or consist of any one of SEQ ID NOS: 830-834 and 854. In another example, the linker between the scFv and GAA comprises, consists essentially of, or consists of two such repeats (SEQ ID NO: 829). For example, the coding sequence for the linker can comprise, consist essentially of, or consist of any one of SEQ ID NOS: 835-841. In another example, the linker between the scFv and GAA comprises, consists essentially of, or consists of one such repeat (SEQ ID NO: 718). For example, the coding sequence for the linker can comprise, consist essentially of, or consist of SEQ ID NO: 842 or 855.

An anti-hTfR:GAA optionally comprises a signal peptide, connected to the antigen-binding protein that binds specifically to transferrin receptor (TfR), preferably, human transferrin receptor (hTfR) which is fused (optionally by a linker) to GAA. In an embodiment, the signal peptide is the mROR signal sequence (e.g., mROR signal sequence-LCVR-(Gly₄Ser)₃-HCVR-(Gly₄Ser)₂)-GAA; or LCVR-(Gly₄Ser)₃-HCVR-(Gly₄Ser)₂)-GAA) (Gly₄Ser=SEQ ID NO: 718)). The term “fused” or “tethered” with regard to fused polypeptides refers to polypeptides joined directly or indirectly (e.g., via a linker or other polypeptide).

In an embodiment of the invention, the assignment of amino acids to each framework or CDR domain in an immunoglobulin is in accordance with the definitions of Sequences of Proteins of Immunological Interest, Kabat et al.; National Institutes of Health, Bethesda, Md.; 5^{th} ed.; NIH Publ. No. 91-3242 (1991); Kabat (1978) Adv. Prot. Chem. 32:1-75; Kabat et al., (1977) J. Biol. Chem. 252:6609-6616; Chothia, et al., (1987) J Mol. Biol. 196:901-917 or Chothia, et A, (1989) Nature 342: 878-883. Thus, included are antibodies and antigen-binding fragments including the CDRs of a V_{H} and the CDRs of a V_{L}, which V_{H} and V_{L} comprise amino acid sequences as set forth herein (see, e.g., sequences of Table 3, or variants thereof), wherein the CDRs are as defined according to Kabat and/or Chothia.

TABLE 3
Domains in Anti-hTfR Antibodies, Antigen-binding Fragments (e.g.,
Fabs) or scFv Molecules in Fusion Proteins and SEQ ID NOs.

| | anti-hTfR | HC- | HC- | | | | LC- | LC- | | | |
|---|---|---|---|---|---|---|---|---|---|---|---|
| # | Molecule | VRNT | VRAA | HCDR1 | HCDR2 | HCDR3 | VRNT | VRAA | LCDR1 | LCDR2 | LCDR3 |
| 1 | 31874B | 334 | 335 | 336 | 337 | 338 | 339 | 340 | 341 | 342 | 343 |
| 2 | 31863B | 344 | 345 | 346 | 347 | 348 | 349 | 350 | 351 | 352 | 353 |
| 3 | 69348 | 354 | 355 | 356 | 357 | 358 | 359 | 360 | 361 | 362 | 363 |
| 4 | 69340 | 364 | 365 | 366 | 367 | 368 | 369 | 370 | 371 | 372 | 373 |
| 5 | 69331 | 374 | 375 | 376 | 377 | 378 | 379 | 380 | 381 | 382 | 383 |
| 6 | 69332 | 384 | 385 | 386 | 387 | 388 | 389 | 390 | 391 | 392 | 393 |
| 7 | 69326 | 394 | 395 | 396 | 397 | 398 | 399 | 400 | 401 | 402 | 403 |
| 8 | 69329 | 404 | 405 | 406 | 407 | 408 | 409 | 410 | 411 | 412 | 413 |
| 9 | 69323 | 414 | 415 | 416 | 417 | 418 | 419 | 420 | 421 | 422 | 423 |
| 10 | 69305 | 424 | 425 | 426 | 427 | 428 | 429 | 430 | 431 | 432 | 433 |
| 11 | 69307 | 434 | 435 | 436 | 437 | 438 | 439 | 440 | 441 | 442 | 443 |
| 12 | 12795B | 444 | 445 | 446 | 447 | 448 | 449 | 450 | 451 | 452 | 453 |
| 13 | 12798B | 454 | 455 | 456 | 457 | 458 | 459 | 460 | 461 | 462 | 463 |
| 14 | 12799B | 464 | 465 | 466 | 467 | 468 | 469 | 470 | 471 | 472 | 473 |
| 15 | 12801B | 474 | 475 | 476 | 477 | 478 | 479 | 480 | 481 | 482 | 483 |
| 16 | 12802B | 484 | 485 | 486 | 487 | 488 | 489 | 490 | 491 | 492 | 493 |
| 17 | 12808B | 494 | 495 | 496 | 497 | 498 | 499 | 500 | 501 | 502 | 503 |
| 18 | 12812B | 504 | 505 | 506 | 507 | 508 | 509 | 510 | 511 | 512 | 513 |
| 19 | 12816B | 514 | 515 | 516 | 517 | 518 | 519 | 520 | 521 | 522 | 523 |
| 20 | 12833B | 524 | 525 | 526 | 527 | 528 | 529 | 530 | 531 | 532 | 533 |
| 21 | 12834B | 534 | 535 | 536 | 537 | 538 | 539 | 540 | 541 | 542 | 543 |
| 22 | 12835B | 544 | 545 | 546 | 547 | 548 | 549 | 550 | 551 | 552 | 553 |
| 23 | 12847B | 554 | 555 | 556 | 557 | 558 | 559 | 560 | 561 | 562 | 563 |
| 24 | 12848B | 564 | 565 | 566 | 567 | 568 | 569 | 570 | 571 | 572 | 573 |
| 25 | 12843B | 574 | 575 | 576 | 577 | 578 | 579 | 580 | 581 | 582 | 583 |
| 26 | 12844B | 584 | 585 | 586 | 587 | 588 | 589 | 590 | 591 | 592 | 593 |
| 27 | 12845B | 594 | 595 | 596 | 597 | 598 | 599 | 600 | 601 | 602 | 603 |
| 28 | 12839B | 604 | 605 | 606 | 607 | 608 | 609 | 610 | 611 | 612 | 613 |
| 29 | 12841B | 614 | 615 | 616 | 617 | 618 | 619 | 620 | 621 | 622 | 623 |
| 30 | 12850B | 624 | 625 | 626 | 627 | 628 | 629 | 630 | 631 | 632 | 633 |
| 31 | 69261 | 634 | 635 | 636 | 637 | 638 | 639 | 640 | 641 | 642 | 643 |
| 32 | 69263 | 644 | 645 | 646 | 647 | 648 | 649 | 650 | 651 | 652 | 653 |

31874B
HCVR (V_{H}) Nucleotide Sequence
(SEQ ID NO: 334)
GAGGTGCAGCTGGTGGAGTCTGGGGGAGGCTTGGTACAGCCTGGGGGGTCCCTGA
GACTCTCCTGTGCAGCCTCTGGATTCGCCTTTAGCAGCTATGCCATGACCTGGGT
CCGACAGGCTCCAGGGAAGGGGCTGGAGTGGGTCTCAGTTATCAGTGGTACTGGT
GGTAGTACATACTACGCAGACTCCGTGAAGGGCCGGTTCACCATCTCCAGAGACA
ATTCCAAGAACACGCTGTATCTACAAATGAACAGCCTGAGAGCCGAGGACACGGC
CGTATATTACTGTGCGAAAGGGGGAGCAGCTCGTAGAATGGAATACTTCCAGTAC
TGGGGCCAGGGCACCCTGGTCACCGTCTCCTCA
HCVR (V_{H}) Amino Acid Sequence
(SEQ ID NO: 335)
EVQLVESGGGLVQPGGSLRLSCAASGFAFSSYAMTWVRQAPGKGLEWVSVISGTG
GSTYYADSVKGRFTISRDNSKNTLYLQMNSLRAEDTAVYYCAKGGAARRMEYFQY
WGQGTLVTVSS
HCDR1:
(SEQ ID NO: 336)
GFAFSSYA
HCDR2:
(SEQ ID NO: 337)
ISGTGGST
HCDR3:
(SEQ ID NO: 338)
AKGGAARRMEYFQY
LCVR (V_{L}) Nucleotide Sequence
(SEQ ID NO: 339)
GACATCCAGATGACCCAGTCTCCATCCTCCCTGTCTGCATCTGTAGGAGACAGAG
TCACCATCACTTGCCGGGCGAGTCAGGGCATTAGCAATTATTTAGCCTGGTATCA
GCAGAAACCAGGGAAAGTTCCTAACCTCCTTATCTATGCTGCATCCACTTTGCAA
TCAGGGGTCCCATCTCGATTCAGTGGCAGTGGATCTGGGACAGATTTCACTCTCA
CCATCAGCAGCCTGCAGCCTGAAGATGTTGCAACTTATTACTGTCAAAAGTATAA
CAGTGCCCCTCTCACTTTCGGCGGAGGGACCAAGGTGGAGATCAAA
LCVR (V_{L}) Amino Acid Sequence
(SEQ ID NO: 340)
DIQMTQSPSSLSASVGDRVTITCRASQGISNYLAWYQQKPGKVPNLLIYAASTLQ
SGVPSRFSGSGSGTDFTLTISSLQPEDVATYYCQKYNSAPLTFGGGTKVEIK
LCDR1:
(SEQ ID NO: 341)
QGISNY
LCDR2:
(SEQ ID NO: 342)
AAS
LCDR3:
(SEQ ID NO: 343)
QKYNSAPLT
31863B
HCVR (V_{H}) Nucleotide Sequence
(SEQ ID NO: 344)
GAGGTGCAGCTGGTGGAGTCTGGGGGAGGCTTGGTACAGCCTGGGGGGTCCCTGA
GACTCTCCTGTGCAGCCTCTGGATTCACCTTTAACAGCTATGCCATGACCTGGGT
CCGCCAGGCTCCAGGGAAGGGGCTGGAGTGGGTCTCATTTATTGGTGGTAGTACT
GGTAACACATACTACGCAGGCTCCGTGAAGGGCCGGTTCACCATCTCCAGCGACA
ATTCCAAGAAGACGCTGTATCTGCAAATGAACAGCCTGAGAGCCGAGGACACGGC
CGTATATTACTGTGCGAAAGGGGGAGCAGCTCGTAGAATGGAATACTTCCAGCAC
TGGGGCCAGGGCACCCTGGTCACCGTCTCCTCA
HCVR (V_{H}) Amino Acid Sequence
(SEQ ID NO: 345)
EVQLVESGGGLVQPGGSLRLSCAASGFTFNSYAMTWVRQAPGKGLEWVSFIGGST
GNTYYAGSVKGRFTISSDNSKKTLYLQMNSLRAEDTAVYYCAKGGAARRMEYFQH
WGQGTLVTVSS
HCDR1:
(SEQ ID NO: 346)
GFTFNSYA
HCDR2:
(SEQ ID NO: 347)
IGGSTGNT
HCDR3:
(SEQ ID NO: 348)
AKGGAARRMEYFQH
LCVR (V_{L}) Nucleotide Sequence
(SEQ ID NO: 349)
GACATCCAGATGACCCAGTCTCCATCCTCCCTGTCTGCATCTATAGGAGACAGAG
TCACCATCACTTGCCGGGCGAGTCAGGGCATTAGCAATTATTTAGCCTGGTATCA
ACAGAAACCAGGGAAAGTTCCTAAGCTCCTGATCTATGCTGCATCCACTTTGCAA
TCAGGGGTCCCATCTCGGTTCAGTGGCAGTGGATCTGGGACAGATTTCACTCTCA
CCATCAGCAGCCTGCAGCCTGAAGATGTTGCAACTTATTACTGTCAAAACCATAA
CAGTGTCCCTCTCACTTTCGGCGGAGGGACCAAGGTGGAGATCAAA
LCVR (V_{L}) Amino Acid Sequence
(SEQ ID NO: 350)
DIQMTQSPSSLSASIGDRVTITCRASQGISNYLAWYQQKPGKVPKLLIYAASTLQ
SGVPSRFSGSGSGTDFTLTISSLQPEDVATYYCQNHNSVPLTFGGGTKVEIK
LCDR1:
(SEQ ID NO: 351)
QGISNY
LCDR2:
(SEQ ID NO: 352)
AAS
LCDR3:
(SEQ ID NO: 353)
QNHNSVPLT
69348
HCVR (V_{H}) Nucleotide Sequence
(SEQ ID NO: 354)
CAGGTGCAGCTGGTGGAGTCTGGGGGAGGCGTGGTCCAGCCTGGGAGGTCCCTGA
GACTCTCCTGTGCAGCGTCTGGATTCACCTTCACTACCTATGGCATGCACTGGGT
CCGCCAGGCTCCAGGCAAGGGGCTGGAGTGGGTGGCTGTTATATGGTATGATGGA
AGTAATAAATATTATGGAGACTCCGTGAAGGGCCGATTCACCATCTCCAGAGACA
ATTCCAAGAACACACTGTATCTGCAAATGAACAGCCTGAGAGTCGACGACACGGC
TGTTTATTACTGTACGAGAACCCATGGCTATACCAGGTCGTCGGACGGTTTTGAC
TACTGGGGCCAGGGAACCCTGGTCACCGTCTCCTCA
HCVR (V_{H}) Amino Acid Sequence
(SEQ ID NO: 355)
QVQLVESGGGVVQPGRSLRLSCAASGFTFTTYGMHWVRQAPGKGLEWVAVIWYDG
SNKYYGDSVKGRFTISRDNSKNTLYLQMNSLRVDDTAVYYCTRTHGYTRSSDGFD
YWGQGTLVTVSS
HCDR1:
(SEQ ID NO: 356)
GFTFTTYG
HCDR2:
(SEQ ID NO: 357)
IWYDGSNK
HCDR3:
(SEQ ID NO: 358)
TRTHGYTRSSDGFDY
LCVR (V_{L}) Nucleotide Sequence
(SEQ ID NO: 359)
GACATCCAGATGACCCAGTCTCCATCCTCCCTGTCTGCATCTGTAGGAGACAGAG
TCACCATCACTTGCCGGGCAAGTCAGAGCATTAGAAATGTTTTAGGCTGGTTTCA
GCAGAAACCAGGGAAAGCCCCTCAGCGCCTGATCTATGCTGCATCCAGTTTGCAA
AGTGGGGTCCCATCAAGGTTCAGCGGCAGTGGATCTGGGACAGAATTCACTCTCA
CAATCAGCAGCCTACAGCCTGAAGATTTTGCAACTTATTACTGTCTACAGCATAA
TTTTTACCCGCTCACTTTCGGCGGAGGGACCAAGGTGGAGATCAAA
LCVR (V_{L}) Amino Acid Sequence
(SEQ ID NO: 360)
DIQMTQSPSSLSASVGDRVTITCRASQSIRNVLGWFQQKPGKAPQRLIYAASSLQ
SGVPSRFSGSGSGTEFTLTISSLQPEDFATYYCLQHNFYPLTFGGGTKVEIK
LCDR1:
(SEQ ID NO: 361)
QSIRNV
LCDR2:
(SEQ ID NO: 362)
AAS
LCDR3:
(SEQ ID NO: 363)
LOHNFYPLT
69340
HCVR (V_{H}) Nucleotide Sequence
(SEQ ID NO: 364)
GAAGTGCAGCTGGTGGAGTCTGGGGGAGGCTTGGTACAGCCTGGCAGGTCCCTGA
GACTCTCCTGTGCAGCCTCTGGATTCACCTTTGATGATAAAGCCATGCACTGGGT
CCGGCAAGTTCCAGGGAAGGGCCTGGAATGGATCTCAGGTATTAGTTGGAATAGT
GGTACTATAGGCTATGCGGACTCTGTGAAGGGCCGATTCATCATCTCCAGAGACA
ACGCCAAGAACTCCCTGTATCTACAAATGAACAGTCTGAGAGCTGAGGACACGGC
CTTGTATTACTGCGCAAAAGATGGAGATACCAGTGGCTGGTACTGGTACGGTTTG
GACGTCTGGGGCCAAGGGACCACGGTCACCGTCTCCTCA
HCVR (V_{H}) Amino Acid Sequence
(SEQ ID NO: 365)
EVQLVESGGGLVQPGRSLRLSCAASGFTFDDKAMHWVRQVPGKGLEWISGISWNS
GTIGYADSVKGRFIISRDNAKNSLYLQMNSLRAEDTALYYCAKDGDTSGWYWYGL
DVWGQGTTVTVSS
HCDR1:
(SEQ ID NO: 366)
GFTFDDKA
HCDR2:
(SEQ ID NO: 367)
ISWNSGTI
HCDR3:
(SEQ ID NO: 368)
AKDGDTSGWYWYGLDV
LCVR (V_{L}) Nucleotide Sequence
(SEQ ID NO: 369)
GAAATTGTGTTGACACAGTCTCCTGCCACCCTGTCTTTGTCTCCAGGGGAAAGAG
CCACCCTCTCCTGCAGGGCCAGTCAGAGTGTTAGCAGCTACTTAGCCTGGTACCA
ACAGAAACCTGGCCAGGCTCCCAGGCTCCTCATCCATGATGTATCCAACAGGGCC
ACTGGCATCCCAGCCAGGTTCAGTGGCAGTGGGTCTGGGACAGACTTCACTCTCA
CCATCAGCAGTCTAGAGCCTGAAGATTTTGTAGTTTATTACTGTCAGCAGCGTAG
CGACTGGCCCATCACCTTCGGCCAAGGGACACGACTGGAGATTAAA
LCVR (V_{L}) Amino Acid Sequence
(SEQ ID NO: 370)
EIVLTQSPATLSLSPGERATLSCRASQSVSSYLAWYQQKPGQAPRLLIHDVSNRA
TGIPARFSGSGSGTDFTLTISSLEPEDFVVYYCQQRSDWPITFGQGTRLEIK
LCDR1:
(SEQ ID NO: 371)
QSVSSY
LCDR2:
(SEQ ID NO: 372)
DVS
LCDR3:
(SEQ ID NO: 373)
QQRSDWPIT
69331
HCVR (V_{H}) Nucleotide Sequence
(SEQ ID NO: 374)
CAGGTGCAGCTGGTGGAGTCTGGGGGAGGCGTGGTCCAGCCTGGGAGGTCCCTGA
GACTCTCCTGTATAGCCTCTGGATTCACCTTCAGTGTCTATGGCATTCACTGGGT
CCGCCAGGCTCCAGGCAAGGGGCTGGAGTGGATGGCAGTAATATCACATGATGGA
AATATTAAACACTATGCAGACTCCGTGAAGGGCCGATTCACCATCTCCAGAGACA
ATTCCAAGAACACGCTGTATCTTCAAATTAACAGCCTGAGAACTGAGGACACGGC
TGTGTATTACTGTGCGAAAGATACCTGGAACTCCCTTGATACTTTTGATATCTGG
GGCCAAGGGACAATGGTCACCGTCTCTTCA
HCVR (V_{H}) Amino Acid Sequence
(SEQ ID NO: 375)
QVQLVESGGGVVQPGRSLRLSCIASGFTFSVYGIHWVRQAPGKGLEWMAVISHDG
NIKHYADSVKGRFTISRDNSKNTLYLQINSLRTEDTAVYYCAKDTWNSLDTFDIW
GQGTMVTVSS
HCDR1:
(SEQ ID NO: 376)
GFTFSVYG
HCDR2:
(SEQ ID NO: 377)
ISHDGNIK
HCDR3:
(SEQ ID NO: 378)
AKDTWNSLDTFDI
LCVR (V_{L}) Nucleotide Sequence
(SEQ ID NO: 379)
GACATCCAGTTGACCCAGTCTCCATCCTCCCTGTCTGCATCTGTAGGAGACAGAG
TCACCATCACTTGCTGGGCCAGTCAGGGCATTAGCAGTTATTTAGCCTGGTATCA
GCAAAAACCAGGGAAAGCCCCTAAGCTCCTGATCTATGCTGCATCCACTTTGCAA
AGTGGGGTCCCATCAAGGTTCAGCGGCAGTGGATCTGGGACAGAATTCACTCTCA
CAATCAGCAGCCTGCAGCCTGAAGATTTTGCAACTTATTACTGTCAACAGCTTAA
TAGTTACCCTCTCACTTTCGGCGGAGGGACCAAGGTGGAGATCAAA
LCVR (V_{L}) Amino Acid Sequence
(SEQ ID NO: 380)
DIQLTQSPSSLSASVGDRVTITCWASQGISSYLAWYQQKPGKAPKLLIYAASTLQ
SGVPSRFSGSGSGTEFTLTISSLQPEDFATYYCQQLNSYPLTFGGGTKVEIK
LCDR1:
(SEQ ID NO: 381)
QGISSY
LCDR2:
(SEQ ID NO: 382)
AAS
LCDR3:
(SEQ ID NO: 383)
QQLNSYPLT
69332
HCVR (V_{H}) Nucleotide Sequence
(SEQ ID NO: 384)
CAGGTCACCTTGAGGGAGTCTGGTCCCGCGCTGGTGAAACCCTCACAGACCCTCA
CACTGACCTGCACCTTCTCTGGATTCTCACTCAACACTTATGGGATGTTTGTGAG
CTGGATCCGTCAGCCTCCAGGGAAGGCCCTAGAGTGGCTTGCACACATTCATTGG
GATGATGATAAATACTACAGCACATCTCTGAAGACCAGGCTCACCATCTCCAAGG
ACACCTCCAAAAACCAGGTGGTCCTTACAATGACCAACATGGACCCTGTGGACAC
AGCCACGTATTATTGTGCACGGGGGCACAATAATTTGAACTACATCATCCACTGG
GGCCAGGGAACCCTGGTCACCGTCTCCTCA
HCVR (V_{H}) Amino Acid Sequence
(SEQ ID NO: 385)
QVTLRESGPALVKPSQTLTLTCTFSGFSLNTYGMFVSWIRQPPGKALEWLAHIHW
DDDKYYSTSLKTRLTISKDTSKNQVVLTMTNMDPVDTATYYCARGHNNLNYIIHW
GQGTLVTVSS
HCDR1:
(SEQ ID NO: 386)
GFSLNTYGMF
HCDR2:
(SEQ ID NO: 387)
IHWDDDK
HCDR3:
(SEQ ID NO: 388)
ARGHNNLNYIIH
LCVR (V_{L}) Nucleotide Sequence
(SEQ ID NO: 389)
GCCATCCAGATGACCCAGTCTCCATCCTCCCTGTCTGCATCTGTAGGAGACAGAG
TCACCATCACTTGCCGGGCAAGTCAGGGCATTAGAAATGATTTAGGCTGGTATCA
GCAGAAACCAGGGAAAGCCCCTAAGCTCCTGATCTATGCTGCATCCACTTTACAA
AGTGGGGTCCCATCAAGGTTCAGCGGCAGTGGATCTGGCACAGATTTCACTCTCA
CCATCAGCAGCCTGCAGCCTGAAGATTTTGCAACTTATTACTGTCTACAAGATTA
CAATTACCCATTCACTTTCGGCCCTGGGACCAAAGTGGATATCAAA
LCVR (V_{L}) Amino Acid Sequence
(SEQ ID NO: 390)
AIQMTQSPSSLSASVGDRVTITCRASQGIRNDLGWYQQKPGKAPKLLIYAASTLQ
SGVPSRFSGSGSGTDFTLTISSLQPEDFATYYCLQDYNYPFTFGPGTKVDIK
LCDR1:
(SEQ ID NO: 391)
QGIRND
LCDR2:
(SEQ ID NO: 392)
AAS
LCDR3:
(SEQ ID NO: 393)
LQDYNYPFT
69326
HCVR (V_{H}) Nucleotide Sequence
(SEQ ID NO: 394)
GAGGTGCAGCTGGTGGAGTCTGGGGGAGGCTTGGTACAGCCTGGAGGGTCCCTGA
GACTCTCCTGTGCAGTCTCTGGATTCATCTTCAGTAGTTATGAAATGAACTGGGT
CCGCCAGGCTCCAGGGAAGGGGCTGGAGTGGGTTTCATACATTAGTAGTAGTGGT
AGTACCATATTCTACGCAGACTCTGTGAAGGGCCGATTCACCATCTCCAGAGACA
ACGCCAAGAACTCACTGTATCTGCAAATGAACAGCCTGAGAGCCGAGGACACGGC
TGTTTATTACTGTGTGTCTGGAGTGGTCCTTTTTGATGTCTGGGGCCAAGGGACA
ATGGTCACCGTCTCTTCA
HCVR (V_{H}) Amino Acid Sequence
(SEQ ID NO: 395)
EVQLVESGGGLVQPGGSLRLSCAVSGFIFSSYEMNWVRQAPGKGLEWVSYISSSG
STIFYADSVKGRFTISRDNAKNSLYLQMNSLRAEDTAVYYCVSGVVLFDVWGQGT
MVTVSS
HCDR1:
(SEQ ID NO: 396)
GFIFSSYE
HCDR2:
(SEQ ID NO: 397)
ISSSGSTI
HCDR3:
(SEQ ID NO: 398)
VSGVVLFDV
LCVR (V_{L}) Nucleotide Sequence
(SEQ ID NO: 399)
GAAATAGTGATGACGCAGTCTCCAGCCACCCTGTCTGTGTCTCCGGGGGAAAGAG
CCACCCTCTCCTGCAGGGCCAGTCAGAGTGTTAGCAGCAACTTTGCCTGGTACCA
ACAGAAACCTGGCCAGGCTCCCAGGCTCCTCATCTATAGTGCATCCTCCAGGGCC
ACTGGTATCCCAGTCAGGTTCAGTGGCAGTGGGTCTGGGACAGAGTTCACTCTCA
CCATCAGCAGCCTGCAGTCTGAAGATTTTGCAGTTTATTACTGTCAGCAGTATAA
TATCTGGCCTCGGACGTTCGGCCAAGGGACCAAGGTGGAAATCAAA
LCVR (V_{L}) Amino Acid Sequence
(SEQ ID NO: 400)
EIVMTQSPATLSVSPGERATLSCRASQSVSSNFAWYQQKPGQAPRLLIYSASSRA
TGIPVRFSGSGSGTEFTLTISSLQSEDFAVYYCQQYNIWPRTFGQGTKVEIK
LCDR1:
(SEQ ID NO: 401)
QSVSSN
LCDR2:
(SEQ ID NO: 402)
SAS
LCDR3:
(SEQ ID NO: 403)
QQYNIWPRT
69329
HCVR (V_{H}) Nucleotide Sequence
(SEQ ID NO: 404)
GAGGTGCAGCTGGTGGAGTCTGGGGGAGGCTTGGTCCAGCCTGGGGGGTCCCTGA
GACTCTCCTGTGCAGCCTCTGGATTCACCTTTAGTAACTATTGGATGACCTGGGT
CCGCCAGGCTCCAGGGAAGGGGCTGGAGTGGGTGGCCAACATAAAGGAAGATGGA
AGTGAGAAAGACTATGTGGACTCTGTGAAGGGCCGATTCACCATCTCCAGAGACA
ACGCCAAGAACTCACTGTATCTGCAAATGAACAGCCTGAGAGGCGAGGACACGGC
TGTGTATTACTGTGCGAGAGATGGGGAGCAGCTCGTCGATTACTACTACTACTAC
GTTATGGACGTCTGGGGCCAAGGGACCACGGTCACCGTCTCCTCA
HCVR (V_{H}) Amino Acid Sequence
(SEQ ID NO: 405)
EVQLVESGGGLVQPGGSLRLSCAASGFTFSNYWMTWVRQAPGKGLEWVANIKEDG
SEKDYVDSVKGRFTISRDNAKNSLYLQMNSLRGEDTAVYYCARDGEQLVDYYYYY
VMDVWGQGTTVTVSS
HCDR1:
(SEQ ID NO: 406)
GFTFSNYW
HCDR2:
(SEQ ID NO: 407)
IKEDGSEK
HCDR3:
(SEQ ID NO: 408)
ARDGEQLVDYYYYYVMDV
LCVR (V_{L}) Nucleotide Sequence
(SEQ ID NO: 409)
GACATCCAGATGACCCAGTCTCCATCTTCCGTGTCTGCATCTGTAGGAGACAGAG
TCACCATCACTTGTCGGGCGAGTCAGGGTATTAGCAGCTGGTTAGCCTGGTATCA
GCAGAAACCAGGGAAAGCCCCTAAGCTCCTGATCTATGCTGCATCCAGTTTGCAA
AGTGGGGTCCCATCAAGGTTCAGCGGCAGTGGATCTGGGACAGATTTCACTCTCA
CCATCAGCAGCCTGCAGCCTGAAGATTTTGCAACTTACTATTGTCAAAAGGCTAA
CAGTTTCCCGTACACTTTTGGCCAGGGGACCAAGCTGGAGATCAAA
LCVR (V_{L}) Amino Acid Sequence
(SEQ ID NO: 410)
DIQMTQSPSSVSASVGDRVTITCRASQGISSWLAWYQQKPGKAPKLLIYAASSLQ
SGVPSRFSGSGSGTDFTLTISSLQPEDFATYYCQKANSFPYTFGQGTKLEIK
LCDR1:
(SEQ ID NO: 411)
QGISSW
LCDR2:
(SEQ ID NO: 412)
AAS
LCDR3:
(SEQ ID NO: 413)
QKANSFPYT
69323 (REGN16816 anti-hTfR scFv: hGAA)
HCVR (V_{H}) Nucleotide Sequence
(SEQ ID NO: 414)
GAAGTGCAGCTGGTGGAGTCTGGGGGAGGCTTGGTACAGCCTGGCAGGTCCCTGA
GACTCTCCTGTGCAGCCTCTGGATTCACCTTTGATGACTATGCCATGCACTGGGT
CCGGCAAGCTCCAGGGAAGGGCCTGGAGTGGGTCTCAGGTATTAGTTGGAATAGT
GGTTACATAGGCTATGCGGACTCTGTGAAGGGCCGATTCACCATCTCCAGAGACA
ACGCCGAGAACTCCCTACATCTGCAAATGAACAGTCTGAGAGCTGAGGACACGGC
CTTGTATTACTGTGCAAGAGGGGGATCTACTCTGGTTCGGGGAGTTAAGGGAGGC
TACTACGGTATGGACGTCTGGGGCCAAGGGACCACGGTCACCGTCTCCTCA
HCVR (V_{H}) Amino Acid Sequence
(SEQ ID NO: 415)
EVQLVESGGGLVQPGRSLRLSCAASGFTFDDYAMHWVRQAPGKGLEWVSGISWNS
GYIGYADSVKGRFTISRDNAENSLHLQMNSLRAEDTALYYCARGGSTLVRGVKGG
YYGMDVWGQGTTVTVSS
HCDR1:
(SEQ ID NO: 416)
GFTFDDYA
HCDR2:
(SEQ ID NO: 417)
ISWNSGYI
HCDR3:
(SEQ ID NO: 418)
ARGGSTLVRGVKGGYYGMDV
LCVR (V_{L}) Nucleotide Sequence
(SEQ ID NO: 419)
GACATCCAGATGACCCAGTCTCCATCCTCCCTGTCTGCATCTGTAGGAGACAGAG
TCACCATCACTTGCCGGGCAAGTCAGAGCATAAGTAGCTATTTAAATTGGTATCA
GCAGAAACCAGGTAAAGCCCCTAAGGTCCTGATCTATGCTGCATCCAGTTTGCAA
AGTGGGGTCCCATCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTATTCCGCTCACTTTCGGCGGAGGGACCAAGGTGGAGATCAAA
LCVR (V_{L}) Amino Acid Sequence
(SEQ ID NO: 420)
DIQMTQSPSSLSASVGDRVTITCRASQSISSYLNWYQQKPGKAPKVLIYAASSLQ
SGVPSRFSGSGSGTDFTLTISSLQPEDFATYYCQQSYSIPLTFGGGTKVEIK
LCDR1:
(SEQ ID NO: 421)
QSISSY
LCDR2:
(SEQ ID NO: 422)
AAS
LCDR3:
(SEQ ID NO: 423)
QQSYSIPLT
69305
HCVR (V_{H}) Nucleotide Sequence
(SEQ ID NO: 424)
CAGGTGCAGCTGGTGGAGTCTGGGGGAGGCGTGGTCCAGCCTGGGAGGTCCCTGA
GACTCTCCTGTGCAGCGTCTGGATTCACCTTCAGTAGCTATGGCATGCACTGGGT
CCGCCAGGCTCCAGGCAAGGGGCTGGAGTGGGTGGCAGTTATATGGTATGATGGA
AGTAATAAATACTATGCAGACTCCGTGAAGGGCCGATTCACCATCTCCAGAGACA
TTTCCAAGAACACGCTGTATCTGCAAATGAACAGCCTGAGAGCCGAGGACACGGC
CGTATATTACTGTGCGGGTCAACTGGATCTCTTCTTTGACTACTGGGGCCAGGGA
ACCCTGGTCACCGTCTCCTCA
HCVR (V_{H}) Amino Acid Sequence
(SEQ ID NO: 425)
QVQLVESGGGVVQPGRSLRLSCAASGFTFSSYGMHWVRQAPGKGLEWVAVIWYDG
SNKYYADSVKGRFTISRDISKNTLYLQMNSLRAEDTAVYYCAGQLDLFFDYWGQG
TLVTVSS
HCDR1:
(SEQ ID NO: 426)
GFTFSSYG
HCDR2:
(SEQ ID NO: 427)
IWYDGSNK
HCDR3:
(SEQ ID NO: 428)
AGQLDLFFDY
LCVR (V_{L}) Nucleotide Sequence
(SEQ ID NO: 429)
GACATCCAGATGACCCAGTCTCCATCCTCCCTGTCTGCATCTGTAGGAGACAGAG
TCACCATCACTTGCCGGGCAAGTCAGAGCATTGACAGGTATTTAAATTGGTATCG
GCAGAAACCAGGGAAAGCCCCTAAGCTCCTGATCTATACTACATCCAGTTTGCAA
AGTGGGGTCCCATCAAGGTTCAGTGGCAGTGGATCTGGGACAGATTTCACTCTCA
CCCTCAGCAGTCTGCAGCCTGAAGATTTTGCAACTTACTACTGTCAGCAGAGTTA
CAGTCCCCCGCTCACTTTCGGCGGAGGGACCAAGGTGGAGATCAAA
LCVR (V_{L}) Amino Acid Sequence
(SEQ ID NO: 430)
DIQMTQSPSSLSASVGDRVTITCRASQSIDRYLNWYRQKPGKAPKLLIYTTSSLQ
SGVPSRFSGSGSGTDFTLTLSSLQPEDFATYYCQQSYSPPLTFGGGTKVEIK
LCDR1:
(SEQ ID NO: 431)
QSIDRY
LCDR2:
(SEQ ID NO: 432)
TTS
LCDR3:
(SEQ ID NO: 433)
QQSYSPPLT
69307 (REGN16817 anti-hTfR scFv: hGAA)
HCVR (V_{H}) Nucleotide Sequence
(SEQ ID NO: 434)
GAGGTGCAGCTGGTGGAGTCTGGGGGAGGCTTGGTCCAGCCTGGGGGGTCCCTGA
GACTCTCCTGTACAGCCTCTGGATTCACCTTTAGTAACTATTGGATGACCTGGGT
CCGCCAGGCTCCAGGGAAGGGGCTGGAGTGGGTGGCCAACATAAAGGAAGATGGA
AGTGAGAAAGAGTATGTGGACTCTGTGAAGGGCCGGTTCACCATCTCCAGAGACA
ACGCCAAGAATTCACTGTATCTGCAAATGAACAGCCTGAGAGGCGAGGACACGGC
TGTATATTACTGTGCGAGAGATGGGGAGCAGCTCGTCGATTACTATTACTACTAC
GTTATGGACGTCTGGGGCCAAGGGACCACGGTCACCGTCTCCTCA
HCVR (V_{H}) Amino Acid Sequence
(SEQ ID NO: 435)
EVQLVESGGGLVQPGGSLRLSCTASGFTFSNYWMTWVRQAPGKGLEWVANIKEDG
SEKEYVDSVKGRFTISRDNAKNSLYLQMNSLRGEDTAVYYCARDGEQLVDYYYYY
VMDVWGQGTTVTVSS
HCDR1:
(SEQ ID NO: 436)
GFTFSNYW
HCDR2:
(SEQ ID NO: 437)
IKEDGSEK
HCDR3:
(SEQ ID NO: 438)
ARDGEQLVDYYYYYVMDV
LCVR (V_{L}) Nucleotide Sequence
(SEQ ID NO: 439)
GACATCCAGATGACCCAGTCTCCATCTTCCGTGTCTGCATCTGTTGGAGACAGAG
TCACCATCACTTGTCGGGCGAGTCAGGGTATTAGCAGCTGGTTAGCCTGGTATCA
GCAGAAACCAGGGAAAGCCCCTAAGCTCCTGATCTATGCTGCATCCAGTTTGCAA
AGTGGGGTCCCATCAAGGTTCAGCGGCAGTGGATCTGGGACAGATTTCACTCTCA
CCATCAGCAGCCTGCAGCCTGAAGATTTTGCAACTTACTATTGTCAAAAGGCTGA
CAGTCTCCCGTACGCTTTTGGCCAGGGGACCAAGCTGGAGATCAAA
LCVR (V_{L}) Amino Acid Sequence
(SEQ ID NO: 440)
DIQMTQSPSSVSASVGDRVTITCRASQGISSWLAWYQQKPGKAPKLLIYAASSLQ
SGVPSRFSGSGSGTDFTLTISSLQPEDFATYYCQKADSLPYAFGQGTKLEIK
LCDR1:
(SEQ ID NO: 441)
QGISSW
LCDR2:
(SEQ ID NO: 442)
AAS
LCDR3:
(SEQ ID NO: 443)
QKADSLPYA
12795B
HCVR (V_{H}) Nucleotide Sequence
(SEQ ID NO: 444)
GAGGTGCAGCTGGTGGAGTCTGGGGGAGGCTTGGTTCAGCCTGGGGGGTCCCTGA
GACTCTCCTGTGCAACCTCTGGATTCACCTTTACCAGCTATGACATGAAGTGGGT
CCGCCAGGCTCCAGGGCTGGGCCTGGAGTGGGTCTCAGCTATTAGTGGTAGTGGT
GGTAACACATACTACGCAGACTCCGTGAAGGGCCGGTTCACCATCTCCAGAGACA
ATTCCAGGAACACGCTGTATCTGCAAATGAACAGCCTGAGAGCCGAGGACACGGC
CGTATATTACTGTACGAGGTCCCATGACTTCGGTGCCTTCGACTACTTTGACTAC
TGGGGCCAGGGAACCCTGGTCACCGTCTCCTCA
HCVR (V_{H}) Amino Acid Sequence
(SEQ ID NO: 445)
EVQLVESGGGLVQPGGSLRLSCATSGFTFTSYDMKWVRQAPGLGLEWVSAISGSG
GNTYYADSVKGRFTISRDNSRNTLYLQMNSLRAEDTAVYYCTRSHDFGAFDYFDY
WGQGTLVTVSS
HCDR1:
(SEQ ID NO: 446)
GFTFTSYD
HCDR2:
(SEQ ID NO: 447)
ISGSGGNT
HCDR3:
(SEQ ID NO: 448)
TRSHDFGAFDYFDY
LCVR (V_{L}) Nucleotide Sequence
(SEQ ID NO: 449)
GACATCCAGATGACCCAGTCTCCATCCTCCCTGTCTGCATCTGTGGGAGACAGAG
TCACCATCACTTGCCGGGCAAGTCAGGGCATTAGAGATCATTTTGGCTGGTATCA
GCAGAAACCAGGGAAAGCCCCTAAGCGCCTGATCTATGCTGCATCCAGTTTGCAC
AGTGGGGTCCCATCAAGGTTCAGCGGCAGTGGATCTGGGACAGAATTCACTCTCA
CAATCAGCAGCTTGCAGCCTGAAGATTTTGCAACCTATTACTGTCTACAGTATGA
TACTTACCCGCTCACTTTCGGCGGAGGGACCAAGGTGGAGATCAAA
LCVR (V_{L}) Amino Acid Sequence
(SEQ ID NO: 450)
DIQMTQSPSSLSASVGDRVTITCRASQGIRDHFGWYQQKPGKAPKRLIYAASSLH
SGVPSRFSGSGSGTEFTLTISSLQPEDFATYYCLQYDTYPLTFGGGTKVEIK
LCDR1:
(SEQ ID NO: 451)
QGIRDH
LCDR2:
(SEQ ID NO: 452)
AAS
LCDR3:
(SEQ ID NO: 453)
LQYDTYPLT
12798B (REGN17078 Fab; REGN17072 scFv;
REGN16818 anti-hTfR scFv: hGAA)
HCVR (V_{H}) Nucleotide Sequence
(SEQ ID NO: 454)
GAAGTGCAGCTGGTGGAGTCTGGGGGAGACTTGGTACAGCCTGGCAGGTCCCTGA
GACTCTCCTGTGCAGCCTCTGGATTCACCTTTGATGATTATGCCATGCACTGGGT
CCGGCAAGCTCCAGGGAAGGGCCTGGAGTGGGTCTCAGGTATTAGTTGGAATAGT
GCTACCAGAGTCTATGCGGACTCTGTGAAGGGCCGATTCACCATCTCCAGAGACA
ACGCCAAGAATTTCCTGTATCTGCAAATGAACAGTCTGAGATCTGAGGACACGGC
CTTGTATCACTGTGCAAAAGATATGGATATCTCGCTAGGGTACTACGGTTTGGAC
GTCTGGGGCCAAGGGACCACGGTCACCGTCTCCTCA
HCVR (V_{H}) Amino Acid Sequence
(SEQ ID NO: 455)
EVQLVESGGDLVQPGRSLRLSCAASGFTFDDYAMHWVRQAPGKGLEWVSGISWNS
ATRVYADSVKGRFTISRDNAKNFLYLQMNSLRSEDTALYHCAKDMDISLGYYGLD
VWGQGTTVTVSS
HCDR1:
(SEQ ID NO: 456)
GFTFDDYA
HCDR2:
(SEQ ID NO: 457)
ISWNSATR
HCDR3:
(SEQ ID NO: 458)
AKDMDISLGYYGLDV
LCVR (V_{L}) Nucleotide Sequence
(SEQ ID NO: 459)
GAAATAGTGATGACGCAGTCTCCAGCCACCCTGTCTGTGTCTCCAGGGGAAAGAG
CCACCCTCTCCTGCAGGGCCAGTCAGACTGTTAGCAGCAACTTAGCCTGGTATCA
GCAGAAACCTGGCCAGGCTCCCAGGCTCCTCATCTATGGTTCATCCTCCAGGGCC
ACTGGTATCCCAGCCAGGTTCAGTGGCAGTGGGTCTGGGACAGAGTTCACTCTCA
CCATCAGCAGCCTGCAGTCTGAAGATTTTGCAGTTTATTACTGTCAGCAGTATAA
TAACTGGCCTCCCTACACTTTTGGCCAGGGGACCAAGCTGGAGATCAAA
LCVR (V_{L}) Amino Acid Sequence
(SEQ ID NO: 460)
EIVMTQSPATLSVSPGERATLSCRASQTVSSNLAWYQQKPGQAPRLLIYGSSSRA
TGIPARFSGSGSGTEFTLTISSLQSEDFAVYYCQQYNNWPPYTFGQGTKLEIK
LCDR1:
(SEQ ID NO: 461)
QTVSSN
LCDR2:
(SEQ ID NO: 462)
GSS
LCDR3:
(SEQ ID NO: 463)
QQYNNWPPYT
12799B (REGN17079 Fab; REGN17073 scFv;
REGN16819 anti-hTfR scFv: hGAA)
HCVR (V_{H}) Nucleotide Sequence
(SEQ ID NO: 464)
CAGATCACCTTGAAGGAGTCTGGTCCTACGCTGGTGAAACCCACACAGACCCTCA
CGCTGACCTGCACCTTCTCTGGGTTCTCACTCAGCACTAGTGGAGTGGGTGTGGT
CTGGATCCGTCAGCCCCCCGGAAAGGCCCTGGAGTGGCTTGCACTCATTTATTGG
AATGATCATAAGCGGTACAGCCCATCTCTGGGGAGCAGGCTCACCATCACCAAGG
ACACCTCCAAAAACCAGGTGGTCCTTACAATGACCAACATGGACCCTGTGGACAC
AGCCACATATTACTGTGCACACTACAGTGGGAGCTATTCCTACTACTACTATGGT
TTGGACGTCTGGGGCCAAGGGACCACGGTCACCGTCTCCTCA
HCVR (V_{H}) Amino Acid Sequence
(SEQ ID NO: 465)
QITLKESGPTLVKPTQTLTLTCTFSGFSLSTSGVGVVWIRQPPGKALEWLALIYW
NDHKRYSPSLGSRLTITKDTSKNQVVLTMTNMDPVDTATYYCAHYSGSYSYYYYG
LDVWGQGTTVTVSS
HCDR1:
(SEQ ID NO: 466)
GFSLSTSGVG
HCDR2:
(SEQ ID NO: 467)
IYWNDHK
HCDR3:
(SEQ ID NO: 468)
AHYSGSYSYYYYGLDV
LCVR (V_{L}) Nucleotide Sequence
(SEQ ID NO: 469)
GACATCCAGATGACCCAGTCTCCATCTTCCGTGTCTGCATCTGTAGGAGACAGAG
TCACCATCACTTGTCGGGCGAGTCAGGGTATTGCCAGCTGGTTAGCCTGGTATCA
GCAGAAACCAGGGAAAGCCCCTGAGCTCCTGATCTATGCTGCATCCAGTTTGCAA
GGTGGGGTCCCATCAAGGTTCAGCGGCAGTGGATCTGGGACAGATTTCACTCTCA
CCATCAGCAGCCTGCAGCCTGAAGATTTTGCAATTTACTATTGTCAACAGGCTAA
CTATTTCCCGTGGACGTTCGGCCAAGGGACCAAGGTGGAAATCAAA
LCVR (V_{L}) Amino Acid Sequence
(SEQ ID NO: 470)
DIQMTQSPSSVSASVGDRVTITCRASQGIASWLAWYQQKPGKAPELLIYAASSLQ
GGVPSRFSGSGSGTDFTLTISSLQPEDFAIYYCQQANYFPWTFGQGTKVEIK
LCDR1:
(SEQ ID NO: 471)
QGIASW
LCDR2:
(SEQ ID NO: 472)
AAS
LCDR3:
(SEQ ID NO: 473)
QQANYFPWT
12801B
HCVR (V_{H}) Nucleotide Sequence
(SEQ ID NO: 474)
GAGGTGCAGCTGTTGGAGTCTGGGGGAGCCTTGGTACAGCCTGGGGGGTCCCTGA
GACTCTCCTGTGCAGCCTCTGGATTCACCTTTACCTCCTATGCCATGCACTGGGT
CCGCCAGGCTCCAGGGAAGGGTCTGGAGTGGGTCTCATCTATTAGAGGTAGTGGT
GGTGGCACATACTCCGCAGACTCCGTGAAGGGCCGGTTCACCATCTCCAGAGACA
ATTCCAGGGACACTCTATATCTGCAAATGAACAGTGTGAGAGCCGAGGACACGGC
CGTTTATTACTGTGCGAGGTCCCATGACTACGGTGCCTTCGACTTCTTTGACTAC
TGGGGCCAGGGAACCCTGGTCACCGTCTCCTCA
HCVR (V_{H}) Amino Acid Sequence
(SEQ ID NO: 475)
EVQLLESGGALVQPGGSLRLSCAASGFTFTSYAMHWVRQAPGKGLEWVSSIRGSG
GGTYSADSVKGRFTISRDNSRDTLYLQMNSVRAEDTAVYYCARSHDYGAFDFFDY
WGQGTLVTVSS
HCDR1:
(SEQ ID NO: 476)
GFTFTSYA
HCDR2:
(SEQ ID NO: 477)
IRGSGGGT
HCDR3:
(SEQ ID NO: 478)
ARSHDYGAFDFFDY
LCVR (V_{L}) Nucleotide Sequence
(SEQ ID NO: 479)
GACATCCAGATGACCCAGTCTCCATCCTCCCTGTCTGCATCTGTAGGAGACAGAG
TCACCATCACTTGCCGGGCAAGTCAGGGCATTAGAACTGATTTAGGCTGGTATCA
GCAGAAACCAGGGAAAGCCCCTAAGCGCCTGATCTATGCTGCATCCAGTTTGCAA
AGTGGGGTCCCATCAAGGTTCAGCGGCAGTGGATCTGGGACAGAATTCACTCTCA
CAATCAGCAGCCTGCGGCCTGAAGATTTTGCAACTTTTTACTGTCTACAGTATAA
TAGTTACCCGCTCACTTTCGGCGGAGGGACCAAGGTGGAGATCAAA
LCVR (V_{L}) Amino Acid Sequence
(SEQ ID NO: 480)
DIQMTQSPSSLSASVGDRVTITCRASQGIRTDLGWYQQKPGKAPKRLIYAASSLQ
SGVPSRFSGSGSGTEFTLTISSLRPEDFATFYCLQYNSYPLTFGGGTKVEIK
LCDR1:
(SEQ ID NO: 481)
QGIRTD
LCDR2:
(SEQ ID NO: 482)
AAS
LCDR3:
(SEQ ID NO: 483)
LQYNSYPLT
12802B (REGN16820 anti-hTfR scFv: hGAA)
HCVR (V_{H}) Nucleotide Sequence
(SEQ ID NO: 484)
CAGGTGCAGCTGGTGGAGTCTGGGGGAGGCTTGGTCAAGCCTGGAGGGTCCCTGA
GACTCTCCTGTGCAGCCTCTGGATTCACCTTCAGTGACTACTTCATGAGCTGGAT
CCGCCAGGCTCCAGGGAAGGGGCTGGAGTGGGTTTCATACATTAGTAGTACTGGT
AGTACCATAAATTATGCAGACTCTGTGAAGGGCCGATTCACCATCTCCAGGGACA
ATGTCAAGAATTCACTGTATCTGCAAATGACCAGCCTGAGAGTCGAGGACACGGC
CGTGTATTACTGTACGAGAGATAACTGGAACTATGAATACTGGGGCCAGGGAACC
CTGGTCACCGTCTCCTCA
HCVR (V_{H}) Amino Acid Sequence
(SEQ ID NO: 485)
QVQLVESGGGLVKPGGSLRLSCAASGFTFSDYFMSWIRQAPGKGLEWVSYISSTG
STINYADSVKGRFTISRDNVKNSLYLQMTSLRVEDTAVYYCTRDNWNYEYWGQGT
LVTVSS
HCDR1:
(SEQ ID NO: 486)
GFTFSDYF
HCDR2:
(SEQ ID NO: 487)
ISSTGSTI
HCDR3:
(SEQ ID NO: 488)
TRDNWNYEY
LCVR (V_{L}) Nucleotide Sequence
(SEQ ID NO: 489)
GAAATAGTGATGACGCAGTCTCCAGCCACCCTGTCTGTGTCTCCAGGGGAAAGAG
CCACCCTCTCCTGCAGGGCCAGTCAGAGTGTTAGCATCAACTTAGCCTGGTACCA
GCAGAAACCTGGCCAGGCTCCCAGGCTCCTCATCTTTGTTGCATCCACCAGGGCC
ACTGGTATCCCAGCCAGGTTCAGTGGCAGTGGGTCTGGGACAGAGTTCACTCTCA
CCATCAGCAGCCTGCAGTCTGAAGATTTTGCAACTTATTACTGTCAGCAGTATGA
TATCTGGCCGTACACTTTTGGCCAGGGGACCAAGCTGGAGATCAAA
LCVR (V_{L}) Amino Acid Sequence
(SEQ ID NO: 490)
EIVMTQSPATLSVSPGERATLSCRASQSVSINLAWYQQKPGQAPRLLIFVASTRA
TGIPARFSGSGSGTEFTLTISSLQSEDFATYYCQQYDIWPYTFGQGTKLEIK
LCDR1:
(SEQ ID NO: 491)
QSVSIN
LCDR2:
(SEQ ID NO: 492)
VAS
LCDR3:
(SEQ ID NO: 493)
QQYDIWPYT
12808B
HCVR (V_{H}) Nucleotide Sequence
(SEQ ID NO: 494)
CAGCTGCAGCTGCAGGAGTCGGGCCCAGGACTGGTGAAGCCTTCGGAGACCCTGT
CCCTCACCTGCACTGTGTCTGGTGAATCCATCAGCAGTAATACTTACTACTGGGG
CTGGATCCGCCAGCCCCCAGGGAAGGGGCTGGAATGGATTGGGAGTATCGATTAT
AGTGGGACCACCAATTATAACCCGTCCCTCAAGAGTCGAGTCACCATATCCGTAG
ACACGTCCAGGAATCACTTCTCCCTGAGGCTGAGGTCTGTGACCGCCGCAGACAC
GGCTGTGTATTACTGTGCGAGAGAGTGGGGAAACTACGGCTACTATTACGGTATG
GACGTTTGGGGCCAAGGGACCACGGTCACCGTCTCCTCA
HCVR (V_{H}) Amino Acid Sequence
(SEQ ID NO: 495)
QLQLQESGPGLVKPSETLSLTCTVSGESISSNTYYWGWIRQPPGKGLEWIGSIDY
SGTTNYNPSLKSRVTISVDTSRNHFSLRLRSVTAADTAVYYCAREWGNYGYYYGM
DVWGQGTTVTVSS
HCDR1:
(SEQ ID NO: 496)
GESISSNTYY
HCDR2:
(SEQ ID NO: 497)
IDYSGTT
HCDR3:
(SEQ ID NO: 498)
AREWGNYGYYYGMDV
LCVR (V_{L}) Nucleotide Sequence
(SEQ ID NO: 499)
GACATCCAGATGACCCAGTCTCCATCCTCCCTGTCTGCATCTGTAGGAGACAGAG
TCACCATCAATTGCCGGGCAAGTCAGGGCATTAGAAATGATTTAGGCTGGTATCA
GCAGAAACCAGGGAAAGCCCCTAAGCGCCTGATCTATGCTGCATCCAGTTTGCAA
AGTGGGGTCCCATTAAGGTTCAGTGGCAGTGGATCTGGGACAGAATTCACTCTCA
CAATCAACAACCTGCAGCCTGAAGATTTTGCAACTTATTACTGTCTATCGCATAA
TAGTTACCCGTGGACGTTCGGCCAAGGGACCAAGGTGGAAATCAAA
LCVR (V_{L}) Amino Acid Sequence
(SEQ ID NO: 500)
DIQMTQSPSSLSASVGDRVTINCRASQGIRNDLGWYQQKPGKAPKRLIYAASSLQ
SGVPLRFSGSGSGTEFTLTINNLQPEDFATYYCLSHNSYPWTFGQGTKVEIK
LCDR1:
(SEQ ID NO: 501)
QGIRND
LCDR2:
(SEQ ID NO: 502)
AAS
LCDR3:
(SEQ ID NO: 503)
LSHNSYPWT
12812B (REGN16821 anti-hTfR scFv: hGAA)
HCVR (V_{H}) Nucleotide Sequence
(SEQ ID NO: 504)
CAGGTGCAGCTGGTGCAGTCTGGGGCTGAGGTGAAGAAGCCTGGGTCCTCGGTGA
GGGTCTCCTGCAAGGCTTCTAGAGGCACCTTCAGCAGCTATGCTATCAGCTGGGT
GCGACAGGCCCCTGGACAAGGCCTTGAGTGGATGGGAGGGATCATCCCCATCTTT
GGTACAGCAAACTACGCACAGAAGTTCCTGGCCAGAGTCACGATTACCGCGGACG
AATCCACGAGCACAGCCTACATGGAGCTGAGCAGCCTGAGATCTGAGGACACGGC
CGTGTATTACTGTGCGAGAGAGAAGGGGTGGAACTACTTTGACTACTGGGGCCAG
GGAACCCTGGTCACCGTCTCCTCA
HCVR (V_{H}) Amino Acid Sequence
(SEQ ID NO: 505)
QVQLVQSGAEVKKPGSSVRVSCKASRGTFSSYAISWVRQAPGQGLEWMGGIIPIF
GTANYAQKFLARVTITADESTSTAYMELSSLRSEDTAVYYCAREKGWNYFDYWGQ
GTLVTVSS
HCDR1:
(SEQ ID NO: 506)
RGTFSSYA
HCDR2:
(SEQ ID NO: 507)
IIPIFGTA
HCDR3:
(SEQ ID NO: 508)
AREKGWNYFDY
LCVR (V_{L}) Nucleotide Sequence
(SEQ ID NO: 509)
GACATCCAGATGACCCAGTCTCCACCTTCCGTGTCTGCATCTGTAGGAGACAGAG
TCACCATCACTTGTCGGGCGAGTCAGGGTATTAGCAGCTGGTTAGCCTGGTATCA
GCAGAAACCAGGGAAAGCCCCTAAACTCCTGATCTATGCTGCATCCAGTTTGCAA
AGTGGGGTCCCATCAAGGTTCAGCGGCAGTGGATCTGGGACAGATTTCACTCTCA
CCATCAGCAGCCTGCAGCCTGAAGATTTTGCAACTTACTATTGTCAACAGGCTAA
CAGTTTCCCTCGGACGTTCGGCCAAGGGACCAAGGTGGAAATCAAA
LCVR (V_{L}) Amino Acid Sequence
(SEQ ID NO: 510)
DIQMTQSPPSVSASVGDRVTITCRASQGISSWLAWYQQKPGKAPKLLIYAASSLQ
SGVPSRFSGSGSGTDFTLTISSLQPEDFATYYCQQANSFPRTFGQGTKVEIK
LCDR1:
(SEQ ID NO: 511)
QGISSW
LCDR2:
(SEQ ID NO: 512)
AAS
LCDR3:
(SEQ ID NO: 513)
QQANSFPRT
12816B
HCVR (V_{H}) Nucleotide Sequence
(SEQ ID NO: 514)
CAGGTGCAGCTGGTGGAGTCTGGGGGAGGCTTGGTCAAGCCTGGAGGGTCCCTGA
GACTCTCCTGTGCAGCCTCTGGATTCACCTTCAGTGACTACTACATGAACTGGAT
CCGCCAGGCTCCAGGGAAGGGGCTGGAGTGGGTTTCATACATTAGTAGTAGTGGG
ACTACCATATACTACGCAGACTCTGTGAAGGGCCGATTCACCATCTCCAGGGACA
ACGCCAAGAAATCACTGTATCTGGAGATGAACAGCCTCAGAGCCGAGGACACGGC
CGTGTACTACTGTGCGAGAGAGGGGTACGGTAATGACTACTATTACTACGGTATA
GACGTCTGGGGCCAAGGGACCACGGTCACCGTCTCCTCA
HCVR (V_{H}) Amino Acid Sequence
(SEQ ID NO: 515)
QVQLVESGGGLVKPGGSLRLSCAASGFTFSDYYMNWIRQAPGKGLEWVSYISSSG
TTIYYADSVKGRFTISRDNAKKSLYLEMNSLRAEDTAVYYCAREGYGNDYYYYGI
DVWGQGTTVTVSS
HCDR1:
(SEQ ID NO: 516)
GFTFSDYY
HCDR2:
(SEQ ID NO: 517)
ISSSGTTI
HCDR3:
(SEQ ID NO: 518)
AREGYGNDYYYYGIDV
LCVR (V_{L}) Nucleotide Sequence
(SEQ ID NO: 519)
GATATTGTGATGACTCAGTCTCCACTCTCCCTGCCCGTCACCCCTGGAGAGCCGG
CCTCCATCTCCTGCAGGTCTAGTCAGAGCCTCCTGCATGGTAATGGATACAACTA
TTTGACTTGGTACCTGCAGAAGCCAGGGCAGTCTCCACAGCTCCTGATCTATTTG
GGTTCTAATCGGGCCTCCGGGGTCCCTGACAGGTTCAGTGGCAGTGGATCAGGCA
CAGATTTTACACTGAAAATAAGCAGAGTGGAGGCTGAGGATGTTGGGGTTTATTA
CTGCATGCAAGCTCTACAAACTCCGTACACTTTTGGCCAGGGGACCAAGCTGGAG
ATCAAA
LCVR (V_{L}) Amino Acid Sequence
(SEQ ID NO: 520)
DIVMTQSPLSLPVTPGEPASISCRSSQSLLHGNGYNYLTWYLQKPGQSPQLLIYL
GSNRASGVPDRFSGSGSGTDFTLKISRVEAEDVGVYYCMQALQTPYTFGQGTKLE
IK
LCDR1:
(SEQ ID NO: 521)
QSLLHGNGYNY
LCDR2:
(SEQ ID NO: 522)
LGS
LCDR3:
(SEQ ID NO: 523)
MQALQTPYT
12833B
HCVR (V_{H}) Nucleotide Sequence
(SEQ ID NO: 524)
CAGGTGCAGCTGGTGGAGTCTGGGGGAGGCGTGGTCCAGCCTGGGAGGTCCCTGA
GACTCTCCTGTGCAGCCTCTGGATTCACCTTCAGTAGCTTTGGCATGCACTGGGT
CCGCCAGGCTCCAGGCAAGGGGCTGGAGTGGGTGATATTTATATCATATGATGGA
AGTGATAAATACTATGCAGACTCCGTGAAGGGCCGATTCGCCATCTCCAGAGACA
GTTCCAAGAACACGCTATATCTGCAAATGAACAGCCTGAGAGCTGAGGACACGGC
TGTGTATTACTGTGCGAAAGAAAACGGTATTTTGACTGATTCCTACGGTATGGAC
GTCTGGGGCCAAGGGACCACGGTCACCGTCTCCTCA
HCVR (V_{H}) Amino Acid Sequence
(SEQ ID NO: 525)
QVQLVESGGGVVQPGRSLRLSCAASGFTFSSFGMHWVRQAPGKGLEWVIFISYDG
SDKYYADSVKGRFAISRDSSKNTLYLQMNSLRAEDTAVYYCAKENGILTDSYGMD
VWGQGTTVTVSS
HCDR1:
(SEQ ID NO: 526)
GFTFSSFG
HCDR2:
(SEQ ID NO: 527)
ISYDGSDK
HCDR3:
(SEQ ID NO: 528)
AKENGILTDSYGMDV
LCVR (V_{L}) Nucleotide Sequence
(SEQ ID NO: 529)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 530)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 531)
QSISSY
LCDR2:
(SEQ ID NO: 532)
AAS
LCDR3:
(SEQ ID NO: 533)
QQSYSTPPIT
12834B
HCVR (V_{H}) Nucleotide Sequence
(SEQ ID NO: 534)
CAGGTTCAGCTGGTGCAGTCTGGAGCTGAGGTGAAGAAGCCTGGGGCCTCTGTGA
AGGTCTCCTGCAAGGCTTCTGGTTACACCTTTACCAGCTATGGTATCAGCTGGGT
GCGACAGGCCCCTGGACAAGGGCTTGAGTGGATGGGATGGATCAGTGTTTACCAT
GGTAACACAAACTATGCACAGAAGTTCCAGGGCAGAGTCACCATGACCACAGACA
CATCCACGAGCACAGCCTACATGGAGCTGAGGAGCCTGAGATCTGACGACACGGC
CGTGTATTACTGTGCGAGAGAGGGGTATTACGATTTTTGGAGTGGTTATTACCCT
TTTGACTACTGGGGCCAGGGAACCCTGGTCACCGTCTCCTCA
HCVR (V_{H}) Amino Acid Sequence
(SEQ ID NO: 535)
QVQLVQSGAEVKKPGASVKVSCKASGYTFTSYGISWVRQAPGQGLEWMGWISVYH
GNTNYAQKFQGRVTMTTDTSTSTAYMELRSLRSDDTAVYYCAREGYYDFWSGYYP
FDYWGQGTLVTVSS
HCDR1:
(SEQ ID NO: 536)
GYTFTSYG
HCDR2:
(SEQ ID NO: 537)
ISVYHGNT
HCDR3:
(SEQ ID NO: 538)
AREGYYDFWSGYYPFDY
LCVR (V_{L}) Nucleotide Sequence
(SEQ ID NO: 539)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 540)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 541)
QSISSY
LCDR2:
(SEQ ID NO: 542)
AAS
LCDR3:
(SEQ ID NO: 543)
QQSYSTPPIT
12835B
HCVR (V_{H}) Nucleotide Sequence
(SEQ ID NO: 544)
GAGGTGCAGCTGGTGGAGTCTGGGGGAGGCTTGATACAACCTGGAGGGTCCCTGA
GACTCTCCTGTGAAGCCTCTGGATTCACCTTCAGAAATTATGAAATGAATTGGGT
CCGCCAGGCTCCAGGGAAGGGGCTGGAGTGGGTTTCATATATTAGTAGTAGTGGT
AATATGAAAGACTACGCAGAGTCTGTGAAGGGCCGATTCACCATCTCCAGAGACA
ATGTCAAGAATTCACTGCAGCTGCAAATGAACAGCCTGAGAGTCGAGGACACGGC
TGTTTATTACTGTGCGAGAGACGAGTTTCCTTACGGAATGGACGTCTGGGGCCAA
GGGACCACGGTCACCGTCTCCTCA
HCVR (V_{H}) Amino Acid Sequence
(SEQ ID NO: 545)
EVQLVESGGGLIQPGGSLRLSCEASGFTFRNYEMNWVRQAPGKGLEWVSYISSSG
NMKDYAESVKGRFTISRDNVKNSLQLQMNSLRVEDTAVYYCARDEFPYGMDVWGQ
GTTVTVSS
HCDR1:
(SEQ ID NO: 546)
GFTFRNYE
HCDR2:
(SEQ ID NO: 547)
ISSSGNMK
HCDR3:
(SEQ ID NO: 548)
ARDEFPYGMDV
LCVR (V_{L}) Nucleotide Sequence
(SEQ ID NO: 549)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 550)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 551)
QSISSY
LCDR2:
(SEQ ID NO: 552)
AAS
LCDR3:
(SEQ ID NO: 553)
QQSYSTPPIT
12847B (REGN17083 anti-hTfR Fab; REGN17077 anti-hTfR scFv;
REGN16826 anti-hTfR scFv: hGAA)
HCVR (V_{H}) Nucleotide Sequence
(SEQ ID NO: 554)
GAAGTGCAGCTGGTGGAGTCTGGGGGAGGCTTGGTTCAGCCTGGCAGGTCCCTGA
GACTCTCCTGTGCAGCCTCTGGATTCACCTTTGATGATTATGCCATGAACTGGGT
CCGGCAAGCTCCAGGGAAGGGCCTGGAGTGGGTCTCAGGTATTAGTTGGAGTAGT
GGTAGCATGGACTATGCGGACTCTGTGAAGGGCCGATTCACCATCTCCAGAGACA
ACGCCAAAAACTCCCTGTATCTGCAAATGAACAGTCTGAGAACTGAGGACACGGC
CTTATATTACTGTGCAAAAGCTAGGGAAGTTGGAGACTACTACGGTATGGACGTC
TGGGGCCAAGGGACCACGGTCACCGTCTCCTCA
HCVR (V_{H}) Amino Acid Sequence
(SEQ ID NO: 555)
EVQLVESGGGLVQPGRSLRLSCAASGFTFDDYAMNWVRQAPGKGLEWVSGISWSS
GSMDYADSVKGRFTISRDNAKNSLYLQMNSLRTEDTALYYCAKAREVGDYYGMDV
WGQGTTVTVSS
HCDR1:
(SEQ ID NO: 556)
GFTFDDYA
HCDR2:
ISWSSGSM
(SEQ ID NO: 557)
HCDR3:
(SEQ ID NO: 558)
AKAREVGDYYGMDV
LCVR (V_{L}) Nucleotide Sequence
(SEQ ID NO: 559)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 560)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 561)
QSISSY
LCDR2:
(SEQ ID NO: 562)
AAS
LCDR3:
(SEQ ID NO: 563)
QQSYSTPPIT
12848B (REGN16827 anti-hTfR scFv: hGAA)
HCVR (V_{H}) Nucleotide Sequence
(SEQ ID NO: 564)
GAAGTGCAGCTGGTGGAGTCTGGGGGAGGCTTGGTACAGCCTGGCAGGTCCCTGA
CACTCTCCTGTGCAGCCTCTGGATTCACCTTTGATAATTTTGGCATGCACTGGGT
CCGGCAAGGTCCAGGGAAGGGCCTGGAATGGGTCTCAGGTCTTACTTGGAATAGT
GGTGTCATAGGCTATGCGGACTCTGTGAAGGGCCGATTCACCATCTCCAGAGACA
ACGCCAAGAACTCCCTGTATCTGCAAATGAACAGTCTGAGACCTGAGGACACGGC
CTTATATTACTGTGCAAAAGATATACGGAATTACGGCCCCTTTGACTACTGGGGC
CAGGGAACCCTGGTCACCGTCTCCTCA
HCVR (V_{H}) Amino Acid Sequence
(SEQ ID NO: 565)
EVQLVESGGGLVQPGRSLTLSCAASGFTFDNFGMHWVRQGPGKGLEWVSGLTWNS
GVIGYADSVKGRFTISRDNAKNSLYLQMNSLRPEDTALYYCAKDIRNYGPFDYWG
QGTLVTVSS
HCDR1:
(SEQ ID NO: 566)
GFTFDNFG
HCDR2:
(SEQ ID NO: 567)
LTWNSGVI
HCDR3:
(SEQ ID NO: 568)
AKDIRNYGPFDY
LCVR (V_{L}) Nucleotide Sequence
(SEQ ID NO: 569)
GAAATTGTGTTGACGCAGTCTCCAGGCACCCTGTCTTTGTCTCCAGGGGAAAGAG
CCACCCTCTCCTGCAGGGCCAGTCAGAGTGTTAGCAGCAGCTACTTAGCCTGGTA
CCAGCAGAAACCTGGCCAGGCTCCCAGGCTCCTCATCTATGGTGCATCCAGCAGG
GCCACTGGCATCCCAGACAGGTTCAGTGGCAGTGGGTCTGGGACAGACTTCACTC
TCACCATCAGCAGACTGGAGCCTGAAGATTTTGCAGTGTATTACTGTCAGCAGTA
TGGTAGCTCACCTTGGACGTTCGGCCAAGGGACCAAGGTGGAAATCAAA
LCVR (V_{L}) Amino Acid Sequence
(SEQ ID NO: 570)
EIVLTQSPGTLSLSPGERATLSCRASQSVSSSYLAWYQQKPGQAPRLLIYGASSR
ATGIPDRFSGSGSGTDFTLTISRLEPEDFAVYYCQQYGSSPWTFGQGTKVEIK
LCDR1:
(SEQ ID NO: 571)
QSVSSSY
LCDR2:
(SEQ ID NO: 572)
GAS
LCDR3:
(SEQ ID NO: 573)
QQYGSSPWT
12843B (REGN17075 anti-hTfR scFv;
REGN16824 anti-hTfR scFv: hGAA;
REGN17081 anti-hTfR Fab)
HCVR (V_{H}) Nucleotide Sequence
(SEQ ID NO: 574)
GAGGTGCAGCTGGTGGAGTCTGGGGGAGGCTTAGTACAGCCTGGAGGGTCCCTAA
GACTCTCCTGTGCAGCCTCTGGATTCACCTTCAATATTTTTGAAATGAACTGGGT
CCGCCAGGCTCCAGGGAAGGGGCTGGAGTGGATTTCCTACATTAGTAGTCGTGGA
ACTACCACATACTACGCAGACTCTGTGAGGGGCCGATTCACCATCTCCAGAGACA
ACGCCAAGAACTCACTGTATCTGCAAATGAACAGCCTGAGAGCCGAGGACACGGC
TGTTTATTACTGTGCGAGAGATTATGAAGCAACAATCCCTTTTGACTTCTGGGGC
CAGGGAACCCTGGTCACCGTCTCCTCA
HCVR (V_{H}) Amino Acid Sequence
(SEQ ID NO: 575)
EVQLVESGGGLVQPGGSLRLSCAASGFTFNIFEMNWVRQAPGKGLEWISYISSRG
TTTYYADSVRGRFTISRDNAKNSLYLQMNSLRAEDTAVYYCARDYEATIPFDFWG
QGTLVTVSS
HCDR1:
(SEQ ID NO: 576)
GFTFNIFE
HCDR2:
(SEQ ID NO: 577)
ISSRGTTT
HCDR3:
(SEQ ID NO: 578)
ARDYEATIPFDF
LCVR (V_{L}) Nucleotide Sequence
(SEQ ID NO: 579)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 580)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 581)
QSISSY
LCDR2:
(SEQ ID NO: 582)
AAS
LCDR3:
(SEQ ID NO: 583)
QQSYSTPPIT
12844B
HCVR (V_{H}) Nucleotide Sequence
(SEQ ID NO: 584)
GAGGTGCAGCTGGTGGAGTCTGGGGGAAGTGTGGTACGGCCTGGGGGGTCCCTGA
GACTCTCCTGTGAAGCCTCTGGATTCACCTTTGATGATTATGGCATGAGCTGGGT
CCGCCAAGATCCAGGGAAGGGGCTGGAGTGGGTCTCTGGTATTAATTGGAATGGT
GATAGAACAAATTATGCAGACTCTGTGAAGGGCCGATTCATCATTTCCAGAGACA
ACGCCAAGAACTCTGTGTATCTACAAATGAACAGTCTGAGAGCGGAGGACTCGGC
CTTGTATCACTGTGCGAGAGATCAGGGACTCGGAGTGGCAGCTACCCTTGACTAC
TGGGGCCAGGGAACCCTGGTCACCGTCTCCTCA
HCVR (V_{H}) Amino Acid Sequence
(SEQ ID NO: 585)
EVQLVESGGSVVRPGGSLRLSCEASGFTFDDYGMSWVRQDPGKGLEWVSGINWNG
DRTNYADSVKGRFIISRDNAKNSVYLQMNSLRAEDSALYHCARDQGLGVAATLDY
WGQGTLVTVSS
HCDR1:
(SEQ ID NO: 586)
GFTFDDYG
HCDR2:
(SEQ ID NO: 587)
INWNGDRT
HCDR3:
(SEQ ID NO: 588)
ARDQGLGVAATLDY
LCVR (V_{L}) Nucleotide Sequence
(SEQ ID NO: 589)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 590)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 591)
QSISSY
LCDR2:
(SEQ ID NO: 592)
AAS
LCDR3:
(SEQ ID NO: 593)
QQSYSTPPIT
12845B (REGN17082 Fab; REGN17076 scFv;
REGN16825 anti-hTfR scFv: hGAA)
HCVR (V_{H}) Nucleotide Sequence
(SEQ ID NO: 594)
GAGGTGCAGCTGGTGGAGTCTGGGGGAGGCTTGGTACAGCCTGGAGGGTCCCTGA
GACTCTCCTGTGCAGCCTCTGGATTCACCGTCAGTAATTATGAAATGAACTGGGT
CCGCCAGGCTCCAGGGAAGGGGCTGGAGTGGGTTTCATACATTAGTAGTAGTACC
AGTAACATATACTACGCAGACTCTGTGAAGGGCCGATTCACCATCTCCAGAGACA
ACGCCGAGAACTCACTGTATCTGCAGATGAACAGCCTGAGAGTCGAGGACACGGC
TGTTTATTACTGTGTGAGAGATGGGATTGTAGTAGTTCCAGTTGGTCGTGGATAC
TACTATTACGGTTTGGACGTCTGGGGCCAAGGGACCACGGTCACCGTCTCCTCA
HCVR (V_{H}) Amino Acid Sequence
(SEQ ID NO: 595)
EVQLVESGGGLVQPGGSLRLSCAASGFTVSNYEMNWVRQAPGKGLEWVSYISSST
SNIYYADSVKGRFTISRDNAENSLYLQMNSLRVEDTAVYYCVRDGIVVVPVGRGY
YYYGLDVWGQGTTVTVSS
HCDR1:
(SEQ ID NO: 596)
GFTVSNYE
HCDR2:
(SEQ ID NO: 597)
ISSSTSNI
HCDR3:
(SEQ ID NO: 598)
VRDGIVVVPVGRGYYYYGLDV
LCVR (V_{L}) Nucleotide Sequence
(SEQ ID NO: 599)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 600)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 601)
QSISSY
LCDR2:
(SEQ ID NO: 602)
AAS
LCDR3:
(SEQ ID NO: 603)
QQSYSTPPIT
12839B (REGN17080 Fab; REGN17074 scFv;
REGN16822 anti-hTfR scFv: hGAA)
HCVR (V_{H}) Nucleotide Sequence
(SEQ ID NO: 604)
CAGGTGCAGCTGGTGGAGTCTGGGGGAGGCGTGGTCCAGCCTGGAAGGTCCCTGA
GACTCTCCTGCGCAGCCTCTGGATTCCCCTTTAGTAATTATGTCATGTATTGGGT
CCGCCAGGCTCCAGGCAAGGGGCTGGAGTGGGTGGCTCTTATTTTTTTTGACGGA
AAGAAAAACTATCATGCAGACTCCGTGAAGGGCCGATTCACCATAACCAGAGACA
ATTCCAAAAATATGTTATATCTGCAAATGAACAGCCTGAGACCTGAGGACGCGGC
TGTGTATTACTGTGCGAAAATCCATTGTCCTAATGGTGTATGTTACAAGGGGTAT
TACGGAATGGACGTCTGGGGCCAAGGGACCACGGTCACCGTCTCCTCA
HCVR (V_{H}) Amino Acid Sequence
(SEQ ID NO: 605)
QVQLVESGGGVVQPGRSLRLSCAASGFPFSNYVMYWVRQAPGKGLEWVALIFFDG
KKNYHADSVKGRFTITRDNSKNMLYLQMNSLRPEDAAVYYCAKIHCPNGVCYKGY
YGMDVWGQGTTVTVSS
HCDR1:
(SEQ ID NO: 606)
GFPFSNYV
HCDR2:
(SEQ ID NO: 607)
IFFDGKKN
HCDR3:
(SEQ ID NO: 608)
AKIHCPNGVCYKGYYGMDV
LCVR (V_{L}) Nucleotide Sequence
(SEQ ID NO: 609)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 610)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 611)
QSISSY
LCDR2:
(SEQ ID NO: 612)
AAS
LCDR3:
(SEQ ID NO: 613)
QQSYSTPPIT
12841B (REGN16823 anti-hTfR scFv: hGAA)
HCVR (V_{H}) Nucleotide Sequence
(SEQ ID NO: 614)
GAGGTGCAGCTGGTGGAGTCTGGGGGAGGCTTGGTCCAGCCTGGGGGGTCCCTAA
GACTCTCCTGTGCAGCCTCTGGATTCACCTTTAGTAACTATTGGATGAACTGGGT
CCGCCAGGCTCCAGGGAAGGGACTGGAGTGGGTGGCCAATATAAAAGAAGATGGA
GGTAAGAAATTGTATGTGGACTCTGTGAAGGGCCGATTCACCATCTCCAGAGACA
ACGCCAAGAACTCACTGTTTCTGCAAATGAACAGCCTGAGAGCCGAGGACACGGC
TGTGTATTATTGTGCGAGAGAAGATACAACTTTGGTTGTGGACTACTACTACTAC
GGTATGGACGTCTGGGGCCAAGGGACCACGGTCACCGTCTCCTCA
HCVR (V_{H}) Amino Acid Sequence
(SEQ ID NO: 615)
EVQLVESGGGLVQPGGSLRLSCAASGFTFSNYWMNWVRQAPGKGLEWVANIKEDG
GKKLYVDSVKGRFTISRDNAKNSLFLQMNSLRAEDTAVYYCAREDTTLVVDYYYY
GMDVWGQGTTVTVSS
HCDR1:
(SEQ ID NO: 616)
GFTFSNYW
HCDR2:
(SEQ ID NO: 617)
IKEDGGKK
HCDR3:
(SEQ ID NO: 618)
AREDTTLVVDYYYYGMDV
LCVR (V_{L}) Nucleotide Sequence
(SEQ ID NO: 619)
GACATCCAGATGACCCAGTCTCCATCCTCCCTGTCTGCATCTGTAGGAGACAGAG
TCACCATCACTTGCCGGGCAAGTCAGAGCATTAGCAGCTATTTAAATTGGTATCA
GCAGAAACCAGGGAAAGCCCCTAAGCTCCTGATCTATGCTGCATCCAGTTTGCAA
AGTGGGGTCCCGTCAAGGTTCAGTGGCAGTGGATCTGGGACAGATTTCACTCTCA
CCATCAGCAGTCTGCAACCTGAAGATTTTGCAACTTACTACTGTCAACAGAGTTA
CAGTACCCCTCCGATCACCTTCGGCCAAGGGACACGACTGGAGATTAAA
LCVR (V_{L}) Amino Acid Sequence
(SEQ ID NO: 620)
DIQMTQSPSSLSASVGDRVTITCRASQSISSYLNWYQQKPGKAPKLLIYAASSLQ
SGVPSRFSGSGSGTDFTLTISSLQPEDFATYYCQQSYSTPPITFGQGTRLEIK
LCDR1:
(SEQ ID NO: 621)
QSISSY
LCDR2:
(SEQ ID NO: 622)
AAS
LCDR3:
(SEQ ID NO: 623)
QQSYSTPPIT
12850B (REGN16828 anti-hTfR scFv: hGAA)
HCVR (V_{H}) Nucleotide Sequence
(SEQ ID NO: 624)
CAGGTCCAGCTGGTGCAGTCTGGGGCTGAGGTGAAGAAGCCTGGGTCCTCGGTGA
AGGTCTCCTGCAAGGCTTCTGGAGGCACCTTCAACACCTATGCTATCACCTGGGT
GCGACAGGCCCCTGGACAAGGGCTTGAATGGATGGGGGGAATCATCCCTATCTCT
GGCATAGCAGAGTACGCACAGAAGTTCCAGGGCAGAGTCACGATCACCACGGATG
ACTCCTCGACCACAGCCTACATGGAACTGAACAGTCTGAGATCTGAGGACACGGC
CGTGTATTACTGTGCGAGCTGGAACTACGCACTCTACTACTTCTACGGTATGGAC
GTCTGGGGCCGAGGGACCACGGTCACCGTCTCCTCA
HCVR (V_{H}) Amino Acid Sequence
(SEQ ID NO: 625)
QVQLVQSGAEVKKPGSSVKVSCKASGGTFNTYAITWVRQAPGQGLEWMGGIIPIS
GIAEYAQKFQGRVTITTDDSSTTAYMELNSLRSEDTAVYYCASWNYALYYFYGMD
VWGRGTTVTVSS
HCDR1:
(SEQ ID NO: 626)
GGTFNTYA
HCDR2:
(SEQ ID NO: 627)
IIPISGIA
HCDR3:
(SEQ ID NO: 628)
ASWNYALYYFYGMDV
LCVR (V_{L}) Nucleotide Sequence
(SEQ ID NO: 629)
GAAATTGTGTTGACGCAGTCTCCAGGCACCCTGTCTTTGTCTCCAGGGGAAAGAG
CCACCCTCTCCTGCAGGGCCAGTCAGAGTGTTAGCAGCAGCTACTTAGCCTGGTA
CCAGCAGAAACCTGGCCAGGCTCCCAGGCTCCTCATCTATGGTGCATCCAGCAGG
GCCACTGGCATCCCAGACAGGTTCAGTGGCAGTGGGTCTGGGACAGACTTCACTC
TCACCATCAGCAGACTGGAGCCTGAAGATTTTGCAGTGTATTACTGTCAGCAGTA
TGGTAGCTCACCTTGGACGTTCGGCCAAGGGACCAAGGTGGAAATCAAA
LCVR (V_{L}) Amino Acid Sequence
(SEQ ID NO: 630)
EIVLTQSPGTLSLSPGERATLSCRASQSVSSSYLAWYQQKPGQAPRLLIYGASSR
ATGIPDRFSGSGSGTDFTLTISRLEPEDFAVYYCQQYGSSPWTFGQGTKVEIK
LCDR1:
(SEQ ID NO: 631)
QSVSSSY
LCDR2:
(SEQ ID NO: 632)
GAS
LCDR3:
(SEQ ID NO: 633)
QQYGSSPWT
69261
HCVR (V_{H}) Nucleotide Sequence
(SEQ ID NO: 634)
CAGGTGCAGCTGGTGGAGTCTGGGGGAGGCTTGGTCAAGCCTGGAGGGTCCCTGA
GACTCTCCTGTGCAGCCTCTGGATTCACCTTCAGTGTCTATTACATGAACTGGAT
CCGCCAGGCTCCAGGGAAGGGCCTGGAGTGGGTTTCATACATTAGTAGTAGTGGT
AGTACCATATACTACGCAGACTCTGTGAAGGGCCGATTCACCATCTCCAGGGACA
ACGCCAAGAACTCACTGTATCTCCAAATGAACAGTCTGAGAGCCGAGGACACGGC
CGTATATTACTGTGGGAGAGAAGGGTATAGTGGGACTTATTCTTATTACGGTATG
GACGTCTGGGGCCAAGGGACCACGGTCACCGTCTCCTCA
HCVR (V_{H}) Amino Acid Sequence
(SEQ ID NO: 635)
QVQLVESGGGLVKPGGSLRLSCAASGFTFSVYYMNWIRQAPGKGLEWVSYISSSG
STIYYADSVKGRFTISRDNAKNSLYLQMNSLRAEDTAVYYCGREGYSGTYSYYGM
DVWGQGTTVTVSS
HCDR1:
(SEQ ID NO: 636)
GFTFSVYY
HCDR2:
(SEQ ID NO: 637)
ISSSGSTI
HCDR3:
(SEQ ID NO: 638)
GREGYSGTYSYYGMDV
LCVR (V_{L}) Nucleotide Sequence
(SEQ ID NO: 639)
GATATTGTGATGACTCAGTCTCCACTCTCCCTGCCCGTCACCCCTGGAGAGCCGG
CCTCCATCTCCTGCAGGTCTAGTCAGAGCCTCCTGCATAGTAATGGATACAACTA
TTTGGATTGGTACCTGCAGAAGCCAGGGCAGTCTCCACAGTTCCTGATCTATTTG
GGTTCTAATCGGGCCTCCGGGGTCCCTGACAGGTTCAGTGGCAGTGGATCAGGCA
CAGATTTTACACTGAAAATCAACAGAGTGGAGGCTGAGGATGTTGGGGTTTATTA
CTGCATGCAAGCTCTACAAACTCCGTACACTTTTGGCCAGGGGACCAAGCTGGAG
ATCAAA
LCVR (V_{L}) Amino Acid Sequence
(SEQ ID NO: 640)
DIVMTQSPLSLPVTPGEPASISCRSSQSLLHSNGYNYLDWYLQKPGQSPQFLIYL
GSNRASGVPDRFSGSGSGTDFTLKINRVEAEDVGVYYCMQALQTPYTFGQGTKLE
IK
LCDR1:
(SEQ ID NO: 641)
QSLLHSNGYNY
LCDR2:
(SEQ ID NO: 642)
LGS
LCDR3:
(SEQ ID NO: 643)
MQALQTPYT
69263
HCVR (V_{H}) Nucleotide Sequence
(SEQ ID NO: 644)
GAAGTGCAGCTGGTGGAGTCTGGGGGAGGGTTGGTACAGCCTGGCAGGTCCCTGA
GACTCTCCTGTGCAGTCTCTGGATTCACCTTTGATGATTATGCCATGCACTGGGT
CCGGCAAGCTCCAGGGAAGGGCCTGGAGTGGGTCTCAGGTATTAGTTGGAATAGT
GGTACCAGAGGATATGCGGACTCTGTGAAGGGCCGATTCACCATCTCCAGAGACA
ACGCCAAGAACTCCCTGTATCTGCAAATGAACAGTCTGAGAGGTGAGGACACGGC
CTTGTATTACTGTGTAAAAGATATTACGATATCCCCCAACTACTACGGTATGGAC
GTCTGGGGCCAAGGGACCACGGTCACCGTCTCCTCA
HCVR (V_{H}) Amino Acid Sequence
(SEQ ID NO: 645)
EVQLVESGGGLVQPGRSLRLSCAVSGFTFDDYAMHWVRQAPGKGLEWVSGISWNS
GTRGYADSVKGRFTISRDNAKNSLYLQMNSLRGEDTALYYCVKDITISPNYYGMD
VWGQGTTVTVSS
HCDR1:
(SEQ ID NO: 646)
GFTFDDYA
HCDR2:
(SEQ ID NO: 647)
ISWNSGTR
HCDR3:
(SEQ ID NO: 648)
VKDITISPNYYGMDV
LCVR (V_{L}) Nucleotide Sequence
(SEQ ID NO: 649)
GACATCCAGATGACCCAGTCTCCATCCTCCCTGTCTGCATCTGTAGGAGACAGAG
TCACCATCACTTGCCGGGCGAGTCAGGACATTAGCCATTATTCAGCCTGGTATCA
GCAGAAACCAGGGAAACTTCCTAACCTCCTGATCTATGCTGCATCCACTTTGCAA
TCAGGGGTCCCATCTCGGTTCAGTGGCAGTGGATCTGGGACAGATTTCTCTCTCA
CCACCAGCAGCCTGCAGCCTGAAGATGTTGCAACTTATTACTGTCAAAAGTATAA
CAGTGTCCCTCTCACTTTCGGCGGAGGGACCAAGGTGGAGATCAAA
LCVR (V_{L}) Amino Acid Sequence
(SEQ ID NO: 650)
DIQMTQSPSSLSASVGDRVTITCRASQDISHYSAWYQQKPGKLPNLLIYAASTLQ
SGVPSRFSGSGSGTDFSLTTSSLQPEDVATYYCQKYNSVPLTFGGGTKVEIK
LCDR1:
(SEQ ID NO: 651)
QDISHY
LCDR2:
(SEQ ID NO: 652)
AAS
LCDR3:
(SEQ ID NO: 653)
QKYNSVPLT

In some multidomain therapeutic proteins, the TfR-binding delivery domain is an antibody, an antibody fragment or other antigen-binding protein. In some multidomain therapeutic proteins, the TfR-binding delivery domain is an antigen-binding protein. Examples of antigen-binding proteins include, for example, a receptor-fusion molecule, a trap molecule, a receptor-Fc fusion molecule, an antibody, an Fab fragment, an F(ab′)2 fragment, an Fd fragment, an Fv fragment, a single-chain Fv (scFv) molecule, a dAb fragment, an isolated complementarity determining region (CDR), a CDR3 peptide, a constrained FR3-CDR3-FR4 peptide, a domain-specific antibody, a single domain antibody, a domain-deleted antibody, a chimeric antibody, a CDR-grafted antibody, a diabody, a triabody, a tetrabody, a minibody, a nanobody, a monovalent nanobody, a bivalent nanobody, a small modular immunopharmaceutical (SMIP), a camelid antibody (VHH heavy chain homodimeric antibody), and a shark variable IgNAR domain.

Provided herein are antibodies that bind specifically to the human transferrin receptor 1. The term “antibody,” as used herein, refers to immunoglobulin molecules comprising four polypeptide chains, two heavy chains (HCs) and two light chains (LCs), inter-connected by disulfide bonds. In an embodiment, each antibody heavy chain (HC) comprises a heavy chain variable region (“HCVR” or “V_{H}”) (e.g., comprising SEQ ID NO: 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, and/or 645 or a variant thereof) and a heavy chain constant region (e.g., human IgG, human IgG1 or human IgG4); and each antibody light chain (LC) comprises a light chain variable region (“LCVR or “V_{L}”) (e.g., SEQ ID NO: 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, and/or 650 or a variant thereof) and a light chain constant region (e.g., human kappa or human lambda). The V_{H} and V_{L} regions can be further subdivided into regions of hypervariability, termed complementarity determining regions (CDR), interspersed with regions that are more conserved, termed framework regions (FR). Each V_{H} and V_{L} comprises three CDRs and four FRs. Anti-TfR antibodies disclosed herein can also be fused to GAA.

An anti-TfR antigen-binding protein of the present invention may be an antigen-binding fragment of an antibody which may be tethered to GAA. The terms “antigen-binding portion” or “antigen-binding fragment” of an antibody, as used herein, refers to an immunoglobulin molecule that binds antigen but that does not include all of the sequences of a full antibody (preferably, the full antibody is an IgG). Non-limiting examples of antigen-binding fragments include: (i) Fab fragments; (ii) F(ab′)2 fragments; (iii) Fd fragments; (iv) Fv fragments; (v) single-chain Fv (scFv) molecules; and (vi) dAb fragments; consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies and small modular immunopharmaceuticals (SMIPs), are also encompassed within the expression “antigen-binding fragment,” as used herein.

An anti-TfR antigen-binding protein may be an scFv which may be tethered to a GAA. An scFv (single chain fragment variable) has variable regions of heavy (V_{H}) and light (V_{L}) domains (in either order), which, preferably, are joined together by a flexible linker (e.g., peptide linker). The length of the flexible linker used to link both of the V regions may be important for yielding the correct folding of the polypeptide chain. Previously, it has been estimated that the peptide linker must span 3.5 nm (35 Å) between the carboxy terminus of the variable domain and the amino terminus of the other domain without affecting the ability of the domains to fold and form an intact antigen-binding site (Huston et al., Protein engineering of single-chain Fv analogs and fusion proteins. Methods in Enzymology. 1991; 203:46-88). In an embodiment, the linker comprises an amino acid sequence of such length to separate the variable domains by about 3.5 nm.

An antigen-binding fragment of an antibody will, in an embodiment, comprise at least one variable domain. The variable domain may be of any size or amino acid composition and will generally comprise at least one CDR, which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

“Isolated” antigen-binding proteins (e.g., antibodies or antigen-binding fragments thereof), polypeptides, polynucleotides and vectors, are at least partially free of other biological molecules from the cells or cell culture from which they are produced. Such biological molecules include nucleic acids, proteins, other antibodies or antigen-binding fragments, lipids, carbohydrates, or other material such as cellular debris and growth medium. An isolated antigen-binding protein may further be at least partially free of expression system components such as biological molecules from a host cell or of the growth medium thereof. Generally, the term “isolated” is not intended to refer to a complete absence of such biological molecules (e.g., minor or insignificant amounts of impurity may remain) or to an absence of water, buffers, or salts or to components of a pharmaceutical formulation that includes the antigen-binding proteins (e.g., antibodies or antigen-binding fragments).

An anti-TfR antigen-binding protein of the present invention may be a monoclonal antibody or an antigen-binding fragment of a monoclonal antibody which may be tethered to GAA. The present invention includes monoclonal anti-TfR antigen-binding proteins, e.g., antibodies and antigen-binding fragments thereof, as well as monoclonal compositions comprising a plurality of isolated monoclonal antigen-binding proteins. The term “monoclonal antibody” or “mAb,” as used herein, refers to a member of a population of substantially homogeneous antibodies, i.e., the antibody molecules comprising the population are identical in amino acid sequence except for possible naturally occurring mutations that may be present in minor amounts. A “plurality” of such monoclonal antibodies and fragments in a composition refers to a concentration of identical (i.e., as discussed above, in amino acid sequence except for possible naturally occurring mutations that may be present in minor amounts) antibodies and fragments which is above that which would normally occur in nature, e.g., in the blood of a host organism such as a mouse or a human.

In an embodiment, an anti-TfR antigen-binding protein, e.g., antibody or antigen-binding fragment (which may be tethered to a Payload) comprises a heavy chain constant domain, e.g., of the type IgA (e.g., IgA1 or IgA2), IgD, IgE, IgG (e.g., IgG1, IgG2, IgG3 and IgG4) or IgM. In an embodiment of the invention, an antigen-binding protein, e.g., antibody or antigen-binding fragment, comprises a light chain constant domain, e.g., of the type kappa or lambda.

Included herein are human anti-TfR antigen-binding proteins which may be tethered to GAA. The term “human” antigen-binding protein, such as an antibody or antigen-binding fragment, as used herein, includes antibodies and fragments having variable and constant regions derived from human germline immunoglobulin sequences whether in a human cell or grafted into a non-human cell, e.g., a mouse cell. See, e.g., U.S. Pat. Nos. 8,502,018, 6,596,541 or 5,789,215. The anti-TfR human mAbs of the invention may include amino acid residues not encoded by human germline immunoglobulin sequences (e.g., mutations introduced by random or site-specific mutagenesis in vitro or by somatic mutation in vivo), for example, in the CDRs and in particular CDR3. However, the term “human antibody,” as used herein, is not intended to include mAbs in which CDR sequences derived from the germline of another mammalian species (e.g., mouse) have been grafted onto human FR sequences. The term includes antibodies recombinantly produced in a non-human mammal or in cells of a non-human mammal. The term is not intended to include natural antibodies directly isolated from a human subject.

Also included herein are anti-TfR chimeric antigen-binding proteins, e.g., antibodies and antigen-binding fragments thereof (which may be tethered to GAA), and methods of use thereof. As used herein, a “chimeric antibody” is an antibody having the variable domain from a first antibody and the constant domain from a second antibody, where the first and second antibodies are from different species. (see, e.g., U.S. Pat. No. 4,816,567; and Morrison et al., (1984) Proc. Natl. Acad. Sci. USA 81: 6851-6855).

The term “recombinant” anti-TfR antigen-binding proteins, such as antibodies or antigen-binding fragments thereof (which may be tethered to GAA), refers to such molecules created, expressed, isolated or obtained by technologies or methods known in the art as recombinant DNA technology which include, e.g., DNA splicing and transgenic expression. The term includes antibodies expressed in a non-human mammal (including transgenic non-human mammals, e.g., transgenic mice), or a cell (e.g., CHO cells) such as a cellular expression system or isolated from a recombinant combinatorial human antibody library.

A “variant” of a polypeptide refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein (e.g., any of SEQ ID NOs: 335-338; 340-343; 345-348; 350-353; 355-358; 360-363; 365-368; 370-373; 375-378; 380-383; 385-388; 390-393; 395-398; 400-403; 405-408; 410-413; 415-418; 420-423; 425-428; 430-433; 435-438; 440-443; 445-448; 450-453; 455-458; 460-463; 465-468; 470-473; 475-478; 480-483; 485-488; 490-493; 495-498; 500-503; 505-508; 510-513; 515-518; 520-523; 525-528; 530-533; 535-538; 540-543; 545-548; 550-553; 555-558; 560-563; 565-568; 570-573; 575-578; 580-583; 585-588; 590-593; 595-598; 600-603; 605-608; 610-613; 615-618; 620-623; 625-628; 630-633; 635-638; 640-643; 645-648; 650-653; 821 (optionally not including the N-terminal MHRPRRRGTRPPPLALLAALLLAARGADA (SEQ ID NO: 791)), 822 (optionally not including the N-terminal MHRPRRRGTRPPPLALLAALLLAARGADA (SEQ ID NO: 791)), 823 (optionally not including the N-terminal MHRPRRRGTRPPPLALLAALLLAARGADA (SEQ ID NO: 791)), 824 (optionally not including the N-terminal MHRPRRRGTRPPPLALLAALLLAARGADA (SEQ ID NO: 791)); 656-691, 721-790, or 793-820); when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment) and/or comprising the amino acid sequence but having one or more (e.g., 1, 2, 3, 4, 5, 6, 7, 8, 9 or 10) mutations (e.g., point mutation, insertion, truncation, and/or deletion). Preferably, functional GAA ectodomain.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

In an embodiment of the invention, an anti-hTfR:Payload or anti-hTfR:Payload (e.g., in scFv, Fab, antibody or antigen-binding fragment thereof format), e.g., wherein the Payload is human GAA, exhibits one or more of the following characteristics:
Affinity (K_{D}) for binding to human TfR at 25° C. in surface plasmon resonance format of about 41 nM or a higher affinity (e.g., about 1 or 0.1 nM or about 0.18 to about 1.2 nM, or higher);
Affinity (K_{D}) for binding to monkey TfR at 25° C. in surface plasmon resonance format of about 0 nM (no detectable binding) or a higher affinity (e.g., about 20 nM or higher);
Ratio of K_{D} for binding to monkey TfR/human TfR at 25° C. in surface plasmon resonance format of from 0 to 278 (e.g., about 17 or 18);
Blocks about 3, 5, 10 or 13% hTfR (e.g., Hmm-hTFRC such as REGN2431) binding to Human Holo-Tf when in Fab format (IgG1), e.g., no more than about 45% blocking;
Blocks about 6, 8, 10 or 13% hTfR (e.g., Hmm-hTFRC such as REGN2431) binding to Human Holo-Tf when in scFv (V_{K}-V_{H}) format, e.g., no more than about 45% blocking;
Blocks about 11, 17, 23 or 26% hTfR (e.g., Hmm-hTFRC such as REGN2431) binding to Human Holo-Tf when in scFv (V_{H}-V_{L}) format, e.g., no more than about 45% blocking;
Exhibits a ratio of about 1 or greater; 0.67 or greater; 1.08 or greater; 0.91 or greater; 0.65 or greater; 0.55 or greater; 0.50 or greater; 0.27 or greater; 0.72 or greater; 1.05 or greater; 0.49 or greater; 0.29 or greater; 1.29 or greater; 1.72 or greater; 1.79 or greater; 3.08 or greater; 1.24 or greater; 0.59 or greater; or 0.47 or greater (or about 1-2 or greater) mature hGAA protein in brain (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain of humans administered said scFv:hGAA molecule;
Exhibits a ratio of about 0.44, 0.05, 1.13 or 0.60 (about 0.1-1.2) mature hGAA protein in brain parenchyma (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the brain parenchyma of humans administered said scFv:hGAA molecule;
Exhibits a ratio of about 0.67, 1.80, 1.78 or 7.74 (about 1-2) mature hGAA protein in quadriceps (normalized to that of positive control 8D3:GAA scFv) in mice (e.g., Tfrc^{hum/hum} knock-in mice) administered the molecule via HDD, when in anti-hTfR scFv:hGAA format; or delivers mature human GAA protein to the quadricep or other muscle tissue of humans administered said scFv:hGAA molecule;
Exhibits a ratio of about 0.94, 0.49, 0.61 or 1.90 (about 0.1-1.2) mature hGAA protein in brain parenchyma (normalized to that of positive control 8D3:GAA scFv) in mice (e.g., Tfrc^{hum} knock-in mice) administered the molecule via AAV8 liver depot, when in anti-hTfR scFv:hGAA format; or delivers mature human GAA protein to the brain parenchyma of humans administered said scFv:hGAA molecule via viral, e.g., AAV, liver depot or parenterally delivered in protein scFv:hGAA fusion format;
Delivers mature hGAA protein to serum, liver, cerebrum, cerebellum, spinal cord, heart and/or quadricep in mice (e.g., Tfrc^{hum} knock-in mice) administered the molecule via AAV8 liver depot, when in anti-hTfR scFv:hGAA format; or delivers mature human GAA protein to the serum, liver, cerebrum, cerebellum, spinal cord, heart and/or quadricep of humans administered said scFv:hGAA molecule via viral, e.g., AAV, liver depot or parenterally delivered in protein scFv:hGAA fusion format;
Reduces glycogen stored in cerebrum, cerebellum, spinal cord, heart and/or quadricep in mice (e.g., Tfrc^{hum} knock-in mice) administered the molecule via AAV8 liver depot, when in anti-hTfR scFv:hGAA format; e.g., by at least 75% to greater than 95% or greater than 99%; or reduces glycogen stored in cerebrum, cerebellum, spinal cord, heart and/or quadricep of humans administered said scFv:hGAA molecule via viral, e.g., AAV, liver depot, or parenterally delivered in protein scFv:hGAA fusion format;
Reduces glycogen levels in tissues (e.g., cerebellum) of Gaa^{-/-}/Tfrc^{hum} mice treated with liver-depot AAV8 anti-hTFRC scfv:hGAA (e.g., 4e11 vg/kg AAV8) by at least about 90% (e.g., about 95% or more) relative to untreated Gaa^{-/-}/Tfrc^{hum} mice;
Reduces glycogen levels in tissues (e.g., quadricep) of Gaa^{-/-}/Tfrc^{hum} mice treated with liver-depot AAV8 anti-hTFRC scfv:hGAA (e.g., 4e11 vg/kg AAV8) by at least about 89% (e.g., about 90% or 91% or more) relative to untreated Gaa^{-/-}/Tfrc^{hum} mice; or of humans treated with the fusion, e.g., by parenteral deliver of the fusion protein;
Does not cause abnormal iron homeostasis when administered (e.g., by HDD or AAV8 episomal liver depot) to Tfrch^{hum} mice; e.g., wherein the mice maintain normal serum, heart, liver and/or spleen iron levels, normal total iron-binding capacity (TIBC), and/or normal hepcidin levels); or when administered to humans, e.g., by parenteral deliver of the fusion protein;
When chromosomally inserted (e.g., into the albumin gene locus) or delivered episomally to a subject (e.g., to a human or Gaa^{-/-}/Tfrc^{hum/hum} mouse), for example, in an AAV8 vector, DNA encoding the fusion causes expression of mature human GAA to serum, liver, cerebrum and/or quadricep; and/or
When chromosomally inserted (e.g., into the albumin gene locus) or delivered episomally (e.g., to a human or Gaa^{-/-}/Tfrc^{hum/hum} mouse), for example, in an AAV8 vector, DNA encoding the fusion reduces glycogen levels in the cerebrum and/or quadricep.
Tfrc^{hum} or Tfrc^{hum/hum} are homozygous knock-in mice.

The amino acid sequences of domains in anti-human transferrin receptor antigen-binding proteins of fusions disclosed herein are summarized below in Table 3. For example, anti-human transferrin receptor 1 antibodies and antigen-binding fragments thereof (e.g., scFvs and Fabs) comprising the HCVR and LCVR of the molecules in Table 3; or comprising the CDRs thereof, fused to GAA, are disclosed herein.

As discussed, an anti-hTfR:GAA scFv fusion protein (e.g., 31874B; 31863B; 69348; 69340; 69331; 69332; 69326; 69329; 69323; 69305; 69307; 12795B; 12798B; 12799B; 12801B; 12802B; 12808B; 12812B; 12816B; 12833B; 12834B; 12835B; 12847B; 12848B; 12843B; 12844B; 12845B; 12839B; 12841B; 12850B; 69261; or 69263) comprises an optional signal peptide, connected to an scFv (e.g., including a V_{L} and a V_{H} optionally connected by a linker), connected to an option linker, connected to a GAA. For example, the optional signal peptide can be the signal peptide from Mus musculus Ror1 (e.g., consisting of the amino acids MHRPRRRGTRPPPLALLAALLLAARGADA (SEQ ID NO: 791).

In a particular multidomain therapeutic protein, the TfR-binding delivery domain is an anti-TfR scFv. For example, the scFv can include a V_{L} and a V_{H} optionally connected by a linker.

In one example, the anti-hTfR scFv can comprise: (i) a heavy chain variable region that comprises the HCDR1, HCDR2 and HCDR3 of a HCVR comprising the amino acid sequence set forth in SEQ ID NO: 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, or 645; and/or (ii) a light chain variable region that comprises the LCDR1, LCDR2 and LCDR3 of a LCVR comprising the amino acid sequence set forth in SEQ ID NO: 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, or 650.

In another example, the anti-TfR scFv can comprise: (1) a HCVR comprising the HCDR1, HCDR2 and HCDR3 of a HCVR that comprises the amino acid sequence set forth in SEQ ID NO: 335 (or a variant thereof); and a LCVR comprising the LCDR1, LCDR2 and LCDR3 of a LCVR that comprises the amino acid sequence set forth in SEQ ID NO: 340 (or a variant thereof); (2) a HCVR comprising the HCDR1, HCDR2 and HCDR3 of a HCVR that comprises the amino acid sequence set forth in SEQ ID NO: 345 (or a variant thereof); and a LCVR comprising the LCDR1, LCDR2 and LCDR3 of a LCVR that comprises the amino acid sequence set forth in SEQ ID NO: 350 (or a variant thereof); (3) a HCVR comprising the HCDR1, HCDR2 and HCDR3 of a HCVR that comprises the amino acid sequence set forth in SEQ ID NO: 355 (or a variant thereof); and a LCVR comprising the LCDR1, LCDR2 and LCDR3 of a LCVR that comprises the amino acid sequence set forth in SEQ ID NO: 360 (or a variant thereof); (4) a HCVR comprising the HCDR1, HCDR2 and HCDR3 of a HCVR that comprises the amino acid sequence set forth in SEQ ID NO: 365 (or a variant thereof); and a LCVR comprising the LCDR1, LCDR2 and LCDR3 of a LCVR that comprises the amino acid sequence set forth in SEQ ID NO: 370 (or a variant thereof); (5) a HCVR comprising the HCDR1, HCDR2 and HCDR3 of a HCVR that comprises the amino acid sequence set forth in SEQ ID NO: 375 (or a variant thereof); and a LCVR comprising the LCDR1, LCDR2 and LCDR3 of a LCVR that comprises the amino acid sequence set forth in SEQ ID NO: 380 (or a variant thereof); (6) a HCVR comprising the HCDR1, HCDR2 and HCDR3 of a HCVR that comprises the amino acid sequence set forth in SEQ ID NO: 385 (or a variant thereof); and a LCVR comprising the LCDR1, LCDR2 and LCDR3 of a LCVR that comprises the amino acid sequence set forth in SEQ ID NO: 390 (or a variant thereof); (7) a HCVR comprising the HCDR1, HCDR2 and HCDR3 of a HCVR that comprises the amino acid sequence set forth in SEQ ID NO: 395 (or a variant thereof); and a LCVR comprising the LCDR1, LCDR2 and LCDR3 of a LCVR that comprises the amino acid sequence set forth in SEQ ID NO: 400 (or a variant thereof); (8) a HCVR comprising the HCDR1, HCDR2 and HCDR3 of a HCVR that comprises the amino acid sequence set forth in SEQ ID NO: 405 (or a variant thereof); and a LCVR comprising the LCDR1, LCDR2 and LCDR3 of a LCVR that comprises the amino acid sequence set forth in SEQ ID NO: 410 (or a variant thereof); (9) a HCVR comprising the HCDR1, HCDR2 and HCDR3 of a HCVR that comprises the amino acid sequence set forth in SEQ ID NO: 415 (or a variant thereof); and a LCVR comprising the LCDR1, LCDR2 and LCDR3 of a LCVR that comprises the amino acid sequence set forth in SEQ ID NO: 420 (or a variant thereof); (10) a HCVR comprising the HCDR1, HCDR2 and HCDR3 of a HCVR that comprises the amino acid sequence set forth in SEQ ID NO: 425 (or a variant thereof); and a LCVR comprising the LCDR1, LCDR2 and LCDR3 of a LCVR that comprises the amino acid sequence set forth in SEQ ID NO: 430 (or a variant thereof); (11) a HCVR comprising the HCDR1, HCDR2 and HCDR3 of a HCVR that comprises the amino acid sequence set forth in SEQ ID NO: 435 (or a variant thereof); and a LCVR comprising the LCDR1, LCDR2 and LCDR3 of a LCVR that comprises the amino acid sequence set forth in SEQ ID NO: 440 (or a variant thereof); (12) a HCVR comprising the HCDR1, HCDR2 and HCDR3 of a HCVR that comprises the amino acid sequence set forth in SEQ ID NO: 445 (or a variant thereof); and a LCVR comprising the LCDR1, LCDR2 and LCDR3 of a LCVR that comprises the amino acid sequence set forth in SEQ ID NO: 450 (or a variant thereof); (13) a HCVR comprising the HCDR1, HCDR2 and HCDR3 of a HCVR that comprises the amino acid sequence set forth in SEQ ID NO: 455 (or a variant thereof); and a LCVR comprising the LCDR1, LCDR2 and LCDR3 of a LCVR that comprises the amino acid sequence set forth in SEQ ID NO: 460 (or a variant thereof); (14) a HCVR comprising the HCDR1, HCDR2 and HCDR3 of a HCVR that comprises the amino acid sequence set forth in SEQ ID NO: 465 (or a variant thereof); and a LCVR comprising the LCDR1, LCDR2 and LCDR3 of a LCVR that comprises the amino acid sequence set forth in SEQ ID NO: 470 (or a variant thereof); (15) a HCVR comprising the HCDR1, HCDR2 and HCDR3 of a HCVR that comprises the amino acid sequence set forth in SEQ ID NO: 475 (or a variant thereof); and a LCVR comprising the LCDR1, LCDR2 and LCDR3 of a LCVR that comprises the amino acid sequence set forth in SEQ ID NO: 480 (or a variant thereof); (16) a HCVR comprising the HCDR1, HCDR2 and HCDR3 of a HCVR that comprises the amino acid sequence set forth in SEQ ID NO: 485 (or a variant thereof); and a LCVR comprising the LCDR1, LCDR2 and LCDR3 of a LCVR that comprises the amino acid sequence set forth in SEQ ID NO: 490 (or a variant thereof); (17) a HCVR comprising the HCDR1, HCDR2 and HCDR3 of a HCVR that comprises the amino acid sequence set forth in SEQ ID NO: 495 (or a variant thereof); and a LCVR comprising the LCDR1, LCDR2 and LCDR3 of a LCVR that comprises the amino acid sequence set forth in SEQ ID NO: 500 (or a variant thereof); (18) a HCVR comprising the HCDR1, HCDR2 and HCDR3 of a HCVR that comprises the amino acid sequence set forth in SEQ ID NO: 505 (or a variant thereof); and a LCVR comprising the LCDR1, LCDR2 and LCDR3 of a LCVR that comprises the amino acid sequence set forth in SEQ ID NO: 510 (or a variant thereof); (19) a HCVR comprising the HCDR1, HCDR2 and HCDR3 of a HCVR that comprises the amino acid sequence set forth in SEQ ID NO: 515 (or a variant thereof); and a LCVR comprising the LCDR1, LCDR2 and LCDR3 of a LCVR that comprises the amino acid sequence set forth in SEQ ID NO: 520 (or a variant thereof); (20) a HCVR comprising the HCDR1, HCDR2 and HCDR3 of a HCVR that comprises the amino acid sequence set forth in SEQ ID NO: 525 (or a variant thereof); and a LCVR comprising the LCDR1, LCDR2 and LCDR3 of a LCVR that comprises the amino acid sequence set forth in SEQ ID NO: 530 (or a variant thereof); (21) a HCVR comprising the HCDR1, HCDR2 and HCDR3 of a HCVR that comprises the amino acid sequence set forth in SEQ ID NO: 535 (or a variant thereof); and a LCVR comprising the LCDR1, LCDR2 and LCDR3 of a LCVR that comprises the amino acid sequence set forth in SEQ ID NO: 540 (or a variant thereof); (22) a HCVR comprising the HCDR1, HCDR2 and HCDR3 of a HCVR that comprises the amino acid sequence set forth in SEQ ID NO: 545 (or a variant thereof); and a LCVR comprising the LCDR1, LCDR2 and LCDR3 of a LCVR that comprises the amino acid sequence set forth in SEQ ID NO: 550 (or a variant thereof); (23) a HCVR comprising the HCDR1, HCDR2 and HCDR3 of a HCVR that comprises the amino acid sequence set forth in SEQ ID NO: 555 (or a variant thereof); and a LCVR comprising the LCDR1, LCDR2 and LCDR3 of a LCVR that comprises the amino acid sequence set forth in SEQ ID NO: 560 (or a variant thereof); (24) a HCVR comprising the HCDR1, HCDR2 and HCDR3 of a HCVR that comprises the amino acid sequence set forth in SEQ ID NO: 565 (or a variant thereof); and a LCVR comprising the LCDR1, LCDR2 and LCDR3 of a LCVR that comprises the amino acid sequence set forth in SEQ ID NO: 570 (or a variant thereof); (25) a HCVR comprising the HCDR1, HCDR2 and HCDR3 of a HCVR that comprises the amino acid sequence set forth in SEQ ID NO: 575 (or a variant thereof); and a LCVR comprising the LCDR1, LCDR2 and LCDR3 of a LCVR that comprises the amino acid sequence set forth in SEQ ID NO: 580 (or a variant thereof); (26) a HCVR comprising the HCDR1, HCDR2 and HCDR3 of a HCVR that comprises the amino acid sequence set forth in SEQ ID NO: 585 (or a variant thereof); and a LCVR comprising the LCDR1, LCDR2 and LCDR3 of a LCVR that comprises the amino acid sequence set forth in SEQ ID NO: 590 (or a variant thereof); (27) a HCVR comprising the HCDR1, HCDR2 and HCDR3 of a HCVR that comprises the amino acid sequence set forth in SEQ ID NO: 595 (or a variant thereof); and a LCVR comprising the LCDR1, LCDR2 and LCDR3 of a LCVR that comprises the amino acid sequence set forth in SEQ ID NO: 600 (or a variant thereof); (28) a HCVR comprising the HCDR1, HCDR2 and HCDR3 of a HCVR that comprises the amino acid sequence set forth in SEQ ID NO: 605 (or a variant thereof); and a LCVR comprising the LCDR1, LCDR2 and LCDR3 of a LCVR that comprises the amino acid sequence set forth in SEQ ID NO: 610 (or a variant thereof); (29) a HCVR comprising the HCDR1, HCDR2 and HCDR3 of a HCVR that comprises the amino acid sequence set forth in SEQ ID NO: 615 (or a variant thereof); and a LCVR comprising the LCDR1, LCDR2 and LCDR3 of a LCVR that comprises the amino acid sequence set forth in SEQ ID NO: 620 (or a variant thereof); (30) a HCVR comprising the HCDR1, HCDR2 and HCDR3 of a HCVR that comprises the amino acid sequence set forth in SEQ ID NO: 625 (or a variant thereof); and a LCVR comprising the LCDR1, LCDR2 and LCDR3 of a LCVR that comprises the amino acid sequence set forth in SEQ ID NO: 630 (or a variant thereof); (31) a HCVR comprising the HCDR1, HCDR2 and HCDR3 of a HCVR that comprises the amino acid sequence set forth in SEQ ID NO: 635 (or a variant thereof); and a LCVR comprising the LCDR1, LCDR2 and LCDR3 of a LCVR that comprises the amino acid sequence set forth in SEQ ID NO: 640 (or a variant thereof); or (32) a HCVR comprising the HCDR1, HCDR2 and HCDR3 of a HCVR that comprises the amino acid sequence set forth in SEQ ID NO: 645 (or a variant thereof); and a LCVR comprising the LCDR1, LCDR2 and LCDR3 of a LCVR that comprises the amino acid sequence set forth in SEQ ID NO: 650 (or a variant thereof). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR scFv can comprise: (a) a HCVR that comprises: an HCDR1 comprising the amino acid sequence set forth in SEQ ID NO: 336 (or a variant thereof), an HCDR2 comprising the amino acid sequence set forth in SEQ ID NO: 337 (or a variant thereof), and an HCDR3 comprising the amino acid sequence set forth in SEQ ID NO: 338 (or a variant thereof); and a LCVR that comprises: an LCDR1 comprising the amino acid sequence set forth in SEQ ID NO: 341 (or a variant thereof), an LCDR2 comprising the amino acid sequence set forth in SEQ ID NO: 342 (or a variant thereof), and an LCDR3 comprising the amino acid sequence set forth in SEQ ID NO: 343 (or a variant thereof); (b) a HCVR that comprises: an HCDR1 comprising the amino acid sequence set forth in SEQ ID NO: 346 (or a variant thereof), an HCDR2 comprising the amino acid sequence set forth in SEQ ID NO: 347 (or a variant thereof), and an HCDR3 comprising the amino acid sequence set forth in SEQ ID NO: 348 (or a variant thereof); and a LCVR that comprises: an LCDR1 comprising the amino acid sequence set forth in SEQ ID NO: 351 (or a variant thereof), an LCDR2 comprising the amino acid sequence set forth in SEQ ID NO: 352 (or a variant thereof), and an LCDR3 comprising the amino acid sequence set forth in SEQ ID NO: 353 (or a variant thereof); (c) a HCVR that comprises: an HCDR1 comprising the amino acid sequence set forth in SEQ ID NO: 356 (or a variant thereof), an HCDR2 comprising the amino acid sequence set forth in SEQ ID NO: 357 (or a variant thereof), and an HCDR3 comprising the amino acid sequence set forth in SEQ ID NO: 358 (or a variant thereof); and a LCVR that comprises: an LCDR1 comprising the amino acid sequence set forth in SEQ ID NO: 361 (or a variant thereof), an LCDR2 comprising the amino acid sequence set forth in SEQ ID NO: 362 (or a variant thereof), and an LCDR3 comprising the amino acid sequence set forth in SEQ ID NO: 363 (or a variant thereof); (d) a HCVR that comprises: an HCDR1 comprising the amino acid sequence set forth in SEQ ID NO: 366 (or a variant thereof), an HCDR2 comprising the amino acid sequence set forth in SEQ ID NO: 367 (or a variant thereof), and an HCDR3 comprising the amino acid sequence set forth in SEQ ID NO: 368 (or a variant thereof); and a LCVR that comprises: an LCDR1 comprising the amino acid sequence set forth in SEQ ID NO: 371 (or a variant thereof), an LCDR2 comprising the amino acid sequence set forth in SEQ ID NO: 372 (or a variant thereof), and an LCDR3 comprising the amino acid sequence set forth in SEQ ID NO: 373 (or a variant thereof); (e) a HCVR that comprises: an HCDR1 comprising the amino acid sequence set forth in SEQ ID NO: 376 (or a variant thereof), an HCDR2 comprising the amino acid sequence set forth in SEQ ID NO: 377 (or a variant thereof), and an HCDR3 comprising the amino acid sequence set forth in SEQ ID NO: 378 (or a variant thereof); and a LCVR that comprises: an LCDR1 comprising the amino acid sequence set forth in SEQ ID NO: 381 (or a variant thereof), an LCDR2 comprising the amino acid sequence set forth in SEQ ID NO: 382 (or a variant thereof), and an LCDR3 comprising the amino acid sequence set forth in SEQ ID NO: 383 (or a variant thereof); (f) a HCVR that comprises: an HCDR1 comprising the amino acid sequence set forth in SEQ ID NO: 386 (or a variant thereof), an HCDR2 comprising the amino acid sequence set forth in SEQ ID NO: 387 (or a variant thereof), and an HCDR3 comprising the amino acid sequence set forth in SEQ ID NO: 388 (or a variant thereof); and a LCVR that comprises: an LCDR1 comprising the amino acid sequence set forth in SEQ ID NO: 391 (or a variant thereof), an LCDR2 comprising the amino acid sequence set forth in SEQ ID NO: 392 (or a variant thereof), and an LCDR3 comprising the amino acid sequence set forth in SEQ ID NO: 393 (or a variant thereof); (g) a HCVR that comprises: an HCDR1 comprising the amino acid sequence set forth in SEQ ID NO: 396 (or a variant thereof), an HCDR2 comprising the amino acid sequence set forth in SEQ ID NO: 397 (or a variant thereof), and an HCDR3 comprising the amino acid sequence set forth in SEQ ID NO: 398 (or a variant thereof); and a LCVR that comprises: an LCDR1 comprising the amino acid sequence set forth in SEQ ID NO: 401 (or a variant thereof), an LCDR2 comprising the amino acid sequence set forth in SEQ ID NO: 402 (or a variant thereof), and an LCDR3 comprising the amino acid sequence set forth in SEQ ID NO: 403 (or a variant thereof); (h) a HCVR that comprises: an HCDR1 comprising the amino acid sequence set forth in SEQ ID NO: 406 (or a variant thereof), an HCDR2 comprising the amino acid sequence set forth in SEQ ID NO: 407 (or a variant thereof), and an HCDR3 comprising the amino acid sequence set forth in SEQ ID NO: 408 (or a variant thereof); and a LCVR that comprises: an LCDR1 comprising the amino acid sequence set forth in SEQ ID NO: 411 (or a variant thereof), an LCDR2 comprising the amino acid sequence set forth in SEQ ID NO: 412 (or a variant thereof), and an LCDR3 comprising the amino acid sequence set forth in SEQ ID NO: 413 (or a variant thereof); (i) a HCVR that comprises: an HCDR1 comprising the amino acid sequence set forth in SEQ ID NO: 416 (or a variant thereof), an HCDR2 comprising the amino acid sequence set forth in SEQ ID NO: 417 (or a variant thereof), and an HCDR3 comprising the amino acid sequence set forth in SEQ ID NO: 418 (or a variant thereof); and a LCVR that comprises: an LCDR1 comprising the amino acid sequence set forth in SEQ ID NO: 421 (or a variant thereof), an LCDR2 comprising the amino acid sequence set forth in SEQ ID NO: 422 (or a variant thereof), and an LCDR3 comprising the amino acid sequence set forth in SEQ ID NO: 423 (or a variant thereof); (j) a HCVR that comprises: an HCDR1 comprising the amino acid sequence set forth in SEQ ID NO: 426 (or a variant thereof), an HCDR2 comprising the amino acid sequence set forth in SEQ ID NO: 427 (or a variant thereof), and an HCDR3 comprising the amino acid sequence set forth in SEQ ID NO: 428 (or a variant thereof); and a LCVR that comprises: an LCDR1 comprising the amino acid sequence set forth in SEQ ID NO: 431 (or a variant thereof), an LCDR2 comprising the amino acid sequence set forth in SEQ ID NO: 432 (or a variant thereof), and an LCDR3 comprising the amino acid sequence set forth in SEQ ID NO: 433 (or a variant thereof); (k) a HCVR that comprises: an HCDR1 comprising the amino acid sequence set forth in SEQ ID NO: 436 (or a variant thereof), an HCDR2 comprising the amino acid sequence set forth in SEQ ID NO: 437 (or a variant thereof), and an HCDR3 comprising the amino acid sequence set forth in SEQ ID NO: 438 (or a variant thereof); and a LCVR that comprises: an LCDR1 comprising the amino acid sequence set forth in SEQ ID NO: 441 (or a variant thereof), an LCDR2 comprising the amino acid sequence set forth in SEQ ID NO: 442 (or a variant thereof), and an LCDR3 comprising the amino acid sequence set forth in SEQ ID NO: 443 (or a variant thereof); (1) a HCVR that comprises: an HCDR1 comprising the amino acid sequence set forth in SEQ ID NO: 446 (or a variant thereof), an HCDR2 comprising the amino acid sequence set forth in SEQ ID NO: 447 (or a variant thereof), and an HCDR3 comprising the amino acid sequence set forth in SEQ ID NO: 448 (or a variant thereof); and a LCVR that comprises: an LCDR1 comprising the amino acid sequence set forth in SEQ ID NO: 451 (or a variant thereof), an LCDR2 comprising the amino acid sequence set forth in SEQ ID NO: 452 (or a variant thereof), and an LCDR3 comprising the amino acid sequence set forth in SEQ ID NO: 453 (or a variant thereof); (m) a HCVR that comprises: an HCDR1 comprising the amino acid sequence set forth in SEQ ID NO: 456 (or a variant thereof), an HCDR2 comprising the amino acid sequence set forth in SEQ ID NO: 457 (or a variant thereof), and an HCDR3 comprising the amino acid sequence set forth in SEQ ID NO: 458 (or a variant thereof); and a LCVR that comprises: an LCDR1 comprising the amino acid sequence set forth in SEQ ID NO: 461 (or a variant thereof), an LCDR2 comprising the amino acid sequence set forth in SEQ ID NO: 462 (or a variant thereof), and an LCDR3 comprising the amino acid sequence set forth in SEQ ID NO: 463 (or a variant thereof); (n) a HCVR that comprises: an HCDR1 comprising the amino acid sequence set forth in SEQ ID NO: 466 (or a variant thereof), an HCDR2 comprising the amino acid sequence set forth in SEQ ID NO: 467 (or a variant thereof), and an HCDR3 comprising the amino acid sequence set forth in SEQ ID NO: 468 (or a variant thereof); and a LCVR that comprises: an LCDR1 comprising the amino acid sequence set forth in SEQ ID NO: 471 (or a variant thereof), an LCDR2 comprising the amino acid sequence set forth in SEQ ID NO: 472 (or a variant thereof), and an LCDR3 comprising the amino acid sequence set forth in SEQ ID NO: 473 (or a variant thereof); (o) a HCVR that comprises: an HCDR1 comprising the amino acid sequence set forth in SEQ ID NO: 476 (or a variant thereof), an HCDR2 comprising the amino acid sequence set forth in SEQ ID NO: 477 (or a variant thereof), and an HCDR3 comprising the amino acid sequence set forth in SEQ ID NO: 478 (or a variant thereof); and a LCVR that comprises: an LCDR1 comprising the amino acid sequence set forth in SEQ ID NO: 481 (or a variant thereof), an LCDR2 comprising the amino acid sequence set forth in SEQ ID NO: 482 (or a variant thereof), and an LCDR3 comprising the amino acid sequence set forth in SEQ ID NO: 483 (or a variant thereof); (p) a HCVR that comprises: an HCDR1 comprising the amino acid sequence set forth in SEQ ID NO: 486 (or a variant thereof), an HCDR2 comprising the amino acid sequence set forth in SEQ ID NO: 487 (or a variant thereof), and an HCDR3 comprising the amino acid sequence set forth in SEQ ID NO: 488 (or a variant thereof); and a LCVR that comprises: an LCDR1 comprising the amino acid sequence set forth in SEQ ID NO: 491 (or a variant thereof), an LCDR2 comprising the amino acid sequence set forth in SEQ ID NO: 492 (or a variant thereof), and an LCDR3 comprising the amino acid sequence set forth in SEQ ID NO: 493 (or a variant thereof); (q) a HCVR that comprises: an HCDR1 comprising the amino acid sequence set forth in SEQ ID NO: 496 (or a variant thereof), an HCDR2 comprising the amino acid sequence set forth in SEQ ID NO: 497 (or a variant thereof), and an HCDR3 comprising the amino acid sequence set forth in SEQ ID NO: 498 (or a variant thereof); and a LCVR that comprises: an LCDR1 comprising the amino acid sequence set forth in SEQ ID NO: 501 (or a variant thereof), an LCDR2 comprising the amino acid sequence set forth in SEQ ID NO: 502 (or a variant thereof), and an LCDR3 comprising the amino acid sequence set forth in SEQ ID NO: 503 (or a variant thereof); (r) a HCVR that comprises: an HCDR1 comprising the amino acid sequence set forth in SEQ ID NO: 506 (or a variant thereof), an HCDR2 comprising the amino acid sequence set forth in SEQ ID NO: 507 (or a variant thereof), and an HCDR3 comprising the amino acid sequence set forth in SEQ ID NO: 508 (or a variant thereof); and a LCVR that comprises: an LCDR1 comprising the amino acid sequence set forth in SEQ ID NO: 511 (or a variant thereof), an LCDR2 comprising the amino acid sequence set forth in SEQ ID NO: 512 (or a variant thereof), and an LCDR3 comprising the amino acid sequence set forth in SEQ ID NO: 513 (or a variant thereof); (s) a HCVR that comprises: an HCDR1 comprising the amino acid sequence set forth in SEQ ID NO: 516 (or a variant thereof), an HCDR2 comprising the amino acid sequence set forth in SEQ ID NO: 517 (or a variant thereof), and an HCDR3 comprising the amino acid sequence set forth in SEQ ID NO: 518 (or a variant thereof); and a LCVR that comprises: an LCDR1 comprising the amino acid sequence set forth in SEQ ID NO: 521 (or a variant thereof), an LCDR2 comprising the amino acid sequence set forth in SEQ ID NO: 522 (or a variant thereof), and an LCDR3 comprising the amino acid sequence set forth in SEQ ID NO: 523 (or a variant thereof); (t) a HCVR that comprises: an HCDR1 comprising the amino acid sequence set forth in SEQ ID NO: 526 (or a variant thereof), an HCDR2 comprising the amino acid sequence set forth in SEQ ID NO: 527 (or a variant thereof), and an HCDR3 comprising the amino acid sequence set forth in SEQ ID NO: 528 (or a variant thereof); and a LCVR that comprises: an LCDR1 comprising the amino acid sequence set forth in SEQ ID NO: 531 (or a variant thereof), an LCDR2 comprising the amino acid sequence set forth in SEQ ID NO: 532 (or a variant thereof), and an LCDR3 comprising the amino acid sequence set forth in SEQ ID NO: 533 (or a variant thereof); (u) a HCVR that comprises: an HCDR1 comprising the amino acid sequence set forth in SEQ ID NO: 536 (or a variant thereof), an HCDR2 comprising the amino acid sequence set forth in SEQ ID NO: 537 (or a variant thereof), and an HCDR3 comprising the amino acid sequence set forth in SEQ ID NO: 538 (or a variant thereof); and a LCVR that comprises: an LCDR1 comprising the amino acid sequence set forth in SEQ ID NO: 541 (or a variant thereof), an LCDR2 comprising the amino acid sequence set forth in SEQ ID NO: 542 (or a variant thereof), and an LCDR3 comprising the amino acid sequence set forth in SEQ ID NO: 543 (or a variant thereof); (v) a HCVR that comprises: an HCDR1 comprising the amino acid sequence set forth in SEQ ID NO: 546 (or a variant thereof), an HCDR2 comprising the amino acid sequence set forth in SEQ ID NO: 547 (or a variant thereof), and an HCDR3 comprising the amino acid sequence set forth in SEQ ID NO: 548 (or a variant thereof); and a LCVR that comprises: an LCDR1 comprising the amino acid sequence set forth in SEQ ID NO: 551 (or a variant thereof), an LCDR2 comprising the amino acid sequence set forth in SEQ ID NO: 552 (or a variant thereof), and an LCDR3 comprising the amino acid sequence set forth in SEQ ID NO: 553 (or a variant thereof); (w) a HCVR that comprises: an HCDR1 comprising the amino acid sequence set forth in SEQ ID NO: 556 (or a variant thereof), an HCDR2 comprising the amino acid sequence set forth in SEQ ID NO: 557 (or a variant thereof), and an HCDR3 comprising the amino acid sequence set forth in SEQ ID NO: 558 (or a variant thereof); and a LCVR that comprises: an LCDR1 comprising the amino acid sequence set forth in SEQ ID NO: 561 (or a variant thereof), an LCDR2 comprising the amino acid sequence set forth in SEQ ID NO: 562 (or a variant thereof), and an LCDR3 comprising the amino acid sequence set forth in SEQ ID NO: 563 (or a variant thereof); (x) a HCVR that comprises: an HCDR1 comprising the amino acid sequence set forth in SEQ ID NO: 566 (or a variant thereof), an HCDR2 comprising the amino acid sequence set forth in SEQ ID NO: 567 (or a variant thereof), and an HCDR3 comprising the amino acid sequence set forth in SEQ ID NO: 568 (or a variant thereof); and a LCVR that comprises: an LCDR1 comprising the amino acid sequence set forth in SEQ ID NO: 571 (or a variant thereof), an LCDR2 comprising the amino acid sequence set forth in SEQ ID NO: 572 (or a variant thereof), and an LCDR3 comprising the amino acid sequence set forth in SEQ ID NO: 573 (or a variant thereof); (y) a HCVR that comprises: an HCDR1 comprising the amino acid sequence set forth in SEQ ID NO: 576 (or a variant thereof), an HCDR2 comprising the amino acid sequence set forth in SEQ ID NO: 577 (or a variant thereof), and an HCDR3 comprising the amino acid sequence set forth in SEQ ID NO: 578 (or a variant thereof); and a LCVR that comprises: an LCDR1 comprising the amino acid sequence set forth in SEQ ID NO: 581 (or a variant thereof), an LCDR2 comprising the amino acid sequence set forth in SEQ ID NO: 582 (or a variant thereof), and an LCDR3 comprising the amino acid sequence set forth in SEQ ID NO: 583 (or a variant thereof); (z) a HCVR that comprises: an HCDR1 comprising the amino acid sequence set forth in SEQ ID NO: 586 (or a variant thereof), an HCDR2 comprising the amino acid sequence set forth in SEQ ID NO: 587 (or a variant thereof), and an HCDR3 comprising the amino acid sequence set forth in SEQ ID NO: 588 (or a variant thereof); and a LCVR that comprises: an LCDR1 comprising the amino acid sequence set forth in SEQ ID NO: 591 (or a variant thereof), an LCDR2 comprising the amino acid sequence set forth in SEQ ID NO: 592 (or a variant thereof), and an LCDR3 comprising the amino acid sequence set forth in SEQ ID NO: 593 (or a variant thereof); (aa) a HCVR that comprises: an HCDR1 comprising the amino acid sequence set forth in SEQ ID NO: 596 (or a variant thereof), an HCDR2 comprising the amino acid sequence set forth in SEQ ID NO: 597 (or a variant thereof), and an HCDR3 comprising the amino acid sequence set forth in SEQ ID NO: 598 (or a variant thereof); and a LCVR that comprises: an LCDR1 comprising the amino acid sequence set forth in SEQ ID NO: 601 (or a variant thereof), an LCDR2 comprising the amino acid sequence set forth in SEQ ID NO: 602 (or a variant thereof), and an LCDR3 comprising the amino acid sequence set forth in SEQ ID NO: 603 (or a variant thereof); (ab) a HCVR that comprises: an HCDR1 comprising the amino acid sequence set forth in SEQ ID NO: 606 (or a variant thereof), an HCDR2 comprising the amino acid sequence set forth in SEQ ID NO: 607 (or a variant thereof), and an HCDR3 comprising the amino acid sequence set forth in SEQ ID NO: 608 (or a variant thereof); and a LCVR that comprises: an LCDR1 comprising the amino acid sequence set forth in SEQ ID NO: 611 (or a variant thereof), an LCDR2 comprising the amino acid sequence set forth in SEQ ID NO: 612 (or a variant thereof), and an LCDR3 comprising the amino acid sequence set forth in SEQ ID NO: 613 (or a variant thereof); (ac) a HCVR that comprises: an HCDR1 comprising the amino acid sequence set forth in SEQ ID NO: 616 (or a variant thereof), an HCDR2 comprising the amino acid sequence set forth in SEQ ID NO: 617 (or a variant thereof), and an HCDR3 comprising the amino acid sequence set forth in SEQ ID NO: 618 (or a variant thereof); and a LCVR that comprises: an LCDR1 comprising the amino acid sequence set forth in SEQ ID NO: 621 (or a variant thereof), an LCDR2 comprising the amino acid sequence set forth in SEQ ID NO: 622 (or a variant thereof), and an LCDR3 comprising the amino acid sequence set forth in SEQ ID NO: 623 (or a variant thereof); (ad) a HCVR that comprises: an HCDR1 comprising the amino acid sequence set forth in SEQ ID NO: 626 (or a variant thereof), an HCDR2 comprising the amino acid sequence set forth in SEQ ID NO: 627 (or a variant thereof), and an HCDR3 comprising the amino acid sequence set forth in SEQ ID NO: 628 (or a variant thereof); and a LCVR that comprises: an LCDR1 comprising the amino acid sequence set forth in SEQ ID NO: 631 (or a variant thereof), an LCDR2 comprising the amino acid sequence set forth in SEQ ID NO: 632 (or a variant thereof), and an LCDR3 comprising the amino acid sequence set forth in SEQ ID NO: 633 (or a variant thereof); (ae) a HCVR that comprises: an HCDR1 comprising the amino acid sequence set forth in SEQ ID NO: 636 (or a variant thereof), an HCDR2 comprising the amino acid sequence set forth in SEQ ID NO: 637 (or a variant thereof), and an HCDR3 comprising the amino acid sequence set forth in SEQ ID NO: 638 (or a variant thereof); and a LCVR that comprises: an LCDR1 comprising the amino acid sequence set forth in SEQ ID NO: 641 (or a variant thereof), an LCDR2 comprising the amino acid sequence set forth in SEQ ID NO: 642 (or a variant thereof), and an LCDR3 comprising the amino acid sequence set forth in SEQ ID NO: 643 (or a variant thereof); and/or (af) a HCVR that comprises: an HCDR1 comprising the amino acid sequence set forth in SEQ ID NO: 646 (or a variant thereof), an HCDR2 comprising the amino acid sequence set forth in SEQ ID NO: 647 (or a variant thereof), and an HCDR3 comprising the amino acid sequence set forth in SEQ ID NO: 648 (or a variant thereof); and a LCVR that comprises: an LCDR1 comprising the amino acid sequence set forth in SEQ ID NO: 651 (or a variant thereof), an LCDR2 comprising the amino acid sequence set forth in SEQ ID NO: 652 (or a variant thereof), and an LCDR3 comprising the amino acid sequence set forth in SEQ ID NO: 653 (or a variant thereof). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

In another example, the anti-TfR scFv can comprise: (i) a HCVR that comprises the amino acid sequence set forth in SEQ ID NO: 335 (or a variant thereof); and a LCVR that comprises the amino acid sequence set forth in SEQ ID NO: 340 (or a variant thereof); (ii) a HCVR that comprises the amino acid sequence set forth in SEQ ID NO: 345 (or a variant thereof); and a LCVR that comprises the amino acid sequence set forth in SEQ ID NO: 350 (or a variant thereof); (iii) a HCVR that comprises the amino acid sequence set forth in SEQ ID NO: 355 (or a variant thereof); and a LCVR that comprises the amino acid sequence set forth in SEQ ID NO: 360 (or a variant thereof); (iv) a HCVR that comprises the amino acid sequence set forth in SEQ ID NO: 365 (or a variant thereof); and a LCVR that comprises the amino acid sequence set forth in SEQ ID NO: 370 (or a variant thereof); (v) a HCVR that comprises the amino acid sequence set forth in SEQ ID NO: 375 (or a variant thereof); and a LCVR that comprises the amino acid sequence set forth in SEQ ID NO: 380 (or a variant thereof); (vi) a HCVR that comprises the amino acid sequence set forth in SEQ ID NO: 385 (or a variant thereof); and a LCVR that comprises the amino acid sequence set forth in SEQ ID NO: 390 (or a variant thereof); (vii) a HCVR that comprises the amino acid sequence set forth in SEQ ID NO: 395 (or a variant thereof); and a LCVR that comprises the amino acid sequence set forth in SEQ ID NO: 400 (or a variant thereof); (viii) a HCVR that comprises the amino acid sequence set forth in SEQ ID NO: 405 (or a variant thereof); and a LCVR that comprises the amino acid sequence set forth in SEQ ID NO: 410 (or a variant thereof); (ix) a HCVR that comprises the amino acid sequence set forth in SEQ ID NO: 415 (or a variant thereof); and a LCVR that comprises the amino acid sequence set forth in SEQ ID NO: 420 (or a variant thereof); (x) a HCVR that comprises the amino acid sequence set forth in SEQ ID NO: 425 (or a variant thereof); and a LCVR that comprises the amino acid sequence set forth in SEQ ID NO: 430 (or a variant thereof); (xi) a HCVR that comprises the amino acid sequence set forth in SEQ ID NO: 435 (or a variant thereof); and a LCVR that comprises the amino acid sequence set forth in SEQ ID NO: 440 (or a variant thereof); (xii) a HCVR that comprises the amino acid sequence set forth in SEQ ID NO: 445 (or a variant thereof); and a LCVR that comprises the amino acid sequence set forth in SEQ ID NO: 450 (or a variant thereof); (xiii) a HCVR that comprises the amino acid sequence set forth in SEQ ID NO: 455 (or a variant thereof); and a LCVR that comprises the amino acid sequence set forth in SEQ ID NO: 460 (or a variant thereof); (xiv) a HCVR that comprises the amino acid sequence set forth in SEQ ID NO: 465 (or a variant thereof); and a LCVR that comprises the amino acid sequence set forth in SEQ ID NO: 470 (or a variant thereof); (xv) a HCVR that comprises the amino acid sequence set forth in SEQ ID NO: 475 (or a variant thereof); and a LCVR that comprises the amino acid sequence set forth in SEQ ID NO: 480 (or a variant thereof); (xvi) a HCVR that comprises the amino acid sequence set forth in SEQ ID NO: 485 (or a variant thereof); and a LCVR that comprises the amino acid sequence set forth in SEQ ID NO: 490 (or a variant thereof); (xvii) a HCVR that comprises the amino acid sequence set forth in SEQ ID NO: 495 (or a variant thereof); and a LCVR that comprises the amino acid sequence set forth in SEQ ID NO: 500 (or a variant thereof); (xviii) a HCVR that comprises the amino acid sequence set forth in SEQ ID NO: 505 (or a variant thereof); and a LCVR that comprises the amino acid sequence set forth in SEQ ID NO: 510 (or a variant thereof); (xix) a HCVR that comprises the amino acid sequence set forth in SEQ ID NO: 515 (or a variant thereof); and a LCVR that comprises the amino acid sequence set forth in SEQ ID NO: 520 (or a variant thereof); (xx) a HCVR that comprises the amino acid sequence set forth in SEQ ID NO: 525 (or a variant thereof); and a LCVR that comprises the amino acid sequence set forth in SEQ ID NO: 530 (or a variant thereof); (xxi) a HCVR that comprises the amino acid sequence set forth in SEQ ID NO: 535 (or a variant thereof); and a LCVR that comprises the amino acid sequence set forth in SEQ ID NO: 540 (or a variant thereof); (xxii) a HCVR that comprises the amino acid sequence set forth in SEQ ID NO: 545 (or a variant thereof); and a LCVR that comprises the amino acid sequence set forth in SEQ ID NO: 550 (or a variant thereof); (xxiii) a HCVR that comprises the amino acid sequence set forth in SEQ ID NO: 555 (or a variant thereof); and a LCVR that comprises the amino acid sequence set forth in SEQ ID NO: 560 (or a variant thereof); (xxiv) a HCVR that comprises the amino acid sequence set forth in SEQ ID NO: 565 (or a variant thereof); and a LCVR that comprises the amino acid sequence set forth in SEQ ID NO: 570 (or a variant thereof); (xxv) a HCVR that comprises the amino acid sequence set forth in SEQ ID NO: 575 (or a variant thereof); and a LCVR that comprises the amino acid sequence set forth in SEQ ID NO: 580 (or a variant thereof); (xxvi) a HCVR that comprises the amino acid sequence set forth in SEQ ID NO: 585 (or a variant thereof); and a LCVR that comprises the amino acid sequence set forth in SEQ ID NO: 590 (or a variant thereof); (xxvii) a HCVR that comprises the amino acid sequence set forth in SEQ ID NO: 595 (or a variant thereof); and a LCVR that comprises the amino acid sequence set forth in SEQ ID NO: 600 (or a variant thereof); (xxviii) a HCVR that comprises the amino acid sequence set forth in SEQ ID NO: 605 (or a variant thereof); and a LCVR that comprises the amino acid sequence set forth in SEQ ID NO: 610 (or a variant thereof); (xxix) a HCVR that comprises the amino acid sequence set forth in SEQ ID NO: 615 (or a variant thereof); and a LCVR that comprises the amino acid sequence set forth in SEQ ID NO: 620 (or a variant thereof); (xxx) a HCVR that comprises the amino acid sequence set forth in SEQ ID NO: 625 (or a variant thereof); and a LCVR that comprises the amino acid sequence set forth in SEQ ID NO: 630 (or a variant thereof); (xxxi) a HCVR that comprises the amino acid sequence set forth in SEQ ID NO: 635 (or a variant thereof); and a LCVR that comprises the amino acid sequence set forth in SEQ ID NO: 640 (or a variant thereof); and/or (xxxii) a HCVR that comprises the amino acid sequence set forth in SEQ ID NO: 645 (or a variant thereof); and a LCVR that comprises the amino acid sequence set forth in SEQ ID NO: 650 (or a variant thereof), optionally wherein the HCVR and LCVR are linked by a linker (e.g., that comprises an amino acid sequence, e.g., about 10 amino acids in length, for example, 1, 2, 3, 4, 5, 6, 7, 8, 8, or 10 repeats of Gly₄Ser (SEQ ID NO: 718). A variant refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a referenced amino acid sequence that is set forth herein.

Examples of polynucleotides encoding anti-TfR scFvs are provided in Table 3 and include: (1) a polynucleotide encoding a HCVR that comprises the nucleotide sequence set forth in SEQ ID NO: 334, and a LCVR that comprises the nucleotide sequence set forth in SEQ ID NO: 339; (2) a polynucleotide encoding a HCVR that comprises the nucleotide sequence set forth in SEQ ID NO: 344, and a LCVR that comprises the nucleotide sequence set forth in SEQ ID NO: 349; (3) a polynucleotide encoding a HCVR that comprises the nucleotide sequence set forth in SEQ ID NO: 354, and a LCVR that comprises the nucleotide sequence set forth in SEQ ID NO: 359; (4) a polynucleotide encoding a HCVR that comprises the nucleotide sequence set forth in SEQ ID NO: 364, and a LCVR that comprises the nucleotide sequence set forth in SEQ ID NO: 369; (5) a polynucleotide encoding a HCVR that comprises the nucleotide sequence set forth in SEQ ID NO: 374, and a LCVR that comprises the nucleotide sequence set forth in SEQ ID NO: 379; (6) a polynucleotide encoding a HCVR that comprises the nucleotide sequence set forth in SEQ ID NO: 384, and a LCVR that comprises the nucleotide sequence set forth in SEQ ID NO: 389; (7) a polynucleotide encoding a HCVR that comprises the nucleotide sequence set forth in SEQ ID NO: 394, and a LCVR that comprises the nucleotide sequence set forth in SEQ ID NO: 399; (8) a polynucleotide encoding a HCVR that comprises the nucleotide sequence set forth in SEQ ID NO: 404, and a LCVR that comprises the nucleotide sequence set forth in SEQ ID NO: 409; (9) a polynucleotide encoding a HCVR that comprises the nucleotide sequence set forth in SEQ ID NO: 414, and a LCVR that comprises the nucleotide sequence set forth in SEQ ID NO: 419; (10) a polynucleotide encoding a HCVR that comprises the nucleotide sequence set forth in SEQ ID NO: 424, and a LCVR that comprises the nucleotide sequence set forth in SEQ ID NO: 429; (11) a polynucleotide encoding a HCVR that comprises the nucleotide sequence set forth in SEQ ID NO: 434, and a LCVR that comprises the nucleotide sequence set forth in SEQ ID NO: 439; (12) a polynucleotide encoding a HCVR that comprises the nucleotide sequence set forth in SEQ ID NO: 444, and a LCVR that comprises the nucleotide sequence set forth in SEQ ID NO: 449; (13) a polynucleotide encoding a HCVR that comprises the nucleotide sequence set forth in SEQ ID NO: 454, and a LCVR that comprises the nucleotide sequence set forth in SEQ ID NO: 459; (14) a polynucleotide encoding a HCVR that comprises the nucleotide sequence set forth in SEQ ID NO: 464, and a LCVR that comprises the nucleotide sequence set forth in SEQ ID NO: 469; (15) a polynucleotide encoding a HCVR that comprises the nucleotide sequence set forth in SEQ ID NO: 474, and a LCVR that comprises the nucleotide sequence set forth in SEQ ID NO: 479; (16) a polynucleotide encoding a HCVR that comprises the nucleotide sequence set forth in SEQ ID NO: 484, and a LCVR that comprises the nucleotide sequence set forth in SEQ ID NO: 489; (17) a polynucleotide encoding a HCVR that comprises the nucleotide sequence set forth in SEQ ID NO: 494, and a LCVR that comprises the nucleotide sequence set forth in SEQ ID NO: 499; (18) a polynucleotide encoding a HCVR that comprises the nucleotide sequence set forth in SEQ ID NO: 504, and a LCVR that comprises the nucleotide sequence set forth in SEQ ID NO: 509; (19) a polynucleotide encoding a HCVR that comprises the nucleotide sequence set forth in SEQ ID NO: 514, and a LCVR that comprises the nucleotide sequence set forth in SEQ ID NO: 519; (20) a polynucleotide encoding a HCVR that comprises the nucleotide sequence set forth in SEQ ID NO: 524, and a LCVR that comprises the nucleotide sequence set forth in SEQ ID NO: 529; (21) a polynucleotide encoding a HCVR that comprises the nucleotide sequence set forth in SEQ ID NO: 534, and a LCVR that comprises the nucleotide sequence set forth in SEQ ID NO: 539; (22) a polynucleotide encoding a HCVR that comprises the nucleotide sequence set forth in SEQ ID NO: 544, and a LCVR that comprises the nucleotide sequence set forth in SEQ ID NO: 549; (23) a polynucleotide encoding a HCVR that comprises the nucleotide sequence set forth in SEQ ID NO: 554, and a LCVR that comprises the nucleotide sequence set forth in SEQ ID NO: 559; (24) a polynucleotide encoding a HCVR that comprises the nucleotide sequence set forth in SEQ ID NO: 564, and a LCVR that comprises the nucleotide sequence set forth in SEQ ID NO: 569; (25) a polynucleotide encoding a HCVR that comprises the nucleotide sequence set forth in SEQ ID NO: 574, and a LCVR that comprises the nucleotide sequence set forth in SEQ ID NO: 579; (26) a polynucleotide encoding a HCVR that comprises the nucleotide sequence set forth in SEQ ID NO: 584, and a LCVR that comprises the nucleotide sequence set forth in SEQ ID NO: 589; (27) a polynucleotide encoding a HCVR that comprises the nucleotide sequence set forth in SEQ ID NO: 594, and a LCVR that comprises the nucleotide sequence set forth in SEQ ID NO: 599; (28) a polynucleotide encoding a HCVR that comprises the nucleotide sequence set forth in SEQ ID NO: 604, and a LCVR that comprises the nucleotide sequence set forth in SEQ ID NO: 609; (29) a polynucleotide encoding a HCVR that comprises the nucleotide sequence set forth in SEQ ID NO: 614, and a LCVR that comprises the nucleotide sequence set forth in SEQ ID NO: 619; (30) a polynucleotide encoding a HCVR that comprises the nucleotide sequence set forth in SEQ ID NO: 624, and a LCVR that comprises the nucleotide sequence set forth in SEQ ID NO: 629; (31) a polynucleotide encoding a HCVR that comprises the nucleotide sequence set forth in SEQ ID NO: 634, and a LCVR that comprises the nucleotide sequence set forth in SEQ ID NO: 639; or (32) a polynucleotide encoding a HCVR that comprises the nucleotide sequence set forth in SEQ ID NO: 644, and a LCVR that comprises the nucleotide sequence set forth in SEQ ID NO: 649, wherein the HCVR and LCVR are in either order.

In an embodiment, an anti-hTfR scFv, in V_{L}-(Gly₄Ser)₃-V_{H} format (Gly₄Ser=SEQ ID NO: 718), comprises the amino acid sequence set forth in any one of SEQ ID NOS: 656-687. Also contemplated are such fusions that are in the format V_{H}-(Gly₄Ser)₃-V_{L} (Gly₄Ser=SEQ ID NO: 718).

In another example, the TfR-binding delivery domain can be a Fab fragment (e.g., that binds specifically to human transferrin receptor). Fab fragments typically contain one complete light chain, VL and constant light domain, e.g., kappa (e.g., RTVAAPSVFIFPPSDEQLKSGTASVVCLLNNFYPREAKVQWKVDNALQSGNSQESVTEQ DSKDSTYSLS STLTLSKADYEKHKVYACEVTHQGLSSPVTKSFNRGEC (SEQ ID NO: 719)) and the V_{H} and IgG1 CH1 portion (e.g., ASTKGPSVFPLAPSSKSTSGGTAALGCLVKDYFPEPVTVSWNSGALTSGVHTFPAVLQSS GLYSLSSVV TVPSSSLGTQTYICNVNHKPSNTKVDKKVEPKSCDKTH (SEQ ID NO: 720)) or IgG4 CH1 (e.g., ASTKGPSVFPLAPCSRSTSESTAALGCLVKDYFPEPVTVSWNSGALTSGVHTFPAVLQSS GLYSLSSVVTVPSSSLGTKTYTCNVDHKPSNTKVDKRVESKYGPPLLQGSG (SEQ ID NO: 792)) of one heavy chain. Fab fragment antibodies can be generated by papain digestion of whole IgG antibodies to remove the entire Fc fragment, including the hinge region. In one example, the Fab protein can comprise: (1) a heavy chain variable region (HCVR) that comprises the amino acid sequence set forth in SEQ ID NO: 335, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 340, or LCDR1, LCDR2 and LCDR3 of such a LCVR-linked to the CL domain; (2) a heavy chain variable region (HCVR) that comprises the amino acid sequence set forth in SEQ ID NO: 345, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 350, or LCDR1, LCDR2 and LCDR3 of such a LCVR-linked to the CL domain; (3) a heavy chain variable region (HCVR) that comprises the amino acid sequence set forth in SEQ ID NO: 355, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 360, or LCDR1, LCDR2 and LCDR3 of such a LCVR-linked to the CL domain; (4) a heavy chain variable region (HCVR) that comprises the amino acid sequence set forth in SEQ ID NO: 365, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 370, or LCDR1, LCDR2 and LCDR3 of such a LCVR-linked to the CL domain; (5) a heavy chain variable region (HCVR) that comprises the amino acid sequence set forth in SEQ ID NO: 375, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 380, or LCDR1, LCDR2 and LCDR3 of such a LCVR-linked to the CL domain; (6) a heavy chain variable region (HCVR) that comprises the amino acid sequence set forth in SEQ ID NO: 385, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 390, or LCDR1, LCDR2 and LCDR3 of such a LCVR-linked to the CL domain; (7) a heavy chain variable region (HCVR) that comprises the amino acid sequence set forth in SEQ ID NO: 395, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 400, or LCDR1, LCDR2 and LCDR3 of such a LCVR-linked to the CL domain; (8) a heavy chain variable region (HCVR) that comprises the amino acid sequence set forth in SEQ ID NO: 405, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 410, or LCDR1, LCDR2 and LCDR3 of such a LCVR-linked to the CL domain; (9) a heavy chain variable region (HCVR) that comprises the amino acid sequence set forth in SEQ ID NO: 415, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 420, or LCDR1, LCDR2 and LCDR3 of such a LCVR-linked to the CL domain; (10) a heavy chain variable region (HCVR) that comprises the amino acid sequence set forth in SEQ ID NO: 425, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 430, or LCDR1, LCDR2 and LCDR3 of such a LCVR-linked to the CL domain; (11) a heavy chain variable region (HCVR) that comprises the amino acid sequence set forth in SEQ ID NO: 435, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 440, or LCDR1, LCDR2 and LCDR3 of such a LCVR-linked to the CL domain; (12) a heavy chain variable region (HCVR) that comprises the amino acid sequence set forth in SEQ ID NO: 445, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 450, or LCDR1, LCDR2 and LCDR3 of such a LCVR-linked to the CL domain; (13) a heavy chain variable region (HCVR) that comprises the amino acid sequence set forth in SEQ ID NO: 455, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 460, or LCDR1, LCDR2 and LCDR3 of such a LCVR-linked to the CL domain; (14) a heavy chain variable region (HCVR) that comprises the amino acid sequence set forth in SEQ ID NO: 465, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 470, or LCDR1, LCDR2 and LCDR3 of such a LCVR-linked to the CL domain; (15) a heavy chain variable region (HCVR) that comprises the amino acid sequence set forth in SEQ ID NO: 475, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 480, or LCDR1, LCDR2 and LCDR3 of such a LCVR-linked to the CL domain; (16) a heavy chain variable region (HCVR) that comprises the amino acid sequence set forth in SEQ ID NO: 485, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 490, or LCDR1, LCDR2 and LCDR3 of such a LCVR-linked to the CL domain; (17) a heavy chain variable region (HCVR) that comprises the amino acid sequence set forth in SEQ ID NO: 495, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 500, or LCDR1, LCDR2 and LCDR3 of such a LCVR-linked to the CL domain; (18) a heavy chain variable region (HCVR) that comprises the amino acid sequence set forth in SEQ ID NO: 505, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 510, or LCDR1, LCDR2 and LCDR3 of such a LCVR-linked to the CL domain; (19) a heavy chain variable region (HCVR) that comprises the amino acid sequence set forth in SEQ ID NO: 515, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 520, or LCDR1, LCDR2 and LCDR3 of such a LCVR-linked to the CL domain; (20) a heavy chain variable region (HCVR) that comprises the amino acid sequence set forth in SEQ ID NO: 525, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 530, or LCDR1, LCDR2 and LCDR3 of such a LCVR-linked to the CL domain; (21) a heavy chain variable region (HCVR) that comprises the amino acid sequence set forth in SEQ ID NO: 535, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 540, or LCDR1, LCDR2 and LCDR3 of such a LCVR-linked to the CL domain; (22) a heavy chain variable region (HCVR) that comprises the amino acid sequence set forth in SEQ ID NO: 545, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 550, or LCDR1, LCDR2 and LCDR3 of such a LCVR-linked to the CL domain; (23) a heavy chain variable region (HCVR) that comprises the amino acid sequence set forth in SEQ ID NO: 555, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 560, or LCDR1, LCDR2 and LCDR3 of such a LCVR-linked to the CL domain; (24) a heavy chain variable region (HCVR) that comprises the amino acid sequence set forth in SEQ ID NO: 565, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 570, or LCDR1, LCDR2 and LCDR3 of such a LCVR-linked to the CL domain; (25) a heavy chain variable region (HCVR) that comprises the amino acid sequence set forth in SEQ ID NO: 575, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 580, or LCDR1, LCDR2 and LCDR3 of such a LCVR-linked to the CL domain; (26) a heavy chain variable region (HCVR) that comprises the amino acid sequence set forth in SEQ ID NO: 585, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 590, or LCDR1, LCDR2 and LCDR3 of such a LCVR-linked to the CL domain; (27) a heavy chain variable region (HCVR) that comprises the amino acid sequence set forth in SEQ ID NO: 595, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 600, or LCDR1, LCDR2 and LCDR3 of such a LCVR-linked to the CL domain; (28) a heavy chain variable region (HCVR) that comprises the amino acid sequence set forth in SEQ ID NO: 605, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 610, or LCDR1, LCDR2 and LCDR3 of such a LCVR-linked to the CL domain; (29) a heavy chain variable region (HCVR) that comprises the amino acid sequence set forth in SEQ ID NO: 615, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 620, or LCDR1, LCDR2 and LCDR3 of such a LCVR-linked to the CL domain; (30) a heavy chain variable region (HCVR) that comprises the amino acid sequence set forth in SEQ ID NO: 625, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 630, or LCDR1, LCDR2 and LCDR3 of such a LCVR-linked to the CL domain; (31) a heavy chain variable region (HCVR) that comprises the amino acid sequence set forth in SEQ ID NO: 635, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 640, or LCDR1, LCDR2 and LCDR3 of such a LCVR-linked to the CL domain; and/or (32) a heavy chain variable region (HCVR) that comprises the amino acid sequence set forth in SEQ ID NO: 645, or a heavy chain variable region that includes HCDR1, HCDR2 and HCDR3 of such a HCVR-linked to the CH1 domain—and a light chain variable region (LCVR) that comprises the amino acid sequence set forth in SEQ ID NO: 650, or LCDR1, LCDR2 and LCDR3 of such a LCVR-linked to the CL domain. For example, the CH1 can be SEQ ID NO: 720 or 792.

In one example, the Fab protein can comprise: (1) a light chain that comprises the amino acid sequence set forth in SEQ ID NO: 721 and a heavy chain that comprises the amino acid sequence set forth in SEQ ID NO: 722 (31874B); (2) a light chain that comprises the amino acid sequence set forth in SEQ ID NO: 723 and a heavy chain that comprises the amino acid sequence set forth in SEQ ID NO: 724 (31863B); (3) a light chain that comprises the amino acid sequence set forth in SEQ ID NO: 725 and a heavy chain that comprises the amino acid sequence set forth in SEQ ID NO: 726 (69348); (4) a light chain that comprises the amino acid sequence set forth in SEQ ID NO: 727 and a heavy chain that comprises the amino acid sequence set forth in SEQ ID NO: 728 (69340); (5) a light chain that comprises the amino acid sequence set forth in SEQ ID NO: 729 and a heavy chain that comprises the amino acid sequence set forth in SEQ ID NO: 730 (69331); (6) a light chain that comprises the amino acid sequence set forth in SEQ ID NO: 731 and a heavy chain that comprises the amino acid sequence set forth in SEQ ID NO: 732 (69332); (7) a light chain that comprises the amino acid sequence set forth in SEQ ID NO: 733 and a heavy chain that comprises the amino acid sequence set forth in SEQ ID NO: 734 (69326); (8) a light chain that comprises the amino acid sequence set forth in SEQ ID NO: 735 and a heavy chain that comprises the amino acid sequence set forth in SEQ ID NO: 736 (69329); (9) a light chain that comprises the amino acid sequence set forth in SEQ ID NO: 737 and a heavy chain that comprises the amino acid sequence set forth in SEQ ID NO: 738 (69323); (10) a light chain that comprises the amino acid sequence set forth in SEQ ID NO: 739 and a heavy chain that comprises the amino acid sequence set forth in SEQ ID NO: 740 (69305); (11) a light chain that comprises the amino acid sequence set forth in SEQ ID NO: 741 and a heavy chain that comprises the amino acid sequence set forth in SEQ ID NO: 742 (69307); (12) a light chain that comprises the amino acid sequence set forth in SEQ ID NO: 743 and a heavy chain that comprises the amino acid sequence set forth in SEQ ID NO: 744 (12795B); (13) a light chain that comprises the amino acid sequence set forth in SEQ ID NO: 745 and a heavy chain that comprises the amino acid sequence set forth in SEQ ID NO: 746 or SEQ ID NO: 785 (12798B); (14) a light chain that comprises the amino acid sequence set forth in SEQ ID NO: 747 and a heavy chain that comprises the amino acid sequence set forth in SEQ ID NO: 748 or SEQ ID NO: 786 (12799B); (15) a light chain that comprises the amino acid sequence set forth in SEQ ID NO: 749 and a heavy chain that comprises the amino acid sequence set forth in SEQ ID NO: 750 (12801B); (16) a light chain that comprises the amino acid sequence set forth in SEQ ID NO: 751 and a heavy chain that comprises the amino acid sequence set forth in SEQ ID NO: 752 (12802B); (17) a light chain that comprises the amino acid sequence set forth in SEQ ID NO: 753 and a heavy chain that comprises the amino acid sequence set forth in SEQ ID NO: 754 (12808B); (18) a light chain that comprises the amino acid sequence set forth in SEQ ID NO: 755 and a heavy chain that comprises the amino acid sequence set forth in SEQ ID NO: 756 (12812B); (19) a light chain that comprises the amino acid sequence set forth in SEQ ID NO: 757 and a heavy chain that comprises the amino acid sequence set forth in SEQ ID NO: 758 (12816B); (20) a light chain that comprises the amino acid sequence set forth in SEQ ID NO: 759 and a heavy chain that comprises the amino acid sequence set forth in SEQ ID NO: 760 (12833B); (21) a light chain that comprises the amino acid sequence set forth in SEQ ID NO: 761 and a heavy chain that comprises the amino acid sequence set forth in SEQ ID NO: 762 (12834B); (22) a light chain that comprises the amino acid sequence set forth in SEQ ID NO: 763 and a heavy chain that comprises the amino acid sequence set forth in SEQ ID NO: 764 (12835B); (23) a light chain that comprises the amino acid sequence set forth in SEQ ID NO: 765 and a heavy chain that comprises the amino acid sequence set forth in SEQ ID NO: 766 or SEQ ID NO: 787 (12847B); (24) a light chain that comprises the amino acid sequence set forth in SEQ ID NO: 767 and a heavy chain that comprises the amino acid sequence set forth in SEQ ID NO: 768 (12848B); (25) a light chain that comprises the amino acid sequence set forth in SEQ ID NO: 769 and a heavy chain that comprises the amino acid sequence set forth in SEQ ID NO: 770 or SEQ ID NO: 788 (12843B); (26) a light chain that comprises the amino acid sequence set forth in SEQ ID NO: 771 and a heavy chain that comprises the amino acid sequence set forth in SEQ ID NO: 772 (12844B); (27) a light chain that comprises the amino acid sequence set forth in SEQ ID NO: 773 and a heavy chain that comprises the amino acid sequence set forth in SEQ ID NO: 774 or SEQ ID NO: 789 (12845B); (28) a light chain that comprises the amino acid sequence set forth in SEQ ID NO: 775 and a heavy chain that comprises the amino acid sequence set forth in SEQ ID NO: 776 or SEQ ID NO: 790 (12839B); (29) a light chain that comprises the amino acid sequence set forth in SEQ ID NO: 777 and a heavy chain that comprises the amino acid sequence set forth in SEQ ID NO: 778 (12841B); (30) a light chain that comprises the amino acid sequence set forth in SEQ ID NO: 779 and a heavy chain that comprises the amino acid sequence set forth in SEQ ID NO: 780 (12850B); (31) a light chain that comprises the amino acid sequence set forth in SEQ ID NO: 781 and a heavy chain that comprises the amino acid sequence set forth in SEQ ID NO: 782 (69261); or (32) a light chain that comprises the amino acid sequence set forth in SEQ ID NO: 783 and a heavy chain that comprises the amino acid sequence set forth in SEQ ID NO: 784 (69263).

[00553]“31874B”; “31863B”; “69348”; “69340”; “69331”; “69332”; “69326”; “69329”; “69323”; “69305”; “69307”; “12795B”; “12798B”; “12799B”; “12801B”; “12802B”; “12808B”; “12812B”; “12816B”; “12833B”; “12834B”; “12835B”; “12847B”; “12848B”; “12843B”; “12844B”; “12845B”; “12839B”; “12841B”; “12850B”; “69261”; and “69263” refer to anti-TfR:GAA fusion proteins, e.g., anti-TfR scFv:GAA or anti-TfR Fab:GAA, comprising a light chain variable region comprising the amino acid sequence set forth in SEQ ID NO: 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, or 650 (or a variant thereof), and a heavy chain variable region comprising the amino acid sequence set forth in SEQ ID NO: 335, 345, 355, 365, 375, 385, 395, 405, 415, 425, 435, 445, 455, 465, 475, 485, 495, 505, 515, 525, 535, 545, 555, 565, 575, 585, 595, 605, 615, 625, 635, or 645 (or a variant thereof); which, in the case of an scFv, can be fused together (in either order), e.g., by a peptide linker (e.g., (G₄S)₃) (G₄S=SEQ ID NO: 718), respectively; or that comprise a V_{H} that comprises the CDRs thereof (CDR-H1 (or a variant thereof), CDR-H2 (or a variant thereof) and CDR-H3 (or a variant thereof)) and/or a V_{L} that comprises the CDRs thereof (CDR-L1 (or a variant thereof), CDR-L2 (or a variant thereof) and CDR-L3 (or a variant thereof)), wherein the V_{H} fused to the V_{L} or the V_{L} fused to the V_{H}, in the case of an scFv, can be fused, e.g., by a peptide linker (e.g., (G₄S)₂) (G₄S=SEQ ID NO: 718), to GAA.

The TfR-binding delivery domain coding sequences in the constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a TfR-binding delivery domain coding sequence in a construct disclosed herein has one or more cryptic splice sites mutated or removed. In another example, a TfR-binding delivery domain coding sequence in a construct disclosed herein has all identified cryptic splice sites mutated or removed. In another example, a TfR-binding delivery domain coding sequence in a construct disclosed herein has one or more CpG dinucleotides removed (i.e., is CpG depleted). In another example, a TfR-binding delivery domain coding sequence in a construct disclosed herein has all CpG dinucleotides removed (i.e., is fully CpG depleted). In another example, a TfR-binding delivery domain coding sequence in a construct disclosed herein is codon optimized (e.g., codon optimized for expression in a human or mammal). In a specific example, a CDTfR63-binding delivery domain coding sequence in a construct disclosed herein has one or more CpG dinucleotides removed (i.e., is CpG depleted) and has one or more cryptic splice sites mutated or removed. In another specific example, a TfR-binding delivery domain coding sequence in a construct disclosed herein has all CpG dinucleotides removed and has one or more or all identified cryptic splice sites mutated or removed. In another specific example, a TfR-binding delivery domain coding sequence in a construct disclosed herein has one or more CpG dinucleotides removed (i.e., is CpG depleted) and is codon optimized (e.g., codon optimized for expression in a human or mammal). In another specific example, a TfR-binding delivery domain coding sequence in a construct disclosed herein has all CpG dinucleotides removed (i.e., is fully CpG depleted) and is codon optimized (e.g., codon optimized for expression in a human or mammal).

Various anti-TfR scFv coding sequences are provided. In one example, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 656-687 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 656-687 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 656-687 (and, e.g., retaining TfR-binding activity). Optionally, the anti-TfR scFv coding sequence in the above examples encodes an anti-TfR scFv protein comprising the sequence set forth in any one of SEQ ID NOS: 656-687. Optionally, the anti-TfR scFv coding sequence in the above examples encodes an anti-TfR scFv protein consisting essentially of the sequence set forth in any one of SEQ ID NOS: 656-687. Optionally, the anti-TfR scFv coding sequence in the above examples encodes an anti-TfR scFv protein consisting of the sequence set forth in any one of SEQ ID NOS: 656-687.

Various anti-TfR scFv coding sequences are provided. In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 705-717. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 705-717. In another example, the anti-TfR scFv coding sequence is (or comprises a sequence) at least 99%, at least 99.5%, or 100% identical to any one of SEQ ID NOS: 705-717. In another example, the anti-TfR scFv coding sequence comprises the sequence set forth in any one of SEQ ID NOS: 705-717. In another example, the anti-TfR scFv coding sequence consists essentially of the sequence set forth in any one of SEQ ID NOS: 705-717. In another example, the anti-TfR scFv coding sequence consists of the sequence set forth in any one of SEQ ID NOS: 705-717. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 658, 667, 669, and 672 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 658, 667, 669, and 672 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 658, 667, 669, and 672 (and, e.g., retaining TfR-binding activity). Optionally, the anti-TfR scFv coding sequence in the above examples encodes an anti-TfR scFv protein comprising the sequence set forth in any one of SEQ ID NOS: 658, 667, 669, and 672. Optionally, the anti-TfR scFv coding sequence in the above examples encodes an anti-TfR scFv protein consisting essentially of the sequence set forth in any one of SEQ ID NOS: 658, 667, 669, and 672. Optionally, the anti-TfR scFv coding sequence in the above examples encodes an anti-TfR scFv protein consisting of the sequence set forth in any one of SEQ ID NOS: 658, 667, 669, and 672.

Various anti-TfR scFv coding sequences are provided. In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 711-713 and 717. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 711-713 and 717. In another example, the anti-TfR scFv coding sequence is (or comprises a sequence) at least 99%, at least 99.5%, or 100% identical to any one of SEQ ID NOS: 711-713 and 717. In another example, the anti-TfR scFv coding sequence comprises the sequence set forth in any one of SEQ ID NOS: 711-713 and 717. In another example, the anti-TfR scFv coding sequence consists essentially of the sequence set forth in any one of SEQ ID NOS: 711-713 and 717. In another example, the anti-TfR scFv coding sequence consists of the sequence set forth in any one of SEQ ID NOS: 711-713 and 717. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein comprising the sequence set forth in SEQ ID NO: 672. Optionally, the anti-TfR scFv coding sequence in the above examples encodes an anti-TfR scFv protein consisting essentially of the sequence set forth in SEQ ID NO: 672. Optionally, the anti-TfR scFv coding sequence in the above examples encodes an anti-TfR scFv protein consisting of the sequence set forth in SEQ ID NO: 672.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 713. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 713 and encodes an anti-TfR scFv protein (or an anti-TfR scFv protein comprising a sequence) at least 99%, at least 99.5%, or 100% identical to SEQ ID NO: 672. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 713 and encodes an anti-TfR scFv protein comprising the sequence set forth in SEQ ID NO: 672. In another example, the anti-TfR scFv coding sequence is (or comprises a sequence) at least 95%, at least 96%, at least 97%, at least 98%, at least 99%, at least 99.5%, or 100% identical to SEQ ID NO: 713. In another example, the anti-TfR scFv coding sequence is (or comprises a sequence) at least 95%, at least 96%, at least 97%, at least 98%, at least 99%, at least 99.5%, or 100% identical to SEQ ID NO: 713 and encodes an anti-TfR scFv protein (or an anti-TfR scFv protein comprising a sequence) at least 99%, at least 99.5%, or 100% identical to SEQ ID NO: 672. In another example, the anti-TfR scFv coding sequence is (or comprises a sequence) at least 95%, at least 96%, at least 97%, at least 98%, at least 99%, at least 99.5%, or 100% identical to SEQ ID NO: 713 and encodes an anti-TfR scFv protein comprising the sequence set forth in SEQ ID NO: 672. In another example, the anti-TfR scFv coding sequence is (or comprises a sequence) at least 99%, at least 99.5%, or 100% identical to SEQ ID NO: 713. In another example, the anti-TfR scFv coding sequence is (or comprises a sequence) at least 99%, at least 99.5%, or 100% identical to SEQ ID NO: 713 and encodes an anti-TfR scFv protein (or an anti-TfR scFv protein comprising a sequence) at least 99%, at least 99.5%, or 100% identical to SEQ ID NO: 672. In another example, the anti-TfR scFv coding sequence is (or comprises a sequence) at least 99%, at least 99.5%, or 100% identical to SEQ ID NO: 713 and encodes an anti-TfR scFv protein comprising the sequence set forth in SEQ ID NO: 672. In another example, the anti-TfR scFv coding sequence comprises the sequence set forth in SEQ ID NO: 713. In another example, the anti-TfR scFv coding sequence consists essentially of the sequence set forth in SEQ ID NO: 713. In another example, the anti-TfR scFv coding sequence consists of the sequence set forth in SEQ ID NO: 713. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein comprising the sequence set forth in SEQ ID NO: 672. Optionally, the anti-TfR scFv coding sequence in the above examples encodes an anti-TfR scFv protein consisting essentially of the sequence set forth in SEQ ID NO: 672. Optionally, the anti-TfR scFv coding sequence in the above examples encodes an anti-TfR scFv protein consisting of the sequence set forth in SEQ ID NO: 672.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 705-713. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 705-713. In another example, the anti-TfR scFv coding sequence is (or comprises a sequence) at least 99%, at least 99.5%, or 100% identical to any one of SEQ ID NOS: 705-713. In another example, the anti-TfR scFv coding sequence comprises the sequence set forth in any one of SEQ ID NOS: 705-713. In another example, the anti-TfR scFv coding sequence consists essentially of the sequence set forth in any one of SEQ ID NOS: 705-713. In another example, the anti-TfR scFv coding sequence consists of the sequence set forth in any one of SEQ ID NOS: 705-713. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 658, 667, 669, and 672 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to any one of SEQ ID NOS: 658, 667, 669, and 672 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to any one of SEQ ID NOS: 658, 667, 669, and 672 (and, e.g., retaining TfR-binding activity). Optionally, the anti-TfR scFv coding sequence in the above examples encodes an anti-TfR scFv protein comprising the sequence set forth in any one of SEQ ID NOS: 658, 667, 669, and 672. Optionally, the anti-TfR scFv coding sequence in the above examples encodes an anti-TfR scFv protein consisting essentially of the sequence set forth in any one of SEQ ID NOS: 658, 667, 669, and 672. Optionally, the anti-TfR scFv coding sequence in the above examples encodes an anti-TfR scFv protein consisting of the sequence set forth in any one of SEQ ID NOS: 658, 667, 669, and 672.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 711-713. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 711-713. In another example, the anti-TfR scFv coding sequence is (or comprises a sequence) at least 99%, at least 99.5%, or 100% identical to any one of SEQ ID NOS: 711-713. In another example, the anti-TfR scFv coding sequence comprises the sequence set forth in any one of SEQ ID NOS: 711-713. In another example, the anti-TfR scFv coding sequence consists essentially of the sequence set forth in any one of SEQ ID NOS: 711-713. In another example, the anti-TfR scFv coding sequence consists of the sequence set forth in any one of SEQ ID NOS: 711-713. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein comprising the sequence set forth in SEQ ID NO: 672. Optionally, the anti-TfR scFv coding sequence in the above examples encodes an anti-TfR scFv protein consisting essentially of the sequence set forth in SEQ ID NO: 672. Optionally, the anti-TfR scFv coding sequence in the above examples encodes an anti-TfR scFv protein consisting of the sequence set forth in SEQ ID NO: 672.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 711. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 711 and encodes an anti-TfR scFv protein (or an anti-TfR scFv protein comprising a sequence) at least 99%, at least 99.5%, or 100% identical to SEQ ID NO: 672. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 711 and encodes an anti-TfR scFv protein comprising the sequence set forth in SEQ ID NO: 672. In another example, the anti-TfR scFv coding sequence is (or comprises a sequence) at least 95%, at least 96%, at least 97%, at least 98%, at least 99%, at least 99.5%, or 100% identical to SEQ ID NO: 711. In another example, the anti-TfR scFv coding sequence is (or comprises a sequence) at least 95%, at least 96%, at least 97%, at least 98%, at least 99%, at least 99.5%, or 100% identical to SEQ ID NO: 711 and encodes an anti-TfR scFv protein (or an anti-TfR scFv protein comprising a sequence) at least 99%, at least 99.5%, or 100% identical to SEQ ID NO: 672. In another example, the anti-TfR scFv coding sequence is (or comprises a sequence) at least 95%, at least 96%, at least 97%, at least 98%, at least 99%, at least 99.5%, or 100% identical to SEQ ID NO: 711 and encodes an anti-TfR scFv protein comprising the sequence set forth in SEQ ID NO: 672. In another example, the anti-TfR scFv coding sequence is (or comprises a sequence) at least 99%, at least 99.5%, or 100% identical to SEQ ID NO: 711. In another example, the anti-TfR scFv coding sequence is (or comprises a sequence) at least 99%, at least 99.5%, or 100% identical to SEQ ID NO: 711 and encodes an anti-TfR scFv protein (or an anti-TfR scFv protein comprising a sequence) at least 99%, at least 99.5%, or 100% identical to SEQ ID NO: 672. In another example, the anti-TfR scFv coding sequence is (or comprises a sequence) at least 99%, at least 99.5%, or 100% identical to SEQ ID NO: 711 and encodes an anti-TfR scFv protein comprising the sequence set forth in SEQ ID NO: 672. In another example, the anti-TfR scFv coding sequence comprises the sequence set forth in SEQ ID NO: 711. In another example, the anti-TfR scFv coding sequence consists essentially of the sequence set forth in SEQ ID NO: 711. In another example, the anti-TfR scFv coding sequence consists of the sequence set forth in SEQ ID NO: 711. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 672 (and, e.g., retaining TfR-binding activity). Optionally, the anti-TfR scFv coding sequence in the above examples encodes an anti-TfR scFv protein comprising the sequence set forth in SEQ ID NO: 672. Optionally, the anti-TfR scFv coding sequence in the above examples encodes an anti-TfR scFv protein consisting essentially of the sequence set forth in SEQ ID NO: 672. Optionally, the anti-TfR scFv coding sequence in the above examples encodes an anti-TfR scFv protein consisting of the sequence set forth in SEQ ID NO: 672.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 708-710. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 708-710. In another example, the anti-TfR scFv coding sequence is (or comprises a sequence) at least 99%, at least 99.5%, or 100% identical to any one of SEQ ID NOS: 708-710. In another example, the anti-TfR scFv coding sequence comprises the sequence set forth in any one of SEQ ID NOS: 708-710. In another example, the anti-TfR scFv coding sequence consists essentially of the sequence set forth in any one of SEQ ID NOS: 708-710. In another example, the anti-TfR scFv coding sequence consists of the sequence set forth in any one of SEQ ID NOS: 708-710. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 669 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 669 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 669 (and, e.g., retaining TfR-binding activity). Optionally, the anti-TfR scFv coding sequence in the above examples encodes an anti-TfR scFv protein comprising the sequence set forth in SEQ ID NO: 669. Optionally, the anti-TfR scFv coding sequence in the above examples encodes an anti-TfR scFv protein consisting essentially of the sequence set forth in SEQ ID NO: 669. Optionally, the anti-TfR scFv coding sequence in the above examples encodes an anti-TfR scFv protein consisting of the sequence set forth in SEQ ID NO: 669.

In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 708. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 708 and encodes an anti-TfR scFv protein (or an anti-TfR scFv protein comprising a sequence) at least 99%, at least 99.5%, or 100% identical to SEQ ID NO: 669. In another example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 708 and encodes an anti-TfR scFv protein comprising the sequence set forth in SEQ ID NO: 669. In another example, the anti-TfR scFv coding sequence is (or comprises a sequence) at least 95%, at least 96%, at least 97%, at least 98%, at least 99%, at least 99.5%, or 100% identical to SEQ ID NO: 708. In another example, the anti-TfR scFv coding sequence is (or comprises a sequence) at least 95%, at least 96%, at least 97%, at least 98%, at least 99%, at least 99.5%, or 100% identical to SEQ ID NO: 708 and encodes an anti-TfR scFv protein (or an anti-TfR scFv protein comprising a sequence) at least 99%, at least 99.5%, or 100% identical to SEQ ID NO: 669. In another example, the anti-TfR scFv coding sequence is (or comprises a sequence) at least 95%, at least 96%, at least 97%, at least 98%, at least 99%, at least 99.5%, or 100% identical to SEQ ID NO: 708 and encodes an anti-TfR scFv protein comprising the sequence set forth in SEQ ID NO: 669. In another example, the anti-TfR scFv coding sequence is (or comprises a sequence) at least 99%, at least 99.5%, or 100% identical to SEQ ID NO: 708. In another example, the anti-TfR scFv coding sequence is (or comprises a sequence) at least 99%, at least 99.5%, or 100% identical to SEQ ID NO: 708 and encodes an anti-TfR scFv protein (or an anti-TfR scFv protein comprising a sequence) at least 99%, at least 99.5%, or 100% identical to SEQ ID NO: 669. In another example, the anti-TfR scFv coding sequence is (or comprises a sequence) at least 99%, at least 99.5%, or 100% identical to SEQ ID NO: 708 and encodes an anti-TfR scFv protein comprising the sequence set forth in SEQ ID NO: 669. In another example, the anti-TfR scFv coding sequence comprises the sequence set forth in SEQ ID NO: 708. In another example, the anti-TfR scFv coding sequence consists essentially of the sequence set forth in SEQ ID NO: 708. In another example, the anti-TfR scFv coding sequence consists of the sequence set forth in SEQ ID NO: 708. The anti-TfR coding sequence can be, for example, CpG-depleted (e.g., fully CpG-depleted) and/or codon optimized. For example, the anti-TfR scFv coding sequence can be CpG depleted (e.g., fully CpG-depleted) and codon optimized. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 669 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 669 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 669 (and, e.g., retaining TfR-binding activity). Optionally, the anti-TfR scFv coding sequence in the above examples encodes an anti-TfR scFv protein comprising the sequence set forth in SEQ ID NO: 669. Optionally, the anti-TfR scFv coding sequence in the above examples encodes an anti-TfR scFv protein consisting essentially of the sequence set forth in SEQ ID NO: 669. Optionally, the anti-TfR scFv coding sequence in the above examples encodes an anti-TfR scFv protein consisting of the sequence set forth in SEQ ID NO: 669.

Various codon optimized anti-TfR scFv coding sequences are provided. The anti-TfR scFv coding sequence can be, for example, CpG-depleted (e.g., fully CpG depleted) and/or codon optimized (e.g., CpG depleted (e.g., fully CpG-depleted) and codon optimized). In one example, the anti-TfR scFv coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 705-707. In another example, the anti-TfR scFv coding sequence is (or comprises a sequence) at least 95%, at least 96%, at least 97%, at least 98%, at least 99%, at least 99.5%, or 100% identical to any one of SEQ ID NOS: 705-707. In another example, the anti-TfR scFv coding sequence is (or comprises a sequence) at least 99%, at least 99.5%, or 100% identical to any one of SEQ ID NOS: 705-707. In another example, the anti-TfR scFv coding sequence comprises the sequence set forth in any one of SEQ ID NOS: 705-707. In another example, the anti-TfR scFv coding sequence consists essentially of the sequence set forth in any one of SEQ ID NOS: 705-707. In another example, the anti-TfR scFv coding sequence consists of the sequence set forth in any one of SEQ ID NOS: 705-707. Optionally, the anti-TfR scFv coding sequence encodes an anti-TfR scFv protein (or an anti-TfR scFv protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 658 (and, e.g., retaining TfR-binding activity). Optionally, the anti-TfR scFv coding sequence encodes an anti-TfR scFv protein (or an anti-TfR scFv protein comprising a sequence) at least 95%, at least 96%, at least 97%, at least 98%, at least 99%, at least 99.5%, or 100% identical to SEQ ID NO: 658 (and, e.g., retaining TfR-binding activity). Optionally, the anti-TfR scFv coding sequence in the above examples encodes an anti-TfR scFv protein (or an anti-TfR scFv protein comprising a sequence) at least 99%, at least 99.5%, or 100% identical to SEQ ID NO: 658 (and, e.g., retaining TfR-binding activity). Optionally, the anti-TfR scFv coding sequence in the above examples encodes an anti-TfR scFv protein comprising the sequence set forth in SEQ ID NO: 658. Optionally, the anti-TfR scFv coding sequence in the above examples encodes an anti-TfR scFv protein consisting essentially of the sequence set forth in SEQ ID NO: 658. Optionally, the anti-TfR scFv coding sequence in the above examples encodes an anti-TfR scFv protein consisting of the sequence set forth in SEQ ID NO: 658.

When specific anti-TfR scFv or multidomain therapeutic protein nucleic acid constructs sequences are disclosed herein, they are meant to encompass the sequence disclosed or the reverse complement of the sequence. For example, if an anti-TfR scFv or multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the anti-TfR scFv or multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (5) Bidirectional Constructs

The nucleic acid constructs disclosed herein can be bidirectional constructs. Such bidirectional constructs can allow for enhanced insertion and expression of encoded polypeptide of interest. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus or a cleavage site or target insertion site, allowing the expression of the polypeptide of interest when inserted in either orientation, thereby enhancing expression efficiency.

A bidirectional construct as disclosed herein can comprise at least two nucleic acid segments, wherein a first segment comprises a first coding sequence for the polypeptide of interest, and a second segment comprises the reverse complement of a second coding sequence for the polypeptide of interest, or vice versa. However, other bidirectional constructs disclosed herein can comprise at least two nucleic acid segments, wherein the first segment comprises a coding sequence for a polypeptide of interest, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the polypeptide of interest or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode a polypeptide of interest, the at least two segments can encode the same polypeptide of interest or different polypeptides of interest. The different polypeptides of interest can be at least about 90%, at least about 95%, at least about 96%, at least about 97%, at least about 98%, at least about 99%, or at least about 99.5% identical. For example, the first segment can encode a wild type polypeptide of interest or fragment thereof, and the second segment can encode a variant of the polypeptide of interest or fragment thereof, or vice versa. Alternatively, the first segment can encode a first variant polypeptide of interest, and the second segment can encode a second variant polypeptide of interest that is different from the first variant polypeptide of interest. Preferably, the two segments encode the same polypeptide of interest (i.e., 100% identical).

Even when the two segments encode the same polypeptide of interest, the coding sequence for the polypeptide of interest in the first segment can differ from the coding sequence for the polypeptide of interest in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second polypeptide of interest coding sequence can be codon optimized or may use one or more alternative codons for one or more amino acids of the same polypeptide of interest (i.e., same amino acid sequence) encoded by the polypeptide of interest coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a polypeptide of interest coding sequence that adopts different codon usage from that of the polypeptide of interest coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of the polypeptide of interest.

In some cases, the bidirectional nucleic acid construct does not comprise a promoter that drives the expression of the polypeptide of interest. For example, the expression of the polypeptide of interest can be driven by a promoter of the host cell (e.g., the endogenous ALB promoter when the transgene is integrated into a host cell's ALB locus). In other cases, the bidirectional nucleic acid construct can comprise one or more promoters operably linked to the coding sequences for the polypeptide of interest. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first polypeptide of interest coding sequence and/or the reverse complement of a promoter that drives expression of the reverse complement of the second polypeptide of interest coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a polypeptide of interest.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a BGH polyadenylation signal. In a specific example, one polyadenylation signal can comprise, consist essentially of, or consist of SEQ ID NO: 284, and the other polyadenylation signal can comprise, consist essentially of, or consist of SEQ ID NO: 285.

In one example, either polyadenylation signal can comprise a BGH polyadenylation signal. For example, the BGH polyadenylation signal can comprise, consist essentially of, or consist of SEQ ID NO: 858. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 859. In another example, a synthetic polyadenylation signal can be used. For example, the synthetic polyadenylation signal can comprise, consist essentially of, or consist of SEQ ID NO: 860. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 858, and the unidirectional SV40 late polyadenylation signal can comprise, consist essentially of, or consist of SEQ ID NO: 859. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 902. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 858, and the synthetic polyadenylation signal can comprise, consist essentially of, or consist of SEQ ID NO: 860.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 859. In some embodiments, the unidirectional SV40 late polyadenylation signal comprises, consists essentially of, or consists of the sequence set forth in SEQ ID NO: 859.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 858. In some embodiments, the BGH polyadenylation signal comprises, consists essentially of, or consists of the sequence set forth in SEQ ID NO: 858. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 902. In some embodiments, the combination of the BGH polyadenylation signal and the unidirectional SV40 late polyadenylation signal comprises, consists essentially of, or consists of the sequence set forth in SEQ ID NO: 902.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the polypeptide of interest coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GAL7, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. In a specific example, both splice acceptors can comprise, consist essentially of, or consist of SEQ ID NO: 286.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from a gene encoding the polypeptide of interest. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary bidirectional construct, the second segment is located 3′ of the first segment, the first polypeptide of interest coding sequence and the second polypeptide of interest coding sequence both encode the same human polypeptide of interest, the second polypeptide of interest coding sequence adopts a different codon usage from the codon usage of the first polypeptide of interest coding sequence, the first segment comprises a first polyadenylation signal sequence located 3′ of the first polypeptide of interest coding sequence, the second segment comprises a reverse complement of a second polyadenylation signal sequence located 5′ of the reverse complement of the second polypeptide of interest coding sequence, the first segment comprises a first splice acceptor site located 5′ of the first polypeptide of interest coding sequence, the second segment comprises a reverse complement of a second splice acceptor site located 3′ of the reverse complement of the second polypeptide of interest coding sequence, the nucleic acid construct does not comprise a promoter that drives expression of the first polypeptide of interest or the second polypeptide of interest, and optionally the nucleic acid construct does not comprise a homology arm.

### (6) Unidirectional Constructs

The nucleic acid constructs disclosed herein can be unidirectional constructs. When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the coding sequence for the polypeptide of interest can be codon-optimized for expression in a host cell. For example, the coding sequence can be codon optimized or may use one or more alternative codons for one or more amino acids of the polypeptide of interest (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of polypeptide of interest. For example, the expression of the polypeptide of interest can be driven by a promoter of the host cell (e.g., the endogenous ALB promoter when the transgene is integrated into a host cell's ALB locus). In other cases, the unidirectional nucleic acid construct can comprise one or more promoters operably linked to the coding sequence for the polypeptide of interest. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the coding sequence for the polypeptide of interest.

The unidirectional constructs can, in some cases, comprise one or more polyadenylation tail sequences or polyadenylation signal sequences. Some unidirectional constructs can comprise a polyadenylation signal sequence located 3′ of the coding sequence for the polypeptide of interest. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a BGH polyadenylation signal. For example, the polyadenylation signal can be an SV40 polyadenylation signal or a BGH polyadenylation signal. In a specific example, the polyadenylation signal can comprise, consist essentially of, or consist of SEQ ID NO: 284. In another specific example, the polyadenylation signal can comprise, consist essentially of, or consist of SEQ ID NO: 285.

In one example, the polyadenylation signal can comprise a BGH polyadenylation signal. For example, the BGH polyadenylation signal can comprise, consist essentially of, or consist of SEQ ID NO: 858. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 859. In another example, a synthetic polyadenylation signal can be used. For example, the synthetic polyadenylation signal can comprise, consist essentially of, or consist of SEQ ID NO: 860. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 858, and the unidirectional SV40 late polyadenylation signal can comprise, consist essentially of, or consist of SEQ ID NO: 859. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 902. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 858, and the synthetic polyadenylation signal can comprise, consist essentially of, or consist of SEQ ID NO: 860.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 859. In some embodiments, the unidirectional SV40 late polyadenylation signal comprises, consists essentially of, or consists of the sequence set forth in SEQ ID NO: 859.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 858. In some embodiments, the BGH polyadenylation signal comprises, consists essentially of, or consists of the sequence set forth in SEQ ID NO: 858. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 902. In some embodiments, the combination of the BGH polyadenylation signal and the unidirectional SV40 late polyadenylation signal comprises, consists essentially of, or consists of the sequence set forth in SEQ ID NO: 902.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the coding sequence for the polypeptide of interest (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the coding sequence for the polypeptide of interest. In a specific example, the splice acceptor is a mouse Alb exon 2 splice acceptor. In a specific example, the splice acceptor can comprise, consist essentially of, or consist of SEQ ID NO: 286.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a splice acceptor from the gene encoding the polypeptide of interest. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

In an exemplary unidirectional construct, the construct comprises a polyadenylation signal sequence located 3′ of the coding sequence for the polypeptide of interest, the construct comprises a splice acceptor site located 5′ of the coding sequence for the polypeptide of interest, and the nucleic acid construct does not comprise a promoter that drives expression of the polypeptide of interest, and optionally the nucleic acid construct does not comprise a homology arm.

### (7) F9 Nucleic Acid Constructs

The F9 nucleic acid constructs disclosed herein can be bidirectional constructs. Such bidirectional constructs can allow for enhanced insertion and expression of encoded FIX. When used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a target genomic locus, allowing the expression of FIX when inserted in either orientation, thereby enhancing expression efficiency, as exemplified herein. For example, when used in combination with a nuclease agent (e.g., CRISPR/Cas system, zinc finger nuclease (ZFN) system; transcription activator-like effector nuclease (TALEN) system) as described herein, the bidirectionality of the nucleic acid construct allows the construct to be inserted in either direction (i.e., is not limited to insertion in one direction) within a cleavage site or target insertion site, allowing the expression of FIX when inserted in either orientation, thereby enhancing insertion and expression efficiency, as exemplified herein.

A bidirectional construct as disclosed herein can comprise at least two nucleic acid segments, wherein a first segment comprises a first FIX coding sequence, and a second segment comprises the reverse complement of a second FIX coding sequence, or vice versa. However, other bidirectional constructs disclosed herein can comprise at least two nucleic acid segments, wherein the first segment comprises a FIX coding sequence, and the second segment comprises the reverse complement of a coding sequence for another protein, or vice versa. A reverse complement refers to a sequence that is a complement sequence of a reference sequence, wherein the complement sequence is written in the reverse orientation. For example, for a hypothetical sequence 5′-CTGGACCGA-3′, the perfect complement sequence is 3′-GACCTGGCT-5′, and the perfect reverse complement is written 5′-TCGGTCCAG-3′. A reverse complement sequence need not be perfect and may still encode the same polypeptide or a similar polypeptide as the reference sequence. Due to codon usage redundancy, a reverse complement can diverge from a reference sequence that encodes the same polypeptide. The coding sequences can optionally comprise one or more additional sequences, such as sequences encoding amino- or carboxy-terminal amino acid sequences such as a signal sequence, label sequence (e.g., HiBit), or heterologous functional sequence (e.g., nuclear localization sequence (NLS) or self-cleaving peptide) linked to the FIX or other protein.

When specific bidirectional construct sequences are disclosed herein, they are meant to encompass the sequence disclosed or the reverse complement of the sequence. For example, if a bidirectional construct disclosed herein consists of the hypothetical sequence 5′-CTGGACCGA-3′, it is also meant to encompass the reverse complement of that sequence (5′-TCGGTCCAG-3′). Likewise, when bidirectional construct elements are disclosed herein in a specific 5′ to 3′ order, they are also meant to encompass the reverse complement of the order of those elements. For example, if a bidirectional construct is disclosed herein that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. One reason for this is that, in many embodiments disclosed herein, the bidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

When the at least two segments both encode FIX, the at least two segments can encode the same FIX protein or different FIX proteins. The different FIX proteins can be at least about 90%, at least about 95%, at least about 96%, at least about 97%, at least about 98%, at least about 99%, or at least about 99.5% identical. For example, the first segment can encode a wild type FIX protein or fragment thereof, and the second segment can encode a variant FIX protein or fragment thereof, or vice versa. Alternatively, the first segment can encode a first variant FIX protein, and the second segment can encode a second variant FIX protein that is different from the first variant FIX protein. Preferably, the two segments encode the same FIX protein (i.e., 100% identical).

Even when the two segments encode the same FIX protein, the FIX coding sequence in the first segment can differ from the FIX coding sequence in the second segment. In some bidirectional constructs, the codon usage in the first coding sequence is the same as the codon usage in the second coding sequence. In other bidirectional constructs, the second coding sequence adopts a different codon usage from the codon usage of the first coding sequence in order to reduce hairpin formation. One or both of the coding sequences can be codon-optimized for expression in a host cell. In some bidirectional constructs, only one of the coding sequences is codon-optimized. In some bidirectional constructs, the first coding sequence is codon-optimized. In some bidirectional constructs, the second coding sequence is codon-optimized. In some bidirectional constructs, both coding sequences are codon-optimized. For example, the second FIX coding sequence can be codon optimized or may use one or more alternative codons for one or more amino acids of the same FIX (i.e., same amino acid sequence) encoded by the FIX coding sequence in the first segment. An alternative codon as used herein refers to variations in codon usage for a given amino acid, and may or may not be a preferred or optimized codon (codon optimized) for a given expression system. Preferred codon usage, or codons that are well-tolerated in a given system of expression are known.

In one example, the second segment comprises a reverse complement of a FIX coding sequence that adopts different codon usage from that of the FIX coding sequence in the first segment in order to reduce hairpin formation. Such a reverse complement forms base pairs with fewer than all nucleotides of the coding sequence in the first segment, yet it optionally encodes the same polypeptide. In one example, the reverse complement sequence in the second segment is not substantially complementary (e.g., not more than 70% complementary) to the coding sequence in the first segment. In other cases, however, the second segment comprises a reverse complement sequence that is highly complementary (e.g., at least 90% complementary) to the coding sequence in the first segment.

The second segment can have any percentage of complementarity to the first segment. For example, the second segment sequence can have at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 97%, or at least about 99% complementarity to the first segment. As another example, the second segment sequence can have less than about 30%, less than about 35%, less than about 40%, less than about 45%, less than about 50%, less than about 55%, less than about 60%, less than about 65%, less than about 70%, less than about 75%, less than about 80%, less than about 85%, less than about 90%, less than about 95%, less than about 97%, or less than about 99% complementarity to the first segment. The reverse complement of the second coding sequence can be, in some nucleic acid constructs, not substantially complementary (e.g., not more than 70% complementary) to the first coding sequence, not substantially complementary to a fragment of the first coding sequence, highly complementary (e.g., at least 90% complementary) to the first coding sequence, highly complementary to a fragment of the first coding sequence, about 50% to about 80% identical to the reverse complement of the first coding sequence, or about 60% to about 100% identical to the reverse complement of the first coding sequence.

The bidirectional constructs disclosed herein can be modified to include any suitable structural feature as needed for any particular use and/or that confers one or more desired function. For example, the bidirectional nucleic acid constructs disclosed herein need not comprise a homology arm and/or can be, for example, homology-independent donor constructs. Owing in part to the bidirectional function of the nucleic acid constructs, the bidirectional constructs can be inserted into a genomic locus in either direction as described herein to allow for efficient insertion and/or expression of FIX.

In some cases, the bidirectional nucleic acid construct does not comprise a promoter that drives the expression of FIX. For example, the expression of FIX can be driven by a promoter of the host cell (e.g., the endogenous ALB promoter when the transgene is integrated into a host cell's ALB locus). In other cases, the bidirectional nucleic acid construct can comprise one or more promoters operably linked to the FIX coding sequences. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some bidirectional constructs can comprise a promoter that drives expression of the first FIX coding sequence and/or the reverse complement of a promoter that drives expression of the reverse complement of the second FIX coding sequence.

The bidirectional constructs disclosed herein can be modified to include or exclude any suitable structural feature as needed for any particular use and/or that confers one or more desired functions. For example, some bidirectional nucleic acid constructs disclosed herein do not comprise a homology arm. Owing in part to the bidirectional function of the nucleic acid construct, the bidirectional construct can be inserted into a genomic locus in either direction (orientation) as described herein to allow for efficient insertion and/or expression of a heterologous FIX.

The bidirectional constructs can, in some cases, comprise one or more (e.g., two) polyadenylation tail sequences or polyadenylation signal sequences. In some bidirectional constructs, the first segment can comprise a polyadenylation signal sequence. In some bidirectional constructs, the second segment can comprise a polyadenylation signal sequence. In some bidirectional constructs, the first segment can comprise a first polyadenylation signal sequence, and the second segment can comprise a second polyadenylation signal sequence (e.g., a reverse complement of a polyadenylation signal sequence). In some bidirectional constructs, the first segment can comprise a first polyadenylation signal sequence located 3′ of the first coding sequence. In some bidirectional constructs, the second segment can comprise a reverse complement of a second polyadenylation signal sequence located 5′ of the reverse complement of the second coding sequence. In some bidirectional constructs, the first segment can comprise a first polyadenylation signal sequence located 3′ of the first coding sequence, and the second segment can comprise a reverse complement of a second polyadenylation signal sequence located 5′ of the reverse complement of the second coding sequence. The first and second polyadenylation signal sequences can be the same or different. In one example, the first and second polyadenylation signals are different. In a specific example, the first polyadenylation signal is a simian virus 40 (SV40) late polyadenylation signal (or a variant thereof), and the second polyadenylation signal is a bovine growth hormone (BGH) polyadenylation signal (or a variant thereof), or vice versa. For example, one polyadenylation signal can be an SV40 polyadenylation signal, and the other polyadenylation signal can be a CpG-depleted BGH polyadenylation signal. In a specific example, one polyadenylation signal can comprise, consist essentially of, or consist of SEQ ID NO: 98, and the other polyadenylation signal can comprise, consist essentially of, or consist of SEQ ID NO: 99.

In some bidirectional constructs, both the first segment and the second segment comprise a polyadenylation tail sequence. Methods of designing a suitable polyadenylation tail sequence are known. For example, in some bidirectional constructs, one or both of the first and second segment comprises a polyadenylation tail sequence and/or a polyadenylation signal sequence downstream of an open reading frame (i.e., a polyadenylation tail sequence and/or a polyadenylation signal sequence 3′ of a coding sequence, or a reverse complement of a polyadenylation tail sequence and/or a polyadenylation signal sequence 5′ of a reverse complement of a coding sequence). The polyadenylation tail sequence can be encoded, for example, as a “poly-A” stretch downstream of the FIX coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes. In some bidirectional constructs, a single bidirectional terminator can be used to terminate RNA polymerase transcription in either the sense or the antisense direction (i.e., to terminate RNA polymerase transcription from both the first segment and the second segment). Examples of bidirectional terminators include the ARO4, TRP1, TRP4, ADH1, CYC1, GAL1, GAL7, and GAL10 terminators.

The bidirectional constructs can, in some cases, comprise one or more (e.g., two) splice acceptor sites. In some bidirectional constructs, the first segment can comprise a splice acceptor site. In some bidirectional constructs, the second segment can comprise a splice acceptor site. In some bidirectional constructs, the first segment can comprise a first splice acceptor site, and the second segment can comprise a second splice acceptor site (e.g., a reverse complement of a splice acceptor site). In some bidirectional constructs, the first segment comprises a first splice acceptor site located 5′ of the first coding sequence. In some bidirectional constructs, the second segment comprises a reverse complement of a second splice acceptor site located 3′ of the reverse complement of the second coding sequence. In some bidirectional constructs, the first segment comprises a first splice acceptor site located 5′ of the first coding sequence, and the second segment comprises a reverse complement of a second splice acceptor site located 3′ of the reverse complement of the second coding sequence. The first and second splice acceptor sites can be the same or different. In a specific example, both splice acceptors are mouse Alb exon 2 splice acceptors. In a specific example, both splice acceptors can comprise, consist essentially of, or consist of SEQ ID NO: 100.

A bidirectional construct may comprise a first coding sequence that encodes a first coding sequence linked to a splice acceptor and a reverse complement of a second coding sequence operably linked to the reverse complement of a splice acceptor. The bidirectional constructs disclosed herein can also comprise a splice acceptor site on either or both ends of the construct, or splice acceptor sites in both the first segment and the second segment (e.g., a splice acceptor site 5′ of a coding sequence, or a reverse complement of a splice acceptor 3′ of a reverse complement of a coding sequence). The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a F9 splice acceptor (e.g., the F9 splice acceptor used in the splicing together of exons 1 and 2 of F9). For example, such a splice acceptor can be derived from the human F9 gene. Alternatively, such a splice acceptor can be derived from the mouse F9 gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes. The splice acceptors used in a bidirectional construct may be the same or different. In a specific example, both splice acceptors are mouse Alb exon 2 splice acceptors.

The bidirectional constructs can be circular or linear. For example, a bidirectional construct can be linear. The first and second segments can be joined in a linear manner through a linker sequence. For example, the 5′ end of the second segment that comprises a reverse complement sequence can be linked to the 3′ end of the first segment. Alternatively, the 5′ end of the first segment can be linked to the 3′ end of the second segment that comprises a reverse complement sequence. The linker can be any suitable length. For example, the linker can be between about 5 to about 2000 nucleotides in length. As an example, the linker sequence can be about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 80, about 85, about 90, about 95, about 100, about 150, about 200, about 250, about 300, about 500, about 1000, about 1500, about 2000, or more nucleotides in length. Other structural elements in addition to, or instead of, a linker sequence, can also be inserted between the first and second segments.

The bidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the bidirectional construct is single-stranded (e.g., single-stranded DNA).

The bidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the bidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The FIX coding sequences in the bidirectional constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a bidirectional construct disclosed herein has one or more cryptic splice sites mutated or removed. In another example, a FIX coding sequence in a bidirectional construct disclosed herein has all identified cryptic splice sites mutated or removed. In another example, a FIX coding sequence in a bidirectional construct disclosed herein has one or more CpG dinucleotides removed (i.e., is CpG depleted). In another example, a FIX coding sequence in a bidirectional construct disclosed herein has all but one CpG dinucleotides removed. In another example, a FIX coding sequence in a bidirectional construct disclosed herein has all CpG dinucleotides removed (i.e., is fully CpG depleted). In another example, a FIX coding sequence in a bidirectional construct disclosed herein is codon optimized (e.g., codon optimized for expression in a human or mammal). In a specific example, a FIX coding sequence in a bidirectional construct disclosed herein has one or more CpG dinucleotides removed (i.e., is CpG depleted) and has one or more cryptic splice sites mutated or removed. In another specific example, a FIX coding sequence in a bidirectional construct disclosed herein has all but one CpG dinucleotides removed and has one or more or all identified cryptic splice sites mutated or removed. In another specific example, a FIX coding sequence in a bidirectional construct disclosed herein has one or more CpG dinucleotides removed (i.e., is CpG depleted) and is codon optimized (e.g., codon optimized for expression in a human or mammal). In another specific example, a FIX coding sequence in a bidirectional construct disclosed herein has all CpG dinucleotides removed (i.e., is fully CpG depleted) and is codon optimized (e.g., codon optimized for expression in a human or mammal).

In one specific example, one FIX coding sequence in a bidirectional construct disclosed herein has one or more CpG dinucleotides removed (i.e., is CpG depleted) and has one or more cryptic splice sites mutated or removed, and the other FIX coding sequence in the bidirectional construct disclosed herein has one or more CpG dinucleotides removed (i.e., is CpG depleted) and is codon optimized (e.g., codon optimized for expression in a human or mammal). In another specific example, one FIX coding sequence in a bidirectional construct disclosed herein has all but one CpG dinucleotides removed and has one or more or all identified cryptic splice sites mutated or removed, and the other FIX coding sequence in the bidirectional construct disclosed herein has all CpG dinucleotides removed (i.e., is fully CpG depleted) and is codon optimized (e.g., codon optimized for expression in a human or mammal).

In an exemplary bidirectional construct, the second segment is located 3′ of the first segment, the first FIX coding sequence and the second FIX coding sequence both encode the same human FIX protein, the second FIX coding sequence adopts a different codon usage from the codon usage of the first FIX coding sequence, the first segment comprises a first polyadenylation signal sequence located 3′ of the first FIX coding sequence, the second segment comprises a reverse complement of a second polyadenylation signal sequence located 5′ of the reverse complement of the second FIX coding sequence, the first segment comprises a first splice acceptor site located 5′ of the first FIX coding sequence, the second segment comprises a reverse complement of a second splice acceptor site located 3′ of the reverse complement of the second FIX coding sequence, the nucleic acid construct does not comprise a promoter that drives expression of the first FIX protein or the second FIX protein, and optionally the nucleic acid construct does not comprise a homology arm.

In one example of a bidirectional construct, the first FIX protein coding sequence and the second FIX protein coding sequence are different but encode the same FIX protein sequence, and one of the FIX coding sequences is CpG-depleted (e.g., fully CpG-depleted) and/or codon optimized (e.g., CpG-depleted and codon optimized or fully CpG-depleted and codon optimized). In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 64-73. See, e.g., WO 2023/077012 and US 2023-0149563, each of which is herein incorporated by reference in its entirety for all purposes. In another example, the one of the FIX coding sequences is (or comprises a sequence) at least 95%, at least 96%, at least 97%, at least 98%, at least 99%, at least 99.5%, or 100% identical to any one of SEQ ID NOS: 64-73. In another example, the one of the FIX coding sequences is (or comprises a sequence) at least 99%, at least 99.5%, or 100% identical to any one of SEQ ID NOS: 64-73. In another example, the one of the FIX coding sequences comprises the sequence set forth in any one of SEQ ID NOS: 64-73. In another example, the one of the FIX coding sequences consists essentially of the sequence set forth in any one of SEQ ID NOS: 64-73. In another example, the one of the FIX coding sequences consists of the sequence set forth in any one of SEQ ID NOS: 64-73. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 97 (and, e.g., retaining the activity of native FIX). Optionally, the one of the FIX coding sequences in the above examples encodes a FIX protein comprising the sequence set forth in SEQ ID NO: 97. Optionally, the one of the FIX coding sequences in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 66-73. In another example, the one of the FIX coding sequences is (or comprises a sequence) at least 95%, at least 96%, at least 97%, at least 98%, at least 99%, at least 99.5%, or 100% identical to any one of SEQ ID NOS: 66-73. In another example, the one of the FIX coding sequences is (or comprises a sequence) at least 99%, at least 99.5%, or 100% identical to any one of SEQ ID NOS: 66-73. In another example, the one of the FIX coding sequences comprises the sequence set forth in any one of SEQ ID NOS: 66-73. In another example, the one of the FIX coding sequences consists essentially of the sequence set forth in any one of SEQ ID NOS: 66-73. In another example, the one of the FIX coding sequences consists of the sequence set forth in any one of SEQ ID NOS: 66-73. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) comprising the sequence set forth in SEQ ID NO: 97. Optionally, the one of the FIX coding sequences in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 or 67. In another example, the one of the FIX coding sequences is (or comprises a sequence) at least 95%, at least 96%, at least 97%, at least 98%, at least 99%, at least 99.5%, or 100% identical to SEQ ID NO: 68 or 67. In another example, the one of the FIX coding sequences is (or comprises a sequence) at least 99%, at least 99.5%, or 100% identical to SEQ ID NO: 68 or 67. In another example, the one of the FIX coding sequences comprises the sequence set forth in SEQ ID NO: 68 or 67. In another example, the one of the FIX coding sequences consists essentially of the sequence set forth in SEQ ID NO: 68 or 67. In another example, the one of the FIX coding sequences consists of the sequence set forth in SEQ ID NO: 68 or 67. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 97 (and, e.g., retaining the activity of native FIX). Optionally, the one of the FIX coding sequences in the above examples encodes a FIX protein comprising the sequence set forth in SEQ ID NO: 97. Optionally, the one of the FIX coding sequences in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. In another example, the one of the FIX coding sequences is (or comprises a sequence) at least 95%, at least 96%, at least 97%, at least 98%, at least 99%, at least 99.5%, or 100% identical to SEQ ID NO: 68. In another example, the one of the FIX coding sequences is (or comprises a sequence) at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the one of the FIX coding sequences is (or comprises a sequence) at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. In another example, the one of the FIX coding sequences is (or comprises a sequence) at least 99%, at least 99.5%, or 100% identical to SEQ ID NO: 68. In another example, the one of the FIX coding sequences is (or comprises a sequence) at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the one of the FIX coding sequences is (or comprises a sequence) at least 99%, at least 99.5%, or 100% identical to SEQ ID NO: 68 and encodes a FIX protein comprising the sequence set forth in SEQ ID NO: 97. In another example, the one of the FIX coding sequences comprises the sequence set forth in SEQ ID NO: 68. In another example, the one of the FIX coding sequences consists essentially of the sequence set forth in SEQ ID NO: 68. In another example, the one of the FIX coding sequences consists of the sequence set forth in SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 97 (and, e.g., retaining the activity of native FIX). Optionally, the one of the FIX coding sequences in the above examples encodes a FIX protein comprising the sequence set forth in SEQ ID NO: 97. Optionally, the one of the FIX coding sequences in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

Various native and optimized native FIX coding sequences are also provided. In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 60-63. In another example, the one of the FIX coding sequences is (or comprises a sequence) at least 95%, at least 96%, at least 97%, at least 98%, at least 99%, at least 99.5%, or 100% identical to any one of SEQ ID NOS: 60-63. In another example, the one of the FIX coding sequences is (or comprises a sequence) at least 99%, at least 99.5%, or 100% identical to any one of SEQ ID NOS: 60-63. In another example, the one of the FIX coding sequences comprises the sequence set forth in any one of SEQ ID NOS: 60-63. In another example, the one of the FIX coding sequences consists essentially of the sequence set forth in any one of SEQ ID NOS: 60-63. In another example, the one of the FIX coding sequences consists of the sequence set forth in any one of SEQ ID NOS: 60-63. The one of the FIX coding sequences can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the one of the FIX coding sequences can be CpG depleted (e.g., all but one CpG dinucleotides removed or fully CpG-depleted) and modified to mutate one or more cryptic splice donor sequences (e.g., all identified cryptic splice donor sequences). Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 97 (and, e.g., retaining the activity of native FIX). Optionally, the one of the FIX coding sequences in the above examples encodes a FIX protein comprising the sequence set forth in SEQ ID NO: 97. Optionally, the one of the FIX coding sequences in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

Various optimized native FIX coding sequences are also provided. In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 61-63. In another example, the one of the FIX coding sequences is (or comprises a sequence) at least 95%, at least 96%, at least 97%, at least 98%, at least 99%, at least 99.5%, or 100% identical to any one of SEQ ID NOS: 61-63. In another example, the one of the FIX coding sequences is (or comprises a sequence) at least 99%, at least 99.5%, or 100% identical to any one of SEQ ID NOS: 61-63. In another example, the one of the FIX coding sequences comprises the sequence set forth in any one of SEQ ID NOS: 61-63. In another example, the one of the FIX coding sequences consists essentially of the sequence set forth in any one of SEQ ID NOS: 61-63. In another example, the one of the FIX coding sequences consists of the sequence set forth in any one of SEQ ID NOS: 61-63. The one of the FIX coding sequences can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the one of the FIX coding sequences can be CpG depleted (e.g., all but one CpG dinucleotides removed or fully CpG-depleted) and modified to mutate one or more cryptic splice donor sequences (e.g., all identified cryptic splice donor sequences). Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 97 (and, e.g., retaining the activity of native FIX). Optionally, the one of the FIX coding sequences in the above examples encodes a FIX protein comprising the sequence set forth in SEQ ID NO: 97. Optionally, the one of the FIX coding sequences in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the one of the FIX coding sequences is (or comprises a sequence) at least 95%, at least 96%, at least 97%, at least 98%, at least 99%, at least 99.5%, or 100% identical to SEQ ID NO: 61. In another example, the one of the FIX coding sequences is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the one of the FIX coding sequences is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the one of the FIX coding sequences is (or comprises a sequence) at least 99%, at least 99.5%, or 100% identical to SEQ ID NO: 61. In another example, the one of the FIX coding sequences is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the one of the FIX coding sequences is (or comprises a sequence) at least 99%, at least 99.5%, or 100% identical to SEQ ID NO: 61 and encodes a FIX protein comprising the sequence set forth in SEQ ID NO: 97. In another example, the one of the FIX coding sequences comprises the sequence set forth in SEQ ID NO: 61. In another example, the one of the FIX coding sequences consists essentially of the sequence set forth in SEQ ID NO: 61. In another example, the one of the FIX coding sequences consists of the sequence set forth in SEQ ID NO: 61. The one of the FIX coding sequences can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the one of the FIX coding sequences can be CpG depleted (e.g., all but one CpG dinucleotides removed or fully CpG-depleted) and modified to mutate one or more cryptic splice donor sequences (e.g., all identified cryptic splice donor sequences). Optionally, the one of the FIX coding sequences encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the one of the FIX coding sequences in the above examples encodes a FIX protein (or a FIX protein comprising a sequence) at least 99%, at least 99.5%, or 100% identical to SEQ ID NO: 97 (and, e.g., retaining the activity of native FIX). Optionally, the one of the FIX coding sequences in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the one of the FIX coding sequences in the above examples encodes a FIX protein consisting of the sequence set forth in SEQ ID NO: 97.

In one example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 95%, at least 96%, at least 97%, at least 98%, at least 99%, at least 99.5%, or 100% identical to SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 99%, at least 99.5%, or 100% identical to SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences is (or comprises a sequence) at least 99%, at least 99.5%, or 100% identical to SEQ ID NO: 68 and encodes a FIX protein comprising the sequence set forth in SEQ ID NO: 97. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences comprises the sequence set forth in SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences consists essentially of the sequence set forth in SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In another example, the one of the FIX coding sequences consists of the sequence set forth in SEQ ID NO: 68. The one of the FIX coding sequences can be, for example, CpG-depleted (e.g., fully CpG-depleted) and/or codon optimized. For example, the one of the FIX coding sequences can be CpG depleted (e.g., fully CpG-depleted) and codon optimized. In one example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the other FIX coding sequence is (or comprises a sequence) at least 99%, at least 99.5%, or 100% identical to SEQ ID NO: 61. In another example, the other FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the other FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the other FIX coding sequence comprises the sequence set forth in SEQ ID NO: 61. In another example, the other FIX coding sequence consists essentially of the sequence set forth in SEQ ID NO: 61. In another example, the other FIX coding sequence consists of the sequence set forth in SEQ ID NO: 61. The other FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the other FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, one or both of the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, one or both of the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, one or both of the FIX coding sequence in the above examples encodes a FIX protein comprising the sequence set forth in SEQ ID NO: 97. Optionally, one or both of the FIX coding sequence in the above examples encodes a FIX protein consisting essentially of the sequence set forth in SEQ ID NO: 97. Optionally, one or both of the FIX coding sequence in the above examples encodes a FIX protein consisting of the sequence set forth in SEQ ID NO: 97.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 101-123 or 74-96. In another particular example, an exemplary bidirectional construct comprises a sequence at least 95%, at least 96%, at least 97%, at least 98%, at least 99%, at least 99.5%, or 100% identical to any one of SEQ ID NOS: 101-123 or 74-96. In another particular example, an exemplary bidirectional construct comprises a sequence at least 99%, at least 99.5%, or 100% identical to any one of SEQ ID NOS: 101-123 or 74-96. In another particular example, an exemplary bidirectional construct comprises any one of SEQ ID NOS: 101-123 or 74-96. In another particular example, an exemplary bidirectional construct consists essentially of any one of SEQ ID NOS: 101-123 or 74-96. In another particular example, an exemplary bidirectional construct consists of any one of SEQ ID NOS: 101-123 or 74-96.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 102-123 or 75-96. In another particular example, an exemplary bidirectional construct comprises a sequence at least 95%, at least 96%, at least 97%, at least 98%, at least 99%, at least 99.5%, or 100% identical to any one of SEQ ID NOS: 102-123 or 75-96. In another particular example, an exemplary bidirectional construct comprises a sequence at least 99%, at least 99.5%, or 100% identical to any one of SEQ ID NOS: 102-123 or 75-96. In another particular example, an exemplary bidirectional construct comprises any one of SEQ ID NOS: 102-123 or 75-96. In another particular example, an exemplary bidirectional construct consists essentially of any one of SEQ ID NOS: 102-123 or 75-96. In another particular example, an exemplary bidirectional construct consists of any one of SEQ ID NOS: 102-123 or 75-96.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to any one of SEQ ID NOS: 103-123 or 76-96. In another particular example, an exemplary bidirectional construct comprises a sequence at least 95%, at least 96%, at least 97%, at least 98%, at least 99%, at least 99.5%, or 100% identical to any one of SEQ ID NOS: 103-123 or 76-96. In another particular example, an exemplary bidirectional construct comprises a sequence at least 99%, at least 99.5%, or 100% identical to any one of SEQ ID NOS: 103-123 or 76-96. In another particular example, an exemplary bidirectional construct comprises any one of SEQ ID NOS: 103-123 or 76-96. In another particular example, an exemplary bidirectional construct consists essentially of any one of SEQ ID NOS: 103-123 or 76-96. In another particular example, an exemplary bidirectional construct consists of any one of SEQ ID NOS: 103-123 or 76-96.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 109 or 108 or 82 or 81. In another particular example, an exemplary bidirectional construct comprises a sequence at least 95%, at least 96%, at least 97%, at least 98%, at least 99%, at least 99.5%, or 100% identical to SEQ ID NO: 109 or 108 or 82 or 81. In another particular example, an exemplary bidirectional construct comprises a sequence at least 99%, at least 99.5%, or 100% identical to SEQ ID NO: 109 or 108 or 82 or 81. In another particular example, an exemplary bidirectional construct comprises SEQ ID NO: 109 or 108 or 82 or 81. In another particular example, an exemplary bidirectional construct consists essentially of SEQ ID NO: 109 or 108 or 82 or 81. In another particular example, an exemplary bidirectional construct consists of SEQ ID NO: 109 or 108 or 82 or 81.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 109 or 82. In another particular example, an exemplary bidirectional construct comprises a sequence at least 95%, at least 96%, at least 97%, at least 98%, at least 99%, at least 99.5%, or 100% identical to SEQ ID NO: 109 or 82. In another particular example, an exemplary bidirectional construct comprises a sequence at least 99%, at least 99.5%, or 100% identical to SEQ ID NO: 109 or 82. In another particular example, an exemplary bidirectional construct comprises SEQ ID NO: 109 or 82. In another particular example, an exemplary bidirectional construct consists essentially of SEQ ID NO: 109 or 82. In another particular example, an exemplary bidirectional construct consists of SEQ ID NO: 109 or 82.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 109. In another particular example, an exemplary bidirectional construct comprises a sequence at least 95%, at least 96%, at least 97%, at least 98%, at least 99%, at least 99.5%, or 100% identical to SEQ ID NO: 109. In another particular example, an exemplary bidirectional construct comprises a sequence at least 99%, at least 99.5%, or 100% identical to SEQ ID NO: 109. In another particular example, an exemplary bidirectional construct comprises SEQ ID NO: 109. In another particular example, an exemplary bidirectional construct consists essentially of SEQ ID NO: 109. In another particular example, an exemplary bidirectional construct consists of SEQ ID NO: 109.

In a particular example, an exemplary bidirectional construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 82. In another particular example, an exemplary bidirectional construct comprises a sequence at least 95%, at least 96%, at least 97%, at least 98%, at least 99%, at least 99.5%, or 100% identical to SEQ ID NO: 82. In another particular example, an exemplary bidirectional construct comprises a sequence at least 99%, at least 99.5%, or 100% identical to SEQ ID NO: 82. In another particular example, an exemplary bidirectional construct comprises SEQ ID NO: 82. In another particular example, an exemplary bidirectional construct consists essentially of SEQ ID NO: 82. In another particular example, an exemplary bidirectional construct consists of SEQ ID NO: 82.

The F9 nucleic acid constructs disclosed herein can be unidirectional constructs.

When specific unidirectional construct sequences are disclosed herein, they are meant to encompass the sequence disclosed or the reverse complement of the sequence. For example, if a unidirectional construct disclosed herein consists of the hypothetical sequence 5′-CTGGACCGA-3′, it is also meant to encompass the reverse complement of that sequence (5′-TCGGTCCAG-3′). Likewise, when unidirectional construct elements are disclosed herein in a specific 5′ to 3′ order, they are also meant to encompass the reverse complement of the order of those elements. One reason for this is that, in many embodiments disclosed herein, the unidirectional constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the unidirectional constructs, the FIX coding sequence can be a wild type FIX coding sequence without further modification. In the unidirectional constructs, the FIX coding sequence can be codon-optimized for expression in a host cell. For example, the FIX coding sequence can be codon optimized or may use one or more alternative codons for one or more amino acids of the FIX (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The unidirectional constructs disclosed herein can be modified to include any suitable structural feature as needed for any particular use and/or that confers one or more desired functions. For example, the unidirectional nucleic acid constructs disclosed herein need not comprise a homology arm and/or can be, for example, homology-independent donor constructs.

In some cases, the unidirectional nucleic acid construct does not comprise a promoter that drives the expression of FIX. For example, the expression of FIX can be driven by a promoter of the host cell (e.g., the endogenous ALB promoter when the transgene is integrated into a host cell's ALB locus). In other cases, the unidirectional nucleic acid construct can comprise one or more promoters operably linked to the FIX coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some unidirectional constructs can comprise a promoter that drives expression of the FIX coding sequence.

The unidirectional constructs can, in some cases, comprise one or more polyadenylation tail sequences or polyadenylation signal sequences. Some unidirectional constructs can comprise a polyadenylation signal sequence located 3′ of the FIX coding sequence. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a CpG-depleted BGH polyadenylation signal. For example, the polyadenylation signal can be an SV40 polyadenylation signal or a CpG-depleted BGH polyadenylation signal. In a specific example, the polyadenylation signal can comprise, consist essentially of, or consist of SEQ ID NO: 98. In another specific example, the polyadenylation signal can comprise, consist essentially of, or consist of SEQ ID NO: 99.

Methods of designing a suitable polyadenylation tail sequence are known. For example, some unidirectional constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the FIX coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The unidirectional constructs can, in some cases, comprise one or more splice acceptor sites. Some unidirectional constructs comprise a splice acceptor site located 5′ of the FIX coding sequence. In a specific example, the splice acceptor is a mouse Alb exon 2 splice acceptor. In a specific example, the splice acceptor can comprise, consist essentially of, or consist of SEQ ID NO: 100.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a F9 splice acceptor (e.g., the F9 splice acceptor used in the splicing together of exons 1 and 2 of F9). For example, such a splice acceptor can be derived from the human F9 gene. Alternatively, such a splice acceptor can be derived from the mouse F9 gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The unidirectional constructs can be circular or linear. For example, a unidirectional construct can be linear.

The unidirectional constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the unidirectional construct is single-stranded (e.g., single-stranded DNA).

The unidirectional constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the constructs disclosed herein can comprise one, two, or three ITRs or can comprise no more than two ITRs. Various methods of structural modifications are known.

Similarly, one or both ends of the construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the unidirectional constructs disclosed herein can be introduced into a cell as part of a vector having additional sequences such as, for example, replication origins, promoters, and genes encoding antibiotic resistance. The constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The FIX coding sequences in the unidirectional constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a FIX coding sequence in a unidirectional construct disclosed herein has one or more cryptic splice sites mutated or removed. In another example, a FIX coding sequence in a unidirectional construct disclosed herein has all identified cryptic splice sites mutated or removed. In another example, a FIX coding sequence in a unidirectional construct disclosed herein has one or more CpG dinucleotides removed (i.e., is CpG depleted). In another example, a FIX coding sequence in a unidirectional construct disclosed herein has all but one CpG dinucleotides removed. In another example, a FIX coding sequence in a unidirectional construct disclosed herein has all CpG dinucleotides removed (i.e., is fully CpG depleted). In another example, a FIX coding sequence in a unidirectional construct disclosed herein is codon optimized (e.g., codon optimized for expression in a human or mammal). In a specific example, a FIX coding sequence in a unidirectional construct disclosed herein has one or more CpG dinucleotides removed (i.e., is CpG depleted) and has one or more cryptic splice sites mutated or removed. In another specific example, a FIX coding sequence in a unidirectional construct disclosed herein has all but one CpG dinucleotides removed and has one or more or all identified cryptic splice sites mutated or removed. In another specific example, a FIX coding sequence in a unidirectional construct disclosed herein has one or more CpG dinucleotides removed (i.e., is CpG depleted) and is codon optimized (e.g., codon optimized for expression in a human or mammal). In another specific example, a FIX coding sequence in a unidirectional construct disclosed herein has all CpG dinucleotides removed (i.e., is fully CpG depleted) and is codon optimized (e.g., codon optimized for expression in a human or mammal).

In an exemplary unidirectional construct, the construct comprises a polyadenylation signal sequence located 3′ of the FIX coding sequence, the construct comprises a splice acceptor site located 5′ of the FIX coding sequence, and the nucleic acid construct does not comprise a promoter that drives expression of the FIX protein, and optionally the nucleic acid construct does not comprise a homology arm.

In one example of a unidirectional construct, the FIX protein coding sequence is CpG-depleted (e.g., fully CpG-depleted) and/or codon optimized (e.g., CpG-depleted and codon optimized or fully CpG-depleted and codon optimized). In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 64-73. See, e.g., WO 2023/077012 and US 2023-0149563, each of which is herein incorporated by reference in its entirety for all purposes. In another example, the FIX coding sequence is (or comprises a sequence) at least 95%, at least 96%, at least 97%, at least 98%, at least 99%, at least 99.5%, or 100% identical to any one of SEQ ID NOS: 64-73. In another example, the FIX coding sequence is (or comprises a sequence) at least 99%, at least 99.5%, or 100% identical to any one of SEQ ID NOS: 64-73. In another example, the FIX coding sequence comprises the sequence set forth in any one of SEQ ID NOS: 64-73. In another example, the FIX coding sequence consists essentially of the sequence set forth in any one of SEQ ID NOS: 64-73. In another example, the FIX coding sequence consists of the sequence set forth in any one of SEQ ID NOS: 64-73. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 66-73. In another example, the FIX coding sequence is (or comprises a sequence) at least 95%, at least 96%, at least 97%, at least 98%, at least 99%, at least 99.5%, or 100% identical to any one of SEQ ID NOS: 66-73. In another example, the FIX coding sequence is (or comprises a sequence) at least 99%, at least 99.5%, or 100% identical to any one of SEQ ID NOS: 66-73. In another example, the FIX coding sequence comprises the sequence set forth in any one of SEQ ID NOS: 66-73. In another example, the FIX coding sequence consists essentially of the sequence set forth in any one of SEQ ID NOS: 66-73. In another example, the FIX coding sequence consists of the sequence set forth in any one of SEQ ID NOS: 66-73. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 or 67. In another example, the FIX coding sequence is (or comprises a sequence) at least 95%, at least 96%, at least 97%, at least 98%, at least 99%, at least 99.5%, or 100% identical to SEQ ID NO: 68 or 67. In another example, the FIX coding sequence is (or comprises a sequence) at least 99%, at least 99.5%, or 100% identical to SEQ ID NO: 68 or 67. In another example, the FIX coding sequence comprises the sequence set forth in SEQ ID NO: 68 or 67. In another example, the FIX coding sequence consists essentially of the sequence set forth in SEQ ID NO: 68 or 67. In another example, the FIX coding sequence consists of the sequence set forth in SEQ ID NO: 68 or 67. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 68. In another example, the FIX coding sequence is (or comprises a sequence) at least 95%, at least 96%, at least 97%, at least 98%, at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 68 and encodes a FIX protein comprising the sequence set forth in SEQ ID NO: 97. In another example, the FIX coding sequence is (or comprises a sequence) at least 99%, at least 99.5%, or 100% identical to SEQ ID NO: 68. In another example, the FIX coding sequence is (or comprises a sequence) at least 99%, at least 99.5%, or 100% identical to SEQ ID NO: 68 and encodes a FIX protein (or a FIX protein comprising a sequence) at least 99%, at least 99.5%, or 100% identical to SEQ ID NO: 97. In another example, the FIX coding sequence is (or comprises a sequence) at least 99%, at least 99.5%, or 100% identical to SEQ ID NO: 68 and encodes a FIX protein comprising the sequence set forth in SEQ ID NO: 97. In another example, the FIX coding sequence comprises the sequence set forth in SEQ ID NO: 68. In another example, the FIX coding sequence consists essentially of the sequence set forth in SEQ ID NO: 68. In another example, the FIX coding sequence consists of the sequence set forth in SEQ ID NO: 68. The FIX coding sequence can be, for example, CpG-depleted (e.g., fully CpG-depleted) and/or codon optimized. For example, the FIX coding sequence can be CpG depleted (e.g., fully CpG-depleted) and codon optimized. Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

Various optimized native FIX coding sequences are also provided. In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 61-63. In another example, the FIX coding sequence is (or comprises a sequence) at least 95%, at least 96%, at least 97%, at least 98%, at least 99%, at least 99.5%, or 100% identical to any one of SEQ ID NOS: 61-63. In another example, the FIX coding sequence is (or comprises a sequence) at least 99%, at least 99.5%, or 100% identical to any one of SEQ ID NOS: 61-63. In another example, the FIX coding sequence comprises the sequence set forth in any one of SEQ ID NOS: 61-63. In another example, the FIX coding sequence consists essentially of the sequence set forth in any one of SEQ ID NOS: 61-63. In another example, the FIX coding sequence consists of the sequence set forth in any one of SEQ ID NOS: 61-63. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

In one example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the FIX coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 61. In another example, the FIX coding sequence is (or comprises a sequence) at least 95%, at least 96%, at least 97%, at least 98%, at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the FIX coding sequence is (or comprises a sequence) at least 95%, at least 96%, at least 97%, at least 98%, at least 99%, at least 99.5%, or 100% identical to SEQ ID NO: 61 and encodes a FIX protein comprising the sequence set forth in SEQ ID NO: 97. In another example, the FIX coding sequence is (or comprises a sequence) at least 99%, at least 99.5%, or 100% identical to SEQ ID NO: 61. In another example, the FIX coding sequence is (or comprises a sequence) at least 99%, at least 99.5%, or 100% identical to SEQ ID NO: 61 and encodes a FIX protein (or a FIX protein comprising a sequence) at least 99%, at least 99.5%, or 100% identical to SEQ ID NO: 97. In another example, the FIX coding sequence is (or comprises a sequence) at least 99%, at least 99.5%, or 100% identical to SEQ ID NO: 61 and encodes a FIX protein comprising the sequence set forth in SEQ ID NO: 97. In another example, the FIX coding sequence comprises the sequence set forth in SEQ ID NO: 61. In another example, the FIX coding sequence consists essentially of the sequence set forth in SEQ ID NO: 61. In another example, the one of the FIX coding sequences consists of the sequence set forth in SEQ ID NO: 61. The FIX coding sequence can be, for example, CpG-depleted (e.g., all but one CpG dinucleotides removed or fully CpG-depleted) and/or modified to mutate one or more cryptic splice donor sequences (e.g., all identified cryptic splice donor sequences). For example, the FIX coding sequence can be CpG depleted (e.g., all but one CpG dinucleotides removed or fully CpG-depleted) and modified to mutate one or more cryptic splice donor sequences (e.g., all identified cryptic splice donor sequences). Optionally, the FIX coding sequence encodes a FIX protein (or a FIX protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence encodes a FIX protein (or a FIX protein comprising a sequence) at least 95%, at least 96%, at least 97%, at least 98%, at least 99%, at least 99.5%, or 100% identical to SEQ ID NO: 97 (and, e.g., retaining the activity of native FIX). Optionally, the FIX coding sequence in the above examples encodes a FIX protein (or a FIX protein comprising a sequence) at least 99%, at least 99.5%, or 100% identical to SEQ ID NO: 97 (and, e.g., retaining the activity of native FIX). Optionally, the FIX coding sequence in the above examples encodes a FIX protein comprising the sequence set forth in SEQ ID NO: 97. Optionally, the FIX coding sequence in the above examples encodes a FIX protein consisting essentially of the sequence set forth in SEQ ID NO: 97. Optionally, the FIX coding sequence in the above examples encodes a FIX protein consisting of the sequence set forth in SEQ ID NO: 97.

### (8) Multidomain Therapeutic Protein Nucleic Acid Constructs

The multidomain therapeutic protein nucleic acid constructs disclosed herein can be unidirectional constructs or bidirectional constructs. Examples of such constructs are provided in PCT/US2023/061858 and U.S. Ser. No. 18/163,698, each of which is herein incorporated by reference in its entirety for all purposes. When specific construct sequences are disclosed herein, they are meant to encompass the sequence disclosed or the reverse complement of the sequence. For example, if a construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

In the nucleic acid constructs, the multidomain therapeutic protein coding sequence, the CD63-binding delivery domain coding sequence, and/or the GAA coding sequence can be codon-optimized for expression in a host cell. For example, the multidomain therapeutic protein coding sequence, the CD63-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

In the nucleic acid constructs, the multidomain therapeutic protein coding sequence, the TfR-binding delivery domain coding sequence, and/or the GAA coding sequence can be codon-optimized for expression in a host cell. For example, the multidomain therapeutic protein coding sequence, the TfR-binding delivery domain coding sequence, and/or the GAA coding sequence can be codon optimized or may use one or more alternative codons for one or more amino acids of the protein (i.e., same amino acid sequence). An alternative codon as used herein refers to variations in codon usage for a given amino acid, and may or may not be a preferred or optimized codon (codon optimized) for a given expression system. Preferred codon usage, or codons that are well-tolerated in a given system of expression, are known.

The nucleic acid constructs disclosed herein can be modified to include any suitable structural feature as needed for any particular use and/or that confers one or more desired functions. For example, the nucleic acid constructs disclosed herein need not comprise a homology arm and/or can be, for example, homology-independent donor constructs.

In some cases, the nucleic acid construct does not comprise a promoter that drives the expression of the multidomain therapeutic protein. For example, the expression of the multidomain therapeutic protein can be driven by a promoter of the host cell (e.g., the endogenous ALB promoter when the transgene is integrated into a host cell's ALB locus). In other cases, the nucleic acid construct can comprise one or more promoters operably linked to the multidomain therapeutic protein coding sequence. That is, although not required for expression, the constructs disclosed herein may also include transcriptional or translational regulatory sequences such as promoters, enhancers, insulators, internal ribosome entry sites, additional sequences encoding peptides, and/or polyadenylation signals. Some nucleic acid constructs can comprise a promoter that drives expression of the multidomain therapeutic protein. For example, the promoter may be a liver-specific promoter. Examples of liver-specific promoters include TTR promoters, such as human or mouse TTR promoters. In one example, the construct may comprise a TTR promoter, such as a mouse TTR promoter or a human TTR promoter (e.g., the coding sequence for the multidomain therapeutic protein is operably linked to the TTR promoter). In one example, the construct may comprise a SERPINA1 enhancer, such as a mouse SERPINA1 enhancer or a human SERPINA1 enhancer (e.g., the coding sequence for the multidomain therapeutic protein is operably linked to the SERPINA1 enhancer). In one example, the construct may comprise a TTR promoter and a SERPINA1 enhancer, such as a human SERPINA1 enhancer and a mouse TTR promoter (e.g., the coding sequence for the multidomain therapeutic protein is operably linked to the SERPINA1 enhancer and the TTR promoter).

The nucleic acid constructs can, in some cases, comprise one or more polyadenylation tail sequences or polyadenylation signal sequences. Some nucleic acid constructs can comprise a polyadenylation signal sequence located 3′ of the multidomain therapeutic protein coding sequence. In a specific example, the polyadenylation signal is a simian virus 40 (SV40) late polyadenylation signal (or a variant thereof). In another specific example, the polyadenylation signal is a bovine growth hormone (BGH) polyadenylation signal (or a variant thereof). In another specific example, the polyadenylation signal is a CpG-depleted BGH polyadenylation signal. For example, the polyadenylation signal can be an SV40 polyadenylation signal or a CpG-depleted BGH polyadenylation signal. For example, the polyadenylation signal can comprise, consist essentially of, or consist of SEQ ID NO: 827, 292, or 284. In a specific example, the polyadenylation signal can comprise, consist essentially of, or consist of SEQ ID NO: 827. In a specific example, the polyadenylation signal can comprise, consist essentially of, or consist of SEQ ID NO: 292. In another specific example, the polyadenylation signal can comprise, consist essentially of, or consist of SEQ ID NO: 284. In another specific example, the polyadenylation signal can comprise, consist essentially of, or consist of SEQ ID NO: 285.

In one example, the polyadenylation signal can comprise a BGH polyadenylation signal. For example, the BGH polyadenylation signal can comprise, consist essentially of, or consist of SEQ ID NO: 858. In another example, the polyadenylation signal can comprise an SV40 polyadenylation signal. For example, the SV40 polyadenylation signal can be a unidirectional SV40 late polyadenylation signal. For example, the transcription terminator sequences that are present in the “early” inverse orientation of SV40 can be mutated (e.g., by mutating the reverse strand AAUAAA sequences to AAUCAA). The SV40 polyA is bidirectional, but the polyadenylation in the “late” orientation is more efficient than the polyadenylation in the “early” orientation. For example, the unidirectional SV40 late polyadenylation signal can comprise, consist essentially of, or consist of SEQ ID NO: 859. In another example, a synthetic polyadenylation signal can be used. For example, the synthetic polyadenylation signal can comprise, consist essentially of, or consist of SEQ ID NO: 860. In another example, two or more polyadenylation signals can be used in combination. For example, the polyadenylation signal can comprise a combination of a BGH polyadenylation signal and an SV40 polyadenylation signal (e.g., an SV40 late polyadenylation signal, such as a unidirectional SV40 late polyadenylation signal). For example, the polyadenylation signal can comprise a combination of a BGH polyadenylation signal and a unidirectional SV40 late polyadenylation signal. For example, the BGH polyadenylation signal can comprise, consist essentially of, or consist of SEQ ID NO: 858, and the unidirectional SV40 late polyadenylation signal can comprise, consist essentially of, or consist of SEQ ID NO: 859. In a specific example, the BGH polyadenylation signal can be upstream (5′) of the SV40 polyadenylation signal (e.g., unidirectional SV40 late polyadenylation signal). For example, the combined polyadenylation signal can comprise the sequence set forth in SEQ ID NO: 902. In another example, the polyadenylation signal can comprise a combination of a BGH polyadenylation signal and a synthetic polyadenylation signal. For example, the BGH polyadenylation signal can comprise, consist essentially of, or consist of SEQ ID NO: 858, and the synthetic polyadenylation signal can comprise, consist essentially of, or consist of SEQ ID NO: 860. In some embodiments, the nucleic acid construct is a unidirectional construct.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

In some embodiments, unidirectional SV40 late polyadenylation signals are used. The SV40 polyA is bidirectional, but the polyadenylation in the “late” orientation is more efficient than the polyadenylation in the “early” orientation. The unidirectional SV40 late polyadenylation signals described herein are positioned in the “late” orientation, with the polyadenylation signals present in the “early” orientation mutated or inactivated. In some embodiments, each instance of the sequence AATAAA in the reverse strand is mutated in the unidirectional SV40 late polyadenylation signal. For example, the two conserved AATAAA poly(A) signals present in the SV40 “early” poly(A) to AATCAA. In some embodiments, the unidirectional SV40 late polyadenylation signal is at least 95%, at least 96%, at least 97%, at least 98%, or at least 99% identical to the sequence set forth in SEQ ID NO: 859. In some embodiments, the unidirectional SV40 late polyadenylation signal comprises, consists essentially of, or consists of the sequence set forth in SEQ ID NO: 859.

The unidirectional SV40 late polyadenylation signals can be used in combination with (e.g., in tandem with) one or more additional polyadenylation signals. Examples of transcription terminators that can be used include, for example, the human growth hormone (HGH) polyadenylation signal, the simian virus 40 (SV40) late polyadenylation signal, the rabbit beta-globin polyadenylation signal, the bovine growth hormone (BGH) polyadenylation signal, the phosphoglycerate kinase (PGK) polyadenylation signal, an AOX1 transcription termination sequence, a CYC1 transcription termination sequence, or any transcription termination sequence known to be suitable for regulating gene expression in eukaryotic cells. For example, the unidirectional SV40 late polyadenylation signals can be used in combination with (e.g., in tandem with) a bovine growth hormone (BGH) polyadenylation signal, optionally wherein the BGH polyadenylation signal is upstream of (5′ of) the unidirectional SV40 late polyadenylation signal. In some embodiments, the BGH polyadenylation signal is at least 95%, at least 96%, at least 97%, at least 98%, or at least 99% identical to the sequence set forth in SEQ ID NO: 858. In some embodiments, the BGH polyadenylation signal comprises, consists essentially of, or consists of the sequence set forth in SEQ ID NO: 858. In some embodiments, the combination of the BGH polyadenylation signal and the unidirectional SV40 late polyadenylation signal is at least 95%, at least 96%, at least 97%, at least 98%, or at least 99% identical to the sequence set forth in SEQ ID NO: 902. In some embodiments, the combination of the BGH polyadenylation signal and the unidirectional SV40 late polyadenylation signal comprises, consists essentially of, or consists of the sequence set forth in SEQ ID NO: 902.

In some embodiments, a stuffer sequence can be used to increase the time between when RNA polymerase transcribes the polyA to the time when it transcribes the next splice acceptor. For example, the stuffer sequence can be used between two different polyadenylation signals (e.g., between a BGH polyadenylation signal and a synthetic polyadenylation signal. For example, the stuffer sequence can comprise, consist essentially of, or consist of SEQ ID NO: 861.

In some embodiments, MAZ elements that cause polymerase pausing are used in combination with a polyadenylation signal (e.g., a BGH polyadenylation signal or an SV40 polyadenylation signal). For example, one or more (e.g., at least 1, at least 2, at least 3, at least 4, or about 1 to about 4, about 2 to about 4, about 3 to about 4, or 1, 2, 3, or 4) MAZ elements can be used in combination with a polyadenylation signal. For example, the MAZ element can comprise, consist essentially of, or consist of SEQ ID NO: 862.

Methods of designing a suitable polyadenylation tail sequence are known. For example, some nucleic acid constructs comprise a polyadenylation tail sequence and/or a polyadenylation signal sequence downstream of an open reading frame (i.e., a polyadenylation tail sequence and/or a polyadenylation signal sequence 3′ of a coding sequence). The polyadenylation tail sequence can be encoded, for example, as a “poly-A” stretch downstream of the multidomain therapeutic protein coding sequence (or other protein coding sequence) in the first and/or second segment. A poly-A tail can comprise, for example, at least 20, 30, 40, 50, 60, 70, 80, 90, or 100 adenines, and optionally up to 300 adenines. In a specific example, the poly-A tail comprises 95, 96, 97, 98, 99, or 100 adenine nucleotides. Methods of designing a suitable polyadenylation tail sequence and/or polyadenylation signal sequence are well known. For example, the polyadenylation signal sequence AAUAAA is commonly used in mammalian systems, although variants such as UAUAAA or AU/GUAAA have been identified. See, e.g., Proudfoot (2011) Genes & Dev. 25(17):1770-82, herein incorporated by reference in its entirety for all purposes.

The nucleic acid constructs can, in some cases, comprise one or more splice acceptor sites. Some nucleic acid constructs comprise a splice acceptor site located 5′ of the multidomain therapeutic protein coding sequence. In a specific example, the splice acceptor is a mouse Alb exon 2 splice acceptor. In a specific example, the splice acceptor can comprise, consist essentially of, or consist of SEQ ID NO: 286.

The splice acceptor site can, for example, comprise NAG or consist of NAG. In a specific example, the splice acceptor is an ALB splice acceptor (e.g., an ALB splice acceptor used in the splicing together of exons 1 and 2 of ALB (i.e., ALB exon 2 splice acceptor)). For example, such a splice acceptor can be derived from the human ALB gene. In another example, the splice acceptor can be derived from the mouse Alb gene (e.g., an ALB splice acceptor used in the splicing together of exons 1 and 2 of mouse Alb (i.e., mouse Alb exon 2 splice acceptor)). In another example, the splice acceptor is a GAA splice acceptor. For example, such a splice acceptor can be derived from the human GAA gene. Alternatively, such a splice acceptor can be derived from the mouse GAA gene. Additional suitable splice acceptor sites useful in eukaryotes, including artificial splice acceptors, are known. See, e.g., Shapiro et al. (1987) Nucleic Acids Res. 15:7155-7174 and Burset et al. (2001) Nucleic Acids Res. 29:255-259, each of which is herein incorporated by reference in its entirety for all purposes.

The nucleic acid constructs can be circular or linear. For example, a nucleic acid construct can be linear. The nucleic acid constructs disclosed herein can be DNA or RNA, single-stranded, double-stranded, or partially single-stranded and partially double-stranded. For example, the constructs can be single- or double-stranded DNA. In some embodiments, the nucleic acid can be modified (e.g., using nucleoside analogs), as described herein. In a specific example, the nucleic acid construct is single-stranded (e.g., single-stranded DNA).

The nucleic acid constructs disclosed herein can be modified on either or both ends to include one or more suitable structural features as needed and/or to confer one or more functional benefit. For example, structural modifications can vary depending on the method(s) used to deliver the constructs disclosed herein to a host cell (e.g., use of viral vector delivery or packaging into lipid nanoparticles for delivery). Such modifications include, for example, terminal structures such as inverted terminal repeats (ITR), hairpin, loops, and other structures such as toroids. For example, the nucleic acid constructs disclosed herein can comprise one, two, or three ITRs or can comprise no more than two ITRs. Various methods of structural modifications are known.

Similarly, one or both ends of the nucleic acid construct can be protected (e.g., from exonucleolytic degradation) by known methods. For example, one or more dideoxynucleotide residues can be added to the 3′ terminus of a linear molecule and/or self-complementary oligonucleotides can be ligated to one or both ends. See, e.g., Chang et al. (1987) Proc. Natl. Acad. Sci. U.S.A. 84:4959-4963 and Nehls et al. (1996) Science 272:886-889, each of which is herein incorporated by reference in its entirety for all purposes. Additional methods for protecting the constructs from degradation include, but are not limited to, addition of terminal amino group(s) and the use of modified internucleotide linkages such as, for example, phosphorothioates, phosphoramidates, and O-methyl ribose or deoxyribose residues.

As disclosed in more detail herein, the nucleic acid constructs disclosed herein can be introduced into a cell as part of a vector having additional sequences such as, for example, replication origins, promoters, and genes encoding antibiotic resistance. The nucleic acid constructs can be introduced as a naked nucleic acid, can be introduced as a nucleic acid complexed with an agent such as a liposome, polymer, or poloxamer, or can be delivered by viral vectors (e.g., adenovirus, AAV, herpesvirus, retrovirus, lentivirus).

The multidomain therapeutic protein coding sequence, the CD63-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a CD63-binding delivery domain coding sequence, and/or a GAA coding sequence in a nucleic acid construct disclosed herein has one or more cryptic splice sites mutated or removed. In another example, a multidomain therapeutic protein coding sequence, a CD63-binding delivery domain coding sequence, and/or a GAA coding sequence in a nucleic acid construct disclosed herein has all identified cryptic splice sites mutated or removed. In another example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a CD63-binding delivery domain coding sequence, and/or a GAA coding sequence in a nucleic acid construct disclosed herein has all CpG dinucleotides removed. In another example, a multidomain therapeutic protein coding sequence, a CD63-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a CD63-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a CD63-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a CD63-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

The multidomain therapeutic protein coding sequence, the TfR-binding delivery domain coding sequence, and/or the GAA coding sequence in the nucleic acid constructs disclosed herein may include one or more modifications such as codon optimization (e.g., to human codons), depletion of CpG dinucleotides, mutation of cryptic splice sites, addition of one or more glycosylation sites, or any combination thereof. CpG dinucleotides in a construct can limit the therapeutic utility of the construct. First, unmethylated CpG dinucleotides can interact with host toll-like receptor-9 (TLR-9) to stimulate innate, proinflammatory immune responses. Second, once the CpG dinucleotides become methylated, they can result in the suppression of transgene expression coordinated by methyl-CpG binding proteins. Cryptic splice sites are sequences in a pre-messenger RNA that are not normally used as splice sites, but that can be activated, for example, by mutations that either inactivate canonical splice sites or create splice sites where one did not exist before. Accurate splice site selection is critical for successful gene expression, and removal of cryptic splice sites can favor use of the normal or intended splice site.

In one example, a multidomain therapeutic protein coding sequence, a TfR-binding delivery domain coding sequence, and/or a GAA coding sequence in a nucleic acid construct disclosed herein has one or more cryptic splice sites mutated or removed. In another example, a multidomain therapeutic protein coding sequence, a TfR-binding delivery domain coding sequence, and/or a GAA coding sequence in a nucleic acid construct disclosed herein has all identified cryptic splice sites mutated or removed. In another example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted). In another example, a multidomain therapeutic protein coding sequence, a TfR-binding delivery domain coding sequence, and/or a GAA coding sequence in a nucleic acid construct disclosed herein has all CpG dinucleotides removed. In another example, a multidomain therapeutic protein coding sequence, a TfR-binding delivery domain coding sequence, and/or a GAA coding sequence in a nucleic acid construct disclosed herein is codon optimized (e.g., codon optimized for expression in a human or mammal). In a specific example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted) and has one or more cryptic splice sites mutated or removed. In another specific example, a multidomain therapeutic protein coding sequence, a TfR-binding delivery domain coding sequence, and/or a GAA coding sequence in a nucleic acid construct disclosed herein has all CpG dinucleotides removed and has one or more or all identified cryptic splice sites mutated or removed. In another specific example, a multidomain therapeutic protein coding sequence, a TfR-binding delivery domain coding sequence, and/or a GAA coding sequence in a nucleic acid construct disclosed herein has one or more CpG dinucleotides removed (i.e., is CpG depleted) and is codon optimized (e.g., codon optimized for expression in a human or mammal). In another specific example, a multidomain therapeutic protein coding sequence, a TfR-binding delivery domain coding sequence, and/or a GAA coding sequence in a nucleic acid construct disclosed herein has all CpG dinucleotides removed (i.e., is fully CpG depleted) and is codon optimized (e.g., codon optimized for expression in a human or mammal).

In an exemplary nucleic acid construct, the construct comprises a polyadenylation signal sequence located 3′ of the multidomain therapeutic protein coding sequence, the construct comprises a splice acceptor site located 5′ of the multidomain therapeutic protein coding sequence, and the nucleic acid construct does not comprise a promoter that drives expression of the multidomain therapeutic protein, and optionally the nucleic acid construct does not comprise a homology arm.

In a specific example of a multidomain therapeutic protein nucleic acid construct, the encoded multidomain therapeutic protein can comprise SEQ ID NO: 853 or can be at least 90%, at least 91%, at least 92%, at least 93%, at least 94%, at least 95%, at least 96%, at least 97%, at least 98%, at least 99%, or at least 99.5% identical to SEQ ID NO: 853. In another specific example, the multidomain therapeutic protein can consist essentially of SEQ ID NO: 853. In another specific example, the multidomain therapeutic protein can consist of SEQ ID NO: 853.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 852 or 704. In another example, the multidomain therapeutic protein coding sequence is (or comprises a sequence) at least 95%, at least 96%, at least 97%, at least 98%, at least 99%, at least 99.5%, or 100% identical to SEQ ID NO: 852 or 704. In another example, the multidomain therapeutic protein coding sequence is (or comprises a sequence) at least 99%, at least 99.5%, or 100% identical to SEQ ID NO: 852 or 704. In another example, the multidomain therapeutic protein coding sequence comprises the sequence set forth in SEQ ID NO: 852 or 704. In another example, the multidomain therapeutic protein coding sequence consists essentially of the sequence set forth in SEQ ID NO: 852 or 704. In another example, the multidomain therapeutic protein coding sequence consists of the sequence set forth in SEQ ID NO: 852 or 704. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 853 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 853 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 853 (and, e.g., retaining the activity of native GAA). Optionally, the multidomain therapeutic protein coding sequence in the above examples encodes a multidomain therapeutic protein comprising the sequence set forth in SEQ ID NO: 853. Optionally, the multidomain therapeutic protein coding sequence in the above examples encodes a multidomain therapeutic protein consisting essentially of the sequence set forth in SEQ ID NO: 853. Optionally, the multidomain therapeutic protein coding sequence in the above examples encodes a multidomain therapeutic protein consisting of the sequence set forth in SEQ ID NO: 853. In some embodiments, the nucleotide at position 1857 is a “G.” In some embodiments, the nucleotide at position 1860 is a “C.” In some embodiments, the nucleotide at position 3105 is a “G.” In some embodiments, the nucleotide at position 1857 is a “G,” the nucleotide at position 1860 is a “C,” and the nucleotide at position 3105 is a “G.”

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 852. In another example, the multidomain therapeutic protein coding sequence is (or comprises a sequence) at least 95%, at least 96%, at least 97%, at least 98%, at least 99%, at least 99.5%, or 100% identical to SEQ ID NO: 852. In another example, the multidomain therapeutic protein coding sequence is (or comprises a sequence) at least 99%, at least 99.5%, or 100% identical to SEQ ID NO: 852. In another example, the multidomain therapeutic protein coding sequence comprises the sequence set forth in SEQ ID NO: 852. In another example, the multidomain therapeutic protein coding sequence consists essentially of the sequence set forth in SEQ ID NO: 852. In another example, the multidomain therapeutic protein coding sequence consists of the sequence set forth in SEQ ID NO: 852. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 853 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 853 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 853 (and, e.g., retaining the activity of native GAA). Optionally, the multidomain therapeutic protein coding sequence in the above examples encodes a multidomain therapeutic protein comprising the sequence set forth in SEQ ID NO: 853. Optionally, the multidomain therapeutic protein coding sequence in the above examples encodes a multidomain therapeutic protein consisting essentially of the sequence set forth in SEQ ID NO: 853. Optionally, the multidomain therapeutic protein coding sequence in the above examples encodes a multidomain therapeutic protein consisting of the sequence set forth in SEQ ID NO: 853. In some embodiments, the nucleotide at position 1857 is a “G.” In some embodiments, the nucleotide at position 1860 is a “C.” In some embodiments, the nucleotide at position 3105 is a “G.” In some embodiments, the nucleotide at position 1857 is a “G,” the nucleotide at position 1860 is a “C,” and the nucleotide at position 3105 is a “G.”

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., a BGH polyadenylation signal, such as the one set forth in SEQ ID NO: 858, or a combination of a BGH polyadenylation signal and a unidirectional SV40 late polyadenylation signal, such as the ones set forth in SEQ ID NOS: 858 and 859, respectively), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 871, 872, 887, or 888. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 871, 872, 887, or 888 and encodes a multidomain therapeutic protein (or a multidomain therapeutic protein comprising a sequence) at least 99%, at least 99.5%, or 100% identical to SEQ ID NO: 853. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 871, 872, 887, or 888 and encodes a multidomain therapeutic protein comprising the sequence set forth in SEQ ID NO: 853. In another example, the nucleic acid construct comprises a sequence at least 95%, at least 96%, at least 97%, at least 98%, at least 99%, at least 99.5%, or 100% identical to SEQ ID NO: 871, 872, 887, or 888. In another example, the nucleic acid construct comprises a sequence at least 95%, at least 96%, at least 97%, at least 98%, at least 99%, at least 99.5%, or 100% identical to SEQ ID NO: 871, 872, 887, or 888 and encodes a multidomain therapeutic protein (or a multidomain therapeutic protein comprising a sequence) at least 99%, at least 99.5%, or 100% identical to SEQ ID NO: 853. In another example, the nucleic acid construct comprises a sequence at least 95%, at least 96%, at least 97%, at least 98%, at least 99%, at least 99.5%, or 100% identical to SEQ ID NO: 871, 872, 887, or 888 and encodes a multidomain therapeutic protein comprising the sequence set forth in SEQ ID NO: 853. In another example, the nucleic acid construct comprises a sequence at least 99%, at least 99.5%, or 100% identical to SEQ ID NO: 871, 872, 887, or 888. In another example, the nucleic acid construct comprises a sequence at least 99%, at least 99.5%, or 100% identical to SEQ ID NO: 871, 872, 887, or 888 and encodes a multidomain therapeutic protein (or a multidomain therapeutic protein comprising a sequence) at least 99%, at least 99.5%, or 100% identical to SEQ ID NO: 853. In another example, the nucleic acid construct comprises a sequence at least 99%, at least 99.5%, or 100% identical to SEQ ID NO: 871, 872, 887, or 888 and encodes a multidomain therapeutic protein comprising the sequence set forth in SEQ ID NO: 853. In another example, the nucleic acid construct comprises the sequence set forth in SEQ ID NO: 871, 872, 887, or 888. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 853 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 853 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 853 (and, e.g., retaining the activity of native GAA). Optionally, the nucleic acid construct in the above examples encodes a multidomain therapeutic protein comprising the sequence set forth in SEQ ID NO: 853. Optionally, the nucleic acid construct in the above examples encodes a multidomain therapeutic protein consisting essentially of the sequence set forth in SEQ ID NO: 853. Optionally, the nucleic acid construct in the above examples encodes a multidomain therapeutic protein consisting of the sequence set forth in SEQ ID NO: 853.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., a BGH polyadenylation signal, such as the one set forth in SEQ ID NO: 858, or a combination of a BGH polyadenylation signal and a unidirectional SV40 late polyadenylation signal, such as the ones set forth in SEQ ID NOS: 858 and 859, respectively), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 871 or 887. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 871 or 887 and encodes a multidomain therapeutic protein (or a multidomain therapeutic protein comprising a sequence) at least 99%, at least 99.5%, or 100% identical to SEQ ID NO: 853. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 871 or 887 and encodes a multidomain therapeutic protein comprising the sequence set forth in SEQ ID NO: 853. In another example, the nucleic acid construct comprises a sequence at least 95%, at least 96%, at least 97%, at least 98%, at least 99%, at least 99.5%, or 100% identical to SEQ ID NO: 871 or 887. In another example, the nucleic acid construct comprises a sequence at least 95%, at least 96%, at least 97%, at least 98%, at least 99%, at least 99.5%, or 100% identical to SEQ ID NO: 871 or 887 and encodes a multidomain therapeutic protein (or a multidomain therapeutic protein comprising a sequence) at least 99%, at least 99.5%, or 100% identical to SEQ ID NO: 853. In another example, the nucleic acid construct comprises a sequence at least 95%, at least 96%, at least 97%, at least 98%, at least 99%, at least 99.5%, or 100% identical to SEQ ID NO: 871 or 887 and encodes a multidomain therapeutic protein comprising the sequence set forth in SEQ ID NO: 853. In another example, the nucleic acid construct comprises a sequence at least 99%, at least 99.5%, or 100% identical to SEQ ID NO: 871 or 887. In another example, the nucleic acid construct comprises a sequence at least 99%, at least 99.5%, or 100% identical to SEQ ID NO: 871 or 887 and encodes a multidomain therapeutic protein (or a multidomain therapeutic protein comprising a sequence) at least 99%, at least 99.5%, or 100% identical to SEQ ID NO: 853. In another example, the nucleic acid construct comprises a sequence at least 99%, at least 99.5%, or 100% identical to SEQ ID NO: 871 or 887 and encodes a multidomain therapeutic protein comprising the sequence set forth in SEQ ID NO: 853. In another example, the nucleic acid construct comprises the sequence set forth in SEQ ID NO: 871 or 887. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 853 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 853 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 853 (and, e.g., retaining the activity of native GAA). Optionally, the nucleic acid construct in the above examples encodes a multidomain therapeutic protein comprising the sequence set forth in SEQ ID NO: 853. Optionally, the nucleic acid construct in the above examples encodes a multidomain therapeutic protein consisting essentially of the sequence set forth in SEQ ID NO: 853. Optionally, the nucleic acid construct in the above examples encodes a multidomain therapeutic protein consisting of the sequence set forth in SEQ ID NO: 853.

In a specific example of a multidomain therapeutic protein nucleic acid construct, the encoded multidomain therapeutic protein can comprise SEQ ID NO: 316 or can be at least 90%, at least 91%, at least 92%, at least 93%, at least 94%, at least 95%, at least 96%, at least 97%, at least 98%, at least 99%, or at least 99.5% identical to SEQ ID NO: 316. In another specific example, the multidomain therapeutic protein can consist essentially of SEQ ID NO: 316. In another specific example, the multidomain therapeutic protein can consist of SEQ ID NO: 316.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 863, 864, 865, and 319. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 863, 864, 865, and 319. In another example, the multidomain therapeutic protein coding sequence is (or comprises a sequence) at least 99%, at least 99.5%, or 100% identical to any one of SEQ ID NOS: 863, 864, 865, and 319. In another example, the multidomain therapeutic protein coding sequence comprises the sequence set forth in any one of SEQ ID NOS: 863, 864, 865, and 319. In another example, the multidomain therapeutic protein coding sequence consists essentially of the sequence set forth in any one of SEQ ID NOS: 863, 864, 865, and 319. In another example, the multidomain therapeutic protein coding sequence consists of the sequence set forth in any one of SEQ ID NOS: 863, 864, 865, and 319. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein comprising the sequence set forth in SEQ ID NO: 316. Optionally, the multidomain therapeutic protein coding sequence in the above examples encodes a multidomain therapeutic protein consisting essentially of the sequence set forth in SEQ ID NO: 316. Optionally, the multidomain therapeutic protein coding sequence in the above examples encodes a multidomain therapeutic protein consisting of the sequence set forth in SEQ ID NO: 316. In some embodiments, the nucleotide at position 3 is an “A.” In some embodiments, the nucleotide at position 132 is an “A.” In some embodiments, the nucleotide at position 273 is a “T.” In some embodiments, the nucleotide at position 723 is a “G.” In some embodiments, the nucleotide at position 1830 is a “G.” In some embodiments, the nucleotide at position 1833 is a “C.” In some embodiments, the nucleotide at position 3078 is a “G.” In some embodiments, the nucleotide at position 3 is an “A,” the nucleotide at position 132 is an “A,” the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 1830 is a “G,” the nucleotide at position 1833 is a “C,” and the nucleotide at position 3078 is a “G.”

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 863. In another example, the multidomain therapeutic protein coding sequence is (or comprises a sequence) at least 95%, at least 96%, at least 97%, at least 98%, at least 99%, at least 99.5%, or 100% identical to SEQ ID NO: 863. In another example, the multidomain therapeutic protein coding sequence is (or comprises a sequence) at least 99%, at least 99.5%, or 100% identical to SEQ ID NO: 863. In another example, the multidomain therapeutic protein coding sequence comprises the sequence set forth in SEQ ID NO: 863. In another example, the multidomain therapeutic protein coding sequence consists essentially of the sequence set forth in SEQ ID NO: 863. In another example, the multidomain therapeutic protein coding sequence consists of the sequence set forth in SEQ ID NO: 863. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein comprising the sequence set forth in SEQ ID NO: 316. Optionally, the multidomain therapeutic protein coding sequence in the above examples encodes a multidomain therapeutic protein consisting essentially of the sequence set forth in SEQ ID NO: 316. Optionally, the multidomain therapeutic protein coding sequence in the above examples encodes a multidomain therapeutic protein consisting of the sequence set forth in SEQ ID NO: 316. In some embodiments, the nucleotide at position 3 is an “A.” In some embodiments, the nucleotide at position 132 is an “A.” In some embodiments, the nucleotide at position 273 is a “T.” In some embodiments, the nucleotide at position 723 is a “G.” In some embodiments, the nucleotide at position 1830 is a “G.” In some embodiments, the nucleotide at position 1833 is a “C.” In some embodiments, the nucleotide at position 3078 is a “G.” In some embodiments, the nucleotide at position 3 is an “A,” the nucleotide at position 132 is an “A,” the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 273 is a “T,” the nucleotide at position 723 is a “G,” the nucleotide at position 1830 is a “G,” the nucleotide at position 1833 is a “C,” and the nucleotide at position 3078 is a “G.” In some embodiments, the nucleotide at position 1830 is a “G,” the nucleotide at position 1833 is a “C,” and the nucleotide at position 3078 is a “G.”

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., a BGH polyadenylation signal, such as the one set forth in SEQ ID NO: 858, or a combination of a BGH polyadenylation signal and a unidirectional SV40 late polyadenylation signal, such as the ones set forth in SEQ ID NOS: 858 and 859, respectively), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 884, 885, 900, or 901. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 884, 885, 900, or 901 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 884, 885, 900, or 901 and encodes a multidomain therapeutic protein comprising the sequence set forth in SEQ ID NO: 316. In another example, the nucleic acid construct comprises a sequence at least 95%, at least 96%, at least 97%, at least 98%, at least 99%, at least 99.5%, or 100% identical to SEQ ID NO: 884, 885, 900, or 901. In another example, the nucleic acid construct comprises a sequence at least 95%, at least 96%, at least 97%, at least 98%, at least 99%, at least 99.5%, or 100% identical to SEQ ID NO: 884, 885, 900, or 901 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 95%, at least 96%, at least 97%, at least 98%, at least 99%, at least 99.5%, or 100% identical to SEQ ID NO: 884, 885, 900, or 901 and encodes a multidomain therapeutic protein comprising the sequence set forth in SEQ ID NO: 316. In another example, the nucleic acid construct comprises a sequence at least 99%, at least 99.5%, or 100% identical to SEQ ID NO: 884, 885, 900, or 901. In another example, the nucleic acid construct comprises a sequence at least 99%, at least 99.5%, or 100% identical to SEQ ID NO: 884, 885, 900, or 901 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 99%, at least 99.5%, or 100% identical to SEQ ID NO: 884, 885, 900, or 901 and encodes a multidomain therapeutic protein comprising the sequence set forth in SEQ ID NO: 316. In another example, the nucleic acid construct comprises the sequence set forth in SEQ ID NO: 884, 885, 900, or 901. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the nucleic acid construct in the above examples encodes a multidomain therapeutic protein comprising the sequence set forth in SEQ ID NO: 316. Optionally, the nucleic acid construct in the above examples encodes a multidomain therapeutic protein consisting essentially of the sequence set forth in SEQ ID NO: 316. Optionally, the nucleic acid construct in the above examples encodes a multidomain therapeutic protein consisting of the sequence set forth in SEQ ID NO: 316.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., a BGH polyadenylation signal, such as the one set forth in SEQ ID NO: 858, or a combination of a BGH polyadenylation signal and a unidirectional SV40 late polyadenylation signal, such as the ones set forth in SEQ ID NOS: 858 and 859, respectively), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 884 or 900. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 884 or 900 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 884 or 900 and encodes a multidomain therapeutic protein comprising the sequence set forth in SEQ ID NO: 316. In another example, the nucleic acid construct comprises a sequence at least 95%, at least 96%, at least 97%, at least 98%, at least 99%, at least 99.5%, or 100% identical to SEQ ID NO: 884 or 900. In another example, the nucleic acid construct comprises a sequence at least 95%, at least 96%, at least 97%, at least 98%, at least 99%, at least 99.5%, or 100% identical to SEQ ID NO: 884 or 900 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 95%, at least 96%, at least 97%, at least 98%, at least 99%, at least 99.5%, or 100% identical to SEQ ID NO: 884 or 900 and encodes a multidomain therapeutic protein comprising the sequence set forth in SEQ ID NO: 316. In another example, the nucleic acid construct comprises a sequence at least 99%, at least 99.5%, or 100% identical to SEQ ID NO: 884 or 900. In another example, the nucleic acid construct comprises a sequence at least 99%, at least 99.5%, or 100% identical to SEQ ID NO: 884 or 900 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 99%, at least 99.5%, or 100% identical to SEQ ID NO: 884 or 900 and encodes a multidomain therapeutic protein comprising the sequence set forth in SEQ ID NO: 316. In another example, the nucleic acid construct comprises the sequence set forth in SEQ ID NO: 884 or 900. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the nucleic acid construct in the above examples encodes a multidomain therapeutic protein comprising the sequence set forth in SEQ ID NO: 316. Optionally, the nucleic acid construct in the above examples encodes a multidomain therapeutic protein consisting essentially of the sequence set forth in SEQ ID NO: 316. Optionally, the nucleic acid construct in the above examples encodes a multidomain therapeutic protein consisting of the sequence set forth in SEQ ID NO: 316.

In a specific example of a multidomain therapeutic protein nucleic acid construct, the encoded multidomain therapeutic protein can comprise SEQ ID NO: 316 or can be at least 90%, at least 91%, at least 92%, at least 93%, at least 94%, at least 95%, at least 96%, at least 97%, at least 98%, at least 99%, or at least 99.5% identical to SEQ ID NO: 316. In another specific example, the multidomain therapeutic protein can consist essentially of SEQ ID NO: 316. In another specific example, the multidomain therapeutic protein can consist of SEQ ID NO: 316.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 317-325. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 317-325. In another example, the multidomain therapeutic protein coding sequence is (or comprises a sequence) at least 99%, at least 99.5%, or 100% identical to any one of SEQ ID NOS: 317-325. In another example, the multidomain therapeutic protein coding sequence comprises the sequence set forth in any one of SEQ ID NOS: 317-325. In another example, the multidomain therapeutic protein coding sequence consists essentially of the sequence set forth in any one of SEQ ID NOS: 317-325. In another example, the multidomain therapeutic protein coding sequence consists of the sequence set forth in any one of SEQ ID NOS: 317-325. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 319. In another example, the multidomain therapeutic protein coding sequence is (or comprises a sequence) at least 95%, at least 96%, at least 97%, at least 98%, at least 99%, at least 99.5%, or 100% identical to SEQ ID NO: 319. In another example, the multidomain therapeutic protein coding sequence is (or comprises a sequence) at least 99%, at least 99.5%, or 100% identical to SEQ ID NO: 319. In another example, the multidomain therapeutic protein coding sequence comprises the sequence set forth in SEQ ID NO: 319. In another example, the multidomain therapeutic protein coding sequence consists essentially of the sequence set forth in SEQ ID NO: 319. In another example, the multidomain therapeutic protein coding sequence consists of the sequence set forth in SEQ ID NO: 319. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein comprising the sequence set forth in SEQ ID NO: 316. Optionally, the multidomain therapeutic protein coding sequence in the above examples encodes a multidomain therapeutic protein consisting essentially of the sequence set forth in SEQ ID NO: 316. Optionally, the multidomain therapeutic protein coding sequence in the above examples encodes a multidomain therapeutic protein consisting of the sequence set forth in SEQ ID NO: 316.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 318-325. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 318-325. In another example, the multidomain therapeutic protein coding sequence is (or comprises a sequence) at least 99%, at least 99.5%, or 100% identical to any one of SEQ ID NOS: 318-325. In another example, the multidomain therapeutic protein coding sequence comprises the sequence set forth in any one of SEQ ID NOS: 318-325. In another example, the multidomain therapeutic protein coding sequence consists essentially of the sequence set forth in any one of SEQ ID NOS: 318-325. In another example, the multidomain therapeutic protein coding sequence consists of the sequence set forth in any one of SEQ ID NOS: 318-325. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein comprising the sequence set forth in SEQ ID NO: 316. Optionally, the multidomain therapeutic protein coding sequence in the above examples encodes a multidomain therapeutic protein consisting essentially of the sequence set forth in SEQ ID NO: 316. Optionally, the multidomain therapeutic protein coding sequence in the above examples encodes a multidomain therapeutic protein consisting of the sequence set forth in SEQ ID NO: 316.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 319. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 319 and encodes a multidomain therapeutic protein (or a multidomain therapeutic protein comprising a sequence) at least 99%, at least 99.5%, or 100% identical to SEQ ID NO: 316. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 319 and encodes a multidomain therapeutic protein comprising the sequence set forth in SEQ ID NO: 316. In another example, the multidomain therapeutic protein coding sequence is (or comprises a sequence) at least 95%, at least 96%, at least 97%, at least 98%, at least 99%, at least 99.5%, or 100% identical to SEQ ID NO: 319. In another example, the multidomain therapeutic protein coding sequence is (or comprises a sequence) at least 95%, at least 96%, at least 97%, at least 98%, at least 99%, at least 99.5%, or 100% identical to SEQ ID NO: 319 and encodes a multidomain therapeutic protein (or a multidomain therapeutic protein comprising a sequence) at least 99%, at least 99.5%, or 100% identical to SEQ ID NO: 316. In another example, the multidomain therapeutic protein coding sequence is (or comprises a sequence) at least 95%, at least 96%, at least 97%, at least 98%, at least 99%, at least 99.5%, or 100% identical to SEQ ID NO: 319 and encodes a multidomain therapeutic protein comprising the sequence set forth in SEQ ID NO: 316. In another example, the multidomain therapeutic protein coding sequence is (or comprises a sequence) at least 99%, at least 99.5%, or 100% identical to SEQ ID NO: 319. In another example, the multidomain therapeutic protein coding sequence is (or comprises a sequence) at least 99%, at least 99.5%, or 100% identical to SEQ ID NO: 319 and encodes a multidomain therapeutic protein (or a multidomain therapeutic protein comprising a sequence) at least 99%, at least 99.5%, or 100% identical to SEQ ID NO: 316. In another example, the multidomain therapeutic protein coding sequence is (or comprises a sequence) at least 99%, at least 99.5%, or 100% identical to SEQ ID NO: 319 and encodes a multidomain therapeutic protein comprising the sequence set forth in SEQ ID NO: 316. In another example, the multidomain therapeutic protein coding sequence comprises the sequence set forth in SEQ ID NO: 319. In another example, the multidomain therapeutic protein coding sequence consists essentially of the sequence set forth in SEQ ID NO: 319. In another example, the multidomain therapeutic protein coding sequence consists of the sequence set forth in SEQ ID NO: 319. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein comprising the sequence set forth in SEQ ID NO: 316. Optionally, the multidomain therapeutic protein coding sequence in the above examples encodes a multidomain therapeutic protein consisting essentially of the sequence set forth in SEQ ID NO: 316. Optionally, the multidomain therapeutic protein coding sequence in the above examples encodes a multidomain therapeutic protein consisting of the sequence set forth in SEQ ID NO: 316.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 851. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 851 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 851 and encodes a multidomain therapeutic protein comprising the sequence set forth in SEQ ID NO: 316. In another example, the nucleic acid construct comprises a sequence at least 95%, at least 96%, at least 97%, at least 98%, at least 99%, at least 99.5%, or 100% identical to SEQ ID NO: 851. In another example, the nucleic acid construct comprises a sequence at least 95%, at least 96%, at least 97%, at least 98%, at least 99%, at least 99.5%, or 100% identical to SEQ ID NO: 851 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 95%, at least 96%, at least 97%, at least 98%, at least 99%, at least 99.5%, or 100% identical to SEQ ID NO: 851 and encodes a multidomain therapeutic protein comprising the sequence set forth in SEQ ID NO: 316. In another example, the nucleic acid construct comprises a sequence at least 99%, at least 99.5%, or 100% identical to SEQ ID NO: 851. In another example, the nucleic acid construct comprises a sequence at least 99%, at least 99.5%, or 100% identical to SEQ ID NO: 851 and encodes a multidomain therapeutic protein (or a multidomain therapeutic protein comprising a sequence) at least 99%, at least 99.5%, or 100% identical to SEQ ID NO: 316. In another example, the nucleic acid construct comprises a sequence at least 99%, at least 99.5%, or 100% identical to SEQ ID NO: 851 and encodes a multidomain therapeutic protein comprising the sequence set forth in SEQ ID NO: 316. In another example, the nucleic acid construct comprises the sequence set forth in SEQ ID NO: 851. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the nucleic acid construct in the above examples encodes a multidomain therapeutic protein comprising the sequence set forth in SEQ ID NO: 316. Optionally, the nucleic acid construct in the above examples encodes a multidomain therapeutic protein consisting essentially of the sequence set forth in SEQ ID NO: 316. Optionally, the nucleic acid construct in the above examples encodes a multidomain therapeutic protein consisting of the sequence set forth in SEQ ID NO: 316.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 317. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 317 and encodes a multidomain therapeutic protein (or a multidomain therapeutic protein comprising a sequence) at least 99%, at least 99.5%, or 100% identical to SEQ ID NO: 316. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 317 and encodes a multidomain therapeutic protein comprising the sequence set forth in SEQ ID NO: 316. In another example, the multidomain therapeutic protein coding sequence is (or comprises a sequence) at least 95%, at least 96%, at least 97%, at least 98%, at least 99%, at least 99.5%, or 100% identical to SEQ ID NO: 317. In another example, the multidomain therapeutic protein coding sequence is (or comprises a sequence) at least 95%, at least 96%, at least 97%, at least 98%, at least 99%, at least 99.5%, or 100% identical to SEQ ID NO: 317 and encodes a multidomain therapeutic protein (or a multidomain therapeutic protein comprising a sequence) at least 99%, at least 99.5%, or 100% identical to SEQ ID NO: 316. In another example, the multidomain therapeutic protein coding sequence is (or comprises a sequence) at least 95%, at least 96%, at least 97%, at least 98%, at least 99%, at least 99.5%, or 100% identical to SEQ ID NO: 317 and encodes a multidomain therapeutic protein comprising the sequence set forth in SEQ ID NO: 316. In another example, the multidomain therapeutic protein coding sequence is (or comprises a sequence) at least 99%, at least 99.5%, or 100% identical to SEQ ID NO: 317. In another example, the multidomain therapeutic protein coding sequence is (or comprises a sequence) at least 99%, at least 99.5%, or 100% identical to SEQ ID NO: 317 and encodes a multidomain therapeutic protein (or a multidomain therapeutic protein comprising a sequence) at least 99%, at least 99.5%, or 100% identical to SEQ ID NO: 316. In another example, the multidomain therapeutic protein coding sequence is (or comprises a sequence) at least 99%, at least 99.5%, or 100% identical to SEQ ID NO: 317 and encodes a multidomain therapeutic protein comprising the sequence set forth in SEQ ID NO: 316. In another example, the multidomain therapeutic protein coding sequence comprises the sequence set forth in SEQ ID NO: 317. In another example, the multidomain therapeutic protein coding sequence consists essentially of the sequence set forth in SEQ ID NO: 317. In another example, the multidomain therapeutic protein coding sequence consists of the sequence set forth in SEQ ID NO: 317. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 316 (and, e.g., retaining the activity of native GAA). Optionally, the multidomain therapeutic protein coding sequence in the above examples encodes a multidomain therapeutic protein comprising the sequence set forth in SEQ ID NO: 316. Optionally, the multidomain therapeutic protein coding sequence in the above examples encodes a multidomain therapeutic protein consisting essentially of the sequence set forth in SEQ ID NO: 316. Optionally, the multidomain therapeutic protein coding sequence in the above examples encodes a multidomain therapeutic protein consisting of the sequence set forth in SEQ ID NO: 316.

In some cases, the anti-hTfR:GAA scFv fusion proteins in the format V_{L}-(Gly₄Ser)₃-V_{H}:GAA (Gly₄Ser=SEQ ID NO: 718). In a specific example of a multidomain therapeutic protein nucleic acid construct, the encoded multidomain therapeutic protein can comprise any one of SEQ ID NOS: 688-691 and 793-820 or can be at least 90%, at least 91%, at least 92%, at least 93%, at least 94%, at least 95%, at least 96%, at least 97%, at least 98%, at least 99%, or at least 99.5% identical to any one of SEQ ID NOS: 688-691 and 793-820. In another specific example, the multidomain therapeutic protein can consist essentially of any one of SEQ ID NOS: 688-691 and 793-820. In another specific example, the multidomain therapeutic protein can consist of any one of SEQ ID NOS: 688-691 and 793-820.

In a specific example of a multidomain therapeutic protein nucleic acid construct, the encoded multidomain therapeutic protein can comprise SEQ ID NO: 688 or can be at least 90%, at least 91%, at least 92%, at least 93%, at least 94%, at least 95%, at least 96%, at least 97%, at least 98%, at least 99%, or at least 99.5% identical to SEQ ID NO: 688. In another specific example, the multidomain therapeutic protein can consist essentially of SEQ ID NO: 688. In another specific example, the multidomain therapeutic protein can consist of SEQ ID NO: 688.

In a specific example of a multidomain therapeutic protein nucleic acid construct, the encoded multidomain therapeutic protein can comprise SEQ ID NO: 689 or can be at least 90%, at least 91%, at least 92%, at least 93%, at least 94%, at least 95%, at least 96%, at least 97%, at least 98%, at least 99%, or at least 99.5% identical to SEQ ID NO: 689. In another specific example, the multidomain therapeutic protein can consist essentially of SEQ ID NO: 689. In another specific example, the multidomain therapeutic protein can consist of SEQ ID NO: 689.

In a specific example of a multidomain therapeutic protein nucleic acid construct, the encoded multidomain therapeutic protein can comprise SEQ ID NO: 690 or can be at least 90%, at least 91%, at least 92%, at least 93%, at least 94%, at least 95%, at least 96%, at least 97%, at least 98%, at least 99%, or at least 99.5% identical to SEQ ID NO: 690. In another specific example, the multidomain therapeutic protein can consist essentially of SEQ ID NO: 690. In another specific example, the multidomain therapeutic protein can consist of SEQ ID NO: 690.

In a specific example of a multidomain therapeutic protein nucleic acid construct, the encoded multidomain therapeutic protein can comprise SEQ ID NO: 691 or can be at least 90%, at least 91%, at least 92%, at least 93%, at least 94%, at least 95%, at least 96%, at least 97%, at least 98%, at least 99%, or at least 99.5% identical to SEQ ID NO: 691. In another specific example, the multidomain therapeutic protein can consist essentially of SEQ ID NO: 691. In another specific example, the multidomain therapeutic protein can consist of SEQ ID NO: 691.

In some cases, the anti-hTfR:GAA scFv fusion proteins in the format V_{H}-(Gly₄Ser)₃-V_{L}:GAA (Gly₄Ser=SEQ ID NO: 718). In a specific example of a multidomain therapeutic protein nucleic acid construct, the encoded multidomain therapeutic protein can comprise any one of SEQ ID NOS: 821-824 (optionally lacking the N-terminal MHRPRRRGTRPPPLALLAALLLAARGADA sequence) or can be at least 90%, at least 91%, at least 92%, at least 93%, at least 94%, at least 95%, at least 96%, at least 97%, at least 98%, at least 99%, or at least 99.5% identical to any one of SEQ ID NOS: 821-824 (optionally lacking the N-terminal MHRPRRRGTRPPPLALLAALLLAARGADA sequence). In another specific example, the multidomain therapeutic protein can consist essentially of any one of SEQ ID NOS: 821-824 (optionally lacking the N-terminal MHRPRRRGTRPPPLALLAALLLAARGADA sequence). In another specific example, the multidomain therapeutic protein can consist of any one of SEQ ID NOS: 821-824 (optionally lacking the N-terminal MHRPRRRGTRPPPLALLAALLLAARGADA sequence).

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 692-704. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 692-704. In another example, the multidomain therapeutic protein coding sequence is (or comprises a sequence) at least 99%, at least 99.5%, or 100% identical to any one of SEQ ID NOS: 692-704. In another example, the multidomain therapeutic protein coding sequence comprises the sequence set forth in any one of SEQ ID NOS: 692-704. In another example, the multidomain therapeutic protein coding sequence consists essentially of the sequence set forth in any one of SEQ ID NOS: 692-704. In another example, the multidomain therapeutic protein coding sequence consists of the sequence set forth in any one of SEQ ID NOS: 692-704. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 688-691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to any one of SEQ ID NOS: 688-691 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to any one of SEQ ID NOS: 688-691 (and, e.g., retaining the activity of native GAA). Optionally, the multidomain therapeutic protein coding sequence in the above examples encodes a multidomain therapeutic protein comprising the sequence set forth in any one of SEQ ID NOS: 688-691. Optionally, the multidomain therapeutic protein coding sequence in the above examples encodes a multidomain therapeutic protein consisting essentially of the sequence set forth in any one of SEQ ID NOS: 688-691. Optionally, the multidomain therapeutic protein coding sequence in the above examples encodes a multidomain therapeutic protein consisting of the sequence set forth in any one of SEQ ID NOS: 688-691.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 696-704. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 696-704. In another example, the multidomain therapeutic protein coding sequence is (or comprises a sequence) at least 99%, at least 99.5%, or 100% identical to any one of SEQ ID NOS: 696-704. In another example, the multidomain therapeutic protein coding sequence comprises the sequence set forth in any one of SEQ ID NOS: 696-704. In another example, the multidomain therapeutic protein coding sequence consists essentially of the sequence set forth in any one of SEQ ID NOS: 696-704. In another example, the multidomain therapeutic protein coding sequence consists of the sequence set forth in any one of SEQ ID NOS: 696-704. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 688-691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to any one of SEQ ID NOS: 688-691 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to any one of SEQ ID NOS: 688-691 (and, e.g., retaining the activity of native GAA). Optionally, the multidomain therapeutic protein coding sequence in the above examples encodes a multidomain therapeutic protein comprising the sequence set forth in any one of SEQ ID NOS: 688-691. Optionally, the multidomain therapeutic protein coding sequence in the above examples encodes a multidomain therapeutic protein consisting essentially of the sequence set forth in any one of SEQ ID NOS: 688-691. Optionally, the multidomain therapeutic protein coding sequence in the above examples encodes a multidomain therapeutic protein consisting of the sequence set forth in any one of SEQ ID NOS: 688-691.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 695 and 702-704. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 695 and 702-704. In another example, the multidomain therapeutic protein coding sequence is (or comprises a sequence) at least 99%, at least 99.5%, or 100% identical to any one of SEQ ID NOS: 695 and 702-704. In another example, the multidomain therapeutic protein coding sequence comprises the sequence set forth in any one of SEQ ID NOS: 695 and 702-704. In another example, the multidomain therapeutic protein coding sequence consists essentially of the sequence set forth in any one of SEQ ID NOS: 695 and 702-704. In another example, the multidomain therapeutic protein coding sequence consists of the sequence set forth in any one of SEQ ID NOS: 695 and 702-704. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein comprising the sequence set forth in SEQ ID NO: 691. Optionally, the multidomain therapeutic protein coding sequence in the above examples encodes a multidomain therapeutic protein consisting essentially of the sequence set forth in SEQ ID NO: 691. Optionally, the multidomain therapeutic protein coding sequence in the above examples encodes a multidomain therapeutic protein consisting of the sequence set forth in SEQ ID NO: 691.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 702-704. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 702-704. In another example, the multidomain therapeutic protein coding sequence is (or comprises a sequence) at least 99%, at least 99.5%, or 100% identical to any one of SEQ ID NOS: 702-704. In another example, the multidomain therapeutic protein coding sequence comprises the sequence set forth in any one of SEQ ID NOS: 702-704. In another example, the multidomain therapeutic protein coding sequence consists essentially of the sequence set forth in any one of SEQ ID NOS: 702-704. In another example, the multidomain therapeutic protein coding sequence consists of the sequence set forth in any one of SEQ ID NOS: 702-704. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein comprising the sequence set forth in SEQ ID NO: 691. Optionally, the multidomain therapeutic protein coding sequence in the above examples encodes a multidomain therapeutic protein consisting essentially of the sequence set forth in SEQ ID NO: 691. Optionally, the multidomain therapeutic protein coding sequence in the above examples encodes a multidomain therapeutic protein consisting of the sequence set forth in SEQ ID NO: 691.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 702. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 702 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 702 and encodes a multidomain therapeutic protein comprising the sequence set forth in SEQ ID NO: 691. In another example, the multidomain therapeutic protein coding sequence is (or comprises a sequence) at least 95%, at least 96%, at least 97%, at least 98%, at least 99%, at least 99.5%, or 100% identical to SEQ ID NO: 702. In another example, the multidomain therapeutic protein coding sequence is (or comprises a sequence) at least 95%, at least 96%, at least 97%, at least 98%, at least 99%, at least 99.5%, or 100% identical to SEQ ID NO: 702 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 95%, at least 96%, at least 97%, at least 98%, at least 99%, at least 99.5%, or 100% identical to SEQ ID NO: 702 and encodes a multidomain therapeutic protein comprising the sequence set forth in SEQ ID NO: 691. In another example, the multidomain therapeutic protein coding sequence is (or comprises a sequence) at least 99%, at least 99.5%, or 100% identical to SEQ ID NO: 702. In another example, the multidomain therapeutic protein coding sequence is (or comprises a sequence) at least 99%, at least 99.5%, or 100% identical to SEQ ID NO: 702 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 99%, at least 99.5%, or 100% identical to SEQ ID NO: 702 and encodes a multidomain therapeutic protein comprising the sequence set forth in SEQ ID NO: 691. In another example, the multidomain therapeutic protein coding sequence comprises the sequence set forth in SEQ ID NO: 702. In another example, the multidomain therapeutic protein coding sequence consists essentially of the sequence set forth in SEQ ID NO: 702. In another example, the multidomain therapeutic protein coding sequence consists of the sequence set forth in SEQ ID NO: 702. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein comprising the sequence set forth in SEQ ID NO: 691. Optionally, the multidomain therapeutic protein coding sequence in the above examples encodes a multidomain therapeutic protein consisting essentially of the sequence set forth in SEQ ID NO: 691. Optionally, the multidomain therapeutic protein coding sequence in the above examples encodes a multidomain therapeutic protein consisting of the sequence set forth in SEQ ID NO: 691.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 848. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 848 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 848 and encodes a multidomain therapeutic protein comprising the sequence set forth in SEQ ID NO: 691. In another example, the nucleic acid construct comprises a sequence at least 95%, at least 96%, at least 97%, at least 98%, at least 99%, at least 99.5%, or 100% identical to SEQ ID NO: 848. In another example, the nucleic acid construct comprises a sequence at least 95%, at least 96%, at least 97%, at least 98%, at least 99%, at least 99.5%, or 100% identical to SEQ ID NO: 848 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 95%, at least 96%, at least 97%, at least 98%, at least 99%, at least 99.5%, or 100% identical to SEQ ID NO: 848 and encodes a multidomain therapeutic protein comprising the sequence set forth in SEQ ID NO: 691. In another example, the nucleic acid construct comprises a sequence at least 99%, at least 99.5%, or 100% identical to SEQ ID NO: 848. In another example, the nucleic acid construct comprises a sequence at least 99%, at least 99.5%, or 100% identical to SEQ ID NO: 848 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 99%, at least 99.5%, or 100% identical to SEQ ID NO: 848 and encodes a multidomain therapeutic protein comprising the sequence set forth in SEQ ID NO: 691. In another example, the nucleic acid construct comprises the sequence set forth in SEQ ID NO: 848. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the nucleic acid construct in the above examples encodes a multidomain therapeutic protein comprising the sequence set forth in SEQ ID NO: 691. Optionally, the nucleic acid construct in the above examples encodes a multidomain therapeutic protein consisting essentially of the sequence set forth in SEQ ID NO: 691. Optionally, the nucleic acid construct in the above examples encodes a multidomain therapeutic protein consisting of the sequence set forth in SEQ ID NO: 691.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 703. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 703 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 703 and encodes a multidomain therapeutic protein comprising the sequence set forth in SEQ ID NO: 691. In another example, the multidomain therapeutic protein coding sequence is (or comprises a sequence) at least 95%, at least 96%, at least 97%, at least 98%, at least 99%, at least 99.5%, or 100% identical to SEQ ID NO: 703. In another example, the multidomain therapeutic protein coding sequence is (or comprises a sequence) at least 95%, at least 96%, at least 97%, at least 98%, at least 99%, at least 99.5%, or 100% identical to SEQ ID NO: 703 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 95%, at least 96%, at least 97%, at least 98%, at least 99%, at least 99.5%, or 100% identical to SEQ ID NO: 703 and encodes a multidomain therapeutic protein comprising the sequence set forth in SEQ ID NO: 691. In another example, the multidomain therapeutic protein coding sequence is (or comprises a sequence) at least 99%, at least 99.5%, or 100% identical to SEQ ID NO: 703. In another example, the multidomain therapeutic protein coding sequence is (or comprises a sequence) at least 99%, at least 99.5%, or 100% identical to SEQ ID NO: 703 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 99%, at least 99.5%, or 100% identical to SEQ ID NO: 703 and encodes a multidomain therapeutic protein comprising the sequence set forth in SEQ ID NO: 691. In another example, the multidomain therapeutic protein coding sequence comprises the sequence set forth in SEQ ID NO: 703. In another example, the multidomain therapeutic protein coding sequence consists essentially of the sequence set forth in SEQ ID NO: 703. In another example, the multidomain therapeutic protein coding sequence consists of the sequence set forth in SEQ ID NO: 703. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein comprising the sequence set forth in SEQ ID NO: 691. Optionally, the multidomain therapeutic protein coding sequence in the above examples encodes a multidomain therapeutic protein consisting essentially of the sequence set forth in SEQ ID NO: 691. Optionally, the multidomain therapeutic protein coding sequence in the above examples encodes a multidomain therapeutic protein consisting of the sequence set forth in SEQ ID NO: 691.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 849. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 849 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 849 and encodes a multidomain therapeutic protein comprising the sequence set forth in SEQ ID NO: 691. In another example, the nucleic acid construct comprises a sequence at least 95%, at least 96%, at least 97%, at least 98%, at least 99%, at least 99.5%, or 100% identical to SEQ ID NO: 849. In another example, the nucleic acid construct comprises a sequence at least 95%, at least 96%, at least 97%, at least 98%, at least 99%, at least 99.5%, or 100% identical to SEQ ID NO: 849 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 95%, at least 96%, at least 97%, at least 98%, at least 99%, at least 99.5%, or 100% identical to SEQ ID NO: 849 and encodes a multidomain therapeutic protein comprising the sequence set forth in SEQ ID NO: 691. In another example, the nucleic acid construct comprises a sequence at least 99%, at least 99.5%, or 100% identical to SEQ ID NO: 849. In another example, the nucleic acid construct comprises a sequence at least 99%, at least 99.5%, or 100% identical to SEQ ID NO: 849 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 99%, at least 99.5%, or 100% identical to SEQ ID NO: 849 and encodes a multidomain therapeutic protein comprising the sequence set forth in SEQ ID NO: 691. In another example, the nucleic acid construct comprises the sequence set forth in SEQ ID NO: 849. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the nucleic acid construct in the above examples encodes a multidomain therapeutic protein comprising the sequence set forth in SEQ ID NO: 691. Optionally, the nucleic acid construct in the above examples encodes a multidomain therapeutic protein consisting essentially of the sequence set forth in SEQ ID NO: 691. Optionally, the nucleic acid construct in the above examples encodes a multidomain therapeutic protein consisting of the sequence set forth in SEQ ID NO: 691.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 704. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 704 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 704 and encodes a multidomain therapeutic protein comprising the sequence set forth in SEQ ID NO: 691. In another example, the multidomain therapeutic protein coding sequence is (or comprises a sequence) at least 95%, at least 96%, at least 97%, at least 98%, at least 99%, at least 99.5%, or 100% identical to SEQ ID NO: 704. In another example, the multidomain therapeutic protein coding sequence is (or comprises a sequence) at least 95%, at least 96%, at least 97%, at least 98%, at least 99%, at least 99.5%, or 100% identical to SEQ ID NO: 704 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 95%, at least 96%, at least 97%, at least 98%, at least 99%, at least 99.5%, or 100% identical to SEQ ID NO: 704 and encodes a multidomain therapeutic protein comprising the sequence set forth in SEQ ID NO: 691. In another example, the multidomain therapeutic protein coding sequence is (or comprises a sequence) at least 99%, at least 99.5%, or 100% identical to SEQ ID NO: 704. In another example, the multidomain therapeutic protein coding sequence is (or comprises a sequence) at least 99%, at least 99.5%, or 100% identical to SEQ ID NO: 704 and encodes a multidomain therapeutic protein (or a multidomain therapeutic protein comprising a sequence) at least 99%, at least 99.5%, or 100% identical to SEQ ID NO: 691. In another example, the multidomain therapeutic protein coding sequence is (or comprises a sequence) at least 99%, at least 99.5%, or 100% identical to SEQ ID NO: 704 and encodes a multidomain therapeutic protein comprising the sequence set forth in SEQ ID NO: 691. In another example, the multidomain therapeutic protein coding sequence comprises the sequence set forth in SEQ ID NO: 704. In another example, the multidomain therapeutic protein coding sequence consists essentially of the sequence set forth in SEQ ID NO: 704. In another example, the multidomain therapeutic protein coding sequence consists of the sequence set forth in SEQ ID NO: 704. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the multidomain therapeutic protein coding sequence in the above examples encodes a multidomain therapeutic protein comprising the sequence set forth in SEQ ID NO: 691. Optionally, the multidomain therapeutic protein coding sequence in the above examples encodes a multidomain therapeutic protein consisting essentially of the sequence set forth in SEQ ID NO: 691. Optionally, the multidomain therapeutic protein coding sequence in the above examples encodes a multidomain therapeutic protein consisting of the sequence set forth in SEQ ID NO: 691.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 850. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 850 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 850 and encodes a multidomain therapeutic protein comprising the sequence set forth in SEQ ID NO: 691. In another example, the nucleic acid construct comprises a sequence at least 95%, at least 96%, at least 97%, at least 98%, at least 99%, at least 99.5%, or 100% identical to SEQ ID NO: 850. In another example, nucleic acid construct comprises a sequence at least 95%, at least 96%, at least 97%, at least 98%, at least 99%, at least 99.5%, or 100% identical to SEQ ID NO: 850 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 95%, at least 96%, at least 97%, at least 98%, at least 99%, at least 99.5%, or 100% identical to SEQ ID NO: 850 and encodes a multidomain therapeutic protein comprising the sequence set forth in SEQ ID NO: 691. In another example, the nucleic acid construct comprises a sequence at least 99%, at least 99.5%, or 100% identical to SEQ ID NO: 850. In another example, the nucleic acid construct comprises a sequence at least 99%, at least 99.5%, or 100% identical to SEQ ID NO: 850 and encodes a multidomain therapeutic protein (or a multidomain therapeutic protein comprising a sequence) at least 99%, at least 99.5%, or 100% identical to SEQ ID NO: 691. In another example, the nucleic acid construct comprises a sequence at least 99%, at least 99.5%, or 100% identical to SEQ ID NO: 850 and encodes a multidomain therapeutic protein comprising the sequence set forth in SEQ ID NO: 691. In another example, the nucleic acid construct comprises the sequence set forth in SEQ ID NO: 850. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 691 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 691 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 691 (and, e.g., retaining the activity of native GAA). Optionally, the nucleic acid construct in the above examples encodes a multidomain therapeutic protein comprising the sequence set forth in SEQ ID NO: 691. Optionally, the nucleic acid construct in the above examples encodes a multidomain therapeutic protein consisting essentially of the sequence set forth in SEQ ID NO: 691. Optionally, the nucleic acid construct in the above examples encodes a multidomain therapeutic protein consisting of the sequence set forth in SEQ ID NO: 691.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 694 and 699-701. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 694 and 699-701. In another example, the multidomain therapeutic protein coding sequence is (or comprises a sequence) at least 99%, at least 99.5%, or 100% identical to any one of SEQ ID NOS: 694 and 699-701. In another example, the multidomain therapeutic protein coding sequence comprises the sequence set forth in any one of SEQ ID NOS: 694 and 699-701. In another example, the multidomain therapeutic protein coding sequence consists essentially of the sequence set forth in any one of SEQ ID NOS: 694 and 699-701. In another example, the multidomain therapeutic protein coding sequence consists of the sequence set forth in any one of SEQ ID NOS: 694 and 699-701. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein comprising the sequence set forth in SEQ ID NO: 690. Optionally, the multidomain therapeutic protein coding sequence in the above examples encodes a multidomain therapeutic protein consisting essentially of the sequence set forth in SEQ ID NO: 690. Optionally, the multidomain therapeutic protein coding sequence in the above examples encodes a multidomain therapeutic protein consisting of the sequence set forth in SEQ ID NO: 690.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 699-701. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 699-701. In another example, the multidomain therapeutic protein coding sequence is (or comprises a sequence) at least 99%, at least 99.5%, or 100% identical to any one of SEQ ID NOS: 699-701. In another example, the multidomain therapeutic protein coding sequence comprises the sequence set forth in any one of SEQ ID NOS: 699-701. In another example, the multidomain therapeutic protein coding sequence consists essentially of the sequence set forth in any one of SEQ ID NOS: 699-701. In another example, the multidomain therapeutic protein coding sequence consists of the sequence set forth in any one of SEQ ID NOS: 699-701. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein comprising the sequence set forth in SEQ ID NO: 690. Optionally, the multidomain therapeutic protein coding sequence in the above examples encodes a multidomain therapeutic protein consisting essentially of the sequence set forth in SEQ ID NO: 690. Optionally, the multidomain therapeutic protein coding sequence in the above examples encodes a multidomain therapeutic protein consisting of the sequence set forth in SEQ ID NO: 690.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 699. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 699 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 699 and encodes a multidomain therapeutic protein comprising the sequence set forth in SEQ ID NO: 690. In another example, the multidomain therapeutic protein coding sequence is (or comprises a sequence) at least 95%, at least 96%, at least 97%, at least 98%, at least 99%, at least 99.5%, or 100% identical to SEQ ID NO: 699. In another example, the multidomain therapeutic protein coding sequence is (or comprises a sequence) at least 95%, at least 96%, at least 97%, at least 98%, at least 99%, at least 99.5%, or 100% identical to SEQ ID NO: 699 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 95%, at least 96%, at least 97%, at least 98%, at least 99%, at least 99.5%, or 100% identical to SEQ ID NO: 699 and encodes a multidomain therapeutic protein comprising the sequence set forth in SEQ ID NO: 690. In another example, the multidomain therapeutic protein coding sequence is (or comprises a sequence) at least 99%, at least 99.5%, or 100% identical to SEQ ID NO: 699. In another example, the multidomain therapeutic protein coding sequence is (or comprises a sequence) at least 99%, at least 99.5%, or 100% identical to SEQ ID NO: 699 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 99%, at least 99.5%, or 100% identical to SEQ ID NO: 699 and encodes a multidomain therapeutic protein comprising the sequence set forth in SEQ ID NO: 690. In another example, the multidomain therapeutic protein coding sequence comprises the sequence set forth in SEQ ID NO: 699. In another example, the multidomain therapeutic protein coding sequence consists essentially of the sequence set forth in SEQ ID NO: 699. In another example, the multidomain therapeutic protein coding sequence consists of the sequence set forth in SEQ ID NO: 699. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein comprising the sequence set forth in SEQ ID NO: 690. Optionally, the multidomain therapeutic protein coding sequence in the above examples encodes a multidomain therapeutic protein consisting essentially of the sequence set forth in SEQ ID NO: 690. Optionally, the multidomain therapeutic protein coding sequence in the above examples encodes a multidomain therapeutic protein consisting of the sequence set forth in SEQ ID NO: 690.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 844. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 844 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 844 and encodes a multidomain therapeutic protein comprising the sequence set forth in SEQ ID NO: 690. In another example, the nucleic acid construct comprises a sequence at least 95%, at least 96%, at least 97%, at least 98%, at least 99%, at least 99.5%, or 100% identical to SEQ ID NO: 844. In another example, the nucleic acid construct comprises a sequence at least 95%, at least 96%, at least 97%, at least 98%, at least 99%, at least 99.5%, or 100% identical to SEQ ID NO: 844 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 95%, at least 96%, at least 97%, at least 98%, at least 99%, at least 99.5%, or 100% identical to SEQ ID NO: 844 and encodes a multidomain therapeutic protein comprising the sequence set forth in SEQ ID NO: 690. In another example, the nucleic acid construct comprises a sequence at least 99%, at least 99.5%, or 100% identical to SEQ ID NO: 844. In another example, the nucleic acid construct comprises a sequence at least 99%, at least 99.5%, or 100% identical to SEQ ID NO: 844 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 99%, at least 99.5%, or 100% identical to SEQ ID NO: 844 and encodes a multidomain therapeutic protein comprising the sequence set forth in SEQ ID NO: 690. In another example, the nucleic acid construct comprises the sequence set forth in SEQ ID NO: 844. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the nucleic acid construct in the above examples encodes a multidomain therapeutic protein comprising the sequence set forth in SEQ ID NO: 690. Optionally, the nucleic acid construct in the above examples encodes a multidomain therapeutic protein consisting essentially of the sequence set forth in SEQ ID NO: 690. Optionally, the nucleic acid construct in the above examples encodes a multidomain therapeutic protein consisting of the sequence set forth in SEQ ID NO: 690.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 700. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 700 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 700 and encodes a multidomain therapeutic protein comprising the sequence set forth in SEQ ID NO: 690. In another example, the multidomain therapeutic protein coding sequence is (or comprises a sequence) at least 95%, at least 96%, at least 97%, at least 98%, at least 99%, at least 99.5%, or 100% identical to SEQ ID NO: 700. In another example, the multidomain therapeutic protein coding sequence is (or comprises a sequence) at least 95%, at least 96%, at least 97%, at least 98%, at least 99%, at least 99.5%, or 100% identical to SEQ ID NO: 700 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 95%, at least 96%, at least 97%, at least 98%, at least 99%, at least 99.5%, or 100% identical to SEQ ID NO: 700 and encodes a multidomain therapeutic protein comprising the sequence set forth in SEQ ID NO: 690. In another example, the multidomain therapeutic protein coding sequence is (or comprises a sequence) at least 99%, at least 99.5%, or 100% identical to SEQ ID NO: 700. In another example, the multidomain therapeutic protein coding sequence is (or comprises a sequence) at least 99%, at least 99.5%, or 100% identical to SEQ ID NO: 700 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 99%, at least 99.5%, or 100% identical to SEQ ID NO: 700 and encodes a multidomain therapeutic protein comprising the sequence set forth in SEQ ID NO: 690. In another example, the multidomain therapeutic protein coding sequence comprises the sequence set forth in SEQ ID NO: 700. In another example, the multidomain therapeutic protein coding sequence consists essentially of the sequence set forth in SEQ ID NO: 700. In another example, the multidomain therapeutic protein coding sequence consists of the sequence set forth in SEQ ID NO: 700. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein comprising the sequence set forth in SEQ ID NO: 690. Optionally, the multidomain therapeutic protein coding sequence in the above examples encodes a multidomain therapeutic protein consisting essentially of the sequence set forth in SEQ ID NO: 690. Optionally, the multidomain therapeutic protein coding sequence in the above examples encodes a multidomain therapeutic protein consisting of the sequence set forth in SEQ ID NO: 690.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 845. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 845 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 845 and encodes a multidomain therapeutic protein comprising the sequence set forth in SEQ ID NO: 690. In another example, the nucleic acid construct comprises a sequence at least 95%, at least 96%, at least 97%, at least 98%, at least 99%, at least 99.5%, or 100% identical to SEQ ID NO: 845. In another example, the nucleic acid construct comprises a sequence at least 95%, at least 96%, at least 97%, at least 98%, at least 99%, at least 99.5%, or 100% identical to SEQ ID NO: 845 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 95%, at least 96%, at least 97%, at least 98%, at least 99%, at least 99.5%, or 100% identical to SEQ ID NO: 845 and encodes a multidomain therapeutic protein comprising the sequence set forth in SEQ ID NO: 690. In another example, the nucleic acid construct comprises a sequence at least 99%, at least 99.5%, or 100% identical to SEQ ID NO: 845. In another example, the nucleic acid construct comprises a sequence at least 99%, at least 99.5%, or 100% identical to SEQ ID NO: 845 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 99%, at least 99.5%, or 100% identical to SEQ ID NO: 845 and encodes a multidomain therapeutic protein comprising the sequence set forth in SEQ ID NO: 690. In another example, the nucleic acid construct comprises the sequence set forth in SEQ ID NO: 845. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the nucleic acid construct in the above examples encodes a multidomain therapeutic protein comprising the sequence set forth in SEQ ID NO: 690. Optionally, the nucleic acid construct in the above examples encodes a multidomain therapeutic protein consisting essentially of the sequence set forth in SEQ ID NO: 690. Optionally, the nucleic acid construct in the above examples encodes a multidomain therapeutic protein consisting of the sequence set forth in SEQ ID NO: 690.

In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 701. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 701 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 701 and encodes a multidomain therapeutic protein comprising the sequence set forth in SEQ ID NO: 690. In another example, the multidomain therapeutic protein coding sequence is (or comprises a sequence) at least 95%, at least 96%, at least 97%, at least 98%, at least 99%, at least 99.5%, or 100% identical to SEQ ID NO: 701. In another example, the multidomain therapeutic protein coding sequence is (or comprises a sequence) at least 95%, at least 96%, at least 97%, at least 98%, at least 99%, at least 99.5%, or 100% identical to SEQ ID NO: 701 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 95%, at least 96%, at least 97%, at least 98%, at least 99%, at least 99.5%, or 100% identical to SEQ ID NO: 701 and encodes a multidomain therapeutic protein comprising the sequence set forth in SEQ ID NO: 690. In another example, the multidomain therapeutic protein coding sequence is (or comprises a sequence) at least 99%, at least 99.5%, or 100% identical to SEQ ID NO: 701. In another example, the multidomain therapeutic protein coding sequence is (or comprises a sequence) at least 99%, at least 99.5%, or 100% identical to SEQ ID NO: 701 and encodes a multidomain therapeutic protein (or a multidomain therapeutic protein comprising a sequence) at least 99%, at least 99.5%, or 100% identical to SEQ ID NO: 690. In another example, the multidomain therapeutic protein coding sequence is (or comprises a sequence) at least 99%, at least 99.5%, or 100% identical to SEQ ID NO: 701 and encodes a multidomain therapeutic protein comprising the sequence set forth in SEQ ID NO: 690. In another example, the multidomain therapeutic protein coding sequence comprises the sequence set forth in SEQ ID NO: 701. In another example, the multidomain therapeutic protein coding sequence consists essentially of the sequence set forth in SEQ ID NO: 701. In another example, the multidomain therapeutic protein coding sequence consists of the sequence set forth in SEQ ID NO: 701. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the multidomain therapeutic protein coding sequence in the above examples encodes a multidomain therapeutic protein comprising the sequence set forth in SEQ ID NO: 690. Optionally, the multidomain therapeutic protein coding sequence in the above examples encodes a multidomain therapeutic protein consisting essentially of the sequence set forth in SEQ ID NO: 690. Optionally, the multidomain therapeutic protein coding sequence in the above examples encodes a multidomain therapeutic protein consisting of the sequence set forth in SEQ ID NO: 690.

The nucleic acid construct can comprise, for example, (1) a 5′ ITR (e.g., such as the one set forth in SEQ ID NO: 283), (2) a splice acceptor site (e.g., a mouse Alb exon 2 splice acceptor, such as the one set forth in SEQ ID NO: 286), (3) the multidomain therapeutic protein coding sequence, (4) a polyadenylation signal (e.g., an SV40 polyadenylation signal, such as the one set forth in SEQ ID NO: 827), and (5) a 3′ ITR (e.g., such as the one set forth in SEQ ID NO: 283 or the reverse complement thereof). In one example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 846. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 846 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 90%, at least 91%, at least 92%, at least 93%, at least 94%, at least 95%, at least 96%, at least 97%, at least 98%, at least 99%, at least 99.5%, or 100% identical to SEQ ID NO: 846 and encodes a multidomain therapeutic protein comprising the sequence set forth in SEQ ID NO: 690. In another example, the nucleic acid construct comprises a sequence at least 95%, at least 96%, at least 97%, at least 98%, at least 99%, at least 99.5%, or 100% identical to SEQ ID NO: 846. In another example, the nucleic acid construct comprises a sequence at least 95%, at least 96%, at least 97%, at least 98%, at least 99%, at least 99.5%, or 100% identical to SEQ ID NO: 846 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 95%, at least 96%, at least 97%, at least 98%, at least 99%, at least 99.5%, or 100% identical to SEQ ID NO: 846 and encodes a multidomain therapeutic protein comprising the sequence set forth in SEQ ID NO: 690. In another example, the nucleic acid construct comprises a sequence at least 99%, at least 99.5%, or 100% identical to SEQ ID NO: 846. In another example, the nucleic acid construct comprises a sequence at least 99%, at least 99.5%, or 100% identical to SEQ ID NO: 846 and encodes a multidomain therapeutic protein (or a multidomain therapeutic protein comprising a sequence) at least 99%, at least 99.5%, or 100% identical to SEQ ID NO: 690. In another example, the nucleic acid construct comprises a sequence at least 99%, at least 99.5%, or 100% identical to SEQ ID NO: 846 and encodes a multidomain therapeutic protein comprising the sequence set forth in SEQ ID NO: 690. In another example, the nucleic acid construct comprises the sequence set forth in SEQ ID NO: 846. The multidomain therapeutic protein coding sequence can be, for example, CpG-depleted (e.g., fully CpG-depleted) and/or codon optimized. For example, the multidomain therapeutic protein coding sequence can be CpG depleted (e.g., fully CpG-depleted) and codon optimized. Optionally, the nucleic acid construct encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 690 (and, e.g., retaining the activity of native GAA). Optionally, the nucleic acid construct encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 690 (and, e.g., retaining the activity of native GAA). Optionally, the nucleic acid construct in the above examples encodes a multidomain therapeutic protein (or a multidomain therapeutic protein comprising a sequence) at least 99%, at least 99.5%, or 100% identical to SEQ ID NO: 690 (and, e.g., retaining the activity of native GAA). Optionally, the nucleic acid construct in the above examples encodes a multidomain therapeutic protein comprising the sequence set forth in SEQ ID NO: 690. Optionally, the nucleic acid construct in the above examples encodes a multidomain therapeutic protein consisting essentially of the sequence set forth in SEQ ID NO: 690. Optionally, the nucleic acid construct in the above examples encodes a multidomain therapeutic protein consisting of the sequence set forth in SEQ ID NO: 690.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 692 and 696-698. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 692 and 696-698. In another example, the multidomain therapeutic protein coding sequence is (or comprises a sequence) at least 99%, at least 99.5%, or 100% identical to any one of SEQ ID NOS: 692 and 696-698. In another example, the multidomain therapeutic protein coding sequence comprises the sequence set forth in any one of SEQ ID NOS: 692 and 696-698. In another example, the multidomain therapeutic protein coding sequence consists essentially of the sequence set forth in any one of SEQ ID NOS: 692 and 696-698. In another example, the multidomain therapeutic protein coding sequence consists of the sequence set forth in any one of SEQ ID NOS: 692 and 696-698. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 688 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 688 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 688 (and, e.g., retaining the activity of native GAA). Optionally, the multidomain therapeutic protein coding sequence in the above examples encodes a multidomain therapeutic protein comprising the sequence set forth in SEQ ID NO: 688. Optionally, the multidomain therapeutic protein coding sequence in the above examples encodes a multidomain therapeutic protein consisting essentially of the sequence set forth in SEQ ID NO: 688. Optionally, the multidomain therapeutic protein coding sequence in the above examples encodes a multidomain therapeutic protein consisting of the sequence set forth in SEQ ID NO: 688.

Various codon optimized multidomain therapeutic protein coding sequences are provided. The multidomain therapeutic protein coding sequence can be, for example, CpG-depleted (e.g., fully CpG depleted) and/or codon optimized (e.g., CpG depleted (e.g., fully CpG-depleted) and codon optimized). In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to any one of SEQ ID NOS: 696-698. In another example, the multidomain therapeutic protein coding sequence is (or comprises a sequence) at least 95%, at least 96%, at least 97%, at least 98%, at least 99%, at least 99.5%, or 100% identical to any one of SEQ ID NOS: 696-698. In another example, the multidomain therapeutic protein coding sequence is (or comprises a sequence) at least 99%, at least 99.5%, or 100% identical to any one of SEQ ID NOS: 696-698. In another example, the multidomain therapeutic protein coding sequence comprises the sequence set forth in any one of SEQ ID NOS: 696-698. In another example, the multidomain therapeutic protein coding sequence consists essentially of the sequence set forth in any one of SEQ ID NOS: 696-698. In another example, the multidomain therapeutic protein coding sequence consists of the sequence set forth in any one of SEQ ID NOS: 696-698. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 688 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 688 (and, e.g., retaining the activity of native GAA). Optionally, the GAA coding sequence in the above examples encodes a multidomain therapeutic protein (or a multidomain therapeutic protein comprising a sequence) at least 99%, at least 99.5%, or 100% identical to SEQ ID NO: 688 (and, e.g., retaining the activity of native GAA). Optionally, the multidomain therapeutic protein coding sequence in the above examples encodes a multidomain therapeutic protein comprising the sequence set forth in SEQ ID NO: 688. Optionally, the multidomain therapeutic protein coding sequence in the above examples encodes a multidomain therapeutic protein consisting essentially of the sequence set forth in SEQ ID NO: 688. Optionally, the multidomain therapeutic protein coding sequence in the above examples encodes a multidomain therapeutic protein consisting of the sequence set forth in SEQ ID NO: 688.

Various multidomain therapeutic protein coding sequences are provided. In one example, the multidomain therapeutic protein coding sequence is (or comprises a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 693. In another example, the multidomain therapeutic protein coding sequence is (or comprises a sequence) at least 95%, at least 96%, at least 97%, at least 98%, at least 99%, at least 99.5%, or 100% identical to SEQ ID NO: 693. In another example, the multidomain therapeutic protein coding sequence is (or comprises a sequence) at least 99%, at least 99.5%, or 100% identical to SEQ ID NO: 693. In another example, the multidomain therapeutic protein coding sequence comprises the sequence set forth in SEQ ID NO: 693. In another example, the multidomain therapeutic protein coding sequence consists essentially of the sequence set forth in SEQ ID NO: 693. In another example, the multidomain therapeutic protein coding sequence consists of the sequence set forth in SEQ ID NO: 693. Optionally, the multidomain therapeutic protein coding sequence encodes a multidomain therapeutic protein (or a multidomain therapeutic protein comprising a sequence) at least 90%, at least 91%, at least 92%, at least 93%, at least 94%, at least 95%, at least 96%, at least 97%, at least 98%, at least 99%, at least 99.5%, or 100% identical to SEQ ID NO: 689 (and, e.g., retaining the activity of native GAA). Optionally, the multidomain therapeutic protein coding sequence encodes a multidomain therapeutic protein (or a multidomain therapeutic protein comprising a sequence) at least 95%, at least 96%, at least 97%, at least 98%, at least 99%, at least 99.5%, or 100% identical to SEQ ID NO: 689 (and, e.g., retaining the activity of native GAA). Optionally, the multidomain therapeutic protein coding sequence in the above examples encodes a multidomain therapeutic protein (or a multidomain therapeutic protein comprising a sequence) at least 99%, at least 99.5%, or 100% identical to SEQ ID NO: 689 (and, e.g., retaining the activity of native GAA). Optionally, the multidomain therapeutic protein coding sequence in the above examples encodes a multidomain therapeutic protein comprising the sequence set forth in SEQ ID NO: 689. Optionally, the multidomain therapeutic protein coding sequence in the above examples encodes a multidomain therapeutic protein consisting essentially of the sequence set forth in SEQ ID NO: 689. Optionally, the multidomain therapeutic protein coding sequence in the above examples encodes a multidomain therapeutic protein consisting of the sequence set forth in SEQ ID NO: 689.

When specific multidomain therapeutic protein nucleic acid constructs sequences are disclosed herein, they are meant to encompass the sequence disclosed or the reverse complement of the sequence. For example, if a multidomain therapeutic protein nucleic acid construct disclosed herein consists of the hypothetical sequence 5′-CTGGACCGA-3′, it is also meant to encompass the reverse complement of that sequence (5′-TCGGTCCAG-3′). Likewise, when construct elements are disclosed herein in a specific 5′ to 3′ order, they are also meant to encompass the reverse complement of the order of those elements. One reason for this is that, in many embodiments disclosed herein, the multidomain therapeutic protein nucleic acid constructs are part of a single-stranded recombinant AAV vector. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

### (9) Vectors

The nucleic acid constructs disclosed herein can be provided in a vector for expression or for integration into and expression from a target genomic locus. A vector can comprise additional sequences such as, for example, replication origins, promoters, and genes encoding antibiotic resistance. A vector can also comprise nuclease agent components as disclosed elsewhere herein. For example, a vector can comprise a nucleic acid construct encoding a polypeptide of interest, a CRISPR/Cas system (nucleic acids encoding Cas protein and gRNA), one or more components of a CRISPR/Cas system, or a combination thereof (e.g., a nucleic acid construct and a gRNA). In some cases, a vector comprising a nucleic acid construct encoding a polypeptide of interest does not comprise any components of the nuclease agents described herein (e.g., does not comprise a nucleic acid encoding a Cas protein and does not comprise a nucleic acid encoding a gRNA). Some such vectors comprise homology arms corresponding to target sites in the target genomic locus. Other such vectors do not comprise any homology arms.

Some vectors may be circular. Alternatively, the vector may be linear. The vector can be packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include 10¹², 10¹³, 10¹⁴, 10¹¹, and 10¹⁶ vector genomes/mL. Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹³ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹³, between about 10¹² to about 10¹⁴, between about 10¹² to about 10¹³, between about 10¹³ to about 10¹⁶, between about 10¹⁴ to about 10¹⁶, between about 10¹³ to about 10¹⁶, or between about 10¹³ to about 10¹³ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg. In another example, the viral titer is about 1E12 to about 2E14 vg/kg (e.g., without plasma cell depletion and redosing). In another example, the viral titer is about 3E11 vg/kg to about 5E13 vg/kg (e.g., 2-3× lower with plasma cell depletion, due to 2-3 separate administrations with redosing).

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette can be derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 281, SEQ ID NO: 282, or SEQ ID NO: 283. Other examples of ITRs comprise one or more mutations compared to SEQ ID NO: 281, SEQ ID NO: 282, or SEQ ID NO: 283 and can be at least 90%, at least 91%, at least 92%, at least 93%, at least 94%, at least 95%, at least 96%, at least 97%, at least 98%, or at least 99% identical to SEQ ID NO: 281, SEQ ID NO: 282, or SEQ ID NO: 283. In some rAAV genomes disclosed herein, the nucleic acid construct is flanked on both sides by the same ITR (i.e., the ITR on the 5′ end, and the reverse complement of the ITR on the 3′ end, such as SEQ ID NO: 281 on the 5′ end and SEQ ID NO: 291 on the 3′ end, or SEQ ID NO: 282 on the 5′ end and SEQ ID NO: 825 on the 3′ end, or SEQ ID NO: 283 on the 5′ end and SEQ ID NO: 826 on the 3′ end). In one example, the ITR on each end can comprise, consist essentially of, or consist of SEQ ID NO: 281 (i.e., SEQ ID NO: 281 on the 5′ end, and the reverse complement on the 3′ end). In another example, the ITR on each end can comprise, consist essentially of, or consist of SEQ ID NO: 282 (i.e., SEQ ID NO: 282 on the 5′ end, and the reverse complement on the 3′ end). In one example, the ITR on at least one end comprises, consists essentially of, or consists of SEQ ID NO: 283. In one example, the ITR on the 5′ end comprises, consists essentially of, or consists of SEQ ID NO: 283. In one example, the ITR on the 3′ end comprises, consists essentially of, or consists of SEQ ID NO: 283. In one example, the ITR on each end can comprise, consist essentially of, or consist of SEQ ID NO: 283 (i.e., SEQ ID NO: 283 on the 5′ end, and the reverse complement on the 3′ end). In other rAAV genomes disclosed herein, the nucleic acid construct is flanked by different ITRs on each end. In one example, the ITR on one end comprises, consists essentially of, or consists of SEQ ID NO: 281, and the ITR on the other end comprises, consists essentially of, or consists of SEQ ID NO: 282. In another example, the ITR on one end comprises, consists essentially of, or consists of SEQ ID NO: 281, and the ITR on the other end comprises, consists essentially of, or consists of SEQ ID NO: 283. In one example, the ITR on one end comprises, consists essentially of, or consists of SEQ ID NO: 282, and the ITR on the other end comprises, consists essentially of, or consists of SEQ ID NO: 283.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. An “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8) can be formulated, for example, in 10 mM sodium phosphate, 180 mM sodium chloride, and 0.005% poloxamer 188, at pH 7.3.

### XVI. Nuclease Agents and CRISPR/Cas Systems

The methods and compositions and combinations disclosed herein can utilize nuclease agents such as Clustered Regularly Interspersed Short Palindromic Repeats (CRISPR)/CRISPR-associated (Cas) systems, zinc finger nuclease (ZFN) systems, or Transcription Activator-Like Effector Nuclease (TALEN) systems or components of such systems to modify a target genomic locus in a target gene such as a safe harbor gene (e.g., ALB) for insertion of a nucleic acid construct as disclosed herein. See, e.g., WO 2023/077012 and US 2023-0149563, each of which is herein incorporated by reference in its entirety for all purposes. Generally, the nuclease agents involve the use of engineered cleavage systems to induce a double strand break or a nick (i.e., a single strand break) in a nuclease target site. Cleavage or nicking can occur through the use of specific nucleases such as engineered ZFNs, TALENs, or CRISPR/Cas systems with an engineered guide RNA to guide specific cleavage or nicking of the nuclease target site. Any nuclease agent that induces a nick or double-strand break at a desired target sequence can be used in the methods and compositions disclosed herein. The nuclease agent can be used to create a site of insertion at a desired locus (target gene) within a host genome, at which site the nucleic acid construct is inserted to express the polypeptide of interest. The polypeptide of interest may be exogenous with respect to its insertion site or locus (target gene), such as a safe harbor locus from which polypeptide of interest is not normally expressed. Alternatively, the polypeptide of interest may be non-exogenous with respect to its insertion site, such as insertion into an endogenous locus encoding the polypeptide of interest to correct a defective gene encoding the polypeptide of interest.

In one example, the nuclease agent is a CRISPR/Cas system. In another example, the nuclease agent comprises one or more ZFNs. In yet another example, the nuclease agent comprises one or more TALENs. In a specific example, the CRISPR/Cas systems or components of such systems target an ALB gene or locus (e.g., ALB genomic locus) within a cell, or intron 1 of an ALB gene or locus within a cell. In a more specific example, the CRISPR/Cas systems or components of such systems target a human ALB gene or locus or intron 1 of a human ALB gene or locus within a cell.

CRISPR/Cas systems include transcripts and other elements involved in the expression of, or directing the activity of, Cas genes. A CRISPR/Cas system can be, for example, a type I, a type II, a type III system, or a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed binding or cleavage of nucleic acids. A CRISPR/Cas system targeting an ALB gene or locus comprises a Cas protein (or a nucleic acid encoding the Cas protein) and one or more guide RNAs (or DNAs encoding the one or more guide RNAs), with each of the one or more guide RNAs targeting a different guide RNA target sequence in the target genomic locus (e.g., ALB gene or locus).

CRISPR/Cas systems used in the compositions and methods disclosed herein can be non-naturally occurring. A non-naturally occurring system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### A. Target Genomic Loci and Albumin (ALB)

Any target genomic locus capable of expressing a gene can be used, such as a safe harbor locus (safe harbor gene, such as ALB) or an endogenous locus that would normally encode the polypeptide interest (e.g., a F9 locus for Factor IX, or a GAA locus for lysosomal alpha-glucosidase). The nucleic acid construct can be integrated into any part of the target genomic locus. For example, the nucleic acid construct can be inserted into an intron or an exon of a target genomic locus or can replace one or more introns and/or exons of a target genomic locus. In a specific example, the nucleic acid construct can be integrated into an intron of the target genomic locus, such as the first intron of the target genomic locus (e.g., ALB intron 1). See, e.g., WO 2020/082042, US 2020/0270617, WO 2020/082041, US 2020/0268906, WO 2020/082046, and US 2020/0289628, each of which is herein incorporated by reference in its entirety for all purposes. Constructs integrated into a target genomic locus can be operably linked to an endogenous promoter at the target genomic locus (e.g., the endogenous ALB promoter).

Interactions between integrated exogenous DNA and a host genome can limit the reliability and safety of integration and can lead to overt phenotypic effects that are not due to the targeted genetic modification but are instead due to unintended effects of the integration on surrounding endogenous genes. For example, randomly inserted transgenes can be subject to position effects and silencing, making their expression unreliable and unpredictable. Likewise, integration of exogenous DNA into a chromosomal locus can affect surrounding endogenous genes and chromatin, thereby altering cell behavior and phenotypes. Safe harbor loci include chromosomal loci where transgenes or other exogenous nucleic acid inserts can be stably and reliably expressed in all tissues of interest without overtly altering cell behavior or phenotype (i.e., without any deleterious effects on the host cell). See, e.g., Sadelain et al. (2012) Nat. Rev. Cancer 12:51-58, herein incorporated by reference in its entirety for all purposes. For example, the safe harbor locus can be one in which expression of the inserted gene sequence is not perturbed by any read-through expression from neighboring genes. For example, safe harbor loci can include chromosomal loci where exogenous DNA can integrate and function in a predictable manner without adversely affecting endogenous gene structure or expression. Safe harbor loci can include extragenic regions or intragenic regions such as, for example, loci within genes that are non-essential, dispensable, or able to be disrupted without overt phenotypic consequences.

Such safe harbor loci can offer an open chromatin configuration in all tissues and can be ubiquitously expressed during embryonic development and in adults. See, e.g., Zambrowicz et al. (1997) Proc. Natl. Acad. Sci. U.S.A. 94:3789-3794, herein incorporated by reference in its entirety for all purposes. In addition, the safe harbor loci can be targeted with high efficiency, and safe harbor loci can be disrupted with no overt phenotype. Examples of safe harbor loci include ALB, CCR5, HPRT, AAVS1, and Rosa26. See, e.g., U.S. Pat. Nos. 7,888,121; 7,972,854; 7,914,796; 7,951,925; 8,110,379; 8,409,861; 8,586,526; and US Patent Publication Nos. 2003/0232410; 2005/0208489; 2005/0026157; 2006/0063231; 2008/0159996; 2010/00218264; 2012/0017290; 2011/0265198; 2013/0137104; 2013/0122591; 2013/0177983; 2013/0177960; and 2013/0122591, each of which is herein incorporated by reference in its entirety for all purposes. Other examples of target genomic loci include an ALB locus, a EESYR locus, a SARS locus, position 188,083,272 of human chromosome 1 or its non-human mammalian orthologue, position 3,046,320 of human chromosome 10 or its non-human mammalian orthologue, position 67,328,980 of human chromosome 17 or its non-human mammalian orthologue, an adeno-associated virus site 1 (AAVS1) on chromosome, a naturally occurring site of integration of AAV virus on human chromosome 19 or its non-human mammalian orthologue, a chemokine receptor 5 (CCR5) gene, a chemokine receptor gene encoding an HIV-1 coreceptor, or a mouse Rosa26 locus or its non-murine mammalian orthologue.

In a specific example, a safe harbor locus is a locus within the genome wherein a gene may be inserted without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). The safe harbor locus can allow overexpression of an exogenous gene without significant deleterious effects on the host cell such as a hepatocyte (e.g., without causing apoptosis, necrosis, and/or senescence, or without causing more than 5%, 10%, 15%, 20%, 25%, 30%, or 40% apoptosis, necrosis, and/or senescence as compared to a control population of cells). A desirable safe harbor locus may be one in which expression of the inserted gene sequence is not perturbed by read-through expression from neighboring genes. The safe harbor may be a human safe harbor (e.g., for a liver tissue or hepatocyte host cell).

In a specific example, the target genomic locus is an ALB locus, such as intron 1 of an ALB locus. In a more specific example, the target genomic locus is a human ALB locus, such as intron 1 of a human ALB locus (e.g., SEQ ID NO: 127).

In another specific example, the target genomic locus is a TTR locus, such as intron 1 of a TTR locus. In a more specific example, the target genomic locus is a human TTR locus, such as intron 1 of a human TTR locus.

### B. Cas Proteins

Cas proteins generally comprise at least one RNA recognition or binding domain that can interact with guide RNAs. Cas proteins can also comprise nuclease domains (e.g., DNase domains or RNase domains), DNA-binding domains, helicase domains, protein-protein interaction domains, dimerization domains, and other domains. Some such domains (e.g., DNase domains) can be from a native Cas protein. Other such domains can be added to make a modified Cas protein. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Cscl, Csc2, Csa5, Csn2, Csm2, Csm3, Csm4, Csm5, Csm6, Cmrl, Cmr3, Cmr4, Cmr5, Cmr6, Csbl, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from a Cas9 protein. Cas9 proteins are from a type II CRISPR/Cas system and typically share four key motifs with a conserved architecture. Motifs 1, 2, and 4 are RuvC-like motifs, and motif 3 is an HNH motif. Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (e.g., assigned UniProt accession number Q99ZW2) is an exemplary Cas9 protein. An exemplary SpCas9 protein sequence is set forth in SEQ ID NO: 131 (encoded by the DNA sequence set forth in SEQ ID NO: 132). An exemplary SpCas9 mRNA (cDNA) sequence is set forth in SEQ ID NO: 133. Smaller Cas9 proteins (e.g., Cas9 proteins whose coding sequences are compatible with the maximum AAV packaging capacity when combined with a guide RNA coding sequence and regulatory elements for the Cas9 and guide RNA, such as SaCas9 and CjCas9 and Nme2Cas9) are other exemplary Cas9 proteins. For example, Cas9 from S. aureus (SaCas9) (e.g., assigned UniProt accession number J7RUA5) is another exemplary Cas9 protein. Likewise, Cas9 from Campylobacter jejuni (CjCas9) (e.g., assigned UniProt accession number Q0P897) is another exemplary Cas9 protein. See, e.g., Kim et al. (2017) Nat. Commun. 8:14500, herein incorporated by reference in its entirety for all purposes. SaCas9 is smaller than SpCas9, and CjCas9 is smaller than both SaCas9 and SpCas9. Cas9 from Neisseria meningitidis (Nme2Cas9) is another exemplary Cas9 protein. See, e.g., Edraki et al. (2019) Mol. Cell 73(4):714-726, herein incorporated by reference in its entirety for all purposes. Cas9 proteins from Streptococcus thermophilus (e.g., Streptococcus thermophilus LMD-9 Cas9 encoded by the CRISPR1 locus (St1Cas9) or Streptococcus thermophilus Cas9 from the CRISPR3 locus (St3Cas9)) are other exemplary Cas9 proteins. Cas9 from Francisella novicida (FnCas9) or the RHA Francisella novicida Cas9 variant that recognizes an alternative PAM (E1369R/E1449H/R1556A substitutions) are other exemplary Cas9 proteins. These and other exemplary Cas9 proteins are reviewed, e.g., in Cebrian-Serrano and Davies (2017) Mamm. Genome 28(7):247-261, herein incorporated by reference in its entirety for all purposes. Examples of Cas9 coding sequences, Cas9 mRNAs, and Cas9 protein sequences are provided in WO 2013/176772, WO 2014/065596, WO 2016/106121, WO 2019/067910, WO 2020/082042, US 2020/0270617, WO 2020/082041, US 2020/0268906, WO 2020/082046, and US 2020/0289628, each of which is herein incorporated by reference in its entirety for all purposes. Specific examples of ORFs and Cas9 amino acid sequences are provided in Table 30 at paragraph [0449] WO 2019/067910, and specific examples of Cas9 mRNAs and ORFs are provided in paragraphs [0214]-[0234] of WO 2019/067910. See also WO 2020/082046 A2 (pp. 84-85) and Table 24 in WO 2020/069296, each of which is herein incorporated by reference in its entirety for all purposes. An exemplary SpCas9 protein sequence comprises, consists essentially of, or consists of SEQ ID NO: 134. An exemplary SpCas9 mRNA sequence encoding that SpCas9 protein sequence comprises, consists essentially of, or consists of SEQ ID NO: 135. Another exemplary SpCas9 mRNA sequence encoding that SpCas9 protein sequence comprises, consists essentially of, or consists of SEQ ID NO: 124. Another exemplary SpCas9 mRNA sequence encoding that SpCas9 protein sequence comprises SEQ ID NO: 125. An exemplary SpCas9 coding sequence comprises, consists essentially of, or consists of SEQ ID NO: 126.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2-44 17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number AOQ7Q2) is an exemplary Cpf1 protein.

Another example of a Cas protein is CasX (Cas12e). CasX is an RNA-guided DNA endonuclease that generates a staggered double-strand break in DNA. CasX is less than 1000 amino acids in size. Exemplary CasX proteins are from Deltaproteobacteria (DpbCasX or DpbCas12e) and Planctomycetes (PlmCasX or PlmCas12e). Like Cpf1, CasX uses a single RuvC active site for DNA cleavage. See, e.g., Liu et al. (2019) Nature 566(7743):218-223, herein incorporated by reference in its entirety for all purposes.

Another example of a Cas protein is CasΦ (CasPhi or Cas12j), which is uniquely found in bacteriophages. CasΦ is less than 1000 amino acids in size (e.g., 700-800 amino acids). CasΦ cleavage generates staggered 5′ overhangs. A single RuvC active site in CasΦ is capable of crRNA processing and DNA cutting. See, e.g., Pausch et al. (2020) Science 369(6501):333-337, herein incorporated by reference in its entirety for all purposes.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A. These and other modified Cas proteins are reviewed, e.g., in Cebrian-Serrano and Davies (2017) Mamm. Genome 28(7):247-261, herein incorporated by reference in its entirety for all purposes. Another example of a modified Cas9 protein is xCas9, which is a SpCas9 variant that can recognize an expanded range of PAM sequences. See, e.g., Hu et al. (2018) Nature 556:57-63, herein incorporated by reference in its entirety for all purposes.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of or a property of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Likewise, CasX and CasΦ generally comprise a single RuvC-like domain that cleaves both strands of a target DNA. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337(6096):816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break within a double-stranded target DNA but not a double-strand break (i.e., it can cleave the complementary strand or the non-complementary strand, but not both). If none of the nuclease domains is deleted or mutated in a Cas9 protein, the Cas9 protein will retain double-strand-break-inducing activity. An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Res. 39(21):9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes.

Examples of inactivating mutations in the catalytic domains of xCas9 are the same as those described above for SpCas9. Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) or a substitution at position D10 (e.g., D10A substitution) to generate a Cas nickase. See, e.g., WO 2016/106236, herein incorporated by reference in its entirety for all purposes. Examples of inactivating mutations in the catalytic domains of Nme2Cas9 are also known (e.g., D16A or H588A). Examples of inactivating mutations in the catalytic domains of St1Cas9 are also known (e.g., D9A, D598A, H599A, or N622A). Examples of inactivating mutations in the catalytic domains of St3Cas9 are also known (e.g., D10A or N870A). Examples of inactivating mutations in the catalytic domains of CjCas9 are also known (e.g., combination of D8A or H559A). Examples of inactivating mutations in the catalytic domains of FnCas9 and RHA FnCas9 are also known (e.g., N995A).

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example, one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Examples of inactivating mutations in the catalytic domains of CasX proteins are also known. With reference to CasX proteins from Deltaproteobacteria, D672A, E769A, and D935A (individually or in combination) or corresponding positions in other CasX orthologs are inactivating. See, e.g., Liu et al. (2019) Nature 566(7743):218-223, herein incorporated by reference in its entirety for all purposes.

Examples of inactivating mutations in the catalytic domains of CasΦ proteins are also known. For example, D371A and D394A, alone or in combination, are inactivating mutations. See, e.g., Pausch et al. (2020) Science 369(6501):333-337, herein incorporated by reference in its entirety for all purposes.

Cas proteins can also be operably linked to heterologous polypeptides as fusion proteins. For example, a Cas protein can be fused to a cleavage domain. See WO 2014/089290, herein incorporated by reference in its entirety for all purposes Cas proteins can also be fused to a heterologous polypeptide providing increased or decreased stability. The fused domain or heterologous polypeptide can be located at the N-terminus, the C-terminus, or internally within the Cas protein.

As one example, a Cas protein can be fused to one or more heterologous polypeptides that provide for subcellular localization. Such heterologous polypeptides can include, for example, one or more nuclear localization signals (NLS) such as the monopartite SV40 NLS and/or a bipartite alpha-importin NLS for targeting to the nucleus, a mitochondrial localization signal for targeting to the mitochondria, an ER retention signal, and the like. See, e.g., Lange et al. (2007) J. Biol. Chem. 282(8):5101-5105, herein incorporated by reference in its entirety for all purposes. Such subcellular localization signals can be located at the N-terminus, the C-terminus, or anywhere within the Cas protein. An NLS can comprise a stretch of basic amino acids, and can be a monopartite sequence or a bipartite sequence. Optionally, a Cas protein can comprise two or more NLSs, including an NLS (e.g., an alpha-importin NLS or a monopartite NLS) at the N-terminus and an NLS (e.g., an SV40 NLS or a bipartite NLS) at the C-terminus. A Cas protein can also comprise two or more NLSs at the N-terminus and/or two or more NLSs at the C-terminus.

A Cas protein may, for example, be fused with 1-10 NLSs (e.g., fused with 1-5 NLSs or fused with one NLS. Where one NLS is used, the NLS may be linked at the N-terminus or the C-terminus of the Cas protein sequence. It may also be inserted within the Cas protein sequence. Alternatively, the Cas protein may be fused with more than one NLS. For example, the Cas protein may be fused with 2, 3, 4, or 5 NLSs. In a specific example, the Cas protein may be fused with two NLSs. In certain circumstances, the two NLSs may be the same (e.g., two SV40 NLSs) or different. For example, the Cas protein can be fused to two SV40 NLS sequences linked at the carboxy terminus. Alternatively, the Cas protein may be fused with two NLSs, one linked at the N-terminus and one at the C-terminus. In other examples, the Cas protein may be fused with 3 NLSs or with no NLS. The NLS may be a monopartite sequence, such as, e.g., the SV40 NLS, PKKKRKV (SEQ ID NO: 136) or PKKKRRV (SEQ ID NO: 137). The NLS may be a bipartite sequence, such as the NLS of nucleoplasmin, KRPAATKKAGQAKKKK (SEQ ID NO: 138). In a specific example, a single PKKKRKV (SEQ ID NO: 136) NLS may be linked at the C-terminus of the Cas protein. One or more linkers are optionally included at the fusion site.

Cas proteins can also be operably linked to a cell-penetrating domain or protein transduction domain. For example, the cell-penetrating domain can be derived from the HIV-1 TAT protein, the TLM cell-penetrating motif from human hepatitis B virus, MPG, Pep-1, VP22, a cell penetrating peptide from Herpes simplex virus, or a polyarginine peptide sequence. See, e.g., WO 2014/089290 and WO 2013/176772, each of which is herein incorporated by reference in its entirety for all purposes. The cell-penetrating domain can be located at the N-terminus, the C-terminus, or anywhere within the Cas protein.

Cas proteins can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g., eBFP, eBFP2, Azurite, mKalamal, GFPuv, Sapphire, T-sapphire), cyan fluorescent proteins (e.g., eCFP, Cerulean, CyPet, AmCyanl, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (e.g., mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

Cas proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed. Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. In one example, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. For example, the Cas protein can be tethered to the 5′ end or the 3′ end of the labeled nucleic acid.

Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Optionally, the nucleic acid encoding the Cas protein can be codon optimized for efficient translation into protein in a particular cell or organism. For example, the nucleic acid encoding the Cas protein can be modified to substitute codons having a higher frequency of usage in a bacterial cell, a yeast cell, a human cell, a non-human cell, a mammalian cell, a rodent cell, a mouse cell, a rat cell, or any other host cell of interest, as compared to the naturally occurring polynucleotide sequence. When a nucleic acid encoding the Cas protein is introduced into the cell, the Cas protein can be transiently, conditionally, or constitutively expressed in the cell.

Nucleic acids encoding Cas proteins can be stably integrated in the genome of a cell and operably linked to a promoter active in the cell. Alternatively, nucleic acids encoding Cas proteins can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding a gRNA. Alternatively, it can be in a vector or plasmid that is separate from the vector comprising the DNA encoding the gRNA. Promoters that can be used in an expression construct include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allow for the generation of compact expression cassettes to facilitate delivery. In preferred embodiments, promotors are accepted by regulatory authorities for use in humans. In certain embodiments, promotors drive expression in a liver cell.

Different promoters can be used to drive Cas expression or Cas9 expression. In some methods, small promoters are used so that the Cas or Cas9 coding sequence can fit into an AAV construct. For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., AAV2-mediated delivery, AAV5-mediated delivery, AAV8-mediated delivery, or AAV7m8-mediated delivery). For example, the nuclease agent can be CRISPR/Cas9, and a Cas9 mRNA and a gRNA targeting an intron 1 of an endogenous human ALB locus can be delivered via LNP-mediated delivery or AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

Cas proteins provided as mRNAs can be modified for improved stability and/or immunogenicity properties. The modifications may be made to one or more nucleosides within the mRNA. Examples of chemical modifications to mRNA nucleobases include pseudouridine, 1-methyl-pseudouridine, and 5-methyl-cytidine. mRNA encoding Cas proteins can also be capped. The cap can be, for example, a cap 1 structure in which the +1 ribonucleotide is methylated at the 2′O position of the ribose. The capping can, for example, give superior activity in vivo (e.g., by mimicking a natural cap), can result in a natural structure that reduce stimulation of the innate immune system of the host (e.g., can reduce activation of pattern recognition receptors in the innate immune system). mRNA encoding Cas proteins can also be polyadenylated (to comprise a poly(A) tail). mRNA encoding Cas proteins can also be modified to include pseudouridine (e.g., can be fully substituted with pseudouridine). As another example, capped and polyadenylated Cas mRNA containing N1-methyl-pseudouridine can be used. mRNA encoding Cas proteins can also be modified to include N1-methyl-pseudouridine (e.g., can be fully substituted with N1-methyl-pseudouridine). As another example, Cas mRNA fully substituted with pseudouridine can be used (i.e., all standard uracil residues are replaced with pseudouridine, a uridine isomer in which the uracil is attached with a carbon-carbon bond rather than nitrogen-carbon). As another example, Cas mRNA fully substituted with N1-methyl-pseudouridine can be used (i.e., all standard uracil residues are replaced with N1-methyl-pseudouridine). Likewise, Cas mRNAs can be modified by depletion of uridine using synonymous codons. For example, capped and polyadenylated Cas mRNA fully substituted with pseudouridine can be used. For example, capped and polyadenylated Cas mRNA fully substituted with N1-methyl-pseudouridine can be used.

Cas mRNAs can comprise a modified uridine at least at one, a plurality of, or all uridine positions. The modified uridine can be a uridine modified at the 5 position (e.g., with a halogen, methyl, or ethyl). The modified uridine can be a pseudouridine modified at the 1 position (e.g., with a halogen, methyl, or ethyl). The modified uridine can be, for example, pseudouridine, N1-methyl-pseudouridine, 5-methoxyuridine, 5-iodouridine, or a combination thereof. In some examples, the modified uridine is 5-methoxyuridine. In some examples, the modified uridine is 5-iodouridine. In some examples, the modified uridine is pseudouridine. In some examples, the modified uridine is N1-methyl-pseudouridine. In some examples, the modified uridine is a combination of pseudouridine and N1-methyl-pseudouridine. In some examples, the modified uridine is a combination of pseudouridine and 5-methoxyuridine. In some examples, the modified uridine is a combination of N1-methyl pseudouridine and 5-methoxyuridine. In some examples, the modified uridine is a combination of 5-iodouridine and N1-methyl-pseudouridine. In some examples, the modified uridine is a combination of pseudouridine and 5-iodouridine. In some examples, the modified uridine is a combination of 5-iodouridine and 5-methoxyuridine.

Cas mRNAs disclosed herein can also comprise a 5′ cap, such as a Cap0, Cap1, or Cap2. A 5′ cap is generally a 7-methylguanine ribonucleotide (which may be further modified, e.g., with respect to ARCA) linked through a 5′-triphosphate to the 5′ position of the first nucleotide of the 5′-to-3′ chain of the mRNA (i.e., the first cap-proximal nucleotide). In Cap0, the riboses of the first and second cap-proximal nucleotides of the mRNA both comprise a 2′-hydroxyl. In Cap1, the riboses of the first and second transcribed nucleotides of the mRNA comprise a 2′-methoxy and a 2′-hydroxyl, respectively. In Cap2, the riboses of the first and second cap-proximal nucleotides of the mRNA both comprise a 2′-methoxy. See, e.g., Katibah et al. (2014) Proc. Natl. Acad. Sci. U.S.A. 111(33):12025-30 and Abbas et al. (2017) Proc. Natl. Acad. Sci. U.S.A. 114(11):E2106-E2115, each of which is herein incorporated by reference in its entirety for all purposes. Most endogenous higher eukaryotic mRNAs, including mammalian mRNAs such as human mRNAs, comprise Cap1 or Cap2. Cap0 and other cap structures differing from Cap1 and Cap2 may be immunogenic in mammals, such as humans, due to recognition as non-self by components of the innate immune system such as IFIT-1 and IFIT-5, which can result in elevated cytokine levels including type I interferon. Components of the innate immune system such as IFIT-1 and IFIT-5 may also compete with eIF4E for binding of an mRNA with a cap other than Cap1 or Cap2, potentially inhibiting translation of the mRNA.

A cap can be included co-transcriptionally. For example, ARCA (anti-reverse cap analog; Thermo Fisher Scientific Cat. No. AM8045) is a cap analog comprising a 7-methylguanine 3′-methoxy-5′-triphosphate linked to the 5′ position of a guanine ribonucleotide which can be incorporated in vitro into a transcript at initiation. ARCA results in a Cap0 cap in which the 2′ position of the first cap-proximal nucleotide is hydroxyl. See, e.g., Stepinski et al. (2001) RNA 7:1486-1495, herein incorporated by reference in its entirety for all purposes.

CleanCap™ AG (m7G(5′)ppp(5′)(2′OMeA)pG; TriLink Biotechnologies Cat. No. N-7113) or CleanCap™ GG (m7G(5′)ppp(5′)(2′OMeG)pG; TriLink Biotechnologies Cat. No. N-7133) can be used to provide a Cap1 structure co-transcriptionally. 3′-O-methylated versions of CleanCap™ AG and CleanCap™ GG are also available from TriLink Biotechnologies as Cat. Nos. N-7413 and N-7433, respectively.

Alternatively, a cap can be added to an RNA post-transcriptionally. For example, Vaccinia capping enzyme is commercially available (New England Biolabs Cat. No. M2080S) and has RNA triphosphatase and guanylyltransferase activities, provided by its D1 subunit, and guanine methyltransferase, provided by its D12 subunit. As such, it can add a 7-methylguanine to an RNA, so as to give Cap0, in the presence of S-adenosyl methionine and GTP. See, e.g., Guo and Moss (1990) Proc. Natl. Acad. Sci. U.S.A. 87:4023-4027 and Mao and Shuman (1994) J. Biol. Chem. 269:24472-24479, each of which is herein incorporated by reference in its entirety for all purposes.

Cas mRNAs can further comprise a poly-adenylated (poly-A or poly(A) or poly-adenine) tail. The poly-A tail can, for example, comprise at least 20, at least 30, at least 40, at least 50, at least 60, at least 70, at least 80, at least 90, or at least 100 adenines, and optionally up to 300 adenines. For example, the poly-A tail can comprise 95, 96, 97, 98, 99, or 100 adenine nucleotides.

### C. Guide RNAs

A “guide RNA” or “gRNA” is an RNA molecule that binds to a Cas protein (e.g., Cas9 protein) and targets the Cas protein to a specific location within a target DNA. Guide RNAs can comprise two segments: a “DNA-targeting segment” (also called “guide sequence”) and a “protein-binding segment.” “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA.” See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. A guide RNA can refer to either a CRISPR RNA (crRNA) or the combination of a crRNA and a trans-activating CRISPR RNA (tracrRNA). The crRNA and tracrRNA can be associated as a single RNA molecule (single guide RNA or sgRNA) or in two separate RNA molecules (dual guide RNA or dgRNA). For Cas9, for example, a single-guide RNA can comprise a crRNA fused to a tracrRNA (e.g., via a linker). For Cpf1 and CasΦ, for example, only a crRNA is needed to achieve binding to a target sequence. The terms “guide RNA” and “gRNA” include both double-molecule (i.e., modular) gRNAs and single-molecule gRNAs. In some of the methods and compositions disclosed herein, a gRNA is a S. pyogenes Cas9 gRNA or an equivalent thereof. In some of the methods and compositions disclosed herein, a gRNA is a S. aureus Cas9 gRNA or an equivalent thereof.

An exemplary two-molecule gRNA comprises a crRNA-like (“CRISPR RNA” or “targeter-RNA” or “crRNA” or “crRNA repeat”) molecule and a corresponding tracrRNA-like (“trans-activating CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA. An example of a crRNA tail (e.g., for use with S. pyogenes Cas9), located downstream (3′) of the DNA-targeting segment, comprises, consists essentially of, or consists of GUUUUAGAGCUAUGCU (SEQ ID NO: 139) or GUUUUAGAGCUAUGCUGUUUUG (SEQ ID NO: 140). Any of the DNA-targeting segments disclosed herein can be joined to the 5′ end of SEQ ID NO: 139 or 140 to form a crRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA. Examples of tracrRNA sequences (e.g., for use with S. pyogenes Cas9) comprise, consist essentially of, or consist of any one of AGCAUAGCAAGUUAAAAUAAGGCUAGUCCGUUAUCAACUUGAAAAAGUGGCACC GAGUCGGUGCUUU (SEQ ID NO: 141), AAACAGCAUAGCAAGUUAAAAUAAGGCUAGUCCGUUAUCAACUUGAAAAAGUGG CACCGAGUCGGUGCUUUU (SEQ ID NO: 142), or GUUGGAACCAUUCAAAACAGCAUAGCAAGUUAAAAUAAGGCUAGUCCGUUAUCA ACUUGAAAAAGUGGCACCGAGUCGGUGC (SEQ ID NO: 143).

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to the complementary strand of a target DNA. If used for modification within a cell, the exact sequence of a given crRNA or tracrRNA molecule can be designed to be specific to the species in which the RNA molecules will be used. See, e.g., Mali et al. (2013) Science 339(6121):823-826; Jinek et al. (2012) Science 337(6096):816-821; Hwang et al. (2013) Nat. Biotechnol. 31(3):227-229; Jiang et al. (2013) Nat. Biotechnol. 31(3):233-239; and Cong et al. (2013) Science 339(6121):819-823, each of which is herein incorporated by reference in its entirety for all purposes.

The DNA-targeting segment (crRNA) of a given gRNA comprises a nucleotide sequence that is complementary to a sequence on the complementary strand of the target DNA, as described in more detail below. The DNA-targeting segment of a gRNA interacts with the target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have, for example, a length of at least about 12, at least about 15, at least about 17, at least about 18, at least about 19, at least about 20, at least about 25, at least about 30, at least about 35, or at least about 40 nucleotides. Such DNA-targeting segments can have, for example, a length from about 12 to about 100, from about 12 to about 80, from about 12 to about 50, from about 12 to about 40, from about 12 to about 30, from about 12 to about 25, or from about 12 to about 20 nucleotides. For example, the DNA targeting segment can be from about 15 to about 25 nucleotides (e.g., from about 17 to about 20 nucleotides, or about 17, 18, 19, or 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

In one example, the DNA-targeting segment can be about 20 nucleotides in length. However, shorter and longer sequences can also be used for the targeting segment (e.g., 15-25 nucleotides in length, such as 15, 16, 17, 18, 19, 20, 21, 22, 23, 24, or 25 nucleotides in length). The degree of identity between the DNA-targeting segment and the corresponding guide RNA target sequence (or degree of complementarity between the DNA-targeting segment and the other strand of the guide RNA target sequence) can be, for example, about 75%, about 80%, about 85%, about 90%, about 95%, or 100%. The DNA-targeting segment and the corresponding guide RNA target sequence can contain one or more mismatches. For example, the DNA-targeting segment of the guide RNA and the corresponding guide RNA target sequence can contain 1-4, 1-3, 1-2, 1, 2, 3, or 4 mismatches (e.g., where the total length of the guide RNA target sequence is at least 17, at least 18, at least 19, or at least 20 or more nucleotides). For example, the DNA-targeting segment of the guide RNA and the corresponding guide RNA target sequence can contain 1-4, 1-3, 1-2, 1, 2, 3, or 4 mismatches where the total length of the guide RNA target sequence 20 nucleotides.

As one example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 153-184. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 153-184. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 153-184. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 153-184. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 153-184. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 153-184. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 153-184. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 153-184.

As another example, a guide RNA targeting intron 1 of a human ALB gene can comprise a DNA-targeting segment (i.e., guide sequence) comprising, consisting essentially of, or consisting of the sequence (DNA-targeting segment) set forth in any one of SEQ ID NOS: 159, 153, 156, and 164. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in any one of SEQ ID NOS: 159, 153, 156, and 164. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in any one of SEQ ID NOS: 159, 153, 156, and 164. Alternatively, a guide RNA targeting intron 1 of a human ALB gene can comprise a DNA-targeting segment that is at least 90% or at least 95% identical to the sequence (DNA-targeting segment) set forth in any one of SEQ ID NOS: 159, 153, 156, and 164. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in any one of SEQ ID NOS: 159, 153, 156, and 164. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in any one of SEQ ID NOS: 159, 153, 156, and 164. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in any one of SEQ ID NOS: 159, 153, 156, and 164. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in any one of SEQ ID NOS: 159, 153, 156, and 164.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 159. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 159. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 159. Alternatively, a guide RNA targeting intron 1 of a human ALB gene can comprise a DNA-targeting segment that is at least 90% or at least 95% identical to the sequence (DNA-targeting segment) set forth in SEQ ID NO: 159. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 159. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 159. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 159. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 159.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 153. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 153. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 153. Alternatively, a guide RNA targeting intron 1 of a human ALB gene can comprise a DNA-targeting segment that is at least 90% or at least 95% identical to the sequence (DNA-targeting segment) set forth in SEQ ID NO: 153. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 153. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 153. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 153. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 153.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 156. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 156. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 156. Alternatively, a guide RNA targeting intron 1 of a human ALB gene can comprise a DNA-targeting segment that is at least 90% or at least 95% identical to the sequence (DNA-targeting segment) set forth in SEQ ID NO: 156. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 156. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 156. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 156. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 156.

As another example, a guide RNA targeting intron 1 of a human ALB gene can comprise a DNA-targeting segment (i.e., guide sequence) comprising, consisting essentially of, or consisting of the sequence (DNA-targeting segment) set forth in SEQ ID NO: 164. Alternatively, a guide RNA targeting intron 1 of a human ALB gene can comprise a DNA-targeting segment comprising, consisting essentially of, or consisting of at least 17, at least 18, at least 19, or at least 20 contiguous nucleotides of the sequence (DNA-targeting segment) set forth in SEQ ID NO: 164. Alternatively, a guide RNA targeting intron 1 of a human ALB gene can comprise a DNA-targeting segment that is at least 75%, at least 80%, at least 85%, at least 90%, or at least 95% identical to the sequence (DNA-targeting segment) set forth in SEQ ID NO: 164. Alternatively, a guide RNA targeting intron 1 of a human ALB gene can comprise a DNA-targeting segment that is at least 90% or at least 95% identical to the sequence (DNA-targeting segment) set forth in SEQ ID NO: 164. Alternatively, a guide RNA targeting intron 1 of a human ALB gene can comprise a DNA-targeting segment that is at least 75%, at least 80%, at least 85%, at least 90%, or at least 95% identical to at least 17, at least 18, at least 19, or at least 20 contiguous nucleotides of the sequence (DNA-targeting segment) set forth in SEQ ID NO: 164. Alternatively, a guide RNA targeting intron 1 of a human ALB gene can comprise a DNA-targeting segment that is at least 90% or at least 95% identical to at least 17, at least 18, at least 19, or at least 20 contiguous nucleotides of the sequence (DNA-targeting segment) set forth in SEQ ID NO: 164. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from the sequence (DNA-targeting segment) set forth in SEQ ID NO: 164. Alternatively, a guide RNA targeting intron 1 of a human ALB gene can comprise a DNA-targeting segment comprising, consisting essentially of, or consisting of a sequence that differs by no more than 3, no more than 2, or no more than 1 nucleotide from at least 17, at least 18, at least 19, or at least 20 contiguous nucleotides of the sequence (DNA-targeting segment) set forth in SEQ ID NO: 164.

TABLE 4
Human ALB Intron 1 Guide RNAs.

| Guide | SEQ ID NO (DNA- | SEQ ID NO | SEQ ID NO | SEQ ID NO (Guide RNA |
|---|---|---|---|---|
| RNA | Targeting Segment) | (Unmodified sgRNA) | (Modified sgRNA) | Target Sequence) |
| G009844 | 153 | 185 | 217 | 249 |
| G009851 | 154 | 186 | 218 | 250 |
| G009852 | 155 | 187 | 219 | 251 |
| G009857 | 156 | 188 | 220 | 252 |
| G009858 | 157 | 189 | 221 | 253 |
| G009859 | 158 | 190 | 222 | 254 |
| G009860 | 159 | 191 | 223 | 255 |
| G009861 | 160 | 192 | 224 | 256 |
| G009866 | 161 | 193 | 225 | 257 |
| G009867 | 162 | 194 | 226 | 258 |
| G009868 | 163 | 195 | 227 | 259 |
| G009874 | 164 | 196 | 228 | 260 |
| G012747 | 165 | 197 | 229 | 261 |
| G012748 | 166 | 198 | 230 | 262 |
| G012749 | 167 | 199 | 231 | 263 |
| G012750 | 168 | 200 | 232 | 264 |
| G012751 | 169 | 201 | 233 | 265 |
| G012752 | 170 | 202 | 234 | 266 |
| G012753 | 171 | 203 | 235 | 267 |
| G012754 | 172 | 204 | 236 | 268 |
| G012755 | 173 | 205 | 237 | 269 |
| G012756 | 174 | 206 | 238 | 270 |
| G012757 | 175 | 207 | 239 | 271 |
| G012758 | 176 | 208 | 240 | 272 |
| G012759 | 177 | 209 | 241 | 273 |
| G012760 | 178 | 210 | 242 | 274 |
| G012761 | 179 | 211 | 243 | 275 |
| G012762 | 180 | 212 | 244 | 276 |
| G012763 | 181 | 213 | 245 | 277 |
| G012764 | 182 | 214 | 246 | 278 |
| G012765 | 183 | 215 | 247 | 279 |
| G012766 | 184 | 216 | 248 | 280 |

TABLE 5
Human ALB Intron 1 Guide Sequences.

| Guide Sequence | SEQ ID NO: |
|---|---|
| GAGCAACCUCACUCUUGUCU | 153 |
| AUGCAUUUGUUUCAAAAUAU | 154 |
| UGCAUUUGUUUCAAAAUAUU | 155 |
| AUUUAUGAGAUCAACAGCAC | 156 |
| GAUCAACAGCACAGGUUUUG | 157 |
| UUAAAUAAAGCAUAGUGCAA | 158 |
| UAAAGCAUAGUGCAAUGGAU | 159 |
| UAGUGCAAUGGAUAGGUCUU | 160 |
| UACUAAAACUUUAUUUUACU | 161 |
| AAAGUUGAACAAUAGAAAAA | 162 |
| AAUGCAUAAUCUAAGUCAAA | 163 |
| UAAUAAAAUUCAAACAUCCU | 164 |
| GCAUCUUUAAAGAAUUAUUU | 165 |
| UUUGGCAUUUAUUUCUAAAA | 166 |
| UGUAUUUGUGAAGUCUUACA | 167 |
| UCCUAGGUAAAAAAAAAAAA | 168 |
| UAAUUUUCUUUUGCGCACUA | 169 |
| UGACUGAAACUUCACAGAAU | 170 |
| GACUGAAACUUCACAGAAUA | 171 |
| UUCAUUUUAGUCUGUCUUCU | 172 |
| AUUAUCUAAGUUUGAAUAUA | 173 |
| AAUUUUUAAAAUAGUAUUCU | 174 |
| UGAAUUAUUCUUCUGUUUAA | 175 |
| AUCAUCCUGAGUUUUUCUGU | 176 |
| UUACUAAAACUUUAUUUUAC | 177 |
| ACCUUUUUUUUUUUUUACCU | 178 |
| AGUGCAAUGGAUAGGUCUUU | 179 |
| UGAUUCCUACAGAAAAACUC | 180 |
| UGGGCAAGGGAAGAAAAAAA | 181 |
| CCUCACUCUUGUCUGGGCAA | 182 |
| ACCUCACUCUUGUCUGGGCA | 183 |
| UGAGCAACCUCACUCUUGUC | 184 |

TABLE 6
Human ALB Intron 1 sgRNA Sequences.

| Full Sequence | Full Sequence Modified |
|---|---|
| GAGCAACCUCACUCUUGUCUGUUUUAG | mG*mA*mG*CAACCUCACUCUUGUCUGUUUUAGAmGmCmUmA |
| AGCUAGAAAUAGCAAGUUAAAAUAAG | mGmAmAmAmUmAmGmCAAGUUAAAAUAAGGCUAGUCCGUUA |
| GCUAGUCCGUUAUCAACUUGAAAAAGU | UCAmAmCmUmUmGmAmAmAmAmAmGmUmGmGmCmAmCmCm |
| GGCACCGAGUCGGUGCUUUU | GmAmGmUmCmGmGmUmGmCmU*mU*mU*mU (SEQ ID NO: 217) |
| (SEQ ID NO: 185) | |
| AUGCAUUUGUUUCAAAAUAUGUUUUAG | mA*mU*mG*CAUUUGUUUCAAAAUAUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 218) |
| (SEQ ID NO: 186) | |
| UGCAUUUGUUUCAAAAUAUUGUUUUAG | mUmG*mC*AUUUGUUUCAAAAUAUUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 219) |
| (SEQ ID NO: 187) | |
| AUUUAUGAGAUCAACAGCACGUUUUAG | mA*mU*mU*UAUGAGAUCAACAGCACGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 220) |
| (SEQ ID NO: 188) | |
| GAUCAACAGCACAGGUUUUGGUUUUAG | mG*mA*mU*CAACAGCACAGGUUUUGGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 221) |
| (SEQ ID NO: 189) | |
| UUAAAUAAAGCAUAGUGCAAGUUUUAG | mU*mU*mA*AAUAAAGCAUAGUGCAAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 222) |
| (SEQ ID NO: 190) | |
| UAAAGCAUAGUGCAAUGGAUGUUUUAG | mU*mA*mA*AGCAUAGUGCAAUGGAUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 223) |
| (SEQ ID NO: 191) | |
| UAGUGCAAUGGAUAGGUCUUGUUUUAG | mU*mA*mG*UGCAAUGGAUAGGUCUUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 224) |
| (SEQ ID NO: 192) | |
| UACUAAAACUUUAUUUUACUGUUUUAG | mU*mA*mC*UAAAACUUUAUUUUACUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 225) |
| (SEQ ID NO: 193) | |
| AAAGUUGAACAAUAGAAAAAGUUUUA | mA*mA*mA*GUUGAACAAUAGAAAAAGUUUUAGAmGmCmUmA |
| GAGCUAGAAAUAGCAAGUUAAAAUAAG | mGmAmAmAmUmAmGmCAAGUUAAAAUAAGGCUAGUCCGUUA |
| GCUAGUCCGUUAUCAACUUGAAAAAGU | UCAmAmCmUmUmGmAmAmAmAmAmGmUmGmGmCmAmCmCm |
| GGCACCGAGUCGGUGCUUUU | GmAmGmUmCmGmGmUmGmCmU*mU*mU*mU (SEQ ID NO: 226) |
| (SEQ ID NO: 194) | |
| AAUGCAUAAUCUAAGUCAAAGUUUUAG | mA*mA*mU*GCAUAAUCUAAGUCAAAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 227) |
| (SEQ ID NO: 195) | |
| UAAUAAAAUUCAAACAUCCUGUUUUAG | mU*mA*mA*UAAAAUUCAAACAUCCUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 228) |
| (SEQ ID NO: 196) | |
| GCAUCUUUAAAGAAUUAUUUGUUUUAG | mG*mC*mA*UCUUUAAAGAAUUAUUUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 229) |
| (SEQ ID NO: 197) | |
| UUUGGCAUUUAUUUCUAAAAGUUUUAG | mU*mU*mU*GGCAUUUAUUUCUAAAAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 230) |
| (SEQ ID NO: 198) | |
| UGUAUUUGUGAAGUCUUACAGUUUUAG | mU*mG*mU*AUUUGUGAAGUCUUACAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 231) |
| (SEQ ID NO: 199) | |
| UCCUAGGUAAAAAAAAAAAAGUUUUAG | mU*mC*mC*UAGGUAAAAAAAAAAAAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 232) |
| (SEQ ID NO: 200) | |
| UAAUUUUCUUUUGCGCACUAGUUUUAG | mU*mA*mA*UUUUCUUUUGCGCACUAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 233) |
| (SEQ ID NO: 201) | |
| UGACUGAAACUUCACAGAAUGUUUUAG | mU*mG*mA*CUGAAACUUCACAGAAUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 234) |
| (SEQ ID NO: 202) | |
| GACUGAAACUUCACAGAAUAGUUUUAG | mG*mA*mC*UGAAACUUCACAGAAUAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 235) |
| (SEQ ID NO: 203) | |
| UUCAUUUUAGUCUGUCUUCUGUUUUAG | mU*mU*mC*AUUUUAGUCUGUCUUCUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 236) |
| (SEQ ID NO: 204) | |
| AUUAUCUAAGUUUGAAUAUAGUUUUA | mA*mU*mU*AUCUAAGUUUGAAUAUAGUUUUAGAmGmCmUmA |
| GAGCUAGAAAUAGCAAGUUAAAAUAAG | mGmAmAmAmUmAmGmCAAGUUAAAAUAAGGCUAGUCCGUUA |
| GCUAGUCCGUUAUCAACUUGAAAAAGU | UCAmAmCmUmUmGmAmAmAmAmAmGmUmGmGmCmAmCmCm |
| GGCACCGAGUCGGUGCUUUU | GmAmGmUmCmGmGmUmGmCmU*mU*mU*mU (SEQ ID NO: 237) |
| (SEQ ID NO: 205) | |
| AAUUUUUAAAAUAGUAUUCUGUUUUA | mA*mA*mU*UUUUAAAAUAGUAUUCUGUUUUAGAmGmCmUmA |
| GAGCUAGAAAUAGCAAGUUAAAAUAAG | mGmAmAmAmUmAmGmCAAGUUAAAAUAAGGCUAGUCCGUUA |
| GCUAGUCCGUUAUCAACUUGAAAAAGU | UCAmAmCmUmUmGmAmAmAmAmAmGmUmGmGmCmAmCmCm |
| GGCACCGAGUCGGUGCUUUU | GmAmGmUmCmGmGmUmGmCmU*mU*mU*mU (SEQ ID NO: 238) |
| (SEQ ID NO: 206) | |
| UGAAUUAUUCUUCUGUUUAAGUUUUAG | mU*mG*mA*AUUAUUCUUCUGUUUAAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 239) |
| (SEQ ID NO: 207) | |
| AUCAUCCUGAGUUUUUCUGUGUUUUAG | mA*mU*mC*AUCCUGAGUUUUUCUGUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 240) |
| (SEQ ID NO: 208) | |
| UUACUAAAACUUUAUUUUACGUUUUAG | mU*mU*mA*CUAAAACUUUAUUUUACGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 241) |
| (SEQ ID NO: 209) | |
| ACCUUUUUUUUUUUUUACCUGUUUUAG | mA*mC*mC*UUUUUUUUUUUUUACCUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 242) |
| (SEQ ID NO: 210) | |
| AGUGCAAUGGAUAGGUCUUUGUUUUAG | mA*mG*mU*GCAAUGGAUAGGUCUUUGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 243) |
| (SEQ ID NO: 211) | |
| UGAUUCCUACAGAAAAACUCGUUUUAG | mU*mG*mA*UUCCUACAGAAAAACUCGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 244) |
| (SEQ ID NO: 212) | |
| UGGGCAAGGGAAGAAAAAAAGUUUUA | mU*mG*mG*GCAAGGGAAGAAAAAAAGUUUUAGAmGmCmUmA |
| GAGCUAGAAAUAGCAAGUUAAAAUAAG | mGmAmAmAmUmAmGmCAAGUUAAAAUAAGGCUAGUCCGUUA |
| GCUAGUCCGUUAUCAACUUGAAAAAGU | UCAmAmCmUmUmGmAmAmAmAmAmGmUmGmGmCmAmCmCm |
| GGCACCGAGUCGGUGCUUUU | GmAmGmUmCmGmGmUmGmCmU*mU*mU*mU (SEQ ID NO: 245) |
| (SEQ ID NO: 213) | |
| CCUCACUCUUGUCUGGGCAAGUUUUAG | mC*mC*mU*CACUCUUGUCUGGGCAAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 246) |
| (SEQ ID NO: 214) | |
| ACCUCACUCUUGUCUGGGCAGUUUUAG | mA*mC*mC*UCACUCUUGUCUGGGCAGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 247) |
| (SEQ ID NO: 215) | |
| UGAGCAACCUCACUCUUGUCGUUUUAG | mU*mG*mA*GCAACCUCACUCUUGUCGUUUUAGAmGmCmUmA |
| AGCUAGAAAUAGCAAGUUAAAAUAAGG | mGmAmAmAmUmAmGmCAAGUUAAAAUAAGGCUAGUCCGUUA |
| CUAGUCCGUUAUCAACUUGAAAAAGUG | UCAmAmCmUmUmGmAmAmAmAmAmGmUmGmGmCmAmCmCm |
| GCACCGAGUCGGUGCUUUU | GmAmGmUmCmGmGmUmGmCmU*mU*mU*mU (SEQ ID NO: 248) |
| (SEQ ID NO: 216) | |

TABLE 7
Mouse Alb Intron 1 Guide Sequences.

| Guide Sequence | SEQ ID NO: |
|---|---|
| CACUCUUGUCUGUGGAAACA | 287 |

TABLE 8
Mouse Alb Intron 1 sgRNA Sequences.

| Full Sequence | Full Sequence Modified |
|---|---|
| CACUCUUGUCUGUGGAAACA | mC*mA*mC*UCUUGUCUGUG |
| GUUUUAGAGCUAGAAAUAGC | GAAACAGUUUUAGAmGmCmU |
| AAGUUAAAAUAAGGCUAGUC | mAmGmAmAmAmUmAmGmCAA |
| CGUUAUCAACUUGAAAAAGU | GUUAAAAUAAGGCUAGUCCG |
| GGCACCGAGUCGGUGCUUUU | UUAUCAmAmCmUmUmGmAmA |
| (SEQ ID NO: 289) | mAmAmAmGmUmGmGmCmAmC |
| | mCmGmAmGmUmCmGmGmUmG |
| | mCmU*mU*mU*mU |
| | (SEQ ID NO: 290) |

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise, consist essentially of, or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471(7340):602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting segment of the guide RNA and the complementary strand of the target DNA can be at least 60% (e.g., at least 65%, at least 70%, at least 75%, at least 80%, at least 85%, at least 90%, at least 95%, or 100%). The percent complementarity between the DNA-targeting segment and the complementary strand of the target DNA can be at least 60% over about 20 contiguous nucleotides. As an example, the percent complementarity between the DNA-targeting segment and the complementary strand of the target DNA can be 100% over the 14 contiguous nucleotides at the 5′ end of the complementary strand of the target DNA and as low as 0% over the remainder. In such a case, the DNA-targeting segment can be considered to be 14 nucleotides in length. As another example, the percent complementarity between the DNA-targeting segment and the complementary strand of the target DNA can be 100% over the seven contiguous nucleotides at the 5′ end of the complementary strand of the target DNA and as low as 0% over the remainder. In such a case, the DNA-targeting segment can be considered to be 7 nucleotides in length. In some guide RNAs, at least 17 nucleotides within the DNA-targeting segment are complementary to the complementary strand of the target DNA. For example, the DNA-targeting segment can be 20 nucleotides in length and can comprise 1, 2, or 3 mismatches with the complementary strand of the target DNA. In one example, the mismatches are not adjacent to the region of the complementary strand corresponding to the protospacer adjacent motif (PAM) sequence (i.e., the reverse complement of the PAM sequence) (e.g., the mismatches are in the 5′ end of the DNA-targeting segment of the guide RNA, or the mismatches are at least 2, 3, 4, 5, 6, 7, 8, 9, 10, 11, 12, 13, 14, 15, 16, 17, 18, or 19 base pairs away from the region of the complementary strand corresponding to the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, and the gRNA directs the bound Cas protein to a specific nucleotide sequence within target DNA via the DNA-targeting segment.

Single-guide RNAs can comprise a DNA-targeting segment and a scaffold sequence (i.e., the protein-binding or Cas-binding sequence of the guide RNA). For example, such guide RNAs can have a 5′ DNA-targeting segment joined to a 3′ scaffold sequence. Exemplary scaffold sequences (e.g., for use with S. pyogenes Cas9) comprise, consist essentially of, or consist of:

(version 1; SEQ ID NO: 144)
GUUUUAGAGCUAGAAAUAGCAAGUUAAAAUAAGGCUAGUCCGUUA
UCAACUUGAAAAAGUGGCACCGAGUCGGUGCU;
(version 2; SEQ ID NO: 145)
GUUGGAACCAUUCAAAACAGCAUAGCAAGUUAAAAUAAGGCUAGU
CCGUUAUCAACUUGAAAAAGUGGCACCGAGUCGGUGC;
(version 3; SEQ ID NO: 146)
GUUUUAGAGCUAGAAAUAGCAAGUUAAAAUAAGGCUAGUCCGUUA
UCAACUUGAAAAAGUGGCACCGAGUCGGUGC;
and
(version 4; SEQ ID NO: 147)
GUUUAAGAGCUAUGCUGGAAACAGCAUAGCAAGUUUAAAUAAGGC
UAGUCCGUUAUCAACUUGAAAAAGUGGCACCGAGUCGGUGC;
(version 5; SEQ ID NO: 148)
GUUUUAGAGCUAGAAAUAGCAAGUUAAAAUAAGGCUAGUCCGUUA
UCAACUUGAAAAAGUGGCACCGAGUCGGUGCUUUUUUU;
(version 6; SEQ ID NO: 149)
GUUUUAGAGCUAGAAAUAGCAAGUUAAAAUAAGGCUAGUCCGUUA
UCAACUUGAAAAAGUGGCACCGAGUCGGUGCUUUU;
(version 7; SEQ ID NO: 150)
GUUUAAGAGCUAUGCUGGAAACAGCAUAGCAAGUUUAAAUAAGGC
UAGUCCGUUAUCAACUUGAAAAAGUGGCACCGAGUCGGUGCUUUU
UU;
or
(version 8; SEQ ID NO: 151)
GUUUUAGAGCUAGAAAUAGCAAGUUAAAAUAAGGCUAGUCCGUUA
UCAACUUGGCACCGAGUCGGUGC.

In some guide sgRNAs, the four terminal U residues of version 6 are not present. In some sgRNAs, only 1, 2, or 3 of the four terminal U residues of version 6 are present. Guide RNAs targeting any of the guide RNA target sequences disclosed herein can include, for example, a DNA-targeting segment on the 5′ end of the guide RNA fused to any of the exemplary guide RNA scaffold sequences on the 3′ end of the guide RNA. That is, any of the DNA-targeting segments disclosed herein can be joined to the 5′ end of any one of the above scaffold sequences to form a single guide RNA (chimeric guide RNA).

Guide RNAs can include modifications or sequences that provide for additional desirable features (e.g., modified or regulated stability; subcellular targeting; tracking with a fluorescent label; a binding site for a protein or protein complex; and the like). That is, guide RNAs can include one or more modified nucleosides or nucleotides, or one or more non-naturally and/or naturally occurring components or configurations that are used instead of or in addition to the canonical A, G, C, and U residues. Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleotides within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

Guide RNAs can comprise modified nucleosides and modified nucleotides including, for example, one or more of the following: (1) alteration or replacement of one or both of the non-linking phosphate oxygens and/or of one or more of the linking phosphate oxygens in the phosphodiester backbone linkage (an exemplary backbone modification); (2) alteration or replacement of a constituent of the ribose sugar such as alteration or replacement of the 2′ hydroxyl on the ribose sugar (an exemplary sugar modification); (3) replacement (e.g., wholesale replacement) of the phosphate moiety with dephospho linkers (an exemplary backbone modification); (4) modification or replacement of a naturally occurring nucleobase, including with a non-canonical nucleobase (an exemplary base modification); (5) replacement or modification of the ribose-phosphate backbone (an exemplary backbone modification); (6) modification of the 3′ end or 5′ end of the oligonucleotide (e.g., removal, modification or replacement of a terminal phosphate group or conjugation of a moiety, cap, or linker (such 3′ or 5′ cap modifications may comprise a sugar and/or backbone modification); and (7) modification or replacement of the sugar (an exemplary sugar modification). Other possible guide RNA modifications include modifications of or replacement of uracils or poly-uracil tracts. See, e.g., WO 2015/048577 and US 2016/0237455, each of which is herein incorporated by reference in its entirety for all purposes. Similar modifications can be made to Cas-encoding nucleic acids, such as Cas mRNAs. For example, Cas mRNAs can be modified by depletion of uridine using synonymous codons.

Chemical modifications such at hose listed above can be combined to provide modified gRNAs and/or mRNAs comprising residues (nucleosides and nucleotides) that can have two, three, four, or more modifications. For example, a modified residue can have a modified sugar and a modified nucleobase. In one example, every base of a gRNA is modified (e.g., all bases have a modified phosphate group, such as a phosphorothioate group). For example, all or substantially all of the phosphate groups of a gRNA can be replaced with phosphorothioate groups. Alternatively, or additionally, a modified gRNA can comprise at least one modified residue at or near the 5′ end. Alternatively, or additionally, a modified gRNA can comprise at least one modified residue at or near the 3′ end.

Some gRNAs comprise one, two, three or more modified residues. For example, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100% of the positions in a modified gRNA can be modified nucleosides or nucleotides.

Unmodified nucleic acids can be prone to degradation. Exogenous nucleic acids can also induce an innate immune response. Modifications can help introduce stability and reduce immunogenicity. Some gRNAs described herein can contain one or more modified nucleosides or nucleotides to introduce stability toward intracellular or serum-based nucleases. Some modified gRNAs described herein can exhibit a reduced innate immune response when introduced into a population of cells.

The gRNAs disclosed herein can comprise a backbone modification in which the phosphate group of a modified residue can be modified by replacing one or more of the oxygens with a different substituent. The modification can include the wholesale replacement of an unmodified phosphate moiety with a modified phosphate group as described herein. Backbone modifications of the phosphate backbone can also include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (Rp) or the “S” configuration (Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens.

The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

Scaffolds that can mimic nucleic acids can also be constructed wherein the phosphate linker and ribose sugar are replaced by nuclease resistant nucleoside or nucleotide surrogates. Such modifications may comprise backbone and sugar modifications. In some embodiments, the nucleobases can be tethered by a surrogate backbone. Examples can include, without limitation, the morpholino, cyclobutyl, pyrrolidine and peptide nucleic acid (PNA) nucleoside surrogates.

The modified nucleosides and modified nucleotides can include one or more modifications to the sugar group (a sugar modification). For example, the 2′ hydroxyl group (OH) can be modified (e.g., replaced with a number of different oxy or deoxy substituents. Modifications to the 2′ hydroxyl group can enhance the stability of the nucleic acid since the hydroxyl can no longer be deprotonated to form a 2′-alkoxide ion.

Examples of 2′ hydroxyl group modifications can include alkoxy or aryloxy (OR, wherein “R” can be, e.g., alkyl, cycloalkyl, aryl, aralkyl, heteroaryl or a sugar); polyethyleneglycols (PEG), O(CH₂CH₂O)ₙCH₂CH₂OR wherein R can be, e.g., H or optionally substituted alkyl, and n can be an integer from 0 to 20 (e.g., from 0 to 4, from 0 to 8, from 0 to 10, from 0 to 16, from 1 to 4, from 1 to 8, from 1 to 10, from 1 to 16, from 1 to 20, from 2 to 4, from 2 to 8, from 2 to 10, from 2 to 16, from 2 to 20, from 4 to 8, from 4 to 10, from 4 to 16, and from 4 to 20). The 2′ hydroxyl group modification can be 2′-O-Me. Likewise, the 2′ hydroxyl group modification can be a 2′-fluoro modification, which replaces the 2′ hydroxyl group with a fluoride. The 2′ hydroxyl group modification can include locked nucleic acids (LNA) in which the 2′ hydroxyl can be connected, e.g., by a C₁₋₆ alkylene or C₁₋₆ heteroalkylene bridge, to the 4′ carbon of the same ribose sugar, where exemplary bridges can include methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy, O(CH₂)ₙ-amino, (wherein amino can be, e.g., NH₂; alkylamino, dialkylamino, heterocyclyl, arylamino, diarylamino, heteroarylamino, or diheteroarylamino, ethylenediamine, or polyamino). The 2′ hydroxyl group modification can include unlocked nucleic acids (UNA) in which the ribose ring lacks the C2′-C3′ bond. The 2′ hydroxyl group modification can include the methoxyethyl group (MOE), (OCH₂CH₂OCH₃, e.g., a PEG derivative).

Deoxy 2′ modifications can include hydrogen (i.e. deoxyribose sugars, e.g., at the overhang portions of partially dsRNA); halo (e.g., bromo, chloro, fluoro, or iodo); amino (wherein amino can be, e.g., NH2; alkylamino, dialkylamino, heterocyclyl, arylamino, diarylamino, heteroarylamino, diheteroarylamino, or amino acid); NH(CH₂CH₂NH)ₙCH₂CH₂-amino (wherein amino can be, e.g., as described herein), —NHC(O)R (wherein R can be, e.g., alkyl, cycloalkyl, aryl, aralkyl, heteroaryl or sugar), cyano; mercapto; alkyl-thio-alkyl; thioalkoxy; and alkyl, cycloalkyl, aryl, alkenyl and alkynyl, which may be optionally substituted with e.g., an amino as described herein.

The sugar modification can comprise a sugar group which may also contain one or more carbons that possess the opposite stereochemical configuration than that of the corresponding carbon in ribose. Thus, a modified nucleic acid can include nucleotides containing e.g., arabinose, as the sugar. The modified nucleic acids can also include abasic sugars. These abasic sugars can also be further modified at one or more of the constituent sugar atoms. The modified nucleic acids can also include one or more sugars that are in the L form (e.g., L-nucleosides).

The modified nucleosides and modified nucleotides described herein, which can be incorporated into a modified nucleic acid, can include a modified base, also called a nucleobase. Examples of nucleobases include, but are not limited to, adenine (A), guanine (G), cytosine (C), and uracil (U). These nucleobases can be modified or wholly replaced to provide modified residues that can be incorporated into modified nucleic acids. The nucleobase of the nucleotide can be independently selected from a purine, a pyrimidine, a purine analog, or pyrimidine analog. In some embodiments, the nucleobase can include, for example, naturally-occurring and synthetic derivatives of a base.

In a dual guide RNA, each of the crRNA and the tracrRNA can contain modifications. Such modifications may be at one or both ends of the crRNA and/or tracrRNA. In a sgRNA, one or more residues at one or both ends of the sgRNA may be chemically modified, and/or internal nucleosides may be modified, and/or the entire sgRNA may be chemically modified. Some gRNAs comprise a 5′ end modification. Some gRNAs comprise a 3′ end modification.

The guide RNAs disclosed herein can comprise one of the modification patterns disclosed in WO 2018/107028 A1, herein incorporated by reference in its entirety for all purposes. The guide RNAs disclosed herein can also comprise one of the structures/modification patterns disclosed in US 2017/0114334, herein incorporated by reference in its entirety for all purposes. The guide RNAs disclosed herein can also comprise one of the structures/modification patterns disclosed in WO 2017/136794, WO 2017/004279, US 2018/0187186, or US 2019/0048338, each of which is herein incorporated by reference in its entirety for all purposes.

As one example, nucleotides at the 5′ or 3′ end of a guide RNA can include phosphorothioate linkages (e.g., the bases can have a modified phosphate group that is a phosphorothioate group). For example, a guide RNA can include phosphorothioate linkages between the 2, 3, or 4 terminal nucleotides at the 5′ or 3′ end of the guide RNA. As another example, nucleotides at the 5′ and/or 3′ end of a guide RNA can have 2′-O-methyl modifications. For example, a guide RNA can include 2′-O-methyl modifications at the 2, 3, or 4 terminal nucleotides at the 5′ and/or 3′ end of the guide RNA (e.g., the 5′ end). See, e.g., WO 2017/173054 A1 and Finn et al. (2018) Cell Rep. 22(9):2227-2235, each of which is herein incorporated by reference in its entirety for all purposes. Other possible modifications are described in more detail elsewhere herein. In a specific example, a guide RNA includes 2′-O-methyl analogs and 3′ phosphorothioate internucleotide linkages at the first three 5′ and 3′ terminal RNA residues. Such chemical modifications can, for example, provide greater stability and protection from exonucleases to guide RNAs, allowing them to persist within cells for longer than unmodified guide RNAs. Such chemical modifications can also, for example, protect against innate intracellular immune responses that can actively degrade RNA or trigger immune cascades that lead to cell death.

As one example, any of the guide RNAs described herein can comprise at least one modification. In one example, the at least one modification comprises a 2′-O-methyl (2′-O-Me) modified nucleotide, a phosphorothioate (PS) bond between nucleotides, a 2′-fluoro (2′-F) modified nucleotide, or a combination thereof. For example, the at least one modification can comprise a 2′-O-methyl (2′-O-Me) modified nucleotide. Alternatively, or additionally, the at least one modification can comprise a phosphorothioate (PS) bond between nucleotides. Alternatively, or additionally, the at least one modification can comprise a 2′-fluoro (2′-F) modified nucleotide. In one example, a guide RNA described herein comprises one or more 2′-O-methyl (2′-O-Me) modified nucleotides and one or more phosphorothioate (PS) bonds between nucleotides.

The modifications can occur anywhere in the guide RNA. As one example, the guide RNA comprises a modification at one or more of the first five nucleotides at the 5′ end of the guide RNA, the guide RNA comprises a modification at one or more of the last five nucleotides of the 3′ end of the guide RNA, or a combination thereof. For example, the guide RNA can comprise phosphorothioate bonds between the first four nucleotides of the guide RNA, phosphorothioate bonds between the last four nucleotides of the guide RNA, or a combination thereof. Alternatively, or additionally, the guide RNA can comprise 2′-O-Me modified nucleotides at the first three nucleotides at the 5′ end of the guide RNA, can comprise 2′-O-Me modified nucleotides at the last three nucleotides at the 3′ end of the guide RNA, or a combination thereof.

In one example, a modified gRNA can comprise the following sequence: mN*mN*mN*NNNNNNNNNNNNNNNNNGUUUUAGAmGmCmUmAmGmAmAmAmUmA mGmCAAGUUAAAAUAAGGCUAGUCCGUUAUCAmAmCmUmUmGmAmAmAmAmAm GmUmGmGmCmAmCmCmGmAmGmUmCmGmGmUmGmCmU*mU*mU*mU (SEQ ID NO: 152), where “N” may be any natural or non-natural nucleotide. For example, the totality of N residues comprise a human ALB intron 1 DNA-targeting segment as described herein (e.g., the sequence set forth in SEQ ID NO: 152, wherein the N residues are replaced with the DNA-targeting segment of any one of SEQ ID NOS: 153-184, the DNA-targeting segment of any one of SEQ ID NOS: 159, 153, 156, and 164, or the DNA-targeting segment of SEQ ID NO: 159. For example, a modified gRNA can comprise the sequence set forth in any one of SEQ ID NOS: 217-248, the sequence set forth in any one of SEQ ID NOS: 223, 217, 220, and 228, or the sequence set forth in SEQ ID NO: 223 in Table 6. The terms “mA,” “mC,” “mU,” and “mG” denote a nucleotide (A, C, U, and G, respectively) that has been modified with 2′-O-Me. The symbol “*” depicts a phosphorothioate modification. In certain embodiments, A, C, G, U, and N independently denote a ribose sugar, i.e., 2′-OH. In certain embodiments in the context of a modified sequence, A, C, G, U, and N denote a ribose sugar, i.e., 2′-OH. A phosphorothioate linkage or bond refers to a bond where a sulfur is substituted for one nonbridging phosphate oxygen in a phosphodiester linkage, for example, in the bonds between nucleotides bases. When phosphorothioates are used to generate oligonucleotides, the modified oligonucleotides may also be referred to as S-oligos. The terms A*, C*, U*, or G* denote a nucleotide that is linked to the next (e.g., 3′) nucleotide with a phosphorothioate bond. The terms “mA*,” “mC*,” “mU*,” and “mG*” denote a nucleotide (A, C, U, and G, respectively) that has been substituted with 2′-O-Me and that is linked to the next (e.g., 3′) nucleotide with a phosphorothioate bond.

Another chemical modification that has been shown to influence nucleotide sugar rings is halogen substitution. For example, 2′-fluoro (2′-F) substitution on nucleotide sugar rings can increase oligonucleotide binding affinity and nuclease stability. Abasic nucleotides refer to those which lack nitrogenous bases. Inverted bases refer to those with linkages that are inverted from the normal 5′ to 3′ linkage (i.e., either a 5′ to 5′ linkage or a 3′ to 3′ linkage).

An abasic nucleotide can be attached with an inverted linkage. For example, an abasic nucleotide may be attached to the terminal 5′ nucleotide via a 5′ to 5′ linkage, or an abasic nucleotide may be attached to the terminal 3′ nucleotide via a 3′ to 3′ linkage. An inverted abasic nucleotide at either the terminal 5′ or 3′ nucleotide may also be called an inverted abasic end cap.

In one example, one or more of the first three, four, or five nucleotides at the 5′ terminus, and one or more of the last three, four, or five nucleotides at the 3′ terminus are modified. The modification can be, for example, a 2′-O-Me, 2′-F, inverted abasic nucleotide, phosphorothioate bond, or other nucleotide modification well known to increase stability and/or performance.

In another example, the first four nucleotides at the 5′ terminus, and the last four nucleotides at the 3′ terminus can be linked with phosphorothioate bonds.

In another example, the first three nucleotides at the 5′ terminus, and the last three nucleotides at the 3′ terminus can comprise a 2′-O-methyl (2′-O-Me) modified nucleotide. In another example, the first three nucleotides at the 5′ terminus, and the last three nucleotides at the 3′ terminus comprise a 2′-fluoro (2′-F) modified nucleotide. In another example, the first three nucleotides at the 5′ terminus, and the last three nucleotides at the 3′ terminus comprise an inverted abasic nucleotide.

Guide RNAs can be provided in any form. For example, the gRNA can be provided in the form of RNA, either as two molecules (separate crRNA and tracrRNA) or as one molecule (sgRNA), and optionally in the form of a complex with a Cas protein. The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid, such as a nucleic acid encoding a Cas protein. Alternatively, it can be in a vector or a plasmid that is separate from the vector comprising the nucleic acid encoding the Cas protein. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. Specific examples of suitable promoters include an RNA polymerase III promoter, such as a human U6 promoter, a rat U6 polymerase III promoter, or a mouse U6 polymerase III promoter.

Alternatively, gRNAs can be prepared by various other methods. For example, gRNAs can be prepared by in vitro transcription using, for example, T7 RNA polymerase (see, e.g., WO 2014/089290 and WO 2014/065596, each of which is herein incorporated by reference in its entirety for all purposes). Guide RNAs can also be a synthetically produced molecule prepared by chemical synthesis. For example, a guide RNA can be chemically synthesized to include 2′-O-methyl analogs and 3′ phosphorothioate internucleotide linkages at the first three 5′ and 3′ terminal RNA residues.

Guide RNAs (or nucleic acids encoding guide RNAs) can be in compositions comprising one or more guide RNAs (e.g., 1, 2, 3, 4, or more guide RNAs) and a carrier increasing the stability of the guide RNA (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules. Such compositions can further comprise a Cas protein, such as a Cas9 protein, or a nucleic acid encoding a Cas protein.

As one example, a guide RNA targeting intron 1 of a human ALB gene can comprise, consist essentially of, or consist of the sequence set forth in any one of SEQ ID NOS: 185-248. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 185-248. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 185-248. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 185-248.

As another example, a guide RNA targeting intron 1 of a human ALB gene can comprise, consist essentially of, or consist of the sequence set forth in any one of SEQ ID NOS: 191, 223, 185, 217, 188, 220, 196, and 228. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in any one of SEQ ID NOS: 191, 223, 185, 217, 188, 220, 196, and 228. Alternatively, a guide RNA targeting intron 1 of a human ALB gene can comprise, consist essentially of, or consist of a sequence that is at least 90% or at least 95% identical to the DNA-targeting segment set forth in any one of SEQ ID NOS: 191, 223, 185, 217, 188, 220, 196, and 228. Alternatively, a guide RNA targeting intron 1 of a human ALB gene can comprise, consist essentially of, or consist of a sequence that differs by no more than 3, no more than 2, or no more than 1 nucleotide from the sequence set forth in any one of SEQ ID NOS: 191, 223, 185, 217, 188, 220, 196, and 228.

As another example, a guide RNA targeting intron 1 of a human ALB gene can comprise, consist essentially of, or consist of the sequence set forth in SEQ ID NO: 191 or 223. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 191 or 223. Alternatively, a guide RNA targeting intron 1 of a human ALB gene can comprise, consist essentially of, or consist of a sequence that is at least 90% or at least 95% identical to the DNA-targeting segment set forth in SEQ ID NO: 191 or 223. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 191 or 223.

As another example, a guide RNA targeting intron 1 of a human ALB gene can comprise, consist essentially of, or consist of the sequence set forth in SEQ ID NO: 185 or 217. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 185 or 217. Alternatively, a guide RNA targeting intron 1 of a human ALB gene can comprise, consist essentially of, or consist of a sequence that is at least 90% or at least 95% identical to the DNA-targeting segment set forth in SEQ ID NO: 185 or 217. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 185 or 217.

As another example, a guide RNA targeting intron 1 of a human ALB gene can comprise, consist essentially of, or consist of the sequence set forth in SEQ ID NO: 188 or 220. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 188 or 220. Alternatively, a guide RNA targeting intron 1 of a human ALB gene can comprise, consist essentially of, or consist of a sequence that is at least 90% or at least 95% identical to the DNA-targeting segment set forth in SEQ ID NO: 188 or 220. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 188 or 220.

As another example, a guide RNA targeting intron 1 of a human ALB gene can comprise, consist essentially of, or consist of the sequence set forth in SEQ ID NO: 196 or 228. Alternatively, a guide RNA targeting intron 1 of a human ALB gene can comprise, consist essentially of, or consist of a sequence that is at least 75%, at least 80%, at least 85%, at least 90%, or at least 95% identical to the DNA-targeting segment set forth in SEQ ID NO: 196 or 228. Alternatively, a guide RNA targeting intron 1 of a human ALB gene can comprise, consist essentially of, or consist of a sequence that is at least 90% or at least 95% identical to the DNA-targeting segment set forth in SEQ ID NO: 196 or 228. Alternatively, a guide RNA targeting intron 1 of a human ALB gene can comprise, consist essentially of, or consist of a sequence that differs by no more than 3, no more than 2, or no more than 1 nucleotide from the sequence set forth in SEQ ID NO: 196 or 228.

### D. Guide RNA Target Sequences

Target DNAs for guide RNAs include nucleic acid sequences present in a DNA to which a DNA-targeting segment of a gRNA will bind, provided sufficient conditions for binding exist. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the gRNA can be called the “complementary strand,” and the strand of the target DNA that is complementary to the “complementary strand” (and is therefore not complementary to the Cas protein or gRNA) can be called “noncomplementary strand” or “template strand.”

The target DNA includes both the sequence on the complementary strand to which the guide RNA hybridizes and the corresponding sequence on the non-complementary strand (e.g., adjacent to the protospacer adjacent motif (PAM)). The term “guide RNA target sequence” as used herein refers specifically to the sequence on the non-complementary strand corresponding to (i.e., the reverse complement of) the sequence to which the guide RNA hybridizes on the complementary strand. That is, the guide RNA target sequence refers to the sequence on the non-complementary strand adjacent to the PAM (e.g., upstream or 5′ of the PAM in the case of Cas9). A guide RNA target sequence is equivalent to the DNA-targeting segment of a guide RNA, but with thymines instead of uracils. As one example, a guide RNA target sequence for an SpCas9 enzyme can refer to the sequence upstream of the 5′-NGG-3′ PAM on the non-complementary strand. A guide RNA is designed to have complementarity to the complementary strand of a target DNA, where hybridization between the DNA-targeting segment of the guide RNA and the complementary strand of the target DNA promotes the formation of a CRISPR complex. Full complementarity is not necessarily required, provided that there is sufficient complementarity to cause hybridization and promote formation of a CRISPR complex. If a guide RNA is referred to herein as targeting a guide RNA target sequence, what is meant is that the guide RNA hybridizes to the complementary strand sequence of the target DNA that is the reverse complement of the guide RNA target sequence on the non-complementary strand.

A target DNA or guide RNA target sequence can comprise any polynucleotide, and can be located, for example, in the nucleus or cytoplasm of a cell or within an organelle of a cell, such as a mitochondrion or chloroplast. A target DNA or guide RNA target sequence can be any nucleic acid sequence endogenous or exogenous to a cell. The guide RNA target sequence can be a sequence coding a gene product (e.g., a protein) or a non-coding sequence (e.g., a regulatory sequence) or can include both.

Site-specific binding and cleavage of a target DNA by a Cas protein can occur at locations determined by both (i) base-pairing complementarity between the guide RNA and the complementary strand of the target DNA and (ii) a short motif, called the protospacer adjacent motif (PAM), in the non-complementary strand of the target DNA. The PAM can flank the guide RNA target sequence. Optionally, the guide RNA target sequence can be flanked on the 3′ end by the PAM (e.g., for Cas9). Alternatively, the guide RNA target sequence can be flanked on the 5′ end by the PAM (e.g., for Cpf1). For example, the cleavage site of Cas proteins can be about 1 to about 10 or about 2 to about 5 base pairs (e.g., 3 base pairs) upstream or downstream of the PAM sequence (e.g., within the guide RNA target sequence). In the case of SpCas9, the PAM sequence (i.e., on the non-complementary strand) can be 5′-N₁GG-3′, where N₁ is any DNA nucleotide, and where the PAM is immediately 3′ of the guide RNA target sequence on the non-complementary strand of the target DNA. As such, the sequence corresponding to the PAM on the complementary strand (i.e., the reverse complement) would be 5′-CCN₂-3′, where N₂ is any DNA nucleotide and is immediately 5′ of the sequence to which the DNA-targeting segment of the guide RNA hybridizes on the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′. In the case of DpbCasX, the PAM can have the sequence 5′-TTCN-3′. In the case of CasΦ, the PAM can have the sequence 5′-TBN-3′, wherein B is G, T, or C.

An example of a guide RNA target sequence is a 20-nucleotide DNA sequence immediately preceding an NGG motif recognized by an SpCas9 protein. For example, two examples of guide RNA target sequences plus PAMs are GN₁₉NGG (SEQ ID NO: 128) or N₂₀NGG (SEQ ID NO: 129). See, e.g., WO 2014/165825, herein incorporated by reference in its entirety for all purposes. The guanine at the 5′ end can facilitate transcription by RNA polymerase in cells. Other examples of guide RNA target sequences plus PAMs can include two guanine nucleotides at the 5′ end (e.g., GGN₂₀NGG; SEQ ID NO: 130) to facilitate efficient transcription by T7 polymerase in vitro. See, e.g., WO 2014/065596, herein incorporated by reference in its entirety for all purposes. Other guide RNA target sequences plus PAMs can have between 4-22 nucleotides in length of SEQ ID NOS: 128-130, including the 5′ G or GG and the 3′ GG or NGG. Yet other guide RNA target sequences plus PAMs can have between 14 and 20 nucleotides in length of SEQ ID NOS: 128-130.

Formation of a CRISPR complex hybridized to a target DNA can result in cleavage of one or both strands of the target DNA within or near the region corresponding to the guide RNA target sequence (i.e., the guide RNA target sequence on the non-complementary strand of the target DNA and the reverse complement on the complementary strand to which the guide RNA hybridizes). For example, the cleavage site can be within the guide RNA target sequence (e.g., at a defined location relative to the PAM sequence). The “cleavage site” includes the position of a target DNA at which a Cas protein produces a single-strand break or a double-strand break. The cleavage site can be on only one strand (e.g., when a nickase is used) or on both strands of a double-stranded DNA. Cleavage sites can be at the same position on both strands (producing blunt ends; e.g., Cas9)) or can be at different sites on each strand (producing staggered ends (i.e., overhangs); e.g., Cpf1). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA target sequence or cleavage site of the nickase on the first strand is separated from the guide RNA target sequence or cleavage site of the nickase on the second strand by at least 2, 3, 4, 5, 6, 7, 8, 9, 10, 15, 20, 25, 30, 40, 50, 75, 100, 250, 500, or 1,000 base pairs.

The guide RNA target sequence can also be selected to minimize off-target modification or avoid off-target effects (e.g., by avoiding two or fewer mismatches to off-target genomic sequences).

As one example, a guide RNA targeting intron 1 of a human ALB gene can target the guide RNA target sequence set forth in any one of SEQ ID NOS: 249-280. As another example, a guide RNA targeting intron 1 of a human ALB gene can target at least 17, at least 18, at least 19, or at least 20 contiguous nucleotides of the guide RNA target sequence set forth in any one of SEQ ID NOS: 249-280.

As another example, a guide RNA targeting intron 1 of a human ALB gene can target the guide RNA target sequence set forth in any one of SEQ ID NOS: 255, 249, 252, and 260. As another example, a guide RNA targeting intron 1 of a human ALB gene can target at least 17, at least 18, at least 19, or at least 20 contiguous nucleotides of the guide RNA target sequence set forth in any one of SEQ ID NOS: 255, 249, 252, and 260.

As another example, a guide RNA targeting intron 1 of a human ALB gene can target the guide RNA target sequence set forth in SEQ ID NO: 255. As another example, a guide RNA targeting intron 1 of a human ALB gene can target at least 17, at least 18, at least 19, or at least 20 contiguous nucleotides of the guide RNA target sequence set forth in SEQ ID NO: 255.

As another example, a guide RNA targeting intron 1 of a human ALB gene can target the guide RNA target sequence set forth in SEQ ID NO: 249. As another example, a guide RNA targeting intron 1 of a human ALB gene can target at least 17, at least 18, at least 19, or at least 20 contiguous nucleotides of the guide RNA target sequence set forth in SEQ ID NO: 249.

As another example, a guide RNA targeting intron 1 of a human ALB gene can target the guide RNA target sequence set forth in SEQ ID NO: 252. As another example, a guide RNA targeting intron 1 of a human ALB gene can target at least 17, at least 18, at least 19, or at least 20 contiguous nucleotides of the guide RNA target sequence set forth in SEQ ID NO: 252.

As another example, a guide RNA targeting intron 1 of a human ALB gene can target the guide RNA target sequence set forth in SEQ ID NO: 260. As another example, a guide RNA targeting intron 1 of a human ALB gene can target at least 17, at least 18, at least 19, or at least 20 contiguous nucleotides of the guide RNA target sequence set forth in SEQ ID NO: 260.

TABLE 9
Human ALB Intron 1 Guide RNA Target Sequences.

| Guide RNA Target Sequence | SEQ ID NO: |
|---|---|
| GAGCAACCTCACTCTTGTCT | 249 |
| ATGCATTTGTTTCAAAATAT | 250 |
| TGCATTTGTTTCAAAATATT | 251 |
| ATTTATGAGATCAACAGCAC | 252 |
| GATCAACAGCACAGGTTTTG | 253 |
| TTAAATAAAGCATAGTGCAA | 254 |
| TAAAGCATAGTGCAATGGAT | 255 |
| TAGTGCAATGGATAGGTCTT | 256 |
| TACTAAAACTTTATTTTACT | 257 |
| AAAGTTGAACAATAGAAAAA | 258 |
| AATGCATAATCTAAGTCAAA | 259 |
| TAATAAAATTCAAACATCCT | 260 |
| GCATCTTTAAAGAATTATTT | 261 |
| TTTGGCATTTATTTCTAAAA | 262 |
| TGTATTTGTGAAGTCTTACA | 263 |
| TCCTAGGTAAAAAAAAAAAA | 264 |
| TAATTTTCTTTTGCGCACTA | 265 |
| TGACTGAAACTTCACAGAAT | 266 |
| GACTGAAACTTCACAGAATA | 267 |
| TTCATTTTAGTCTGTCTTCT | 268 |
| ATTATCTAAGTTTGAATATA | 269 |
| AATTTTTAAAATAGTATTCT | 270 |
| TGAATTATTCTTCTGTTTAA | 271 |
| ATCATCCTGAGTTTTTCTGT | 272 |
| TTACTAAAACTTTATTTTAC | 273 |
| ACCTTTTTTTTTTTTTACCT | 274 |
| AGTGCAATGGATAGGTCTTT | 275 |
| TGATTCCTACAGAAAAACTC | 276 |
| TGGGCAAGGGAAGAAAAAAA | 277 |
| CCTCACTCTTGTCTGGGCAA | 278 |
| ACCTCACTCTTGTCTGGGCA | 279 |
| TGAGCAACCTCACTCTTGTC | 280 |

TABLE 10
Mouse Alb Intron 1 Guide
RNA Target Sequences.

| Guide RNA Target Sequence | SEQ ID NO: |
|---|---|
| CACTCTTGTCTGTGGAAACA | 288 |

### E. Lipid Nanoparticles Comprising Nuclease Agents

Lipid nanoparticles comprising the nuclease agents (e.g., CRISPR/Cas systems) are also provided. The lipid nanoparticles can alternatively or additionally comprise a nucleic acid construct encoding a polypeptide of interest as disclosed herein. For example, the lipid nanoparticles can comprise a nuclease agent (e.g., CRISPR/Cas system), can comprise a nucleic acid construct encoding a polypeptide of interest, or can comprise both a nuclease agent (e.g., a CRISPR/Cas system) and a nucleic acid construct encoding a polypeptide of interest. Regarding CRISPR/Cas systems, the lipid nanoparticles can comprise the Cas protein in any form (e.g., protein, DNA, or mRNA) and/or can comprise the guide RNA(s) in any form (e.g., DNA or RNA). In one example, the lipid nanoparticles comprise the Cas protein in the form of mRNA (e.g., a modified RNA as described herein) and the guide RNA(s) in the form of RNA (e.g., a modified guide RNA as disclosed herein). As another example, the lipid nanoparticles can comprise the Cas protein in the form of protein and the guide RNA(s) in the form of RNA). In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine or 1,2-distearoyl-sn-glycero-3-phosphocholine (DSPC). In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a nucleic acid construct encoding a polypeptide of interest as described elsewhere herein. In certain LNPs, the cargo can include an mRNA encoding a Cas nuclease, such as Cas9, a guide RNA or a nucleic acid encoding a guide RNA, and a nucleic acid construct encoding a polypeptide of interest. In some LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z, 12Z)-octadeca-9,12-dienoate), cholesterol, DSPC, and PEG2k-DMG.

In some examples, the LNPs comprise cationic lipids. In some examples, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-13-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl]4-(dimethylamino)butanoate or Dlin-MC3-DMA (MC3))).

Some such lipids suitable for use in the LNPs described herein are biodegradable in vivo.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the LNP. Suitable stealth lipids include lipids having a hydrophilic head group linked to a lipid moiety.

The hydrophilic head group of stealth lipids can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMPE), or 1,2-dimyristoyl-rac-glycero-3-methylpolyoxyethylene glycol-2000 (PEG2k-DMG), 1,2-distearoyl-sn-glycero-3-phosphoethanolamine-N-[methoxy(polyethylene glycol)-2000](PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000](PEG2k-DSA). In one particular example, the stealth lipid may be PEG2k-DMG.

In some embodiments, the PEG lipid includes a glycerol group. In some embodiments, the PEG lipid includes a dimyristoylglycerol (DMG) group. In some embodiments, the PEG lipid comprises PEG2k. In some embodiments, the PEG lipid is a PEG-DMG. In some embodiments, the PEG lipid is a PEG2k-DMG. In some embodiments, the PEG lipid is 1,2-dimyristoyl-rac-glycero-3-methoxypolyethylene glycol-2000. In some embodiments, the PEG2k-DMG is 1,2-dimyristoyl-rac-glycero-3-methoxypolyethylene glycol-2000.

The LNPs can comprise different respective molar ratios of the component lipids in the formulation. The mol-% of the CCD lipid may be, for example, from about 30 mol-% to about 60 mol-%, from about 35 mol-% to about 55 mol-%, from about 40 mol-% to about 50 mol-%, from about 42 mol-% to about 47 mol-%, or about 45%. The mol-% of the helper lipid may be, for example, from about 30 mol-% to about 60 mol-%, from about 35 mol-% to about 55 mol-%, from about 40 mol-% to about 50 mol-%, from about 41 mol-% to about 46 mol-%, or about 44 mol-%. The mol-% of the neutral lipid may be, for example, from about 1 mol-% to about 20 mol-%, from about 5 mol-% to about 15 mol-%, from about 7 mol-% to about 12 mol-%, or about 9 mol-%. The mol-% of the stealth lipid may be, for example, from about 1 mol-% to about 10 mol-%, from about 1 mol-% to about 5 mol-%, from about 1 mol-% to about 3 mol-%, about 2 mol-%, or about 1 mol-%.

The LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100, from about 1 to about 50, from about 1 to about 25, from about 1 to about 10, from about 1 to about 7, from about 3 to about 5, from about 4 to about 5, about 4, about 4.5, or about 5. The N/P ratio can also be from about 4 to about 7 or from about 4.5 to about 6. In specific examples, the N/P ratio can be 4.5 or can be 6.

In some LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25, ranging from about 10:1 to about 1:10, ranging from about 5:1 to about 1:5, or about 1:1. Alternatively, the LNP formulation can include a ratio of Cas mRNA to gRNA nucleic acid from about 1:1 to about 1:5, or about 10:1. Alternatively, the LNP formulation can include a ratio of Cas mRNA to gRNA nucleic acid of about 1:10, 25:1, 10:1, 5:1, 3:1, 1:1, 1:3, 1:5, 1:10, or 1:25. Alternatively, the LNP formulation can include a ratio of Cas mRNA to gRNA nucleic acid of from about 2:1 to about 1:2. Alternatively, the LNP formulation can include a ratio of Cas mRNA to gRNA nucleic acid of from about 1:1 to about 1:2. In specific examples, the ratio of Cas mRNA to gRNA can be about 1:1. In specific examples, the ratio of Cas mRNA to gRNA can be about 1:2. In specific examples, the ratio of Cas mRNA to gRNA can be about 2:1.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

In some LNPs, the cargo can comprise a nucleic acid construct encoding a polypeptide of interest and gRNA. The nucleic acid construct and gRNAs can be in different ratios. For example, the LNP formulation can include a ratio of nucleic acid construct to gRNA nucleic acid ranging from about 25:1 to about 1:25, ranging from about 10:1 to about 1:10, ranging from about 5:1 to about 1:5, or about 1:1. Alternatively, the LNP formulation can include a ratio of nucleic acid construct to gRNA nucleic acid from about 1:1 to about 1:5, about 5:1 to about 1:1, about 10:1, or about 1:10. Alternatively, the LNP formulation can include a ratio of nucleic acid construct to gRNA nucleic acid of about 1:10, about 25:1, about 10:1, about 5:1, about 3:1, about 1:1, about 1:3, about 1:5, about 1:10, or about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

### F. Vectors Comprising Nuclease Agents

The nuclease agents disclosed herein (e.g., ZFN, TALEN, or CRISPR/Cas) can be provided in a vector for expression. A vector can comprise additional sequences such as, for example, replication origins, promoters, and genes encoding antibiotic resistance.

Some vectors may be circular. Alternatively, the vector may be linear. The vector can be in the packaged for delivered via a lipid nanoparticle, liposome, non-lipid nanoparticle, or viral capsid. Non-limiting exemplary vectors include plasmids, phagemids, cosmids, artificial chromosomes, minichromosomes, transposons, viral vectors, and expression vectors.

Introduction of nucleic acids can also be accomplished by virus-mediated delivery, such as AAV-mediated delivery or lentivirus-mediated delivery. The vectors can be, for example, viral vectors such as adeno-associated virus (AAV) vectors. The AAV may be any suitable serotype and may be a single-stranded AAV (ssAAV) or a self-complementary AAV (scAAV). Other exemplary viruses/viral vectors include retroviruses, lentiviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹³, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. In another example, the viral titer is about 2E13 vg/mL or vg/kg. In another example, the viral titer is about 1E12 to about 2E14 vg/kg (e.g., without plasma cell depletion and redosing). In another example, the viral titer is about 3E11 vg/kg to about 5E13 vg/kg (e.g., 2-3× lower with plasma cell depletion, due to 2-3 separate administrations with redosing).

Adeno-associated viruses (AAVs) are endemic in multiple species including human and non-human primates (NHPs). At least 12 natural serotypes and hundreds of natural variants have been isolated and characterized to date. See, e.g., Li et al. (2020) Nat. Rev. Genet. 21:255-272, herein incorporated by reference in its entirety for all purposes. AAV particles are naturally composed of a non-enveloped icosahedral protein capsid containing a single-stranded DNA (ssDNA) genome. The DNA genome is flanked by two inverted terminal repeats (ITRs) which serve as the viral origins of replication and packaging signals. The rep gene encodes four proteins required for viral replication and packaging whilst the cap gene encodes the three structural capsid subunits which dictate the AAV serotype, and the Assembly Activating Protein (AAP) which promotes virion assembly in some serotypes.

Recombinant AAV (rAAV) is currently one of the most commonly used viral vectors used in gene therapy to treat human diseases by delivering therapeutic transgenes to target cells in vivo. Indeed, rAAV vectors are composed of icosahedral capsids similar to natural AAVs, but rAAV virions do not encapsidate AAV protein-coding or AAV replicating sequences. These viral vectors are non-replicating. The only viral sequences required in rAAV vectors are the two ITRs, which are needed to guide genome replication and packaging during manufacturing of the rAAV vector. rAAV genomes are devoid of AAV rep and cap genes, rendering them non-replicating in vivo. rAAV vectors are produced by expressing rep and cap genes along with additional viral helper proteins in trans, in combination with the intended transgene cassette flanked by AAV ITRs.

In therapeutic rAAV genomes, a gene expression cassette is placed between ITR sequences. Typically, rAAV genome cassettes comprise of a promoter to drive expression of a therapeutic transgene, followed by polyadenylation sequence. The ITRs flanking a rAAV expression cassette can be derived from AAV2, the first serotype to be isolated and converted into a recombinant viral vector. Since then, most rAAV production methods rely on AAV2 Rep-based packaging systems. See, e.g., Colella et al. (2017) Mol. Ther. Methods Clin. Dev. 8:87-104, herein incorporated by reference in its entirety for all purposes.

Some non-limiting examples of ITRs that can be used include ITRs comprising, consisting essentially of, or consisting of SEQ ID NO: 281, SEQ ID NO: 282, or SEQ ID NO: 283. Other examples of ITRs comprise one or more mutations compared to SEQ ID NO: 281, SEQ ID NO: 282, or SEQ ID NO: 283 and can be at least 90%, at least 91%, at least 92%, at least 93%, at least 94%, at least 95%, at least 96%, at least 97%, at least 98%, or at least 99% identical to SEQ ID NO: 281, SEQ ID NO: 282, or SEQ ID NO: 283. In some rAAV genomes disclosed herein, the nucleic acid encoding the nuclease agent (or component thereof) is flanked on both sides by the same ITR (i.e., the ITR on the 5′ end, and the reverse complement of the ITR on the 3′ end, such as SEQ ID NO: 281 on the 5′ end and SEQ ID NO: 291 on the 3′ end, or SEQ ID NO: 282 on the 5′ end and SEQ ID NO: 825 on the 3′ end, or SEQ ID NO: 283 on the 5′ end and SEQ ID NO: 826 on the 3′ end). In one example, the ITR on each end can comprise, consist essentially of, or consist of SEQ ID NO: 281 (i.e., SEQ ID NO: 281 on the 5′ end, and the reverse complement on the 3′ end). In another example, the ITR on each end can comprise, consist essentially of, or consist of SEQ ID NO: 282 (i.e., SEQ ID NO: 282 on the 5′ end, and the reverse complement on the 3′ end). In one example, the ITR on at least one end comprises, consists essentially of, or consists of SEQ ID NO: 283. In one example, the ITR on the 5′ end comprises, consists essentially of, or consists of SEQ ID NO: 283. In one example, the ITR on the 3′ end comprises, consists essentially of, or consists of SEQ ID NO: 283. In one example, the ITR on each end can comprise, consist essentially of, or consist of SEQ ID NO: 283 (i.e., SEQ ID NO: 283 on the 5′ end, and the reverse complement on the 3′ end). In one example, the ITR on each end can comprise, consist essentially of, or consist of SEQ ID NO: 283. In other rAAV genomes disclosed herein, the nucleic acid encoding the nuclease agent (or component thereof) is flanked by different ITRs on each end. In one example, the ITR on one end comprises, consists essentially of, or consists of SEQ ID NO: 281, and the ITR on the other end comprises, consists essentially of, or consists of SEQ ID NO: 282. In another example, the ITR on one end comprises, consists essentially of, or consists of SEQ ID NO: 281, and the ITR on the other end comprises, consists essentially of, or consists of SEQ ID NO: 283. In one example, the ITR on one end comprises, consists essentially of, or consists of SEQ ID NO: 282, and the ITR on the other end comprises, consists essentially of, or consists of SEQ ID NO: 283.

The specific serotype of a recombinant AAV vector influences its in vivo tropism to specific tissues. AAV capsid proteins are responsible for mediating attachment and entry into target cells, followed by endosomal escape and trafficking to the nucleus. Thus, the choice of serotype when developing a rAAV vector will influence what cell types and tissues the vector is most likely to bind to and transduce when injected in vivo. Several serotypes of rAAVs, including rAAV8, are capable of transducing the liver when delivered systemically in mice, NHPs and humans. See, e.g., Li et al. (2020) Nat. Rev. Genet. 21:255-272, herein incorporated by reference in its entirety for all purposes.

Once in the nucleus, the ssDNA genome is released from the virion and a complementary DNA strand is synthesized to generate a double-stranded DNA (dsDNA) molecule. Double-stranded AAV genomes naturally circularize via their ITRs and become episomes which will persist extrachromosomally in the nucleus. Therefore, for episomal gene therapy programs, rAAV-delivered rAAV episomes provide long-term, promoter-driven gene expression in non-dividing cells. However, this rAAV-delivered episomal DNA is diluted out as cells divide. In contrast, the gene therapy described herein is based on gene insertion to allow long-term gene expression.

When specific rAAVs comprising specific sequences (e.g., specific bidirectional construct sequences or specific unidirectional construct sequences) are disclosed herein, they are meant to encompass the sequence disclosed or the reverse complement of the sequence. For example, if a bidirectional or unidirectional construct disclosed herein consists of the hypothetical sequence 5′-CTGGACCGA-3′, it is also meant to encompass the reverse complement of that sequence (5′-TCGGTCCAG-3′). Likewise, when rAAVs comprising bidirectional or unidirectional construct elements in a specific 5′ to 3′ order are disclosed herein, they are also meant to encompass the reverse complement of the order of those elements. For example, if an rAAV is disclosed herein that comprises a bidirectional construct that comprises from 5′ to 3′ a first splice acceptor, a first coding sequence, a first terminator, a reverse complement of a second terminator, a reverse complement of a second coding sequence, and a reverse complement of a second splice acceptor, it is also meant to encompass a construct comprising from 5′ to 3′ the second splice acceptor, the second coding sequence, the second terminator, a reverse complement of the first terminator, a reverse complement of the first coding sequence, and a reverse complement of the first splice acceptor. Single-stranded AAV genomes are packaged as either sense (plus-stranded) or anti-sense (minus-stranded genomes), and single-stranded AAV genomes of + and − polarity are packaged with equal frequency into mature rAAV virions. See, e.g., LING et al. (2015) J. Mol. Genet. Med. 9(3):175, Zhou et al. (2008) Mol. Ther. 16(3):494-499, and Samulski et al. (1987) J. Virol. 61:3096-3101, each of which is herein incorporated by reference in its entirety for all purposes.

The ssDNA AAV genome consists of two open reading frames, Rep and Cap, flanked by two inverted terminal repeats that allow for synthesis of the complementary DNA strand. When constructing an AAV transfer plasmid, the transgene is placed between the two ITRs, and Rep and Cap can be supplied in trans. In addition to Rep and Cap, AAV can require a helper plasmid containing genes from adenovirus. These genes (E4, E2a, and VA) mediate AAV replication. For example, the transfer plasmid, Rep/Cap, and the helper plasmid can be transfected into HEK293 cells containing the adenovirus gene E1+ to produce infectious AAV particles. Alternatively, the Rep, Cap, and adenovirus helper genes may be combined into a single plasmid. Similar packaging cells and methods can be used for other viruses, such as retroviruses.

Multiple serotypes of AAV have been identified. These serotypes differ in the types of cells they infect (i.e., their tropism), allowing preferential transduction of specific cell types. The term AAV includes, for example, AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “AAV vector” as used herein refers to an AAV vector comprising a heterologous sequence not of AAV origin (i.e., a nucleic acid sequence heterologous to AAV), typically comprising a sequence encoding an exogenous polypeptide of interest. The construct may comprise an AAV1, AAV2, AAV3, AAV3B, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK03, AV10, AAV11, AAV12, rh10, and hybrids thereof, avian AAV, bovine AAV, canine AAV, equine AAV, primate AAV, non-primate AAV, and ovine AAV capsid sequence. In general, the heterologous nucleic acid sequence (the transgene) is flanked by at least one, and generally by two, AAV inverted terminal repeat sequences (ITRs). An AAV vector may either be single-stranded (ssAAV) or self-complementary (scAAV). Examples of serotypes for liver tissue include AAV3B, AAV5, AAV6, AAV7, AAV8, AAV9, AAVrh.74, and AAVhu.37, and particularly AAV8. In a specific example, the AAV vector comprising the nucleic acid construct can be recombinant AAV8 (rAAV8). A rAAV8 vector as described herein is one in which the capsid is from AAV8. For example, an AAV vector using ITRs from AAV2 and a capsid of AAV8 is considered herein to be a rAAV8 vector.

Tropism can be further refined through pseudotyping, which is the mixing of a capsid and a genome from different viral serotypes. For example, AAV2/5 indicates a virus containing the genome of serotype 2 packaged in the capsid from serotype 5. Use of pseudotyped viruses can improve transduction efficiency, as well as alter tropism. Hybrid capsids derived from different serotypes can also be used to alter viral tropism. For example, AAV-DJ contains a hybrid capsid from eight serotypes and displays high infectivity across a broad range of cell types in vivo. AAV-DJ8 is another example that displays the properties of AAV-DJ but with enhanced brain uptake. AAV serotypes can also be modified through mutations. Examples of mutational modifications of AAV2 include Y444F, Y500F, Y730F, and S662V. Examples of mutational modifications of AAV3 include Y705F, Y731F, and T492V. Examples of mutational modifications of AAV6 include S663V and T492V. Other pseudotyped/modified AAV variants include AAV2/1, AAV2/6, AAV2/7, AAV2/8, AAV2/9, AAV2.5, AAV8.2, and AAV/SASTG.

To accelerate transgene expression, self-complementary AAV (scAAV) variants can be used. Because AAV depends on the cell's DNA replication machinery to synthesize the complementary strand of the AAV's single-stranded DNA genome, transgene expression may be delayed. To address this delay, scAAV containing complementary sequences that are capable of spontaneously annealing upon infection can be used, eliminating the requirement for host cell DNA synthesis. However, single-stranded AAV (ssAAV) vectors can also be used.

To increase packaging capacity, longer transgenes may be split between two AAV transfer plasmids, the first with a 3′ splice donor and the second with a 5′ splice acceptor. Upon co-infection of a cell, these viruses form concatemers, are spliced together, and the full-length transgene can be expressed. Although this allows for longer transgene expression, expression is less efficient. Similar methods for increasing capacity utilize homologous recombination. For example, a transgene can be divided between two transfer plasmids but with substantial sequence overlap such that co-expression induces homologous recombination and expression of the full-length transgene.

The vector (e.g., AAV such as recombinant AAV8) can be formulated, for example, in 10 mM sodium phosphate, 180 mM sodium chloride, and 0.005% poloxamer 188, at pH 7.3.

In certain AAVs, the cargo can include nucleic acids encoding one or more guide RNAs (e.g., DNA encoding a guide RNA, or DNA encoding two or more guide RNAs). In certain AAVs, the cargo can include a nucleic acid (e.g., DNA) encoding a Cas nuclease, such as Cas9, and DNA encoding one or more guide RNAs (e.g., DNA encoding a guide RNA, or DNA encoding two or more guide RNAs). In certain AAVs, the cargo can include a nucleic acid construct encoding a polypeptide of interest. In certain AAVs, the cargo can include a nucleic acid (e.g., DNA) encoding a Cas nuclease, such as Cas9, a DNA encoding a guide RNA (or multiple guide RNAs), and a nucleic acid construct encoding a polypeptide of interest.

For example, Cas or Cas9 and one or more gRNAs (e.g., 1 gRNA or 2 gRNAs or 3 gRNAs or 4 gRNAs) can be delivered via LNP-mediated delivery (e.g., in the form of RNA) or adeno-associated virus (AAV)-mediated delivery (e.g., rAAV8-mediated delivery). For example, a Cas9 mRNA and a gRNA can be delivered via LNP-mediated delivery, or DNA encoding Cas9 and DNA encoding a gRNA can be delivered via AAV-mediated delivery. The Cas or Cas9 and the gRNA(s) can be delivered in a single AAV or via two separate AAVs. For example, a first AAV can carry a Cas or Cas9 expression cassette, and a second AAV can carry a gRNA expression cassette. Similarly, a first AAV can carry a Cas or Cas9 expression cassette, and a second AAV can carry two or more gRNA expression cassettes. Alternatively, a single AAV can carry a Cas or Cas9 expression cassette (e.g., Cas or Cas9 coding sequence operably linked to a promoter) and a gRNA expression cassette (e.g., gRNA coding sequence operably linked to a promoter). Similarly, a single AAV can carry a Cas or Cas9 expression cassette (e.g., Cas or Cas9 coding sequence operably linked to a promoter) and two or more gRNA expression cassettes (e.g., gRNA coding sequences operably linked to promoters). Different promoters can be used to drive expression of the gRNA, such as a U6 promoter or the small tRNA Gln. Likewise, different promoters can be used to drive Cas9 expression. For example, small promoters are used so that the Cas9 coding sequence can fit into an AAV construct. Similarly, small Cas9 proteins (e.g., SaCas9 or CjCas9 are used to maximize the AAV packaging capacity).

### XVII. Methods of Introducing, Integrating, or Expressing a Nucleic Acid Encoding a Polypeptide of Interest in Cells or Subjects and Therapeutic Methods

The plasma cell depleting agents or combinations comprising plasma cell depleting agents, nucleic acid constructs, nuclease agents and CRISPR/Cas systems, and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of treating an enzyme deficiency in a subject, and methods or preventing or reducing the onset of a sign or symptom an enzyme deficiency in a subject. In some embodiments, the subject has preexisting immunity against the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent. For example, the delivery vehicle can be a recombinant AAV (e.g., AAV comprising a nucleic acid construct described herein). Suitable combinations comprising a plasma cell depleting agent are described in more detail elsewhere herein. In one example, the enzyme deficiency is FIX deficiency or the disease is hemophilia B. In another example, the enzyme deficiency is GAA deficiency or the disease is Pompe disease. In another example, the enzyme deficiency is FVIII deficiency or the disease is hemophilia A. In other methods (e.g., where a nucleic acid construct encodes a neutralizing antigen-binding protein as disclosed herein), the methods can be for treating an infectious disease (e.g., bacterial or viral) or treating a cancer. The plasma cell depleting agents or combinations comprising plasma cell depleting agents can inhibit or prevent an immune response to an immunogen (e.g., nucleic acid construct, nuclease agent or CRISPR/Cas system, e.g., in an immunogenic delivery vehicle) (e.g., an immunogenic delivery vehicle) in a subject in need thereof or can inhibit or prevent generation of antibodies (e.g., neutralizing antibodies) to an immunogen (e.g., nucleic acid construct, nuclease agent or CRISPR/Cas system, e.g., in an immunogenic delivery vehicle) (e.g., an immunogenic delivery vehicle) in a subject in need thereof. The term “immune response” refers to a response of a cell of the immune system (e.g., a B-cell, T-cell, macrophage or polymorphonucleocyte) to a stimulus such as an immunogen, e.g., an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host. In some embodiments, the immune response is a humoral (antibody producing) immune response and/or a cell-mediated immune response in a subject (e.g., a human).

An antibody may be capable of both binding and neutralizing a viral particle or a portion thereof (e.g., neutralizing antibody (nAb)). In some embodiments, the antibody may affect pharmacokinetic properties or alter uptake of AAV into different cell types. In some embodiments, neutralizing (or neutralize or neutralization and the like) in the context of the present disclosure may comprise an effect of immunoglobulins, such as antibodies generated in a host immune response, in reducing the efficacy and/or delivery of a viral particle. As an example, neutralization by an at least one nAb described herein may be realized such that the nAb is directed to the viral particle surface (e.g., capsid protein) which may result in aggregation of viral particles and/or may be realized by inhibition of the fusion of viral and a cellular membrane(s) after attachment of the viral particle to a target cell, by inhibition of endocytosis, and/or by inhibition of production of viral progeny. In various embodiments, an antibody generated in a subject's host immune response can play a neutralizing role thereby causing the delivery effectiveness of the viral particle to be reduced or eliminated. In some embodiments, the induced and/or preexisting host immunity may comprise B and/or T cell immune responses described herein. The blockade and/or suppression of induced and/or preexisting host immunity against viral particles or portions thereof, can improve viral transduction and allow for effective re-administration (i.e., re-dosing) of the viral particles during gene therapy.

In any of the methods, the subject can be from any suitable species, such as eukaryotic or mammalian subjects (e.g., non-human mammalian subject or human subject). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples include, but are not limited to, humans, rodents, mice, rats, and non-human primates. In a specific example, the subject is a human. Likewise, cells can be any suitable type of cell. In a specific example, the cell or cells are a liver cell or liver cells such as a hepatocyte or hepatocytes (e.g., human liver cell(s) or human hepatocyte(s)).

The subjects can be neonatal subjects in some methods. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject. In other methods, the subjects are not neonatal subjects.

In one example, provided herein are methods of introducing a nucleic acid encoding a polypeptide of interest into a cell or a population of cells in a subject. Such methods can comprise administering any of the nucleic acid constructs described herein (or any of the compositions comprising a nucleic acid construct described herein, including, for example, vectors or lipid nanoparticles) to the subject in combination with a plasma cell depleting agents or combination comprising a plasma cell depleting agent. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. The nucleic acid construct can be administered together with a nuclease agent described herein, or can be administered alone. For example, the nucleic acid construct can be one that expresses the polypeptide of interest without being integrated into target genomic locus (e.g., an episomal vector or an expression vector in which the coding sequence for the polypeptide of interest is operably linked to a promoter). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

In another example, provided herein are methods of expressing a polypeptide of interest (e.g., from a target genomic locus) in a cell or a population of cells in a subject. Such methods can comprise administering any of the nucleic acid constructs described herein (or any of the compositions comprising a nucleic acid construct described herein, including, for example, vectors or lipid nanoparticles) to the subject in combination with a plasma cell depleting agent or combination comprising a plasma cell depleting agent. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

In another example, provided herein are methods of inserting or integrating a nucleic acid construct into a target genomic locus in a cell or a population of cells in a subject. Such methods can comprise administering any of the nucleic acid constructs described herein (or any of the compositions comprising a nucleic acid construct described herein, including, for example, vectors or lipid nanoparticles) to the subject in combination with a plasma cell depleting agent or combination comprising the plasma cell depleting agent. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

Also provided are methods of treating an enzyme deficiency in a subject in need thereof. Such methods can comprise administering any of the nucleic acid constructs described herein (or any of the compositions comprising a nucleic acid construct described herein, including, for example, vectors or lipid nanoparticles) to the subject in combination with a plasma cell depleting agent or combination comprising the plasma cell depleting agent such that a therapeutically effective level of polypeptide of interest expression or a therapeutically effective level of circulating polypeptide of interest is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and the polypeptide of interest can be expressed from the modified ALB gene (e.g., such that a therapeutically effective level of polypeptide of interest expression or a therapeutically effective level of circulating polypeptide of interest is achieved in the subject). In one example, the subject has a disease of a bleeding disorder characterized by the enzyme deficiency, a disease of an inborn error of metabolism characterized by the enzyme deficiency, or a lysosomal storage disease characterized by the enzyme deficiency. In one example, the disease is hemophilia B and the polypeptide of interest is a factor IX protein. In another example, the disease is hemophilia A and the polypeptide of interest is a factor VIII protein. In another example, the disease is Pompe disease and the polypeptide of interest is a multidomain therapeutic protein comprising a delivery domain fused to a lysosomal alpha-glucosidase.

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease.

Also provided are methods of preventing or reducing the onset of a sign or symptom of an enzyme deficiency in a subject (e.g., as compared to an untreated, control subject). By preventing is meant the sign or symptom of the enzyme deficiency never becomes present. Such methods can comprise administering any of the nucleic acid constructs described herein (or any of the compositions comprising a nucleic acid construct described herein, including, for example, vectors or lipid nanoparticles) to the subject in combination with a plasma cell depleting agent or combination comprising the plasma cell depleting agent such that a therapeutically effective level of polypeptide of interest expression or a therapeutically effective level of circulating polypeptide of interest is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and the polypeptide of interest can be expressed from the modified ALB gene (e.g., such that a therapeutically effective level of polypeptide of interest expression or a therapeutically effective level of circulating polypeptide of interest is achieved in the subject). In one example, the subject has a disease of a bleeding disorder characterized by the enzyme deficiency, a disease of an inborn error of metabolism characterized by the enzyme deficiency, or a lysosomal storage disease characterized by the enzyme deficiency. In one example, the disease is hemophilia B and the polypeptide of interest is a factor IX protein. In another example, the disease is hemophilia A and the polypeptide of interest is a factor VIII protein. In another example, the disease is Pompe disease and the polypeptide of interest is a multidomain therapeutic protein comprising a delivery domain fused to a lysosomal alpha-glucosidase.

Any of the above methods can further comprise one or more subsequent administration steps. The subsequent administration step can comprise, for example, administering the nucleic acid construct to the subject one or more subsequent times until a desired level of expression and/or activity of the polypeptide of interest is achieved in the subject. In one example, the subsequent administration step can comprise administering to the subject one or more subsequent times: (a) the nucleic acid construct; (b) the nuclease agent or the one or more nucleic acids encoding the nuclease agent; and optionally (c) the plasma cell depleting agent or combination comprising the plasma cell depleting agent. In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the plasma cell depleting agent or combination comprising the plasma cell depleting agent. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 255 (e.g., G009860). For example, the first nuclease agent can target SEQ ID NO: 255 (e.g., G009860), and the second nuclease agent can target SEQ ID NO: 249 (e.g., G009844) (or vice versa). For example, the first nuclease agent can target SEQ ID NO: 255 (e.g., G009860), and the second nuclease agent can target SEQ ID NO: 252 (e.g., G009857) (or vice versa). For example, the first nuclease agent can target SEQ ID NO: 255 (e.g., G009860), and the second nuclease agent can target SEQ ID NO: 260 (e.g., G009874) (or vice versa). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the plasma cell depleting agent or combination comprising the plasma cell depleting agent. In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing). Such methods can further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest in the subject. The measuring can be, for example, at least 1 week, at least 2 weeks, at least 3 weeks, or at least 4 weeks after dosing (e.g., at least 4 weeks after dosing) or can be from about 1 week to about 7 weeks, about 2 weeks to about 6 weeks, about 3 weeks to about 5 weeks, about 4 weeks, about 1 week to about 4 weeks, about 2 weeks to about 4 weeks, about 3 weeks to about 4 weeks, about 4 weeks to about 5 weeks, about 4 weeks to about 6 weeks, or about 4 weeks to about 7 weeks after dosing. In one specific example, the polypeptide of interest is a factor IX protein, and the desired expression level of the factor IX protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL or about 1 to about 5, about 2 to about 5, or about 3 to about 5 μg/mL (e.g., a serum level of at least about 3 μg/mL or about 3 to about 5 μg/mL). In another specific example, the polypeptide of interest is a multidomain therapeutic protein comprising a delivery domain fused to a lysosomal alpha-glucosidase, and the desired expression level of the multidomain therapeutic protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL (e.g., a serum level of at least about 2 μg/mL or at least about 5 μg/mL or about 2 to about 50 μg/mL).

Alternatively, the subsequent administration step can comprise, for example, administering a second nucleic acid construct comprising a second coding sequence for the polypeptide of interest (e.g., wherein the second coding sequence is different from the first coding sequence) to the subject one or more subsequent times until a desired level of expression and/or activity of the polypeptide of interest is achieved in the subject. In one example, the subsequent administration step can comprise administering to the subject one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the plasma cell depleting agent or combination comprising the plasma cell depleting agent. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 255 (e.g., G009860). For example, the first nuclease agent can target SEQ ID NO: 255 (e.g., G009860), and the second nuclease agent can target SEQ ID NO: 249 (e.g., G009844) (or vice versa). For example, the first nuclease agent can target SEQ ID NO: 255 (e.g., G009860), and the second nuclease agent can target SEQ ID NO: 252 (e.g., G009857) (or vice versa). For example, the first nuclease agent can target SEQ ID NO: 255 (e.g., G009860), and the second nuclease agent can target SEQ ID NO: 260 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing). Such methods can further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest in the subject. The measuring can be, for example, at least 1 week, at least 2 weeks, at least 3 weeks, or at least 4 weeks after dosing (e.g., at least 4 weeks after dosing) or can be from about 1 week to about 7 weeks, about 2 weeks to about 6 weeks, about 3 weeks to about 5 weeks, about 4 weeks, about 1 week to about 4 weeks, about 2 weeks to about 4 weeks, about 3 weeks to about 4 weeks, about 4 weeks to about 5 weeks, about 4 weeks to about 6 weeks, or about 4 weeks to about 7 weeks after dosing. In one specific example, the polypeptide of interest is a factor IX protein, and the desired expression level of the factor IX protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL or about 1 to about 5, about 2 to about 5, or about 3 to about 5 μg/mL (e.g., a serum level of at least about 3 μg/mL or at least about 5 μg/mL or about 3-5 μg/mL). In another specific example, the polypeptide of interest is a multidomain therapeutic protein comprising a delivery domain fused to a lysosomal alpha-glucosidase, and the desired expression level of the multidomain therapeutic protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL (e.g., a serum level of at least about 2 μg/mL or at least about 5 μg/mL).

Alternatively, the subsequent administration step can comprise, for example, administering a second nucleic acid construct comprising a coding sequence for a second polypeptide of interest (e.g., that is different from the first polypeptide of interest) to the subject one or more subsequent times until a desired level of expression and/or activity of the polypeptide of interest is achieved in the subject. In one example, the subsequent administration step can comprise administering to the subject one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the plasma cell depleting agent or combination comprising the plasma cell depleting agent. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 255 (e.g., G009860). For example, the first nuclease agent can target SEQ ID NO: 255 (e.g., G009860), and the second nuclease agent can target SEQ ID NO: 249 (e.g., G009844) (or vice versa). For example, the first nuclease agent can target SEQ ID NO: 255 (e.g., G009860), and the second nuclease agent can target SEQ ID NO: 252 (e.g., G009857) (or vice versa). For example, the first nuclease agent can target SEQ ID NO: 255 (e.g., G009860), and the second nuclease agent can target SEQ ID NO: 260 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

In some methods, the one or more subsequent administration steps is one subsequent administration step. In some methods, the one or more subsequent administration steps is two subsequent administration steps or comprises at least two subsequent administration steps. In some methods, the one or more subsequent administration steps is three subsequent administration steps or comprises at least three subsequent administration steps. In some methods, the one or more subsequent administration steps is four subsequent administration steps or comprises at least four subsequent administration steps.

In some methods, the plasma cell depleting agent or combination comprising the plasma cell depleting agent is administered in the one or more subsequent administration steps if there is no plasma cell depleting agent or combination comprising the plasma cell depleting agent in the subject. In some methods, the plasma cell depleting agent or combination comprising the plasma cell depleting agent is administered in the one or more subsequent administration steps if preexisting expression and/or activity levels of plasma cell depleting agent or combination comprising the plasma cell depleting agent are below a desired threshold level (i.e., the level necessary to achieve the desired effect). In some methods, the method comprises measuring expression and/or activity levels of the plasma cell depleting agent or combination comprising the plasma cell depleting agent prior to the one or more subsequent administration steps.

In some methods, a therapeutically effective amount of the nucleic acid construct or the composition comprising the nucleic acid construct or the combination of the nucleic acid construct and the plasma cell depleting agent or combination comprising the plasma cell depleting agent and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. Use multiple administration steps can, in some methods, enable use of lower doses of nucleic acid construct and/or nuclease agent for administration to the subject as compared to methods in which only a single administration step is used. For example, if 2-3 administration steps are used, the dose of nucleic acid construct and/or nuclease agent can, in some methods, be 2-3× lower than the dose used in methods in which only a single administration step is used.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

In methods in which a nucleic acid construct is genomically integrated, any target genomic locus capable of expressing a gene can be used, such as a safe harbor locus (safe harbor gene) or an endogenous locus that would normally encode the polypeptide of interest (e.g., a F9 locus for Factor IX). Such loci are described in more detail elsewhere herein. In a specific example, the target genomic locus can be an endogenous ALB locus, such as an endogenous human ALB locus. For example, the nucleic acid construct can be genomically integrated in intron 1 of the endogenous ALB locus. Endogenous ALB exon 1 can then splice into the coding sequence for the multidomain therapeutic protein in the nucleic acid construct.

Targeted insertion of the nucleic acid construct comprising the coding sequence for the polypeptide of interest into a target genomic locus, and particularly an endogenous ALB locus, offers multiple advantages. Such methods result in stable modification to allow for stable, long-term expression of the coding sequence for the polypeptide of interest. With respect to the ALB locus, such methods are able to utilize the endogenous ALB promoter and regulatory regions to achieve therapeutically effective levels of expression. For example, the coding sequence for the polypeptide of interest in the nucleic acid construct can comprise a promoterless gene, and the inserted nucleic acid construct can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the coding sequence for the polypeptide of interest in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the multidomain therapeutic protein coding sequence from the nucleic acid construct. Alternatively, in some methods, none of the endogenous gene at the target genomic locus is expressed. As one example, the modified target genomic locus (e.g., modified ALB locus) after integration of the nucleic acid construct can encode a chimeric protein comprising an endogenous secretion signal (e.g., albumin secretion signal) and the polypeptide of interest encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the coding sequence for the polypeptide of interest will support expression and secretion of the polypeptide of interest. Splicing between endogenous ALB exon 1 and the integrated coding sequence for the polypeptide of interest creates a chimeric mRNA and protein including the endogenous ALB sequence encoded by exon 1 operably linked to the coding sequence for the polypeptide of interest encoded by the integrated nucleic acid construct.

The nucleic acid construct can be inserted into the target genomic locus by any means, including homologous recombination (HR) and non-homologous end joining (NHEJ) as described elsewhere herein. In a specific example, the nucleic acid construct is inserted by NHEJ (e.g., does not comprise a homology arm and is inserted by NHEJ).

In another specific example, the nucleic acid construct can be inserted via homology-independent targeted integration (e.g., directional homology-independent targeted integration). For example, the coding sequence for the polypeptide of interest in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the coding sequence for the polypeptide of interest. In a specific example, the nucleic acid construct is delivered AAV-mediated delivery, and cleavage of the target sites flanking the coding sequence for the polypeptide of interest can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the coding sequence for the polypeptide of interest is inserted into the target genomic locus in the correct orientation but it is reformed if the coding sequence for the polypeptide of interest is inserted into the target genomic locus in the opposite orientation. This can help ensure that the coding sequence for the is inserted in the correct orientation for expression.

In any of the above methods, the plasma cell depleting agent or combination comprising the plasma cell depleting agent can be administered simultaneously with the nucleic acid construct and/or nuclease agent (e.g., CRISPR/Cas system) or not simultaneously (e.g., sequentially in any combination). For example, in a method comprising administering a composition or combination comprising a plasma cell depleting agent or combination comprising the plasma cell depleting agent, a nucleic acid construct, and a nuclease agent, they can be administered separately (e.g., the plasma cell depleting agent or combination comprising the plasma cell depleting agent separately from the nucleic acid construct and/or nuclease agent). For example, the plasma cell depleting agent or combination comprising the plasma cell depleting agent can be administered prior to the nucleic acid construct and/or nuclease agent, subsequent to the nucleic acid construct and/or nuclease agent, prior to and subsequent to the nucleic acid construct and/or nuclease agent, or at the same time as the nucleic acid construct and/or nuclease agent. Any suitable methods of administering plasma cell depleting agents or combinations comprising a plasma cell depleting agent, nucleic acid constructs, and nuclease agents to cells can be used (particularly methods of administering to the liver for the nucleic acid constructs and nuclease agents), and examples of such methods are described in more detail elsewhere herein.

In methods in which a composition or combination comprising a plasma cell depleting agent or combination comprising the plasma cell depleting agent, a nucleic acid construct (or vector or LNP), and a nuclease agent is administered (i.e., in methods in which a plasma cell depleting agent or combination comprising the plasma cell depleting agent, a nucleic acid construct (or vector or LNP), and a nuclease agent are both administered), the plasma cell depleting agent or combination comprising the plasma cell depleting agent, the nucleic acid construct, and the nuclease agent can be administered simultaneously. Alternatively, the plasma cell depleting agent or combination comprising the plasma cell depleting agent and the nucleic acid construct and the nuclease agent can be administered sequentially in any order. For example, the plasma cell depleting agent or combination comprising the plasma cell depleting agent can be administered prior to and/or after the nucleic acid construct and/or nuclease agent. In one example, the plasma cell depleting agent or combination comprising the plasma cell depleting agent can be administered prior to and after the nucleic acid construct and/or nuclease agent. In another example, the plasma cell depleting agent or combination comprising the plasma cell depleting agent can be administered simultaneously with and after the nucleic acid construct and/or nuclease agent.

In one example, the plasma cell depleting agent or combination comprising the plasma cell depleting agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the nucleic acid construct and/or nuclease agent.

In one example, the plasma cell depleting agent or combination comprising the plasma cell depleting agent is administered about 4 hours, about 8 hours, about 12 hours, about 18 hours, about 1 day, about 2 days, about 3 days, about 4 days, about 5 days, about 6 days, or about 1 week prior to administering the nucleic acid construct and/or nuclease agent. In another example, the plasma cell depleting agent or combination comprising the plasma cell depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nucleic acid construct and/or nuclease agent. In another example, the plasma cell depleting agent or combination comprising the plasma cell depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nucleic acid construct and/or nuclease agent.

In one example, the plasma cell depleting agent or combination comprising the plasma cell depleting agent is administered about 1 week prior to administering the nucleic acid construct and/or nuclease agent. In another example, the plasma cell depleting agent or combination comprising the plasma cell depleting agent is administered within about 1 week prior to administering the nucleic acid construct and/or nuclease agent. In another example, the plasma cell depleting agent or combination comprising the plasma cell depleting agent is administered at least about 1 day, at least about 2 days, at least about 3 days, at least about 4 days, at least about 5 days, at least about 6 days, or at least about 1 week prior to administering the nucleic acid construct and/or nuclease agent. In another example, the plasma cell depleting agent or combination comprising the plasma cell depleting agent is administered about 1 day, about 2 days, about 3 days, about 4 days, about 5 days, about 6 days, or about 1 week prior to administering the nucleic acid construct and/or nuclease agent. In another example, the plasma cell depleting agent or combination comprising the plasma cell depleting agent is administered about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, or about 6 days to about 7 days prior to administering the nucleic acid construct and/or nuclease agent.

In one example, the plasma cell depleting agent or combination comprising the plasma cell depleting agent is administered about 4 hours, about 8 hours, about 12 hours, about 18 hours, about 1 day, about 2 days, about 3 days, about 4 days, about 5 days, about 6 days, or about 1 week prior to and subsequent to administering the nucleic acid construct and/or nuclease agent. In another example, the plasma cell depleting agent or combination comprising the plasma cell depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering the nucleic acid construct and/or nuclease agent. In another example, the plasma cell depleting agent or combination comprising the plasma cell depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the nucleic acid construct and/or nuclease agent.

In one example, the plasma cell depleting agent or combination comprising the plasma cell depleting agent is administered about 4 hours, about 8 hours, about 12 hours, about 18 hours, about 1 day, about 2 days, about 3 days, about 4 days, about 5 days, about 6 days, or about 1 week after administering the nucleic acid construct and/or nuclease agent. In another example, the plasma cell depleting agent or combination comprising the plasma cell depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nucleic acid construct and/or nuclease agent. In another example, the plasma cell depleting agent or combination comprising the plasma cell depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nucleic acid construct and/or nuclease agent.

In one example, the plasma cell depleting agent or combination comprising the plasma cell depleting agent is administered within about 1 month, within about 2 months, within about 3 months, within about 4 months, within about 5 months, within about 6 months, or within about 12 months after administering the nucleic acid construct and/or nuclease agent. In another example, the plasma cell depleting agent or combination comprising the plasma cell depleting agent is administered within about 6 months after administering the nucleic acid construct and/or nuclease agent. In another example, the plasma cell depleting agent or combination comprising the plasma cell depleting agent is administered about 1 month, about 2 months, about 3 months, about 4 months, about 5 months, or about 6 months after administering the nucleic acid construct and/or nuclease agent. In another example, the plasma cell depleting agent or combination comprising the plasma cell depleting agent is administered about 1 to about 6 months, about 2 to about 6 months, about 3 to about 6 months, about 4 to about 6 months, about 5 to about 6 months, about 1 to about 5 months, about 1 to about 4 months, about 1 to about 3 months, or about 1 to about 2 months after administering the nucleic acid construct and/or nuclease agent. In another example, the plasma cell depleting agent or combination comprising the plasma cell depleting agent is administered more than 6 months after administering the nucleic acid construct and/or nuclease agent. In another example, the plasma cell depleting agent or combination comprising the plasma cell depleting agent is administered about 1 to about 12 months or about 6 months to about 12 months after administering the nucleic acid construct and/or nuclease agent. In some methods, the method can comprise determining whether the nucleic acid construct and/or the nuclease agent are present in the subject (e.g., from a previous administration). The method can then comprise administering the plasma cell depleting agent or combination comprising the plasma cell depleting agent if the nucleic acid construct and/or the nuclease agent are still present in the subject. For example, if the nucleic acid construct is delivered in a viral vector (e.g., a recombinant AAV vector), the method can comprise determining whether the viral vector is present in the subject. The method can then comprise administering the plasma cell depleting agent or combination comprising the plasma cell depleting agent if the viral vector is still present (i.e., detectable) in the subject. For example, if the nucleic acid construct is delivered in a viral vector (e.g., a recombinant AAV vector), the method can comprise determining whether viral capsid protein (e.g., AAV capsid protein) is present in the subject. The method can then comprise administering the plasma cell depleting agent or combination comprising the plasma cell depleting agent if the capsid protein is still present (i.e., detectable) in the subject. For example, if the nuclease agent is delivered in a lipid nanoparticle, the method can comprise determining whether the lipid nanoparticle components (e.g., PEG) are present in the subject. The method can then comprise administering the plasma cell depleting agent or combination comprising the plasma cell depleting agent if the lipid nanoparticle components are still present (i.e., detectable) in the subject. For example, if the nuclease agent is delivered in a lipid nanoparticle, the method can comprise determining whether certain lipid nanoparticle components are present in the subject. The method can then comprise administering the plasma cell depleting agent or combination comprising the plasma cell depleting agent if the components still present (i.e., detectable) in the subject.

The B cell depleting agents (e.g., anti-CD20×CD3 antigen binding molecules), nucleic acid constructs, nuclease agents and CRISPR/Cas systems, and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of treating an enzyme deficiency in a subject, and methods or preventing or reducing the onset of a sign or symptom an enzyme deficiency in a subject. In some embodiments, the subject does not have preexisting immunity against the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent. In one example, the enzyme deficiency is FIX deficiency or the disease is hemophilia B. In another example, the enzyme deficiency is GAA deficiency or the disease is Pompe disease. In another example, the enzyme deficiency is FVIII deficiency or the disease is hemophilia A. In other methods (e.g., where a nucleic acid construct encodes a neutralizing antigen-binding protein as disclosed herein), the methods can be for treating an infectious disease (e.g., bacterial or viral) or treating a cancer. The B cell depleting agents can inhibit or prevent an immune response to an immunogen (e.g., nucleic acid construct, nuclease agent or CRISPR/Cas system, e.g., in an immunogenic delivery vehicle) (e.g., an immunogenic delivery vehicle) in a subject in need thereof or can inhibit or prevent generation of antibodies (e.g., neutralizing antibodies) to an immunogen (e.g., nucleic acid construct, nuclease agent or CRISPR/Cas system, e.g., in an immunogenic delivery vehicle) (e.g., an immunogenic delivery vehicle) in a subject in need thereof. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, an immunoglobulin depleting agent is not administered to the subject (e.g., a subject without preexisting immunity against the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. The term “immune response” refers to a response of a cell of the immune system (e.g., a B-cell, T-cell, macrophage or polymorphonucleocyte) to a stimulus such as an immunogen, e.g., an antigen (e.g., a viral antigen). Active immune responses can involve differentiation and proliferation of immunocompetent cells, which leads to synthesis of antibodies or the development of cell-mediated reactivity, or both. An active immune response can be mounted by the host after exposure to an antigen (e.g., by infection or by vaccination). Active immune response can be contrasted with passive immunity, which can be acquired through the transfer of substances such as, e.g., an antibody, transfer factor, thymic graft, and/or cytokines from an actively immunized host to a non-immune host. In some embodiments, the immune response is a humoral (antibody producing) immune response and/or a cell-mediated immune response in a subject (e.g., a human).

An antibody may be capable of both binding and neutralizing a viral particle or a portion thereof (e.g., neutralizing antibody (nAb)). In some embodiments, the antibody may affect pharmacokinetic properties or alter uptake of AAV into different cell types. In some embodiments, neutralizing (or neutralize or neutralization and the like) in the context of the present disclosure may comprise an effect of immunoglobulins, such as antibodies generated in a host immune response, in reducing the efficacy and/or delivery of a viral particle. As an example, neutralization by an at least one nAb described herein may be realized such that the nAb is directed to the viral particle surface (e.g., capsid protein) which may result in aggregation of viral particles and/or may be realized by inhibition of the fusion of viral and a cellular membrane(s) after attachment of the viral particle to a target cell, by inhibition of endocytosis, and/or by inhibition of production of viral progeny. In various embodiments, an antibody generated in a subject's host immune response can play a neutralizing role thereby causing the delivery effectiveness of the viral particle to be reduced or eliminated. In some embodiments, the induced and/or preexisting host immunity may comprise B and/or T cell immune responses described herein. The blockade and/or suppression of induced and/or preexisting host immunity against viral particles or portions thereof, can improve viral transduction and allow for effective re-administration (i.e., re-dosing) of the viral particles during gene therapy.

In any of the methods, the subject can be from any suitable species, such as eukaryotic or mammalian subjects (e.g., non-human mammalian subject or human subject). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples include, but are not limited to, humans, rodents, mice, rats, and non-human primates. In a specific example, the subject is a human. Likewise, cells can be any suitable type of cell. In a specific example, the cell or cells are a liver cell or liver cells such as a hepatocyte or hepatocytes (e.g., human liver cell(s) or human hepatocyte(s)).

The subjects can be neonatal subjects in some methods. A neonatal subject can be a human subject up to or under the age of 1 year (52 weeks), preferably up to or under the age of 24 weeks, more preferably up to or under the age of 12 weeks, more preferably up to or under the age of 8 weeks, and even more preferably up to or under the age of 4 weeks. In certain embodiments, a neonatal human subject is up to 4 weeks of age. In certain embodiments, a neonatal human subject is up to 8 weeks of age. In another embodiment, a neonatal human subject is within 3 weeks after birth. In another embodiment, a neonatal human subject is within 2 weeks after birth. In another embodiment, a neonatal human subject is within 1 week after birth. In another embodiment, a neonatal human subject is within 7 days after birth. In another embodiment, a neonatal human subject is within 6 days after birth. In another embodiment, a neonatal human subject is within 5 days after birth. In another embodiment, a neonatal human subject is within 4 days after birth. In another embodiment, a neonatal human subject is within 3 days after birth. In another embodiment, a neonatal human subject is within 2 days after birth. In another embodiment, a neonatal human subject is within 1 day after birth. The time windows disclosed above are for human subjects and are also meant to cover the corresponding developmental time windows for other animals. As used herein, a “neonatal cell” is a cell of a neonatal subject, and a population of neonatal cells is a population of cells of a neonatal subject. In other methods, the subjects are not neonatal subjects.

In one example, provided herein are methods of introducing a nucleic acid encoding a polypeptide of interest into a cell or a population of cells in a subject. Such methods can comprise administering any of the nucleic acid constructs described herein (or any of the compositions comprising a nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with a B cell depleting agent (e.g., anti-CD20×CD3 antigen-binding molecule). In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. The nucleic acid construct can be administered together with a nuclease agent described herein, or can be administered alone. For example, the nucleic acid construct can be one that expresses the polypeptide of interest without being integrated into target genomic locus (e.g., an episomal vector or an expression vector in which the coding sequence for the polypeptide of interest is operably linked to a promoter). In some methods, the nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

In another example, provided herein are methods of expressing a polypeptide of interest (e.g., from a target genomic locus) in a cell or a population of cells in a subject. Such methods can comprise administering any of the nucleic acid constructs described herein (or any of the compositions comprising a nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with a B cell depleting agent (e.g., anti-CD20×CD3 antigen-binding molecule). In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

In another example, provided herein are methods of inserting or integrating a nucleic acid construct into a target genomic locus in a cell or a population of cells in a subject. Such methods can comprise administering any of the nucleic acid constructs described herein (or any of the compositions comprising a nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with a B cell depleting agent (e.g., anti-CD20×CD3 antigen-binding molecule). In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the nucleic acid construct or composition comprising the nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus. The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence, the nucleic acid construct can be inserted into the ALB gene to create a modified ALB gene, and the polypeptide of interest can be expressed from the modified ALB gene.

Also provided are methods of treating an enzyme deficiency in a subject in need thereof. Such methods can comprise administering any of the nucleic acid constructs described herein (or any of the compositions comprising a nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with a B cell depleting agent (e.g., anti-CD20×CD3 antigen-binding molecule) such that a therapeutically effective level of polypeptide of interest expression or a therapeutically effective level of circulating polypeptide of interest is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of polypeptide of interest without integration into a target genomic locus). In some methods, the nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and the polypeptide of interest can be expressed from the modified ALB gene (e.g., such that a therapeutically effective level of polypeptide of interest expression or a therapeutically effective level of circulating polypeptide of interest is achieved in the subject). In one example, the subject has a disease of a bleeding disorder characterized by the enzyme deficiency, a disease of an inborn error of metabolism characterized by the enzyme deficiency, or a lysosomal storage disease characterized by the enzyme deficiency. In one example, the disease is hemophilia B and the polypeptide of interest is a factor IX protein. In another example, the disease is hemophilia A and the polypeptide of interest is a factor VIII protein. In another example, the disease is Pompe disease and the polypeptide of interest is a multidomain therapeutic protein comprising a delivery domain fused to a lysosomal alpha-glucosidase.

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease.

Also provided are methods of preventing or reducing the onset of a sign or symptom of an enzyme deficiency in a subject (e.g., as compared to an untreated, control subject). By preventing is meant the sign or symptom of the enzyme deficiency never becomes present. Such methods can comprise administering any of the nucleic acid constructs described herein (or any of the compositions comprising a nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with a B cell depleting agent (e.g., anti-CD20×CD3 antigen-binding molecule) such that a therapeutically effective level of polypeptide of interest expression or a therapeutically effective level of circulating polypeptide of interest is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the nucleic acid construct or composition comprising the nucleic acid construct can be administered without a nuclease agent (e.g., if the nucleic acid construct comprises elements needed for expression of the polypeptide of interest without integration into a target genomic locus). In some methods, the nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the nucleic acid construct can be inserted into the target genomic locus to create a modified target genomic locus, and the polypeptide of interest can be expressed from the modified target genomic locus (e.g., such that a therapeutically effective level of polypeptide of interest expression or a therapeutically effective level of circulating polypeptide of interest is achieved in the subject). The coding sequence for the polypeptide of interest can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and the polypeptide of interest can be expressed from the modified ALB gene (e.g., such that a therapeutically effective level of polypeptide of interest expression or a therapeutically effective level of circulating polypeptide of interest is achieved in the subject). In one example, the subject has a disease of a bleeding disorder characterized by the enzyme deficiency, a disease of an inborn error of metabolism characterized by the enzyme deficiency, or a lysosomal storage disease characterized by the enzyme deficiency. In one example, the disease is hemophilia B and the polypeptide of interest is a factor IX protein. In another example, the disease is hemophilia A and the polypeptide of interest is a factor VIII protein. In another example, the disease is Pompe disease and the polypeptide of interest is a multidomain therapeutic protein comprising a delivery domain fused to a lysosomal alpha-glucosidase.

Any of the above methods can further comprise one or more subsequent administration steps. The subsequent administration step can comprise, for example, administering the nucleic acid construct to the subject one or more subsequent times until a desired level of expression and/or activity of the polypeptide of interest is achieved in the subject. In one example, the subsequent administration step can comprise administering to the subject one or more subsequent times: (a) the nucleic acid construct; (b) the nuclease agent or the one or more nucleic acids encoding the nuclease agent; and optionally (c) the B cell depleting agent. In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in the target genomic locus, wherein the second nuclease target site is different from the first nuclease target site; and optionally (c) the B cell depleting agent. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 255 (e.g., G009860). For example, the first nuclease agent can target SEQ ID NO: 255 (e.g., G009860), and the second nuclease agent can target SEQ ID NO: 249 (e.g., G009844) (or vice versa). For example, the first nuclease agent can target SEQ ID NO: 255 (e.g., G009860), and the second nuclease agent can target SEQ ID NO: 252 (e.g., G009857) (or vice versa). For example, the first nuclease agent can target SEQ ID NO: 255 (e.g., G009860), and the second nuclease agent can target SEQ ID NO: 260 (e.g., G009874) (or vice versa). In another example, the subsequent administration step can comprise administering to the subject one or more subsequent times: (a) the nucleic acid construct; (b) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the B cell depleting agent. In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing). Such methods can further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest in the subject. The measuring can be, for example, at least 1 week, at least 2 weeks, at least 3 weeks, or at least 4 weeks after dosing (e.g., at least 4 weeks after dosing) or can be from about 1 week to about 7 weeks, about 2 weeks to about 6 weeks, about 3 weeks to about 5 weeks, about 4 weeks, about 1 week to about 4 weeks, about 2 weeks to about 4 weeks, about 3 weeks to about 4 weeks, about 4 weeks to about 5 weeks, about 4 weeks to about 6 weeks, or about 4 weeks to about 7 weeks after dosing. In one specific example, the polypeptide of interest is a factor IX protein, and the desired expression level of the factor IX protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL or about 1 to about 5, about 2 to about 5, or about 3 to about 5 μg/mL (e.g., a serum level of at least about 3 μg/mL or about 3 to about 5 μg/mL). In another specific example, the polypeptide of interest is a multidomain therapeutic protein comprising a delivery domain fused to a lysosomal alpha-glucosidase, and the desired expression level of the multidomain therapeutic protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL (e.g., a serum level of at least about 2 μg/mL or at least about 5 μg/mL or about 2 to about 50 μg/mL).

Alternatively, the subsequent administration step can comprise, for example, administering a second nucleic acid construct comprising a second coding sequence for the polypeptide of interest (e.g., wherein the second coding sequence is different from the first coding sequence) to the subject one or more subsequent times until a desired level of expression and/or activity of the polypeptide of interest is achieved in the subject. In one example, the subsequent administration step can comprise administering to the subject one or more subsequent times: (a) a second nucleic acid construct comprising a second coding sequence for the polypeptide of interest, wherein the second coding sequence is different from the first coding sequence; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the B cell depleting agent. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 255 (e.g., G009860). For example, the first nuclease agent can target SEQ ID NO: 255 (e.g., G009860), and the second nuclease agent can target SEQ ID NO: 249 (e.g., G009844) (or vice versa). For example, the first nuclease agent can target SEQ ID NO: 255 (e.g., G009860), and the second nuclease agent can target SEQ ID NO: 252 (e.g., G009857) (or vice versa). For example, the first nuclease agent can target SEQ ID NO: 255 (e.g., G009860), and the second nuclease agent can target SEQ ID NO: 260 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing). Such methods can further comprise the following steps prior to the subsequent administration step: (i) measuring expression and/or activity of the polypeptide of interest in the subject; and (ii) determining the dose of the nucleic acid construct and the nuclease agent or the one or more nucleic acids encoding the nuclease agent for the subsequent administration step in order to achieve the desired level of expression and/or activity of the polypeptide of interest in the subject. The measuring can be, for example, at least 1 week, at least 2 weeks, at least 3 weeks, or at least 4 weeks after dosing (e.g., at least 4 weeks after dosing) or can be from about 1 week to about 7 weeks, about 2 weeks to about 6 weeks, about 3 weeks to about 5 weeks, about 4 weeks, about 1 week to about 4 weeks, about 2 weeks to about 4 weeks, about 3 weeks to about 4 weeks, about 4 weeks to about 5 weeks, about 4 weeks to about 6 weeks, or about 4 weeks to about 7 weeks after dosing. In one specific example, the polypeptide of interest is a factor IX protein, and the desired expression level of the factor IX protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL or about 1 to about 5, about 2 to about 5, or about 3 to about 5 μg/mL (e.g., a serum level of at least about 3 μg/mL or at least about 5 μg/mL or about 3-5 μg/mL). In another specific example, the polypeptide of interest is a multidomain therapeutic protein comprising a delivery domain fused to a lysosomal alpha-glucosidase, and the desired expression level of the multidomain therapeutic protein in the subject is a serum level of at least about 0.5 μg/mL, at least about 1 μg/mL, at least about 1.5 μg/mL, at least about 2 μg/mL, at least about 2.5 μg/mL, at least about 3 μg/mL, at least about 3.5 μg/mL, at least about 4 μg/mL, at least about 4.5 μg/mL, or at least about 5 μg/mL (e.g., a serum level of at least about 2 μg/mL or at least about 5 μg/mL).

Alternatively, the subsequent administration step can comprise, for example, administering a second nucleic acid construct comprising a coding sequence for a second polypeptide of interest (e.g., that is different from the first polypeptide of interest) to the subject one or more subsequent times until a desired level of expression and/or activity of the polypeptide of interest is achieved in the subject. In one example, the subsequent administration step can comprise administering to the subject one or more subsequent times: (a) a second nucleic acid construct comprising a coding sequence for a second polypeptide of interest that is different from the first polypeptide of interest; (b) (i) the first nuclease agent or the one or more nucleic acids encoding the first nuclease agent; (ii) a second nuclease agent or one or more nucleic acids encoding the second nuclease agent, wherein the second nuclease agent targets a second nuclease target site in the target genomic locus, wherein the second nuclease target site is different from the first nuclease target site; or (iii) a second nuclease agent or one or more nucleic acids encoding the second nuclease agent, wherein the second nuclease agent targets a second nuclease target site in a second target genomic locus that is different from the first target genomic locus (e.g., ALB is the first target genomic locus, and the second target genomic locus is different (e.g., TTR)); and optionally (c) the B cell depleting agent. In one example, the first nuclease target site can be a first location in ALB, and the second nuclease target site can be a second location in ALB. For example, the first nuclease target site can be a first location in intron 1 of ALB, and the second nuclease target site can be a second location in intron 1 of ALB. In a specific example, the first nuclease agent targets SEQ ID NO: 255 (e.g., G009860). For example, the first nuclease agent can target SEQ ID NO: 255 (e.g., G009860), and the second nuclease agent can target SEQ ID NO: 249 (e.g., G009844) (or vice versa). For example, the first nuclease agent can target SEQ ID NO: 255 (e.g., G009860), and the second nuclease agent can target SEQ ID NO: 252 (e.g., G009857) (or vice versa). For example, the first nuclease agent can target SEQ ID NO: 255 (e.g., G009860), and the second nuclease agent can target SEQ ID NO: 260 (e.g., G009874) (or vice versa). In one example, the first target genomic locus can be ALB (e.g., intron 1 of ALB). For example, the first target genomic locus can be ALB (e.g., intron 1 of ALB), and the second target genomic locus can be TTR (e.g., intron 1 of TTR). The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

In some methods, the one or more subsequent administration steps is one subsequent administration step. In some methods, the one or more subsequent administration steps is two subsequent administration steps or comprises at least two subsequent administration steps. In some methods, the one or more subsequent administration steps is three subsequent administration steps or comprises at least three subsequent administration steps. In some methods, the one or more subsequent administration steps is four subsequent administration steps or comprises at least four subsequent administration steps.

In some methods, the B cell depleting agent is administered in the one or more subsequent administration steps if there is no B cell depleting agent in the subject. In some methods, the B cell depleting agent is administered in the one or more subsequent administration steps if preexisting expression and/or activity levels of B cell depleting agent are below a desired threshold level (i.e., the level necessary to achieve the desired effect). In some methods, the method comprises measuring expression and/or activity levels of the B cell depleting agent prior to the one or more subsequent administration steps.

In some methods, a therapeutically effective amount of the nucleic acid construct or the composition comprising the nucleic acid construct or the combination of the nucleic acid construct and the B cell depleting agent and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. Use multiple administration steps can, in some methods, enable use of lower doses of nucleic acid construct and/or nuclease agent for administration to the subject as compared to methods in which only a single administration step is used. For example, if 2-3 administration steps are used, the dose of nucleic acid construct and/or nuclease agent can, in some methods, be 2-3× lower than the dose used in methods in which only a single administration step is used.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311. In certain embodiments, the pharmaceutical compositions are non-pyrogenic.

In methods in which a nucleic acid construct is genomically integrated, any target genomic locus capable of expressing a gene can be used, such as a safe harbor locus (safe harbor gene) or an endogenous locus that would normally encode the polypeptide of interest (e.g., a F9 locus for Factor IX). Such loci are described in more detail elsewhere herein. In a specific example, the target genomic locus can be an endogenous ALB locus, such as an endogenous human ALB locus. For example, the nucleic acid construct can be genomically integrated in intron 1 of the endogenous ALB locus. Endogenous ALB exon 1 can then splice into the coding sequence for the multidomain therapeutic protein in the nucleic acid construct.

Targeted insertion of the nucleic acid construct comprising the coding sequence for the polypeptide of interest into a target genomic locus, and particularly an endogenous ALB locus, offers multiple advantages. Such methods result in stable modification to allow for stable, long-term expression of the coding sequence for the polypeptide of interest. With respect to the ALB locus, such methods are able to utilize the endogenous ALB promoter and regulatory regions to achieve therapeutically effective levels of expression. For example, the coding sequence for the polypeptide of interest in the nucleic acid construct can comprise a promoterless gene, and the inserted nucleic acid construct can be operably linked to an endogenous promoter in the target genomic locus (e.g., ALB locus). Use of an endogenous promoter is advantageous because it obviates the need for inclusion of a promoter in the nucleic acid construct, allowing packaging of larger transgenes that may not normally package efficiently (e.g., in AAV). Alternatively, the coding sequence for the polypeptide of interest in the nucleic acid construct can be operably linked to an exogenous promoter in the nucleic acid construct. Examples of types of promoters that can be used are disclosed elsewhere herein.

Optionally, some or all of the endogenous gene (e.g., endogenous ALB gene) at the target genomic locus can be expressed upon insertion of the multidomain therapeutic protein coding sequence from the nucleic acid construct. Alternatively, in some methods, none of the endogenous gene at the target genomic locus is expressed. As one example, the modified target genomic locus (e.g., modified ALB locus) after integration of the nucleic acid construct can encode a chimeric protein comprising an endogenous secretion signal (e.g., albumin secretion signal) and the polypeptide of interest encoded by the nucleic acid construct. In another example, the first intron of an ALB locus can be targeted. The secretion signal peptide of ALB is encoded by exon 1 of the ALB gene. In such a scenario, a promoterless cassette bearing a splice acceptor and the coding sequence for the polypeptide of interest will support expression and secretion of the polypeptide of interest. Splicing between endogenous ALB exon 1 and the integrated coding sequence for the polypeptide of interest creates a chimeric mRNA and protein including the endogenous ALB sequence encoded by exon 1 operably linked to the coding sequence for the polypeptide of interest encoded by the integrated nucleic acid construct.

The nucleic acid construct can be inserted into the target genomic locus by any means, including homologous recombination (HR) and non-homologous end joining (NHEJ) as described elsewhere herein. In a specific example, the nucleic acid construct is inserted by NHEJ (e.g., does not comprise a homology arm and is inserted by NHEJ).

In another specific example, the nucleic acid construct can be inserted via homology-independent targeted integration (e.g., directional homology-independent targeted integration). For example, the coding sequence for the polypeptide of interest in the nucleic acid construct can be flanked on each side by a target site for a nuclease agent (e.g., the same target site as in the target genomic locus, and the same nuclease agent being used to cleave the target site in the target genomic locus). The nuclease agent can then cleave the target sites flanking the coding sequence for the polypeptide of interest. In a specific example, the nucleic acid construct is delivered AAV-mediated delivery, and cleavage of the target sites flanking the coding sequence for the polypeptide of interest can remove the inverted terminal repeats (ITRs) of the AAV. Removal of the ITRs can make it easier to assess successful targeting, because presence of the ITRs can hamper sequencing efforts due to the repeated sequences. In some methods, the target site in the target genomic locus (e.g., a gRNA target sequence including the flanking protospacer adjacent motif) is no longer present if the coding sequence for the polypeptide of interest is inserted into the target genomic locus in the correct orientation but it is reformed if the coding sequence for the polypeptide of interest is inserted into the target genomic locus in the opposite orientation. This can help ensure that the coding sequence for the is inserted in the correct orientation for expression.

In any of the above methods, the B cell depleting agent can be administered simultaneously with the nucleic acid construct and/or nuclease agent (e.g., CRISPR/Cas system) or not simultaneously (e.g., sequentially in any combination). For example, in a method comprising administering a composition or combination comprising a B cell depleting agent, a nucleic acid construct, and a nuclease agent, they can be administered separately (e.g., the B cell depleting agent separately from the nucleic acid construct and/or nuclease agent). For example, the B cell depleting agent can be administered prior to the nucleic acid construct and/or nuclease agent, subsequent to the nucleic acid construct and/or nuclease agent, prior to and subsequent to the nucleic acid construct and/or nuclease agent, or at the same time as the nucleic acid construct and/or nuclease agent. Any suitable methods of administering B cell depleting agents, nucleic acid constructs, and nuclease agents to cells can be used (particularly methods of administering to the liver for the nucleic acid constructs and nuclease agents), and examples of such methods are described in more detail elsewhere herein.

In methods in which a composition or combination comprising a B cell depleting agent, a nucleic acid construct (or vector or LNP), and a nuclease agent is administered (i.e., in methods in which a B cell depleting agent, a nucleic acid construct (or vector or LNP), and a nuclease agent are both administered), the B cell depleting agent, the nucleic acid construct, and the nuclease agent can be administered simultaneously. Alternatively, the B cell depleting agent and the nucleic acid construct and the nuclease agent can be administered sequentially in any order. For example, the B cell depleting agent can be administered prior to and/or after the nucleic acid construct and/or nuclease agent. In one example, the B cell depleting agent can be administered prior to and after the nucleic acid construct and/or nuclease agent. In another example, the B cell depleting agent can be administered simultaneously with and after the nucleic acid construct and/or nuclease agent.

In one example, the B cell depleting agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the nucleic acid construct and/or nuclease agent.

In one example, the B cell depleting agent is administered about 4 hours, about 8 hours, about 12 hours, about 18 hours, about 1 day, about 2 days, about 3 days, about 4 days, about 5 days, about 6 days, or about 1 week prior to administering the nucleic acid construct and/or nuclease agent. In another example, the B cell depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nucleic acid construct and/or nuclease agent. In another example, the B cell depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nucleic acid construct and/or nuclease agent.

In one example, the B cell depleting agent is administered about 1 week prior to administering the nucleic acid construct and/or nuclease agent. In another example, the B cell depleting agent is administered within about 1 week prior to administering the nucleic acid construct and/or nuclease agent. In another example, the B cell depleting agent is administered at least about 1 day, at least about 2 days, at least about 3 days, at least about 4 days, at least about 5 days, at least about 6 days, or at least about 1 week prior to administering the nucleic acid construct and/or nuclease agent. In another example, the B cell depleting agent is administered about 1 day, about 2 days, about 3 days, about 4 days, about 5 days, about 6 days, or about 1 week prior to administering the nucleic acid construct and/or nuclease agent. In another example, the B cell depleting agent is administered about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, or about 6 days to about 7 days prior to administering the nucleic acid construct and/or nuclease agent.

In one example, the B cell depleting agent is administered about 4 hours, about 8 hours, about 12 hours, about 18 hours, about 1 day, about 2 days, about 3 days, about 4 days, about 5 days, about 6 days, or about 1 week prior to and subsequent to administering the nucleic acid construct and/or nuclease agent. In another example, the B cell depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering the nucleic acid construct and/or nuclease agent. In another example, the B cell depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the nucleic acid construct and/or nuclease agent.

In one example, the B cell depleting agent is administered about 4 hours, about 8 hours, about 12 hours, about 18 hours, about 1 day, about 2 days, about 3 days, about 4 days, about 5 days, about 6 days, or about 1 week after administering the nucleic acid construct and/or nuclease agent. In another example, the B cell depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nucleic acid construct and/or nuclease agent. In another example, the B cell depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nucleic acid construct and/or nuclease agent.

In any of the above methods, the nucleic acid construct can be administered simultaneously with the nuclease agent (e.g., CRISPR/Cas system) or not simultaneously (e.g., sequentially in any combination). For example, in a method comprising administering a composition comprising the nucleic acid construct and a nuclease agent, they can be administered separately. For example, the nucleic acid construct can be administered prior to the nuclease agent, subsequent to the nuclease agent, or at the same time as the nuclease agent. Any suitable methods of administering nucleic acid constructs and nuclease agents to cells can be used, particularly methods of administering to the liver, and examples of such methods are described in more detail elsewhere herein. In methods of treatment or in methods of targeting a cell in vivo in a subject, the nucleic acid construct can be inserted in particular types of cells in the subject. The method and vehicle for introducing nucleic acid construct and/or the nuclease agent into the subject can affect which types of cells in the subject are targeted. In some methods, for example, the nucleic acid construct is inserted into a target genomic locus (e.g., an endogenous ALB locus) in liver cells, such as hepatocytes. Methods and vehicles for introducing such constructs and nuclease agents into the subject (including methods and vehicles that target the liver or hepatocytes, such as lipid nanoparticle-mediated delivery and AAV-mediated delivery (e.g., rAAV8-mediated delivery) and intravenous injection), are disclosed in more detail elsewhere herein.

In methods in which a composition comprising a nucleic acid construct (or vector or LNP) and a nuclease agent is administered (i.e., in methods in which a nucleic acid construct (or vector or LNP) and a nuclease agent are both administered), the nucleic acid construct and the nuclease agent can be administered simultaneously. Alternatively, the nucleic acid construct and the nuclease agent can be administered sequentially in any order. For example, the nucleic acid construct can be administered after the nuclease agent, or the nuclease agent can be administered after the nucleic acid construct. For example, the nuclease agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to or subsequent to administration of the nucleic acid construct.

In one example, the nucleic acid construct is administered about 4 hours, about 8 hours, about 12 hours, about 18 hours, about 1 day, about 2 days, about 3 days, about 4 days, about 5 days, about 6 days, or about 1 week prior to administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to administering the nuclease agent.

In one example, the nucleic acid construct is administered about 4 hours, about 8 hours, about 12 hours, about 18 hours, about 1 day, about 2 days, about 3 days, about 4 days, about 5 days, about 6 days, or about 1 week after administering the nuclease agent. In another example, the nucleic acid construct is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the nuclease agent. In another example, the nucleic acid construct is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the nuclease agent.

In any of the above methods, the nucleic acid construct and the nuclease agent (e.g., CRISPR/Cas system) can be administered using any suitable delivery system and known method. The nuclease agent components and nucleic acid construct (e.g., the guide RNA, Cas protein, and nucleic acid construct) can be delivered individually or together in any combination, using the same or different delivery methods as appropriate.

In methods in which a CRISPR/Cas system is used, a guide RNA can be introduced into or administered to a subject or cell, for example, in the form of an RNA (e.g., in vitro transcribed RNA, such as the modified guide RNAs disclosed herein) or in the form of a DNA encoding the guide RNA. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be introduced into a subject or cell in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)), such as a modified mRNA as disclosed herein, or DNA). Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

In one example, the Cas protein is introduced in the form of an mRNA (e.g., a modified mRNA as disclosed herein), and the guide RNA is introduced in the form of RNA such as a modified gRNA as disclosed herein (e.g., together within the same lipid nanoparticle). Guide RNAs can be modified as disclosed elsewhere herein. Likewise, Cas mRNAs can be modified as disclosed elsewhere herein.

In methods in which a nucleic acid construct is inserted following cleavage by a gene-editing system (e.g., a Cas protein), the gene-editing system (e.g., Cas protein) can cleave the target genomic locus to create a single-strand break (nick) or double-strand break, and the cleaved or nicked locus can be repaired by insertion of the nucleic acid construct via non-homologous end joining (NHEJ)-mediated insertion or homology-directed repair. Optionally, repair with the nucleic acid construct removes or disrupts the guide RNA target sequence(s) so that alleles that have been targeted cannot be re-targeted by the CRISPR/Cas reagents.

As explained in more detail elsewhere herein, the nucleic acid constructs can comprise deoxyribonucleic acid (DNA) or ribonucleic acid (RNA), they can be single-stranded or double-stranded, and they can be in linear or circular form. The nucleic acid constructs can be naked nucleic acids or can be delivered by viruses, such as AAV. In a specific example, the nucleic acid construct can be delivered via AAV and can be capable of insertion into the target genomic locus (e.g., a safe harbor gene, an ALB gene, or intron 1 of an ALB gene) by non-homologous end joining (e.g., the nucleic acid construct can be one that does not comprise a homology arm).

Some nucleic acid constructs are capable of insertion by non-homologous end joining. In some cases, such nucleic acid constructs do not comprise a homology arm. For example, such nucleic acid constructs can be inserted into a blunt end double-strand break following cleavage with a Cas protein. In a specific example, the nucleic acid construct can be delivered via AAV and can be capable of insertion by non-homologous end joining (e.g., the nucleic acid construct can be one that does not comprise a homology arm).

In another example, the nucleic acid construct can be inserted via homology-independent targeted integration. For example, the nucleic acid construct can be flanked on each side by a guide RNA target sequence (e.g., the same target site as in the target genomic locus, and the CRISPR/Cas reagent (Cas protein and guide RNA) being used to cleave the target site in the target genomic locus). The Cas protein can then cleave the target sites flanking the nucleic acid insert. In a specific example, the nucleic acid construct is delivered AAV-mediated delivery, and cleavage of the target sites flanking the nucleic acid insert can remove the inverted terminal repeats (ITRs) of the AAV. In some methods, the target site in the target genomic locus (e.g., a guide RNA target sequence including the flanking protospacer adjacent motif) is no longer present if the nucleic acid insert is inserted into the target genomic locus in the correct orientation but it is reformed if the nucleic acid insert is inserted into the target genomic locus in the opposite orientation.

The methods disclosed herein can comprise introducing or administering into a subject (e.g., an animal or mammal, such as a human) or cell a nucleic acid construct and optionally a nuclease agent such as CRISPR/Cas reagents, including in the form of nucleic acids (e.g., DNA or RNA), proteins, or nucleic-acid-protein complexes. “Introducing” or “administering” includes presenting to the cell or subject the molecule(s) (e.g., nucleic acid(s) or protein(s)) in such a manner that it gains access to the interior of the cell or to the interior of cells within the subject. The introducing can be accomplished by any means, and two or more of the components (e.g., two of the components, or all of the components) can be introduced into the cell or subject simultaneously or sequentially in any combination. For example, a Cas protein can be introduced into a cell or subject before introduction of a guide RNA, or it can be introduced following introduction of the guide RNA. As another example, a multidomain therapeutic protein nucleic acid construct can be introduced prior to the introduction of a Cas protein and a guide RNA, or it can be introduced following introduction of the Cas protein and the guide RNA (e.g., the multidomain therapeutic protein nucleic acid construct can be administered about 1, 2, 3, 4, 8, 12, 24, 36, 48, or 72 hours before or after introduction of the Cas protein and the guide RNA). See, e.g., US 2015/0240263 and US 2015/0110762, each of which is herein incorporated by reference in its entirety for all purposes. In addition, two or more of the components can be introduced into the cell or subject by the same delivery method or different delivery methods. Similarly, two or more of the components can be introduced into a subject by the same route of administration or different routes of administration.

A guide RNA can be introduced into a subject or cell, for example, in the form of an RNA (e.g., in vitro transcribed RNA) or in the form of a DNA encoding the guide RNA. Guide RNAs can be modified as disclosed elsewhere herein. When introduced in the form of a DNA, the DNA encoding a guide RNA can be operably linked to a promoter active in the cell or in a cell in the subject. For example, a guide RNA may be delivered via AAV and expressed in vivo under a U6 promoter. Such DNAs can be in one or more expression constructs. For example, such expression constructs can be components of a single nucleic acid molecule. Alternatively, they can be separated in any combination among two or more nucleic acid molecules (i.e., DNAs encoding one or more CRISPR RNAs and DNAs encoding one or more tracrRNAs can be components of a separate nucleic acid molecules).

Likewise, Cas proteins can be provided in any form. For example, a Cas protein can be provided in the form of a protein, such as a Cas protein complexed with a gRNA. Alternatively, a Cas protein can be provided in the form of a nucleic acid encoding the Cas protein, such as an RNA (e.g., messenger RNA (mRNA)) or DNA. Cas RNAs can be modified as disclosed elsewhere herein. Optionally, the nucleic acid encoding the Cas protein can be codon optimized for efficient translation into protein in a particular cell or organism. For example, the nucleic acid encoding the Cas protein can be modified to substitute codons having a higher frequency of usage in a mammalian cell, a human cell, a rodent cell, a mouse cell, a rat cell, or any other host cell of interest, as compared to the naturally occurring polynucleotide sequence. When a nucleic acid encoding the Cas protein is introduced into a cell or a subject, the Cas protein can be transiently, conditionally, or constitutively expressed in the cell or in a cell in the subject.

Nucleic acids encoding Cas proteins or guide RNAs can be operably linked to a promoter in an expression construct. Expression constructs include any nucleic acid constructs capable of directing expression of a gene or other nucleic acid sequence of interest (e.g., a Cas gene) and which can transfer such a nucleic acid sequence of interest to a target cell. For example, the nucleic acid encoding the Cas protein can be in a vector comprising a DNA encoding one or more gRNAs. Alternatively, it can be in a vector or plasmid that is separate from the vector comprising the DNA encoding one or more gRNAs. Suitable promoters that can be used in an expression construct include promoters active, for example, in one or more of a eukaryotic cell, a human cell, a non-human cell, a mammalian cell, a non-human mammalian cell, a rodent cell, a mouse cell, a rat cell, a hamster cell, a pluripotent cell, an embryonic stem (ES) cell, an adult stem cell, a developmentally restricted progenitor cell, an induced pluripotent stem (iPS) cell, or a one-cell stage embryo. For example, a suitable promoter can be active in a liver cell such as a hepatocyte. Such promoters can be, for example, conditional promoters, inducible promoters, constitutive promoters, or tissue-specific promoters. Optionally, the promoter can be a bidirectional promoter driving expression of both a Cas protein in one direction and a guide RNA in the other direction. Such bidirectional promoters can consist of (1) a complete, conventional, unidirectional Pol III promoter that contains 3 external control elements: a distal sequence element (DSE), a proximal sequence element (PSE), and a TATA box; and (2) a second basic Pol III promoter that includes a PSE and a TATA box fused to the 5′ terminus of the DSE in reverse orientation. For example, in the H1 promoter, the DSE is adjacent to the PSE and the TATA box, and the promoter can be rendered bidirectional by creating a hybrid promoter in which transcription in the reverse direction is controlled by appending a PSE and TATA box derived from the U6 promoter. See, e.g., US 2016/0074535, herein incorporated by references in its entirety for all purposes. Use of a bidirectional promoter to express genes encoding a Cas protein and a guide RNA simultaneously allows for the generation of compact expression cassettes to facilitate delivery. In preferred embodiments, promotors are accepted by regulatory authorities for use in humans. In certain embodiments, promotors drive expression in a liver cell.

Molecules (e.g., Cas proteins or guide RNAs or nucleic acids encoding) introduced into the subject or cell can be provided in compositions comprising a carrier increasing the stability of the introduced molecules (e.g., prolonging the period under given conditions of storage (e.g., −20° C., 4° C., or ambient temperature) for which degradation products remain below a threshold, such below 0.5% by weight of the starting nucleic acid or protein; or increasing the stability in vivo). Non-limiting examples of such carriers include poly(lactic acid) (PLA) microspheres, poly(D,L-lactic-coglycolic-acid) (PLGA) microspheres, liposomes, micelles, inverse micelles, lipid cochleates, and lipid microtubules.

Various methods and compositions are provided herein to allow for introduction of molecule (e.g., a nucleic acid or protein) into a cell or subject. Methods for introducing molecules into various cell types are known and include, for example, stable transfection methods, transient transfection methods, and virus-mediated methods.

Transfection protocols as well as protocols for introducing molecules into cells may vary. Non-limiting transfection methods include chemical-based transfection methods using liposomes; nanoparticles; calcium phosphate (Graham et al. (1973) Virology 52 (2): 456-67, Bacchetti et al. (1977) Proc. Natl. Acad. Sci. U.S.A. 74 (4):1590-4, and Kriegler, M (1991). Transfer and Expression: A Laboratory Manual. New York: W. H. Freeman and Company. pp. 96-97); dendrimers; or cationic polymers such as DEAE-dextran or polyethylenimine. Non-chemical methods include electroporation, sonoporation, and optical transfection. Particle-based transfection includes the use of a gene gun, or magnet-assisted transfection (Bertram (2006) Current Pharmaceutical Biotechnology 7, 277-28). Viral methods can also be used for transfection.

Introduction of nucleic acids or proteins into a cell can also be mediated by electroporation, by intracytoplasmic injection, by viral infection, by adenovirus, by adeno-associated virus, by lentivirus, by retrovirus, by transfection, by lipid-mediated transfection, or by nucleofection. Nucleofection is an improved electroporation technology that enables nucleic acid substrates to be delivered not only to the cytoplasm but also through the nuclear membrane and into the nucleus. In addition, use of nucleofection in the methods disclosed herein typically requires much fewer cells than regular electroporation (e.g., only about 2 million compared with 7 million by regular electroporation). In one example, nucleofection is performed using the LONZA® NUCLEOFECTOR™ system.

Introduction of molecules (e.g., nucleic acids or proteins) into a cell (e.g., a zygote) can also be accomplished by microinjection. In zygotes (i.e., one-cell stage embryos), microinjection can be into the maternal and/or paternal pronucleus or into the cytoplasm. If the microinjection is into only one pronucleus, the paternal pronucleus is preferable due to its larger size. Microinjection of an mRNA is preferably into the cytoplasm (e.g., to deliver mRNA directly to the translation machinery), while microinjection of a Cas protein or a polynucleotide encoding a Cas protein or encoding an RNA is preferable into the nucleus/pronucleus. Alternatively, microinjection can be carried out by injection into both the nucleus/pronucleus and the cytoplasm: a needle can first be introduced into the nucleus/pronucleus and a first amount can be injected, and while removing the needle from the one-cell stage embryo a second amount can be injected into the cytoplasm. If a Cas protein is injected into the cytoplasm, the Cas protein preferably comprises a nuclear localization signal to ensure delivery to the nucleus/pronucleus. Methods for carrying out microinjection are well known. See, e.g., Nagy et al. (Nagy A, Gertsenstein M, Vintersten K, Behringer R., 2003, Manipulating the Mouse Embryo. Cold Spring Harbor, New York: Cold Spring Harbor Laboratory Press); see also Meyer et al. (2010) Proc. Natl. Acad. Sci. U.S.A. 107:15022-15026 and Meyer et al. (2012) Proc. Natl. Acad. Sci. U.S.A. 109:9354-9359, each of which is herein incorporated by reference in its entirety for all purposes.

Other methods for introducing molecules (e.g., nucleic acid or proteins) into a cell or subject can include, for example, vector delivery, particle-mediated delivery, exosome-mediated delivery, lipid-nanoparticle-mediated delivery, cell-penetrating-peptide-mediated delivery, or implantable-device-mediated delivery. As specific examples, a nucleic acid or protein can be introduced into a cell or subject in a carrier such as a poly(lactic acid) (PLA) microsphere, a poly(D,L-lactic-coglycolic-acid) (PLGA) microsphere, a liposome, a micelle, an inverse micelle, a lipid cochleate, or a lipid microtubule. Some specific examples of delivery to a subject include hydrodynamic delivery, virus-mediated delivery (e.g., adeno-associated virus (AAV)-mediated delivery), and lipid-nanoparticle-mediated delivery.

Introduction of nucleic acids and proteins into cells or subjects can be accomplished by hydrodynamic delivery (HDD). For gene delivery to parenchymal cells, only essential DNA sequences need to be injected via a selected blood vessel, eliminating safety concerns associated with current viral and synthetic vectors. When injected into the bloodstream, DNA is capable of reaching cells in the different tissues accessible to the blood. Hydrodynamic delivery employs the force generated by the rapid injection of a large volume of solution into the incompressible blood in the circulation to overcome the physical barriers of endothelium and cell membranes that prevent large and membrane-impermeable compounds from entering parenchymal cells. In addition to the delivery of DNA, this method is useful for the efficient intracellular delivery of RNA, proteins, and other small compounds in vivo. See, e.g., Bonamassa et al. (2011) Pharm. Res. 28(4):694-701, herein incorporated by reference in its entirety for all purposes.

Introduction of nucleic acids can also be accomplished by virus-mediated delivery, such as AAV-mediated delivery or lentivirus-mediated delivery. Other exemplary viruses/viral vectors include retroviruses, adenoviruses, vaccinia viruses, poxviruses, and herpes simplex viruses. The viruses can infect dividing cells, non-dividing cells, or both dividing and non-dividing cells. The viruses can integrate into the host genome or alternatively do not integrate into the host genome. Such viruses can also be engineered to have reduced immunity. The viruses can be replication-competent or can be replication-defective (e.g., defective in one or more genes necessary for additional rounds of virion replication and/or packaging). Viruses can cause transient expression or longer-lasting expression. Viral vector may be genetically modified from their wild type counterparts. For example, the viral vector may comprise an insertion, deletion, or substitution of one or more nucleotides to facilitate cloning or such that one or more properties of the vector is changed. Such properties may include packaging capacity, transduction efficiency, immunogenicity, genome integration, replication, transcription, and translation. In some examples, a portion of the viral genome may be deleted such that the virus is capable of packaging exogenous sequences having a larger size. In some examples, the viral vector may have an enhanced transduction efficiency. In some examples, the immune response induced by the virus in a host may be reduced. In some examples, viral genes (such as integrase) that promote integration of the viral sequence into a host genome may be mutated such that the virus becomes non-integrating. In some examples, the viral vector may be replication defective. In some examples, the viral vector may comprise exogenous transcriptional or translational control sequences to drive expression of coding sequences on the vector. In some examples, the virus may be helper-dependent. For example, the virus may need one or more helper virus to supply viral components (such as viral proteins) required to amplify and package the vectors into viral particles. In such a case, one or more helper components, including one or more vectors encoding the viral components, may be introduced into a host cell or population of host cells along with the vector system described herein. In other examples, the virus may be helper-free. For example, the virus may be capable of amplifying and packaging the vectors without a helper virus. In some examples, the vector system described herein may also encode the viral components required for virus amplification and packaging.

Exemplary viral titers (e.g., AAV titers) include about 10¹², about 10¹³, about 10¹⁴, about 10¹⁵, and about 10¹⁶ vector genomes (vg)/mL, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/mL. Other exemplary viral titers (e.g., AAV titers) include about 10¹², about 10¹³, about 10¹⁴, about 10¹⁵, and about 10¹⁶ vector genomes (vg)/kg of body weight, or between about 10¹² to about 10¹⁶, between about 10¹² to about 10¹⁵, between about 10¹² to about 10¹⁴, between about 10¹² to about 10¹³, between about 10¹³ to about 10¹⁶, between about 10¹⁴ to about 10¹⁶, between about 10¹⁵ to about 10¹⁶, or between about 10¹³ to about 10¹⁵ vg/kg of body weight. In one example, the viral titer is between about 10¹³ to about 10¹⁴ vg/mL or vg/kg. In another example, the viral titer is between about 10¹² to about 10¹³ vg/mL or vg/kg (e.g., between about 10¹² to about 10¹³ vg/kg). In another example, the viral titer is between about 10¹² to about 10¹⁴ vg/mL or vg/kg (e.g., between about 10¹² to about 10¹⁴ vg/kg). For example, the viral titer can be between about 1.5E12 to about 1.5E13 vg/kg, can be about 1.5E12 vg/kg, or can be about 1.5E13 vg/kg. AAVs for use in the methods are discussed in more detail elsewhere herein. In another example, the viral titer is about 1E12 to about 2E14 vg/kg (e.g., without plasma cell depletion and redosing). In another example, the viral titer is about 3E11 vg/kg to about 5E13 vg/kg (e.g., 2-3× lower with plasma cell depletion, due to 2-3 separate administrations with redosing). Use of the plasma cell depleting agents or combinations comprising plasma cell depleting agents allows redosing, permitting step-wise dosing with lower doses (e.g., step-wise dosing of 2-3 doses with each dose being 2-3× lower than a dose would be in a one-time administration (e.g., without plasma cell depletion). In another example, the viral titer is about 1E12 to about 2E14 vg/kg (e.g., without B cell depletion and redosing). In another example, the viral titer is about 3E11 vg/kg to about 5E13 vg/kg (e.g., 2-3× lower with B cell depletion, due to 2-3 separate administrations with redosing). Use of the B cell depleting agents allows redosing, permitting step-wise dosing with lower doses (e.g., step-wise dosing of 2-3 doses with each dose being 2-3× lower than a dose would be in a one-time administration (e.g., without B cell depletion).

Introduction of nucleic acids and proteins can also be accomplished by lipid nanoparticle (LNP)-mediated delivery. For example, LNP-mediated delivery can be used to deliver a combination of Cas mRNA and guide RNA or a combination of Cas protein and guide RNA. LNP-mediated delivery can be used to deliver a guide RNA in the form of RNA. In a specific example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. As another example, Cas mRNA modifications can include substitution with N1-methyl-pseudouridine (e.g., fully substituted with N1-methyl-pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity. Lipid formulations can protect biological molecules from degradation while improving their cellular uptake. Lipid nanoparticles are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such lipid nanoparticles can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as DSPC. In another example, the other components can comprise a helper lipid such as cholesterol, an optional neutral lipid such as DSPC, and a stealth lipid such as S010, S024, S027, S031, or S033.

The LNP may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes. In certain LNPs, the cargo can include a guide RNA or a nucleic acid encoding a guide RNA. In certain LNPs, the cargo can include an mRNA encoding a Cas nuclease, such as Cas9, and a guide RNA or a nucleic acid encoding a guide RNA. In certain LNPs, the cargo can include a multidomain therapeutic protein nucleic acid construct. In certain LNPs, the cargo can include an mRNA encoding a Cas nuclease, such as Cas9, a guide RNA or a nucleic acid encoding a guide RNA, and a multidomain therapeutic protein nucleic acid construct. LNPs for use in the methods are described in more detail elsewhere herein.

Exemplary dosing of LNPs includes about 0.1, about 0.25, about 0.3, about 0.5, about 1, about 2, about 3, about 4, about 5, about 6, about 8, or about 10 mg/kg body weight (mpk) or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 1, about 3, or about 10 mg/kg can be used. Additional exemplary dosing of LNPs includes about 0.1, about 0.25, about 0.3, about 0.5, about 1, about 2, about 3, about 4, about 5, about 6, about 8, or about 10 mg/kg (mpk) body weight or about 0.1 to about 10, about 0.25 to about 10, about 0.3 to about 10, about 0.5 to about 10, about 1 to about 10, about 2 to about 10, about 3 to about 10, about 4 to about 10, about 5 to about 10, about 6 to about 10, about 8 to about 10, about 0.1 to about 8, about 0.1 to about 6, about 0.1 to about 5, about 0.1 to about 4, about 0.1 to about 3, about 0.1 to about 2, about 0.1 to about 1, about 0.1 to about 0.5, about 0.1 to about 0.3, about 0.1 to about 0.25, about 0.25 to about 8, about 0.3 to about 6, about 0.5 to about 5, about 1 to about 5, or about 2 to about 3 mg/kg body weight with respect to total RNA (Cas9 mRNA and gRNA) cargo content. Such LNPs can be administered, for example, intravenously. In one example, LNP doses between about 0.01 mg/kg and about 10 mg/kg, between about 0.1 and about 10 mg/kg, or between about 0.01 and about 0.3 mg/kg can be used. For example, LNP doses of about 0.01, about 0.03, about 0.1, about 0.3, about 0.5, about 1, about 2, about 3, or about 10 mg/kg can be used. In another example, LNP doses between about 0.5 and about 10, between about 0.5 and about 5, between about 0.5 and about 3, between about 1 and about 10, between about 1 and about 5, between about 1 and about 3, or between about 1 and about 2 mg/kg can be used. In another example, LNP doses between about 0.5 and about 3, between about 0.5 and about 2.5, between about 0.5 and about 2, between about 0.5 and about 1.5, between about 0.5 and about 1, between about 1 and about 3, between about 1 and about 2.5, between about 1 and about 2, or between about 1 and about 1.5 mg/kg can be used. In another example, an LNP dose of about 1 mg/kg can be used.

The mode of delivery can be selected to decrease immunogenicity. For example, a Cas protein and a gRNA may be delivered by different modes (e.g., bi-modal delivery). These different modes may confer different pharmacodynamics or pharmacokinetic properties on the subject delivered molecule (e.g., Cas or nucleic acid encoding, gRNA or nucleic acid encoding, or multidomain therapeutic protein nucleic acid construct). For example, the different modes can result in different tissue distribution, different half-life, or different temporal distribution. Some modes of delivery (e.g., delivery of a nucleic acid vector that persists in a cell by autonomous replication or genomic integration) result in more persistent expression and presence of the molecule, whereas other modes of delivery are transient and less persistent (e.g., delivery of an RNA or a protein). Delivery of Cas proteins in a more transient manner, for example, as mRNA or protein, can ensure that the Cas/gRNA complex is only present and active for a short period of time and can reduce immunogenicity caused by peptides from the bacterially-derived Cas enzyme being displayed on the surface of the cell by MHC molecules. Such transient delivery can also reduce the possibility of off-target modifications.

Administration in vivo can be by any suitable route including, for example, parenteral, intravenous, oral, subcutaneous, intra-arterial, intracranial, intrathecal, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjuctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous.

Administration in vivo can be by any suitable route including, for example, parenteral, intravenous, oral, subcutaneous, intra-arterial, intracranial, intrathecal, intraperitoneal, topical, intranasal, or intramuscular. A specific example is intravenous infusion.

Administration in vivo can be by any suitable route including, for example, systemic routes of administration such as parenteral administration, e.g., intravenous, subcutaneous, intra-arterial, or intramuscular. In a specific example, administration in vivo is intravenous.

Compositions comprising the guide RNAs and/or Cas proteins (or nucleic acids encoding the guide RNAs and/or Cas proteins) can be formulated using one or more physiologically and pharmaceutically acceptable carriers, diluents, excipients or auxiliaries. The formulation can depend on the route of administration chosen. Pharmaceutically acceptable means that the carrier, diluent, excipient, or auxiliary is compatible with the other ingredients of the formulation and not substantially deleterious to the recipient thereof. In a specific example, the route of administration and/or formulation or chosen for delivery to the liver (e.g., hepatocytes).

The frequency of administration and the number of dosages can depend on a number of factors. The introduction of nucleic acids or proteins into the cell or subject can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. In some methods, a single administration of the nucleic acid construct (or a single administration of the nucleic acid construct and nuclease agent (e.g., Cas protein and guide RNA)) is sufficient to increase expression of polypeptide of interest to a desirable level. In other methods, more than one administration may be beneficial to maximize therapeutic effect.

The methods disclosed herein can increase polypeptide of interest protein levels and/or polypeptide of interest activity levels in a cell or subject (e.g., circulating, serum, or plasma levels in a subject) and can comprise measuring polypeptide of interest protein levels and/or polypeptide of interest activity levels in a cell or subject (e.g., circulating, serum, or plasma levels in a subject). In one example, the methods result in increased expression of the polypeptide of interest in the subject compared to a method comprising administering an episomal expression vector encoding the polypeptide of interest. For example, the methods can result in increased serum levels of the polypeptide of interest in the subject compared to a method comprising administering an episomal expression vector encoding the polypeptide of interest. The methods can also result in increased polypeptide of interest activity in the subject compared to a method comprising administering an episomal expression vector encoding the polypeptide of interest.

In some methods, polypeptide of interest activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, polypeptide of interest activity and/or expression levels in a subject are increased to about or at least about 40%, about or at least about 50%, about or at least about 75%, or at least about 100%, or more, of normal level.

In some methods, circulating polypeptide of interest levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, polypeptide of interest levels are at least about 1 μg/mL or about 1 μg/mL. In some methods, polypeptide of interest levels are at least about 2 μg/mL or about 2 μg/mL. In some methods, polypeptide of interest levels are at least about 5 μg/mL or about 5 μg/mL. In some methods, polypeptide of interest levels are about 1 μg/mL to about 30 μg/mL, about 2 μg/mL to about 30 μg/mL, about 3 μg/mL to about 30 μg/mL, about 4 μg/mL to about 30 μg/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in polypeptide of interest levels of about 2 μg/mL to about 30 μg/mL or 2 μg/mL to about 20 μg/mL. For example, the method can result in polypeptide of interest levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of polypeptide of interest over the subject's baseline expression and/or activity (i.e., expression and/or activity prior to administration). In some methods, the method increases expression and/or activity of polypeptide of interest over the subject's baseline expression and/or activity (i.e., expression and/or activity prior to administration. In some methods, polypeptide of interest activity and/or polypeptide of interest expression or serum levels in a subject are increased by about or at least about 10%, about or at least about 25%, about or at least about 50%, about or at least about 75%, or about or at least about 100%, or more, as compared to the subject's polypeptide of interest expression or serum levels and/or activity before administration (i.e., the subject's baseline levels). In certain embodiments, the loss of function is nearly complete such that a relative activity cannot be determined. In certain embodiments, the level of expression is sufficient to treat at least one sign or symptom resulting from the loss of function of the polypeptide of interest.

In some methods, the method increases expression and/or activity of the polypeptide of interest over the cell's baseline expression and/or activity (i.e., expression and/or activity prior to administration). In some methods, the method increases expression and/or activity of polypeptide of interest over the cell's baseline expression and/or activity (i.e., expression and/or activity prior to administration. In some methods, polypeptide of interest activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the polypeptide of interest activity and/or expression levels before administration (i.e., the subject's baseline levels). In certain embodiments, the polypeptide of interest loss of function is nearly complete such that a relative activity cannot be determined. In certain embodiments, the level of expression is sufficient to treat at least one sign or symptom resulting from the loss of function of the polypeptide of interest.

In a specific example, the polypeptide of interest activity levels in a subject are increased to no more than about 300%, no more than about 250%, no more than about 200%, or no more than about 150% of normal polypeptide of interest activity levels.

In a specific example, the polypeptide of interest activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels. In a specific example, the polypeptide of interest activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels.

In some methods, the method results in increased expression of the polypeptide of interest in the subject (e.g., neonatal subject) compared to a method comprising administering an episomal expression vector encoding the polypeptide of interest in a control subject. In some methods, the method results in increased serum levels of the polypeptide of interest in the subject (e.g., neonatal subject) compared to a method comprising administering an episomal expression vector encoding the polypeptide of interest to a control subject.

In some methods, the method increases expression or activity of the polypeptide of interest over the subject's (e.g., neonatal subject's) baseline expression or activity of the polypeptide of interest (i.e., any percent change in expression that is larger than typical error bars). In some methods, the method results in expression of the polypeptide of interest at a detectable level above zero, e.g., at a statistically significant level, a clinically relevant level.

Some methods comprise achieving a durable or sustained effect in a human, such as an at least at least 8 weeks, at least 24 weeks, for example, at least 1 year, or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased polypeptide of interest activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of polypeptide of interest in a human is achieved by at least 7 days, at least 14 days, or at least 28 days, optionally at least 56 days, at least 80 days, or at least 96 days. In additional methods, the method comprises maintaining polypeptide of interest activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the polypeptide of interest can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the polypeptide of interest can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the polypeptide of interest is maintained at a level higher than the expression or activity of the polypeptide of interest prior to treatment (i.e., the subject's baseline). In some methods, expression or activity of the polypeptide of interest is considered sustained if it is maintained at a therapeutically effective level of expression or activity. Relative durations, in other organisms, are understood based, e.g., on life span and developmental stages, are covered within the disclosure above. In some methods, expression or activity of the polypeptide of interest is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., at 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., at 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., at 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the polypeptide of interest is sustained when the neonatal subject becomes an adult. In some methods, the expression of the polypeptide of interest is sustained for the lifetime of the subject or neonatal subject.

In some methods, the expression or activity of the polypeptide of interest is at least 50% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at one year after the administering. In some methods, the expression or activity of the polypeptide of interest is at least 60% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at two years after the administering. In some methods, the expression or activity of the polypeptide of interest is at least 60% of the expression or activity of the polypeptide of interest at a peak level of expression measured for the subject at 2 years after the administering. In some methods, the expression or activity of the polypeptide of interest is at least 60% of the expression or activity of the polypeptide of interest at a peak level of expression measured for the subject at 24 weeks after the administering.

In some methods involving insertion into an ALB locus, the subject's circulating albumin levels or cell's albumin levels are normal. Such methods may comprise maintaining the subject's circulating albumin levels or the cell's albumin levels within ±5%, ±10%, ±15%, ±20%, or ±50% of normal circulating albumin levels or normal albumin levels. In some methods, the subject's or cell's albumin levels are unchanged as compared to the albumin levels of untreated individuals by at least week 4, at least week 8, at least week 12, or at least week 20. In some methods, the subject's or cell's albumin levels transiently drop and then return to normal levels. In particular, the methods may comprise detecting no significant alterations in levels of plasma albumin.

In some methods, the method further comprises determining whether the subject has immunity against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent prior to the administering of any of the above. For example, the determining can comprise determining the presence of neutralizing antibodies against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent (e.g., determining the presence of neutralizing antibodies against an AAV comprising a nucleic acid construct). For example, the determining the presence of neutralizing antibodies can comprise determining whether there is an effective level of neutralizing antibody to prevent the intended outcome of insertion of a nucleic acid construct into a genomic locus or expression of the polypeptide of interest encoded by the nucleic acid construct. In some methods, the method further comprises assessing preexisting anti-polypeptide of interest immunity in a subject prior to administering any of the nucleic acid constructs described herein. For example, such methods could comprise assessing immunogenicity using a total antibody (TAb) immune assay or a neutralizing antibody (NAb) assay. In some methods, the subject has not previously been administered recombinant polypeptide of interest protein. In some methods, the subject has previously been administered recombinant polypeptide of interest protein.

In some methods, the method further comprises assessing preexisting anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase). In some embodiments, the subject does not have preexisting anti-AAV immunity. In some embodiments, the subject does have preexisting AAV immunity.

In various aspects, the present disclosure provides methods for inhibiting or preventing an immune response to an immunogen (e.g., an immunogenic delivery vehicle such as, e.g., AAV) in a subject in need thereof, the methods comprising administering to the subject an effective amount of a plasma cell depleting agent, a B cell depleting agent, and/or an immunoglobulin depleting agent disclosed herein. In some embodiments, the present disclosure provides methods for inhibiting or preventing generation of antibodies (e.g., neutralizing antibodies) to an immunogen in a subject in need thereof, the methods comprising administering to the subject an effective amount of a plasma cell depleting agent, a B cell depleting agent, and/or an immunoglobulin depleting agent. In some embodiments of methods of the disclosure comprising administering to the subject an effective amount of a plasma cell depleting agent and an immunogen (e.g., a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein, such as AAV), the subject has a pre-existing immunity against the immunogen (e.g., AAV). In another aspect, provided herein is a method for inhibiting generation of neutralizing antibodies to an immunogen in a subject in need thereof (e.g., a subject without a pre-existing immunity against the immunogen), the method comprising administering to the subject an effective amount of an anti-CD20×CD3 bispecific antibody or functional fragment thereof. In some embodiments, the present disclosure provides methods for increasing effectiveness of re-administration of an immunogen to a subject in need thereof, the methods comprising administering to the subject an effective amount of a plasma cell depleting agent, a B cell depleting agent, and/or an immunoglobulin depleting agent. In another aspect, provided herein is a method for increasing effectiveness of re-administration of an immunogen to a subject in need thereof (e.g., a subject without a pre-existing immunity against the immunogen), the method comprising administering to the subject an effective amount of an anti-CD20×CD3 bispecific antibody or functional fragment thereof. The term “re-administering” is used synonymously and interchangeably with the term “re-dosing” herein. In some embodiments, the present disclosure provides methods for increasing or maintaining the level of a transgene expression in a subject in need thereof, and the transgene is delivered to the subject via an immunogenic delivery vehicle (e.g., an AAV), the methods comprising administering to the subject an effective amount of a plasma cell depleting agent, a B cell depleting agent, and/or an immunoglobulin depleting agent.

In some embodiments, a plasma cell depleting agent, a B cell depleting agent, and/or an immunoglobulin depleting agent which can be useful in any of the methods or compositions disclosed herein may be further used in combination with a plasmapheresis, therapeutic plasma exchange, and/or immunoadsorption.

In some embodiments, administration of a plasma cell depleting agent, a B cell depleting agent, an immunoglobulin depleting agent, and/or an immunogen prevents or delays the increase of disease symptoms or the progression of disease in a subject having disease or condition.

In some embodiments, a plasma cell depleting agent, a B cell depleting agent, and/or an immunoglobulin depleting agent may inhibit an immune response by a cell (e.g., an immune cell such as by a B cell or a T cell) or by an immune system of a subject (e.g., a human) which can be elicited by an immunogen.

In some embodiments, a plasma cell depleting agent, a B cell depleting agent, and/or an immunoglobulin depleting agent may inhibit an immune response by a cell (e.g., an immune cell such as a B cell or a T cell) or by an immune system of a subject (e.g., a human) which can be elicited by an immunogenic delivery vehicle.

In one aspect, the present disclosure provides a method for increasing or maintaining the level of AAV transduction in a target cell and/or tissue, e.g., a target cell and/or tissue within or derived from a subject in need thereof, the method comprising contacting the target cell and/or tissue with, and/or administering to the subject, an effective amount of a plasma cell depleting agent, a B cell depleting agent, and/or an immunoglobulin depleting agent.

In another aspect, provided herein is a method for increasing or maintaining the level of AAV transduction in a target cell and/or tissue, e.g., a target cell and/or tissue within or derived from a subject in need thereof (e.g., a subject without pre-existing immunity against AAV), the method comprising contacting the target cell and/or tissue with, and/or administering to the subject, an effective amount of an anti-CD20×CD3 bispecific antibody or a functional fragment thereof. In some embodiments, the level of AAV transduction in the target cell and/or tissue is increased or maintained by inhibiting or preventing an immune response to the AAV in the subject. In some embodiments, the level of AAV transduction is increased or maintained in the target cell and/or tissue by inhibiting antibody responses to the AAV in the subject.

In some embodiments, the level of AAV transduction in the target cell and/or tissue is increased or maintained by inhibiting or preventing an immune response to the AAV in a subject. As a non-limiting example, the level of AAV transduction in the target cell and/or tissue may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of AAV transduction may be increased in the target cell and/or tissue by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level AAV transduction may be increased in the target cell and/or tissue by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, the level of AAV transduction in the target cell and/or tissue is maintained by inhibiting or preventing an immune response to the AAV in the subject.

In some embodiments, the level of AAV transduction in the target cell and/or tissue is increased or maintained by inhibiting antibody responses to the AAV in a subject. As a non-limiting example, the level of AAV transduction in the target cell and/or tissue may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of AAV transduction may be increased in the target cell and/or tissue by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level AAV transduction may be increased in the target cell and/or tissue by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%. In some embodiments, the level of AAV transduction in the target cell and/or tissue is maintained by inhibiting antibody responses to the AAV in the subject.

In one aspect, the present disclosure provides a method for increasing or maintaining the level of expression of a polypeptide of interest (e.g., expressed from an administered nucleic acid construct as described elsewhere herein) in a subject in need thereof, the method comprising administering to the subject an effective amount of a plasma cell depleting agent, a B cell depleting agent, and/or an immunoglobulin depleting agent. In some embodiments, the nucleic acid construct is delivered to the subject via an immunogenic delivery vehicle (e.g., AAV). In another aspect, provided herein is a method for increasing or maintaining the level of expression of a polypeptide of interest (e.g., expressed from an administered nucleic acid construct as described elsewhere herein) in a subject in need thereof, the method comprising administering to the subject an effective amount of an anti-CD20×CD3 bispecific antibody or a functional fragment thereof. In some embodiments, the nucleic acid construct is delivered to the subject via an immunogenic delivery vehicle (e.g., AAV). In some embodiments, the subject does not have a pre-existing immunity against the immunogenic delivery vehicle and/or transgene product(s) (e.g., does not have pre-existing immunity against a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein, such as AAV).

In some embodiments, the level of expression of a polypeptide of interest is increased or maintained by inhibiting an immune response to the immunogenic delivery vehicle and/or by inhibiting an immune response to a polypeptide or polynucleotide encoded by the nucleic acid construct. In some embodiments, the level of expression of a polypeptide of interest is increased or maintained by inhibiting antibody responses to the polypeptide or polynucleotide encoded by the nucleic acid construct.

In some embodiments, the level of expression of a polypeptide of interest is increased or maintained by inhibiting an immune response to the immunogenic delivery vehicle and/or by inhibiting an immune response to the transgene product(s). In some embodiments, the level of expression of a polypeptide of interest is increased or maintained by inhibiting antibody responses to the transgene product(s).

In some embodiments, the level of expression of the polypeptide of interest is increased or maintained by inhibiting or preventing an immune response to the immunogenic delivery vehicle and/or by inhibiting or preventing an immune response to one or more other delivered components (e.g., nucleic acid construct, nuclease agent or CRISPR/Cas system). As a non-limiting example, the level of expression of the polypeptide of interest may be increased by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The level of expression of the polypeptide of interest may be increased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The level of expression of the polypeptide of interest may be increased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the immune response to the immunogenic delivery vehicle and/or the immune response to one or more other delivered components (e.g., nucleic acid construct, nuclease agent or CRISPR/Cas system) may be inhibited by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The immune response to the immunogenic delivery vehicle and/or the immune response to one or more other delivered components may be inhibited by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The immune response to the immunogenic delivery vehicle and/or the immune response to one or more other delivered components may be inhibited by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In another aspect, provided herein is a method for inhibiting or preventing an immune response to an immunogen in a subject in need thereof (e.g., a subject without a pre-existing immunity against the immunogen, such as a nucleic acid construct described herein, a polypeptide of interest encoded by a nucleic acid construct described herein, a nuclease agent or one or more nucleic acids encoding the nuclease agent as described herein, or a delivery vehicle for the nucleic acid construct, the nuclease agent, or the one or more nucleic acids encoding the nuclease agent as described herein, such as AAV), the method comprising administering to the subject an effective amount of an anti-CD20×CD3 bispecific antibody or a functional fragment thereof.

In some embodiments, inhibiting or preventing the immune response comprises suppression of numbers and frequencies of immunogen-specific B cells.

In some embodiments of the methods for inhibiting or preventing an immune response to an immunogen described herein, the inhibiting of the immune response can comprise suppression of numbers and/or frequencies of plasma cells and/or B cells.

In some embodiments, the number and/or frequency of plasma cells and/or B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The number and/or frequency of plasma cells and/or B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The number and/or frequency of plasma cells and/or B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the total number and/or frequency of plasma cells and/or B cells may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The total number and/or frequency of plasma cells and/or B cells may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The total number and/or frequency of plasma cells and/or B cells may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, inhibiting the immune response comprises suppression of immunogen-specific IgG and/or IgM responses.

In some embodiments, the responses of IgG and/or IgM may be reduced by about 1%, about 2%, about 3%, about 4%, about 5%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The responses of IgG may be reduced by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The responses of IgG may be reduced by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

In some embodiments, the effectiveness of re-administration of an immunogen may be increased by about 1%, about 2%, about 3%, about 4%, about 5%, about 6%, about 7% about 8%, about 9%, about 10%, from about 10% to about 15%, from about 15% to about 20%, from about 20% to about 25%, from about 25% to about 30%, from about 30% to about 40%, from about 40% to about 50% or more. The effectiveness of re-administration of an immunogen be increased by from about 50% to about 60%, from about 50% to about 70%, from about 50% to about 80%, from about 50% to about 90%, more than 60%, from about 60% to about 70%, from about 60% to about 80%, from about 60% to about 90%, more than about 70%, from about 70% to about 80%, from about 70% to about 90%, more than about 80%, from about 80% to about 90%, more than 90%, from about 90% to about 95%, from about 90% to about 98%, more than 95%, from about 95% to about 98%, more than about 98%, or more than about 99%. The effectiveness of re-administration of an immunogen may be increased by about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or even 100%.

### A. Hemophilia B

In some methods disclosed herein, the polypeptide of interest is a Factor IX (FIX) protein disclosed herein, and the enzyme deficiency is FIX deficiency or hemophilia B. See, e.g., WO 2023/077012 and US 2023-0149563, each of which is herein incorporated by reference in its entirety for all purposes. In such methods, the nucleic acid constructs and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of reducing glycogen accumulation in a cell or a population of cells or a tissue in a subject, methods of treating hemophilia B disease or FIX deficiency in a subject, and methods or preventing or reducing the onset of a sign or symptom of hemophilia B or FIX deficiency in a subject.

The compositions disclosed herein (e.g., F9 nucleic acid constructs, or F9 nucleic acid constructs in combination with plasma cell depleting agents or combinations comprising plasma cell depleting agents and the nuclease agents (e.g., CRISPR/Cas systems)) are useful for the treatment of FIX deficiency or hemophilia B and/or ameliorating at least one symptom associated with FIX deficiency or hemophilia B. Likewise, the compositions disclosed herein can be used for the preparation of a pharmaceutical composition or medicament for treating a subject having FIX deficiency or hemophilia B.

The compositions disclosed herein (e.g., F9 nucleic acid constructs, or F9 nucleic acid constructs in combination with B cell depleting agents (e.g., anti-CD20×CD3 antigen-binding molecules) and the nuclease agents (e.g., CRISPR/Cas systems)) are useful for the treatment of FIX deficiency or hemophilia B and/or ameliorating at least one symptom associated with FIX deficiency or hemophilia B. Likewise, the compositions disclosed herein can be used for the preparation of a pharmaceutical composition or medicament for treating a subject having FIX deficiency or hemophilia B.

Symptoms and Cause. Hemostasis is a balance between procoagulant and anticoagulant activity to maintain blood in a fluid state under normal conditions and rapidly form a blood clot in the case of a vessel injury. Coagulation is initiated by disruption of the endothelium exposing platelets to collagen and extravascular tissue factor. This results in an activation cascade of clotting factors, including FIX, amplifying the coagulation reaction until activation and polymerization of fibrin monomers form a plug to block blood flow and achieve hemostasis. The coagulation process is accompanied by clot containment, wound healing, clot dissolution, tissue regeneration and remodeling.

Hemophilia B is a rare congenital disorder caused by an inherited or spontaneous recessive mutation in the F9 gene present on the X chromosome, leading to the expression of a malfunctioning or deficient FIX protein. The absence of a functioning FIX protein interrupts the coagulation cascade and greatly limits the normal formation of blood clots, leading to abnormal bleeding. Due to the recessive nature of the variant, hemophilia usually only affects men, although female carriers may experience mild to moderate symptoms and may require treatment.

Hemophilia is usually inherited from the maternal X chromosome, but prospective studies report that 43% of people newly diagnosed with severe hemophilia B have no prior family history of hemophilia. See, e.g., Srivastava et al. (2020) Haemophilia 26.6:1-158 and Kasper et al. (2007) Haemophilia 13:90-92, each of which is herein incorporated by reference in its entirety for all purposes. The report on the Annual Global Survey conducted in 2019 by the World Federation of Hemophilia reported that 31,997 patients lived with Hemophilia B in the world, with 4,093 patients living in the U.S. See, e.g., World Federation of Hemophilia, “Report on the Annual Global Survey 2019” World Federation of Hemophilia (2020), herein incorporated by reference in its entirety for all purposes. The estimated incidence (prevalence at birth) of hemophilia B worldwide is 5.0 cases per 100,000 males, and 1.5 cases per 100,000 males for severe hemophilia B. Due to the higher mortality rate for people with hemophilia, the estimated prevalence across age groups is lower, with 3.8 cases of hemophilia B per 100,000 males, including 1.1 cases of severe hemophilia per 100,000 males. See, e.g., World Federation of Hemophilia, “Report on the Annual Global Survey 2019” World Federation of Hemophilia (2020), herein incorporated by reference in its entirety for all purposes.

The severity of hemophilia and bleeding manifestations correlates with the degree of clotting factor's activity levels (Table 11). See, e.g., Srivastava et al. (2020) Haemophilia 26.6:1-158, herein incorporated by reference in its entirety for all purposes. In high-income markets, it is estimated that 34% of hemophilia B patients have mild hemophilia, around 31% have a moderate hemophilia, and 33% have a severe hemophilia. See, e.g., World Federation of Hemophilia, “Report on the Annual Global Survey 2019” World Federation of Hemophilia (2020), herein incorporated by reference in its entirety for all purposes.

TABLE 11
Severity Classification of Hemophilia B Based on Clotting Factor Activity Level.

| Severity | FIX Activity Levels | Clinical Symptoms |
|---|---|---|
| Mild | 6-49% of normal, | Typically experience bleeding only after serious injury, trauma, or surgery |
| | 0.06-0.40 IU/mL | May not be diagnosed until well into adulthood. |
| | | Spontaneous bleeding rare, but may occur in patients with less than ~15- |
| | | 30% of normal FIX activity levels. |
| Moderate | 1-5% of normal, | Bleed infrequently, and experience prolonged bleeding following minor |
| | 0.01-0.05 IU/mL | surgery or injury. |
| | | Spontaneous bleeds may occur, generally <1 times per month. |
| Severe | <1% of normal, <0.01 | Experience bleeding after injury and may have frequent spontaneous |
| | IU/mL | bleeds several times per month, including in their joints and muscles. |

Clinical symptoms of hemophilia B include the observation of easy bruising, “spontaneous” bleeding in the joints, muscles and soft tissues, pain, excessive bleeding following trauma or surgery, and life-threatening intracranial bleeding.

Spontaneous bleeding most commonly occurs in the joints, with the knees (>50% of all bleeding events), elbows, ankles, shoulders, and wrists the most affected. The recurrence of the bleeds in the joints results in inflammation with swelling of the joint, degeneration of the cartilage and progressive destruction of the joint space called hemophilic arthropathy. As the arthropathy develops in a joint, bleedings become more and more frequent even when minimal joint stress is applied such as normal weight bearing, leading to chronic synovitis, pain, fibrosis and progressive joint stiffness. In the last phase of the hemophilic arthropathy, progressive and erosive destruction of the cartilage narrows the joint space leading to collapse or sclerosis of the joint.

Intramuscular hemorrhages represent 30% of bleeding events. When localized in confined spaces like fascial muscles, it may lead to significant compression of vital structures with ischemia, gangrene, contractures and neuropathy. Bleeding in the pelvic space might lead to femoral nerve compression leading to potential permanent disability if neuropathy develops. See, e.g., Napolitano et al., “Chapter 3—Hemophilia A and Hemophilia B,” Consultative Hemostasis and Thrombosis, Elsevier 4(2019):39-58, herein incorporated by reference in its entirety for all purposes.

Epistaxis, oral and gastrointestinal bleeding can also occur following minor trauma like coughing or vomiting. Bleedings in the abdominal wall or in the bowel wall can also occur producing severe pain often misdiagnosed for appendicitis for example. See, e.g., Hoots et al., “Clinical manifestations and diagnosis of hemophilia” UptoDate (2019), herein incorporated by reference in its entirety for all purposes. Hematuria is a frequent manifestation of severe hemophilia but is usually benign and not associated with progressive loss of renal function. See, e.g., Hoots et al., “Clinical manifestations and diagnosis of hemophilia” UptoDate (2019), herein incorporated by reference in its entirety for all purposes.

Intracranial hemorrhage is estimated to occur in approximately 2.7% of individuals with hemophilia and is spontaneous in 50% of the time in affected adults. Despite its low incidence, intracranial hemorrhage is the most common cause of death from bleeding in hemophilic patients. See, e.g., Napolitano et al., “Chapter 3—Hemophilia A and Hemophilia B,” Consultative Hemostasis and Thrombosis, Elsevier 4(2019):39-58, herein incorporated by reference in its entirety for all purposes.

The overall life expectancy of patients with hemophilia B varies whether patients receive appropriate treatment. With prophylaxis and on-demand treatment such as the ones available in the U.S., the median life expectancy was found to be 6 years less than healthy men (77 years old versus 83 years old) in a study conducted in The Netherlands in 2018. See, e.g., Hassan et al. (2021) J. Thromb. Haemost. 19:645-653, herein incorporated by reference in its entirety for all purposes.

Diagnostic. Patients with severe hemophilia B are usually diagnosed before the age of 2 years based on clinical features, whilst patients with mild hemophilia might be diagnosed at an older age if the bleeding symptoms only manifest at time of injuries or surgeries. See, e.g., Berntorp et al. (2021) Nat. Rev. Dis. Primers 7.45, herein incorporated by reference in its entirety for all purposes.

The diagnosis of general hemophilia is firstly based on the clinical features and is then confirmed by screening test results with a normal platelet count, a normal prothrombin time (PT) assay but a prolonged activated partial thromboplastin time (APTT). See, e.g., Srivastava et al. (2020) Haemophilia 26.6:1-158 and Hoots et al., “Clinical manifestations and diagnosis of hemophilia” UptoDate (2019), each of which is herein incorporated by reference in its entirety for all purposes. The final diagnosis of hemophilia B is based on the results of the one-stage FIX assay measuring FIX activity levels below 40% of normal. See, e.g., Srivastava et al. (2020) Haemophilia 26.6:1-158, herein incorporated by reference in its entirety for all purposes. In rare circumstances, the FIX activity level of hemophilia B patients may be ≥40%, and a pathogenic FIX level can be investigated.

A genetic diagnosis can be performed to define the disease biology, establish the diagnosis in difficult cases, predict risk of inhibitor development, identify female carriers, and provide prenatal diagnosis if desired. See, e.g., Srivastava et al. (2020) Haemophilia 26.6:1-158, herein incorporated by reference in its entirety for all purposes. Although it is recognized that genetic testing might not always identify the exact mutation, it is estimated that mutation responsible for hemophilia B is identified in the F9 gene in 98% of cases. See, e.g., Carcao et al., “Hemophilia A and B” Hematology, Elsevier 7(2018):2001-2022, herein incorporated by reference in its entirety for all purposes. Newborns can be tested for hemophilia B through a blood test to measure factor IX levels. For example, the blood can be drawn from the umbilical cord.

Treatment and Unmet Medical Need. The main objective of hemophilia B treatment is to prevent or treat bleedings by replacing the missing blood clotting factor to hemostatically adequate plasma levels for prevention or treatment of acute bleedings. See, e.g., Napolitano et al., “Chapter 3—Hemophilia A and Hemophilia B,” Consultative Hemostasis and Thrombosis, Elsevier 4(2019):39-58, herein incorporated by reference in its entirety for all purposes.

The current standard of care is the use of plasma-derived or recombinant FIX clotting factor concentrates (CFC) for the prevention or treatment of hemophilia B. See, e.g., Srivastava et al. (2020) Haemophilia 26.6:1-158, herein incorporated by reference in its entirety for all purposes. Prophylaxis by regular administration of CFC is recommended to prevent spontaneous bleeding in patients with moderate-to-severe hemophilia B to maintain FIX levels over 1%. FIX activity levels of 30-50% of normal are required to control minor or moderate bleedings or to prevent recurrent spontaneous bleedings (Srivastava et al. (2020) Haemophilia 26.6:1-158), representing 2 to 3 intravenous infusions of standard FIX CFC per week made under the supervision of a physician specialized in hemophilia treatment. See, e.g., Srivastava et al. (2020) Haemophilia 26.6:1-158 and Powell et al. (2013) N. Engl. J. Med. 369.24:2313-2323, each of which is herein incorporated by reference in its entirety for all purposes. Extended half-life CFCs have been developed to reduce the frequency of administration to every week or every two weeks. See, e.g., Powell et al. (2013) N. Engl. J. Med. 369.24:2313-2323, each of which is herein incorporated by reference in its entirety for all purposes. Initiation of prophylaxis is recommended as early as possible, and preferably before 3 years old, since age at initiation has been demonstrated to be a strong predictor of long-term clinical outcomes. See, e.g., Srivastava et al. (2020) Haemophilia 26.6:1-158, herein incorporated by reference in its entirety for all purposes. Gene therapy for hemophilia patients during the neonatal and infant stages can prevent irreversible symptoms and life-threatening events, such as hemophilic arthropathy and intracranial bleeding.

Episodic, or on-demand therapy, can be added to the prophylaxis in case of hemorrhage or surgical procedures in which case levels of 50-100% of normal FIX activity should be achieved and maintained for a minimum of 7 to 10 days. See, e.g., Carcao et al., “Hemophilia A and B” Hematology, Elsevier 7(2018):2001-2022, and Napolitano et al., “Chapter 3—Hemophilia A and Hemophilia B,” Consultative Hemostasis and Thrombosis, Elsevier 4(2019):39-58, each of which is herein incorporated by reference in its entirety for all purposes. On-demand therapy can be the only treatment for patients with moderate forms of hemophilia B who do not experience spontaneous bleeding. See, e.g., Srivastava et al. (2020) Haemophilia 26.6:1-158, herein incorporated by reference in its entirety for all purposes.

Patients with high adherence to prophylaxis treatment have been observed to have lower levels of annualized bleeding rate (ABR). Recent studies on the efficacy of rCFCs observed that the mean ABR across studies ranged between 0-4. See, e.g., Davis et al. (2019) J. Med. Econ. 22.10:1014-1021 and Chhabra (2020) Blood Coagul. Fibrinolysis 31.3:186-192, each of which is herein incorporated by reference in its entirety for all purposes.

In addition to pharmacological treatments, a multi-disciplinary team supporting the patient's care and education relating to hemophilia will be put in place, generally consisting of at least a hematologist, a nurse, and a physical therapist.

Although the use of CFCs in the U.S. has changed the overall course of the disease, there remains an unmet medical need for hemophilia B patients. Firstly, the development of neutralizing antibodies to exogenous FIX administered from the CFCs has been reported to occur in approximately 10% of individuals with severe FIX deficiency which can greatly interfere with the ability to treat bleedings. See, e.g., Male et al. (2021) Hemophilia 106.1:123-129, herein incorporated by reference in its entirety for all purposes. Secondly, prophylaxis treatment requiring several injections per week or an injection every 2 weeks at a dedicated center represents a significant treatment burden on the patient and a notable economic burden to healthcare systems and society. See, e.g., Burke et al. (2021) Orphanet. J. Rare Dis. 16:143, herein incorporated by reference in its entirety for all purposes. Even though prophylaxis drives the risk of spontaneous joint bleeding down, it does not completely eliminate it, leading to remaining chronic pain and disability. See, e.g., Burke et al. (2021) Orphanet. J. Rare Dis. 16:143, herein incorporated by reference in its entirety for all purposes. A treatment that would lead to sustained plasma levels of functional FIX protein on the long-term is thus still needed.

Adeno-associated viral (AAV) vector gene therapies are under investigation for hemophilia B. Clinical trial data demonstrates sustained endogenous production of FIX in some patients which results in the elimination of the need for infusion of replacement FIX. See, e.g., Von Drygalski (2020) Blood 136(supp 1):13, herein incorporated by reference in its entirety for all purposes. However, AAV expression of FIX is episomal, therefore durability of expression remains unknown until further long-term data on efficacy are generated.

Several recombinant adeno-associated viral (rAAV) vector episomal gene therapies are under Phase 3 investigation for hemophilia B. These rAAV vectors deliver codon-optimized Padua variant human F9 gene associated with a liver-specific promoter to the nuclei of hepatocytes where it will be maintained as an extrachromosomal circular episome. The resulting expressed Padua FIX protein is a variant of the wild type FIX protein which is estimated to have an eight-fold increase in FIX specific activity compared to the wild type FIX. See, e.g., VandenDriessche et al. (2018) Mol. Ther. 26.1:14-16, herein incorporated by reference in its entirety for all purposes. However, AAV expression of FIX is episomal, therefore durability of expression remains unknown until further long-term data on efficacy are generated.

Methods. Provided are methods of treating a FIX deficiency in a subject and methods of treating hemophilia B in a subject and methods of preventing or inhibiting spontaneous bleeding in a subject having hemophilia B. The hemophilia B can be any type of hemophilia B (e.g., mild hemophilia B, moderate hemophilia B, or severe hemophilia B). Hemophilia B is described in more detail elsewhere herein.

Such methods can comprise administering any of the F9 nucleic acid constructs described herein (or any of the compositions comprising a F9 nucleic acid construct described herein, including, for example, vectors or lipid nanoparticles) to the subject in combination with a plasma cell depleting agent or combination comprising the plasma cell depleting agent such that a therapeutically effective level of FIX expression is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the F9 nucleic acid construct or composition comprising the F9 nucleic acid construct can be administered without a nuclease agent (e.g., if the F9 nucleic acid construct comprises elements needed for expression of FIX without integration into a target genomic locus). In some methods, the F9 nucleic acid construct can be administered together with a nuclease agent described herein. The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target gene to create a cleavage site, the F9 nucleic acid construct can be inserted into the cleavage site to create a modified target gene, and FIX protein can be expressed from the modified target gene such that a therapeutically effective level of FIX expression is achieved in the subject. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene such that a therapeutically effective level of FIX expression is achieved in the subject.

Such methods can comprise administering any of the F9 nucleic acid constructs described herein (or any of the compositions comprising a F9 nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with a B cell depleting agent (e.g., anti-CD20×CD3 antigen-binding molecule) such that a therapeutically effective level of FIX expression is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the F9 nucleic acid construct or composition comprising the F9 nucleic acid construct can be administered without a nuclease agent (e.g., if the F9 nucleic acid construct comprises elements needed for expression of FIX without integration into a target genomic locus). In some methods, the F9 nucleic acid construct can be administered together with a nuclease agent described herein. The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target gene to create a cleavage site, the F9 nucleic acid construct can be inserted into the cleavage site to create a modified target gene, and FIX protein can be expressed from the modified target gene such that a therapeutically effective level of FIX expression is achieved in the subject. The FIX coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target sequence to create a cleavage site, the nucleic acid construct can be inserted into the cleavage site to create a modified ALB gene, and FIX protein can be expressed from the modified ALB gene such that a therapeutically effective level of FIX expression is achieved in the subject.

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of hemophilia B may comprise alleviating symptoms of hemophilia B. In one specific example, a method of preventing or inhibiting spontaneous bleeding in a subject having hemophilia B is provided. Hemophilia B is described in detail above and refers to a disorder caused by a missing or defective F9 gene or FIX polypeptide. The disorder includes conditions that are inherited and/or acquired (e.g., caused by a spontaneous mutation in the gene). The defective F9 gene or FIX polypeptide can result in reduced FIX level in the plasma and/or a reduced coagulation activity of FIX. Hemophilia B includes mild, moderate, and severe hemophilia B. For example, individuals with less than about 1% active factor are classified as having severe hemophilia, those with about 1-5% active factor have moderate hemophilia, and those with mild hemophilia have between about 5-40% of normal levels of active clotting factor. As used herein, “normal” or “healthy” individuals include those having between 50 and 160% of normal pooled plasma level of FIX activity and antigen levels. In one example, normal plasma FIX levels are about 3-5 μg/mL. In a specific example, normal FIX activity is considered to be about 100% of normal pooled plasma level of FIX activity or is considered to be 100% of normal pooled plasma level of FIX activity. In a specific example, normal plasma FIX levels are considered to be about 5 μg/mL or are considered to be 5 μg/mL. In some embodiments, the level of FIX (e.g., circulating FIX) can be measured by a coagulation and/or an immunologic assay. FIX procoagulant activity can be determined by the ability of the patient's plasma to correct the clotting time of FIX-deficient plasma.

In some methods, a therapeutically effective amount of the F9 nucleic acid construct or the composition comprising the F9 nucleic acid construct or the combination of the F9 nucleic acid construct and the plasma cell depleting agent or combination comprising the plasma cell depleting agent and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

In some methods, a therapeutically effective amount of the F9 nucleic acid construct or the composition comprising the F9 nucleic acid construct or the combination of the F9 nucleic acid construct and the B cell depleting agent and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding.

Therapeutic or pharmaceutical compositions comprising the compositions disclosed herein can be administered with suitable carriers, excipients, and other agents that are incorporated into formulations to provide improved transfer, delivery, tolerance, and the like. A multitude of appropriate formulations can be found in the formulary known to all pharmaceutical chemists: Remington's Pharmaceutical Sciences, Mack Publishing Company, Easton, PA. See also Powell et al. “Compendium of excipients for parenteral formulations” PDA (1998) J. Pharm. Sci. Technol. 52:238-311.

The compositions disclosed herein may be administered to relieve or prevent or decrease the severity of one or more of the symptoms of FIX deficiency or hemophilia B. Such symptoms are described in more detail elsewhere herein.

The methods disclosed herein can increase FIX protein levels and/or FIX activity levels in a cell or subject (e.g., circulating, serum, or plasma levels in a subject) and can comprise measuring FIX protein levels and/or activity levels in a cell or subject (e.g., circulating, serum, or plasma levels in a subject). In one example, the effectiveness of the treatment in a subject can be assessed by measuring serum or plasma FIX activity, wherein an increase in the subject's plasma level and/or activity of FIX indicates effectiveness of the treatment. In another example, effectiveness of the treatment can be determined by assessing clotting function in an aPTT assay and/or thrombin generation in an TGA-EA assay. In another example, effectiveness of the treatment can be determined by assessing the level or activity of Factor IX (e.g., circulating FIX) through a coagulation and/or an immunologic assay (e.g., a sandwich immunoassay, ELISA, or MSD).

In normal or healthy individuals, FIX activity and antigen levels vary between about 50% and 160% of normal pooled plasma, which is about 3-5 μg/mL, based on its purification from adult human plasma. See, e.g., Amiral et al. (1984) Clin. Chem. 30(9):1512-1516, herein incorporated by reference in its entirety for all purposes. In a specific example, normal FIX activity is considered to be about 100% of normal pooled plasma level of FIX activity or is considered to be 100% of normal pooled plasma level of FIX activity. In a specific example, normal plasma FIX levels are considered to be about 5 μg/mL or are considered to be 5 μg/mL. Individuals having less than 50% of normal plasma level of FIX activity and/or antigen levels are classified as having hemophilia. In particular, individuals with less than about 1% active FIX are classified as having severe hemophilia, while those with about 1-5% active FIX have moderate hemophilia. Individuals with mild hemophilia have between about 6-49% of normal levels of active clotting factor. In some embodiments, the level of circulating FIX can be measured by a coagulation and/or an immunologic assay using well known methods.

In some methods, plasma levels of FIX or FIX activity levels in a subject having hemophilia are increased to about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, or more, of normal level.

In some methods, circulating FIX protein levels are increased to about or at least about 0.05, about or at least about 0.1, about or at least about 0.2, about or at least about 0.5, about or at least about 1, about or at least about 2, about or at least about 3, or about or at least about 4 μg/mL. FIX protein levels may reach about 150 μg/mL, or more. In some methods, FIX protein levels are increased to at least about 4 μg/mL or about 4 μg/mL. In some methods, FIX protein levels are increased to about 4 μg/mL to about 5 μg/mL, about 4 μg/mL to 6 μg/mL, about 4 μg/mL to 8 μg/mL, about 4 μg/mL to about 10 μg/mL, or more. In some methods, FIX protein levels are increased to about 0.1 μg/mL to about 10 μg/mL, about 1 μg/mL to about 10 μg/mL, about 0.1 μg/mL to about 6 μg/mL, about 1 μg/mL to about 6 μg/mL, about 2 μg/mL to about 5 μg/mL, or about 3 μg/mL to about 5 μg/mL. For example, the compositions and methods disclosed herein are useful for increasing plasma levels of Factor IX in a subject having hemophilia to about 6, about 7, about 8, about 9, about 10, about 12, about 14, about 16, about 18, about 20, about 22, about 24, about 26, about 28, about 30, about 32, about 34, about 36, about 38, about 40, about 42, about 44, about 46, about 48, about 50, about 52, about 54, about 56, about 58, about 60, about 62, about 64, about 66, about 68, about 70, about 75, about 80, about 85, about 90, about 95, about 100, about 105, about 110, about 115, about 120, about 125, about 130, about 135, about 140, about 145, about 150 μg/mL, or more.

In some methods, plasma FIX activity and/or levels in a subject (e.g., having hemophilia) are increased by about or at least about 1%, about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 100%, about or at least about 110%, about or at least about 120%, about or at least about 130%, about or at least about 140%, about or at least about 150%, about or at least about 160%, about or at least about 170%, about or at least about 180%, about or at least about 190%, about or at least about 200%, or more, as compared to the subject's plasma level and/or activity of FIX before administration.

In some methods, FIX activity and/or protein levels in a cell or population of cells (e.g., liver cells, or hepatocytes) are increased by about or at least about 1%, about or at least about 2%, about or at least about 3%, about or at least about 4%, about or at least about 5%, about or at least about 6%, about or at least about 7%, about or at least about 8%, about or at least about 9%, about or at least about 10%, about or at least about 11%, about or at least about 12%, about or at least about 13%, about or at least about 14%, about or at least about 15%, about or at least about 16%, about or at least about 17%, about or at least about 18%, about or at least about 19%, about or at least about 20%, about or at least about 21%, about or at least about 22%, about or at least about 23%, about or at least about 24%, about or at least about 25%, about or at least about 26%, about or at least about 27%, about or at least about 28%, about or at least about 29%, about or at least about 30%, about or at least about 31%, about or at least about 32%, about or at least about 33%, about or at least about 34%, about or at least about 35%, about or at least about 36%, about or at least about 37%, about or at least about 38%, about or at least about 39%, about or at least about 40%, about or at least about 41%, about or at least about 42%, about or at least about 43%, about or at least about 44%, about or at least about 45%, about or at least about 46%, about or at least about 47%, about or at least about 48%, about or at least about 49%, about or at least about 50%, about or at least about 55%, about or at least about 60%, about or at least about 65%, about or at least about 70%, about or at least about 75%, about or at least about 80%, about or at least about 85%, about or at least about 90%, about or at least about 95%, about or at least about 100%, about or at least about 110%, about or at least about 120%, about or at least about 130%, about or at least about 140%, about or at least about 150%, about or at least about 160%, about or at least about 170%, about or at least about 180%, about or at least about 190%, about or at least about 200%, or more, as compared to the FIX activity and/or protein levels before administration (e.g., a normal level).

Some methods comprise expressing a therapeutically effective amount of FIX protein (e.g., achieving a therapeutically effective level of circulating FIX coagulation activity in an individual). Some methods comprise achieving FIX activity or expression levels of at least about 5% to about 50% of normal or at least about 50% to about 150% of normal. Some methods comprise achieving an increase in FIX activity or expression over the patient's baseline FIX activity or expression of at least about 1% to about 50% of normal FIX activity, or at least about 5% to about 50% of normal FIX activity, or at least about 50% to about 150% of normal FIX activity. Some methods comprise achieving FIX activity or expression levels of between about 40% and about 150% of normal (i.e., between 40% and 150% of normal). Some methods comprise achieving FIX activity or expression levels of between about 40% and about 100% of normal (e.g., between 40% and 100% of normal). Some methods comprise achieving FIX activity levels of between about 40% and about 150% of normal (i.e., between 40% and 150% of normal). Some methods comprise achieving FIX activity levels of between about 40% and about 100% of normal (e.g., between 40% and 100% of normal). Some methods comprise achieving FIX expression levels of between about 40% and about 150% of normal (i.e., between 40% and 150% of normal). Some methods comprise achieving FIX expression levels of between about 40% and about 100% of normal (e.g., between 40% and 100% of normal).

In a specific example, the FIX activity levels in a subject are increased to between about 1% to about 300%, about 1% to about 250%, about 1% to about 200%, about 1% to about 190%, about 1% to about 180%, about 1% to about 170%, about 1% to about 160%, about 1% to about 150%, about 1% to about 140%, about 1% to about 130%, about 1% to about 120%, about 1% to about 110%, about 1% to about 100%, about 1% to about 90%, about 1% to about 80%, about 1% to about 70%, about 1% to about 60%, about 1% to about 50%, about 5% to about 300%, about 5% to about 250%, about 5% to about 200%, about 5% to about 190%, about 5% to about 180%, about 5% to about 170%, about 5% to about 160%, about 5% to about 150%, about 5% to about 140%, about 5% to about 130%, about 5% to about 120%, about 5% to about 110%, about 5% to about 100%, about 5% to about 90%, about 5% to about 80%, about 5% to about 70%, about 5% to about 60%, about 5% to about 50%, about 15% to about 300%, about 15% to about 250%, about 15% to about 200%, about 15% to about 190%, about 15% to about 180%, about 15% to about 170%, about 15% to about 160%, about 15% to about 150%, about 15% to about 140%, about 15% to about 130%, about 15% to about 120%, about 15% to about 110%, about 15% to about 100%, about 15% to about 90%, about 15% to about 80%, about 15% to about 70%, about 15% to about 60%, about 15% to about 50%, about 10% to about 300%, about 15% to about 300%, about 20% to about 300%, about 25% to about 300%, about 30% to about 300%, about 35% to about 300%, about 40% to about 300%, about 45% to about 300%, about 50% to about 300%, about 55% to about 300%, about 60% to about 300%, about 65% to about 300%, about 70% to about 300%, about 75% to about 300%, about 80% to about 300%, about 85% to about 300%, about 90% to about 300%, about 95% to about 300%, about 100% to about 300%, about 10% to about 200%, about 15% to about 200%, about 20% to about 200%, about 25% to about 200%, about 30% to about 200%, about 35% to about 200%, about 40% to about 200%, about 45% to about 200%, about 50% to about 200%, about 55% to about 200%, about 60% to about 200%, about 65% to about 200%, about 70% to about 200%, about 75% to about 200%, about 80% to about 200%, about 85% to about 200%, about 90% to about 200%, about 95% to about 200%, about 100% to about 200%, about 10% to about 150%, about 15% to about 150%, about 20% to about 150%, about 25% to about 150%, about 30% to about 150%, about 35% to about 150%, about 40% to about 150%, about 45% to about 150%, about 50% to about 150%, about 55% to about 150%, about 60% to about 150%, about 65% to about 150%, about 70% to about 150%, about 75% to about 150%, about 80% to about 150%, about 85% to about 150%, about 90% to about 150%, about 95% to about 150%, about 100% to about 150%, about 50% to about 300%, about 50% to about 250%, about 50% to about 200%, about 50% to about 190%, about 50% to about 180%, about 50% to about 170%, about 50% to about 160%, about 50% to about 150%, about 50% to about 140%, about 50% to about 130%, about 50% to about 120%, about 50% to about 110%, about 50% to about 100% of normal FIX activity levels (e.g., between about 50% to about 150% of normal FIX activity levels).

In a specific example, the FIX activity levels in a subject are increased to between about 5% to about 200%, about 10% to about 190%, about 20% to about 180%, about 30% to about 170%, about 40% to about 160%, about 50% to about 150%, about 60% to about 140%, about 70% to about 130%, about 80% to about 120%, or about 90% to about 110% of normal FIX activity levels (e.g., to or about 100% of normal FIX activity levels).

In a specific example, the plasma FIX levels in a subject are increased to between about 1% to about 300%, about 1% to about 250%, about 1% to about 200%, about 1% to about 190%, about 1% to about 180%, about 1% to about 170%, about 1% to about 160%, about 1% to about 150%, about 1% to about 140%, about 1% to about 130%, about 1% to about 120%, about 1% to about 110%, about 1% to about 100%, about 1% to about 90%, about 1% to about 80%, about 1% to about 70%, about 1% to about 60%, about 1% to about 50%, about 5% to about 300%, about 5% to about 250%, about 5% to about 200%, about 5% to about 190%, about 5% to about 180%, about 5% to about 170%, about 5% to about 160%, about 5% to about 150%, about 5% to about 140%, about 5% to about 130%, about 5% to about 120%, about 5% to about 110%, about 5% to about 100%, about 5% to about 90%, about 5% to about 80%, about 5% to about 70%, about 5% to about 60%, about 5% to about 50%, about 15% to about 300%, about 15% to about 250%, about 15% to about 200%, about 15% to about 190%, about 15% to about 180%, about 15% to about 170%, about 15% to about 160%, about 15% to about 150%, about 15% to about 140%, about 15% to about 130%, about 15% to about 120%, about 15% to about 110%, about 15% to about 100%, about 15% to about 90%, about 15% to about 80%, about 15% to about 70%, about 15% to about 60%, about 15% to about 50%, about 10% to about 300%, about 15% to about 300%, about 20% to about 300%, about 25% to about 300%, about 30% to about 300%, about 35% to about 300%, about 40% to about 300%, about 45% to about 300%, about 50% to about 300%, about 55% to about 300%, about 60% to about 300%, about 65% to about 300%, about 70% to about 300%, about 75% to about 300%, about 80% to about 300%, about 85% to about 300%, about 90% to about 300%, about 95% to about 300%, about 100% to about 300%, about 10% to about 200%, about 15% to about 200%, about 20% to about 200%, about 25% to about 200%, about 30% to about 200%, about 35% to about 200%, about 40% to about 200%, about 45% to about 200%, about 50% to about 200%, about 55% to about 200%, about 60% to about 200%, about 65% to about 200%, about 70% to about 200%, about 75% to about 200%, about 80% to about 200%, about 85% to about 200%, about 90% to about 200%, about 95% to about 200%, about 100% to about 200%, about 10% to about 150%, about 15% to about 150%, about 20% to about 150%, about 25% to about 150%, about 30% to about 150%, about 35% to about 150%, about 40% to about 150%, about 45% to about 150%, about 50% to about 150%, about 55% to about 150%, about 60% to about 150%, about 65% to about 150%, about 70% to about 150%, about 75% to about 150%, about 80% to about 150%, about 85% to about 150%, about 90% to about 150%, about 95% to about 150%, about 100% to about 150%, about 50% to about 300%, about 50% to about 250%, about 50% to about 200%, about 50% to about 190%, about 50% to about 180%, about 50% to about 170%, about 50% to about 160%, about 50% to about 150%, about 50% to about 140%, about 50% to about 130%, about 50% to about 120%, about 50% to about 110%, about 50% to about 100% of normal plasma FIX levels (e.g., between about 50% to about 150% of normal plasma FIX levels).

In a specific example, the plasma FIX levels in a subject are increased to between about 5% to about 200%, about 10% to about 190%, about 20% to about 180%, about 30% to about 170%, about 40% to about 160%, about 50% to about 150%, about 60% to about 140%, about 70% to about 130%, about 80% to about 120%, or about 90% to about 110% of normal plasma FIX levels (e.g., to or about 100% of normal plasma FIX levels).

In a specific example, the plasma FIX levels in a subject are increased to between about 0.25 μg/mL to about 15 μg/mL, about 0.25 μg/mL to about 14 μg/mL, about 0.25 μg/mL to about 13 μg/mL, about 0.25 μg/mL to about 12 μg/mL, about 0.25 μg/mL to about 11 μg/mL, about 0.25 μg/mL to about 10 μg/mL, about 0.25 μg/mL to about 9 μg/mL, about 0.25 μg/mL to about 8 μg/mL, about 0.25 μg/mL to about 7 μg/mL, about 0.25 μg/mL to about 6 μg/mL, about 0.25 μg/mL to about 5 μg/mL, about 0.25 μg/mL to about 4 μg/mL, about 0.25 μg/mL to about 3 μg/mL, about 0.25 μg/mL to about 2 μg/mL, about 0.25 μg/mL to about 1 μg/mL, about 0.75 μg/mL to about 15 μg/mL, about 0.75 μg/mL to about 14 μg/mL, about 0.75 μg/mL to about 13 μg/mL, about 0.75 μg/mL to about 12 μg/mL, about 0.75 μg/mL to about 11 μg/mL, about 0.75 μg/mL to about 10 μg/mL, about 0.75 μg/mL to about 9 μg/mL, about 0.75 μg/mL to about 8 μg/mL, about 0.75 μg/mL to about 7 μg/mL, about 0.75 μg/mL to about 6 μg/mL, about 0.75 μg/mL to about 5 μg/mL, about 0.75 μg/mL to about 4 μg/mL, about 0.75 μg/mL to about 3 μg/mL, about 0.75 μg/mL to about 2 μg/mL, about 0.75 μg/mL to about 1 μg/mL, about 0.5 μg/mL to about 15 μg/mL, about 0.75 μg/mL to about 15 μg/mL, about 1 μg/mL to about 15 μg/mL, about 2 μg/mL to about 15 μg/mL, about 3 μg/mL to about 15 μg/mL, about 4 μg/mL to about 15 μg/mL, about 5 μg/mL to about 15 μg/mL, about 0.5 μg/mL to about 10 μg/mL, about 0.75 μg/mL to about 10 μg/mL, about 1 μg/mL to about 10 μg/mL, about 2 μg/mL to about 10 μg/mL, about 3 μg/mL to about 10 μg/mL, about 4 μg/mL to about 10 μg/mL, about 5 μg/mL to about 10 μg/mL, about 0.5 μg/mL to about 7.5 μg/mL, about 0.75 μg/mL to about 7.5 μg/mL, about 1 μg/mL to about 7.5 μg/mL, about 2 μg/mL to about 7.5 μg/mL, about 2.5 μg/mL to about 7.5 μg/mL, about 3 μg/mL to about 7.5 μg/mL, about 4 μg/mL to about 7.5 μg/mL, about 5 μg/mL to about 7.5 μg/mL, about 2.5 μg/mL to about 15 μg/mL, about 2.5 μg/mL to about 14 μg/mL, about 2.5 μg/mL to about 13 μg/mL, about 2.5 μg/mL to about 12 μg/mL, about 2.5 μg/mL to about 11 μg/mL, about 2.5 μg/mL to about 10 μg/mL, about 2.5 μg/mL to about 9 μg/mL, about 2.5 μg/mL to about 8 μg/mL, about 2.5 μg/mL to about 7 μg/mL, about 2.5 μg/mL to about 6 μg/mL, about 2.5 μg/mL to about 5 μg/mL, about 3 μg/mL to about 15 μg/mL, about 3 μg/mL to about 14 μg/mL, about 3 μg/mL to about 13 μg/mL, about 3 μg/mL to about 12 μg/mL, about 3 μg/mL to about 11 μg/mL, about 3 μg/mL to about 10 μg/mL, about 3 μg/mL to about 9 μg/mL, about 3 μg/mL to about 8 μg/mL, about 3 μg/mL to about 7 μg/mL, about 3 μg/mL to about 6 μg/mL, about 3 μg/mL to about 5 μg/mL (e.g., between about 2.5 μg/mL to about 7.5 μg/mL or between about 3 μg/mL to about 5 μg/mL). In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In a specific example, the plasma FIX levels in a subject are increased to between about 0.5 μg/mL to about 10 μg/mL, about 1 μg/mL to about 9 μg/mL, about 2 μg/mL to about 8 μg/mL, about 3 μg/mL to about 7 μg/mL, or about 4 μg/mL to about 6 μg/mL (e.g., to about 5 μg/mL). In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In a specific example, the FIX activity levels in a subject are increased to at least about 15% of normal FIX activity levels. In a specific example, the plasma FIX levels in a subject are increased to at least about 15% of normal plasma FIX levels. In a specific example, the plasma FIX levels in a subject are increased to at least about 0.75 μg/mL. For example, the method can be a method of preventing or inhibiting spontaneous bleeding in a subject having hemophilia B, and the FIX activity levels in a subject are increased to at least about 15% of normal FIX activity levels or the plasma FIX levels in a subject are increased to at least about 15% of normal plasma FIX levels or the plasma FIX levels in a subject are increased to at least about 0.75 μg/mL.

In a specific example, the FIX activity levels in a subject are increased to no more than about 300%, no more than about 250%, no more than about 200%, or no more than about 150% of normal FIX activity levels. In a specific example, the plasma FIX levels in a subject are increased to no more than about 300%, no more than about 250%, no more than about 200%, or no more than about 150% of normal plasma FIX levels.

In a specific example, a subject has severe hemophilia, and the FIX activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has severe hemophilia, and the plasma FIX levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has severe hemophilia, and the FIX activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has severe hemophilia, and the plasma FIX levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

In a specific example, a subject has moderate hemophilia, and the FIX activity levels in the subject are increased to at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has moderate hemophilia, and the plasma FIX levels in the subject are increased to at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has moderate hemophilia, and the FIX activity levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has moderate hemophilia, and the plasma FIX levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

In a specific example, a subject has mild hemophilia, and the FIX activity levels in the subject are increased to at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal FIX activity levels.

In a specific example, a subject has mild hemophilia, and the plasma FIX levels in the subject are increased to at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal plasma FIX levels.

In a specific example, a subject has mild hemophilia, and the FIX activity levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal FIX activity levels.

In a specific example, a subject has mild hemophilia, and the plasma FIX levels in the subject are increased to more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal plasma FIX levels.

Some methods comprise achieving a durable effect, such as an at least 1 month, at least 2 months, at least 6 months, at least 1 year, or at least 2 year effect. Some methods comprise achieving the therapeutic effect in a durable and sustained manner, such as an at least 1 month, at least 2 months, at least 6 months, at least 1 year, or at least 2 year effect. In some methods, the increased circulating FIX activity and/or expression level is stable for at least 1 month, at least 2 months, at least 6 months, at least 1 year, or more. In some methods, a steady-state activity and/or level of FIX protein is achieved by at least 7 days, at least 14 days, or at least 28 days. In additional methods, the method comprises maintaining FIX activity and/or levels after a single dose for at least 1, at least 2, at least 4, or at least 6 months, or at least 1, at least 2, at least 3, at least 4, or at least 5 years. Some methods comprise achieving a durable or sustained effect in a human, such as an at least at least 8 weeks, at least 24 weeks, for example, at least 1 year (52 weeks), or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased FIX activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of FIX in a human is achieved by at least 7 days, at least 14 days, or at least 28 days, optionally at least 56 days, at least 80 days, or at least 96 days. In additional methods, the method comprises maintaining FIX activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the FIX can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the FIX can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the FIX is maintained at a level higher than the expression or activity of the FIX prior to treatment (i.e., the subject's baseline). In some methods, expression or activity of the FIX is considered sustained if it is maintained at a therapeutically effective level of expression or activity. Relative durations, in other organisms, are understood based, e.g., on life span and developmental stages, are covered within the disclosure above. In some methods, expression or activity of the FIX is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the FIX, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the FIX is sustained when the neonatal subject becomes an adult. In some methods, the expression of the FIX is sustained for the lifetime of the subject or neonatal subject.

In some methods, the expression or activity of the FIX is at least 50% of the expression or activity of the FIX at a peak level of expression measured for the human subject at 24 weeks after the administering. In some methods, the expression or activity of the FIX is at least 50% of the expression or activity of the FIX at a peak level of expression measured for the human subject at one year after the administering. In some methods, the expression or activity of the FIX is at least 60% of the expression or activity of the FIX at a peak level of expression measured for the human subject at 24 weeks after the administering. In some methods, expression or activity of the FIX is at least 50% of the expression or activity of the FIX at a peak level of expression measured for the human subject at two years after the administering. In some methods, the expression or activity of the FIX is at least 60% of the expression or activity of the FIX at a peak level of expression measured for the human subject at 2 years after the administering. In some methods, the expression or activity of the FIX is at least 60% of the expression or activity of the FIX at a peak level of expression measured for the human subject at 24 weeks after the administering.

In some methods, combination therapies are used comprising the any of the compositions for expressing FIX disclosed herein together with an additional therapy suitable for treating hemophilia B or a FIX deficiency. As one example, the methods of described herein can be combined with the use of other hemostatic agents, blood factors, and medications. For example, the subject may be administered a therapeutically effective amount of one or more factors selected from the group consisting of factor XI, factor XII, prekallikrein, high molecular weight kininogen (HMWK), factor V, factor VII, factor VIII, factor X, factor XIII, factor II, factor VIIa, and von Willebrands factor. Additionally, or alternatively, treatment may further comprise administering a procoagulant, such as an activator of the intrinsic coagulation pathway, including factor Xa, factor IXa, factor XIa, factor XIIa, and VIIla, prekallekrein, and high-molecular weight kininogen; or an activator of the extrinsic coagulation pathway, including tissue factor, factor VIIa, factor Va, and factor Xa.

### B. Pompe Disease

In some methods disclosed herein, the polypeptide of interest is a multidomain therapeutic protein disclosed herein (e.g., a lysosomal alpha-glucosidase linked to a CD63-binding delivery domain or TfR-binding delivery domain), and the enzyme deficiency is GAA deficiency or Pompe disease. See, e.g., PCT/US2023/061858 and U.S. Ser. No. 18/163,698, each of which is herein incorporated by reference in its entirety for all purposes. In such methods, the nucleic acid constructs and compositions disclosed herein can be used in methods of introducing a nucleic acid construct encoding a polypeptide of interest in a cell or population of cells in a subject, methods of inserting or integrating a nucleic acid construct encoding a polypeptide of interest into a target genomic locus in a cell or population of cells in a subject, methods of expressing a polypeptide of interest (e.g., from a target genomic locus) in a cell or population of cells in a subject, methods of reducing glycogen accumulation in a cell or a population of cells or a tissue in a subject, methods of treating Pompe disease or GAA deficiency in a subject, and methods or preventing or reducing the onset of a sign or symptom of Pompe disease or GAA deficiency in a subject.

The multidomain therapeutic protein compositions disclosed herein (e.g., multidomain therapeutic protein nucleic acid constructs, or multidomain therapeutic protein nucleic acid constructs in combination with plasma cell depleting agents or combinations comprising plasma cell depleting agents and nuclease agents (e.g., CRISPR/Cas systems)) are useful for the treatment of GAA deficiency or Pompe disease and/or ameliorating at least one symptom associated with GAA deficiency or Pompe disease (e.g., as compared to a control, untreated subject). The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are also useful for preventing or reducing the onset of a sign or symptom of GAA deficiency or Pompe disease (e.g., as compared to a control, untreated subject). Likewise, the compositions disclosed herein can be used for the preparation of a pharmaceutical composition or medicament for treating a subject having GAA deficiency or Pompe disease.

The multidomain therapeutic protein compositions disclosed herein (e.g., multidomain therapeutic protein nucleic acid constructs, or multidomain therapeutic protein nucleic acid constructs in combination with B cell depleting agents (e.g., anti-CD20×CD3 antigen-binding molecule) and nuclease agents (e.g., CRISPR/Cas systems)) are useful for the treatment of GAA deficiency or Pompe disease and/or ameliorating at least one symptom associated with GAA deficiency or Pompe disease (e.g., as compared to a control, untreated subject). The multidomain therapeutic protein compositions disclosed herein (e.g., multidomain therapeutic protein nucleic acid constructs, or multidomain therapeutic protein nucleic acid constructs in combination with the nuclease agents (e.g., CRISPR/Cas systems)) are also useful for preventing or reducing the onset of a sign or symptom of GAA deficiency or Pompe disease (e.g., as compared to a control, untreated subject). Likewise, the compositions disclosed herein can be used for the preparation of a pharmaceutical composition or medicament for treating a subject having GAA deficiency or Pompe disease.

With respect to GAA deficiency or Pompe disease, the terms “treat,” “treated,” “treating,” and “treatment,” include the administration of the multidomain therapeutic domain nucleic acid constructs disclosed herein (e.g., together with a plasma cell depleting agent or combination comprising the plasma cell depleting agent and a nuclease agent disclosed herein, or together with a B cell depleting agent (e.g., in a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV)) to subjects to prevent or delay the onset of the symptoms, complications, or biochemical indicia of GAA deficiency or Pompe disease, alleviating the symptoms or arresting or inhibiting further development of GAA deficiency or Pompe disease. Treatment may be prophylactic (to prevent or delay the onset of GAA deficiency or Pompe disease, or to prevent the manifestation of clinical or subclinical symptoms thereof) or therapeutic suppression or alleviation of symptoms after the manifestation of GAA deficiency or Pompe disease.

GAA deficiency refers expression and/or activity levels of GAA being lower in the subject (e.g., neonatal subject) than normal GAA expression and/or activity levels, such that the normal functions of GAA are not fully carried out in the subject (e.g., resulting in Pompe disease). Pompe disease is also known as acid maltase deficiency, acid maltase deficiency disease, alpha-1,4-glucosidase deficiency, AMD, deficiency of alpha-glucosidase, GAA deficiency, glycogen storage disease type II, glycogenosis type IL, GSD IL, GSD2, and Pompe's disease.

Pompe disease is an inherited disorder caused by the buildup of glycogen in the body's cells. The accumulation of glycogen in certain organs and tissues, especially muscles, impairs their ability to function normally. Different types of Pompe disease differ in severity and the age at which they appear. These types are known as infantile-onset Pompe disease (classic infantile-onset, and non-classic infantile-onset) and late-onset Pompe disease. Subjects with late-onset Pompe disease have higher GAA enzyme levels than are found in infantile-onset forms of the disease, but generally less than 40 percent of normal enzyme activity. Classic infantile-onset Pompe disease patients typically have less than 1 percent of GAA enzyme activity, while those with non-classic forms usually have less than 10 percent.

The classic form of infantile-onset Pompe disease begins within a few months of birth. Some phenotypes, such as cardiomyopathies, can be present at birth. Infants with this disorder typically experience muscle weakness (myopathy), poor muscle tone (hypotonia), an enlarged liver (hepatomegaly), and heart defects. Affected infants may also fail to gain weight and grow at the expected rate (failure to thrive) and have breathing problems. If untreated, this form of Pompe disease leads to death from heart failure in the first year of life.

The non-classic form of infantile-onset Pompe disease usually appears by age 1. It is characterized by delayed motor skills (such as rolling over and sitting) and progressive muscle weakness. The heart may be abnormally large (cardiomegaly), but affected individuals usually do not experience heart failure. The muscle weakness in this disorder leads to serious breathing problems, and most children with non-classic infantile-onset Pompe disease live only into early childhood.

The late-onset type of Pompe disease may not become apparent until later in childhood, adolescence, or adulthood. Late-onset Pompe disease is usually milder than the infantile-onset forms of this disorder and is less likely to involve the heart. Most individuals with late-onset Pompe disease experience progressive muscle weakness, especially in the legs and the trunk, including the muscles that control breathing. As the disorder progresses, breathing problems can lead to respiratory failure.

Mutations in the GAA gene can cause Pompe disease. The GAA gene encodes an enzyme called alpha-glucosidase. This enzyme is active in lysosomes. The enzyme normally breaks down glycogen into glucose. Mutations in the GAA gene prevent acid alpha-glucosidase from breaking down glycogen effectively, which allows this sugar to build up to toxic levels in lysosomes. This buildup damages organs and tissues throughout the body, particularly the muscles, leading to the progressive signs and symptoms of Pompe disease.

Since this is a genetic condition, the people who get this disease inherit it from a parent. It is common, however, that neither parent shows any symptoms. The disease is rare. In the United States, only 1 person in 40,000 is affected by Pompe disease. It can affect both males and females of all ethnic groups.

Symptoms of Pompe disease can be different, depending on when the disease makes itself present. For classic type, infant symptoms can include the following: weak muscles, poor muscle tone, enlarged liver, failure to gain weight and grow at the expected rate (failure to thrive), trouble breathing, feeding problems, infections in the respiratory system, and problems with hearing. For non-classic type, infant symptoms can include the following: motor skills delayed (such as rolling over and sitting), muscles get steadily weaker, abnormally large heart, and breathing problems. For late-onset type, symptoms can include the following: the legs and the trunk get steadily weaker, breathing problems, enlarged heart, increasing difficulty in walking, muscle pain over a large area, loss of the ability to exercise, falling often, frequent lung infections, shortness of breath when the person pushes himself or herself, headaches in the morning, becoming tired during the day, losing weight, cannot swallow as easily as before, irregular heartbeat, increased difficulty hearing, and higher levels of creatine kinase.

Pathology in Pompe disease can begin long before subjects present with symptoms. Pompe disease can be diagnosed by taking a blood sample, and enzymes in the blood are studied and counted. Confirmation can be made via DNA testing. For example, GAA enzyme activity can be measured by flow-injection tandem mass spectrometry, and full sequencing of the GAA gene is performed in newborns with low GAA enzyme activity. See, e.g., Ficicioglu et al. (2020) Int. J. Neonatal Screen. 6(4):89, Tang et al. (2020) Int. J. Neonatal Screen. 6(1):9, and Klug et al. (2020) Int. J. Neonatal Screen 6(1):11, each of which is herein incorporated by reference in its entirety for all purposes. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry. These can be combined with genetic analyses to diagnose in infantile and late onset Pompe disease. Asymptomatic subjects can be considered to have Pompe disease if diagnosed by genetic screening. For example, a subject described herein is considered to have Pompe disease, even if they are asymptomatic, if they have reduced GAA activity and a pathogenic GAA variant or mutation. Pathogenic GAA mutations and variants associated with Pompe disease are known. See, e.g., Ficicioglu et al. (2020) Int. J. Neonatal Screen. 6(4):89, Tang et al. (2020) Int. J. Neonatal Screen. 6(1):9, and Klug et al. (2020) Int. J. Neonatal Screen 6(1):11, each of which is herein incorporated by reference in its entirety for all purposes.

As is the case for several other lysosomal diseases, Pompe disease is currently treated by enzyme replacement therapy (ERT). Recombinant human GAA is delivered by intravenous infusion into patients every other week. While ERT has been successful in treating the cardiac manifestations of Pompe disease, skeletal muscle and the central nervous system (CNS) remain minimally treated by ERT.

The methods described herein can be used to treat a lysosomal alpha-glucosidase (GAA) deficiency in a subject in need thereof (e.g., a subject with Pompe disease). The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the expressed multidomain therapeutic protein is delivered to and internalized by skeletal muscle, liver, or heart tissue in the subject. In some methods, the expressed multidomain therapeutic protein is delivered to and internalized by skeletal muscle or heart tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by skeletal muscle, liver, and heart tissue in the subject. In some methods, the expressed multidomain therapeutic protein is delivered to and internalized by skeletal muscle and heart tissue in the subject. In some methods, the expressed multidomain therapeutic protein is delivered to and internalized by skeletal muscle, liver, heart, or central nervous system tissue in the subject. In some methods, the expressed multidomain therapeutic protein is delivered to and internalized by skeletal muscle, heart, or central nervous system tissue in the subject. In some methods, the expressed multidomain therapeutic protein is delivered to and internalized by skeletal muscle tissue in the subject. In some methods, the expressed multidomain therapeutic protein is delivered to and internalized by liver tissue in the subject. In some methods, the expressed multidomain therapeutic protein is delivered to and internalized by heart tissue in the subject. In some methods, the expressed multidomain therapeutic protein is delivered to and internalized by central nervous system tissue in the subject. In some methods, the expressed multidomain therapeutic protein is delivered to and internalized by skeletal muscle, liver, heart, and central nervous system tissue in the subject. In some methods, the expressed multidomain therapeutic protein is delivered to and internalized by skeletal muscle, heart, and central nervous system tissue in the subject. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age.

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a plasma cell depleting agent or combination comprising the plasma cell depleting agent such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with a B cell depleting agent (e.g., anti-CD20×CD3 antigen-binding molecule) such that a therapeutically effective level of multidomain therapeutic protein or GAA expression or a therapeutically effective level of circulating multidomain therapeutic protein or GAA is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of reducing glycogen accumulation in a cell or a population of cells or a tissue in a subject in need thereof (e.g., a subject with Pompe disease). Similarly, provided are methods of reducing glycogen accumulation in a cell or a population of cells. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age.

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a plasma cell depleting agent or combination comprising the plasma cell depleting agent such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with a B cell depleting agent (e.g., anti-CD20×CD3 antigen-binding molecule) such that a therapeutically effective level of multidomain therapeutic protein or GAA expression or a therapeutically effective level of circulating multidomain therapeutic protein or GAA is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Also provided are methods of treating Pompe disease in a subject. The Pompe disease can be any type of Pompe disease (e.g., infantile-onset Pompe disease (classic infantile-onset or non-classic infantile-onset) or late-onset Pompe disease). For example, the subject can have infantile-onset Pompe disease (e.g., classical infantile-onset Pompe disease). Pompe disease is described in more detail elsewhere herein. In some methods, the method reduces glycogen accumulation in skeletal muscle, liver, or heart tissue in the subject. In some methods, the method reduces glycogen accumulation in skeletal muscle or heart tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. For example, glycogen accumulation can be reduced in skeletal muscle, liver, and heart tissue in the subject. For example, glycogen accumulation can be reduced in skeletal muscle and heart tissue in the subject. In some methods, the method reduces glycogen accumulation in skeletal muscle, liver, heart, or central nervous system tissue in the subject. In some methods, the method reduces glycogen accumulation in skeletal muscle, heart, or central nervous system tissue in the subject. In some methods, the method reduces glycogen accumulation in skeletal muscle tissue in the subject. In some methods, the method reduces glycogen accumulation in liver tissue in the subject. In some methods, the method reduces glycogen accumulation in heart tissue in the subject. In some methods, the method reduces glycogen accumulation in central nervous system tissue in the subject. For example, glycogen accumulation can be reduced in skeletal muscle, liver, heart, and central nervous system tissue in the subject. For example, glycogen accumulation can be reduced in skeletal muscle, heart, and central nervous system tissue in the subject. In some cases, glycogen levels are reduced to wild type levels. In some cases, glycogen levels in skeletal muscle, heart, and/or central nervous system tissue are reduced to levels comparable to wild type levels at the same age. In some methods, the method improves muscle strength in the subject (e.g., restores muscle strength to wild type levels). In some methods, the method prevents loss of muscle strength in the subject compared to a control. In some methods, the method results in the subject having muscle strength comparable to wild type levels at the same age.

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a plasma cell depleting agent or combination comprising the plasma cell depleting agent such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with a B cell depleting agent (e.g., anti-CD20×CD3 antigen-binding molecule) such that a therapeutically effective level of multidomain therapeutic protein or GAA expression or a therapeutically effective level of circulating multidomain therapeutic protein or GAA is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Treatment refers to any administration or application of a therapeutic for disease or disorder in a subject, and includes inhibiting the disease, arresting its development, relieving one or more symptoms of the disease, curing the disease, or preventing reoccurrence of one or more symptoms of the disease. For example, treatment of Pompe disease may comprise alleviating symptoms of Pompe disease. Pompe disease is described in detail above and refers to a disorder caused by a missing or defective GAA gene or GAA polypeptide. The defective GAA gene or GAA polypeptide can result in reduced GAA expression and/or an activity of GAA.

Also provided are methods of preventing or reducing the onset of a sign or symptom of Pompe disease in a subject (e.g., as compared to an untreated, control subject). By preventing is meant the sign or symptom of the Pompe disease never becomes present. Such signs and symptoms are well-known and are described in more detail elsewhere herein. The Pompe disease can be any type of Pompe disease (e.g., infantile-onset Pompe disease (classic infantile-onset or non-classic infantile-onset) or late-onset Pompe disease). For example, Pompe disease can be infantile-onset Pompe disease (e.g., classical infantile-onset Pompe disease). Pompe disease is described in more detail elsewhere herein. In some methods, the method prevents or reduces glycogen accumulation in skeletal muscle, liver, or heart tissue in the subject. In some methods, the method prevents or reduces glycogen accumulation in skeletal muscle or heart tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. For example, glycogen accumulation can be prevented or reduced in skeletal muscle, liver, and heart tissue in the subject. For example, glycogen accumulation can be prevented or reduced in skeletal muscle and heart tissue in the subject. In some methods, the method prevents or reduces glycogen accumulation in skeletal muscle, liver, heart, or central nervous system tissue in the subject. In some methods, the method prevents or reduces glycogen accumulation in skeletal muscle, heart, or central nervous system tissue in the subject. In some methods, the method prevents or reduces glycogen accumulation in skeletal muscle tissue in the subject. In some methods, the method prevents or reduces glycogen accumulation in liver tissue in the subject. In some methods, the method prevents or reduces glycogen accumulation in heart tissue in the subject. In some methods, the method prevents or reduces glycogen accumulation in central nervous system tissue in the subject. For example, glycogen accumulation can be prevented or reduced in skeletal muscle, liver, heart, and central nervous system tissue in the subject. For example, the onset of glycogen accumulation can be prevented or reduced in skeletal muscle, heart, and central nervous system tissue in the subject.

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject in combination with a plasma cell depleting agent or combination comprising the plasma cell depleting agent such that a therapeutically effective level of multidomain therapeutic protein or GAA expression or a therapeutically effective level of circulating multidomain therapeutic protein or GAA is achieved in the subject. The plasma cell depleting agent or combination comprising the plasma cell depleting agent can be administered prior to, simultaneously with, or after the nucleic acid construct. In one example, the plasma cell depleting agent or combination comprising the plasma cell depleting agent is administered prior to the nucleic acid construct. In another example, the plasma cell depleting agent or combination comprising the plasma cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The plasma cell depleting agent or combination comprising the plasma cell depleting agent can be administered prior to, simultaneously with, or after the nuclease agent. In one example, the plasma cell depleting agent or combination comprising the plasma cell depleting agent is administered prior to the nuclease agent. In another example, the plasma cell depleting agent or combination comprising the plasma cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

Such methods can comprise administering any of the multidomain therapeutic protein nucleic acid constructs described herein (or any of the compositions comprising a multidomain therapeutic protein nucleic acid construct described herein, including, for example, vectors or lipid nanoparticles) to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with a B cell depleting agent (e.g., anti-CD20×CD3 antigen-binding molecule) such that a therapeutically effective level of multidomain therapeutic protein or GAA expression or a therapeutically effective level of circulating multidomain therapeutic protein or GAA is achieved in the subject. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) simultaneously with the B cell depleting agent. In some embodiments, a plasma cell depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, an immunoglobulin depleting agent is not administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. In some embodiments, neither a plasma cell depleting agent nor an immunoglobulin depleting agent is administered to the subject (e.g., a subject without preexisting immunity to the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent, such as, e.g., AAV) in combination with the B cell depleting agent. The B cell depleting agent can be administered prior to, simultaneously with, or after the nucleic acid construct. In one example, the B cell depleting agent is administered prior to the nucleic acid construct. In another example, the B cell depleting agent is administered prior to and after the nucleic acid construct. In some methods, the multidomain therapeutic protein nucleic acid construct or composition comprising the multidomain therapeutic protein nucleic acid construct can be administered without a nuclease agent (e.g., if the multidomain therapeutic protein nucleic acid construct comprises elements needed for expression of multidomain therapeutic protein without integration into a target genomic locus). In some methods, the multidomain therapeutic protein nucleic acid construct can be administered together with a nuclease agent described herein (e.g., simultaneously or sequentially in any order). The B cell depleting agent can be administered prior to, simultaneously with, or after the nuclease agent. In one example, the B cell depleting agent is administered prior to the nuclease agent. In another example, the B cell depleting agent is administered prior to and after the nuclease agent. The nuclease agent can cleave a nuclease target sequence within a target genomic locus (e.g., target gene), the multidomain therapeutic protein nucleic acid construct can be inserted into the target genomic locus to create a modified target genomic locus, and the multidomain therapeutic protein can be expressed from the modified target genomic locus (e.g., such that a therapeutically effective level of multidomain therapeutic protein or GAA expression or a therapeutically effective level of circulating multidomain therapeutic protein or GAA is achieved in the subject). The multidomain therapeutic protein coding sequence can be operably linked to an endogenous promoter at the target genomic locus upon integration into the target genomic locus, or it can be operably linked to an exogenous promoter present in the nucleic acid construct. In one example, the nuclease agent is a CRISPR/Cas system, and the target gene is ALB (e.g., intron 1 of ALB). In such methods, the guide RNA can bind to the Cas protein and target the Cas protein to the guide RNA target sequence in intron 1 of the ALB gene, the Cas protein can cleave the guide RNA target, the nucleic acid construct can be inserted into the ALB gene to create a modified ALB gene, and multidomain therapeutic protein can be expressed from the modified ALB gene (e.g., such that a therapeutically effective level of multidomain therapeutic protein or GAA expression or a therapeutically effective level of circulating multidomain therapeutic protein or GAA is achieved in the subject).

In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the plasma cell depleting agent or combination comprising the plasma cell depleting agent and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. In a specific example, serum levels of at least about 2 μg/mL or at least about 5 μg/mL of the multidomain therapeutic protein are considered therapeutically effective and correspond to complete correction of glycogen storage in muscles.

In some methods, a therapeutically effective amount of the multidomain therapeutic protein nucleic acid construct or the composition comprising the multidomain therapeutic protein nucleic acid construct or the combination of the multidomain therapeutic protein nucleic acid construct and the B cell depleting agent and the nuclease agent (e.g., CRISPR/Cas system) is administered to the subject. A therapeutically effective amount is an amount that produces the desired effect for which it is administered. The exact amount will depend on the purpose of the treatment, and will be ascertainable by one skilled in the art using known techniques. See, e.g., Lloyd (1999) The Art, Science and Technology of Pharmaceutical Compounding. In a specific example, serum levels of at least about 2 μg/mL or at least about 5 μg/mL of the multidomain therapeutic protein are considered therapeutically effective and correspond to complete correction of glycogen storage in muscles.

The methods disclosed herein can increase multidomain therapeutic protein or GAA protein levels and/or multidomain therapeutic protein or GAA activity levels in a cell or subject (e.g., circulating, serum, or plasma levels in a subject) and can comprise measuring multidomain therapeutic protein or GAA protein levels and/or multidomain therapeutic protein or GAA activity levels in a cell or subject (e.g., circulating, serum, or plasma levels in a subject). In one example, the methods result in increased expression of the multidomain therapeutic protein in the subject compared to a method comprising administering an episomal expression vector encoding the multidomain therapeutic protein. For example, the methods can result in increased serum levels of the multidomain therapeutic protein in the subject compared to a method comprising administering an episomal expression vector encoding the multidomain therapeutic protein. The methods can also result in increased multidomain therapeutic protein activity or GAA activity in the subject compared to a method comprising administering an episomal expression vector encoding the multidomain therapeutic protein. Levels of circulating multidomain therapeutic protein or GAA activity can be measured by using well-known methods.

In some methods, GAA activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, GAA activity and/or expression levels in a subject are increased to about or at least about 40%, about or at least about 50%, about or at least about 75%, or at least about 100%, or more, of normal level. In certain embodiments, the level of expression or activity is measured in a cell or tissue in which a sign or symptom of the GAA loss of function is present. For example, when the loss of function results in muscle dysfunction, the level or activity of the multidomain therapeutic protein or GAA is measured in a muscle cell. It is understood that depending on the exogenous protein, the level of activity of the multidomain therapeutic protein may not compare 1:1 with a native GAA protein based on weight. In such embodiment, the relative activity of the multidomain therapeutic protein and the native GAA can be compared. In certain embodiments, the loss of function is nearly complete such that a relative activity cannot be determined. In certain embodiments, the comparison is made to an appropriate control subject. Selection of an appropriate control subject is within the ability of those of skill in the art. In certain embodiments, the level of expression is sufficient to treat at least one sign or symptom resulting from the loss of function of the GAA. GAA activity can be assessed by any known method. For example, to assess GAA activity (or deficiencies of activity), blood-based assays can measure GAA activity in dried blood spots or fresh blood. GAA activity can also be measured in fibroblasts from a skin biopsy or muscle biopsy. Other secondary measures can be measuring urine glucose tetrasaccharides by mass spectrometry.

In some methods, circulating multidomain therapeutic protein levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, multidomain therapeutic protein levels are at least about 1 μg/mL or about 1 μg/mL. In some methods, multidomain therapeutic protein levels are at least about 2 μg/mL or about 2 μg/mL. In some methods, multidomain therapeutic protein levels are at least about 5 μg/mL or about 5 μg/mL. In some methods, multidomain therapeutic protein levels are about 1 μg/mL to about 30 μg/mL, about 2 μg/mL to about 30 μg/mL, about 3 μg/mL to about 30 μg/mL, about 4 μg/mL to about 30 μg/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in multidomain therapeutic protein levels of about 2 μg/mL to about 30 μg/mL or 2 μg/mL to about 20 μg/mL. For example, the method can result in multidomain therapeutic protein levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of GAA or the multidomain therapeutic protein over the subject's baseline expression and/or activity (i.e., expression and/or activity prior to administration). In some methods, the method increases expression and/or activity of GAA over the subject's baseline expression and/or activity (i.e., expression and/or activity prior to administration. In some methods, GAA activity and/or GAA expression or serum levels in a subject are increased by about or at least about 10%, about or at least about 25%, about or at least about 50%, about or at least about 75%, or about or at least about 100%, or more, as compared to the subject's GAA expression or serum levels and/or activity (e.g., GAA activity) before administration (i.e., the subject's baseline levels). It is understood that depending on the multidomain therapeutic protein, the level of activity of the multidomain therapeutic protein may not compare 1:1 with a native protein based on weight. In such embodiment, the relative activity of the multidomain therapeutic protein and the native GAA can be compared. In certain embodiments, the loss of function is nearly complete such that a relative activity cannot be determined. In certain embodiments, the level of expression is sufficient to treat at least one sign or symptom resulting from the loss of function of the GAA.

In some methods, the method increases expression and/or activity of the multidomain therapeutic protein over the cell's baseline expression and/or activity (i.e., expression and/or activity prior to administration). In some methods, the method increases expression and/or activity of GAA over the cell's baseline expression and/or activity (i.e., expression and/or activity prior to administration. In some methods, GAA activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the GAA activity and/or expression levels before administration (i.e., the subject's baseline levels). It is understood that depending on the multidomain therapeutic protein, the level of activity of the multidomain therapeutic protein may not compare 1:1 with a native GAA protein based on weight. In such embodiment, the relative activity of the multidomain therapeutic protein and the native GAA protein can be compared. In certain embodiments, the GAA loss of function is nearly complete such that a relative activity cannot be determined. In certain embodiments, the level of expression is sufficient to treat at least one sign or symptom resulting from the loss of function of the GAA.

In a specific example, the GAA activity levels in a subject are increased to no more than about 300%, no more than about 250%, no more than about 200%, or no more than about 150% of normal GAA activity levels.

In a specific example, the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic infantile-onset Pompe disease), and the GAA activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels. In a specific example, a subject has infantile-onset Pompe disease (e.g., classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has infantile-onset Pompe disease (e.g., classic or non-classic infantile-onset Pompe disease), and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels). In a specific example, a subject has infantile-onset Pompe disease (e.g., classic or non-classic infantile-onset Pompe disease), and the GAA activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels.

In a specific example, a subject has late-onset Pompe disease, and the GAA activity levels in the subject are increased to at least about 2% at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels (e.g., at least about 40%, at least about 45%, at least about 50%, at least about 55%, at least about 60%, at least about 65%, at least about 70%, at least about 75%, at least about 80%, at least about 85%, at least about 90%, or at least about 100% of normal GAA activity levels).

In a specific example, a subject has infantile-onset Pompe disease (e.g., classic infantile-onset Pompe disease), and the GAA activity levels in the subject are increased to more than about 1%, more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels. In a specific example, a subject has infantile-onset Pompe disease (e.g., classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels

In a specific example, a subject has infantile-onset Pompe disease (e.g., classic or non-classic infantile-onset Pompe disease), and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels). In a specific example, a subject has infantile-onset Pompe disease (e.g., classic or non-classic infantile-onset Pompe disease), and the GAA activity levels in the subject are increased to more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels.

In a specific example, a subject has late-onset Pompe disease, and the GAA activity levels in the subject are increased to more than about 2% more than about 5%, more than about 10%, more than about 15%, more than about 20%, more than about 25%, more than about 30%, more than about 35%, more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels (e.g., more than about 40%, more than about 45%, more than about 50%, more than about 55%, more than about 60%, more than about 65%, more than about 70%, more than about 75%, more than about 80%, more than about 85%, more than about 90%, or more than about 100% of normal GAA activity levels).

In some methods, the method results in increased expression of the multidomain therapeutic protein in the subject (e.g., neonatal subject) compared to a method comprising administering an episomal expression vector encoding the polypeptide of interest in a control subject. In some methods, the method results in increased serum levels of the multidomain therapeutic protein in the subject (e.g., neonatal subject) compared to a method comprising administering an episomal expression vector encoding the polypeptide of interest to a control subject.

In some methods, the method increases expression or activity of the multidomain therapeutic protein or GAA over the subject's (e.g., neonatal subject's) baseline expression or activity of the multidomain therapeutic protein or GAA (i.e., any percent change in expression that is larger than typical error bars). In some methods, the method results in expression of the multidomain therapeutic protein or GAA at a detectable level above zero, e.g., at a statistically significant level, a clinically relevant level.

Some methods comprise achieving a durable or sustained effect in a human, such as an at least at least 8 weeks, at least 24 weeks, for example, at least 1 year, or optionally at least 2 year effect, and in some embodiments, at least 3 year, at least 4 year, or at least 5 year effect. Some methods comprise achieving the therapeutic effect in a human in a durable and sustained manner, such as an at least 8 weeks, at least 24 weeks, for example, at least 1 year, or optionally at least 2 year effect, and in some embodiments, at least 3 year, at least 4 year, or at least 5 year effect. In some methods, the increased multidomain therapeutic protein or GAA activity and/or expression level in a human is stable for at least at least 8 weeks, at least 24 weeks, for example, at least 1 year, optionally at least 2 years, and in some embodiments, at least 3 years, at least 4 years, or at least 5 years. In some methods, a steady-state activity and/or level of multidomain therapeutic protein or GAA in a human is achieved by at least 7 days, at least 14 days, or at least 28 days, optionally at least 56 days, at least 80 days, or at least 96 days. In additional methods, the method comprises maintaining multidomain therapeutic protein or GAA activity and/or levels after a single dose in a human for at least 8 weeks, at least 16 weeks, or at least 24 week, or in some embodiments at least 1 year, or at least 2 years, optionally at least 3 years, at least 4 years, or at least 5 years. For example, expression of the multidomain therapeutic protein or GAA can be sustained in the human subject for at least about 8 weeks, at least about 12 weeks, at least about 24 weeks, in certain embodiments, at least about 1 year, or at least about 2 years after treatment, and in some embodiments, at least 3 years, at least 4 years, or at least 5 years after treatment. Likewise, activity of the multidomain therapeutic protein or GAA can be sustained in the human subject for at least about 8 weeks, at least about 12 weeks, at least about 24 weeks, in certain embodiments for at least about 1 year, or at least about 2 years after treatment, and in some embodiments, at least 3 years, at least 4 years, or at least 5 years after treatment. In some methods, expression or activity of the multidomain therapeutic protein or GAA is maintained at a level higher than the expression or activity of the multidomain therapeutic protein or GAA prior to treatment (i.e., the subject's baseline). In some methods, expression or activity of the multidomain therapeutic protein or GAA is considered sustained if it is maintained at a therapeutically effective level of expression or activity. Relative durations, in other organisms, are understood based, e.g., on life span and developmental stages, are covered within the disclosure above. In some methods, expression or activity of the multidomain therapeutic protein or GAA is considered “sustained” if the expression or activity in a human at six months after administration, one year after administration, or two years after administration, the expression or activity is at least 50% of the expression or activity of the peak level of expression or activity measured for that subject. In certain embodiments, at six months, e.g., at 24 weeks to 28 weeks, after administration the expression or activity is at least 50%, 55%, 60%, 65%, 70%, 75% or 80% of the expression or activity of the peak level of expression or activity measured for that subject. In certain embodiments, at one year, i.e., about 12 months, e.g., at 11-13 months, after administration the expression or activity is at least 50%, 55%, 60%, 65%, 70%, 75% or 80% of the expression or activity of the peak level of expression or activity measured for that subject. In certain embodiments, at two years, i.e., about 24 months, e.g., at 23-25 months, after administration the expression or activity is at least 50%, 55%, 60%, 65%, 70%, 75% or 80% of the expression or activity of the peak level of expression or activity measured for that subject. In certain embodiments, at six months after administration the expression or activity is at least 50%, preferably at least 60% of the expression or activity of the peak level of expression or activity measured for that subject. In certain embodiments, at one year after administration the expression or activity is at least 50%, preferably at least 60% of the expression or activity of the peak level of expression or activity measured for that subject. In certain embodiments, at two years after administration the expression or activity is at least 50%, preferably at least 60% of the expression or activity of the peak level of expression or activity measured for that subject. In preferred embodiments, the subject has routine monitoring of expression or activity levels of the polypeptide, e.g., weekly, monthly, particularly early after administration, e.g., within the first six months. Periodic measurements may establish that the effect on expression or activity is sustained at, e.g., 6 months after administration, one year after administration, or two years after administration. In some methods in neonatal subjects, the expression of the multidomain therapeutic protein or GAA is sustained when the neonatal subject becomes an adult. In some methods, the expression of the multidomain therapeutic protein or GAA is sustained for the lifetime of the subject or neonatal subject.

In some methods, the expression or activity of the multidomain therapeutic protein is at least 50% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at one year after the administering. In some methods, the expression or activity of the multidomain therapeutic protein is at least 60% of the expression or activity of the multidomain therapeutic protein at a peak level of expression measured for the subject at 24 weeks after the administering. In some methods, the expression or activity of the multidomain therapeutic protein is at least 50% of the expression or activity of the multidomain therapeutic protein at a peak level of expression measured for the subject at two years after the administering. In some methods, the expression or activity of the multidomain therapeutic protein is at least 60% of the expression or activity of the multidomain therapeutic protein at a peak level of expression measured for the subject at 2 years after the administering. In some methods, the expression or activity of the multidomain therapeutic protein is at least 60% of the expression or activity of the multidomain therapeutic protein at a peak level of expression measured for the subject at 24 weeks after the administering.

### C. Dosage and Administration Regimens

In some embodiments, an amount of a plasma cell depleting agent, e.g., an antigen-binding molecule that binds to B cell maturation antigen (BCMA) and CD3 (e.g., an anti-BCMA×CD3 bispecific antibody), a B cell depleting agent (e.g., anti-CD19 and anti-CD20 antibodies, or a CD20×CD3 antigen-binding molecule (e.g., REGN1979)), an immunoglobulin depleting agent such as a neonatal Fc receptor (FcRn) blocker (e.g., efgartigimod alfa), and/or an immunogen (e.g., nucleic acid construct, nuclease agent or CRISPR/Cas system, e.g., in an immunogenic delivery vehicle) (e.g., an immunogenic delivery vehicle), or pharmaceutical composition thereof which is administered to a subject according to the methods disclosed herein is a therapeutically effective amount. As used herein, the phrase “therapeutically effective amount” means an amount that produces the desired effect for which it is administered. The subject can be from any suitable species, such as eukaryotic or mammalian subjects (e.g., non-human mammalian subject or human subject). A mammal can be, for example, a non-human mammal, a human, a rodent, a rat, a mouse, or a hamster. Other non-human mammals include, for example, non-human primates, e.g., monkeys and apes. The term “non-human” excludes humans. Specific examples include, but are not limited to, humans, rodents, mice, rats, and non-human primates. In a specific example, the subject is a human. The human may be a patient. Likewise, cells can be any suitable type of cell. In a specific example, the cell or cells are a liver cell or liver cells such as a hepatocyte or hepatocytes (e.g., human liver cell(s) or human hepatocyte(s)). In some embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)), the immunoglobulin depleting agent (e.g., efgartigimod alfa), and/or the immunogen (e.g., nucleic acid construct, nuclease agent or CRISPR/Cas system, e.g., in an immunogenic delivery vehicle) (e.g., an immunogenic delivery vehicle), or pharmaceutical composition thereof, is administered to a subject as a weight-based dose. A “weight-based dose” (e.g., a dose in mg/kg) is a dose of the plasma cell depleting agent, the B cell depleting agent, the immunoglobulin depleting agent, and/or the immunogenic delivery vehicle that will change depending on the subject's weight.

In other embodiments, the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)), the immunoglobulin depleting agent (e.g., efgartigimod alfa), and/or the immunogen (e.g., nucleic acid construct, nuclease agent or CRISPR/Cas system, e.g., in an immunogenic delivery vehicle) (e.g., an immunogenic delivery vehicle), is administered as a fixed dose. A “fixed dose” (e.g., a dose in mg) means that one dose of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle) (e.g., an immunogenic delivery vehicle), is used for all subjects regardless of any specific subject-related factors, such as weight. In one particular embodiment, a fixed dose of a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle) is based on a predetermined weight or age.

Typically, a suitable dose of the plasma cell depleting agent and/or the B cell depleting agent and/or the immunoglobulin depleting agent and/or the immunogen (e.g., nucleic acid construct, nuclease agent or CRISPR/Cas system, e.g., in an immunogenic delivery vehicle) can be in the range of about 0.001 to about 200.0 milligram per kilogram body weight of the recipient, generally in the range of about 1 to 50 mg per kilogram body weight. For example, the plasma cell depleting agent and/or the B cell depleting agent and/or the immunoglobulin depleting agent and/or the immunogen (e.g., nucleic acid construct, nuclease agent or CRISPR/Cas system, e.g., in an immunogenic delivery vehicle) can be administered at about 0.1 mg/kg, about 0.2 mg/kg, about 0.5 mg/kg, about 1 mg/kg, about 1.5 mg/kg, about 2 mg/kg, about 3 mg/kg, about 5 mg/kg, about 10 mg/kg, about 15 mg/kg, about 20 mg/kg, about 25 mg/kg, about 30 mg/kg, about 40 mg/kg, or about 50 mg/kg per single dose. Values and ranges intermediate to the recited values are also intended to be part of this disclosure.

In some embodiments, the plasma cell depleting agent (e.g., an anti-BCMA×CD3 bispecific antibody) is administered at a dose of about 25, about 20 to about 30, about 15 to about 35, about 10 to about 40, about 10 to about 25, about 15 to about 25, about 20 to about 25, about 25 to about 30, about 25 to about 35, or about 25 to about 40 mg/kg. In some embodiments, the plasma cell depleting agent (e.g., an anti-BCMA×CD3 bispecific antibody) is administered at a dose of about 20 to about 30 mg/kg. In some embodiments, the plasma cell depleting agent (e.g., an anti-BCMA×CD3 bispecific antibody) is administered at a dose of about 25 mg/kg.

In some embodiments, the immunoglobulin depleting agent (e.g., FcRn blocker, such as efgartigimod alfa) is administered at a dose of about 20, about 15 to about 25, about 10 to about 30, about 5 to about 35, about 5 to about 20, about 10 to about 20, about 15 to about 20, about 20 to about 25, about 20 to about 30, or about 20 to about 35 mg/kg. In some embodiments, the immunoglobulin depleting agent (e.g., FcRn blocker, such as efgartigimod alfa) is administered at a dose of about 10 to about 20 mg/kg. In some embodiments, the immunoglobulin depleting agent (e.g., FcRn blocker, such as efgartigimod alfa) is administered at a dose of about 25 to about 24 mg/kg. In some embodiments, the immunoglobulin depleting agent (e.g., FcRn blocker, such as efgartigimod alfa) is administered at a dose of about 20 mg/kg. In some embodiments, the immunoglobulin depleting agent (e.g., FcRn blocker, such as efgartigimod alfa) is administered at a dose of about 10 mg/kg.

In some embodiments, the immunoglobulin depleting agent (e.g., FcRn blocker, such as efgartigimod alfa) is administered at a dose of about 10 mg/kg weekly for about 1 week, about 2 weeks, about 3 weeks, about 4 weeks, about 5 weeks, about 6 weeks, about 7 weeks, about 8 weeks, about 9 weeks, or about 10 weeks, or more. In some embodiments, the immunoglobulin depleting agent (e.g., FcRn blocker, such as efgartigimod alfa) is administered at a dose of about 10 mg/kg weekly for about 4 weeks. In various embodiments, such as when a dose of an immunoglobulin depleting agent (e.g., FcRn blocker, such as efgartigimod alfa) is administered, e.g., in combination with a plasma cell depleting agent and/or a B cell depleting agent, and optionally, an immunogen (e.g., an immunogenic delivery vehicle such as, e.g., AAV), described herein, the first dose of the immunoglobulin depleting agent may be delayed as compared to the first dose of the plasma cell depleting agent, the first dose of the B cell depleting agent, and/or the first dose of the immunogen. In some embodiments, the first dose of the immunoglobulin depleting agent is delayed by about 5 to about 20 days as compared to first dose of the plasma cell depleting agent, the first dose of the B cell depleting agent, and/or the first dose of the immunogen. In some embodiments, the first dose of the immunoglobulin depleting agent is delayed by about 7 to about 15 days as compared to first dose of the plasma cell depleting agent, the first dose of the B cell depleting agent, and/or the first dose of the immunogen. In some embodiments, the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen. In some embodiments, the first dose of the immunoglobulin depleting agent is delayed by about 12 days as compared to first dose of the plasma cell depleting agent, the first dose of the B cell depleting agent, and/or the first dose of the immunogen. In some embodiments, the first dose of the immunoglobulin depleting agent is delayed by about 13 days as compared to first dose of the plasma cell depleting agent, the first dose of the B cell depleting agent, and/or the first dose of the immunogen. In some embodiments, the first dose of the immunoglobulin depleting agent is delayed by about 14 days as compared to first dose of the plasma cell depleting agent, the first dose of the B cell depleting agent, and/or the first dose of the immunogen.

In one specific embodiment, the immunoglobulin depleting agent (e.g., FcRn blocker, such as efgartigimod alfa) is administered at a dose of about 10 mg/kg weekly for about 4 weeks and the first dose of the immunoglobulin depleting agent is delayed by about 9 to about 11 days as compared to first dose of the plasma cell depleting agent, the first dose of the B cell depleting agent, and/or the first dose of the immunogen. In some embodiments, the first dose of the immunoglobulin depleting agent is delayed by about 9 days as compared to first dose of the plasma cell depleting agent, the first dose of the B cell depleting agent, and/or the first dose of the immunogen. In some embodiments, the first dose of the immunoglobulin depleting agent is delayed by about 10 days as compared to first dose of the plasma cell depleting agent, the first dose of the B cell depleting agent, and/or the first dose of the immunogen. In some embodiments, the first dose of the immunoglobulin depleting agent is delayed by about 11 days as compared to first dose of the plasma cell depleting agent, the first dose of the B cell depleting agent, and/or the first dose of the immunogen.

In some embodiments, the B cell depleting agent (e.g., anti-CD19 antibody or anti-CD20 antibody) is administered at a dose of about 20, about 15 to about 25, about 10 to about 30, about 5 to about 35, about 5 to about 20, about 10 to about 20, about 15 to about 20, about 20 to about 25, about 20 to about 30, or about 20 to about 35 mg/kg. In some embodiments, the B cell depleting agent (e.g., anti-CD19 antibody or anti-CD20 antibody) is administered at a dose of about 10 to about 20 mg/kg. In some embodiments, the B cell depleting agent (e.g., anti-CD19 antibody or anti-CD20 antibody) is administered at a dose of about 25 to about 24 mg/kg. In some embodiments, the B cell depleting agent (e.g., anti-CD19 antibody or anti-CD20 antibody) is administered at a dose of about 20 mg/kg.

In some embodiments, the B cell depleting agent (e.g., anti-CD20×CD3 bispecific antibody) is administered at a dose of about 0.4 to about 0.6, 0.3 to about 0.7, 0.2, to about 0.8, 0.1 to about 0.9, 0.1 to about 0.5, 0.2 to about 0.5, 0.3 to about 0.5, 0.4 to about 0.5, 0.5 to about 0.6, 0.5 to about 0.7, 0.5 to about 0.8, or 0.5 to about 0.9 mg/kg. In some embodiments, the B cell depleting agent (e.g., anti-CD20×CD3 bispecific antibody or anti-CD19 antibody or anti-CD20 antibody) is administered at a dose of about 0.4 to about 0.6 mg/kg. In some embodiments, the B cell depleting agent (e.g., anti-CD20×CD3 bispecific antibody) is administered at a dose of about 0.3 to about 0.7 mg/kg. In some embodiments, the B cell depleting agent (e.g., anti-CD20×CD3 bispecific antibody) is administered at a dose of about 0.5 mg/kg.

In some embodiments, the plasma cell depleting agent and/or the B cell depleting agent and/or the immunoglobulin depleting agent and/or the immunogen (e.g., nucleic acid construct, nuclease agent or CRISPR/Cas system, e.g., in an immunogenic delivery vehicle) is administered as a fixed dose of between about 5 mg to about 2500 mg. In some embodiments, the plasma cell depleting agent and/or the B cell depleting agent and/or the immunoglobulin depleting agent is administered as a fixed dose of about 5 mg, about 10 mg, about 15 mg, about 20 mg, about 25 mg, about 30 mg, about 50 mg, about 75 mg, about 100 mg, about 125 mg, about 150 mg, about 175 mg, 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1500 mg, about 2000 mg, or about 2500 mg. Values and ranges intermediate to the recited values are also intended to be part of this disclosure.

In one embodiment, for a plasma cell depleting agent (e.g., an anti-BCMA/anti-CD3 bispecific antibody), a therapeutically effective amount can be from about 0.05 mg to about 500 mg, from about 1 mg to about 500 mg, from about 10 mg to about 450 mg, from about 50 mg to about 400 mg, from about 75 mg to about 350 mg, or from about 100 mg to about 300 mg of the antibody. For example, in various embodiments, the amount of the plasma cell depleting agent is about 0.05 mg, about 0.1 mg, about 1.0 mg, about 1.5 mg, about 2.0 mg, about 5 mg, about 10 mg, about 15 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, or about 500 mg, of the plasma cell depleting agent.

In some embodiments, the plasma cell depleting agent (e.g., an anti-BCMA×CD3 bispecific antibody) and/or the B cell depleting agent (e.g., anti-CD19/CD20 antibodies, or a CD20×CD3 antigen-binding molecule (e.g., REGN1979)) and/or the immunoglobulin depleting agent (e.g., efgartigimod alfa) and/or the immunogen (e.g., nucleic acid construct, nuclease agent or CRISPR/Cas system, e.g., in an immunogenic delivery vehicle) (e.g., an immunogenic delivery vehicle) is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the immunogenic delivery vehicle can be administered at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In some embodiments, the immunogen (e.g., nucleic acid construct, nuclease agent or CRISPR/Cas system, e.g., in an immunogenic delivery vehicle) (e.g., an immunogenic delivery vehicle) can be administered at a dosing frequency of about four times a year, twice a year, once a week, once every two years, once every three years, once every four years, once every five years, once every six years, once every eight years, once every twelve years, or less frequently so long as a therapeutic response is achieved.

Dose ranges and frequency of administration of an immunogen (e.g., nucleic acid construct, nuclease agent or CRISPR/Cas system, e.g., in an immunogenic delivery vehicle), e.g., an immunogenic delivery vehicle such as a vector (e.g., a viral vector such as an AAV vector) described herein can vary depending on the nature of, and/or the medical condition, as well as parameters of a specific subject and the route of administration used. As a non-limiting example, vector compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the vector compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×10⁸ pfu to about 1×10¹⁰ pfu, while an adult human subject can receive a dose from about 1×10¹⁰ pfu to about 1×10¹² pfu.

In some embodiments, multiple doses of a plasma cell depleting agent (e.g., an anti-BCMA×CD3 bispecific antibody), a B cell depleting agent (e.g., anti-CD19/CD20 antibodies, or a CD20×CD3 antigen-binding molecule (e.g., REGN1979)), an immunoglobulin depleting agent (e.g., efgartigimod alfa), and/or an immunogen (e.g., nucleic acid construct, nuclease agent or CRISPR/Cas system, e.g., in an immunogenic delivery vehicle) (e.g., an immunogenic delivery vehicle) are administered to a subject over a defined time course. In some embodiments, the methods of the present disclosure comprise sequentially administering to a subject multiple doses of the plasma cell depleting agent (e.g., an anti-BCMA×CD3 bispecific antibody), the B cell depleting agent (e.g., anti-CD19/CD20 antibodies, or a CD20×CD3 antigen-binding molecule (e.g., REGN1979)), the immunoglobulin depleting agent (e.g., efgartigimod alfa), and/or the immunogen (e.g., nucleic acid construct, nuclease agent or CRISPR/Cas system, e.g., in an immunogenic delivery vehicle) (e.g., an immunogenic delivery vehicle).

In some embodiments, the immunogen (e.g., nucleic acid construct, nuclease agent or CRISPR/Cas system, e.g., in an immunogenic delivery vehicle) (e.g., an immunogenic delivery vehicle such as a vector, e.g., an AAV vector) may be administered in accordance with a repeat dosing regimen wherein the immunogen (e.g., nucleic acid construct, nuclease agent or CRISPR/Cas system, e.g., in an immunogenic delivery vehicle) (e.g., an immunogenic delivery vehicle) may be administered a first time (e.g., in an initial dose) and then re-administered any number of subsequent times thereafter at any amount over the time course of treatment of a subject. For example, the immunogen (e.g., nucleic acid construct, nuclease agent or CRISPR/Cas system, e.g., in an immunogenic delivery vehicle) may be re-administered once, twice, three times, four times, five times, six times, seven times, eight times, nine times, ten times, or more, over the time course of the treatment of a subject which can occur over any number of days, weeks, or years. In some embodiments, when the immunogen (e.g., nucleic acid construct, nuclease agent or CRISPR/Cas system, e.g., in an immunogenic delivery vehicle) (e.g., an immunogenic delivery vehicle) comprises a vector, e.g., a viral vector such as an AAV vector, the vector which is administered first in a repeat dosing regimen may comprise the same vector which is re-administered second in the repeat dosing regimen, or any number of subsequent times thereafter. In some embodiments, when the immunogen (e.g., nucleic acid construct, nuclease agent or CRISPR/Cas system, e.g., in an immunogenic delivery vehicle) (e.g., an immunogenic delivery vehicle) comprises a vector, e.g., a viral vector such as an AAV vector, the vector which is administered first in a repeat dosing regimen may comprise a different vector than is re-administered second in the repeat dosing regimen, or any number of subsequent times thereafter.

In some embodiments, immunogen (e.g., nucleic acid construct, nuclease agent or CRISPR/Cas system, e.g., in an immunogenic delivery vehicle) (e.g., an immunogenic delivery vehicle), e.g., a viral vector such as an AAV vector, may be administered in accordance with a stepwise dosing regimen. Stepwise dosing of an immunogen (e.g., nucleic acid construct, nuclease agent or CRISPR/Cas system, e.g., in an immunogenic delivery vehicle) can refer to breaking up (i.e., dividing) dosing of the same immunogen (e.g., nucleic acid construct, nuclease agent or CRISPR/Cas system, e.g., in an immunogenic delivery vehicle) over multiple administrations. In some embodiments, the dosing of the same immunogen (e.g., nucleic acid construct, nuclease agent or CRISPR/Cas system, e.g., in an immunogenic delivery vehicle) is broken up once, twice, three times, four times, five times, six times, seven times, eight times, nine times, ten times, or more, over the time course of the treatment of a subject which can occur over any number of days, weeks, or years. In some embodiments, when a stepwise dose regimen is used in the administration of an immunogen (e.g., nucleic acid construct, nuclease agent or CRISPR/Cas system, e.g., in an immunogenic delivery vehicle), e.g., an immunogenic delivery vehicle, e.g., a viral vector such as an AAV vector, the stepwise dosing regimen may result in a gradual increase in therapeutic transgene levels with each administration of the immunogen (e.g., nucleic acid construct, nuclease agent or CRISPR/Cas system, e.g., in an immunogenic delivery vehicle). Without wishing to be bound by theory, a stepwise dosing regimen used in the administration of an immunogen (e.g., nucleic acid construct, nuclease agent or CRISPR/Cas system, e.g., in an immunogenic delivery vehicle) comprising an immunogenic delivery vehicle, e.g., a viral vector such as an AAV vector, can result in enhanced control over transgene expression in a cell and/or subject, since for some transgenes too much expression can result in its own pathology.

In some embodiments, an immunogen (e.g., nucleic acid construct, nuclease agent or CRISPR/Cas system, e.g., in an immunogenic delivery vehicle) (e.g., an immunogenic delivery vehicle) is administered to a subject in combination with a plasma cell depleting agent, a B cell depleting agent, and/or an immunoglobulin depleting agent. The plasma cell depleting agent and/or the B cell depleting agent and/or the immunoglobulin depleting agent can be administered prior to, simultaneously with, or after the immunogen (e.g., nucleic acid construct, nuclease agent or CRISPR/Cas system, e.g., in an immunogenic delivery vehicle). In one example, the plasma cell depleting agent and/or B cell depleting agent and/or immunoglobulin depleting agent is administered prior to the immunogen (e.g., nucleic acid construct, nuclease agent or CRISPR/Cas system, e.g., in an immunogenic delivery vehicle). In another example, the plasma cell depleting agent and/or B cell depleting agent and/or immunoglobulin depleting agent is administered prior to and after the immunogen (e.g., nucleic acid construct, nuclease agent or CRISPR/Cas system, e.g., in an immunogenic delivery vehicle). In another example, the plasma cell depleting agent and/or B cell depleting agent and/or immunoglobulin depleting agent is administered simultaneously with the immunogen (e.g., nucleic acid construct, nuclease agent or CRISPR/Cas system, e.g., in an immunogenic delivery vehicle). In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen, e.g., 2-4 days afterwards as plasma cell formation may be limited during the initial lag period. In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen, e.g., 2-4 days afterwards as B cell formation may be limited during the initial lag period. Administration of the B cell depleting agent shortly after the administration of the immunogen may prevent B cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In some embodiments, when the plasma cell depleting agent and/or the B cell depleting agent and/or the immunoglobulin depleting agent is administered prior to, simultaneously with, and/or after the immunogen (e.g., nucleic acid construct, nuclease agent or CRISPR/Cas system, e.g., in an immunogenic delivery vehicle) (e.g., an immunogenic delivery vehicle), the plasma cell depleting agent and/or the B cell depleting agent and/or the immunoglobulin depleting agent is administered once, twice, three times, four times, five times, six times, seven times, eight times, nine times, ten times, eleven times, twelve times, thirteen times, fourteen times, fifteen times, sixteen times, seventeen times, eighteen times, nineteen times, or twenty times or more, prior to, simultaneously with, and/or after the administration of the immunogen (e.g., nucleic acid construct, nuclease agent or CRISPR/Cas system, e.g., in an immunogenic delivery vehicle) (e.g., an immunogenic delivery vehicle). In some embodiments, when an immunogen (e.g., nucleic acid construct, nuclease agent or CRISPR/Cas system, e.g., in an immunogenic delivery vehicle) is administered in accordance with a repeat dosing regimen, a plasma cell depleting agent and/or a B cell depleting agent and/or an immunoglobulin depleting agent may be administered any number of times prior to, simultaneously with, and/or after a first and/or second administration of the immunogen (e.g., nucleic acid construct, nuclease agent or CRISPR/Cas system, e.g., in an immunogenic delivery vehicle), and/or any number of subsequent administrations of the immunogen thereafter. Without wishing to be bound by theory, when a plasma cell depleting agent and/or a B cell depleting agent and/or an immunoglobulin depleting agent is administered to inhibit an immune response to an immunogen (e.g., nucleic acid construct, nuclease agent or CRISPR/Cas system, e.g., in an immunogenic delivery vehicle) in a subject in need thereof, e.g., an anti-drug antibody response to an immunogenic protein, the plasma cell depleting agent and/or the B cell depleting agent and/or the immunoglobulin depleting agent may be co-administered (e.g., administered prior to, simultaneously with, and/or after the immunogen) to prevent the response of the immune system of the subject on each dose of the immunogen (e.g., nucleic acid construct, nuclease agent or CRISPR/Cas system, e.g., in an immunogenic delivery vehicle). As an example, an immunogen comprising a bacterial IgG cleaving enzyme IdeS/imlifidase may be administered to a subject for overcoming AAV pre-existing immunity; however, IdeS itself is immunogenic and can only be administered once. Co-administration of a plasma cell depleting agent and/or a B cell depleting agent and/or an immunoglobulin depleting agent described herein with IdeS/imlifidase can prevent the de novo response to IdeS protein.

According to certain embodiments of the present disclosure, a plasma cell depleting agent and/or a B cell depleting agent and/or an immunoglobulin depleting agent may be administered to a subject separately from an immunogen (e.g., nucleic acid construct, nuclease agent or CRISPR/Cas system, e.g., in an immunogenic delivery vehicle) (e.g., an immunogenic delivery vehicle) described herein.

In some embodiments, when a plasma cell depleting agent and/or a B cell depleting agent and/or an immunoglobulin depleting agent and an immunogen (e.g., nucleic acid construct, nuclease agent or CRISPR/Cas system, e.g., in an immunogenic delivery vehicle) (e.g., an immunogenic delivery vehicle) are administered separately, the plasma cell depleting agent and/or the B cell depleting agent and/or the immunoglobulin depleting agent may be administered simultaneously with the administration of the immunogen (e.g., nucleic acid construct, nuclease agent or CRISPR/Cas system, e.g., in an immunogenic delivery vehicle). In some embodiments, the plasma cell depleting agent and/or the B cell depleting agent and/or the immunoglobulin depleting agent is administered one or more times during the administration of the immunogen (e.g., nucleic acid construct, nuclease agent or CRISPR/Cas system, e.g., in an immunogenic delivery vehicle). In some embodiments, the immunogen (e.g., nucleic acid construct, nuclease agent or CRISPR/Cas system, e.g., in an immunogenic delivery vehicle) is administered one or more times during the administration of the plasma cell depleting agent and/or the B cell depleting agent and/or the immunoglobulin depleting agent. In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen, e.g., 2-4 days afterwards as plasma cell formation may be limited during the initial lag period. In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In some embodiments, the immunoglobulin depleting agent is administered after an initial dose of the plasma cell depleting agent. In some embodiments, the immunoglobulin depleting agent is administered after an initial dose of the plasma cell depleting agent and after an initial dose of the B cell depleting agent.

In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen, e.g., 2-4 days afterwards as B cell formation may be limited during the initial lag period. Administration of the B cell depleting agent shortly after the administration of the immunogen may prevent B cell formation and persistence elicited by administration of the immunogen to immunologically naïve patients. In some embodiments, the B cell depleting agent is administered prior to and after administration of the immunogen (e.g., to deplete existing B cells and then to maintain the depletion thereafter).

In some embodiments, a B cell depleting agent is administered alone (e.g., without a plasma cell depleting agent). In some embodiments, a B cell depleting agent is administered prior to a plasma cell depleting agent. In some embodiments, a B cell depleting agent is administered simultaneously with a plasma cell depleting agent. In some embodiments, a B cell depleting agent is administered subsequent to a plasma cell depleting agent. In some embodiments, a B cell depleting agent is administered prior to and subsequent to a plasma cell depleting agent. In some embodiments, a B cell depleting agent is administered prior to and simultaneously with a plasma cell depleting agent. In some embodiments, a B cell depleting agent is administered simultaneously with and subsequent to a plasma cell depleting agent. In theory, B cell depletion could be conducted before or after plasma cell depletion with the same effect, provided that B cells remain depleted up until the time of dosing with the immunogen.

In some embodiments, an immunoglobulin depleting agent is administered subsequent to a plasma cell depleting agent. In some embodiments, an immunoglobulin depleting agent is administered subsequent to a B cell depleting agent. In some embodiments, an immunoglobulin depleting agent is administered subsequent to a plasma cell depleting agent and a B cell depleting agent. For example, if the plasma cell depleting agent is an anti-BCMA×CD3 bispecific antibody, administering the immunoglobulin depleting agent after the plasma cell depleting agent will prevent more rapid clearance of the plasma cell depleting agent. For example, if the B cell depleting agent is an anti-CD20×CD3 bispecific antibody, administering the immunoglobulin depleting agent after the B cell depleting agent will prevent more rapid clearance of the B cell depleting agent. The timing can be affected by what immunoglobulin depleting agent is used. For example, different treatment regimens would be expected for FcRn blockers vs. IgG degrading enzymes (e.g., IdeS). IdeS is an enzyme and therefore acts much more rapidly than FcRn blockade, clearing IgGs within hours to days. For FcRn blockade, several weeks of treatment may be required to fully clear anti-AAV IgGs from circulation.

In some embodiments, a plasma cell depleting agent and/or a B cell depleting agent and/or an immunoglobulin depleting agent and an immunogen (e.g., an immunogenic delivery vehicle) may be administered separately over a defined time course. In certain embodiments, multiple doses of a plasma cell depleting agent and/or a B cell depleting agent and/or an immunoglobulin depleting agent and/or an immunogen (e.g., nucleic acid construct, nuclease agent or CRISPR/Cas system, e.g., in an immunogenic delivery vehicle) (e.g., an immunogenic delivery vehicle) described herein may be administered to a subject over a defined time course. The methods according to such aspects of the disclosure may comprise sequentially administering to a subject multiple doses of a plasma cell depleting agent and/or a B cell depleting agent and/or an immunoglobulin depleting agent and/or immunogen (e.g., nucleic acid construct, nuclease agent or CRISPR/Cas system, e.g., in an immunogenic delivery vehicle) of the disclosure. In some embodiments, when the plasma cell depleting agent and/or the B cell depleting agent and/or the immunoglobulin depleting agent and immunogen (e.g., nucleic acid construct, nuclease agent or CRISPR/Cas system, e.g., in an immunogenic delivery vehicle) are administered sequentially, the plasma cell depleting agent and/or the B cell depleting agent and/or the immunoglobulin depleting agent can be administered before and/or in between each of the administrations of the immunogen(s) (e.g., nucleic acid construct, nuclease agent or CRISPR/Cas system, e.g., in an immunogenic delivery vehicle). As used herein, “sequentially administering” means that each dose of the plasma cell depleting agent, the B cell depleting agent, the immunoglobulin depleting agent, and/or the immunogenic delivery vehicle, is administered to the subject at a different point in time, e.g., on different days separated by a predetermined interval (e.g., hours, days, weeks, months, or years). In some embodiments, the methods of the disclosure comprise sequentially administering to the subject a single initial dose of the plasma cell depleting agent and/or the B cell depleting agent and/or the immunoglobulin depleting agent and/or the immunogen (e.g., nucleic acid construct, nuclease agent or CRISPR/Cas system, e.g., in an immunogenic delivery vehicle), followed by one or more secondary doses of the plasma cell depleting agent and/or the B cell depleting agent and/or the immunoglobulin depleting agent and/or the immunogen (e.g., nucleic acid construct, nuclease agent or CRISPR/Cas system, e.g., in an immunogenic delivery vehicle) (e.g., an immunogenic delivery vehicle), and optionally followed by one or more tertiary doses of the plasma cell depleting agent and/or the B cell depleting agent and/or the immunoglobulin depleting agent and/or the immunogen (e.g., nucleic acid construct, nuclease agent or CRISPR/Cas system, e.g., in an immunogenic delivery vehicle).

The terms “initial dose,” “secondary dose(s),” and “tertiary dose(s)” refer to the temporal sequence of administration of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle) (e.g., an immunogenic delivery vehicle). Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. In some embodiments, the initial, secondary, and tertiary doses may all contain the same amount of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle) but may differ from one another in terms of frequency of administration. In some embodiments, the amount of the plasma cell depleting agent, B cell depleting agent, immunoglobulin depleting agent, and/or the immunogen (e.g., nucleic acid construct, nuclease agent or CRISPR/Cas system, e.g., in an immunogenic delivery vehicle) contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial dose and the one or more secondary doses each contain the same amount of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle). In other embodiments, the initial dose comprises a first amount of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle) (e.g., an immunogenic delivery vehicle) and the one or more secondary doses each comprise a second amount of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle). For example, the first amount of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle) (e.g., an immunogenic delivery vehicle) can be 1.5×, 2×, 2.5×, 3×, 3.5×, 4×, 5× or more than the second amount of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of the plasma cell depleting agent, the B cell depleting agent, the immunoglobulin depleting agent, and/or the immunogen (e.g., nucleic acid construct, nuclease agent or CRISPR/Cas system, e.g., in an immunogenic delivery vehicle) (e.g., an immunogenic delivery vehicle) that is administered to a subject prior to the administration of the very next dose in the sequence with no intervening doses.

The methods of the disclosure may comprise administering to a subject any number of secondary and/or tertiary doses of a plasma cell depleting agent, a B cell depleting agent, an immunoglobulin depleting agent, and/or an immunogen (e.g., nucleic acid construct, nuclease agent or CRISPR/Cas system, e.g., in an immunogenic delivery vehicle) (e.g., an immunogenic delivery vehicle). For example, in certain embodiments, only a single secondary dose is administered to the subject. In other embodiments, two or more (e.g., 2, 3, 4, 5, 6, 7, 8, or more) secondary doses are administered to the subject. Likewise, in certain embodiments, only a single tertiary dose is administered to the subject. In other embodiments, two or more (e.g., 2, 3, 4, 5, 6, 7, 8, or more) tertiary doses are administered to the subject.

In some embodiments involving multiple secondary doses, each secondary dose is administered at the same frequency as the other secondary doses. For example, each secondary dose may be administered to the subject 1, 2, 3, or 4 weeks after the immediately preceding dose. Similarly, in some embodiments involving multiple tertiary doses, each tertiary dose is administered at the same frequency as the other tertiary doses. Alternatively, the frequency at which the secondary and/or tertiary doses are administered to a subject can vary over the course of the treatment regimen. The frequency of administration may also be adjusted during the course of treatment by a physician depending on the needs of the individual subject following clinical examination.

In some embodiments, the secondary and/or tertiary doses of an immunogen (e.g., nucleic acid construct, nuclease agent or CRISPR/Cas system, e.g., in an immunogenic delivery vehicle) (e.g., an immunogenic delivery vehicle) comprising a viral particle or vector (e.g., a viral vector such as an AAV vector) administered to the subject is of the same or similar viral origin as the initial dose. In some embodiments, the secondary and/or tertiary doses of an immunogen (e.g., nucleic acid construct, nuclease agent or CRISPR/Cas system, e.g., in an immunogenic delivery vehicle) (e.g., an immunogenic delivery vehicle) comprising a viral particle or vector administered to the subject is of a different viral origin then the initial dose.

In some embodiments, the subsequently administered viral vector is administered via the same administration route as the originally administered viral vector. In some embodiments, the subsequently administered viral vector is administered via a different administration route from the originally administered viral vector.

In some embodiments, when a plasma cell depleting agent and/or a B cell depleting agent and/or an immunoglobulin depleting agent and/or an immunogen (e.g., nucleic acid construct, nuclease agent or CRISPR/Cas system, e.g., in an immunogenic delivery vehicle) (e.g., an immunogenic delivery vehicle) herein are sequentially administered, the immunogen (e.g., nucleic acid construct, nuclease agent or CRISPR/Cas system, e.g., in an immunogenic delivery vehicle) (e.g., an immunogenic delivery vehicle) may be administered as a first component of the dosing regimen and the plasma cell depleting agent, B cell depleting agent, and/or immunoglobulin depleting agent may be administered as a second component of the dosing regimen (i.e., the immunogen (e.g., nucleic acid construct, nuclease agent or CRISPR/Cas system, e.g., in an immunogenic delivery vehicle) may be administered before the plasma cell depleting agent and/or the B cell depleting agent and/or the immunoglobulin depleting agent). In some embodiments, the immunogen (e.g., nucleic acid construct, nuclease agent or CRISPR/Cas system, e.g., in an immunogenic delivery vehicle) may be administered as a second component of the dosing regimen and the plasma cell depleting agent and/or the B cell depleting agent and/or the immunoglobulin depleting agent may be administered as a first component of a dosing regimen (i.e., the immunogen (e.g., nucleic acid construct, nuclease agent or CRISPR/Cas system, e.g., in an immunogenic delivery vehicle) may be administered after the plasma cell depleting agent and/or the B cell depleting agent and/or the immunoglobulin depleting agent). In some embodiments, an immunogen (e.g., nucleic acid construct, nuclease agent or CRISPR/Cas system, e.g., in an immunogenic delivery vehicle) and a plasma cell depleting agent and/or a B cell depleting agent and/or an immunoglobulin depleting agent may be sequentially administered, in either of the above-described orders, with variable time intervals between administration. For example, the time interval between administration of the immunogen (e.g., nucleic acid construct, nuclease agent or CRISPR/Cas system, e.g., in an immunogenic delivery vehicle) (e.g., an immunogenic delivery vehicle) and the plasma cell depleting agent and/or B cell depleting agent and/or immunoglobulin depleting agent may be at least about 30 seconds, at least about 35 seconds, at least about 40 seconds, at least about 45 seconds, at least about 50 seconds, at least about 55 seconds, at least about 1 minute, at least about 2 minutes, at least about 5 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 10 to 12 hours, at least about 12 to 14 hours, at least about 14 to 16 hours, at least about 16 to 18 hours, at least about 18 to 20 hours, at least about 20 to 22 hours, at least about 22 to 24 hours, at least about 1 day, at least about 2 days, at least about 3 days, at least about 4 days, at least about 5 days, at least about 6 days, at least about 7 days, at least about 8 days, at least about 9 days, at least about 10 days, at least about 10 to 12 days, at least about 12 to 14 days, at least about 14 to 16 days, at least about 16 to 18 days, at least about 18 to 20 days, at least about 20 to 22 days, at least about 22 to 24 days, at least about 24 to 26 days, at least about 28 days, at least about 29 days, at least about 30 days, at least about 31 days, at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 2 years, at least about 3 years, at least about 4 years, at least about 5 years, at least about 6 years, at least about 7 years, at least about 8 years, at least about 9 years, at least about 10 years, or more.

Any of the above methods can further comprise any of various subsequent administration steps described herein. The subsequent administration step can comprise, for example, administering the plasma cell depleting agent and/or the B cell depleting agent and/or the immunoglobulin depleting agent and an immunogen (e.g., nucleic acid construct, nuclease agent or CRISPR/Cas system, e.g., in an immunogenic delivery vehicle) (e.g., an immunogenic delivery vehicle) to the subject one or more subsequent times until a desired level of expression and/or activity of the polypeptide of interest is achieved in the subject.

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

The subsequent administration step can comprise, for example, administering a second immunogen (e.g., nucleic acid construct, nuclease agent or CRISPR/Cas system, e.g., in an immunogenic delivery vehicle) (e.g., an immunogenic delivery vehicle), e.g. an immune delivery vehicle comprising, e.g., a vector comprising a coding sequence for a second polypeptide of interest (e.g., that is different from a first polypeptide of interest encoded by a first vector administered in an initial administration step) to the subject one or more subsequent times until a desired level of expression and/or activity of the polypeptide of interest is achieved in the subject.

The subsequent administration step can be, for example, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, or at least about 12 weeks after the initial dosing (e.g., at least about 4 weeks after the initial dosing) or about 4 weeks to about 12 weeks, about 4 weeks to about 13 weeks, about 4 weeks to about 14 weeks, about 4 weeks to about 15 weeks, about 4 weeks to about 16 weeks, about 1 week to about 12 weeks, about 2 weeks to about 12 weeks, about 3 weeks to about 12 weeks, about 1 week to about 15 weeks, about 2 week to about 14 weeks, or about 3 weeks to about 13 weeks after the initial dosing (e.g., about 4 weeks to about 12 weeks after the initial dosing).

In some embodiments, the present disclosure provides a method for increasing effectiveness of a subsequently administered viral vector following an originally administered viral vector in a subject in need thereof, comprising administering to the subject an effective amount of a plasma cell depleting agent, a B cell depleting agent, and/or an immunoglobulin depleting agent, and the subsequently administered viral vector is of the same or similar viral origin as the originally administered viral vector. In some embodiments, the present disclosure contemplates a method for increasing effectiveness of a subsequently administered viral vector following an originally administered viral vector in a subject in need thereof, comprising administering to the subject an effective amount of a plasma cell depleting agent, a B cell depleting agent, and/or an immunoglobulin depleting agent, and the subsequently administered viral vector is of the same or similar viral origin as the originally administered viral vector. In another aspect, provided herein is a method for increasing effectiveness of a subsequently administered viral vector following an originally administered viral vector in a subject in need thereof, the method comprising administering to the subject an effective amount of an anti-CD20×CD3 bispecific antibody or a functional fragment thereof, wherein the subsequently administered viral vector is of the same or similar viral origin as the originally administered viral vector. In some embodiments, the subject does not have a pre-existing immunity against the viral vectors. In some embodiments, the subject does not have a pre-existing immunity against the nucleic acid construct, the polypeptide of interest encoded by the nucleic acid construct, the nuclease agent or one or more nucleic acids encoding the nuclease agent, or a delivery vehicle for the nucleic acid construct, the nuclease agent, or the one or more nucleic acids encoding the nuclease agent.

In some embodiments, the subsequently administered viral vector is administered via the same administration route as the originally administered viral vector.

In some embodiments, the subsequently administered viral vector is administered via a different administration route from the originally administered viral vector.

In some embodiments, the plasma cell depleting agent is administered before the administration of the subsequently administered viral vector(s).

In some embodiments, the plasma cell depleting agent is administered simultaneously with the administration of the subsequently administered viral vector(s).

In some embodiments, the subsequently administered viral vectors are administered two or more times and the plasma cell depleting agent is administered before and/or between each of the administrations of the subsequently administered viral vectors.

In some embodiments, the plasma cell depleting agent, the B cell depleting agent, and/or the immunoglobulin depleting agent is administered before the administration of the viral vector(s). In some embodiments, the anti-CD20×CD3 bispecific antibody or functional fragment thereof is administered before the administration of the originally administered viral vector to the subject.

In some embodiments, the plasma cell depleting agent, the B cell depleting agent, and/or the immunoglobulin depleting agent is administered simultaneously with the administration of the viral vector(s). In some embodiments, the anti-CD20×CD3 bispecific antibody or functional fragment thereof is administered simultaneously with the administration of the originally administered viral vector and/or subsequently administered viral vector to the subject.

In some embodiments, the plasma cell depleting agent, the B cell depleting agent, and/or the immunoglobulin depleting agent is administered after the administration of the viral vector(s). In some embodiments, the anti-CD20×CD3 bispecific antibody or functional fragment thereof is administered after the administration of the originally administered viral vector but before administering the subsequently administered viral vector to the subject. In some embodiments, the anti-CD20×CD3 bispecific antibody or functional fragment thereof is administered after the administration of the subsequently administered viral vector to the subject.

In some embodiments, the viral vectors are administered two or more times and the plasma cell depleting agent, the B cell depleting agent, and/or the immunoglobulin depleting agent is administered before and/or between each of the administrations of the viral vectors. In some embodiments, the anti-CD20×CD3 bispecific antibody or functional fragment thereof is administered before and/or between each of the administrations of the viral vectors to the subject.

In some embodiments, the immunogen re-administration occurs via the same administration route as its prior administration. In some embodiments, the immunogen re-administration occurs via a different administration route than its prior administration. In some embodiments, the plasma cell depleting agent, the B cell depleting agent, and/or the immunoglobulin depleting agent is administered before the administration of the immunogen. In some embodiments, the anti-CD20×CD3 bispecific antibody or functional fragment thereof is administered before the administration of the immunogen to the subject. In some embodiments, the anti-CD20×CD3 bispecific antibody or functional fragment thereof is administered before the administration of the immunogenic delivery vehicle to the subject. In some embodiments, the plasma cell depleting agent, the B cell depleting agent, and/or the immunoglobulin depleting agent is administered simultaneously with the administration of the immunogen. In some embodiments, the anti-CD20×CD3 bispecific antibody or functional fragment thereof is administered simultaneously with the administration of the immunogen to the subject. In some embodiments, the anti-CD20×CD3 bispecific antibody or functional fragment thereof is administered simultaneously with the administration of the immunogenic delivery vehicle to the subject. In some embodiments, the plasma cell depleting agent, the B cell depleting agent, and/or the immunoglobulin depleting agent is administered after the administration of the immunogen. In some embodiments, the anti-CD20×CD3 bispecific antibody or functional fragment thereof is administered after the administration of the immunogen to the subject. In some embodiments, the anti-CD20×CD3 bispecific antibody or functional fragment thereof is administered after the administration of the immunogenic delivery vehicle to the subject. In some embodiments, the immunogen is administered two or more times and the plasma cell depleting agent, the B cell depleting agent, and/or the immunoglobulin depleting agent is administered before and/or between each of the administrations of the immunogen. In some embodiments, the immunogen is administered to the subject two or more times and the anti-CD20×CD3 bispecific antibody or functional fragment thereof is administered before and/or between each of the administrations of the immunogen. In some embodiments, the immunogenic delivery vehicle is administered to the subject two or more times and the anti-CD20×CD3 bispecific antibody or functional fragment thereof is administered before and/or between each of the administrations of the immunogenic delivery vehicle. In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen, e.g., 2-4 days afterwards as plasma cell formation may be limited during the initial lag period. In some embodiments, such as when the immunogen is administered two or more times, the plasma cell depleting agent is administered before and/or between each of the administrations of the immunogen. Administration of the plasma cell depleting agent shortly after the administration of the immunogen may prevent plasma cell formation and persistence elicited by administration of the immunogen to immunologically naïve patients. In some embodiments (e.g., if the patient is immunologically naïve), the B cell depleting agent is administered simultaneously with the administration of the immunogen (e.g., to prevent any B cells from persisting after being formed). In some embodiments, the B cell depleting agent is administered after the administration of the immunogen (e.g., 2-4 days afterwards as B cell formation may be limited during the initial lag period). Administration of the B cell depleting agent shortly after the administration of the immunogen may prevent B cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In any of the above methods, the plasma cell depleting agent and/or the B cell depleting agent and/or the immunoglobulin depleting agent can be administered simultaneously with the immunogen (e.g., nucleic acid construct, nuclease agent or CRISPR/Cas system, e.g., in an immunogenic delivery vehicle) (e.g., an immunogenic delivery vehicle) or not simultaneously (e.g., sequentially in any combination). For example, in a method comprising administering a composition comprising a plasma cell depleting agent and/or a B cell depleting agent and/or an immunoglobulin depleting agent and an immunogen (e.g., nucleic acid construct, nuclease agent or CRISPR/Cas system, e.g., in an immunogenic delivery vehicle), they can be administered separately (e.g., the plasma cell depleting agent and/or the B cell depleting agent and/or the immunoglobulin depleting agent separately from the immunogen (e.g., nucleic acid construct, nuclease agent or CRISPR/Cas system, e.g., in an immunogenic delivery vehicle)). For example, the plasma cell depleting agent and/or B cell depleting agent and/or immunoglobulin depleting agent can be administered prior to the immunogen (e.g., nucleic acid construct, nuclease agent or CRISPR/Cas system, e.g., in an immunogenic delivery vehicle), subsequent to the immunogen (e.g., nucleic acid construct, nuclease agent or CRISPR/Cas system, e.g., in an immunogenic delivery vehicle), prior to and subsequent to the immunogen (e.g., nucleic acid construct, nuclease agent or CRISPR/Cas system, e.g., in an immunogenic delivery vehicle), or at the same time as immunogen (e.g., nucleic acid construct, nuclease agent or CRISPR/Cas system, e.g., in an immunogenic delivery vehicle). In some embodiments, the plasma cell depleting agent is administered after an immune response has been developed. In some embodiments (e.g., if the patient is immunologically naïve), the plasma cell depleting agent is administered simultaneously with the administration of the immunogen (e.g., to prevent any plasma cells from persisting after being formed). In some embodiments, the plasma cell depleting agent is administered after the administration of the immunogen (e.g., 2-4 days afterwards as plasma cell formation may be limited during the initial lag period). Administration of the plasma cell depleting agent shortly after the administration of the immunogen may prevent plasma cell formation and persistence elicited by administration of the immunogen to immunologically naïve patients. In some embodiments (e.g., if the patient already has pre-existing immunity), the plasma cell depleting agent is administered before the administration of the immunogen. In some embodiments (e.g., if the patient already has pre-existing immunity), the plasma cell depleting agent is administered again within a short period of the first administration. In some embodiments (e.g., if the patient already has pre-existing immunity), the plasma cell depleting agent is continuously administered throughout the pre-dose and re-dose periods (e.g., to clear plasma cells and keep plasma cell levels low). In some embodiments (e.g., if the patient already has pre-existing immunity), the plasma cell depleting agent is administered prophylactically.

In some embodiments, the plasma cell depleting agent and/or the B cell depleting agent and/or the immunoglobulin depleting agent can be administered about 1 hour to about 48 hours, about 1 hour to about 24 hours, about 1 hour to about 12 hours, about 1 hour to about 6 hours, about 1 hour to about 2 hours, about 2 hours to about 48 hours, about 2 hours to about 24 hours, about 2 hours to about 12 hours, about 2 hours to about 6 hours, about 3 hours to about 48 hours, about 6 hours to about 48 hours, about 12 hours to about 48 hours, or about 24 hours to about 48 hours prior to and/or subsequent to administration of the immunogen (e.g., nucleic acid construct, nuclease agent or CRISPR/Cas system, e.g., in an immunogenic delivery vehicle).

In one example, the plasma cell depleting agent and/or B cell depleting agent and/or immunoglobulin depleting agent is administered about 4 hours, about 8 hours, about 12 hours, about 18 hours, about 1 day, about 2 days, about 3 days, about 4 days, about 5 days, about 6 days, or about 1 week prior to administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent and/or B cell depleting agent and/or immunoglobulin depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent and/or B cell depleting agent and/or immunoglobulin depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and/or subsequent to administering the immunogen (e.g., nucleic acid construct, nuclease agent or CRISPR/Cas system, e.g., in an immunogenic delivery vehicle) (e.g., an immunogenic delivery vehicle).

In one example, the plasma cell depleting agent and/or the B cell depleting agent and/or the immunoglobulin depleting agent is administered about 4 hours, about 8 hours, about 12 hours, about 18 hours, about 1 day, about 2 days, about 3 days, about 4 days, about 5 days, about 6 days, or about 1 week prior to and subsequent to administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent and/or the B cell depleting agent and/or the immunoglobulin depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week prior to and subsequent to administering immunogen (e.g., nucleic acid construct, nuclease agent or CRISPR/Cas system, e.g., in an immunogenic delivery vehicle) (e.g., an immunogenic delivery vehicle). In another example, the plasma cell depleting agent and/or the B cell depleting agent and/or the immunoglobulin depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days prior to and subsequent to administering the immunogen (e.g., nucleic acid construct, nuclease agent or CRISPR/Cas system, e.g., in an immunogenic delivery vehicle) (e.g., an immunogenic delivery vehicle).

In one example, the plasma cell depleting agent and/or B cell depleting agent and/or immunoglobulin depleting agent is administered about 4 hours, about 8 hours, about 12 hours, about 18 hours, about 1 day, about 2 days, about 3 days, about 4 days, about 5 days, about 6 days, or about 1 week after administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent and/or B cell depleting agent and/or immunoglobulin depleting agent is administered at least about 4 hours, at least about 8 hours, at least about 12 hours, at least about 18 hours, at least about 1 day, at least about 2 days, at least about 3 days, at least about 4 days, at least about 5 days, at least about 6 days, or at least about 1 week after administering the immunogen (e.g., nucleic acid construct, nuclease agent or CRISPR/Cas system, e.g., in an immunogenic delivery vehicle) (e.g., an immunogenic delivery vehicle). In another example, the plasma cell depleting agent and/or B cell depleting agent and/or immunoglobulin depleting agent is administered about 4 hours to about 24 hours, about 4 hours to about 12 hours, about 4 hours to about 8 hours, about 8 hours to about 24 hours, about 12 hours to about 24 hours, about 1 day to about 7 days, about 1 day to about 6 days, about 1 day to about 5 days, about 1 day to about 4 days, about 1 day to about 3 days, about 1 day to about 2 days, about 2 days to about 7 days, about 3 days to about 7 days, about 4 days to about 7 days, about 5 days to about 7 days, about 6 days to about 7 days, or about 1 day to about 3 days after administering the immunogen (e.g., nucleic acid construct, nuclease agent or CRISPR/Cas system, e.g., in an immunogenic delivery vehicle) (e.g., an immunogenic delivery vehicle).

In one example, the plasma cell depleting agent and/or B cell depleting agent and/or immunoglobulin depleting agent (e.g., the plasma cell depleting agent) is administered within about 6 months after the nucleic acid construct, optionally wherein the nucleic acid construct is in a viral vector, and the plasma cell depleting agent is administered if the viral vector is still present in the subject. In one example, the immunogen (e.g., nucleic acid construct or AAV) is administered within about 3 months, within about 2 months, within about 7 weeks, within about 6 weeks, within about 5 weeks, within about 4 weeks, within about 3 weeks, or within about 2 weeks after an initial dose of the plasma cell depleting agent and/or B cell depleting agent and/or immunoglobulin depleting agent (e.g., the plasma cell depleting agent). In one example, the immunogen (e.g., nucleic acid construct or AAV) is administered at least about 2 weeks, at least about 3 weeks, at least about 4 weeks, at least about 5 weeks, at least about 6 weeks, at least about 7 weeks, at least about 2 months, or at least about 3 months after an initial dose of the plasma cell depleting agent and/or B cell depleting agent and/or immunoglobulin depleting agent (e.g., the plasma cell depleting agent). In one example, the immunogen (e.g., nucleic acid construct or AAV) is administered within about 2 weeks to about 3 months, within about 2 weeks to about 2 months, within about 2 weeks to about 7 weeks, within about 2 weeks to about 6 weeks, within about 2 weeks to about 5 weeks, within about 2 weeks to about 4 weeks, within about 2 weeks to about 3 weeks, within about 2 months to about 3 months, within about 7 weeks to about 3 months, within about 6 weeks to about 3 months, within about 5 weeks to about 3 months, within about 4 weeks to about 3 months, within about 3 weeks to about 3 months, or within about 2 weeks to about 3 months after an initial dose of the plasma cell depleting agent and/or B cell depleting agent and/or immunoglobulin depleting agent (e.g., the plasma cell depleting agent). In one example, the immunogen (e.g., nucleic acid construct or AAV) is administered within about 2 weeks to about 7 weeks, within about 3 weeks to about 6 weeks, within about 4 weeks to about 5 weeks, within about 3 weeks to about 7 weeks, within about 4 weeks to about 7 weeks, within about 5 weeks to about 7 weeks, within about 2 weeks to about 6 weeks, within about 2 weeks to about 5 weeks, within about 2 weeks to about 4 weeks, or within about 4 weeks to about 6 weeks after an initial dose of the plasma cell depleting agent and/or B cell depleting agent and/or immunoglobulin depleting agent (e.g., the plasma cell depleting agent). The timing can depend, for example, on initial starting titer, and which IgG clearing agent is used. In some embodiments, for FcRn blockers, for low titers, the timing may be about 2 days to about 1 week. In some embodiments, for FcRn blockers, for high titers, it the timing may be about 4 to about 7 weeks. In some embodiments, for IgG degrading enzymes, for IdeS, the timing may be about 1 week to about 4 weeks for plasma cell depleting agent, then about 2 days to about 1 week for IdeS.

In one example in immunologically naïve subjects, the immunogen (e.g., nucleic acid construct or AAV) is administered within about 3 months, within about 2 months, within about 7 weeks, within about 6 weeks, within about 5 weeks, within about 4 weeks, within about 3 weeks, within about 2 weeks, or within about 1 week after an initial dose of the B cell depleting agent. In one example, the immunogen (e.g., nucleic acid construct or AAV) is administered at least about 1 week, at least about 2 weeks, at least about 3 weeks, at least about 4 weeks, at least about 5 weeks, at least about 6 weeks, at least about 7 weeks, at least about 2 months, or at least about 3 months after an initial dose of the B cell depleting agent. In one example, the immunogen (e.g., nucleic acid construct or AAV) is administered within about 1 week to about 3 months, within about 1 week to about 2 months, within about 1 week to about 7 weeks, within about 1 week to about 6 weeks, within about 1 week to about 5 weeks, within about 1 week to about 4 weeks, within about 1 week to about 3 weeks, within about 1 week to about 2 weeks, within about 2 months to about 3 months, within about 7 weeks to about 3 months, within about 6 weeks to about 3 months, within about 5 weeks to about 3 months, within about 4 weeks to about 3 months, within about 3 weeks to about 3 months, or within about 2 weeks to about 3 months after an initial dose of the B cell depleting agent. In one example, the immunogen (e.g., nucleic acid construct or AAV) is administered within about 1 week to about 7 weeks, within about 2 weeks to about 6 weeks, within about 3 weeks to about 5 weeks, within about 3 weeks to about 7 weeks, within about 4 weeks to about 7 weeks, within about 5 weeks to about 7 weeks, within about 1 week to about 6 weeks, within about 1 week to about 5 weeks, within about 1 week to about 4 weeks, or within about 1 week to about 2 weeks after an initial dose of the B cell depleting agent. In one example, the immunogen (e.g., nucleic acid construct or AAV) is administered within about 1 week before and within about 1 week after the B cell depleting agent.

Administration in vivo can be by any suitable route including, for example, parenteral, intravenous, oral, subcutaneous, intra-arterial, intracranial, intrathecal, intratumoral, intraperitoneal, topical, intranasal, or intramuscular. Systemic modes of administration include, for example, oral and parenteral routes. Examples of parenteral routes include intravenous, intraarterial, intraosseous, intramuscular, intradermal, subcutaneous, intranasal, and intraperitoneal routes. A specific example is intravenous infusion. Nasal instillation and intravitreal injection are other specific examples. Local modes of administration include, for example, intrathecal, intracerebroventricular, intraparenchymal (e.g., localized intraparenchymal delivery to the striatum (e.g., into the caudate or into the putamen), cerebral cortex, precentral gyrus, hippocampus (e.g., into the dentate gyrus or CA3 region), temporal cortex, amygdala, frontal cortex, thalamus, cerebellum, medulla, hypothalamus, tectum, tegmentum, or substantia nigra), intraocular, intraorbital, subconjunctival, intravitreal, subretinal, and transscleral routes. Significantly smaller amounts of the components (compared with systemic approaches) may exert an effect when administered locally (for example, intraparenchymal or intravitreal) compared to when administered systemically (for example, intravenously). Local modes of administration may also reduce or eliminate the incidence of potentially toxic side effects that may occur when therapeutically effective amounts of a component are administered systemically. In a specific example, administration in vivo is intravenous.

Administration in vivo can be by any suitable route including, for example, parenteral, intravenous, oral, subcutaneous, intra-arterial, intracranial, intrathecal, intraperitoneal, intratumoral, topical, intranasal, or intramuscular. A specific example is intravenous infusion.

Administration in vivo can be by any suitable route including, for example, systemic routes of administration such as parenteral administration, e.g., intravenous, subcutaneous, intra-arterial, or intramuscular. In a specific example, administration in vivo is intravenous.

The frequency of administration and the number of dosages can depend on a number of factors. The introduction of an immunogen (e.g., nucleic acid construct, nuclease agent or CRISPR/Cas system, e.g., in an immunogenic delivery vehicle) into the cell or subject can be performed one time or multiple times over a period of time. For example, the introduction can be performed only once over a period of time, at least two times over a period of time, at least three times over a period of time, at least four times over a period of time, at least five times over a period of time, at least six times over a period of time, at least seven times over a period of time, at least eight times over a period of time, at least nine times over a period of times, at least ten times over a period of time, at least eleven times, at least twelve times over a period of time, at least thirteen times over a period of time, at least fourteen times over a period of time, at least fifteen times over a period of time, at least sixteen times over a period of time, at least seventeen times over a period of time, at least eighteen times over a period of time, at least nineteen times over a period of time, or at least twenty times over a period of time. In some methods, a single administration of the immunogen (e.g., nucleic acid construct, nuclease agent or CRISPR/Cas system, e.g., in an immunogenic delivery vehicle), e.g., a vector, is sufficient to increase expression of polypeptide of interest to a desirable level. In other methods, more than one administration may be beneficial to maximize therapeutic effect.

The methods disclosed herein can increase polypeptide of interest protein levels and/or polypeptide of interest activity levels in a cell or subject (e.g., circulating, serum, or plasma levels in a subject) and can comprise measuring polypeptide of interest protein levels and/or polypeptide of interest activity levels in a cell or subject (e.g., circulating, serum, or plasma levels in a subject).

In some methods, polypeptide of interest activity and/or expression levels in a subject are increased to about or at least about 2%, about or at least about 10%, about or at least about 25%, about or at least about 40%, about or at least about 50%, about or at least about 75%, or at least about 100%, or more, of normal level. In some methods, polypeptide of interest activity and/or expression levels in a subject are increased to about or at least about 40%, about or at least about 50%, about or at least about 75%, or at least about 100%, or more, of normal level.

In some methods, circulating polypeptide of interest levels (i.e., serum levels) are about or at least about 0.5, about or at least about 1, about or at least about 2, about or at least about 3, about or at least about 4, about or at least about 5, about or at least about 6, about or at least about 7, about or at least about 8, about or at least about 9, or about or at least about 10 μg/mL. In some methods, polypeptide of interest levels are at least about 1 μg/mL or about 1 μg/mL. In some methods, polypeptide of interest levels are at least about 2 μg/mL or about 2 μg/mL. In some methods, polypeptide of interest levels are at least about 5 μg/mL or about 5 μg/mL. In some methods, polypeptide of interest levels are about 1 μg/mL to about 30 μg/mL, about 2 μg/mL to about 30 μg/mL, about 3 μg/mL to about 30 μg/mL, about 4 μg/mL to about 30 μg/mL, about 5 μg/mL to about 30 μg/mL, about 1 μg/mL to about 20 μg/mL, about 2 μg/mL to about 20 μg/mL, about 3 μg/mL to about 20 μg/mL, about 4 μg/mL to about 20 μg/mL, about 5 μg/mL to about 20 μg/mL. For example, the method can result in polypeptide of interest levels of about 2 μg/mL to about 30 μg/mL or 2 μg/mL to about 20 μg/mL. For example, the method can result in polypeptide of interest levels of about 5 μg/mL to about 30 μg/mL or 5 μg/mL to about 20 μg/mL. In some embodiments, the recited expression levels are at least 1 month after administration. In some embodiments, the recited expression levels are at least 2 months after administration. In some embodiments, the recited expression levels are at least 3 months after administration. In some embodiments, the recited expression levels are at least 4 months after administration. In some embodiments, the recited expression levels are at least 5 months after administration. In some embodiments, the recited expression levels are at least 6 months after administration. In some embodiments, the recited expression levels are at least 9 months after administration. In some embodiments, the recited expression levels are at least 12 months after administration.

In some methods, the method increases expression and/or activity of polypeptide of interest over the subject's baseline expression and/or activity (i.e., expression and/or activity prior to administration). In some methods, the method increases expression and/or activity of polypeptide of interest over the subject's baseline expression and/or activity (i.e., expression and/or activity prior to administration. In some methods, polypeptide of interest activity and/or polypeptide of interest expression or serum levels in a subject are increased by about or at least about 10%, about or at least about 25%, about or at least about 50%, about or at least about 75%, or about or at least about 100%, or more, as compared to the subject's polypeptide of interest expression or serum levels and/or activity before administration (i.e., the subject's baseline levels). In certain embodiments, the loss of function is nearly complete such that a relative activity cannot be determined. In certain embodiments, the level of expression is sufficient to treat at least one sign or symptom resulting from the loss of function of the polypeptide of interest.

In some methods, the method increases expression and/or activity of the polypeptide of interest over the cell's baseline expression and/or activity (i.e., expression and/or activity prior to administration). In some methods, the method increases expression and/or activity of polypeptide of interest over the cell's baseline expression and/or activity (i.e., expression and/or activity prior to administration. In some methods, polypeptide of interest activity and/or expression levels in a cell or population of cells (e.g., liver cells, or hepatocytes) are increased by about or at least about 10%, about or at least about 25%, about or at least about 50%, about or at least about 75%, about or at least about 100%, or more, as compared to the polypeptide of interest activity and/or expression levels before administration (i.e., the subject's baseline levels). In certain embodiments, the polypeptide of interest loss of function is nearly complete such that a relative activity cannot be determined. In certain embodiments, the level of expression is sufficient to treat at least one sign or symptom resulting from the loss of function of the polypeptide of interest.

In a specific example, the polypeptide of interest activity levels in a subject are increased to no more than about 300%, no more than about 250%, no more than about 200%, or no more than about 150% of normal polypeptide of interest activity levels.

In a specific example, the polypeptide of interest activity levels in the subject are increased to at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels. In a specific example, the polypeptide of interest activity levels in the subject are increased to at least about 40%, at least about 45%, at least about 50%, at least about 55%, at least about 60%, at least about 65%, at least about 70%, at least about 75%, at least about 80%, at least about 85%, at least about 90%, or at least about 100% of normal polypeptide of interest activity levels.

In some methods, the method results in increased expression of the polypeptide of interest in the subject (e.g., neonatal subject) compared to a method comprising administering an episomal expression vector encoding the polypeptide of interest in a control subject. In some methods, the method results in increased serum levels of the polypeptide of interest in the subject (e.g., neonatal subject) compared to a method comprising administering an episomal expression vector encoding the polypeptide of interest to a control subject.

In some methods, the method increases expression or activity of the polypeptide of interest over the subject's baseline expression or activity of the polypeptide of interest (i.e., any percent change in expression that is larger than typical error bars). In some methods, the method results in expression of the polypeptide of interest at a detectable level above zero, e.g., at a statistically significant level, a clinically relevant level.

In some methods, the expression or activity of the polypeptide of interest is at least 50% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at one year after the administering. In some methods, the expression or activity of the polypeptide of interest is at least 60% of the expression or activity of the polypeptide of interest at a peak level of expression measured for the subject at 24 weeks after the administering. In some methods, the expression or activity of the polypeptide of interest is at least 50% of the expression or activity of the polypeptide of interest at a peak level of expression measured for the subject at two years after the administering. In some methods, the expression or activity of the polypeptide of interest is at least 60% of the expression or activity of the polypeptide of interest at a peak level of expression measured for the subject at 2 years after the administering. In some methods, the expression or activity of the polypeptide of interest is at least 60% of the expression or activity of the polypeptide of interest at a peak level of expression measured for the subject at 24 weeks after the administering.

In some methods, the method further comprises assessing preexisting anti-polypeptide of interest immunity in a subject prior to administering any of the nucleic acid constructs described herein. For example, such methods could comprise assessing immunogenicity using a total antibody (TAb) immune assay or a neutralizing antibody (NAb) assay. In some methods, the subject has not previously been administered recombinant polypeptide of interest protein. In some methods, the subject has previously been administered recombinant polypeptide of interest protein.

In some methods, the method further comprises determining whether the subject has immunity against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent prior to the administering of any of the above. For example, the determining can comprise determining the presence of neutralizing antibodies against the nucleic acid construct, the polypeptide of interest, the nuclease agent, the one or more nucleic acids encoding the nuclease agent, or the delivery vehicle for the nucleic acid construct, the nuclease agent, or the one or more nucleic acids encoding the nuclease agent (e.g., determining the presence of neutralizing antibodies against an AAV comprising a nucleic acid construct). For example, the determining the presence of neutralizing antibodies can comprise determining whether there is an effective level of neutralizing antibody to prevent the intended outcome of insertion of a nucleic acid construct into a genomic locus or expression of the polypeptide of interest encoded by the nucleic acid construct. In some methods, the method further comprises assessing preexisting anti-AAV (e.g., anti-AAV8) immunity in a subject prior to administering any of the nucleic acid constructs described herein. For example, such methods could comprise assessing immunogenicity using a total antibody (TAb) immune assay or a neutralizing antibody (NAb) assay. See, e.g., Manno et al. (2006) Nat. Med. 12(3):342-347, Kruzik et al. (2019) Mol. Ther. Methods Clin. Dev. 14:126-133, and Weber (2021) Front. Immunol. 12:658399, each of which is herein incorporated by reference in its entirety for all purposes. In some embodiments, TAb assays look for antibodies that bind to the AAV vector, whereas NAb assays assess whether the antibodies that are present stop the AAV vector from transducing target cells. With TAb assays, the drug product or an empty capsid can be used to capture the antibodies; NAb assays can require a reporter vector (e.g., a version of the AAV vector encoding luciferase). In some embodiments, the subject does not have preexisting anti-AAV immunity. In some embodiments, the subject does have preexisting AAV immunity.

### XVIII. Kits

The present disclosure further comprises a kit which may comprise any of various compositions of the present disclosure, including the plasma cell depleting agents, the B cell depleting agents, the immunoglobulin depleting agents, and/or the immunogens (e.g., nucleic acid construct, nuclease agent or CRISPR/Cas system, e.g., in an immunogenic delivery vehicle) (e.g., immunogenic delivery vehicles), or pharmaceutical compositions thereof, of the disclosure.

One exemplary embodiment of the present disclosure comprises a kit comprising (i) a plasma cell depleting agent, (ii) a B cell depleting agent and/or an immunoglobulin depleting agent, and (iii) optionally, instructions for use. Another exemplary embodiment of the present disclosure comprises a kit comprising (i) an immunogen, (ii) a plasma cell depleting agent, (iii) optionally a B cell depleting agent and/or an immunoglobulin depleting agent, and (iv) optionally, instructions for use. Yet another exemplary embodiment of the present disclosure comprises a kit comprising (i) an immunogen, (ii) an anti-CD20×CD3 bispecific antibody or a functional fragment thereof, and (iii) optionally, instructions for use.

In one aspect, the present disclosure may include a kit comprising, for example: (a) a container that contains a pharmaceutical composition disclosed herein, for example, a pharmaceutical composition in solution or in lyophilized form; (b) optionally, a second container containing a diluent or reconstituting solution for the lyophilized formulation; and/or (c) optionally, instructions for (i) use of the solution or (ii) reconstitution and/or use of the lyophilized formulation.

In some embodiments, the kit may further comprise, for example, without limitation, one or more of (i) a buffer, (ii) a diluent, (iii) a filter, (iv) a needle, and/or (v) a syringe. As a non-limiting example, the container may be a bottle, a vial, a syringe, or test tube. In some embodiments, the container may be a multi-use container. In some the pharmaceutical composition may be lyophilized.

Kits of the present disclosure may comprise a lyophilized formulation of the present disclosure in a suitable container and instructions for its reconstitution and/or use. Suitable containers include, for example, bottles, vials (e.g., dual chamber vials), syringes (such as dual chamber syringes) and test tubes. The container may be formed from a variety of materials such as glass or plastic. The kit and/or container may contain instructions on or associated with the container that indicate directions for reconstitution of the lyophilized formulation and/or use of the kit. For example, the label may indicate that the lyophilized formulation is to be reconstituted to an appropriate peptide concentration. The label may indicate that the formulation is useful or intended for any route of administration disclosed herein, e.g., parenteral administration routes disclosed herein.

The container holding the formulation may be a multi-use vial, which may allow for repeat administrations (e.g., from 2-6 administrations) of the reconstituted formulation. The kit may further comprise a second container comprising a suitable diluent (e.g., sodium bicarbonate solution).

Upon mixing of the diluent and the lyophilized formulation, the final peptide concentration in the reconstituted formulation is reached. The kit may further include other materials desirable from a commercial and/or user standpoint, including, for example, without limitation, other buffers, diluents, filters, needles, syringes, and/or package inserts which may comprise, e.g., instructions for use.

Kits of the present disclosure may have a single container that contains the formulation of the pharmaceutical compositions according to the present disclosure with or without other components (e.g., other compounds or pharmaceutical compositions of these other compounds) or may have a distinct container for each component.

In some embodiments, kits of the disclosure may include a formulation of the disclosure packaged for use in combination with the coadministration of a second compound (such as adjuvants (e.g., GM-CSF, a chemotherapeutic agent, a natural product, a hormone or antagonist, an anti-angiogenesis agent or inhibitor, an apoptosis-inducing agent, or a chelator) or a pharmaceutical composition thereof. The components of the kit may be pre-complexed or each component may be in a separate distinct container prior to administration to a patient. The components of the kit may be provided in one or more liquid solutions. A liquid solution described herein may be an aqueous solution, for example, a sterile aqueous solution. The components of the kit may also be provided as solids, which may be converted into liquids such as by addition of suitable solvents, which may be provided in another distinct container.

The container of a therapeutic kit may be a vial, test tube, flask, bottle, syringe, or any other means of enclosing a solid or liquid. When there is more than one component, the kit may contain a second vial or other container, which may allow for separate dosing. The kit may also contain another container for a pharmaceutically acceptable liquid. In some embodiment, a kit may contain an apparatus (e.g., one or more needles, syringes, eye droppers, pipettes, etc.), which may allow for administration of the agents of the disclosure that are components of the present kit.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### BRIEF DESCRIPTION OF THE SEQUENCES

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. When a nucleotide sequence encoding an amino acid sequence is provided, it is understood that codon degenerate variants thereof that encode the same amino acid sequence are also provided. The amino acid sequences follow the standard convention of beginning at the amino terminus of the sequence and proceeding forward (i.e., from left to right in each line) to the carboxy terminus.

TABLE 12
Description of Sequences.

| SEQ ID NO | Type | Description |
|---|---|---|
| 1 | DNA | Anti-BCMA HCVR DNA Sequence |
| 2 | Protein | Anti-BCMA HCVR Protein Sequence |
| 3 | DNA | Anti-BCMA HCDR1 DNA Sequence |
| 4 | Protein | Anti-BCMA HCDR1 Protein Sequence |
| 5 | DNA | Anti-BCMA HCDR2 DNA Sequence |
| 6 | Protein | Anti-BCMA HCDR2 Protein Sequence |
| 7 | DNA | Anti-BCMA HCDR3 DNA Sequence |
| 8 | Protein | Anti-BCMA HCDR3 Protein Sequence |
| 9 | DNA | Anti-BCMA LCVR DNA Sequence |
| 10 | Protein | Anti-BCMA LCVR Protein Sequence |
| 11 | DNA | Anti-BCMA LCDR1 DNA Sequence |
| 12 | Protein | Anti-BCMA LCDR1 Protein Sequence |
| 13 | DNA | Anti-BCMA LCDR2 DNA Sequence |
| 14 | Protein | Anti-BCMA LCDR2 Protein Sequence |
| 15 | DNA | Anti-BCMA LCDR3 DNA Sequence |
| 16 | Protein | Anti-BCMA LCDR3 Protein Sequence |
| 17 | DNA | Common LCVR DNA Sequence |
| 18 | Protein | Common LCVR Protein Sequence |
| 19 | DNA | Common LCDR1 DNA Sequence |
| 29 | Protein | Common LCDR1 Protein Sequence |
| 21 | DNA | Common LCDR2 DNA Sequence |
| 22 | Protein | Common LCDR2 Protein Sequence |
| 23 | DNA | Common LCDR3 DNA Sequence |
| 24 | Protein | Common LCDR3 Protein Sequence |
| 25 | DNA | Anti-CD3 HCVR DNA Sequence - REGN5458 |
| 26 | Protein | Anti-CD3 HCVR Protein Sequence - REGN5458 |
| 27 | DNA | Anti-CD3 HCDR1 DNA Sequence - REGN5458 |
| 28 | Protein | Anti-CD3 HCDR1 Protein Sequence - REGN5458 |
| 29 | DNA | Anti-CD3 HCDR2 DNA Sequence - REGN5458 |
| 30 | Protein | Anti-CD3 HCDR2 Protein Sequence - REGN5458 |
| 31 | DNA | Anti-CD3 HCDR3 DNA Sequence - REGN5458 |
| 32 | Protein | Anti-CD3 HCDR3 Protein Sequence - REGN5458 |
| 33 | DNA | Anti-CD3 HCVR DNA Sequence - REGN5459 |
| 34 | Protein | Anti-CD3 HCVR Protein Sequence - REGN5459 |
| 35 | DNA | Anti-CD3 HCDR1 DNA Sequence - REGN5459 |
| 36 | Protein | Anti-CD3 HCDR1 Protein Sequence - REGN5459 |
| 37 | DNA | Anti-CD3 HCDR2 DNA Sequence - REGN5459 |
| 38 | Protein | Anti-CD3 HCDR2 Protein Sequence - REGN5459 |
| 39 | DNA | Anti-CD3 HCDR3 DNA Sequence - REGN5459 |
| 40 | Protein | Anti-CD3 HCDR3 Protein Sequence - REGN5459 |
| 41 | Protein | Anti-BCMA Heavy Chain Protein Sequence (IgG4 Heavy Chain Constant Region) |
| 42 | Protein | Anti-CD3 Heavy Chain Protein Sequence (IgG4 Heavy Chain Constant Region with |
| | | H435R/Y436F) |
| 43 | Protein | Common Anti-BCMA and Anti-CD3 Light Chain Protein Sequence (Kappa Light |
| | | Chain Constant Region) |
| 44 | Protein | Anti-CD20 HCVR Protein Sequence |
| 45 | Protein | Common LCVR Protein Sequence |
| 46 | Protein | Anti-CD3 HCVR Protein Sequence |
| 47 | Protein | Anti-CD20 HCDR1 Protein Sequence |
| 48 | Protein | Anti-CD20 HCDR2 Protein Sequence |
| 49 | Protein | Anti-CD20 HCDR3 Protein Sequence |
| 50 | Protein | Common LCDR1 Protein Sequence |
| 51 | Protein | Common LCDR2 Protein Sequence |
| 52 | Protein | Common LCDR3 Protein Sequence |
| 53 | Protein | Anti-CD3 HCDR1 Protein Sequence |
| 54 | Protein | Anti-CD3 HCDR2 Protein Sequence |
| 55 | Protein | Anti-CD3 HCDR3 Protein Sequence |
| 56 | Blank | Blank |
| 57 | Protein | Human Factor IX Protein NCBI Accession No. NP_000124.1 |
| 58 | DNA | Human F9 mRNA (cDNA) NCBI Accession No. NM_000133.4 |
| 59 | DNA | Human F9 CDS CCDS ID CCDS14666.1 |
| 60 | DNA | Native F9 Insert |
| 61 | DNA | Native CpG removed no splice F9 Insert |
| 62 | DNA | Native CpG removed F9 Insert |
| 63 | DNA | Native splice removed F9 Insert |
| 64 | DNA | Codon optimized F9 Insert |
| 65 | DNA | COMP F9 Insert |
| 66 | DNA | DC F9 Insert |
| 67 | DNA | GA F9 Insert |
| 68 | DNA | CpG0 F9 Insert |
| 69 | DNA | CpG3 F9 Insert |
| 70 | DNA | CpG10 no CpG F9 Insert |
| 71 | DNA | CpG10 F9 Insert |
| 72 | DNA | CpG20 no CpG F9 Insert |
| 73 | DNA | CpG20 F9 Insert |
| 74 | DNA | Insert 72 |
| 75 | DNA | Insert 18 |
| 76 | DNA | Insert 19 |
| 77 | DNA | Insert 20 |
| 78 | DNA | Insert 21 |
| 79 | DNA | Insert 27 |
| 80 | DNA | Insert 28 |
| 81 | DNA | Insert 29 |
| 82 | DNA | Insert 30 |
| 83 | DNA | Insert 36 |
| 84 | DNA | Insert 37 |
| 85 | DNA | Insert 38 |
| 86 | DNA | Insert 39 |
| 87 | DNA | Insert 22 |
| 88 | DNA | Insert 23 |
| 89 | DNA | Insert 24 |
| 90 | DNA | Insert 25 |
| 91 | DNA | Insert 26 |
| 92 | DNA | Insert 31 |
| 93 | DNA | Insert 32 |
| 94 | DNA | Insert 33 |
| 95 | DNA | Insert 34 |
| 96 | DNA | Insert 35 |
| 97 | Protein | FIX Encoded by F9 Inserts |
| 98 | DNA | SV40 polyA |
| 99 | DNA | CpG depleted bGH polyA |
| 100 | DNA | Mouse Alb exon 2 Splice Acceptor |
| 101 | DNA | Insert 72 no ITRs |
| 102 | DNA | Insert 18 no ITRs |
| 103 | DNA | Insert 19 no ITRs |
| 104 | DNA | Insert 20 no ITRs |
| 105 | DNA | Insert 21 no ITRs |
| 106 | DNA | Insert 27 no ITRs |
| 107 | DNA | Insert 28 no ITRs |
| 108 | DNA | Insert 29 no ITRs |
| 109 | DNA | Insert 30 no ITRs |
| 110 | DNA | Insert 36 no ITRs |
| 111 | DNA | Insert 37 no ITRs |
| 112 | DNA | Insert 38 no ITRs |
| 113 | DNA | Insert 39 no ITRs |
| 114 | DNA | Insert 22 no ITRs |
| 115 | DNA | Insert 23 no ITRs |
| 116 | DNA | Insert 24 no ITRs |
| 117 | DNA | Insert 25 no ITRs |
| 118 | DNA | Insert 26 no ITRs |
| 119 | DNA | Insert 31 no ITRs |
| 120 | DNA | Insert 32 no ITRs |
| 121 | DNA | Insert 33 no ITRs |
| 122 | DNA | Insert 34 no ITRs |
| 123 | DNA | Insert 35 no ITRs |
| 124 | RNA | Cas9 mRNA |
| 125 | RNA | Cas9 mRNA CDS |
| 126 | DNA | Cas9 CDS |
| 127 | DNA | Human ALB Intron 1 |
| 128 | DNA | Guide RNA Target Sequence Plus PAM v1 |
| 129 | DNA | Guide RNA Target Sequence Plus PAM v2 |
| 130 | DNA | Guide RNA Target Sequence Plus PAM v3 |
| 131 | Protein | SpCas9 Protein V1 |
| 132 | DNA | SpCas9 DNA V1 |
| 133 | DNA | SpCas9 mRNA (cDNA) |
| 134 | Protein | SpCas9 Protein V2 |
| 135 | RNA | SpCas9 mRNA V2 |
| 136 | Protein | SV40 NLS v1 |
| 137 | Protein | SV40 NLS v2 |
| 138 | Protein | Nucleoplasmin NLS |
| 139 | RNA | crRNA Tail v1 |
| 140 | RNA | crRNA Tail v2 |
| 141 | RNA | TracrRNA v1 |
| 142 | RNA | TracrRNA v2 |
| 143 | RNA | TracrRNA v3 |
| 144 | RNA | gRNA Scaffold v1 |
| 145 | RNA | gRNA Scaffold v2 |
| 146 | RNA | gRNA Scaffold v3 |
| 147 | RNA | gRNA Scaffold v4 |
| 148 | RNA | gRNA Scaffold v5 |
| 149 | RNA | gRNA Scaffold v6 |
| 150 | RNA | gRNA Scaffold v7 |
| 151 | RNA | gRNA Scaffold v8 |
| 152 | RNA | Modified gRNA Scaffold |
| 153-184 | RNA | Human ALB Intron 1 Guide Sequences |
| 185-248 | RNA | Human ALB Intron 1 sgRNA Sequences |
| 249-280 | DNA | Human ALB Intron 1 Guide RNA Target Sequences |
| 281 | DNA | ITR 145 |
| 282 | DNA | ITR 141 |
| 283 | DNA | ITR 130 |
| 284 | DNA | SV40 polyA |
| 285 | DNA | bGH polyA |
| 286 | DNA | Mouse Alb exon 2 Splice Acceptor |
| 287 | RNA | Mouse Alb Intron 1 Guide Sequence g666 |
| 288 | DNA | Mouse Alb Intron 1 Guide RNA Target Sequence g666 |
| 289-290 | RNA | Mouse Alb Intron 1 sgRNA Sequences g666 |
| 291 | DNA | ITR 145 Reverse Complement |
| 292 | DNA | SV40 polyA v2 |
| 293 | Protein | Human GAA Protein (NP_000143.2) |
| 294 | DNA | Human GAA cDNA/mRNA (NM_000152.5) |
| 295 | DNA | Human GAA CDS (CCDS32760.1) |
| 296 | Protein | Human GAA (70-952) Protein |
| 297 | DNA | Human GAA (70-952) CDS |
| 298 | DNA | Human GAA (70-952) CDS - DC-0 |
| 299 | DNA | Human GAA (70-952) CDS - GA-0 |
| 300 | DNA | Human GAA (70-952) CDS - GS-0 |
| 301 | DNA | Human GAA (70-952) CDS - GS-0v2 |
| 302 | DNA | Human GAA (70-952) CDS - GS-1 |
| 303 | DNA | Human GAA (70-952) CDS - GS-44 |
| 304 | DNA | Human GAA (70-952) CDS - GS-50 |
| 305 | DNA | Human GAA (70-952) CDS - RE-8 |
| 306 | Protein | 12450 anti-CD63 scFv |
| 307 | DNA | 12450 anti-CD63 scFv CDS |
| 308 | DNA | 12450 anti-CD63 scFv CDS - DC-0 |
| 309 | DNA | 12450 anti-CD63 scFv CDS - GA-0 |
| 310 | DNA | 12450 anti-CD63 scFv CDS - GS-0 |
| 311 | DNA | 12450 anti-CD63 scFv CDS - GS-0v2 |
| 312 | DNA | 12450 anti-CD63 scFv CDS - GS-1 |
| 313 | DNA | 12450 anti-CD63 scFv CDS - GS-44 |
| 314 | DNA | 12450 anti-CD63 scFv CDS - GS-50 |
| 315 | DNA | 12450 anti-CD63 scFv CDS - RE-8 |
| 316 | Protein | 12450 anti-CD63 scFv:GAA (70-952) Fusion Protein |
| 317 | DNA | 12450 anti-CD63 scFv:GAA (70-952) Fusion Protein CDS |
| 318 | DNA | 12450 anti-CD63 scFv:GAA (70-952) Fusion Protein CDS - DC-0 |
| 319 | DNA | 12450 anti-CD63 scFv:GAA (70-952) Fusion Protein CDS - GA-0 |
| 320 | DNA | 12450 anti-CD63 scFv:GAA (70-952) Fusion Protein CDS - GS-0 |
| 321 | DNA | 12450 anti-CD63 scFv:GAA (70-952) Fusion Protein CDS - GS-0v2 |
| 322 | DNA | 12450 anti-CD63 scFv:GAA (70-952) Fusion Protein CDS - GS-1 |
| 323 | DNA | 12450 anti-CD63 scFv:GAA (70-952) Fusion Protein CDS - GS-44 |
| 324 | DNA | 12450 anti-CD63 scFv:GAA (70-952) Fusion Protein CDS - GS-50 |
| 325 | DNA | 12450 anti-CD63 scFv:GAA (70-952) Fusion Protein CDS - RE-8 |
| 326 | DNA | Human GAA (70-952) CDS - 1 - DC |
| 327 | DNA | Human GAA (70-952) CDS - 1 - GS |
| 328 | DNA | Human GAA (70-952) CDS - 2 - DC |
| 329 | DNA | Human GAA (70-952) CDS - 2 - GS |
| 330 | DNA | Human GAA (70-952) CDS - 3 - DC |
| 331 | DNA | Human GAA (70-952) CDS - 3 - GS |
| 332 | DNA | Human GAA (70-952) CDS - 4 - DC |
| 333 | DNA | Human GAA (70-952) CDS - 4 - GS |
| 334-655 | DNA & | Domains in Anti-hTfR Antibodies, Antigen-Binding Fragments or scFv Molecules |
| | Protein | |
| 656-687 | Protein | Anti-hTfR scFvs |
| 688 | Protein | 12799B-2xG4S-GAA |
| 689 | Protein | 12839B-2xG4S-GAA |
| 690 | Protein | 12843B-2xG4S-GAA |
| 691 | Protein | 12847B-2xG4S-GAA |
| 692 | DNA | 12799B-2xG4S-GAA |
| 693 | DNA | 12839B-2xG4S-GAA |
| 694 | DNA | 12843B-2xG4S-GAA |
| 695 | DNA | 12847B-2xG4S-GAA |
| 696-698 | DNA | Optimized 12799B-2xG4S-GAA |
| 699-701 | DNA | Optimized 12843B-2xG4S-GAA |
| 702-704 | DNA | Optimized 12847B-2xG4S-GAA |
| 705 | DNA | 12799 GA 0 anti-TfR scFv |
| 706 | DNA | 12799 GS 0 anti-TfR scFv |
| 707 | DNA | 12799 GS 0v2 anti-TfR scFv |
| 708 | DNA | 12843 GA 0 anti-TfR scFv |
| 709 | DNA | 12843 GS 0 anti-TfR scFv |
| 710 | DNA | 12843 GS 0v2 anti-TfR scFv |
| 711 | DNA | 12847 GA 0 anti-TfR scFv |
| 712 | DNA | 12847 GS 0 anti-TfR scFv |
| 713 | DNA | 12847 GS 0v2 anti-TfR scFv |
| 714 | DNA | 12799 anti-TfR scFv |
| 715 | DNA | 12839 anti-TfR scFv |
| 716 | DNA | 12843 anti-TfR scFv |
| 717 | DNA | 12847 anti-TfR scFv |
| 718 | Protein | Linker |
| 719 | Protein | Kappa Constant Light Domain |
| 720 | Protein | IgG1 CH1 Heavy Domain |
| 721-790 | Protein | Fab Heavy and Light Chains |
| 791 | Protein | Mus musculus Ror1 Signal Peptide |
| 792 | Protein | IgG4 CH1 Heavy Domain |
| 793-824 | Protein | Additional anti-TfR scFv:GAA Sequences |
| 825 | DNA | ITR 141 Reverse Complement |
| 826 | DNA | ITR 130 Reverse Complement |
| 827 | DNA | SV40 polyA V3 |
| 828 | Protein | 3X G4S Linker |
| 829 | Protein | 2X G4S Linker |
| 830-834 | DNA | 3X G4S Linker Coding Sequences |
| 835-841 | DNA | 2X G4S Linker Coding Sequences |
| 842 | DNA | 1X G4S Linker Coding Sequence |
| 843 | DNA | pINT ITR130 12843 GAA native SV40pA |
| 844 | DNA | pINT ITR130 12843scFv:GAA 0CpG v0 (GA 0) |
| 845 | DNA | pINT ITR130 12843scFv:GAA 0CpG v1 (GS 0 v1) |
| 846 | DNA | pINT ITR130 12843scFv:GAA 0CpG v2 (GS 0 v2) |
| 847 | DNA | pINT ITR 130 12847scFv:GAA native |
| 848 | DNA | pINT ITR130 12847scFv:GAA 0CpG v0 (GA 0) |
| 849 | DNA | pINT ITR130 12847scFv:GAA 0CpG v1 (GS 0 v1) |
| 850 | DNA | PINT ITR130 12847scFv:GAA 0CpG v2 (GS 0 v2) |
| 851 | DNA | pINT-ITR130-Anti-CD63:GAA GA 0 |
| 852 | DNA | 2xFix 12847 anti-TfR:GAA Coding Sequence |
| 853 | Protein | 12847 anti-TfR:GAA Fusion Protein |
| 854 | DNA | 3xG4S Linker Coding Sequence |
| 855 | DNA | 1xG4S Linker Coding Sequence |
| 856 | DNA | GAA (70-952) Coding Sequence [3078_GT_>_GG] |
| 857 | DNA | GAA (70-952) Coding Sequence [1830_GGTGGT_>_GGGGGC] |
| | | [3078_GT_>_GG] |
| 858 | DNA | bGH polyA v2 |
| 859 | DNA | SV40_late_polyA w/Reverse Strand AAUAAAs Mutated to AAUCAAs |
| 860 | DNA | Synthetic polyA (SPA) |
| 861 | DNA | Stuffer Sequence |
| 862 | DNA | MAZ Element |
| 863 | DNA | 6xFix anti-CD63:GAA Coding Sequence |
| 864 | DNA | 4xFix anti-CD63:GAA Coding Sequence [274_G > T] 723_T > G |
| | | 1830_GGT_GGT_>_GGG_GGC 3078_GT > GG] |
| 865 | DNA | 1xFix anti-CD63:GAA Coding Sequence [3078_GT > GG] |
| 866 | DNA | 6xFix anti-CD63 scFv Coding Sequence |
| 867 | DNA | 4xFix anti-CD63 scFv Coding Sequence |
| 868 | Protein | Anti-CD63 scFv VH |
| 869 | Protein | Anti-CD63 scFv VK |
| 870 | DNA | VVT874/VVT1251 - TfR Original Template pMID27199 ITR to ITR |
| 871 | DNA | VVT1125/VVT1261 - pAAV-12847GAA(GS0v2)-2Xfix-bGH.sv40LuniPA |
| 872 | DNA | VVT1129/VVT1262 - pAAV-12847GAA(GS0v2)-2Xfix-bGHpA |
| 873 | DNA | VVT1126 - pAAV-12847GAA(GS0v2)-bGHpA swap |
| 874 | DNA | VVT1254/VVT1252 - CD63 Original Template pMID27863 ITR to ITR |
| 875 | DNA | VVT1121 - pAAV-CD63GAA-4Xfix-bGH.sv40LuniPA |
| 876 | DNA | VVT1122 - pAAV-CD63GAA-4Xfix-bGHpA |
| 877 | DNA | VVT1123 - pAAV-CD63GAA-3078fix-bGH.sv40LuniPA (1CpG) |
| 878 | DNA | VVT1118 - pAAV-CD63GAA-3078fix-bGHpA |
| 879 | DNA | VVT1124 - pAAV-CD63GAA-bGHpA swap |
| 880 | DNA | VVT1119 - pAAV-CD63GAA-bGHpA.SPA swap |
| 881 | DNA | VVT1120 - pAAV-CD63GAA-bGHpA.stuffer.SPA |
| 882 | DNA | VVT1138 - pAAV-CD63GAA-bGHpA-MAZ |
| 883 | DNA | VVT1127 - pAAV-CD63GAA-SV40E-MAZ |
| 884 | DNA | VVT1128/VVT1263 - pAAV-CD63GAA-6Xfix-bGH.sv40LuniPA |
| 885 | DNA | VVT1139/VVT1264 - pAAV-CD63GAA-6Xfix-bGHpA |
| 886 | DNA | VVT874/VVT1251 - TfR Original Template pMID27199 - no ITRs |
| 887 | DNA | VVT1125VVT/1261 - pAAV-12847GAA(GS0v2)-2Xfix-bGH.sv40LuniPA - no |
| | | ITRs |
| 888 | DNA | VVT1129/VVT1262 - pAAV-12847GAA(GS0v2)-2Xfix-bGHpA - no ITRs |
| 889 | DNA | VVT1126 - pAAV-12847GAA(GS0v2)-bGHpA swap - no ITRs |
| 890 | DNA | VVT1254/VVT1252 - CD63 Original Template pMID27863 - no ITRs |
| 891 | DNA | VVT1121 - pAAV-CD63GAA-4Xfix-bGH.sv40LuniPA - no ITRs |
| 892 | DNA | VVT1122 - pAAV-CD63GAA-4Xfix-bGHpA - no ITRs |
| 893 | DNA | VVT1123 - pAAV-CD63GAA-3078fix-bGH.sv40LuniPA (1CpG) - no ITRs |
| 894 | DNA | VVT1118 - pAAV-CD63GAA-3078fix-bGHpA - no ITRs |
| 895 | DNA | VVT1124 - pAAV-CD63GAA-bGHpA swap - no ITRs |
| 896 | DNA | VVT1119 - pAAV-CD63GAA-bGHpA.SPA swap - no ITRs |
| 897 | DNA | VVT1120 - pAAV-CD63GAA-bGHpA.stuffer.SPA - no ITRs |
| 898 | DNA | VVT1138 - pAAV-CD63GAA-bGHpA-MAZ - no ITRs |
| 899 | DNA | VVT1127 - pAAV-CD63GAA-SV40E-MAZ - no ITRs |
| 900 | DNA | VVT1128/VVT1263 - pAAV-CD63GAA-6Xfix-bGH.sv40LuniPA - no ITRs |
| 901 | DNA | VVT1139/VVT1264 - pAAV-CD63GAA-6Xfix-bGHpA - no ITRs |
| 902 | DNA | Combined BGH and Unidirectional SV40 Late Polyadenylation Signal |

### EXAMPLES

### Example 1. Plasma Cell Depletion in Combination with Neonatal Fc Receptor (FcRn) Blockade Elicits Rapid and Deep Suppression of Pre-Existing Andi-AAV Antibody Titers

For adeno-associated virus gene therapy, the generation of neutralizing antibodies after exposure precludes the ability to re-administer AAV vectors of the same or related serotypes, despite therapeutic need. Furthermore, due to natural exposure to wild-type AAVs, roughly 30-60% of individuals harbor pre-existing antibodies to AAV that prevent administration of even a single AAV vector. Therefore, strategies that can attenuate pre-existing anti-AAV antibody responses induced by either recombinant or wild type AAVs have the potential to greatly expand the versatility and accessibility of AAV gene therapies to a broader patient population. In some embodiments, the subsequently administered AAV vector has a capsid derived from the same AAV serotype as the originally administered AAV vector.

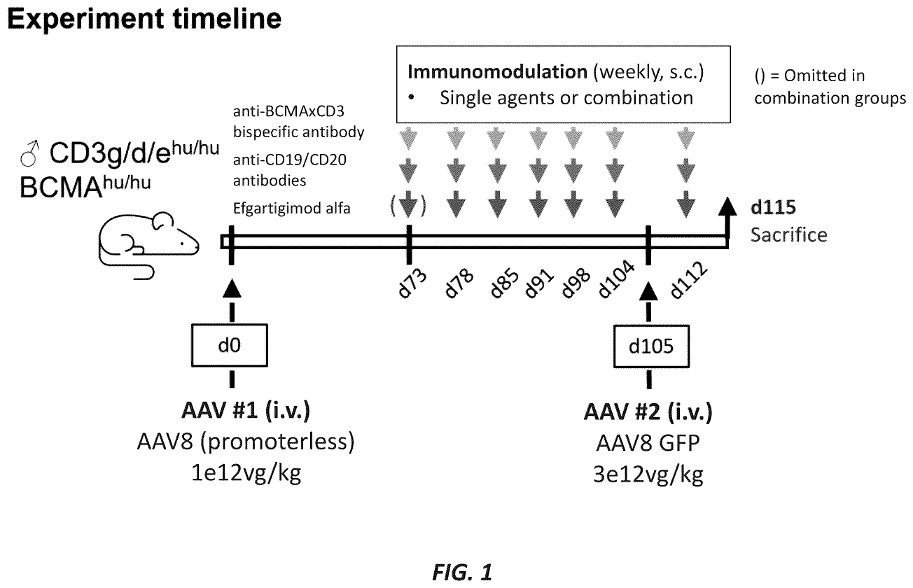

Because plasma cells are the source of long-lived antibody responses, it was reasoned that antibody-mediated plasma cell depletion may suppress pre-existing antibody responses to AAV sufficiently to enable AAV vector transduction or re-transduction in seropositive animals. To test this, B cell maturation antigen (BCMA)- and CD3 gamma-, CD3 delta-, and CD3 epsilon-humanized mice (n=6 per group) were treated with 1e12 vector genomes (vg) per kilogram (kg) recombinant AAV8 (encoding a promoterless transgene) to induce a strong anti-capsid antibody response (e.g., high-titer nAbs). 73 days later, a timepoint deemed sufficient to account for long-lived plasma cell differentiation, mice were injected subcutaneously weekly for five weeks with 25 milligrams (mg) per kg linvoseltamab, also known as REGN5458, a fully-human T cell-bridging bispecific antibody targeting B cell maturation antigen and CD3 (referred to herein as “anti-BCMA×CD3 bispecific antibody”) to induce plasma cell depletion. Additionally, because the half-life of immunoglobulin G is relatively long (˜6-8 days in mice and ˜21 days in humans) due to the action of neonatal Fc receptor (“FcRn”), it was also evaluated whether additional blockade of FcRn with efgartigimod alfa, administered subcutaneously weekly at 20 mg/kg, could further accelerate and improve titer reductions elicited by plasma cell depletion. Finally, to capture a wider range of AAV-specific B cells that may not express high levels of BCMA, such as committed memory B cells and early plasmablasts, it was also tested whether additional B cell depletion with a cocktail of anti-CD19/CD20 antibodies, administered subcutaneously weekly at 25 mg/kg each, may further improve the therapeutic effect of plasma cell depletion with anti-BCMA×CD3 bispecific antibody. Mice were bled at defined intervals for serum anti-AAV antibody analysis. A schematic of the full experimental design is presented in FIG. 1. To prevent any initial impact of efgartigimod alfa on the therapeutic effect of anti-BCMA×CD3 bispecific antibody or anti-CD19/CD20 antibodies, which are themselves immunoglobulins, efgartigimod alfa was omitted from the first week's treatment cocktails.

To evaluate the impact of plasma cell depletion, FcRn blockade, B cell depletion, or combinations thereof on anti-AAV8 IgG titers, anti-capsid IgG levels were measured in serum of mice over time. Specifically, 96 well flat-bottom plates were coated with 1e9 vg/well recombinant AAV8 vector in DPBS overnight. The next day, plates were washed and blocked with 0.5% bovine serum albumin in DPBS for 1 hr. Serum samples were then diluted 3×, beginning at an initial dilution of 1:300 and ending at a dilution of 53,144,100. Diluted serum was then transferred to the assay plate and incubated overnight at 4° C. The next day, the assay plates were repeatedly washed prior to incubation with an anti-mouse-IgG Fc-gamma Fragment-HRP-conjugated polyclonal secondary antibody (Jackson Immunoresearch, West Grove, PA). Plates were again repeatedly washed prior to development with TMB substrate solution. After 20 minutes, the reaction was stopped by addition of 2N phosphoric acid. Absorbance at 450 nm (OD450) was measured on a SpectraMax i3 plate reader (Molecular Devices, San Jose, CA). Relative levels of serum anti-AAV8 IgG were determined and plotted as titer values using Prism v.9 software (GraphPad, Boston, MA). Titer was defined as the dilution factor required to achieve an OD450 reading equal to 2-fold higher than background values.

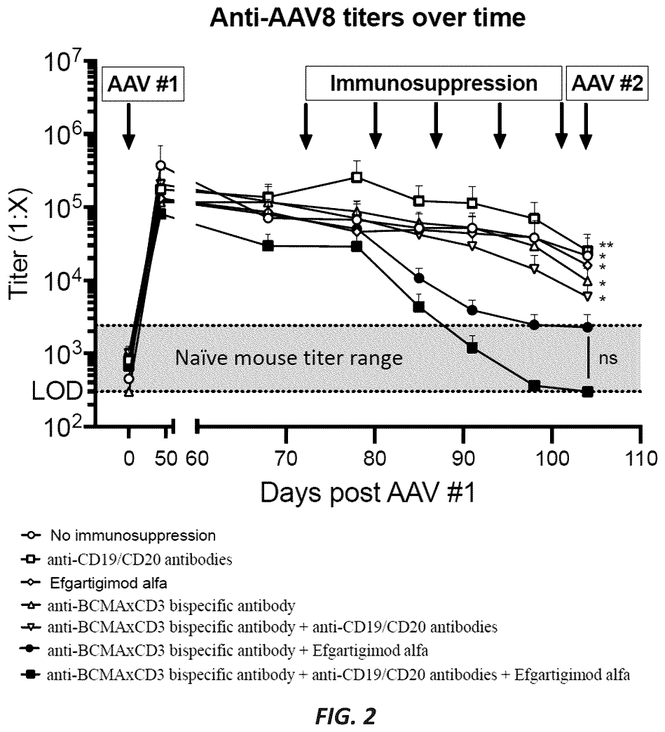

It was found that while anti-AAV8 antibody titers showed minor declines over time in mice administered either anti-BCMA×CD3 bispecific antibody, efgartigimod alfa, or anti-CD19/CD20 antibodies individually, the titer reductions were minor and not statistically different from AAV-treated mice that received no immunomodulation. By contrast, mice receiving a cocktail of anti-BCMA×CD3 bispecific antibody and efgartigimod alfa showed rapid titer declines to naïve or near-naive levels, and mice additionally treated with anti-CD19/CD20 antibodies showed even more rapid and complete titer declines, with all 6/6 mice exhibiting titers below the limit of detection by the cessation of the five-week treatment period (FIG. 2). Thus, these data demonstrate that anti-AAV8 titers can be suppressed by therapeutic plasma cell depletion, with the timeframe required for anti-AAV8 titer depletion reduced by FcRn blockade. Additionally, the depth of titer reduction can be further enhanced with B cell depletion in mice.

### Example 2. Plasma Cell Depletion in Combination with FcRn Blockade Enables AAV Vector Re-Administration

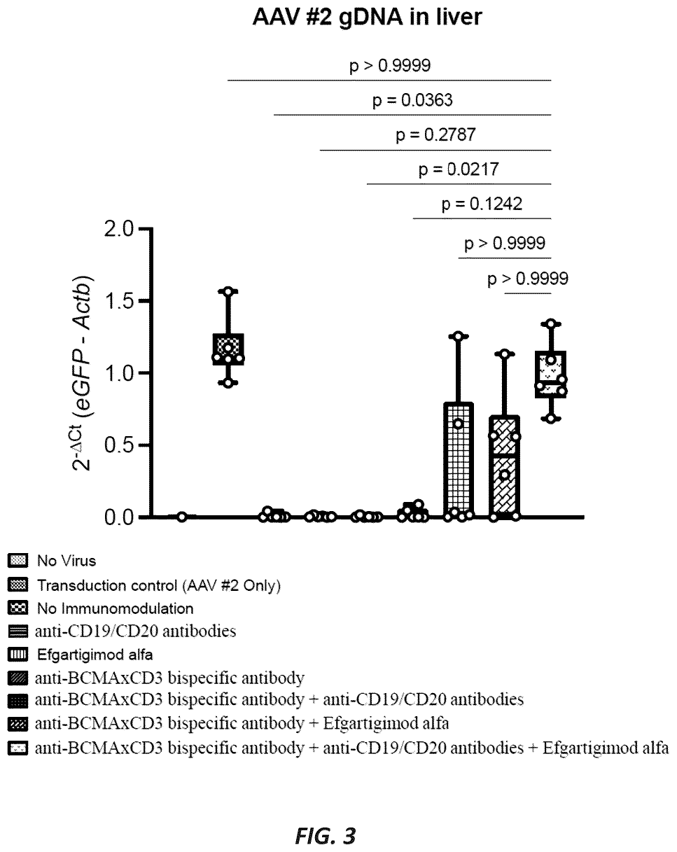

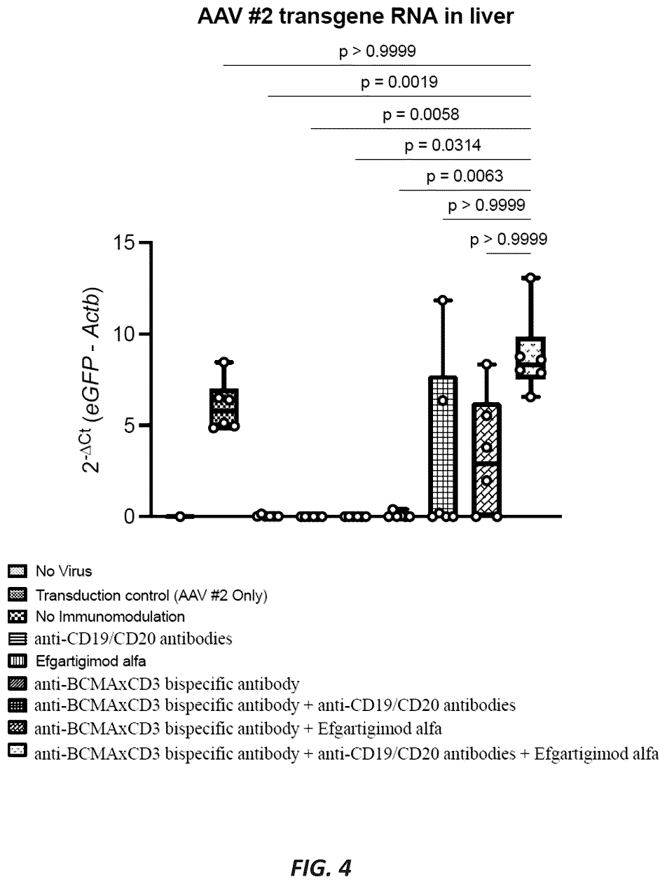

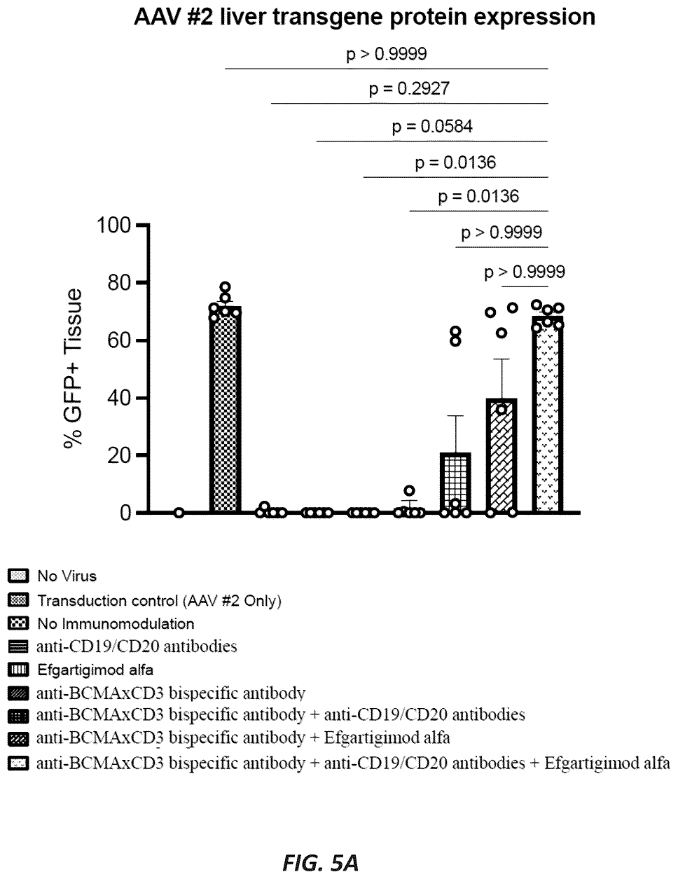

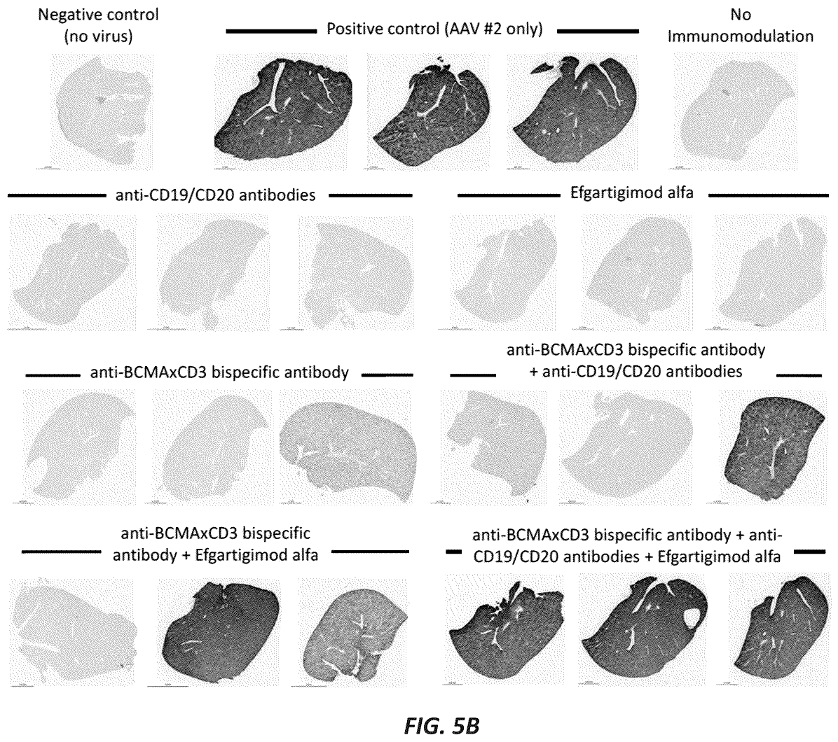

Next was evaluated whether the deep titer reductions observed in mice following combination treatment of anti-BCMA×CD3 bispecific antibody and efgartigimod alfa, and following combination treatment of anti-BCMA×CD3 bispecific antibody, efgartigimod alfa, and anti-CD19/CD20 antibodies, could enable re-transduction with a second AAV vector. To this end, the mice from Example 1 were treated intravenously with 3e12 vg/kg AAV8 GFP, then sacrificed 10 days later to evaluate transgene expression in liver (FIG. 1). While nearly all mice receiving no immunomodulation or single-agent immunomodulation failed to achieve any re-transduction in liver, due to presence of anti-AAV8 antibodies from previous AAV8 exposure, significant levels of GFP transgene DNA (FIG. 3) and RNA (FIG. 4) were observed in mice receiving anti-BCMA×CD3 bispecific antibody+FcRn blocker, and less frequently in mice receiving anti-BCMA×CD3 bispecific antibody+anti-CD19/CD20 antibodies, as measured by quantitative real-time PCR and quantitative real-time reverse transcription PCR, respectively. 3/6 mice receiving anti-BCMA×CD3 bispecific antibody+FcRn blocker achieved re-transduction equivalent to seronegative control mice. The greatest level of re-transduction was observed in the triple combination group, with 6/6 mice achieving transgene levels equivalent to that of previously naive mice. Inmunohistochemical staining of formalin-fixed, paraffin-embedded liver sections for GFP transgene protein corroborated these findings (FIGS. 5A-5B). Together, these data indicate that anti-BCMA×CD3 bispecific antibody-mediated plasma cell depletion, particularly in combination with FcRn blockade, can enable AAV re-dosing, or other immunogenic gene therapy vectors, regardless of serostatus, and that the success rate for re-dosing may be further enhanced by B cell depletion in mice.

### Example 3. Analysis of Plasma Cell Frequencies and Counts in Spleen and Bone Marrow Following Anti-BCMA×CD3 Bispecific Antibody Treatment

To confirm on-target activity of anti-BCMA×CD3 bispecific antibody, plasma cell numbers in bone marrow and spleen were evaluated by flow cytometry at the time of sacrifice (FIG. 1). Specifically, single-cell splenocyte suspensions were prepared by mechanical disruption of spleen. For bone marrow extraction, femurs were cut at both ends, placed in a PCR plate with holes punched at the bottom, and spun down for 3 minutes at 500 g. Red blood cells were lysed using ACK lysis buffer. Cells were transferred to a 96 well U-bottom plate, centrifuged at 400 g for 4 minutes and stained with LIVE/DEAD Fixable Blue Dead Cell dye (ThermoFisher) for 15 minutes at room temperature. Cells were washed and incubated in Fc block (Tonbo Biosciences) for 15 to 30 minutes at 4° C. For detection of AAV-specific B cells, splenocytes were incubated with recombinant AAV8 for 1 hr on ice, (multiplicity of infection [MOI] of 10,000) to facilitate interactions between AAV particles and antigen-specific BCRs, followed by washing and labeling with anti-AAV8 biotinylated antibody (clone ADK8, Progen) and surface stain antibody cocktail (Table 13) for 30 minutes at 4° C. in Brilliant Stain buffer (BD Biosciences). Cells were again washed and then stained with Streptavidin-PE conjugate (Biolegend) for an additional 20 minutes at 4° C., followed by washing and fixation with BD Cytofix (BD Biosciences). For intracellular staining, samples were washed and incubated in 1× Perm/Wash buffer (BD Biosciences) for 20 minutes and resuspended in intracellular stain (Table 13) for 30 minutes at 4° C. followed by washing and fixation with BD Cytofix (BD Biosciences). CountBright Absolute counting Beads (ThermoFisher) were used according to the manufacturer's protocol to enumerate absolute cell counts. Acquisition was performed on a RD FACSymphony A5 using FACSDiva software. Analysis was performed using FlowJo or OMIQ software. All B cells were first gated according to light scatter properties, then negatively gated to exclude viability dye positive and non-B cell lineage marker positive cells. Specific B cell populations were then gated as follows: Naïve B cells, CD19+B220+CD1d−IgD+CD38+; Memory B cells, CD19+B220+CD1d−IgD−CD38+AAV+/−; Plasma cells: B220−IgD-CD138+Light Chain+.

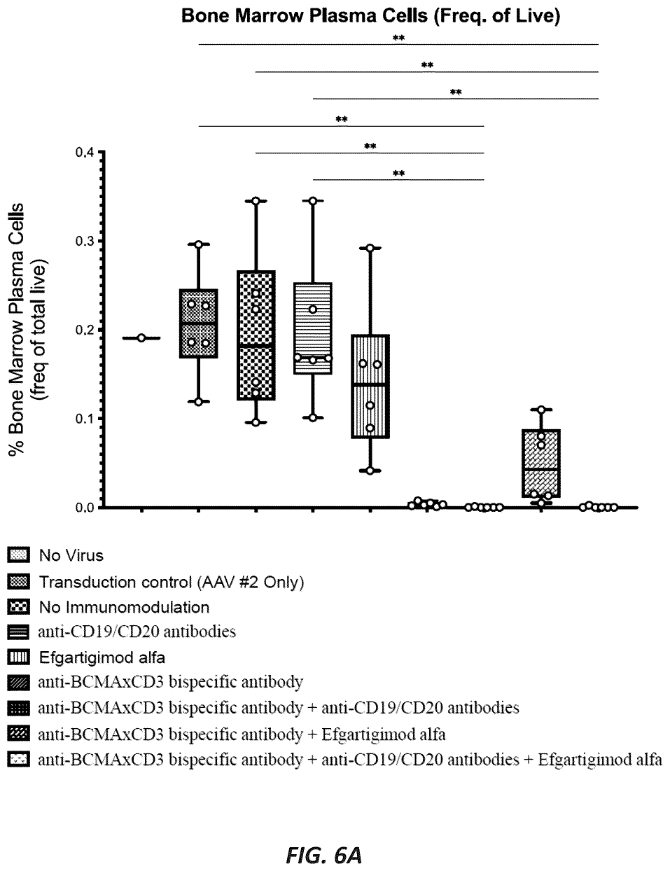

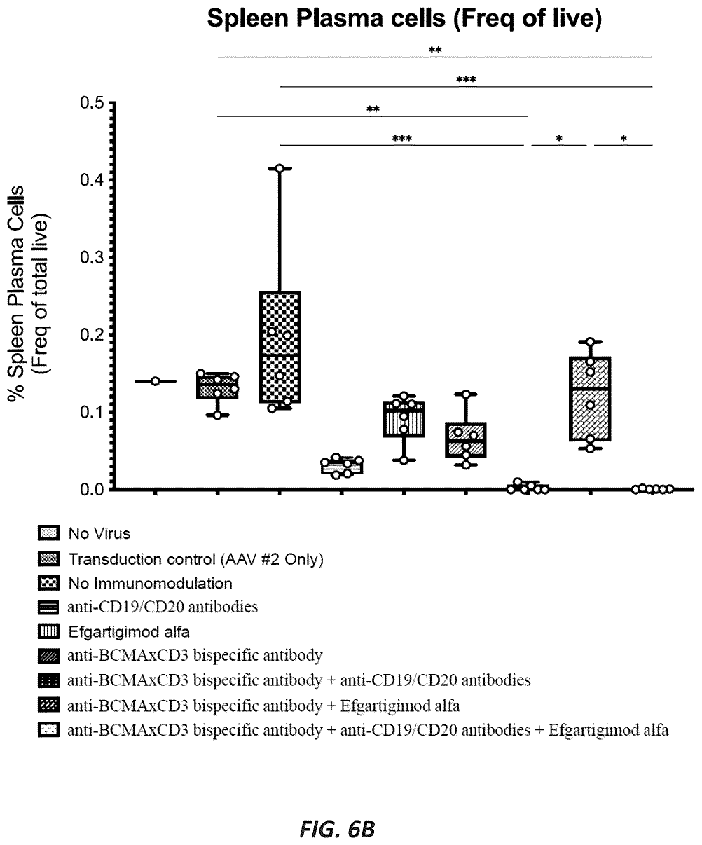

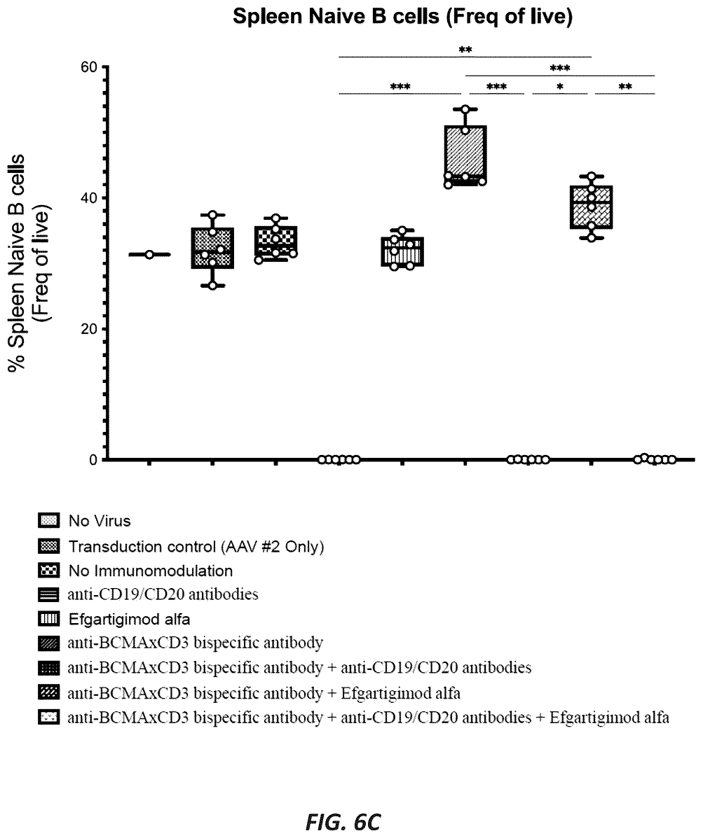

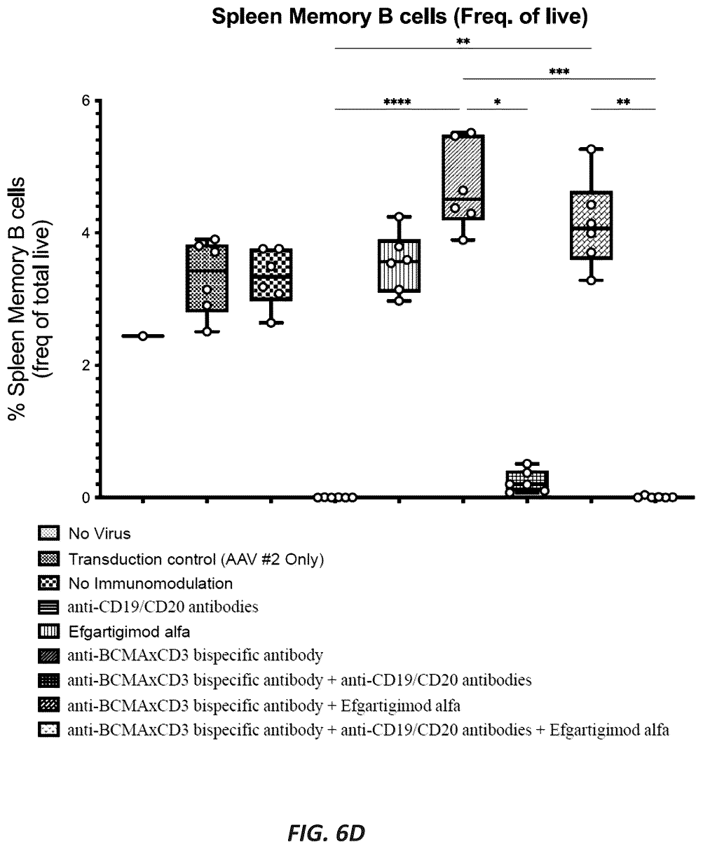

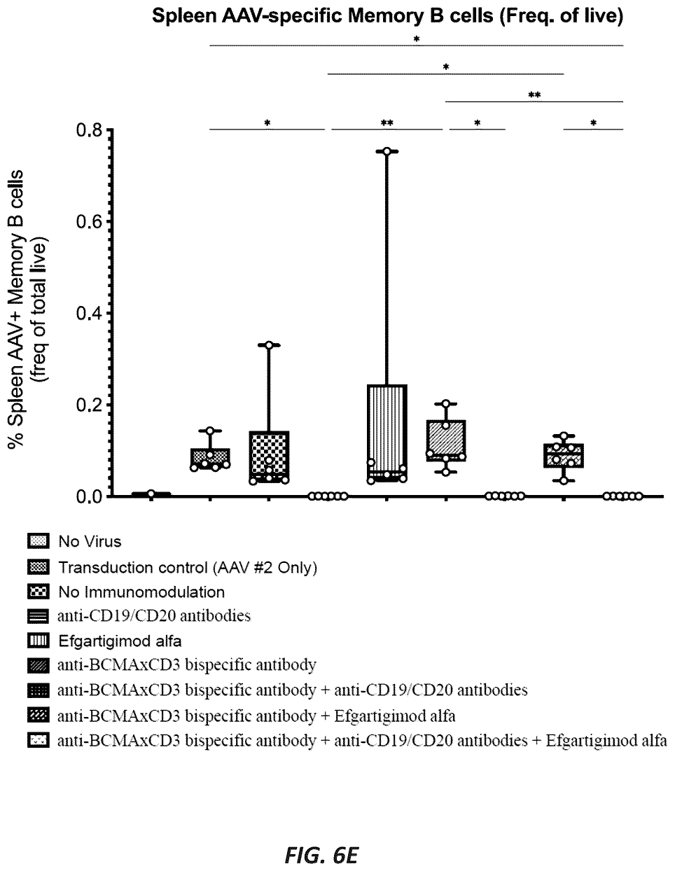

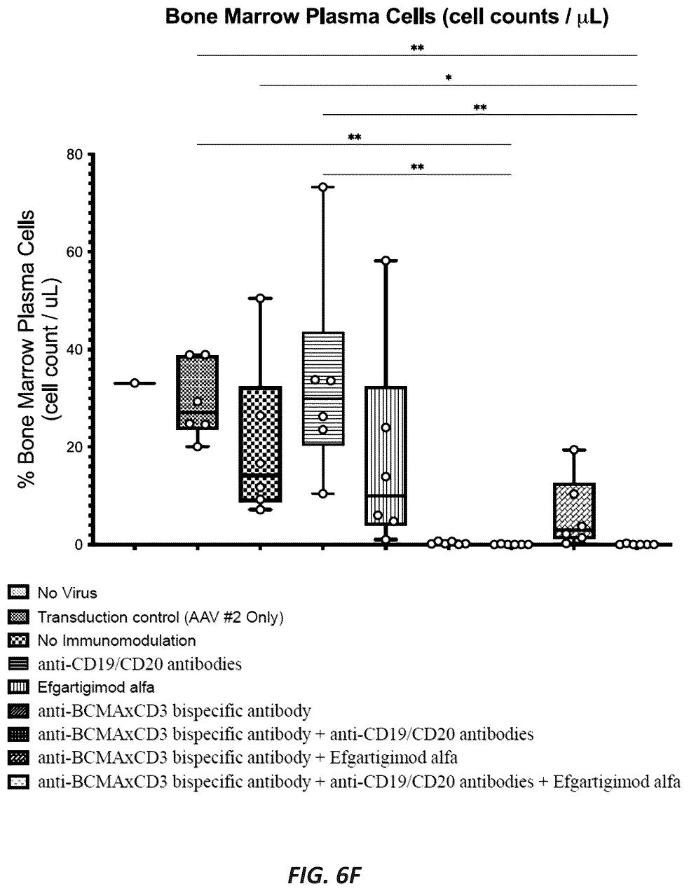

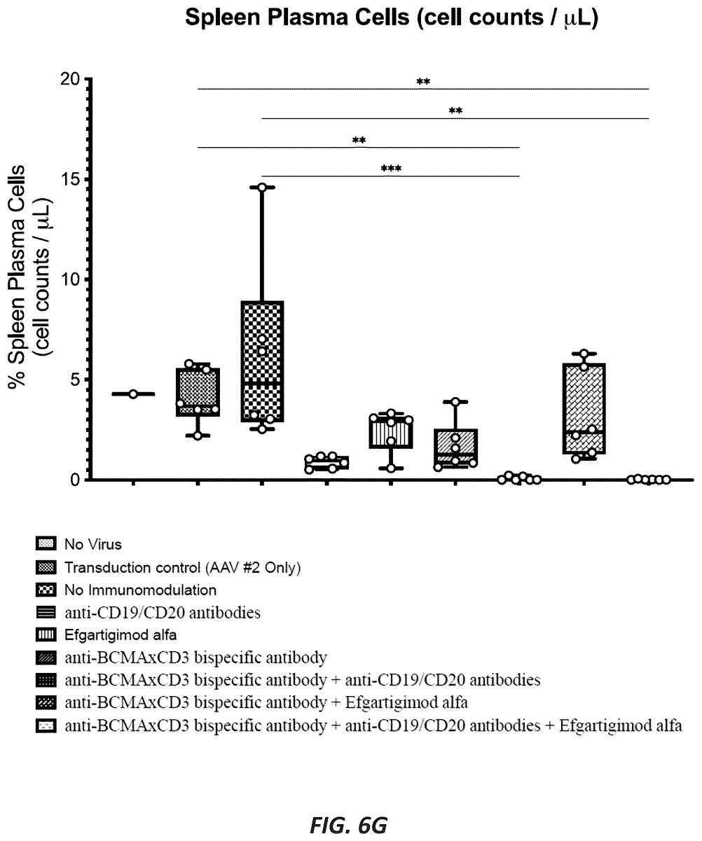

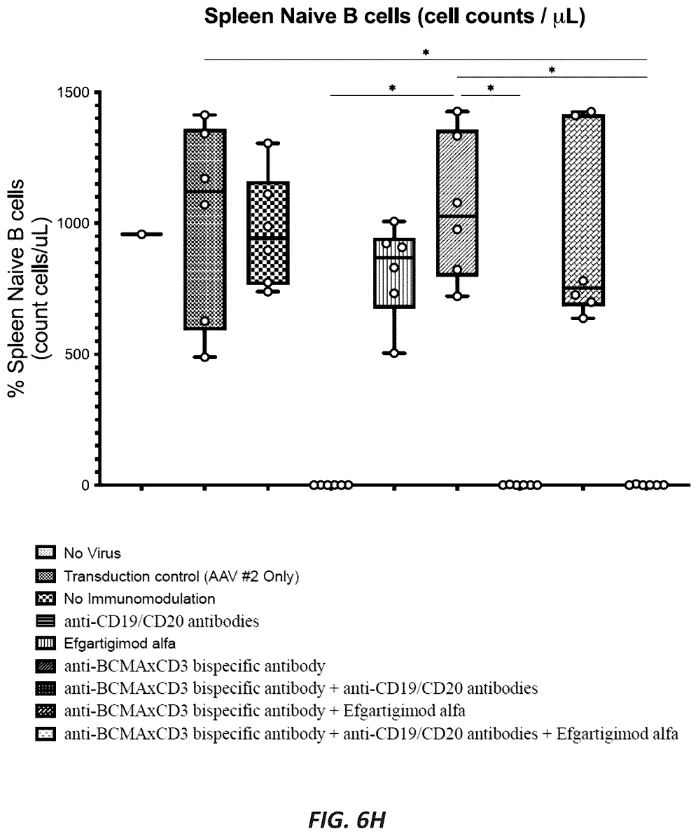

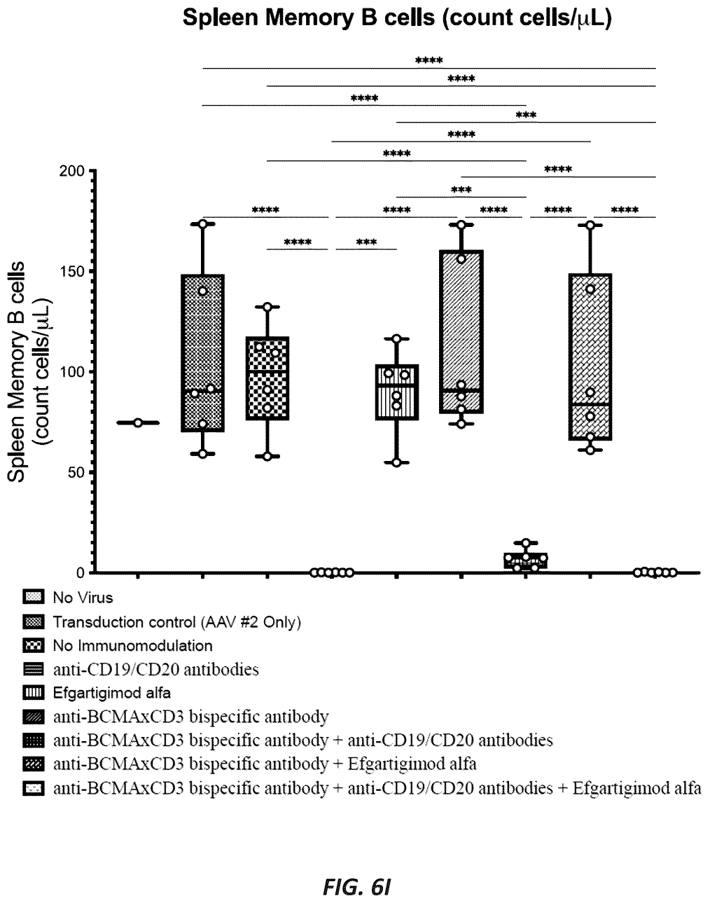

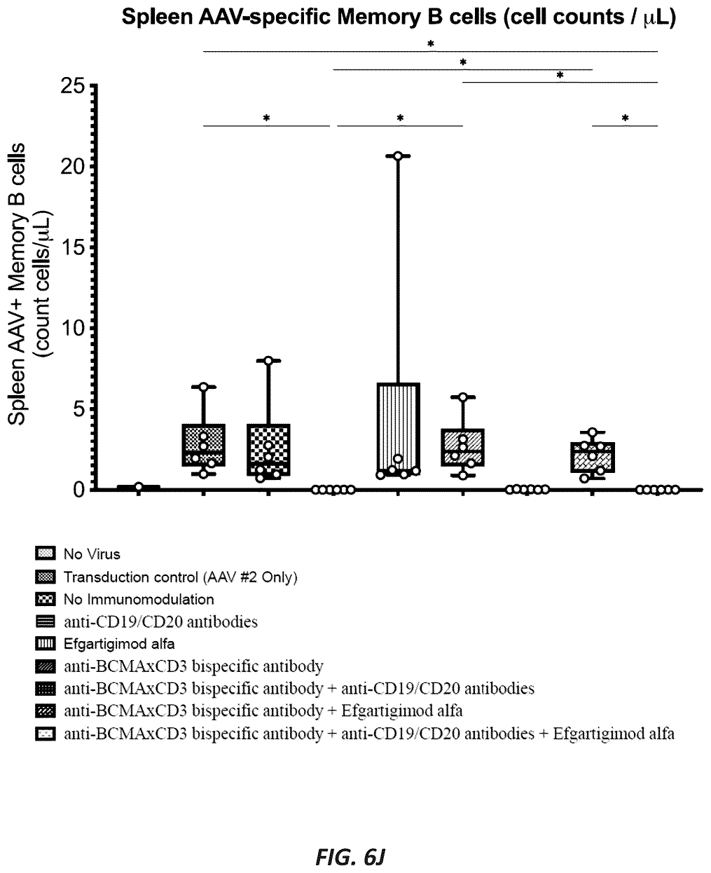

TABLE 13
The flow cytometry antibody staining panel used in FIGS. 6A-6J.

| | | | | | | |
|---|---|---|---|---|---|---|
| Reagent/Antigen | Conjugate | Reactivity | Host | Clone | Isotype | Supplier |

Pre-Stain

| | | | | | | |
|---|---|---|---|---|---|---|
| Live/Dead | Blue | N/A | N/A | N/A | N/A | Invitrogen |
| AAV8 | None | N/A | N/A | N/A | N/A | N/A |
| Fc Block | None | Human | Rat | 2.4G2 | N/A | TONGO |
| (CD16/32) | | | | | | biosciences |

Surface stain

| | | | | | | |
|---|---|---|---|---|---|---|
| CD38 | BUV395 | Mouse/ | Rat | 90/CD38 | IgG2a, k | BD |
| | | Human | | | | |
| CD138 | BV711 | Mouse | Rat | 281-2 | IgG2a, κ | BD |
| CD95 | BV421 | Mouse | Hamster | Jo2 | IgG2, λ | BD |
| GL-7 | PerCP- | Mouse/ | Rat | GL7 | IgM, k | Biolegend |
| | Cy5.5 | Human | | | | |
| IgD | BV786 | Mouse | Rat | 11-26c.2a | IgG2a, k | BD |
| IgA | FITC | Mouse | Rat | C10-3 | IgG1, κ | BD |
| IgG1 | BV510 | Mouse | Rat | A85-1 | IgG1, κ | BD |
| CD19 | BUV737 | Mouse | Rat | 1D3 | IgG2a, κ | BD |
| B220 | PE-Cy7 | Mouse | Rat | RA3-6B2 | IgG2a, κ | BD |
| CD98 | BV605 | Mouse | Hamster | H202-141 | IgG2a, κ | BD |
| CD1d | BUV563 | Mouse | Rat | WTH2 | IgG2a, κ | BD |
| Biotinylated | N/A | | Mouse | ADK8 | IgG2a | Progen |
| anti-AAV8 | | | | | | |
| IgG | | | | | | |
| TCRβ | APC | Mouse | Hamster | H57-597 | IgG2, λ1 | BD |
| CD200R3 | APC | Mouse | Rat | Ba13 | IgG2a, κ | Biolegend |
| Ly6G | APC | Mouse | Rat | 1A8-Ly6g | IgG2a, κ | eBioscience |
| CD49b | APC | Mouse | Rat | DX5 | IgM, κ | Biolegend |
| CD11b | APC | Mouse | Rat | M1/70 | IgG2b, κ | Biolegend |

Secondary Stain

| | | | | | | |
|---|---|---|---|---|---|---|
| Streptavidin | PE | N/A | N/A | N/A | N/A | Biolegend |

Intracellular stain

| | | | | | | |
|---|---|---|---|---|---|---|
| Light Chain k | BV650 | Mouse | Rat | 187.1 | IgG1, k | BD |
| Light Chain I | BV650 | Mouse | Rat | R26-46 | IgG2a, κ | BD |
| IgG1 | BV510 | Mouse | Rat | A85-1 | IgG1, k | BD |
| IgA | FITC | Mouse | Rat | C10-3 | IgG1, k | BD |

Analysis of B cell and plasma cell frequencies (FIGS. 6A-6E) and cell counts (FIGS. 6F-6J) in the bone marrow and spleen revealed that plasma cells were fully depleted in groups receiving anti-BCMA×CD3 bispecific antibody+anti-CD19/CD20 antibodies and the triple combination of anti-BCMA×CD3 bispecific antibody+efgartigimod alfa+anti-CD19/CD20 antibodies, consistent with reductions in anti-AAV8 titers seen in these groups. However, plasma cells were incompletely depleted in anti-BCMA×CD3 bispecific antibody groups that did not also receive anti-CD19/CD20 antibodies, suggesting either that ongoing plasma cell formation is a significant contributor to the anti-AAV8 IgG antibody pool in this model, or that mice developed anti-drug antibodies that react with anti-BCMA×CD3 bispecific antibody (a human IgG) that may have limited its therapeutic effect in the absence of B cell depletion (which would also deplete anti-human IgG-specific B cells). The effectiveness of anti-CD19/CD20 antibodies-mediated B cell depletion was also confirmed in analyses of naïve, memory, and AAV-specific B cells, with all subsets fully depleted except for a small subset of total memory B cells that were not depleted in the anti-BCMA×CD3 bispecific antibody+anti-CD19/CD20 antibodies combination group. Collectively, these data show that the titer reductions observed in the anti-BCMA×CD3 bispecific antibody+efgartigimod alfa+anti-CD19/CD20 antibodies are consistent with the expected mechanism of action, and that, by comparison, incomplete titer reductions observed in the anti-BCMA×CD3 bispecific antibody+efgartigimod alfa group may be explained by incomplete plasma cell depletion, possibly due to development of anti-drug antibodies reactive with anti-BCMA×CD3 bispecific antibody, efgartigimod, or both.

### Example 4. Use of BCMA×CD3 to Suppress Pre-Existing Anti-AAV nAbs Resulting from Wild-Type AAV Exposure and Enable Gene Insertion of an AAV Transgene Template

In this example, plasma cell depletion with BCMA×CD3 is utilized in combination with FcRn antagonism to suppress pre-existing antibody titers to AAV arising from natural AAV exposure, and consequently enable gene insertion of an AAV transgene template. Cynomolgus macaques with pre-existing total AAV antibody titers and AAV nAb titers ranging from low to high titer, are treated with BCMA×CD3 (REGN5458) with or without efgartigimod alfa (or alternative FcRn blocking therapeutic), and with or without CD20×CD3 bispecific antibody (REGN1979, or alternative B cell depletion therapeutic) weekly. Anti-AAV8 neutralizing and total antibody titers are evaluated biweekly. Monkeys treated with BCMA×CD3 alone are expected to show gradual decline in pre-existing anti-AAV8 antibody titers, but the effect is expected to be accelerated and magnified in groups additionally receiving FcRn antagonist, or groups additionally receiving FcRn antagonist and B cell depletion, with titers being reduced to sub-neutralizing levels. Monkeys not receiving BCMA×CD3 are not expected to show the same frequency, rate, or magnitude of titer decline. Several weeks later, monkeys are dosed intravenously with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific monkey genomic locus (e.g., intron 1 of monkey albumin). When transduction and expression of the therapeutic transgene are measured in liver by qPCR, DNA sequencing, and protein ELISA, results are expected to show that only monkeys receiving BCMA×CD3 exhibit productive gene insertion and measurable transgene mRNA and protein, whereas monkeys receiving no BCMA×CD3 fail to achieve AAV transduction, gene insertion, and expression. Monkeys receiving both BCMA×CD3 and FcRn blockade, or BCMA×CD3+FcRn blockade+B cell depletion, are expected to exhibit significantly higher levels of transduction, gene insertion, and expression than BCMA×CD3 treatment alone.

### Example 5. Use of BCMA×CD3 to Suppress Anti-AAV Neutralizing Antibody Responses and Enable Repeated Gene Insertion of an Identical AAV Template at a Single Genetic Locus

BCMA×CD3-mediated plasma cell depletion can allow for repeat administration of the same AAV template and LNP to achieve stepwise increase in transgene at a single genetic locus. In this example, BCMA-, CD3gamma-, CD3-delta, and CD3-epsilon humanized mice are intravenously treated with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific mouse genomic locus (e.g., intron 1 of mouse albumin). The insertion event is expected to result in measurable expression of the transgene. When anti-AAV8 antibody titers are evaluated several weeks after treatment by anti-AAV8 antibody ELISA, all AAV8 template treated mice are expected to exhibit strong antibody responses due to immune recognition of the AAV8 capsid. Several months later, mice are treated weekly with BCMA×CD3 (REGN5458), FcRn blocker (efgartigimod alfa or similar), and/or B cell depletion (anti-CD20+anti-CD19 antibody cocktail, or similar), individually or in combination. As controls, some mice are not treated with immunomodulation. When anti-AAV8 antibody titers are again measured by ELISA, results are expected to show that all mice treated with BCMA×CD3 show gradual reduction in anti-AAV8 antibody titer over the five-week treatment period, but that this reduction is more significant and rapid in mice that additionally receive FcRn blockade. Results are further expected to show that mice receiving triple combination of BCMA×CD3, FcRn blockade, and B cell depletion have even more accelerated and pronounced decreases in anti-AAV8 antibody titer, whereas mice not receiving BCMA×CD3 do not show the same frequency, rate, or magnitude of titer decline. Mice are then treated intravenously with the same AAV8 template vector and LNP that were initially administered. When transduction and expression of the therapeutic transgene are measured several weeks later in liver by qPCR, DNA sequencing, and protein ELISA, results are expected to show that mice receiving BCMA×CD3 exhibit an increase in levels of inserted template and augmented expression at the mRNA and protein levels, whereas mice receiving no BCMA×CD3 fail to achieve any additional increase in levels of template DNA insertion and expression. Mice receiving both BCMA×CD3 and FcRn blockade, or BCMA×CD3+FcRn blockade+B cell depletion are expected to exhibit a significantly greater increase in inserted template levels and expression over mice treated with BCMA×CD3 treatment alone. Additional dosings with the same AAV template and LNP are possible with repeat courses of BCMA×CD3 immunomodulation and AAV8+LNP administration.

### Example 6. Use of BCMA×CD3 to Suppress Anti-AAV Neutralizing Antibody Responses and Enable Repeated Gene Insertion of Multiple AAV Templates at Separate Genetic Loci

BCMA×CD3-mediated plasma cell depletion can allow for repeat administration of an AAV template and LNP to achieve multiple insertions at separate genomic locations. In this example, BCMA-, CD3gamma-, CD3-delta, and CD3-epsilon humanized mice are treated with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific mouse genomic locus (e.g., intron 1 of mouse albumin). The insertion event is expected to result in measurable expression of the transgene. When anti-AAV8 antibody titers are evaluated several weeks after treatment by anti-AAV8 antibody ELISA, all AAV8 template treated mice are expected to exhibit strong antibody responses due to immune recognition of the AAV8 capsid. Several months later, mice are treated weekly with BCMA×CD3 (REGN5458), FcRn blocker (efgartigimod alfa or similar), and/or B cell depletion (anti-CD20+anti-CD19 antibody cocktail, or similar), individually or in combination. As controls, some mice are not treated with immunomodulation. When anti-AAV8 antibody titers are again measured by ELISA, results are expected to show that all mice treated with BCMA×CD3 show gradual reduction in anti-AAV8 antibody titer over the five-week treatment period, but that this reduction is more significant and rapid in mice that additionally receive FcRn blockade. Results are further expected to show that mice receiving triple combination of BCMA×CD3, FcRn blockade, and B cell depletion have even more accelerated and pronounced decreases in anti-AAV8 antibody titer, whereas mice not receiving BCMA×CD3 do not show the same frequency, rate, or magnitude of titer decline. Mice are then treated with a second AAV8 template vector containing a unidirectional or bidirectional DNA template encoding a second distinct therapeutic transgene (e.g., a therapeutic human IgG1 targeting a viral antigen) and an LNP containing mRNA encoding SpCas9 and a gRNA specific for creating a specific double strand break at a specific mouse genomic locus, distinct from the first cut site. When transduction and expression of the second therapeutic transgene are measured in liver by qPCR, DNA sequencing, and protein ELISA, results are expected to show that only mice receiving BCMA×CD3 exhibit a second productive gene insertion event and measurable mRNA and protein of the second transgene, whereas mice receiving no BCMA×CD3 fail to achieve AAV transduction, gene insertion, and expression. Mice receiving both BCMA×CD3 and FcRn blockade, or BCMA×CD3+FcRn blockade+B cell depletion, are expected to exhibit significantly higher levels of transduction, gene insertion, and expression than BCMA×CD3 treatment alone. Additional gene insertions are possible at other genetic loci with repeat courses of BCMA×CD3 immunomodulation and AAV8+LNP administration.

### Example 7. Use of BCMA×CD3 in Combination with IgG-Degrading Enzyme to Suppress Pre-Existing Anti-AAV nAbs from Natural AAV Exposure and Enable Gene Insertion of an AAV Transgene Template

In this example, plasma cell depletion with BCMA×CD3 is utilized in combination with IgG degrading enzyme to suppress pre-existing antibody titers to AAV, such as can occur from natural AAV exposure, thereby enabling gene insertion of an AAV transgene template. Cynomolgus macaques with pre-existing total AAV antibody titers and AAV nAb titers ranging from low to high titer, are treated with BCMA×CD3 (REGN5458) with or without CD20×CD3 bispecific antibody (REGN1979 or alternative B cell depletion therapeutic) weekly for several weeks, followed by one or two doses of IgG-degrading enzyme (IdeS or similar). Anti-AAV8 neutralizing and total antibody titers are evaluated biweekly prior to IdeS administration, and daily following IdeS administration. Antibody titers are expected to decline gradually after BCMA×CD3 and BCMA×CD3+CD20×CD3 treatment, but show rapid and greater magnitude decline with additional IdeS treatment, with titers being reduced to sub-neutralizing levels. Monkeys not receiving BCMA×CD3 are not expected to show the same frequency, rate, or magnitude of titer decline. Monkeys are then dosed intravenously with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific monkey genomic locus (e.g., intron 1 of monkey albumin). When transduction and expression of the therapeutic transgene are measured in liver by qPCR, DNA sequencing, and protein ELISA, results are expected to show that only monkeys receiving BCMA×CD3 exhibit productive gene insertion and measurable transgene mRNA and protein, whereas monkeys receiving no BCMA×CD3 fail to achieve AAV transduction, gene insertion, and expression. Monkeys receiving both BCMA×CD3 and IdeS, or BCMA×CD3+IdeS+B cell depletion, are expected to exhibit significantly higher levels of transduction, gene insertion, and expression than BCMA×CD3 treatment alone.

### Example 8. Use of BCMA×CD3 in Combination with IgG-Degrading Enzyme to Suppress Anti-AAV Titers Following Recombinant AAV Exposure to Enable Gene Insertion of an AAV Transgene Template

In this example, plasma cell depletion with BCMA×CD3 is utilized in combination with IgG degrading enzyme to suppress high-titer pre-existing antibody titers to AAV, such as can occur from exposure to a recombinant AAV gene therapeutic, thereby enabling gene insertion of an AAV transgene template. AAV seronegative cynomolgus macaques are first treated with an AAV8 vector, which is expected to result in a high titer of neutralizing anti-AAV8 antibodies. Several months later, monkeys are treated with BCMA×CD3 (REGN5458) with or without CD20×CD3 bispecific antibody (REGN1979 or alternative B cell depletion therapeutic) weekly for several weeks, followed by one or two doses of IgG-degrading enzyme (IdeS or similar). Anti-AAV8 neutralizing and total antibody titers are evaluated biweekly prior to IdeS administration, and daily following IdeS administration. Antibody titers are expected to decline gradually after BCMA×CD3 and BCMA×CD3+CD20×CD3 treatment, but show rapid and greater magnitude decline with additional IdeS treatment, with titers being reduced to sub-neutralizing levels. Monkeys not receiving BCMA×CD3 are not expected to show the same frequency, rate, or magnitude of titer decline. Monkeys are then dosed intravenously with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific monkey genomic locus (e.g., intron 1 of monkey albumin). When transduction and expression of the therapeutic transgene are measured in liver by qPCR, DNA sequencing, and protein ELISA, results are expected to show that only monkeys receiving BCMA×CD3 exhibit productive gene insertion and measurable transgene mRNA and protein, whereas monkeys receiving no BCMA×CD3 fail to achieve AAV transduction, gene insertion, and expression. Monkeys receiving both BCMA×CD3 and IdeS, or BCMA×CD3+IdeS+B cell depletion, are expected to exhibit significantly higher levels of transduction, gene insertion, and expression than BCMA×CD3 treatment alone.

### Example 9. Use of BCMA×CD3 in Combination with Therapeutic Plasma Exchange to Suppress Pre-Existing Anti-AAV nAbs from Natural AAV Exposure and Enable Gene Insertion of an AAV Transgene Template

In this example, plasma cell depletion with BCMA×CD3 is utilized in combination with therapeutic plasma exchange (TPE) to suppress pre-existing antibody titers to AAV, such as can occur from natural AAV exposure, thereby enabling gene insertion of an AAV transgene template. Cynomolgus macaques with pre-existing total AAV antibody titers and AAV nAb titers ranging from low to high titer, are treated with BCMA×CD3 (REGN5458) with or without CD20×CD3 bispecific antibody (REGN1979 or alternative B cell depletion therapeutic) weekly for several weeks. Monkeys are then subjected to one or more rounds of TPE. Anti-AAV8 neutralizing and total antibody titers are evaluated prior to and following each round of TPE. Antibody titers are expected to decline gradually after BCMA×CD3 and BCMA×CD3+CD20×CD3 treatment, but show rapid and greater magnitude decline with additional TPE treatment, with titers expected to be reduced to sub-neutralizing levels. Monkeys not receiving BCMA×CD3 are not expected to show the same frequency, rate, or magnitude of titer decline. Monkeys are then dosed intravenously with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific monkey genomic locus (e.g., intron 1 of monkey albumin). When transduction and expression of the therapeutic transgene are measured in liver by qPCR, DNA sequencing, and protein ELISA, results are expected to show that only monkeys receiving BCMA×CD3 exhibit productive gene insertion and measurable transgene mRNA and protein, whereas monkeys receiving no BCMA×CD3 fail to achieve AAV transduction, gene insertion, and expression. Monkeys receiving BCMA×CD3 and TPE, or BCMA×CD3+B cell depletion+TPE, are expected to exhibit significantly higher levels of transduction, gene insertion, and expression than BCMA×CD3 treatment alone.

### Example 10. Use of BCMA×CD3 in Combination with Therapeutic Plasma Exchange to Suppress Anti-AAV Titers Following Recombinant AAV Exposure to Enable Gene Insertion of an AAV Transgene Template

In this example, plasma cell depletion with BCMA×CD3 is utilized in combination with therapeutic plasma exchange (TPE) to suppress high-titer pre-existing antibody titers to AAV, such as can occur from exposure to a recombinant AAV gene therapeutic, thereby enabling gene insertion of an AAV transgene template. AAV seronegative cynomolgus macaques are first treated with an AAV8 vector, which is expected to result in a high titer of neutralizing anti-AAV8 antibodies. Several months later, monkeys are then treated with BCMA×CD3 (REGN5458) with or without CD20×CD3 bispecific antibody (REGN1979 or alternative B cell depletion therapeutic) weekly for several weeks. Monkeys are then subjected to one or more rounds of TPE. Anti-AAV8 neutralizing and total antibody titers are evaluated prior to and following each round of TPE. Antibody titers are expected to decline gradually after BCMA×CD3 and BCMA×CD3+CD20×CD3 treatment, but show rapid and greater magnitude decline with additional TPE treatment, with titers being reduced to sub-neutralizing levels. Monkeys not receiving BCMA×CD3 are not expected to show the same frequency, rate, or magnitude of titer decline. Several weeks later, monkeys are dosed intravenously with an AAV8 template vector containing a promoterless unidirectional or bidirectional DNA template encoding a therapeutic transgene (e.g., human FIX) and an LNP containing mRNA encoding SpCas9 and a gRNA specific for creating a specific double strand break at a specific monkey genomic locus (e.g., intron 1 of monkey albumin). When transduction and expression of the therapeutic transgene are measured in liver by qPCR, DNA sequencing, and protein ELISA, results are expected to show that only monkeys receiving BCMA×CD3 exhibit productive gene insertion and measurable transgene mRNA and protein, whereas monkeys receiving no BCMA×CD3 fail to achieve AAV transduction, gene insertion, and expression. Monkeys receiving BCMA×CD3 and TPE, or BCMA×CD3+B cell depletion+TPE, are expected to exhibit significantly higher levels of transduction, gene insertion, and expression than BCMA×CD3 treatment alone.

### Example 11. Negative Impact of Efgartigimod on Anti-BCMA×CD3 Serum Drug Concentration, which is Partially Preventable with B Cell Depletion, is Consistent with Cross-Reactive Anti-Drug Antibody Formation

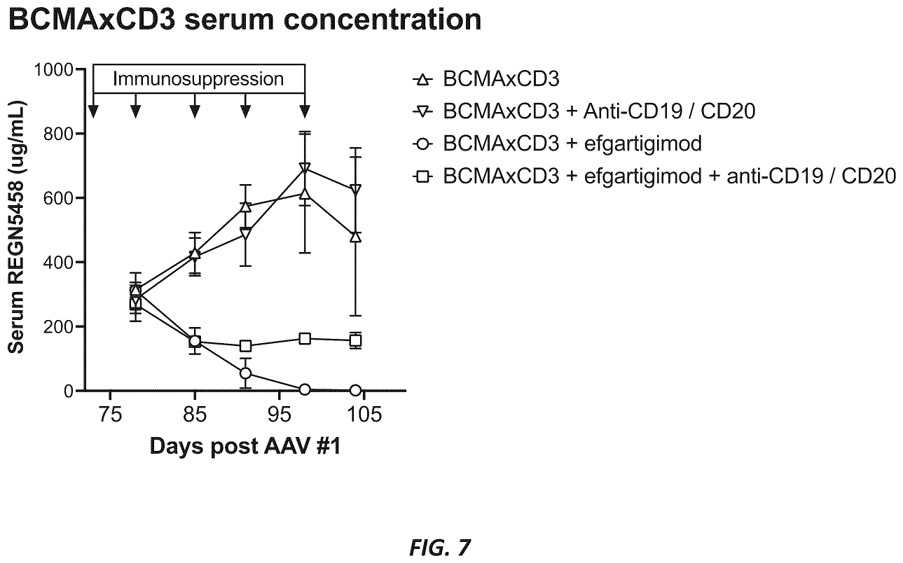

As described in Example 3, robust bone marrow plasma cell depletion was observed in anti-BCMA×CD3, anti-BCMA×CD3+anti-CD19/CD20, and anti-BCMA×CD3+anti-CD19/CD20+efgartigimod treatment groups that was consistent with the expected mechanism of action of anti-BCMA×CD3 observed in previous studies (Limnander et al. (2023) Sci. Transl. Med. 15(726):eadf9561, herein incorporated by reference in its entirety for all purposes). However, mice treated with anti-BCMA×CD3+efgartigimod, but not anti-CD19/CD20, unexpectedly showed incomplete bone marrow plasma cell depletion (FIG. 6A and FIG. 6F). To better understand the impact of efgartigimod on the efficacy of anti-BCMA×CD3, serum anti-BCMA×CD3 concentrations were evaluated during the immunomodulation treatment period. Repeated injections of anti-BCMA×CD3 in anti-BCMA×CD3 and anti-BCMA×CD3+anti-CD19/20 treatment groups resulted in expected increases in serum concentrations of BCMA×CD3 (FIG. 7). Moreover, mice treated with anti-BCMA×CD3+anti-CD19/20+efgartigimod showed significantly lower levels of serum anti-BCMA×CD3, as expected from the mechanism of action of efgartigimod, which blocks FcRn-mediated recycling of IgG, including recycling of therapeutic IgGs such as anti-BCMA×CD3. However, mice receiving anti-BCMA×CD3+efgartigimod without anti-CD19/20 exhibited an even greater rate of anti-BCMA×CD3 antibody clearance that resulted in complete loss of anti-BCMA×CD3 in serum starting 13 days after the initial efgartigimod dose (FIG. 7).

A faster clearance rate, reversible with B cell depletion, suggests that a humoral immune response may be contributing to drug clearance via development of anti-drug antibodies. Efgartigimod is a human IgG1 antibody fragment and is known to be immunogenic in mice. Anti-BCMA×CD3 is a human IgG4, which possesses >90% sequence identity to hIgG1. Therefore, it was concluded that efgartigimod, in the absence of additional B cell depletion, induced human IgG1/IgG4 cross-reactive anti-drug antibodies that accelerated anti-BCMA×CD3 clearance. It was further concluded that this anti-drug antibody response, in the absence of additional B cell depletion, negatively impacted BCMA-mediated bone marrow plasma cell depletion and consequently AAV titer reductions. Thus, the contribution of anti-CD19/20 antibodies to the efficacy of anti-BCMA×CD3 in non-human systems, in the presence of efgartigimod, may be model-specific through prevention of a xenogeneic anti-drug antibody response.

### Example 12. Plasma Cell Depletion in Combination with Neonatal Fc Receptor (FcRn) Blockade Potently Reduces Naturally-Occurring AAV Titers in AAV-Seropositive Cynomolgus Macaques

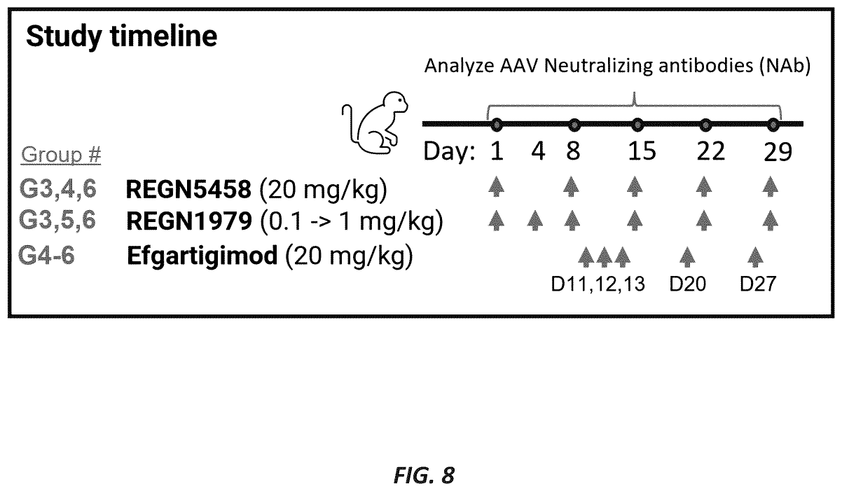

To evaluate whether plasma cell depletion with anti-BCMA×CD3 could similarly suppress naturally-occurring AAV titers arising from exposure to wild-type AAVs, as is common in humans, a non-human primate study was initiated with AAV8-seropositive macaques. Macaques were divided by AAV neutralizing antibody (nAb) titer into five treatment groups (n=3-5 each), each containing animals of high nAb titer (>1:450), as well as one group of seronegative control animals (n=3). Subsequently, animals were treated with various combinations of a plasma cell-depleting bispecific anti-BCMA×CD3 antibody (REGN5458, 20 mg/kg weekly), a B cell-depleting bispecific anti-CD20×CD3 antibody (REGN1979, 0.1 mg/kg on Study Day 1, 1 mg/kg on Study Days 4, 8, and weekly thereafter), and/or an FcRn blocker (efgartigimod, 20 mg/kg on Study Days 11, 12, 13, 20, and 27). Efgartigimod dosing was delayed relative to REGN5458 and REGN1979 dosing to minimize the impact of efgartigimod on REGN5458 and REGN1979 drug half-life due to FcRn blockade and/or cross-reactive anti-drug antibody development. NAb titers were analyzed weekly by cell-based neutralization assay, conducted by VRL Diagnostics (San Antonio, Texas). A schematic of the study design is shown in FIG. 8.

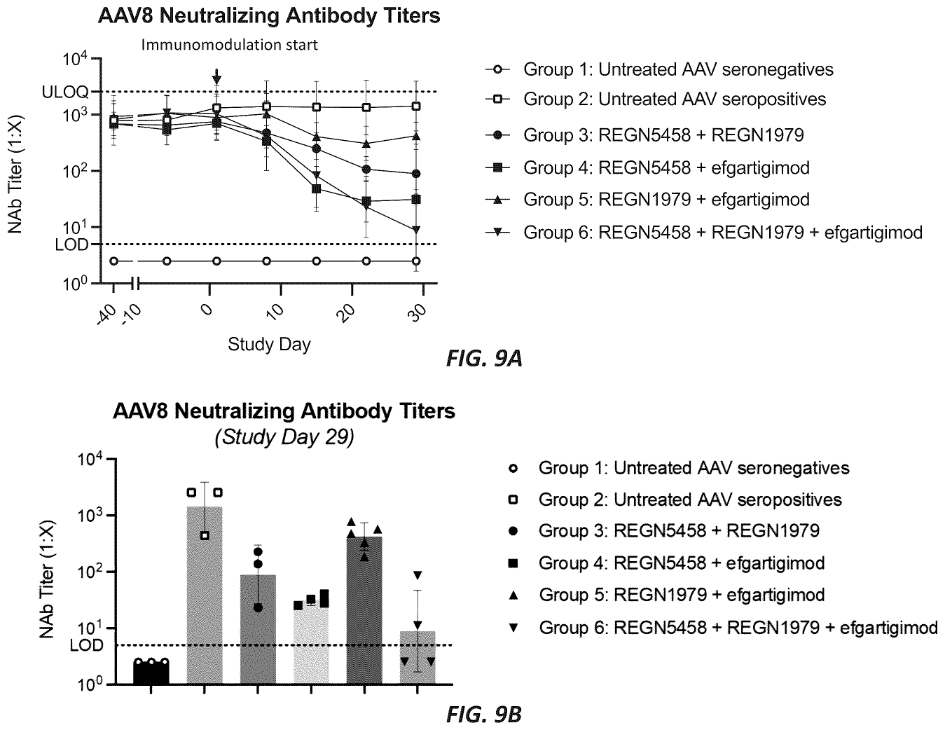

Longitudinal analysis of NAb titers revealed that only groups treated with immunomodulation cocktails containing REGN5458 showed substantive geometric mean titer reductions by ˜4 weeks after the start of immunomodulation. Whereas macaques receiving REGN5458-containing cocktails showed titer reductions of >10-fold, macaques receiving a cocktail containing efgartigimod and REGN1979, but not REGN5458, showed only marginal geometric mean nAb titer reduction of ˜2-fold (FIG. 9A). Similar to findings in mice, cocktails containing both a plasma cell depleter (REGN5458) and an FcRn blocker (efgartigimod), or all three immunomodulators, elicited the greatest titer reductions, with the triple combination of REGN5458, REGN1979, and efgartigimod inducing a >100-fold reduction in nAb titer (FIG. 9A). On Study Day 29, two animals in the triple combination group exhibited nAb titers below the limit of detection of the assay (FIG. 9B), suggesting that these animals could be successfully dosed with an AAV vector. Thus, plasma cell depletion is an effective strategy for suppressing naturally-occurring anti-AAV antibody titers, even of high-titer, to a level that is compatible with AAV dosing.

### Example 13. Prophylactic B Cell Depletion with Anti-CD20×CD3 More Effectively Suppresses the Antibody Response to AAV Vectors than Conventional Anti-CD20 Monoclonal Antibodies in Mice

B cell depletion with rituximab (or anti-CD20 mouse equivalents) has been evaluated in both preclinical and clinical settings as a strategy to prevent the antibody response to AAV and enable AAV vector re-administration. However, in both preclinical and clinical settings, prophylactic treatment with anti-CD20 antibodies has been shown to only partially suppress the antibody response to AAV vectors, and thus have failed to enable re-dosing (Salabarria et al. (2024) J. Clin. Invest. 134(1):e173510, Choi et al. (2023) J. Gene Med. 25(8):e3509, Meliani et al. (2018) Nat. Commun. 9(1):4098, and Unzu et al. (2012) J. Transl. Med., 10:122, each of which is herein incorporated by reference in its entirety for all purposes).

It was reasoned herein that rituximab and other anti-CD20 monoclonal antibodies may sub-optimally suppress anti-AAV antibody responses due to incomplete B cell depletion in secondary lymphoid tissues, the sites where antibody responses are initiated.

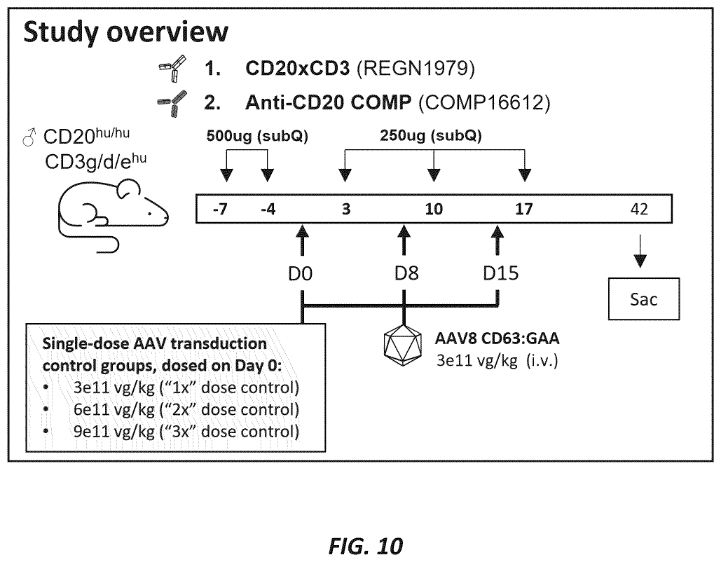

To test whether anti-CD20×CD3 bispecific antibodies could be more effective at preventing antibody response to AAV, the effectiveness of an anti-CD20×CD3 bispecific antibody (REGN1979) versus a rituximab comparator molecule (“Anti-CD20 COMP”) at suppressing the anti-AAV antibody response to repeated doses of AAV in mice was compared. A schematic of the study design is shown in FIG. 10. Specifically, mice humanized for CD20 and CD3 (gamma, delta, and epsilon chains) were treated prophylactically with two doses of either REGN1979 or anti-CD20 COMP (each 500 μg per mouse subQ (subcutaneously, s.c.)) on Study Days −7 and −4, then weekly for three weeks (250 μg per mouse per dose) starting on Study Day 3 to maintain B cell depletion. A separate group of mice received no B cell depleting agent (“No immunomodulation”). On Study Day 1, an AAV8 vector encoding acid alpha glucosidase fused to a single chain variable fragment targeting human CD63 (hereinafter, “anti-CD63 scFv:GAA”) was dosed intravenously at 3e 1 vector genomes (vg) per kilogram (kg), then subsequently re-administered at the same dose (3e 11 vg/kg) on Study Days 8 and 15. As AAV transduction controls, separate groups of mice were administered a single dose of AAV equal to one, two, or three doses (3e11, 6e11, or 9e11 vg/kg) on Study Day 1 only, in the absence of any B cell depletion agent.

Antibody titers were evaluated using anti-AAV8 IgM and IgG ELISA. Briefly, 96-well flat-bottom plates were coated with 1e9 vg/well recombinant AAV8 vector in DPBS overnight. The next day, plates were washed and blocked with a milk-based blocking agent (KPL SeraCare Milk Blocking Solution) for 1 hr. Serum samples were then diluted in the same blocker, beginning at an initial dilution of 1:300 followed by three-fold serial dilutions to a final dilution of 53,144,100. Diluted serum was then transferred to the assay plate and incubated overnight at 4° C. The next day, the assay plates were repeatedly washed prior to 1 hr incubation with a polyclonal secondary antibody targeting either mouse IgM (HRP-conjugated AffiniPure Goat Anti-Mouse IgM, μ chain specific, Jackson ImmunoResearch) or mouse IgG (HRP-conjugated anti-mouse Fcγ Fragment, Jackson ImmunoResearch), each diluted 1:5000 in DPBS+0.5% BSA. Plates were again repeatedly washed prior to development with TMB substrate solution. After 15-20 minutes, the reaction was stopped by addition of 2N phosphoric acid. Absorbance at 450 nm (OD450) was measured on a SpectraMax i3 plate reader (Molecular Devices, San Jose, CA). Anti-AAV8 IgM and IgG titers, defined as the dilution factor required to achieve an OD450 reading equal to 2-fold background, were determined and plotted using Prism software (v.10.1, GraphPad, Boston, MA).

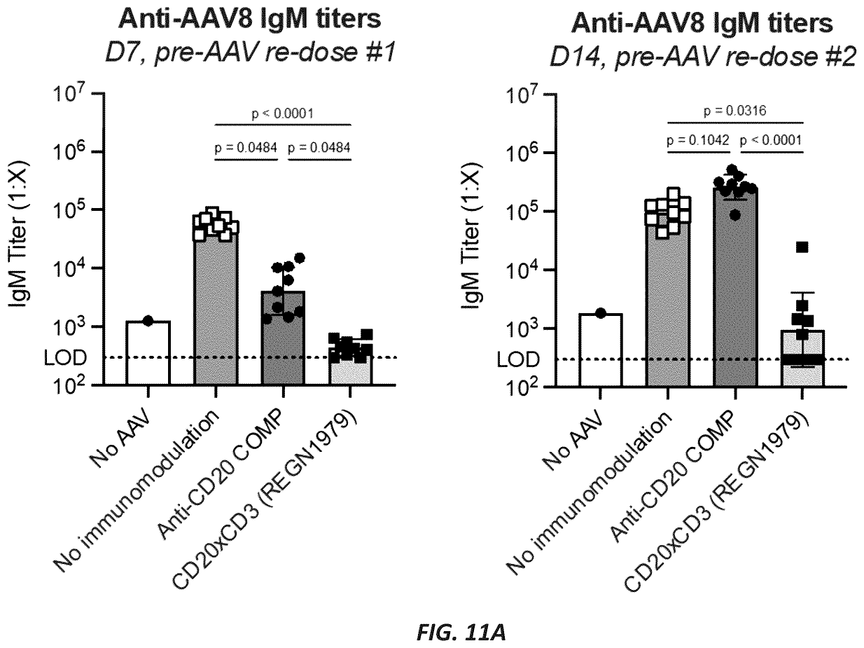

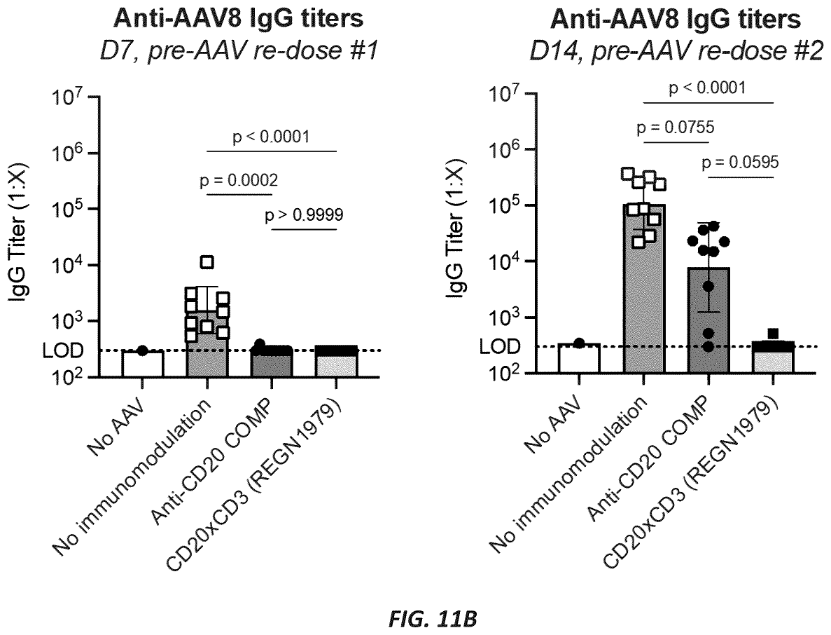

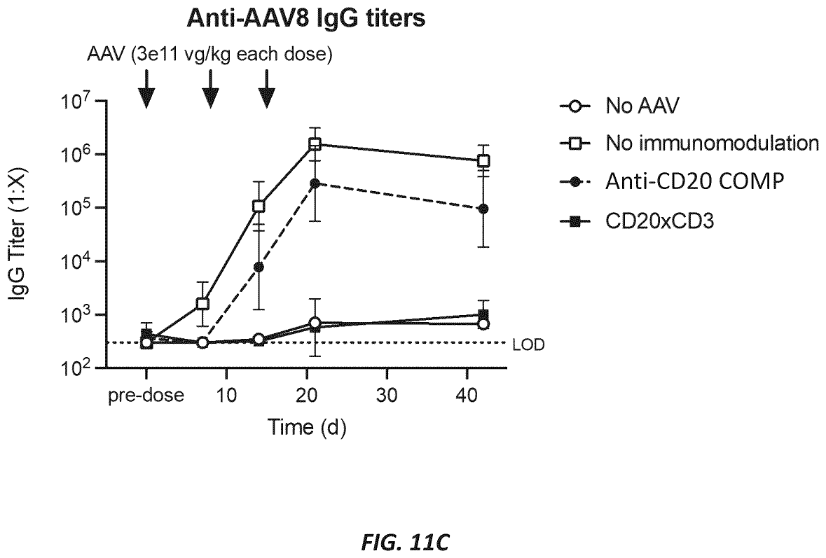

The results showed that AAV IgM titers just prior to AAV re-dose #1 (on Study Day 7) and AAV re-dose #2 (on Study Day 14) were more significantly suppressed in REGN1979-treated mice versus anti-CD20 COMP-treated mice, with REGN1979-treated mice exhibiting geometric mean titers that were essentially at undetectable levels at both timepoints (equivalent to or below that of a control AAV-naïve mouse) (FIG. 11A). By contrast, anti-CD20 COMP-treated mice showed only partial IgM titer suppression at the time of AAV re-dose #1, and no suppression at the time of AAV re-dose #2. Similarly, evaluation of anti-AAV8 IgG titers revealed that REGN1979-treated mice exhibited complete suppression of the anti-AAV8 IgG response at both timepoints, whereas anti-CD20 COMP-treated mice showed clearly detectable IgG titers by Study Day 14 (FIG. 11B). Longitudinal examination of anti-AAV IgG titers showed that REGN1979-treated mice maintained strong IgG titer suppression until study termination (Day 42), whereas anti-CD20 COMP-treated animals eventually mounted a strong IgG response that peaked only slightly below levels of control mice receiving no immunomodulation FIG. 11C. Thus, mice treated with anti-CD20×CD3 bispecific antibody show superior suppression of anti-AAV antibody titers versus mice receiving conventional anti-CD20 monoclonal antibody.

### Example 14. Prophylactic B Cell Depletion with Anti-CD20×CD3, but not Conventional Anti-CD20 Monoclonal Antibody, Enables Systemic AAV8 Vector Re-Administration in Mice

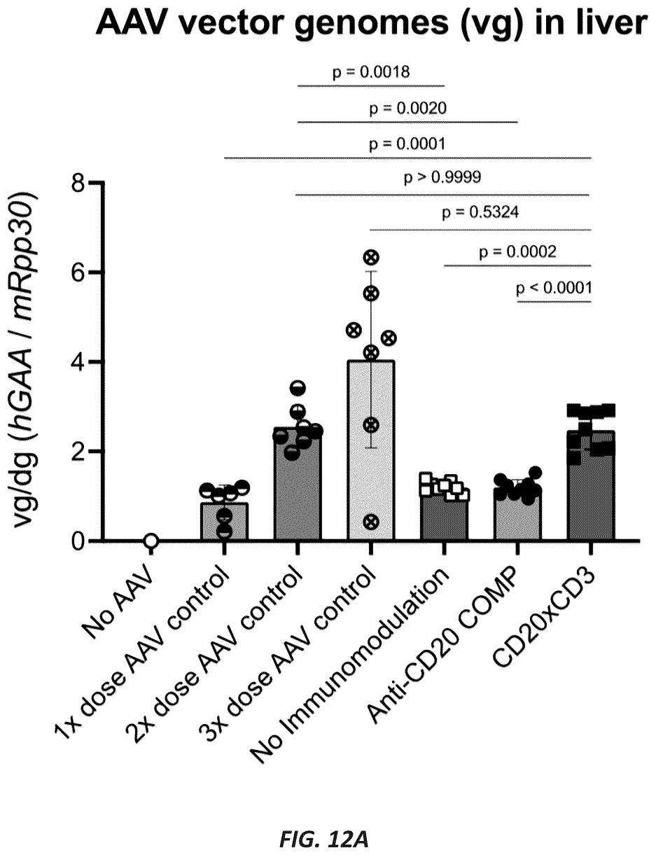

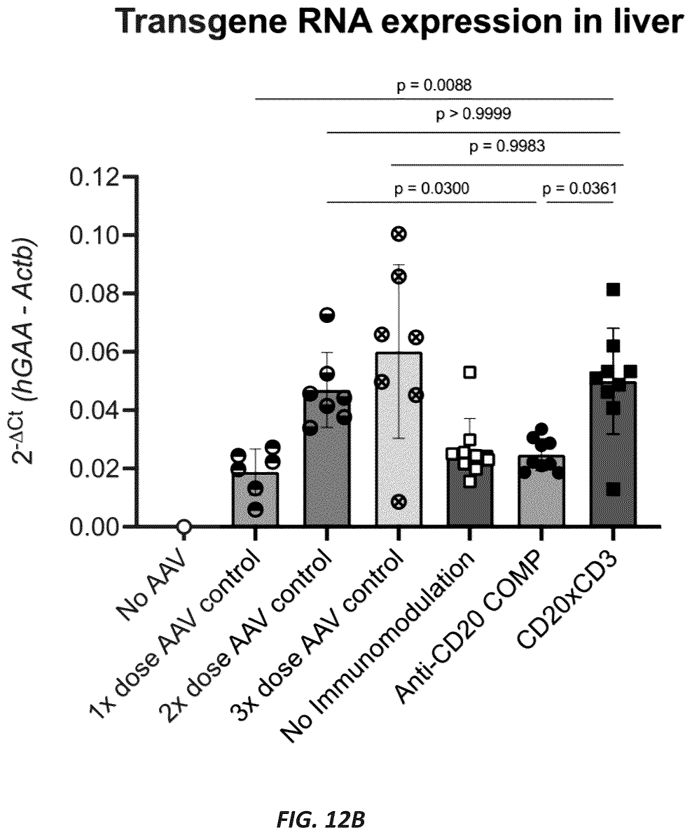

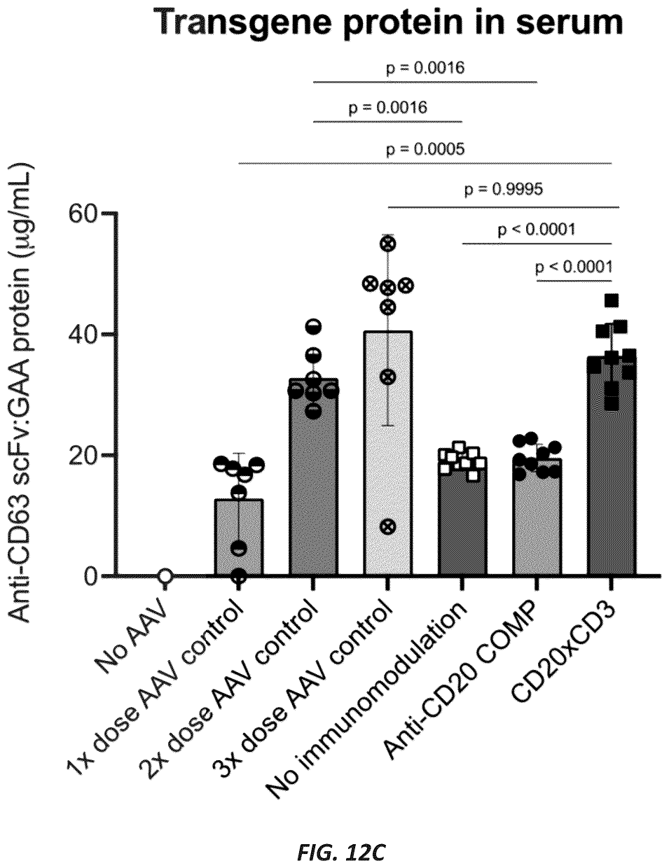

To evaluate whether the levels of anti-AAV titer suppression achieved by REGN1979 were sufficient to enable AAV re-dosing, mice from Example 13 were sacrificed on Study Day 42 for analysis of AAV transduction in liver. Analysis of AAV vector genomes by digital PCR revealed that REGN1979-treated mice exhibited levels of transduction in liver that were significantly higher than anti-CD20-treated mice and No Immunomodulation controls, and comparable to transduction control animals that received the equivalent of two AAV doses as a single dose (total 6e13 vg/kg) (FIG. 12A). By contrast, anti-CD20-treated mice showed no benefit versus no immunomodulation mice, failing to achieve meaningful levels of re-transduction. Transgene RNA expression analysis in liver (by RT-qPCR), and protein expression in serum (by ELISA) showed similar findings, with REGN1979-treated mice again exhibiting significantly higher levels of liver transgene RNA and protein expression versus anti-CD20-treated mice, at levels between that of transduction control mice receiving the equivalent of 2 or 3 AAV doses as a single dose (FIGS. 12B-12C). Taken together, these findings demonstrate that prophylactic B cell depletion with anti-CD20×CD3 bispecific antibody, but not conventional anti-CD20 monoclonal antibody, allows for successful AAV vector re-administration.

### Example 15. Prophylactic B Cell Depletion with Anti-CD20×CD3 Fully Suppresses the IgM, IgG, and Neutralizing Antibody (NAb) Response to AAV in Non-Human Primates

Previous reports have shown that B cell depletion with rituximab is insufficient to prevent antibody responses to systemic AAV in non-human primates, and ultimately fails to enable re-dosing (Unzu et al. (2012) J. Transl. Med., 10:122, herein incorporated by reference in its entirety for all purposes). It was thus investigated whether more potent B cell depletion with anti-CD20×CD3 bispecific antibody could achieve superior suppression of the antibody response post-AAV dosing, to levels that would facilitate re-dosing.

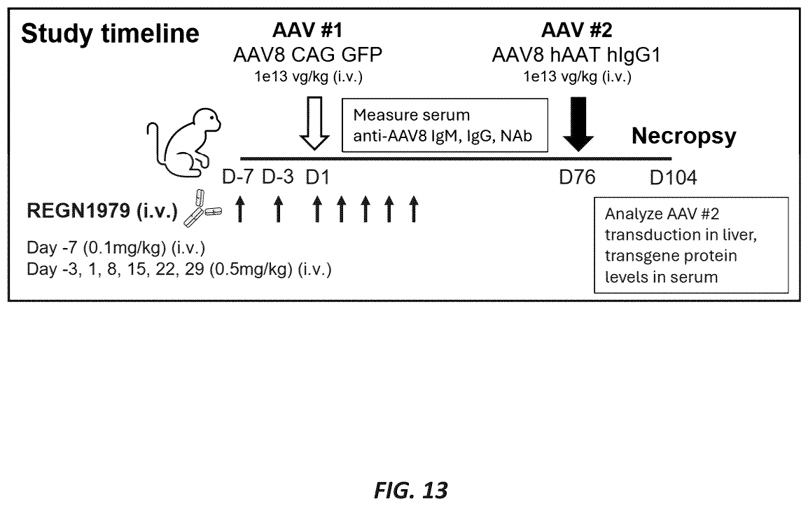

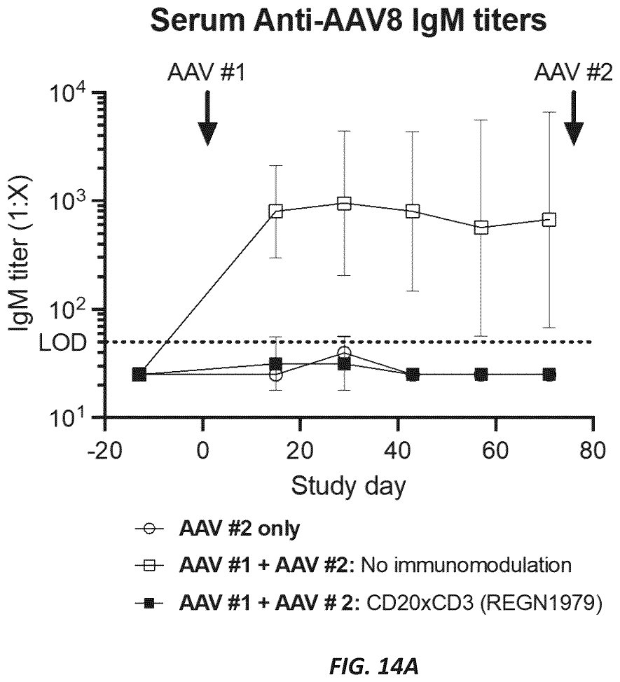

AAV8-seronegative cynomolgus macaques received either no immunomodulation (n=4) or anti-CD20×CD3 bispecific antibody (REGN1979, 0.1 mg/kg initial dose then 0.5 mg/kg weekly for 5 weeks; n=6). After three initial doses of REGN1979, animals in both groups were administered AAV8 CAG eGFP (“AAV #1”) intravenously (i.v.) at 1e13 vector genomes per kilogram (vg/kg). A third group was not administered the 1st AAV (“AAV #2 only”; n=6). Blood was sampled for 10 weeks for longitudinal antibody titer analysis. A schematic of the study design is shown in FIG. 13.

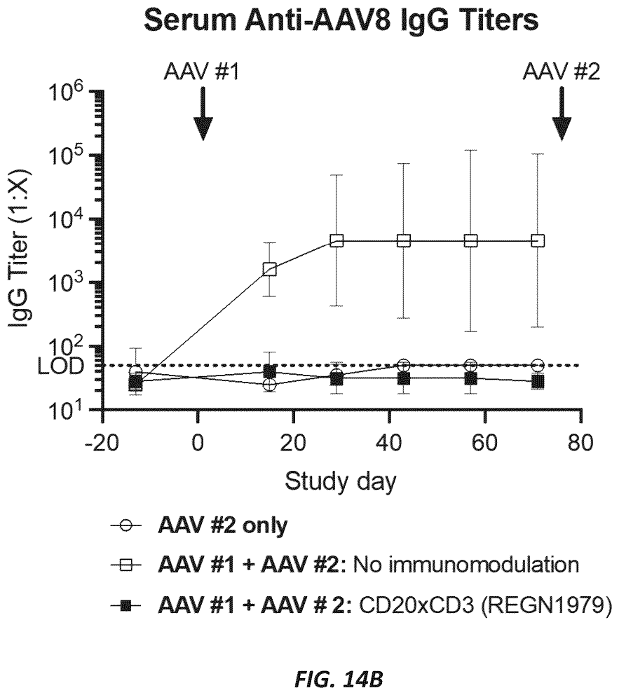

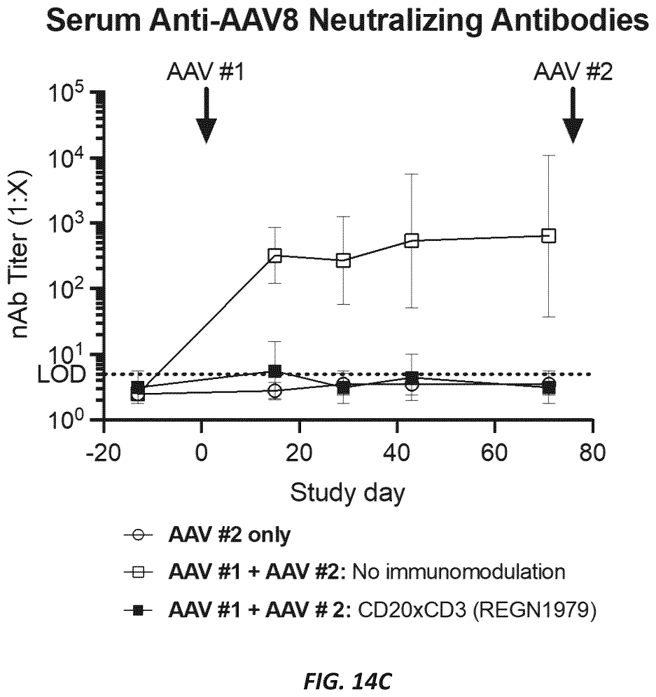

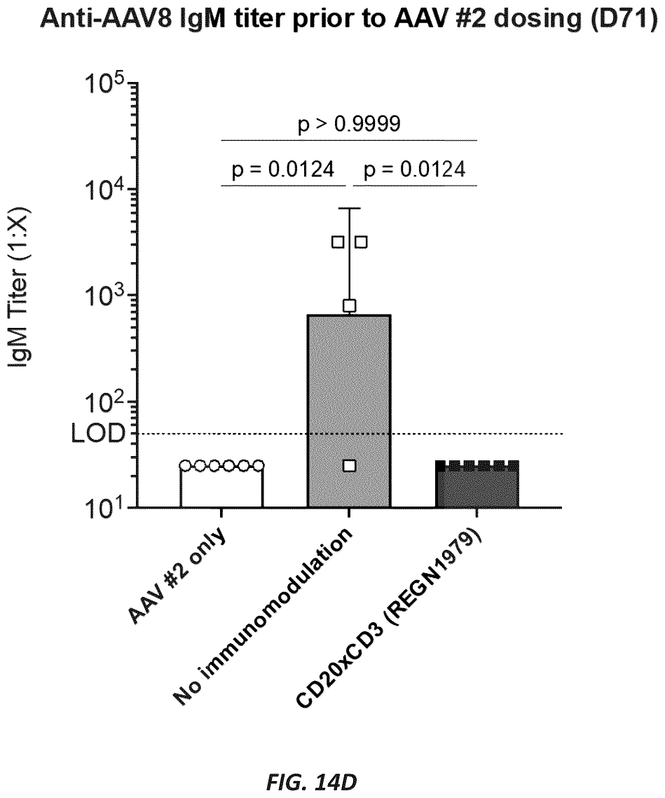

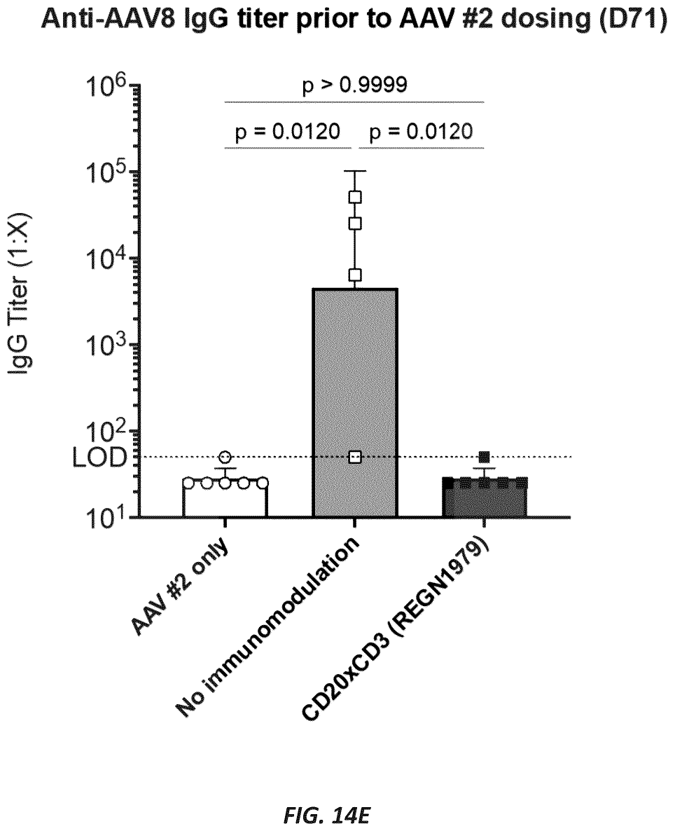

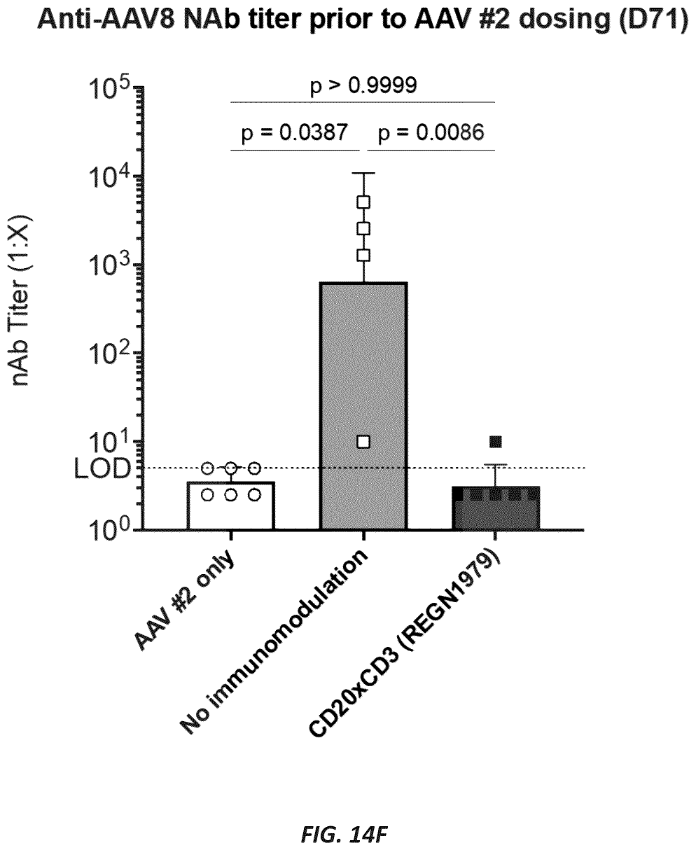

Analysis of anti-AAV IgM, IgG, and neutralizing antibody (nAb) titers was conducted by AAV titer ELISA or cell-based neutralization assay, according to standard methods known in the art. Results indicated that, as expected, macaques receiving no immunomodulation mounted a robust AAV8 IgM, IgG, and nAb response after the first AAV exposure (FIGS. 14A-14C). Strikingly, however, prophylactic B cell depletion with REGN1979 completely prevented an IgM, IgG, and nAb response for the entire 10-week analysis period (FIGS. 14A-14C). Individual animal IgM, IgG, and nAb titer data are shown for Study Day 71, 5 days prior to AAV re-dosing, in FIGS. 14D-14F. Thus, these data indicate that prophylactic B cell depletion with anti-CD20×CD3 bispecific antibody can fully prevent the antibody response to AAV in non-human primates, in contrast to comparable studies evaluating a conventional anti-CD20 therapeutic (rituximab), which failed to prevent the anti-AAV NAb response (Unzu et al. (2012) J. Transl. Med., 10:122, herein incorporated by reference in its entirety for all purposes).

### Example 16. Prophylactic B Cell Depletion with Anti-CD20×CD3 Enables Systemic AAV Re-Dosing in Non-Human Primates

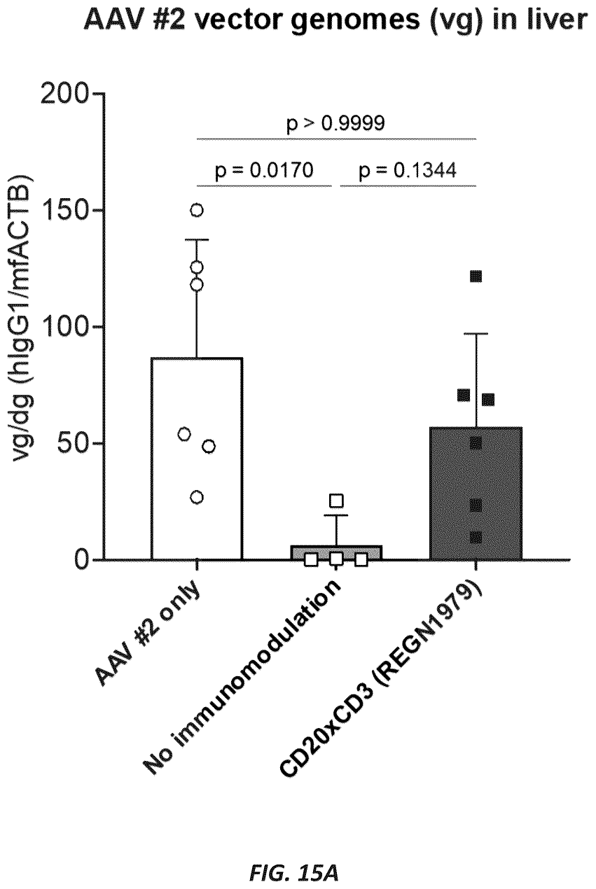

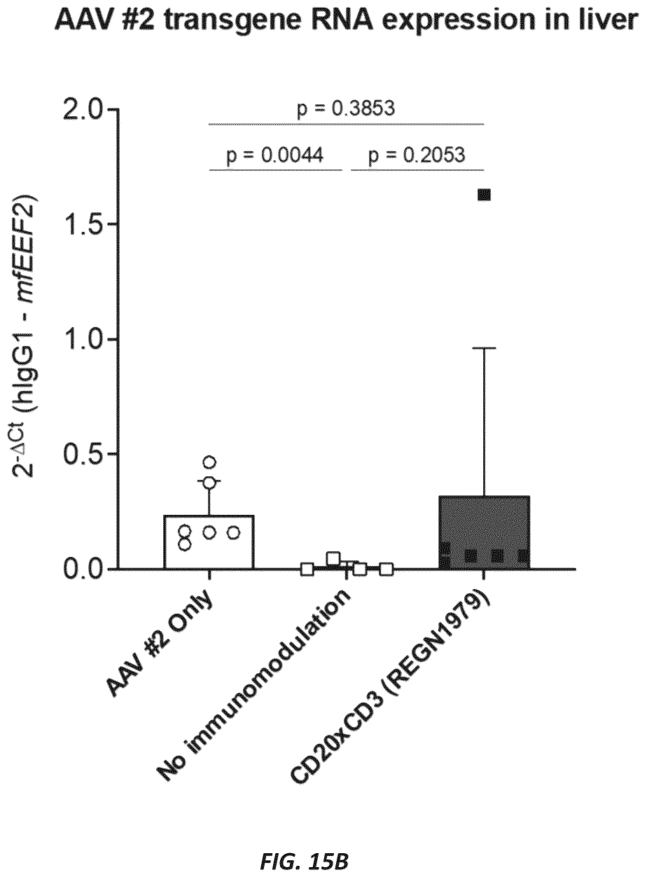

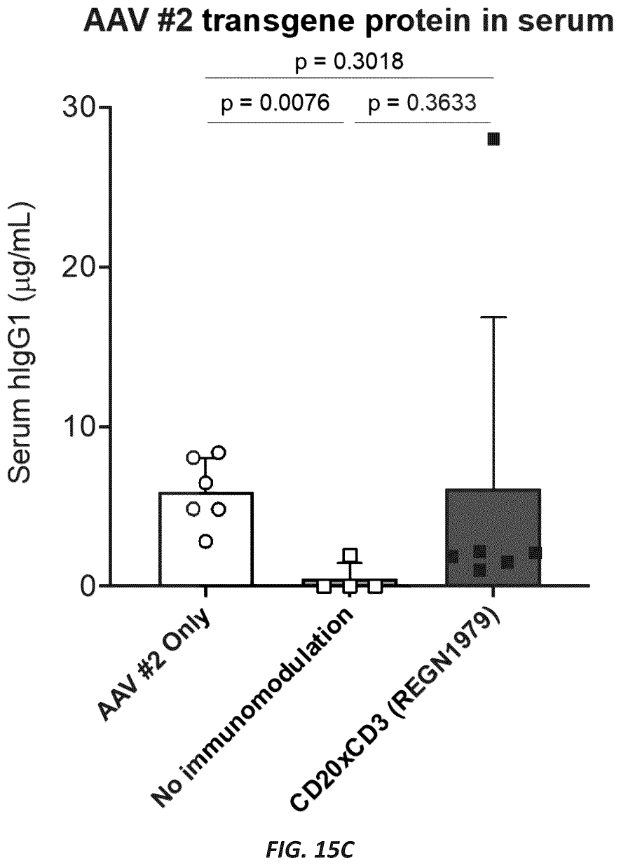

Next was evaluated whether the level of anti-AAV antibody titer suppression achieved in anti-CD20×CD3-treated macaques (described in Example 15) was sufficient to enable systemic AAV re-dosing. All animals (including “AAV #2 only” animals that had not received AAV #1) were intravenously (i.v.) administered a second AAV8 vector (“AAV #2”) encoding a secreted human IgG1 monoclonal antibody expressed from a liver-specific promoter at 1e13 vg/kg. Four weeks later, animals were necropsied, and liver transduction was evaluated by digital PCR. As expected, few detectable vector genomes per diploid genome were observed in control animals that previously received AAV #1 but no immunomodulation (FIG. 15A). By contrast, anti-CD20×CD3-treated macaques achieved robust transduction approaching that of previously-naïve control animals (“AAV #2 only”). Similarly, transgene mRNA (FIG. 15B) and hIgG1 protein (FIG. 15C) were readily detectable in liver and serum by RT-qPCR and ELISA, respectively, from CD20×CD3-treated and AAV #2 single-dosed control animals, but not re-dosed control animals that received no immunomodulation. Taken together, these findings show that systemic AAV vector re-administration is achievable via B cell depletion with bispecific anti-CD20×CD3 antibody, likely due to the deeper level of B depletion achievable in lymph nodes by this approach versus conventional anti-CD20 therapeutics.

### Example 17. Use of Anti-BCMA×CD3 in Combination with FcRn Antagonist to Suppress Pre-Existing Anti-AAV NAbs Resulting from Wild-Type AAV Exposure and Enable AAV-Mediated Gene Insertion

In this example, plasma cell depletion with anti-BCMA×CD3 is utilized in combination with FcRn antagonist to suppress pre-existing antibody titers to AAV arising from natural AAV exposure, and consequently enable gene insertion of an AAV transgene template. Cynomolgus macaques are divided by AAV neutralizing antibody (NAb) titer into several treatment groups, each containing animals that are AAV NAb seropositive (NAb titer>1:20), as well as one group of seronegative control animals (n=3). Subsequently, animals are treated with various combinations of a plasma cell-depleting bispecific anti-BCMA×CD3 antibody (REGN5458, 20 mg/kg weekly), a B cell-depleting bispecific anti-CD20×CD3 antibody (REGN1979, 0.1 mg/kg on Study Day 1, 1 mg/kg on Study Days 4, 8, and weekly thereafter), and/or an FcRn blocker (efgartigimod, 20 mg/kg on Study Days 11, 12, 13, 20, and 27). Efgartigimod dosing is delayed relative to REGN5458 and REGN1979 dosing to minimize the impact of efgartigimod on REGN5458 and REGN1979 drug half-life, due to FcRn blockade and/or cross-reactive monkey anti-human IgG antibody development. NAb titers are analyzed weekly by cell-based AAV neutralization assay, according to standard procedures in the field. Longitudinal analysis of AAV NAb titers reveals that all groups treated with REGN5458, or combinations thereof, show measurable NAb titer reductions. Animals treated with both REGN5458 and efgartigimod show more rapid and deep titer reductions than REGN5458 alone during the five week analysis period, due to accelerated turnover of IgGs resulting from FcRn blockade. Animals treated with all three agents (REGN5458, efgartigimod, and REGN1979) show the deepest reductions in titer, due to REGN1979-mediated B cell depletion, which both blocks development of cross-reactive anti-human IgG antibodies that limit the efficacy of efgartigimod and REGN5458 in monkeys, and eliminates AAV-specific antibody-secreting cells that are not depleted with REGN5458. Animals with lower starting NAb titers (approximately ≤1:100) drop to undetectable levels by Study Day 35 in all treatment groups containing REGN5458. Animals with moderate starting NAb titers (approximately >100 to ≤1:400) drop to undetectable levels by Study Day 35 in all treatment groups containing REGN5458 and efgartigimod. Animals with high starting NAb titers (approximately >1:400) drop to undetectable levels by Study Day 35 in the REGN5458+REGN1979+efgartigimod group.

On Study Day 36, control AAV8 seronegative animals, control AAV8 seropositive animals, and immunomodulation-treated animals are dosed intravenously at 1.5e13 vg/kg with a liver-tropic AAV8 vector containing a promoterless bidirectional DNA template encoding human FIX. The animals are also dosed intravenously with an LNP (1 mg/kg) encapsulating both an mRNA encoding SpCas9 and a gRNA specific for intron 1 of the Macaca fascicularis albumin gene. This strategy induces a double-strand break, insertion of the AAV template, and splicing of hFIX transgene mRNA to albumin exon 1, resulting in stable protein expression and secretion of hFIX. Subsequent analysis of liver transgene insertion by dPCR shows that REGN5458-treated animals that achieved undetectable NAb titers exhibit successful gene insertion at albumin intron 1, at roughly equivalent levels to positive control AAV seronegative animals. Analysis of transgene mRNA by RT-qPCR of liver biopsies show detectable hFIX transcript, at an equivalent level to previously AAV-naïve animals. Analysis of hFIX protein in plasma by ELISA reveals successful protein secretion into the blood at readily detectable levels, at equivalent levels to positive control AAV seronegative animals. Thus, anti-BCMA×CD3, and combination treatments thereof, can enable successful AAV-mediated gene insertion in the context of pre-existing NAbs from wild-type AAV exposure.

### Example 18. Use of Anti-BCMA×CD3 to Enable Repeated AAV-Mediated Gene Insertion of a Transgene into a Single Genetic Locus

Anti-BCMA×CD3-mediated plasma cell depletion can allow for repeat gene insertion of an AAV template into a single genetic locus. In this example, BCMA-, CD3gamma-, CD3-delta, and CD3-epsilon humanized mice are intravenously treated with an AAV8 template vector containing a promoterless bidirectional DNA template encoding human FIX transgene (hFIX) at 5e12 vg/kg on Study Day 1. Simultaneously, the mice are dosed intravenously with an LNP (0.5 mg/kg) encapsulating both an mRNA encoding SpCas9 and a gRNA specific for intron 1 of the mouse albumin gene, resulting in a double-strand break and insertion of the AAV hFIX template into mouse hepatocytes. Splicing of hFIX transgene mRNA to mouse albumin exon 1 results in stable protein expression and secretion of hFIX into the blood, detectable by ELISA in mouse plasma samples collected one month after AAV treatment. On Study Day 60, all AAV-treated mice are evaluated for anti-AAV8 IgG titers and are confirmed to possess high titers (>1:10,000). On Study Day 70, mice are divided into several treatment groups receiving either anti-BCMA×CD3 (REGN5458, 25 mg/kg weekly for five weeks), FcRn blocker (efgartigimod alfa, 20 mg/kg weekly for five weeks), and/or B cell depletion (anti-CD20+anti-CD19 antibody cocktail, 20 mg/kg each, weekly for five weeks), individually or in combination. In groups receiving efgartigimod in combination with REGN5458 and/or B cell depleting antibodies, the first efgartigimod dose is omitted. As controls, some mice are not treated with immunomodulation. Anti-AAV8 IgG titers are measured longitudinally by ELISA. Results show that by Study Day 105, all mice treated with REGN5458 show some reduction in anti-AAV8 IgG titer over the five-week treatment and analysis period. This reduction is more significant and rapid in mice that additionally receive efgartigimod, with some animals exhibiting undetectable anti-AAV8 IgG titers by Study Day 105. Mice receiving triple combination of REGN5458, efgartigimod, and anti-CD19/20 antibodies, show even more rapid and deep anti-AAV8 IgG titer reductions, with all mice exhibiting undetectable NAb titers by Study Day 105. Mice not receiving REGN5458 show only minor or negligible anti-AAV8 IgG titer decline. On Study Day 105, mice are treated intravenously with the same AAV8 template vector and LNP that was initially administered on Study Day 1, at the same dose, and in the same manner, to achieve increased levels of gene insertion at the same gene locus in mouse hepatocytes that had not previously been edited on the first dose. Mice are necropsied on Study Day 135 for analysis of transgene insertion and transgene expression. Analysis of liver transgene insertion by dPCR shows that REGN5458-treated animals that achieved undetectable anti-AAV8 IgG titers, including all mice in the REGN5458+efgartigimod+anti-CD19/20 antibody treatment group, exhibit gene insertion levels that are approximately double that of control mice that received only one dose of AAV8 hFIX template and LNP. Similarly, analysis of transgene mRNA in liver and protein in plasma by RT-qPCR and hFIX ELISA, respectively, show approximately double the level of expression as mice that received only a single dose of AAV8 hFIX template+LNP, indicating a successful second hFIX gene insertion treatment. By contrast, mice that received no immunomodulation, or immunomodulation that did not contain REGN5458, show the same level of gene insertion, hFIX transcript, and hFIX protein as control mice that received only a single dose of AAV8 hFIX template+LNP, indicating unsuccessful second hFIX gene insertion treatment. Thus, anti-BCMA×CD3, in combination with FcRn antagonism and B cell depletion, can successfully suppress pre-existing AAV8 IgGs resulting from previous AAV vector administration, thereby enabling repeat AAV-mediated gene insertion events at the same locus, with the same transgene.

### Example 19. Use of Anti-BCMA×CD3 to Enable Sequential AAV-Mediated Gene Insertion of Two Different Transgenes into Two Different Genetic Loci

Anti-BCMA×CD3-mediated plasma cell depletion can allow for repeat gene insertion of different AAV templates into separate genetic loci. In this example, BCMA-, CD3gamma-, CD3-delta, and CD3-epsilon humanized mice are intravenously treated with an AAV8 template vector containing a promoterless bidirectional DNA template encoding human FIX transgene (hFIX) at 1.5e13 vg/kg on Study Day 1. Simultaneously, animals are dosed intravenously with an LNP (1 mg/kg) encapsulating both an mRNA encoding SpCas9 and a gRNA specific for intron 1 of the mouse albumin gene, resulting in a double-strand break and insertion of the AAV hFIX template into mouse hepatocytes. Splicing of hFIX transgene mRNA to mouse albumin exon 1 results in stable protein expression and secretion of hFIX into the blood, detectable by ELISA in mouse plasma samples collected one month after AAV treatment. On Study Day 60, all AAV-treated mice are evaluated for anti-AAV8 IgG titers and are universally found to possess high titers (>1:10,000). On Study Day 70, mice are divided into several treatment groups receiving either anti-BCMA×CD3 (REGN5458, 25 mg/kg weekly for five weeks), FcRn blocker (efgartigimod alfa, 20 mg/kg weekly for five weeks), and/or B cell depletion (anti-CD20+anti-CD19 antibody cocktail, 20 mg/kg each, weekly for five weeks), individually or in combination. In groups receiving efgartigimod in combination with REGN5458 and/or B cell depleting antibodies, the first efgartigimod dose is omitted. As controls, some mice are not treated with immunomodulation. Anti-AAV8 IgG titers are measured longitudinally by ELISA. Results show that by Study Day 105, all mice treated with REGN5458 show some reduction in anti-AAV8 IgG titer over the five-week treatment and analysis period. This reduction is more significant and rapid in mice that additionally receive efgartigimod, with some animals exhibiting undetectable anti-AAV8 IgG titers by Study Day 105. Mice receiving triple combination of REGN5458, efgartigimod, and anti-CD19/20 antibodies, show even more rapid and deep anti-AAV8 IgG titer reductions, with all mice exhibiting undetectable NAb titers by Study Day 105. Mice not receiving REGN5458 show only minor or negligible IgG titer decline. On Study Day 105, mice are treated intravenously at 1.5e13 vg/kg with a second AAV8 vector containing a promoterless, unidirectional template encoding a human IgG monoclonal antibody. At the same time, mice are intravenously administered a second, distinct LNP encapsulating mRNA transcript for SpCas9 as well as a gRNA targeting a second safe harbor site within the genome. This creates another double strand break and facilitates a second gene insertion event in hepatocytes at a distinct locus. Expression of the second transgene occurs via splicing of the transgene coding sequence to the exon containing the protein secretion signal. As positive controls for gene insertion, separate groups of mice receive either only the first AAV vector+LNP or the second AAV vector+LNP. Mice are necropsied on Study Day 135 for analysis of transgene insertion and transgene expression. Analysis of liver transgene insertion by dPCR shows that REGN5458-treated mice that achieved undetectable anti-AAV8 IgG titers, including all mice in the REGN5458+efgartigimod+anti-CD19/20 antibody treatment group, exhibit levels of gene insertion at both sites equivalent to positive control mice (previously AAV-naïve mice that received only single dose of either AAV8 hFIX template+LNP or AAV8 hIgG1 template+LNP). Similarly, analysis of transgene mRNA in liver and protein in plasma by RT-qPCR and ELISA, respectively, reveals levels of both hFIX and hIgG1 transcript and protein that match positive control mice. By contrast, mice that received no immunomodulation, or immunomodulation that did not contain REGN5458, display no evidence of successful hIgG1 gene insertion or transgene expression. Thus, anti-BCMA×CD3, and combination treatments thereof, can successfully suppress pre-existing AAV8 IgGs resulting from previous AAV vector administration, thereby enabling repeat AAV-mediated gene insertion of different transgenes at different genetic loci.

### Example 20. Use of Anti-BCMA×CD3 in Combination with IgG-Degrading Enzyme to Suppress Pre-Existing Anti-AAV NAbs from Natural AAV Exposure and Enable AAV-Mediated Gene Insertion

Plasma cell depletion with anti-BCMA×CD3 can be utilized in combination with IgG-degrading enzyme (IdeS) to suppress pre-existing antibody titers to AAV arising from natural AAV exposure and consequently enable AAV-mediated gene insertion. In this example, two groups of AAV8-seropositive cynomolgus macaques of moderate to high NAb titer (≥1:300) are treated with two intravenous doses of REGN5458 at 20 mg per kg, one week apart (Study Days 1 and 8), to deplete plasma cells. Separately, two additional groups of seropositive animals with roughly equivalent AAV NAb titer distribution receive no REGN5458. On Study Day 15, one of the REGN5458-treated groups, and one of the untreated groups, receives an intravenous dose of IgG-degrading enzyme (IdeS) at 2 mg per kg. AAV NAb titers are evaluated three days later (Study Day 18) by cell-based AAV transduction assay, using standard practices in the field. These data show that animals receiving REGN5458 alone show reductions in AAV NAb titer, but still exhibit detectable NAbs. Similarly, animals that did not receive REGN5458 but subsequently received IdeS show reductions in NAb titers, but retain AAV NAbs at levels sufficient for AAV neutralization (≥1:20). By contrast, animals that received both REGN5458 and IdeS in sequence show undetectable NAb titers on Study Day 18. On Study Day 19, all animals (including seronegative control animals) are dosed intravenously at 1.5e13 vg/kg with a liver-tropic AAV8 vector containing a promoterless bidirectional DNA template encoding human FIX, as well as an LNP (1 mg/kg) encapsulating both an SpCas9 mRNA and a gRNA specific for intron 1 of the Macaca fascicularis albumin gene. This strategy is designed to induce a double-strand break and insertion of the AAV template, while subsequent RNA splicing of hFIX transgene coding sequence to albumin exon 1 results in stable protein expression and secretion of hFIX. Upon analysis of liver transgene insertion by dPCR, only animals receiving the combination of REGN5458+IdeS show levels of insertion approaching or equivalent to previously AAV-naïve (seronegative) animals. Similarly, when hFIX RNA levels are interrogated in liver biopsies by RT-qPCR, and hFIX protein levels by ELISA of plasma, only animals treated with REGN5458+IdeS show levels approaching or equivalent to previously AAV-naïve (seronegative) animals. Thus, sequential treatment with anti-BCMA×CD3 and IdeS can enable AAV-mediated gene insertion in the context of pre-existing NAbs resulting from wild-type AAV exposure.

### Example 21. Use of Anti-BCMA×CD3 in Combination with IgG-Degrading Enzyme to Suppress Anti-AAV Nab Titers Following Recombinant AAV Exposure to Enable AAV-Mediated Gene Insertion

Plasma cell depletion with anti-BCMA×CD3 can be utilized in combination with IgG-degrading enzyme (IdeS) to suppress NAb titers arising after exposure to a recombinant AAV therapeutic to enable successful AAV-mediated gene insertion. In this example, cynomolgus macaques are treated with an AAV8 GFP vector on Study Day 1. Subsequent NAb analysis on Study Day 60 reveals that all animals developed high anti-AAV8 NAb titers (>1:400). Subsequently, the animals are divided into four separate groups. Two of the groups are treated with two intravenous doses of REGN5458 at 20 mg per kg, one week apart (Study Days 67 and 74), to deplete plasma cells. Separately, two additional groups with roughly equivalent AAV NAb titer distribution receive no REGN5458. On Study Day 81, one of the REGN5458-treated groups, and one untreated group, receives an intravenous dose of IgG-degrading enzyme (IdeS) at 2 mg per kg. AAV NAb titers are evaluated three days later (Study Day 84) by cell-based AAV transduction inhibition assay, using standard practices in the field. These data show that animals receiving REGN5458 alone show reductions in AAV NAb titer, but still exhibit detectable NAbs. Similarly, animals that did not receive REGN5458 but subsequently received IdeS show reductions in NAb titers, but retain AAV NAbs at levels sufficient for AAV neutralization (≥1:20). By contrast, animals that received REGN5458 and then subsequently received IdeS on Study Day 81 show undetectable NAb titers on Study Day 84. On Study Day 85, all animals (including seronegative control animals) are dosed intravenously at 1.5e13 vg/kg with a liver-tropic AAV8 vector containing a promoterless bidirectional DNA template encoding human FIX, as well as an LNP (1 mg/kg) encapsulating both an SpCas9 mRNA and a gRNA specific for intron 1 of the Macaca fascicularis albumin gene. This strategy is designed to induce a double-strand break and insertion of the AAV template, while subsequent RNA splicing of hFIX transgene coding sequence to albumin exon 1 results in stable protein expression and secretion of hFIX. Upon analysis of liver transgene insertion by dPCR, only animals receiving the combination of REGN5458+IdeS show levels of insertion approaching or equivalent to previously AAV-naïve (seronegative) animals. Similarly, when hFIX RNA levels are interrogated in liver biopsies by RT-qPCR, and hFIX protein levels by ELISA of plasma, only animals treated with REGN5458+IdeS show levels approaching or equivalent to previously AAV-naïve (seronegative) animals. Thus, sequential treatment with anti-BCMA×CD3 and IdeS can enable AAV-mediated gene insertion in the context of high titer pre-existing NAbs resulting from a previous recombinant AAV exposure, such as AAV gene therapy or AAV-mediated gene editing.

### Example 22. Use of Anti-BCMA×CD3 in Combination with Therapeutic Plasma Exchange (TPE) to Suppress Pre-Existing Anti-AAV nAbs from Natural AAV Exposure and Enable AAV-Mediated Gene Insertion

Plasma cell depletion with anti-BCMA×CD3 can be utilized in combination with therapeutic plasma exchange (TPE) to suppress pre-existing antibody titers to AAV arising from natural AAV exposure and consequently enable AAV-mediated gene insertion. In this example, two groups of AAV8-seropositive cynomolgus macaques of moderate to high NAb titer (>1:300) are treated with two intravenous doses of REGN5458 at 20 mg per kg, one week apart (Study Days 1 and 8), to deplete plasma cells. Separately, two additional groups of seropositive animals with roughly equivalent AAV NAb titer distribution receive no REGN5458. On Study Day 15, one of the REGN5458-treated groups, and one of the untreated groups, undergoes multiple rounds of therapeutic plasma exchange (3 to 4 cycles in total), according to standard practice. Immediately following TPE, blood is drawn from all animals (including those that did not undergo TPE) for NAb evaluation by cell-based AAV transduction inhibition assay, according to standard practices in the field. These data show that animals receiving REGN5458 alone, but not TPE, show reductions in AAV8 NAb titer, and still exhibit detectable NAbs. Similarly, animals that did not receive REGN5458 but received TPE show reductions in AAV8 NAb titers, but retain AAV NAbs at levels sufficient for AAV neutralization (≥1:20). By contrast, animals that received REGN5458 and then TPE in sequence show undetectable AAV8 NAb titers on Study Day 18. Shortly following the completion of TPE, but after the blood draw, all animals (including seronegative control animals) are dosed intravenously at 1.5e13 vg/kg with a liver-tropic AAV8 vector containing a promoterless bidirectional DNA template encoding human FIX, as well as an LNP (1 mg/kg) encapsulating both an SpCas9 mRNA and a gRNA specific for intron 1 of the Macaca fascicularis albumin gene. This strategy is designed to induce a double-strand break and insertion of the AAV template, while subsequent RNA splicing of hFIX transgene coding sequence to albumin exon 1 results in stable protein expression and secretion of hFIX. Upon analysis of liver transgene insertion by dPCR, only animals receiving the combination of REGN5458+TPE show levels of insertion approaching or equivalent to previously AAV-naïve (seronegative) animals. Similarly, when hFIX RNA levels are interrogated in liver biopsies by RT-qPCR, and hFIX protein levels by ELISA of plasma, only animals treated with REGN5458+TPE show levels approaching or equivalent to previously AAV-naïve (seronegative) animals. Thus, sequential treatment with anti-BCMA×CD3 and TPE can enable AAV-mediated gene insertion in the context of pre-existing NAbs resulting from wild-type AAV exposure.

### Example 23. Use of Anti-BCMA×CD3 in Combination with Therapeutic Plasma Exchange (TPE) to Suppress Anti-AAV NAb Titers Following Recombinant AAV Exposure to Enable AAV-Mediated Gene Insertion

Plasma cell depletion with anti-BCMA×CD3 can be utilized in combination with therapeutic plasma exchange (TPE) to suppress AAV NAb titers arising after exposure to a recombinant AAV therapeutic and enable successful AAV-mediated gene insertion. In this example, cynomolgus macaques are treated with an AAV8 GFP vector on Study Day 1. Subsequent NAb analysis on Study Day 60 reveals that all animals developed high anti-AAV8 NAb titers (>1:400). Subsequently, the animals are divided into four separate groups. Two of the groups are treated with two intravenous doses of REGN5458 at 20 mg per kg, one week apart (Study Days 67 and 74), to deplete plasma cells. Separately, two additional groups of seropositive animals with roughly equivalent AAV NAb titer distribution receive no REGN5458. On Study Day 81, one of the REGN5458-treated groups, and one untreated group, undergoes multiple rounds of therapeutic plasma exchange (3 to 4 cycles in total), according to standard practice. Immediately following TPE, blood is drawn for NAb evaluation by cell-based AAV transduction inhibition assay, according to standard practices in the field. These data show that animals receiving REGN5458, but not TPE, show reductions in AAV NAb titer, and still exhibit detectable NAbs. Similarly, animals that did not receive REGN5458 but receive TPE show reductions in NAb titers, but retain AAV NAbs at levels sufficient for AAV neutralization (≥1:20). By contrast, animals that received REGN5458 and TPE in sequence show undetectable NAb titers on Study Day 18. Shortly following the completion of TPE, but after the blood draw, all animals (including seronegative control animals) are dosed intravenously at 1.5e13 vg/kg with a liver-tropic AAV8 vector containing a promoterless bidirectional DNA template encoding human FIX, as well as an LNP (1 mg/kg) encapsulating both an SpCas9 mRNA and a gRNA specific for intron 1 of the Macaca fascicularis albumin gene. This strategy is designed to induce a double-strand break and insertion of the AAV template, while subsequent RNA splicing of hFIX transgene to albumin exon 1 results in stable protein expression and secretion of hFIX. Upon analysis of liver transgene insertion by dPCR, only animals receiving the combination of REGN5458+TPE show levels of insertion approaching or equivalent to previously AAV-naïve (seronegative) animals. Similarly, when hFIX RNA levels are interrogated in liver biopsies by RT-qPCR, and hFIX protein levels by ELISA of plasma, only animals treated with REGN5458+TPE show levels approaching or equivalent to previously AAV-naïve (seronegative) animals. Thus, sequential treatment with anti-BCMA×CD3 and TPE can enable AAV-mediated gene insertion in the context high titer pre-existing NAbs resulting from a previous recombinant AAV exposure, such as AAV gene therapy or AAV-mediated gene editing.

### Example 24. Prophylactic Anti-CD20×CD3-Mediated B Cell Depletion for Enabling Repeated, Titratable Gene Insertion at the Same Locus with the Same AAV Vector

Prophylactic B cell depletion with anti-CD20×CD3 bispecific antibody can be used to prevent or mitigate an anti-AAV antibody response and allow for repeated, titratable dosing of a gene insertion therapeutic over the span of days, weeks, months, or years. This approach enables a single larger dose to be divided over two or more smaller doses to optimize for therapeutic levels of transgene expression, while also minimizing risk for dose-related toxicities.

In this example, mice are prophylactically dosed with anti-CD20×CD3 antibody (REGN1979) to systemically deplete B cells and enable repeated, titratable AAV-mediated gene insertion of the same transgene at the same genetic locus. Specifically, AAV-naïve/seronegative CD20- and CD3-humanized mice are dosed on Study Days −7 and −3 with two doses of REGN1979 subcutaneously at 500 μg per mouse to deplete B cells, then weekly thereafter at 250 μg per mouse to maintain B cell depletion. For comparison, additional groups of mice receive a B cell depleting anti-CD20 monoclonal antibody at equivalent doses to REGN1979, or no immunomodulation. On Study Day 0, an AAV8 vector encoding a promoterless, bidirectional DNA template encoding human Factor IX (hFIX) is administered intravenously at 5e12 vg/kg, one-third the optimal therapeutic dose. At the same time, a lipid nanoparticle (LNP) encapsulating both an mRNA transcript for SpCas9 as well as a gRNA targeting exon 1 of the mouse albumin gene is also dosed intravenously at 0.33 mg/kg, one-third the optimal therapeutic dose. This creates a double strand break and facilitates insertion of the hFIX cassette into the albumin locus in hepatocytes. Splicing of the hFIX coding sequence to albumin exon 1 leads to stable hFIX expression and secretion into the blood. Human FIX transgene protein expression is evaluated in mouse plasma by ELISA a week later. Subsequently, the same AAV and LNP are dosed a second time, in the same manner and dose, and hFIX levels are again measured in plasma a week later. Then, the same AAV and LNP are dosed a third time, in the same manner and dose, and hFIX levels are measured in plasma a week later. These plasma hFIX protein measurements reveal that each AAV and LNP administration resulted in discrete and approximately equivalent increases in hFIX levels, and the target therapeutic level of FIX is achieved after the third AAV+LNP cycle. Analysis of gene insertion by dPCR one week after the third AAV+LNP administration reveals that REGN1979 pre-treated mice receiving three separate AAV+LNP doses have approximately three times the AAV genomes and gene insertion levels as control mice receiving a single AAV+LNP dose, and approximately equivalent levels to mice receiving the full therapeutic AAV+LNP dose as a single dose. Analysis of transgene expression by qPCR matches transduction and gene insertion findings. Control mice that received repeated AAV and LNP dosing but did not receive REGN1979 pre-treatment, or received conventional anti-CD20 B cell depleting monoclonal antibody, exhibit gene insertion, RNA expression levels, and hFIX plasma protein levels equivalent to control mice receiving one-third of the optimal therapeutic dose, thereby showing no evidence of successful gene insertion beyond the first AAV+LNP treatment. Longitudinal analysis of anti-AAV8 IgM and IgG antibody titers show that REGN1979 pre-treated mice receiving repeated doses of AAV+LNP show minimal increase in antibody titers relative to baseline. By contrast, mice receiving AAV with anti-CD20 antibody, or receiving no immunomodulation, show robust anti-AAV8 IgM and IgG antibody responses. Thus, these data show that by preventing the anti-AAV IgM and IgG antibody response, anti-CD20×CD3 bispecific antibody pre-treatment, but not conventional anti-CD20 monoclonal antibodies, can enable titratable increases in AAV gene insertion via repeated administration of the same AAV template and LNP, until the desired therapeutic level of transgene expression is achieved.

### Example 25. Prophylactic Anti-CD20×CD3-Mediated B Cell Depletion for Enabling Repeated Gene Insertion of the Same Transgene at Different Loci

In this example, mice are prophylactically dosed with anti-CD20×CD3 antibody (REGN1979) to enable repeated AAV-mediated gene insertion of the same transgene at the distinct genetic loci. Specifically, AAV-naïve/seronegative CD20- and CD3-humanized mice are dosed on Study Days −7 and −3 with two doses of REGN1979 subcutaneously at 500 μg per mouse to deplete B cells, then weekly thereafter at 250 μg per mouse to maintain B cell depletion. For comparison, additional groups of mice receive a B cell depleting anti-CD20 monoclonal antibody at equivalent doses to REGN1979, or no immunomodulation. On Study Day 0, an AAV8 vector encoding a promoterless, bidirectional DNA template encoding human Factor IX (hFIX) is administered intravenously at 7.5e12 vg/kg. At the same time, a lipid nanoparticle (LNP) encapsulating both mRNA transcript for SpCas9 as well as a gRNA targeting exon 1 of the mouse albumin gene is also dosed intravenously at 0.5 mg/kg. This creates a double strand break and facilitates insertion of the hFIX cassette into the albumin locus in hepatocytes. Splicing of the hFIX coding sequence to albumin exon 1 leads to stable hFIX expression and secretion into the blood. Human FIX transgene protein expression is evaluated in mouse plasma by ELISA two weeks later and hFIX is readily detectable at expected therapeutic levels. On Study Day 30, a second AAV8 vector also containing a promoterless DNA template encoding hFIX is administered intravenously at 7.5e12 vg/kg. At the same time, mice are intravenously administered at 0.5 mg/kg a second, distinct LNP encapsulating mRNA transcript for SpCas9 and a gRNA targeting a second safe harbor site within the genome. This creates another double strand break and facilitates a second gene insertion event in hepatocytes. Expression of the second hFIX transgene occurs via splicing of the hFIX coding sequence to an exon containing a protein secretion signal. As positive controls for gene insertion, separate groups of mice receive either the first AAV+LNP vector or the second AAV+LNP vector only. Digital PCR analysis of gene insertion at both loci reveals that anti-CD20×CD3 pre-treated mice show successful gene insertion of hFIX at both loci at levels equivalent to positive controls. Transgene RNA expression measured by qPCR show twice as much hFIX transcript in anti-CD20×CD3-treated, re-dosed animals vs. single-dosed AAV+LNP control animals. Longitudinal plasma hFIX protein measurements show discrete and approximately equivalent increases in hFIX levels after each AAV and LNP administration, and terminal hFIX levels that are approximately twice that of single-dosed AAV+LNP control animals. By contrast, mice treated with anti-CD20 antibody or no immunomodulation show minimal to no detectable hFIX insertion at the second locus, and exhibit hFIX transcript and protein levels equivalent to single-dosed AAV+LNP control animals, indicating unsuccessful re-dosing. Longitudinal analysis of anti-AAV8 IgM and IgG antibody titers show that anti-CD20×CD3 pre-treated mice mounted minimal increase in antibody titers relative to baseline. By contrast, mice receiving AAV with anti-CD20 antibody, or receiving no immunomodulation, show robust anti-AAV8 IgM and IgG antibody responses. Thus, these data show that by preventing the anti-AAV IgM and IgG antibody response, anti-CD20×CD3 bispecific antibody pre-treatment, but not pre-treatment with conventional anti-CD20 monoclonal antibodies, can enable repeat AAV gene insertion of the same transgene at two distinct loci.

### Example 26. Anti-CD20×CD3-Mediated B Cell Depletion for Enabling AAV-Mediated Gene Insertion of Distinct Transgenes into Different Genetic Loci

In this example, mice are prophylactically dosed with anti-CD20×CD3 antibody (REGN1979) to enable repeated AAV-mediated gene insertion of two different therapeutic transgenes at different genetic loci. Specifically, AAV-naïve/seronegative CD20- and CD3-humanized mice are dosed on Study Days −7 and −3 with two doses of REGN1979 subcutaneously at 500 μg per mouse, then weekly thereafter at 250 μg per mouse to maintain B cell depletion. On Study Day 0, an AAV8 vector containing a promoterless, bidirectional DNA template encoding hFIX transgene is administered intravenously at 1.5e13 vg/kg. Simultaneously, a lipid nanoparticle (LNP) encapsulating both an mRNA transcript for SpCas9 as well as a gRNA targeting exon 1 of the albumin gene is also dosed intravenously at 1 mg/kg. This creates a double strand break and facilitates gene insertion at the albumin locus in hepatocytes. Splicing of the hFIX coding sequence to albumin exon 1 leads to stable hFIX expression and secretion into the blood. One month later, mice are intravenously administered at 1.5e13 vg/kg a second AAV8 vector encoding a human IgG1 monoclonal antibody. At the same time, mice are intravenously administered at 1 mg/kg a second LNP encapsulating mRNA transcript for SpCas9 as well as a distinct gRNA targeting a second safe harbor site within the genome. This creates another double-strand break and facilitates a second gene insertion event in hepatocytes at a distinct locus. Expression of the second transgene occurs via splicing of the hIgG1 transgene coding sequence to an exon containing a protein secretion signal. As positive controls for gene insertion, separate groups of mice receive either only the first AAV+LNP vector or the second AAV+LNP vector. Digital PCR analysis of gene insertion at both loci reveal that anti-CD20×CD3 pre-treated mice, but not mice that were administered anti-CD20 antibody or received no immunomodulation, show successful gene insertion at both loci equivalent to positive controls. Analysis of transgene expression by qPCR and protein expression of hFIX and hIgG1 in plasma by ELISA matches transduction and gene insertion findings. Longitudinal analysis of anti-AAV8 IgM and IgG antibody titers show that anti-CD20×CD3 pre-treated mice show no increase in antibody titers relative to baseline. By contrast, mice receiving AAV with anti-CD20 antibody, or receiving no immunomodulation, show robust anti-AAV8 IgM and IgG antibody responses. These data show that by preventing the anti-AAV IgM and IgG antibody response, anti-CD20×CD3 pre-treatment, but not pre-treatment with conventional anti-CD20 monoclonal antibodies, can enable AAV gene insertion of two distinct transgenes at two distinct loci.

1. A method of inserting a nucleic acid encoding a polypeptide of interest into a target genomic locus in a cell or a population of cells in a subject, comprising administering to the subject:
(a) a nucleic acid construct comprising a coding sequence for the polypeptide of interest;
(b) a nuclease agent or one or more nucleic acids encoding the nuclease agent, wherein the nuclease agent targets a nuclease target site in the target genomic locus; and
(c) an effective amount of a plasma cell depleting agent,
wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, and the nucleic acid construct is inserted into the target genomic locus.

2. A method of expressing a polypeptide of interest from a target genomic locus in a cell or a population of cells in a subject, comprising administering to the subject:
(a) a nucleic acid construct comprising a coding sequence for the polypeptide of interest;
(b) a nuclease agent or one or more nucleic acids encoding the nuclease agent, wherein the nuclease agent targets a nuclease target site in the target genomic locus; and
(c) an effective amount of a plasma cell depleting agent,
wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, the nucleic acid construct is inserted into the target genomic locus to create a modified target genomic locus, and the polypeptide of interest is expressed from the modified target genomic locus.

3. A method of treating an enzyme deficiency in a subject in need thereof, comprising administering to the subject:
(a) a nucleic acid construct comprising a coding sequence for a polypeptide of interest, wherein the polypeptide of interest comprises an enzyme to treat the enzyme deficiency;
(b) a nuclease agent or one or more nucleic acids encoding the nuclease agent, wherein the nuclease agent targets a nuclease target site in a target genomic locus; and
(c) an effective amount of a plasma cell depleting agent,
wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency.

4. A method of preventing or reducing the onset of a sign or symptom of an enzyme deficiency in a subject in need thereof, comprising administering to the subject:
(a) a nucleic acid construct comprising a coding sequence for a polypeptide of interest, wherein the enzyme deficiency is characterized by a loss-of-function of the polypeptide of interest;
(b) a nuclease agent or one or more nucleic acids encoding the nuclease agent, wherein the nuclease agent targets a nuclease target site in a target genomic locus; and
(c) an effective amount of a plasma cell depleting agent,
wherein the subject has preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency.

5.-131. (canceled)

132. A composition or combination comprising an effective amount of a plasma cell depleting agent in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus.

133.-233. (canceled)

234. A kit comprising the composition or combination of claim 132.

235. (canceled)

236. A method of inserting a nucleic acid encoding a polypeptide of interest into a target genomic locus in a cell or a population of cells in a subject, comprising administering to the subject:
(a) a nucleic acid construct comprising a coding sequence for the polypeptide of interest;
(b) a nuclease agent or one or more nucleic acids encoding the nuclease agent, wherein the nuclease agent targets a nuclease target site in the target genomic locus; and
(c) an effective amount of an anti-CD20×CD3 bispecific antibody or functional fragment thereof,
wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, and the nucleic acid construct is inserted into the target genomic locus.

237. A method of expressing a polypeptide of interest from a target genomic locus in a cell or a population of cells in a subject, comprising administering to the subject:
(a) a nucleic acid construct comprising a coding sequence for the polypeptide of interest;
(b) a nuclease agent or one or more nucleic acids encoding the nuclease agent, wherein the nuclease agent targets a nuclease target site in the target genomic locus; and
(c) an effective amount of an anti-CD20×CD3 bispecific antibody or functional fragment thereof,
wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, the nucleic acid construct is inserted into the target genomic locus to create a modified target genomic locus, and the polypeptide of interest is expressed from the modified target genomic locus.

238. A method of treating an enzyme deficiency in a subject in need thereof, comprising administering to the subject:
(a) a nucleic acid construct comprising a coding sequence for a polypeptide of interest, wherein the polypeptide of interest comprises an enzyme to treat the enzyme deficiency;
(b) a nuclease agent or one or more nucleic acids encoding the nuclease agent, wherein the nuclease agent targets a nuclease target site in a target genomic locus; and
(c) an effective amount of an anti-CD20×CD3 bispecific antibody or functional fragment thereof,
wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, the nucleic acid construct is inserted into the target genomic locus to create a modified target genomic locus, and the polypeptide of interest is expressed from the modified target genomic locus, thereby treating the enzyme deficiency.

239. A method of preventing or reducing the onset of a sign or symptom of an enzyme deficiency in a subject in need thereof, comprising administering to the subject:
(a) a nucleic acid construct comprising a coding sequence for a polypeptide of interest, wherein the enzyme deficiency is characterized by a loss-of-function of the polypeptide of interest;
(b) a nuclease agent or one or more nucleic acids encoding the nuclease agent, wherein the nuclease agent targets a nuclease target site in a target genomic locus; and
(c) an effective amount of an anti-CD20×CD3 bispecific antibody or functional fragment thereof,
wherein the subject does not have preexisting immunity to the nucleic acid construct, the polypeptide of interest, the nuclease agent, the one or more nucleic acids encoding the nuclease agent, or a delivery vehicle for the nucleic acid construct, the nuclease agent, or the one or more nucleic acids encoding the nuclease agent, and
wherein the nuclease agent cleaves the nuclease target site, the nucleic acid construct is inserted into the target genomic locus to create a modified target genomic locus, and the polypeptide of interest is expressed from the modified target genomic locus, thereby preventing or reducing the onset of the sign or symptom of the enzyme deficiency.

240.-332. (canceled)

333. A composition or combination comprising an effective amount of an anti-CD20×CD3 bispecific antibody or functional fragment thereof in combination with: (a) a nucleic acid construct comprising a coding sequence for the polypeptide of interest; and (b) a nuclease agent or one or more nucleic acids encoding the nuclease agent, wherein the nuclease agent targets a nuclease target site in a target genomic locus.

334.-407. (canceled)

408. A kit comprising the composition or combination of claim 333.

409. (canceled)
